(19) United States
(12) Patent Application Publication
(10) Pub. No.: US 2026/0008768 A1
(43) Pub. Date: Jan. 8, 2026

(54) UREA DERIVATIVES FOR INHIBITING NLRP3 AND USES THEREOF

(71) Applicant: VENTUS THERAPEUTICS U.S., INC., Waltham, MA (US)

(72) Inventors: Stéphane DORICH, Pointe-Claire (CA); Ramsay E. BEVERIDGE, Pointe-Claire (CA); Stéphane CIBLAT, Montreal (CA); Alexandre CÔTÉ, Laval (CA); Patrick CYR, Saint-Constant (CA); Miguel ST-ONGE, Saint-Lazare (CA)

(21) Appl. No.: 19/327,113

(22) Filed: Sep. 12, 2025

Related U.S. Application Data
(63) Continuation of application No. PCT/US2024/020215, filed on Mar. 15, 2024.
(60) Provisional application No. 63/490,962, filed on Mar. 17, 2023.
(60) Provisional application No. 63/519,069, filed on Aug. 11, 2023.

(51) Int. Cl. C07D 401/04; A61K 31/439; A61K 31/454; A61K 31/4545; A61K 31/55; C07D 401/14; C07D 403/04; C07D 403/14; C07D 413/14; C07D 471/04; C07D 471/08

(52) U.S. Cl. CPC C07D 401/04; A61K 31/439; A61K 31/454; A61K 31/4545; A61K 31/55; C07D 401/14; C07D 403/04; C07D 403/14; C07D 413/14; C07D 471/04; C07D 471/08

(57) ABSTRACT

The present disclosure relates to compounds of Formula (I-B):

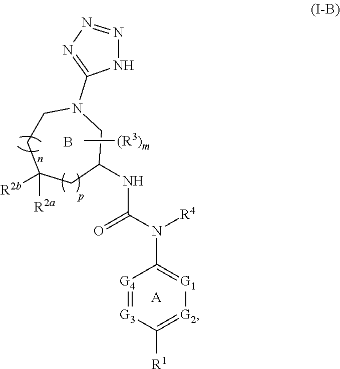

and pharmaceutically acceptable salts and tautomers thereof, wherein Ring A, Ring B, R¹, R^{2a}, R^{2b}, R³, R⁴, m, n, and p are described herein, and methods of preparation, methods of treatment and prevention, and pharmaceutical compositions comprising same. The present disclosure further relates to the use of the compounds of Formula (I-B), and pharmaceutically acceptable salts and tautomers thereof, in the treatment or prevention of NLRP3-related diseases and disorders.

### RELATED APPLICATIONS

This application is a continuation of International Application No. PCT/US2024/020215, filed on Mar. 15, 2024, which claims priority to, and the benefit of, U.S. Provisional Patent Application No. 63/490,962, filed on Mar. 17, 2023, and U.S. Provisional Patent Application No. 63/519,069, filed on Aug. 11, 2023, the entire contents of each of which are incorporated herein by reference in their entireties.

### BACKGROUND

Innate immune responses are mediated by different types of receptors termed pattern-recognition receptors (PRRs). PRRs recognize the presence of pathogen-associated molecular patterns (PAMPs) and damage-associated molecular patterns (DAMPs). Once engaged these receptors trigger the activation of downstream inflammatory pathways that will help resolve injury. However, in many instances this activation can be uncontrolled and leads to disease.

The inflammasomes represent a class of PRRs that are crucial components of the innate immune response. Activation of the inflammasomes trigger a cascade of events that releases IL-1β, IL-18, and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and acquired immune system. Thus, inflammasome activation is a major regulatory of the inflammatory cascade.

NLRP3 is the most characterized inflammasome and has been shown to be critical in innate immunity and inflammatory responses. While several other NLR complexes, such as NLRC4, are activated under very specific circumstances, NLRP3 can be activated by numerous stimuli and should be seen as a sensor of intracellular homeostatic imbalance. Therefore, its precise functioning is essential. In addition to playing a role in host immune defense, dysregulation of NLRP3 has been linked to the pathogenesis of many inflammatory disorders. These include genetic diseases such as cryopyrin-associated periodic syndromes (CAPS) which is caused by gain-of-function mutations in the NLRP3 gene, as well as many prevalent neurologic and systemic diseases. Importantly, NLRP3 hyperactivation has been demonstrated pre-clinically to play a critical role in a plethora of inflammatory and degenerative diseases including, NASH, atherosclerosis and other cardiovascular diseases, Alzheimer's disease, Parkinson's disease, diabetes, gout, and numerous other autoinflammatory diseases. See, e.g., Li et al., European Journal of Pharmacology (2022) 928:175091; Nguyen et al., Journal of Parkinson's Disease (2022) 12:2117-2133; Su et al., Current Medicinal Chemistry (2021) 28:569-582; Zahid et al., Frontiers in Immunology (2019) 10:2538. Thus, there is an unmet need in the field to develop small molecules for modulating NLRP3 activity to treat various diseases and disorders.

### SUMMARY

Provided herein are NLRP3 inhibitors of Formula (I-B):

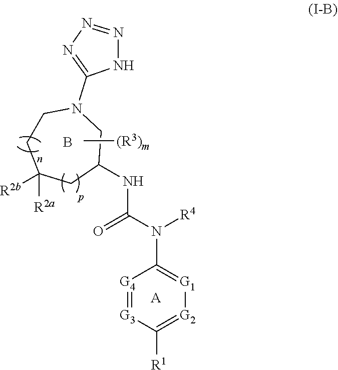

and pharmaceutically acceptable salts and tautomers thereof, wherein Ring A, Ring B, R¹, R^{2a}, R^{2b}, R³, R⁴, m, n, and p are described herein.

Further provided are methods of preparation, methods of treatment and prevention, and pharmaceutical compositions comprising same.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds described herein can additionally encompass individual isomers substantially free of other isomers, and alternatively, as mixtures of various isomers.

For example, compounds described herein may be referred to as “Rac-X”, which, for purposes of the Examples, including the data provided in the Assay Methods section, signifies a mixture of 2 or more stereoisomers, e.g., Compounds X′ X″, X″, X″″, XA, XB, XC, XD, XE, XF, XG, and/or XH. For purposes of claiming a “Rac-X” molecule, the claim may encompass a racemic composition of matter, but also may encompass an enantiomerically enriched composition of matter, e.g., enriched in one stereoisomer over others that may have been generated. For example, a claim may encompass a pharmaceutical composition comprising a “Rac-X” compound, or pharmaceutically acceptable salt thereof, and a pharmaceutically acceptable excipient, wherein the “Rac-X” compound is >80%, >85%, >90%, >95%, or >99% enantiomerically enriched.

Unless otherwise stated, compounds described herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (—CH₃, C₁), ethyl (—CH₂CH₃, C₂), n-propyl (C₃), isopropyl (C₃), n-butyl (C₄), tert-butyl (C₄), sec-butyl (C₄), iso-butyl (C₄), n-pentyl (C), 3-pentanyl (C₅), amyl (C₅), neopentyl (C₅), 3-methyl-2-butanyl (C₅), tertiary amyl (C₅), and n-hexyl (C₆).

“Haloalkyl” refers to a substituted alkyl group, as defined herein, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl group has 1 to 6 carbon atoms (“C₁₋₆ haloalkyl”). In some embodiments, the haloalkyl group has 1 to 5 carbon atoms (“C₁₋₅ haloalkyl”). In some embodiments, the haloalkyl group has 1 to 4 carbon atoms (“C₁₋₄ haloalkyl”). In some embodiments, the haloalkyl group has 1 to 3 carbon atoms (“C₁₋₃ haloalkyl”). In some embodiments, the haloalkyl group has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

“Carbocyclyl” or “carbocyclic” refers to a radical of a non-aromatic cyclic hydrocarbon group having from 3 to 4 ring carbon atoms (“C₃₋₄ carbocyclyl”) and zero heteroatoms in the non-aromatic ring system. In some embodiments, a carbocyclyl group has 3 ring carbon atoms (“C₃ carbocyclyl”). In some embodiments, a carbocyclyl group has 4 ring carbon atoms (“C₄ carbocyclyl”). Exemplary C₃₋₄ carbocyclyl groups include, without limitation, cyclopropyl (C₃), cyclopropenyl (C₃), cyclobutyl (C₄), and cyclobutenyl (C₄).

“C₃-C₄ carbocyclyl-C₁₋₃ alkyl” refers to a C₃-C₄ carbocyclic group, as defined herein, attached to an C₁₋₃ alkyl group, as defined herein, wherein the point of attachment to the parent molecule is on the alkyl group.

“Heterocyclyl” or “heterocyclic” refers to a radical of a 3- to 4-membered non-aromatic ring system having ring carbon atoms and 1 ring heteroatom, wherein each heteroatom is independently selected from nitrogen, oxygen, and sulfur (“3-4 membered heterocyclyl”). In heterocyclyl groups that contain one nitrogen atom, the point of attachment can be a carbon or nitrogen atom, as valency permits. Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl.

“Heteroaryl” refers to a radical of a 5-membered monocyclic aromatic ring system having ring carbon atoms and 1-2 ring heteroatoms provided in the aromatic ring system, wherein each ring heteroatom is independently selected from nitrogen, oxygen, and sulfur (“5-membered heteroaryl”). In heteroaryl groups that contain one or more nitrogen atoms, the point of attachment can be a carbon or nitrogen atom, as valency permits. Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl.

“Halo” or “halogen” refers to fluorine (fluoro, —F), chlorine (chloro, —Cl), bromine (bromo, —Br), or iodine (iodo, —I) radicals.

Affixing the suffix “-ene” to a group indicates the group is a divalent moiety, e.g., alkylene is the divalent moiety of alkyl, and haloalkylene is the divalent moiety of haloalkyl. By way of example, a C₁₋₃ alkylene, which may be linear or branched, include, but are not limited to, —CH₂—, —CH(CH₃)—, —C(CH₃)₂—, —CH₂CH₂—, —CH₂CH(CH₃)—, —CH₂C(CH₃)₂—, and —CH₂CH₂CH₂—.

An alkylene or haloalkylene “bridging group” refers to a group where the two ends of the divalent moiety are attached to different carbon atoms which are not vicinal (next to) each other. Exemplary bridging groups include methylene (—CH₂—), ethylene (—CH₂CH₂—), propylene (—CH₂CH₂CH₂—), and their corresponding halogenated (haloalkylene) groups. For clarity, the phrase “two R³ groups, together with the atoms to which they are attached, may be joined to form a C₁₋₃alkylene bridging group or C₁₋₃haloalkylene bridging group” is used interchangeably herein with the phrase “two R³ groups may be joined to form a C₁₋₃alkylene bridging group or C₁₋₃haloalkylene bridging group between the two atoms to which they are attached”; both phrases mean there are two non-vicinal R³ groups (attached to two carbon atoms of Ring B) which are joined to form a C₁₋₃alkylene bridging group or C₁₋₃haloalkylene bridging group on Ring B. An example of this bridging group is variable L of the compound of Formula (I-B-Bridge).

It is understood herein that compounds of Formula (I-B) and pharmaceutically acceptable salts thereof, each of which contain a terminal tetrazolyl group, may exist as a mixture of tautomeric isomers e.g.,

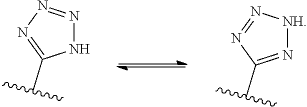 [SMILES: *c1nnn[nH]1 ; *c1nn[nH]n1]

Amino and oxygen protecting groups are described in detail in Protecting Groups in Organic Synthesis, T. W. Greene and P. G. M. Wuts, 3^{rd} edition, John Wiley & Sons, 1999.

Exemplary oxygen (hydroxyl) protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), tetrahydrofuranyl, benzyl (Bn), trimethylsilyl (TMS), triethylsilyl (TES), triisopropylsilyl (TIPS), t-butyldimethylsilyl (TBDMS), benzoylformate, acetate, chloroacetate, dichloroacetate, trichloroacetate, trifluoroacetate, methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), allyl carbonate, t-butyl carbonate (BOC), methanesulfonate (mesylate), benzylsulfonate, and tosylate (Ts).

Exemplary amino protecting groups include, but are not limited to, those that protect the amine as an amide, such as formamide, acetamide, chloroacetamide, trichloroacetamide, trifluoroacetamide, and phenylacetamide; protect the amine as a carbamate, such as methyl carbamate, ethyl carbamate, 9-fluorenylmethyl carbamate (Fmoc), t-butyl carbamate (BOC), 1-adamantyl carbamate (Adoc), and benzyl carbamate (Cbz); and protect the amine as a sulfonamide such as p-toluenesulfonamide (Ts), benzenesulfonamide, methanesulfonamide (Ms), and benzylsulfonamide.

Salts, pharmaceutically acceptable salts, and free bases of compounds of Formula (I-B) are contemplated herein.

“Salt” refers to any and all salts.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Examples of pharmaceutically acceptable acid addition salts include, but are not limited to, salts formed from inorganic acids, such as hydrochloric acid, hydrobromic acid, phosphoric acid, sulfuric acid and perchloric acid salts, or salts formed from organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “free base” refers to a neutral non-ionized form of a compound which is not a salt or pharmaceutically acceptable salt.

A “patient” or “subject” is used interchangeably herein, and refers to a mammal, e.g., a human, mouse, rat, guinea pig, dog, cat, horse, cow, pig, or non-human primate, such as a monkey, chimpanzee, baboon, or rhesus. In certain embodiments, the patient or subject is human.

“Effective amount” or “therapeutically effective amount” are used interchangeably herein, and refer to an amount of a compound, or a pharmaceutically acceptable salt or tautomer thereof, sufficient to provide a therapeutic benefit in the treatment of a disease or disorder, or to delay or minimize one or more symptoms associated with the disease or disorder in a subject in need thereof. An effective amount can encompass an amount that improves overall therapy, reduces or avoids symptoms or causes of disease or disorder, or enhances the therapeutic efficacy of another therapeutic agent. The effective amount of a compound, or a pharmaceutically acceptable salt or tautomer thereof, may vary depending on such factors as the desired biological endpoint, the pharmacokinetics of the compound, the disease being treated, the mode of administration, and the age, health, and condition of the subject.

“Disease” or “disorder” are used interchangeably herein.

“Treating” or “treat” or “treatment” describes the management and care of a subject in need thereof, for the purpose of combating a disease or disorder in the subject, and includes the administration of a compound, or a pharmaceutically acceptable salt or tautomer thereof, as described herein, to alleviate the symptoms or complications of a disease or disorder, or to eliminate the disease or disorder. The term “treat” can also include treatment of a cell in vitro or treatment of an animal model (in vivo). It is to be appreciated that references to “treating” or “treatment” include the alleviation of established symptoms of a disease or disorder in a subject in need thereof, and therefore includes: (1) delaying the appearance of at least one clinical or subclinical symptom of the disease or disorder developing in a subject that is afflicted with the disease or disorder, (2) arresting, reducing or delaying the continued development of the disease or a relapse thereof in a subject (e.g., in case of maintenance treatment) or at least one clinical or subclinical symptom thereof, or (3) relieving or attenuating the disease in a subject, i.e., causing regression of the disease or disorder or at least one of its clinical or subclinical symptoms.

As used herein, the term “preventing,” “prevent,” or “protecting against” describes the management and care of a subject in need thereof that may have or has a predisposition for the disease or disorder but has not yet experienced or displayed symptoms or complications of a disease or disorder (e.g., clinical or subclinical symptoms of the disease or disorder), for the purpose of preventing the appearance of said symptoms or complications of the disease or disorder in the subject, and includes the administration of a compound, or a pharmaceutically acceptable salt or tautomer thereof, as described herein.

“Inhibition”, “inhibiting”, “inhibit” and “inhibitor”, and the like, refer to the ability of a compound, or a pharmaceutically acceptable salt or tautomer thereof, to reduce, slow, halt or prevent activity of a particular biological process (e.g., NLRP3 activity) in a cell relative to vehicle.

The phrase “at least one” refers to one instance or more than one instance.

The articles “a” and “an” are used in this disclosure to refer to one or more than one (i.e., to at least one) of the grammatical object of the article.

The term “and/or” is used in this disclosure to mean either “and” or “or” unless indicated otherwise.

### DETAILED DESCRIPTION OF SOME EMBODIMENTS

### (i) Compounds

Provided herein are compounds of Formula (I-B):

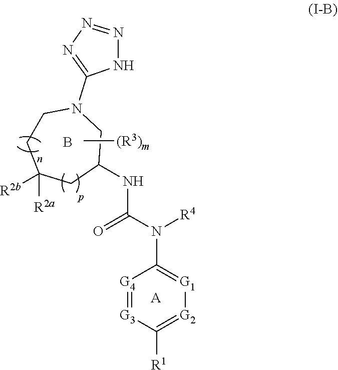

and pharmaceutically acceptable salts and tautomers thereof;
wherein:
Ring A is a ring system wherein:
   G₁ is CR^{G1} or N; G₂ is CR^{G2} or N; G₃ is CR^{G3} or N; and G₄ is CR^{G4} or N; provided no more than two of G₁, G₂, G₃, and G₄ are N;
   R¹ is halo, C₁₋₆ alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆alkyl, C₁₋₆haloalkyl, —OR^{G5}, —SR^{G5}, or —N(R^{G5})₂,
   or R¹ and G₂, together with the atoms to which they are attached, are joined to form a 5-membered heteroaryl ring independently substituted with 0, 1, 2, or 3 R^{G7};
   R^{G1}, R^{G2}, R^{G3}, and R^{G4} are each independently selected from the group consisting of hydrogen, halo, C₁₋₆ alkyl, C₁₋₆ haloalkyl, and —OR^{G6};
   R^{G5} and R^{G6} are each independently hydrogen, C₁₋₆ alkyl, or C₁₋₆ haloalkyl; and
   each instance of R^{G7} is independently halo, C₁₋₆ alkyl, C₁₋₆ haloalkyl, —OR^{G5}, —SR^{G5}, and —N(R^{G5})₂; and
Ring B is a ring system wherein:
   n is 0 or 1;
   p is 1 or 2;
   m is 0, 1, 2, or 3;
   each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo;
   each instance of R³ is independently halo, C₁₋₆ alkyl or C₁₋₆ haloalkyl, or two R³ groups are joined to form a C₁₋₃ alkylene bridging group or C₁₋₃ haloalkylene bridging group; and
   R⁴ is hydrogen, C₁₋₃ alkyl, C₃-C₄ carbocyclyl, or C₃-C₄ carbocyclyl-C₁₋₃ alkyl-, wherein the alkyl and carbocyclyl are each independently substituted with 0, 1, 2, 3, 4, 5, or 6 halo, and wherein the carbocyclyl is further independently substituted with 0, 1, or 2 C₁₋₃ alkyl or C₁₋₃ haloalkyl.

In some embodiments of Formula (I-B), the compound is of Formula:

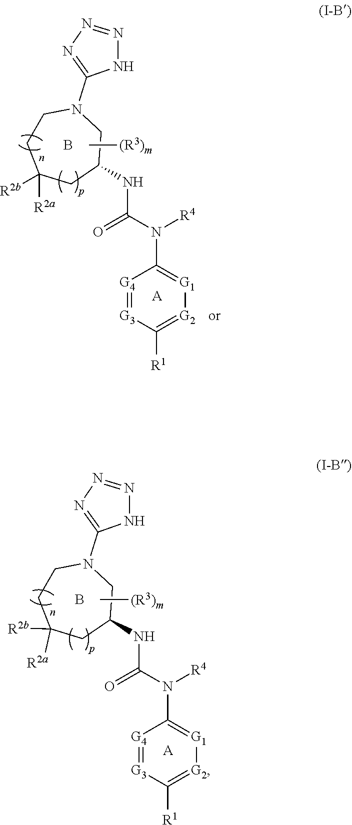

or a pharmaceutically acceptable salt or tautomer thereof.

In some embodiments of Formula (I-B), the compound is of Formula:

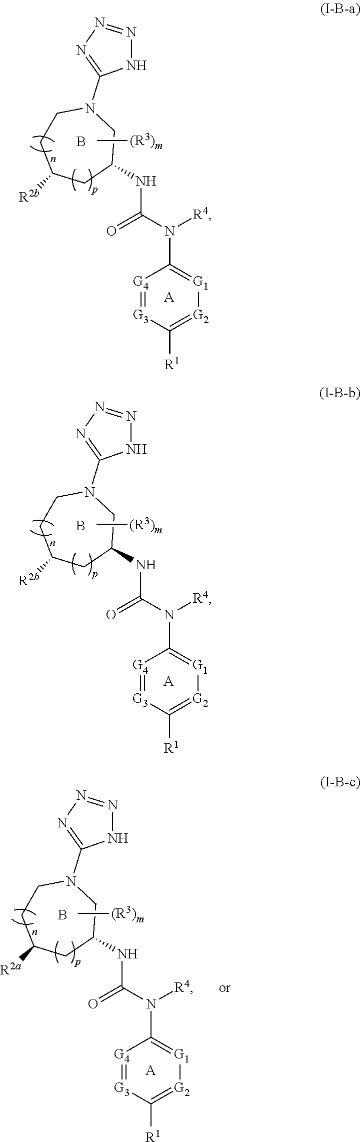

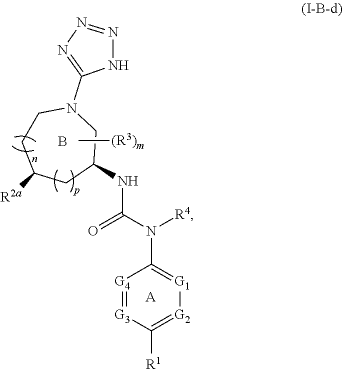

or a pharmaceutically acceptable salt or tautomer thereof.

In some embodiments of Formula (I-B), the compound is of Formula:

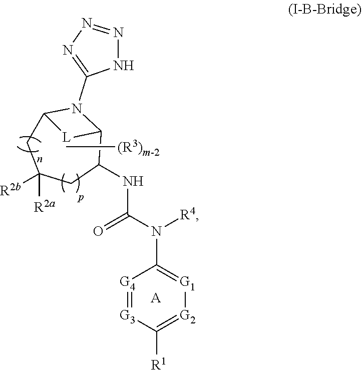

or a pharmaceutically acceptable salt or tautomer thereof, wherein L is a C₁₋₃ alkylene bridging group or a C₁₋₃ haloalkylene bridging group. In some embodiments, L is —CH₂CH₂—.

In some embodiments of Formula (I-B), the compound is of Formula:

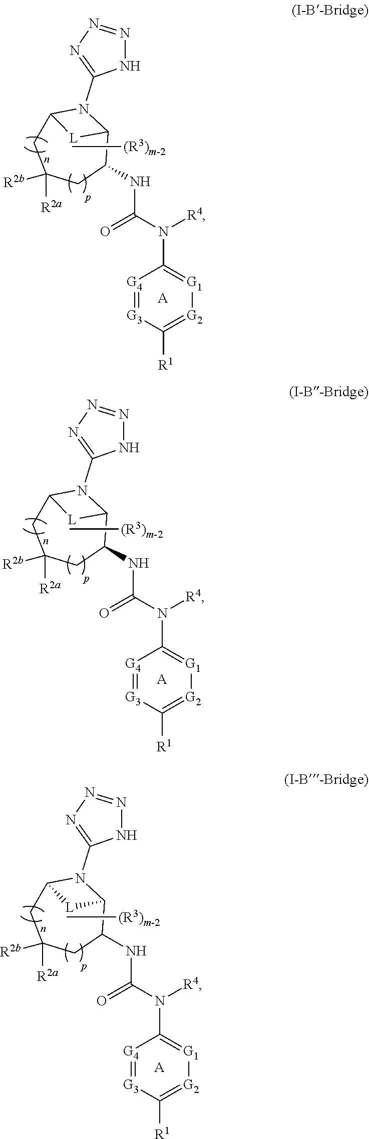

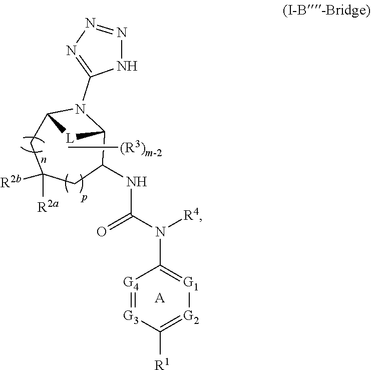

or a pharmaceutically acceptable salt or tautomer thereof, wherein L is a C₁₋₃ alkylene bridging group or a C₁₋₃ haloalkylene bridging group. In some embodiments, L is —CH₂CH₂—.

In some embodiments of Formula (I-B), the compound is of Formula:

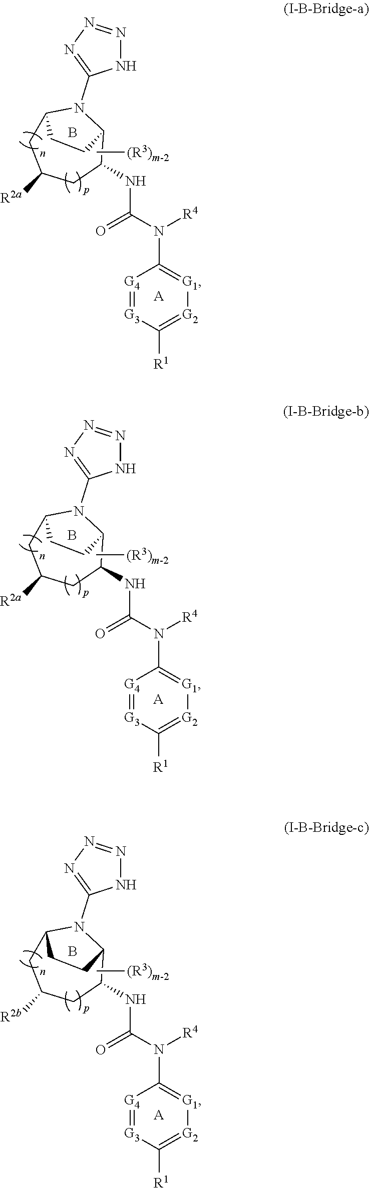

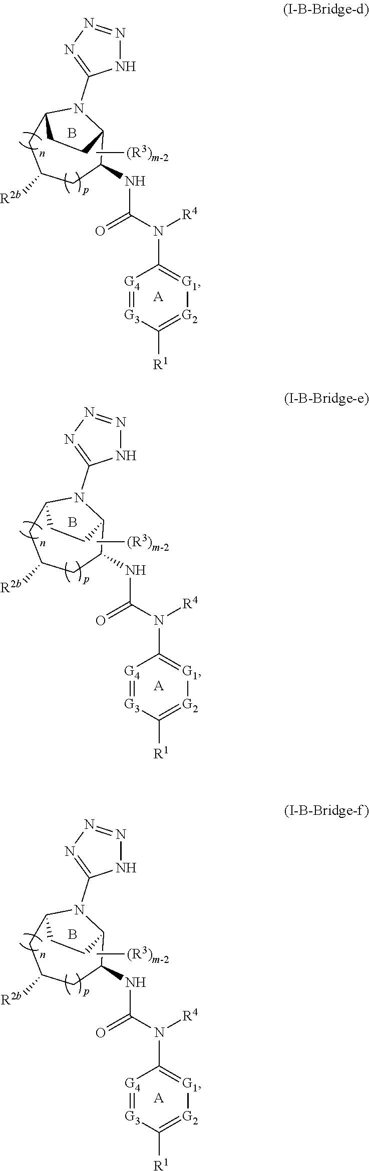

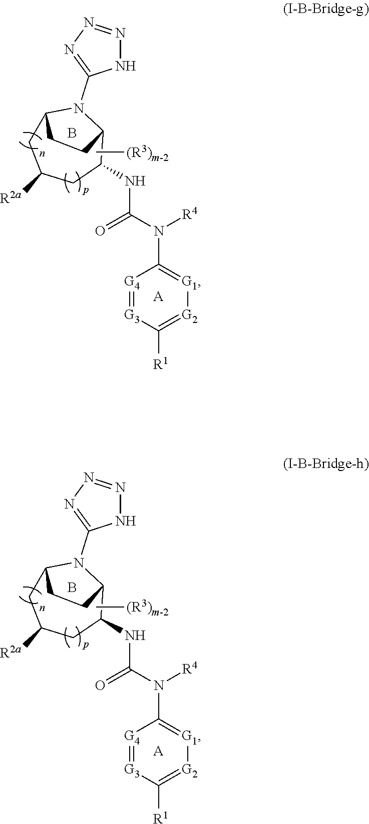

or a pharmaceutically acceptable salt or tautomer thereof.

Additional embodiments are further described below and herein.

(a) Ring A, G₁, G₂, G₃, G₄, R¹, R^{G1}, R^{G2}, R^{G3}, R^{G4}, R^{G5}, R^{G6}, and R^{G7}

As generally described herein, G₁ is CR^{G1} or N; G₂ is CR^{G2} or N; G₃ is CR^{G3} or N; and G₄ is CR^{G4} or N; provided no more than two of G₁, G₂, G₃, and G₄ are N.

In some embodiments, G₁ is CR^{G1}. In some embodiments, G₁ is N.

In some embodiments, G₂ is CR^{G2}. In some embodiments, G₂ is N.

In some embodiments, G₃ is CR^{G3}. In some embodiments, G₃ is N.

In some embodiments, G₄ is CR^{G4}. In some embodiments, G₄ is N.

In some embodiments, G₁ is CR^{G1}; G₂ is CR^{G2}; G₃ is CR^{G3}; and G₄ is CR^{G4}.

In some embodiments, at least one of G₁, G₂, G₃, and G₄ is N.

In some embodiments, G₁ is CR^{G1}; G₂ is CR^{G2}; G₃ is CR^{G3}; and G₄ is N. In some embodiments, G₁ is CR^{G1}; G₂ is CR^{G2}; G₃ is N; and G₄ is CR^{G4}. In some embodiments, G₁ is CR^{G1}; G₂ is N; G₃ is CR^{G3}; and G₄ is CR^{G4}. In some embodiments, G₁ is N; G₂ is CR^{G2}; G₃ is CR^{G3}; and G₄ is CR^{G4}.

In some embodiments, two of G₁, G₂, G₃, and G₄ is N.

For example, in some embodiments, G₁ is CR^{G1}; G₂ is CR^{G2}; G₃ is N; and G₄ is N. In some embodiments, G₁ is CR^{G1}; G₂ is N; G₃ is CR^{G3}; and G₄ is N. In some embodiments, G₁ is N; G₂ is CR^{G2}; G₃ is CR^{G3}; and G₄ is N. In some embodiments, G₁ is N; G₂ is N; G₃ is CR^{G3}; and G₄ is CR^{G4}. In some embodiments, G₁ is N; G₂ is CR^{G2}; G₃ is N; and G₄ is CR^{G4}. In some embodiments, G₁ is CR^{G1}; G₂ is N; G₃ is N; and G₄ is CR^{G4}.

In some embodiments, G₁ is CR^{G1}, G₂ is CR^{G2}, G₃ is CR^{G3}, and G₄ is CR^{G4}; G₁ is N, G₂ is CR^{G2}, G₃ is CR^{G3}, and G₄ is CR^{G4}; G₁ is CR^{G1}, G₂ is N, G₃ is CR^{G3}, and G₄ is CR^{G4}; G₁ is CR^{G1}, G₂ is CR^{G2}, G₃ is N, and G₄ is CR^{G4}; G₁ is CR^{G1}, G₂ is CR^{G2}, G₃ is CR^{G3}, and G₄ is N; G₁ is N, G₂ is CR^{G2}, G₃ is CR^{G3} and G₄ is N; G₁ is N, G₂ is CR^{G2}, G₃ is N, and G₄ is CR^{G4}; G₁ is CR^{G1}, G₂ is N, G₃ is N, and G₄ is CR^{G4}; or G₁ is CR^{G1}, G₂ is CR^{G2}, G₃ is N, and G₄ is N.

In some embodiments, G₁ is CH, G₂ is CH, G₃ is CH, and G₄ is CH; G₁ is N, G₂ is CH, G₃ is CH, and G₄ is CH; G₁ is CH, G₂ is N, G₃ is CH, and G₄ is CH; G₁ is N, G₂ is CH, G₃ is CH, and G₄ is N; G₁ is N, G₂ is CH, G₃ is N, and G₄ is CH; or G₁ is CH, G₂ is CH, G₃ is N, and G₄ is N.

As generally described herein, R¹ is halo, C₁₋₆alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆ haloalkyl, —OR^{G5}, —SR^{G5}, or —N(R^{G5})₂, or R¹ and G₂, together with the atoms to which they are attached, are joined to form a 5-membered heteroaryl ring independently substituted with 0, 1, 2, or 3 R^{G7} selected from the group consisting of halo, C₁₋₆ alkyl, C₁₋₆ haloalkyl, —OR^{G5}, —SR^{G5}, and —N(R^{G5})₂.

In some embodiments, R¹ is halo.

In some embodiments, R¹ is F, Cl, Br, or I. In some embodiments, R¹ is F, Cl, or Br. In some embodiments, R¹ is F or Cl.

In some embodiments, R¹ is F. In some embodiments, R¹ is Cl. In some embodiments, R¹ is Br. In some embodiments, R¹ is I.

In some embodiments, R¹ is C₁₋₆ alkyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₁₋₆ alkyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is unsubstituted C₁₋₆ alkyl.

In some embodiments, R¹ is methyl. In some embodiments, R¹ is ethyl. In some embodiments, R¹ is propyl. In some embodiments, R¹ is butyl. In some embodiments, R¹ is pentyl. In some embodiments, R¹ is hexyl. In some embodiments, R¹ is isopropyl. In some embodiments, R¹ is isobutyl. In some embodiments, R¹ is isopentyl. In some embodiments, R¹ is isohexyl. In some embodiments, R¹ is secbutyl. In some embodiments, R¹ is secpentyl. In some embodiments, R¹ is sechexyl. In some embodiments, R¹ is tertbutyl.

In some embodiments, R¹ is C₁₋₆ haloalkyl.

In some embodiments, R¹ is halomethyl. In some embodiments, R¹ is haloethyl. In some embodiments, R¹ is halopropyl. In some embodiments, R¹ is halobutyl. In some embodiments, R¹ is halopentyl. In some embodiments, R¹ is halohexyl.

In some embodiments, R¹ is —OR^{G5}.

In some embodiments, R¹ is —SR^{G5}.

In some embodiments, R¹ is —N(R^{G5})₂.

In some embodiments, R¹ is C₃-C₄ carbocyclyl or 3-4 membered heterocyclyl, wherein the carbocyclyl or heterocyclyl are independently substituted with 0, 1, 2, or 3 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 0, 1, 2, or 3 halo, C₁₋₆alkyl, C₁₋₆haloalkyl, —OR^{G5}, —SR^{G5}, or —N(R^{G5})₂.

In some embodiments, R¹ is C₃ carbocyclyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₄ carbocyclyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₃-C₄ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is C₄ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is unsubstituted C₃-C₄ carbocyclyl.

In some embodiments, R¹ is unsubstituted C₃ carbocyclyl. In some embodiments, R¹ is unsubstituted C₄ carbocyclyl.

In some embodiments, R¹ is C₃-C₄ carbocyclyl substituted with 1 halo, C₁₋₆alkyl, C₁₋₆haloalkyl, —OR^{G5}, —SR^{G5}, or —N(RGS)₂.

In some embodiments, R¹ is C₃ carbocyclyl substituted with 1 halo, C₁₋₆ alkyl, C₁₋₆ haloalkyl, —OR^{G5}, —SR^{G5}, or —N(R^{G5})₂. In some embodiments, R¹ is C₄ carbocyclyl substituted with 1 halo, C₁₋₆ alkyl, C₁₋₆haloalkyl, —OR^{G5}, —SR^{G5}, or —N(R^{G5})₂.

In some embodiments, R¹ is C₃-C₄ carbocyclyl independently substituted with 2 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 2 halo, C₁₋₆ alkyl, C₁₋₆ haloalkyl, —OR^{G5}, —SR^{G5}, or —N(R^{G5})₂. In some embodiments, R¹ is C₄ carbocyclyl independently substituted with 2 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 3 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 3 halo, C₁₋₆ alkyl, C₁₋₆ haloalkyl, —OR^{G5}, —SR^{G5}, or —N(R^{G5})₂. In some embodiments, R¹ is C₄ carbocyclyl independently substituted with 3 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is halo.

In some embodiments, R¹ is C₃ carbocyclyl substituted with at least one halo. In some embodiments, R¹ is C₄ carbocyclyl substituted with at least one halo.

In some embodiments, R¹ is C₃-C₄ carbocyclyl substituted with at least one of F, Cl, Br, or I. In some embodiments, R¹ is C₃-C₄ carbocyclyl substituted with at least one of F, Cl, or Br. In some embodiments, R¹ is C₃-C₄ carbocyclyl substituted with at least one of F or Cl.

In some embodiments, R¹ is C₃ carbocyclyl substituted with at least one of F, Cl, Br, or I. In some embodiments, R¹ is C₃ carbocyclyl substituted with at least one of F, Cl, or Br. In some embodiments, R¹ is C₃ carbocyclyl substituted with at least one of F or Cl.

In some embodiments, R¹ is C₄ carbocyclyl substituted with at least one of F, Cl, Br, or I. In some embodiments, R¹ is C₄ carbocyclyl substituted with at least one of F, Cl, or Br. In some embodiments, R¹ is C₄ carbocyclyl substituted with at least one of F or Cl.

In some embodiments, R¹ is C₃-C₄ carbocyclyl substituted with at least one F. In some embodiments, R¹ is C₃-C₄ carbocyclyl substituted with at least one Cl. In some embodiments, R¹ is C₃-C₄ carbocyclyl substituted with at least one Br. In some embodiments, R¹ is C₃-C₄ carbocyclyl substituted with at least one I.

In some embodiments, R¹ is C₃ carbocyclyl substituted with at least one F. In some embodiments, R¹ is C₃ carbocyclyl substituted with at least one Cl. In some embodiments, R¹ is C₃ carbocyclyl substituted with at least one Br. In some embodiments, R¹ is C₃ carbocyclyl substituted with at least one I.

In some embodiments, R¹ is C₄ carbocyclyl substituted with at least one F. In some embodiments, R¹ is C₄ carbocyclyl substituted with at least one Cl. In some embodiments, R¹ is C₄ carbocyclyl substituted with at least one Br. In some embodiments, R¹ is C₄ carbocyclyl substituted with at least one I.

In some embodiments, R¹ is C₃-C₄ carbocyclyl substituted with at least one C₁₋₆ alkyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ alkyl.

In some embodiments, R¹ is C₃-C₄ carbocyclyl substituted with at least one methyl. In some embodiments, R¹ is C₃-C₄ carbocyclyl substituted with at least one ethyl. In some embodiments, R¹ is C₃-C₄ carbocyclyl substituted with at least one propyl. In some embodiments, R¹ is C₃-C₄ carbocyclyl substituted with at least one butyl. In some embodiments, R¹ is C₃-C₄ carbocyclyl substituted with at least one pentyl. In some embodiments, R¹ is C₃-C₄ carbocyclyl substituted with at least one hexyl. In some embodiments, R¹ is C₃-C₄ carbocyclyl substituted with at least one isopropyl. In some embodiments, R¹ is C₃-C₄ carbocyclyl substituted with at least one isobutyl. In some embodiments, R¹ is C₃-C₄ carbocyclyl substituted with at least one isopentyl. In some embodiments, R¹ is C₃-C₄ carbocyclyl substituted with at least one isohexyl. In some embodiments, R¹ is C₃-C₄ carbocyclyl substituted with at least one secbutyl. In some embodiments, R¹ is C₃-C₄ carbocyclyl substituted with at least one secpentyl. In some embodiments, R¹ is C₃-C₄ carbocyclyl substituted with at least one sechexyl. In some embodiments, R¹ is C₃-C₄ carbocyclyl substituted with at least one tertbutyl.

In some embodiments, R¹ is C₃ carbocyclyl substituted with at least one methyl. In some embodiments, R¹ is C₃ carbocyclyl substituted with at least one ethyl. In some embodiments, R¹ is C₃ carbocyclyl substituted with at least one propyl. In some embodiments, R¹ is C₃ carbocyclyl substituted with at least one butyl. In some embodiments, R¹ is C₃ carbocyclyl substituted with at least one pentyl. In some embodiments, R¹ is C₃ carbocyclyl substituted with at least one hexyl. In some embodiments, R¹ is C₃ carbocyclyl substituted with at least one isopropyl. In some embodiments, R¹ is C₃ carbocyclyl substituted with at least one isobutyl. In some embodiments, R¹ is C₃ carbocyclyl substituted with at least one isopentyl. In some embodiments, R¹ is C₃ carbocyclyl substituted with at least one isohexyl. In some embodiments, R¹ is C₃ carbocyclyl substituted with at least one secbutyl. In some embodiments, R¹ is C₃ carbocyclyl substituted with at least one secpentyl. In some embodiments, R¹ is C₃ carbocyclyl substituted with at least one sechexyl. In some embodiments, R¹ is C₃ carbocyclyl substituted with at least one tertbutyl.

In some embodiments, R¹ is C₄ carbocyclyl substituted with at least one methyl. In some embodiments, R¹ is C₄ carbocyclyl substituted with at least one ethyl. In some embodiments, R¹ is C₄ carbocyclyl substituted with at least one propyl. In some embodiments, R¹ is C₄ carbocyclyl substituted with at least one butyl. In some embodiments, R¹ is C₄ carbocyclyl substituted with at least one pentyl. In some embodiments, R¹ is C₄ carbocyclyl substituted with at least one hexyl. In some embodiments, R¹ is C₄ carbocyclyl substituted with at least one isopropyl. In some embodiments, R¹ is C₄ carbocyclyl substituted with at least one isobutyl. In some embodiments, R¹ is C₄ carbocyclyl substituted with at least one isopentyl. In some embodiments, R¹ is C₄ carbocyclyl substituted with at least one isohexyl. In some embodiments, R¹ is C₄ carbocyclyl substituted with at least one secbutyl. In some embodiments, R¹ is C₄ carbocyclyl substituted with at least one secpentyl. In some embodiments, R¹ is C₄ carbocyclyl substituted with at least one sechexyl. In some embodiments, R¹ is C₄ carbocyclyl substituted with at least one tertbutyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ haloalkyl.

In some embodiments, R¹ is C₃ carbocyclyl substituted with at least one C₁₋₆ haloalkyl. In some embodiments, R¹ is C₄ carbocyclyl substituted with at least one C₁₋₆ haloalkyl.

In some embodiments, R¹ is C₃-C₄ carbocyclyl substituted with at least one halomethyl. In some embodiments, R¹ is C₃-C₄ carbocyclyl substituted with at least one haloethyl. In some embodiments, R¹ is C₃-C₄ carbocyclyl substituted with at least one halopropyl. In some embodiments, R¹ is C₃-C₄ carbocyclyl substituted with at least one halobutyl. In some embodiments, R¹ is C₃-C₄ carbocyclyl substituted with at least one halopentyl. In some embodiments, R¹ is C₃-C₄ carbocyclyl substituted with at least one halohexyl.

In some embodiments, R¹ is C₃ carbocyclyl substituted with at least one halomethyl. In some embodiments, R¹ is C₃ carbocyclyl substituted with at least one haloethyl. In some embodiments, R¹ is C₃ carbocyclyl substituted with at least one halopropyl. In some embodiments, R¹ is C₃ carbocyclyl substituted with at least one halobutyl. In some embodiments, R¹ is C₃ carbocyclyl substituted with at least one halopentyl. In some embodiments, R¹ is C₃ carbocyclyl substituted with at least one halohexyl.

In some embodiments, R¹ is C₄ carbocyclyl substituted with at least one halomethyl. In some embodiments, R¹ is C₄ carbocyclyl substituted with at least one haloethyl. In some embodiments, R¹ is C₄ carbocyclyl substituted with at least one halopropyl. In some embodiments, R¹ is C₄ carbocyclyl substituted with at least one halobutyl. In some embodiments, R¹ is C₄ carbocyclyl substituted with at least one halopentyl. In some embodiments, R¹ is C₄ carbocyclyl substituted with at least one halohexyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —OR^{G5}.

In some embodiments, R¹ is C₃ carbocyclyl substituted with at least one —OR^{G5}. In some embodiments, R¹ is C₄ carbocyclyl substituted with at least one —OR^{G5}.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —SR^{G5}.

In some embodiments, R¹ is C₃ carbocyclyl substituted with at least one —SR^{G5}. In some embodiments, R¹ is C₄ carbocyclyl substituted with at least one —SR^{G5}.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —N(R^{G5})₂.

In some embodiments, R¹ is C₃ carbocyclyl substituted with at least one —N(R^{G5})₂. In some embodiments, R¹ is C₄ carbocyclyl substituted with at least one —N(R^{G5})₂.

In some embodiments, R¹ is 3-4 membered heterocyclyl independently substituted with 0, 1, 2, or 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 0, 1, 2, or 3 halo, C₁₋₆alkyl, C₁₋₆haloalkyl, —OR^{G5}, —SR^{G5}, or —N(R^{G5})₂. In some embodiments, R¹ is 4-membered heterocyclyl independently substituted with 0, 1, 2, or 3 halo, C₁₋₆ alkyl, C₁₋₆ haloalkyl, —OR^{G5}, —SR^{G5} or —N(R^{G5})₂.

In some embodiments, R¹ is 3-4 membered heterocyclyl independently substituted with 1, 2, or 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 1, 2, or 3 halo, C₁₋₆alkyl, C₁₋₆haloalkyl, —OR^{G5}, —SR^{G5}, or —N(R^{G5})₂. In some embodiments, R¹ is 4-membered heterocyclyl independently substituted with 1, 2, or 3 halo, C₁₋₆ alkyl, C₁₋₆ haloalkyl, —OR^{G5}, —SR^{G5} or —N(R^{G5})₂.

In some embodiments, R¹ is unsubstituted 3-4 membered heterocyclyl.

In some embodiments, R¹ is unsubstituted 3-membered heterocyclyl. In some embodiments, R¹ is unsubstituted 4-membered heterocyclyl.

In some embodiments, R¹ is 3-4 membered heterocyclyl independently substituted with 1 halo, C₁₋₆alkyl, C₁₋₆haloalkyl, —OR^{G5}, —SR^{G5}, or —N(R^{G5})₂.

In some embodiments, R¹ is 3-membered heterocyclyl substituted with 1 halo, C₁₋₆ alkyl, C₁₋₆ haloalkyl, —OR^{G5}, —SR^{G5}, or —N(R^{G5})₂. In some embodiments, R¹ is 4-membered heterocyclyl substituted with 1 halo, C₁₋₆alkyl, C₁₋₆haloalkyl, —OR^{G5}, —SR^{G5}, or —N(R^{G5})₂.

In some embodiments, R¹ is 3-4 membered heterocyclyl independently substituted with 2 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 2 halo, C₁₋₆ alkyl, C₁₋₆ haloalkyl, —OR^{G5}, —SR^{G5}, or —N(R^{G5})₂. In some embodiments, R¹ is 4-membered heterocyclyl independently substituted with 2 halo, C₁₋₆alkyl, C₁₋₆haloalkyl, —OR^{G5}, —SR^{G5}, or —N(R^{G5})₂.

In some embodiments, R¹ is 3-4 membered heterocyclyl independently substituted with 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 3 halo, C₁₋₆ alkyl, C₁₋₆ haloalkyl, —OR^{G5}, —SR^{G5}, or —N(R^{G5})₂. In some embodiments, R¹ is 4-membered heterocyclyl independently substituted with 3 halo, C₁₋₆alkyl, C₁₋₆haloalkyl, —OR^{G5}, —SR^{G5}, or —N(R^{G5})₂.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is halo.

In some embodiments, R¹ is 3-membered heterocyclyl substituted with at least one halo. In some embodiments, R¹ is 4-membered heterocyclyl substituted with at least one halo.

In some embodiments, R¹ is 3-4 membered heterocyclyl substituted with at least one of F, Cl, Br, or I. In some embodiments, R¹ is 3-4 membered heterocyclyl substituted with at least one of F, Cl, or Br. In some embodiments, R¹ is 3-4 membered heterocyclyl substituted with at least one of F or Cl.

In some embodiments, R¹ is 3-membered heterocyclyl substituted with at least one of F, Cl, Br, or I. In some embodiments, R¹ is 3-membered heterocyclyl substituted with at least one of F, Cl, or Br. In some embodiments, R¹ is 3-membered heterocyclyl substituted with at least one of F or Cl.

In some embodiments, R¹ is 4-membered heterocyclyl substituted with at least one of F, Cl, Br, or I. In some embodiments, R¹ is 4-membered heterocyclyl substituted with at least one of F, Cl, or Br. In some embodiments, R¹ is 4-membered heterocyclyl substituted with at least one of F or Cl.

In some embodiments, R¹ is 3-4 membered heterocyclyl substituted with at least one F. In some embodiments, R¹ is 3-4 membered heterocyclyl substituted with at least one Cl. In some embodiments, R¹ is 3-4 membered heterocyclyl substituted with at least one Br. In some embodiments, R¹ is 3-4 membered heterocyclyl substituted with at least one I.

In some embodiments, R¹ is 3-membered heterocyclyl substituted with at least one F. In some embodiments, R¹ is 3-membered heterocyclyl substituted with at least one Cl. In some embodiments, R¹ is 3-membered heterocyclyl substituted with at least one Br. In some embodiments, R¹ is 3-membered heterocyclyl substituted with at least one I.

In some embodiments, R¹ is 4-membered heterocyclyl substituted with at least one F. In some embodiments, R¹ is 4-membered heterocyclyl substituted with at least one Cl. In some embodiments, R¹ is 4-membered heterocyclyl substituted with at least one Br. In some embodiments, R¹ is 4-membered heterocyclyl substituted with at least one I.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ alkyl.

In some embodiments, R¹ is 3-membered heterocyclyl substituted with at least one C₁₋₆ alkyl. In some embodiments, R¹ is 4-membered heterocyclyl substituted with at least one C₁₋₆ alkyl.

In some embodiments, R¹ is 3-4 membered heterocyclyl substituted with at least one methyl. In some embodiments, R¹ is 3-4 membered heterocyclyl substituted with at least one ethyl. In some embodiments, R¹ is 3-4 membered heterocyclyl substituted with at least one propyl. In some embodiments, R¹ is 3-4 membered heterocyclyl substituted with at least one butyl. In some embodiments, R¹ is 3-4 membered heterocyclyl substituted with at least one pentyl. In some embodiments, R¹ is 3-4 membered heterocyclyl substituted with at least one hexyl. In some embodiments, R¹ is 3-4 membered heterocyclyl substituted with at least one isopropyl. In some embodiments, R¹ is 3-4 membered heterocyclyl substituted with at least one isobutyl. In some embodiments, R¹ is 3-4 membered heterocyclyl substituted with at least one isopentyl. In some embodiments, R¹ is 3-4 membered heterocyclyl substituted with at least one isohexyl. In some embodiments, R¹ is 3-4 membered heterocyclyl substituted with at least one secbutyl. In some embodiments, R¹ is 3-4 membered heterocyclyl substituted with at least one secpentyl. In some embodiments, R¹ is 3-4 membered heterocyclyl substituted with at least one sechexyl. In some embodiments, R¹ is 3-4 membered heterocyclyl substituted with at least one tertbutyl.

In some embodiments, R¹ is 3-membered heterocyclyl substituted with at least one methyl. In some embodiments, R¹ is 3-membered heterocyclyl substituted with at least one ethyl. In some embodiments, R¹ is 3-membered heterocyclyl substituted with at least one propyl. In some embodiments, R¹ is 3-membered heterocyclyl substituted with at least one butyl. In some embodiments, R¹ is 3-membered heterocyclyl substituted with at least one pentyl. In some embodiments, R¹ is 3-membered heterocyclyl substituted with at least one hexyl. In some embodiments, R¹ is 3-membered heterocyclyl substituted with at least one isopropyl. In some embodiments, R¹ is 3-membered heterocyclyl substituted with at least one isobutyl. In some embodiments, R¹ is 3-membered heterocyclyl substituted with at least one isopentyl. In some embodiments, R¹ is 3-membered heterocyclyl substituted with at least one isohexyl. In some embodiments, R¹ is 3-membered heterocyclyl substituted with at least one secbutyl. In some embodiments, R¹ is 3-membered heterocyclyl substituted with at least one secpentyl. In some embodiments, R¹ is 3-membered heterocyclyl substituted with at least one sechexyl. In some embodiments, R¹ is 3-membered heterocyclyl substituted with at least one tertbutyl.

In some embodiments, R¹ is 4-membered heterocyclyl substituted with at least one methyl. In some embodiments, R¹ is 4-membered heterocyclyl substituted with at least one ethyl. In some embodiments, R¹ is 4-membered heterocyclyl substituted with at least one propyl. In some embodiments, R¹ is 4-membered heterocyclyl substituted with at least one butyl. In some embodiments, R¹ is 4-membered heterocyclyl substituted with at least one pentyl. In some embodiments, R¹ is 4-membered heterocyclyl substituted with at least one hexyl. In some embodiments, R¹ is 4-membered heterocyclyl substituted with at least one isopropyl. In some embodiments, R¹ is 4-membered heterocyclyl substituted with at least one isobutyl. In some embodiments, R¹ is 4-membered heterocyclyl substituted with at least one isopentyl. In some embodiments, R¹ is 4-membered heterocyclyl substituted with at least one isohexyl. In some embodiments, R¹ is 4-membered heterocyclyl substituted with at least one secbutyl. In some embodiments, R¹ is 4-membered heterocyclyl substituted with at least one secpentyl. In some embodiments, R¹ is 4-membered heterocyclyl substituted with at least one sechexyl. In some embodiments, R¹ is 4-membered heterocyclyl substituted with at least one tertbutyl.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ haloalkyl.

In some embodiments, R¹ is 3-membered heterocyclyl substituted with at least one C₁₋₆ haloalkyl. In some embodiments, R¹ is 4-membered heterocyclyl substituted with at least one C₁₋₆ haloalkyl.

In some embodiments, R¹ is 3-4 membered heterocyclyl substituted with at least one halomethyl. In some embodiments, R¹ is 3-4 membered heterocyclyl substituted with at least one haloethyl. In some embodiments, R¹ is 3-4 membered heterocyclyl substituted with at least one halopropyl. In some embodiments, R¹ is 3-4 membered heterocyclyl substituted with at least one halobutyl. In some embodiments, R¹ is 3-4 membered heterocyclyl substituted with at least one halopentyl. In some embodiments, R¹ is 3-4 membered heterocyclyl substituted with at least one halohexyl.

In some embodiments, R¹ is 3-membered heterocyclyl substituted with at least one halomethyl. In some embodiments, R¹ is 3-membered heterocyclyl substituted with at least one haloethyl. In some embodiments, R¹ is 3-membered heterocyclyl substituted with at least one halopropyl. In some embodiments, R¹ is 3-membered heterocyclyl substituted with at least one halobutyl. In some embodiments, R¹ is 3-membered heterocyclyl substituted with at least one halopentyl. In some embodiments, R¹ is 3-membered heterocyclyl substituted with at least one halohexyl.

In some embodiments, R¹ is 4-membered heterocyclyl substituted with at least one halomethyl. In some embodiments, R¹ is 4-membered heterocyclyl substituted with at least one haloethyl. In some embodiments, R¹ is 4-membered heterocyclyl substituted with at least one halopropyl. In some embodiments, R¹ is 4-membered heterocyclyl substituted with at least one halobutyl. In some embodiments, R¹ is 4-membered heterocyclyl substituted with at least one halopentyl. In some embodiments, R¹ is 4-membered heterocyclyl substituted with at least one halohexyl.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —OR^{G5}.

In some embodiments, R¹ is 3-membered heterocyclyl substituted with at least one —OR^{G5}. In some embodiments, R¹ is 4-membered heterocyclyl substituted with at least one —OR^{G5}.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —SR^{G5}.

In some embodiments, R¹ is 3-membered heterocyclyl substituted with at least one —SR^{G5}. In some embodiments, R¹ is 4-membered heterocyclyl substituted with at least one —SR^{G5}.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —N(R^{G5})₂.

In some embodiments, R¹ is 3-membered heterocyclyl substituted with at least one —N(R^{G5})₂. In some embodiments, R¹ is 4-membered heterocyclyl substituted with at least one —N(R^{G5})₂.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂.

In some embodiments, R¹ and G₂, together with the atoms to which they are attached, are joined to form an unsubstituted 5-membered heteroaryl ring.

In some embodiments, R¹ and G₂, together with the atoms to which they are attached, are joined to form a 5-membered heteroaryl ring substituted with 1 R^{G7} selected from the group consisting of halo, C₁₋₆alkyl, C₁₋₆haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 2 R^{G7} selected from the group consisting of halo, C₁₋₆ alkyl, C₁₋₆ haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 3 R^{G7} selected from the group consisting of halo, C₁₋₆ alkyl, C₁₋₆ haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆ alkyl, C₁₋₆ haloalkyl, —OR^{G5}, —SR^{G5}, and —N(R^{G5})₂, provided at least one substituent is halo.

In some embodiments, R¹ and G₂, together with the atoms to which they are attached, are joined to form a 5-membered heteroaryl ring substituted with at least one of F, Cl, Br, or I. In some embodiments, R¹ and G₂, together with the atoms to which they are attached, are joined to form a 5-membered heteroaryl ring substituted with at least one of F, Cl, or Br. In some embodiments, R¹ and G₂, together with the atoms to which they are attached, are joined to form a 5-membered heteroaryl ring substituted with at least one of F or Cl.

In some embodiments, R¹ and G₂, together with the atoms to which they are attached, are joined to form a 5-membered heteroaryl ring substituted with at least one F. In some embodiments, R¹ and G₂, together with the atoms to which they are attached, are joined to form a 5-membered heteroaryl ring substituted with at least one Cl. In some embodiments, R¹ and G₂, together with the atoms to which they are attached, are joined to form a 5-membered heteroaryl ring substituted with at least one Br. In some embodiments, R¹ and G₂, together with the atoms to which they are attached, are joined to form a 5-membered heteroaryl ring substituted with at least one I.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆ alkyl, C₁₋₆ haloalkyl, —OR^{G5}, —SR^{G5}, and —N(R^{G5})₂, provided at least one substituent is C₁₋₆ alkyl.

In some embodiments, R¹ and G₂, together with the atoms to which they are attached, are joined to form a 5-membered heteroaryl ring substituted with at least one methyl. In some embodiments, R¹ and G₂, together with the atoms to which they are attached, are joined to form a 5-membered heteroaryl ring substituted with at least one ethyl. In some embodiments, R¹ and G₂, together with the atoms to which they are attached, are joined to form a 5-membered heteroaryl ring substituted with at least one propyl. In some embodiments, R¹ and G₂, together with the atoms to which they are attached, are joined to form a 5-membered heteroaryl ring substituted with at least one butyl. In some embodiments, R¹ and G₂, together with the atoms to which they are attached, are joined to form a 5-membered heteroaryl ring substituted with at least one pentyl. In some embodiments, R¹ and G₂, together with the atoms to which they are attached, are joined to form a 5-membered heteroaryl ring substituted with at least one hexyl. In some embodiments, R¹ and G₂, together with the atoms to which they are attached, are joined to form a 5-membered heteroaryl ring substituted with at least one isopropyl. In some embodiments, R¹ and G₂, together with the atoms to which they are attached, are joined to form a 5-membered heteroaryl ring substituted with at least one isobutyl. In some embodiments, R¹ and G₂, together with the atoms to which they are attached, are joined to form a 5-membered heteroaryl ring substituted with at least one isopentyl. In some embodiments, R¹ and G₂, together with the atoms to which they are attached, are joined to form a 5-membered heteroaryl ring substituted with at least one isohexyl. In some embodiments, R¹ and G₂, together with the atoms to which they are attached, are joined to form a 5-membered heteroaryl ring substituted with at least one secbutyl. In some embodiments, R¹ and G₂, together with the atoms to which they are attached, are joined to form a 5-membered heteroaryl ring substituted with at least one secpentyl. In some embodiments, R¹ and G₂, together with the atoms to which they are attached, are joined to form a 5-membered heteroaryl ring substituted with at least one sechexyl. In some embodiments, R¹ and G₂, together with the atoms to which they are attached, are joined to form a 5-membered heteroaryl ring substituted with at least one tertbutyl.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is C₁₋₆ haloalkyl.

In some embodiments, R¹ and G₂, together with the atoms to which they are attached, are joined to form a 5-membered heteroaryl ring substituted with at least one halomethyl. In some embodiments, R¹ and G₂, together with the atoms to which they are attached, are joined to form a 5-membered heteroaryl ring substituted with at least one haloethyl. In some embodiments, R¹ and G₂, together with the atoms to which they are attached, are joined to form a 5-membered heteroaryl ring substituted with at least one halopropyl. In some embodiments, R¹ and G₂, together with the atoms to which they are attached, are joined to form a 5-membered heteroaryl ring substituted with at least one halobutyl. In some embodiments, R¹ and G₂, together with the atoms to which they are attached, are joined to form a 5-membered heteroaryl ring substituted with at least one halopentyl. In some embodiments, R¹ and G₂, together with the atoms to which they are attached, are joined to form a 5-membered heteroaryl ring substituted with at least one halohexyl.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is —OR^{G5}.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is —SR^{G5}.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆ alkyl, C₁₋₆ haloalkyl, —OR^{G5}, —SR^{G5}, and —N(R^{G5})₂, provided at least one substituent is —N(R^{G5})₂.

As generally defined herein, each instance of R^{G7} is independently halo, C₁₋₆ alkyl, C₁₋₆ haloalkyl, —OR^{G5}, —SR^{G5}, or —N(R^{G5})₂.

In some embodiments, R^{G7} is halo.

In some embodiments, R^{G7} is F, Cl, Br, or I. In some embodiments, R^{G7} is F, Cl, or Br. In some embodiments, R^{G7} is F or Cl.

In some embodiments, R^{G7} is F. In some embodiments, R^{G7} is Cl. In some embodiments, R^{G7} is Br. In some embodiments, R^{G7} is I.

In some embodiments, R^{G7} is C₁₋₆ alkyl.

In some embodiments, R^{G7} is methyl. In some embodiments, R^{G7} is ethyl. In some embodiments, R^{G7} is propyl. In some embodiments, R^{G7} is butyl. In some embodiments, R^{G7} is pentyl. In some embodiments, R^{G7} is hexyl. In some embodiments, R^{G7} is isopropyl. In some embodiments, R^{G7} is isobutyl. In some embodiments, R^{G7} is isopentyl. In some embodiments, R^{G7} is isohexyl. In some embodiments, R^{G7} is secbutyl. In some embodiments, R^{G7} is secpentyl. In some embodiments, R^{G7} is sechexyl. In some embodiments, R^{G7} is tertbutyl.

In some embodiments, R^{G7} is C₁₋₆ haloalkyl.

In some embodiments, R^{G7} is halomethyl. In some embodiments, R^{G7} is haloethyl. In some embodiments, R^{G7} is halopropyl. In some embodiments, R^{G7} is halobutyl. In some embodiments, R^{G7} is halopentyl. In some embodiments, R^{G7} is halohexyl.

In some embodiments, R^{G7} is —OR^{G5}.

In some embodiments, R^{G7} is —SR^{G5}.

In some embodiments, R^{G7} is —N(R^{G5})₂.

As generally defined herein, R^{G1}, R^{G2}, R^{G3}, and R^{G4} are each independently selected from the group consisting of hydrogen, halo, C₁₋₆ alkyl, C₁₋₆ haloalkyl, and —OR^{G6}.

In some embodiments, R^{G1} is selected from the group consisting of hydrogen, halo, C₁₋₆ alkyl, C₁₋₆ haloalkyl, and —OR^{G6}.

In some embodiments, R^{G1} is hydrogen.

In some embodiments, R^{G1} is halo.

In some embodiments, R^{G1} is F, Cl, Br, or I. In some embodiments, R^{G1} is F, Cl, or Br. In some embodiments, R^{G1} is F or Cl.

In some embodiments, R^{G1} is F. In some embodiments, R^{G1} is Cl. In some embodiments, R^{G1} is Br. In some embodiments, R^{G1} is I.

In some embodiments, R^{G1} is C₁₋₆ alkyl.

In some embodiments, R^{G1} is methyl. In some embodiments, R^{G1} is ethyl. In some embodiments, R^{G1} is propyl. In some embodiments, R^{G1} is butyl. In some embodiments, R^{G1} is pentyl. In some embodiments, R^{G1} is hexyl. In some embodiments, R^{G1} is isopropyl. In some embodiments, R^{G1} is isobutyl. In some embodiments, R^{G1} is isopentyl. In some embodiments, R^{G1} is isohexyl. In some embodiments, R^{G1} is secbutyl. In some embodiments, R^{G1} is secpentyl. In some embodiments, R^{G1} is sechexyl. In some embodiments, R^{G1} is tertbutyl.

In some embodiments, R^{G1} is C₁₋₆haloalkyl.

In some embodiments, R^{G1} is halomethyl. In some embodiments, R^{G1} is haloethyl. In some embodiments, R^{G1} is halopropyl. In some embodiments, R^{G1} is halobutyl. In some embodiments, R^{G1} is halopentyl. In some embodiments, R^{G1} is halohexyl.

In some embodiments, R^{G1} is —OR^{G6}.

In some embodiments, R^{G2} is selected from the group consisting of hydrogen, halo, C₁₋₆ alkyl, C₁₋₆ haloalkyl, and —OR^{G6}.

In some embodiments, R^{G2} is hydrogen.

In some embodiments, R^{G2} is halo.

In some embodiments, R^{G2} is F, Cl, Br, or I. In some embodiments, R^{G2} is F, Cl, or Br. In some embodiments, R^{G2} is F or Cl.

In some embodiments, R^{G2} is F. In some embodiments, R^{G2} is Cl. In some embodiments, R^{G2} is Br. In some embodiments, R^{G2} is I.

In some embodiments, R^{G2} is C₁₋₆ alkyl.

In some embodiments, R^{G2} is methyl. In some embodiments, R^{G2} is ethyl. In some embodiments, R^{G2} is propyl. In some embodiments, R^{G2} is butyl. In some embodiments, R^{G2} is pentyl. In some embodiments, R^{G2} is hexyl. In some embodiments, R^{G2} is isopropyl. In some embodiments, R^{G2} is isobutyl. In some embodiments, R^{G2} is isopentyl. In some embodiments, R^{G2} is isohexyl. In some embodiments, R^{G2} is secbutyl. In some embodiments, R^{G2} is secpentyl. In some embodiments, R^{G2} is sechexyl. In some embodiments, R^{G2} is tertbutyl.

In some embodiments, R^{G2} is C₁₋₆haloalkyl.

In some embodiments, R^{G2} is halomethyl. In some embodiments, R^{G2} is haloethyl. In some embodiments, R^{G2} is halopropyl. In some embodiments, R^{G2} is halobutyl. In some embodiments, R^{G2} is halopentyl. In some embodiments, R^{G2} is halohexyl.

In some embodiments, R^{G2} is —OR^{G6}.

In some embodiments, R^{G3} is selected from the group consisting of hydrogen, halo, C₁₋₆ alkyl, C₁₋₆ haloalkyl, and —OR^{G6}.

In some embodiments, R^{G3} is hydrogen.

In some embodiments, R^{G3} is halo.

In some embodiments, R^{G3} is F, Cl, Br, or I. In some embodiments, R^{G3} is F, Cl, or Br. In some embodiments, R^{G3} is F or Cl.

In some embodiments, R^{G3} is F. In some embodiments, R^{G3} is Cl. In some embodiments, R^{G3} is Br. In some embodiments, R^{G3} is I.

In some embodiments, R^{G3} is C₁₋₆ alkyl.

In some embodiments, R^{G3} is methyl. In some embodiments, R^{G3} is ethyl. In some embodiments, R^{G3} is propyl. In some embodiments, R^{G3} is butyl. In some embodiments, R^{G3} is pentyl. In some embodiments, R^{G3} is hexyl. In some embodiments, R^{G3} is isopropyl. In some embodiments, R^{G3} is isobutyl. In some embodiments, R^{G3} is isopentyl. In some embodiments, R^{G3} is isohexyl. In some embodiments, R^{G3} is secbutyl. In some embodiments, R^{G3} is secpentyl. In some embodiments, R^{G3} is sechexyl. In some embodiments, R^{G3} is tertbutyl.

In some embodiments, R^{G3} is C₁₋₆ haloalkyl.

In some embodiments, R^{G3} is halomethyl. In some embodiments, R^{G3} is haloethyl. In some embodiments, R^{G3} is halopropyl. In some embodiments, R^{G3} is halobutyl. In some embodiments, R^{G3} is halopentyl. In some embodiments, R^{G3} is halohexyl.

In some embodiments, R^{G3} is —OR^{G6}.

In some embodiments, R^{G4} is selected from the group consisting of hydrogen, halo, C₁₋₆ alkyl, C₁₋₆ haloalkyl, and —OR^{G6}.

In some embodiments, R^{G4} is hydrogen.

In some embodiments, R^{G4} is halo.

In some embodiments, R^{G4} is F, Cl, Br, or I. In some embodiments, R^{G4} is F, Cl, or Br. In some embodiments, R^{G4} is F or Cl.

In some embodiments, R^{G4} is F. In some embodiments, R^{G4} is Cl. In some embodiments, R^{G4} is Br. In some embodiments, R^{G4} is I.

In some embodiments, R^{G4} is C₁₋₆ alkyl.

In some embodiments, R^{G4} is methyl. In some embodiments, R^{G4} is ethyl. In some embodiments, R^{G4} is propyl. In some embodiments, R^{G4} is butyl. In some embodiments, R^{G4} is pentyl. In some embodiments, R^{G4} is hexyl. In some embodiments, R^{G4} is isopropyl. In some embodiments, R^{G4} is isobutyl. In some embodiments, R^{G4} is isopentyl. In some embodiments, R^{G4} is isohexyl. In some embodiments, R^{G4} is secbutyl. In some embodiments, R^{G4} is secpentyl. In some embodiments, R^{G4} is sechexyl. In some embodiments, R^{G4} is tertbutyl.

In some embodiments, R^{G4} is C₁₋₆ haloalkyl.

In some embodiments, R^{G4} is halomethyl. In some embodiments, R^{G4} is haloethyl. In some embodiments, R^{G4} is halopropyl. In some embodiments, R^{G4} is halobutyl. In some embodiments, R^{G4} is halopentyl. In some embodiments, R^{G4} is halohexyl.

In some embodiments, R^{G4} is —OR^{G6}.

As generally defined herein, R^{G5} and R^{G6} are each independently hydrogen, C₁₋₆ alkyl, or C₁₋₆ haloalkyl.

In some embodiments, R^{G5} is hydrogen.

In some embodiments, R^{G5} is C₁₋₆ alkyl.

In some embodiments, R^{G5} is methyl. In some embodiments, R^{G5} is ethyl. In some embodiments, R^{G5} is propyl. In some embodiments, R^{G5} is butyl. In some embodiments, R^{G5} is pentyl. In some embodiments, R^{G5} is hexyl. In some embodiments, R^{G5} is isopropyl. In some embodiments, R^{G5} is isobutyl. In some embodiments, R^{G5} is isopentyl. In some embodiments, R^{G5} is isohexyl. In some embodiments, R^{G5} is secbutyl. In some embodiments, R^{G5} is secpentyl. In some embodiments, R^{G5} is sechexyl. In some embodiments, R^{G5} is tertbutyl.

In some embodiments, R^{G5} is C₁₋₆ haloalkyl.

In some embodiments, R^{G5} is halomethyl. In some embodiments, R^{G5} is haloethyl. In some embodiments, R^{G5} is halopropyl. In some embodiments, R^{G5} is halobutyl. In some embodiments, R^{G5} is halopentyl. In some embodiments, R^{G5} is halohexyl.

In some embodiments, R^{G6} is hydrogen.

In some embodiments, R^{G6} is C₁₋₆ alkyl.

In some embodiments, R^{G6} is methyl. In some embodiments, R^{G6} is ethyl. In some embodiments, R^{G6} is propyl. In some embodiments, R^{G6} is butyl. In some embodiments, R^{G6} is pentyl. In some embodiments, R^{G6} is hexyl. In some embodiments, R^{G6} is isopropyl. In some embodiments, R^{G6} is isobutyl. In some embodiments, R^{G6} is isopentyl. In some embodiments, R^{G6} is isohexyl. In some embodiments, R^{G6} is secbutyl. In some embodiments, R^{G6} is secpentyl. In some embodiments, R^{G6} is sechexyl. In some embodiments, R^{G6} is tertbutyl.

In some embodiments, R^{G6} is C₁₋₆ haloalkyl.

In some embodiments, R^{G6} is halomethyl. In some embodiments, R^{G6} is haloethyl. In some embodiments, R^{G6} is halopropyl. In some embodiments, R^{G6} is halobutyl. In some embodiments, R^{G6} is halopentyl. In some embodiments, R^{G6} is halohexyl.

In some embodiments, Ring A of formula:

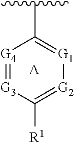

is a group of formula:

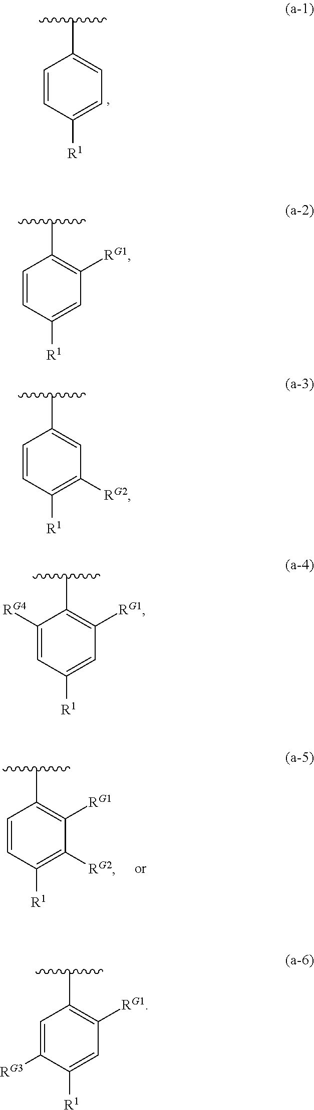

In some embodiments, Ring A is of formula (a-2), (a-3), (a-4), (a-5), or (a-6), wherein R^{G1}, R^{G2}, R^{G3}, and R^{G4} are each independently selected from the group consisting of halo, C₁₋₆ alkyl, C₁₋₆ haloalkyl, and —OR^{G6}.

In some embodiments, Ring A is of formula (a-2), (a-4), (a-5), or (a-6), wherein R^{G1} is —OR^{G6}.

In some embodiments, Ring A is of formula:

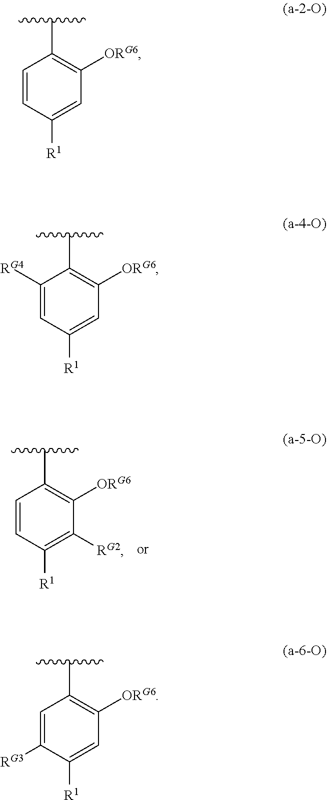

In some embodiments, Ring A is of formula (a-2), (a-4), (a-5), or (a-6), wherein R^{G1} is fluoro.

In some embodiments, Ring A is of formula:

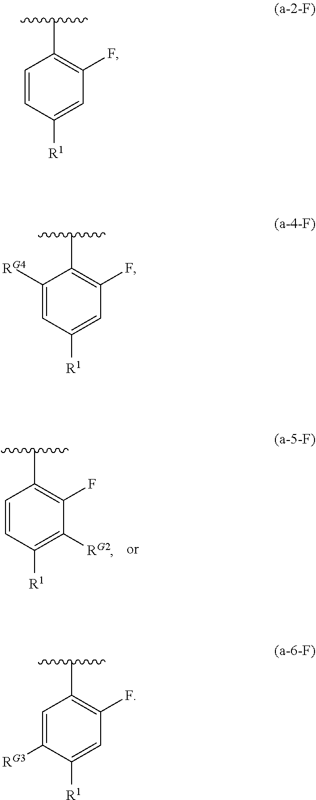

In some embodiments, Ring A of formula:

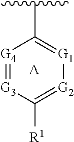

is a group of formula:

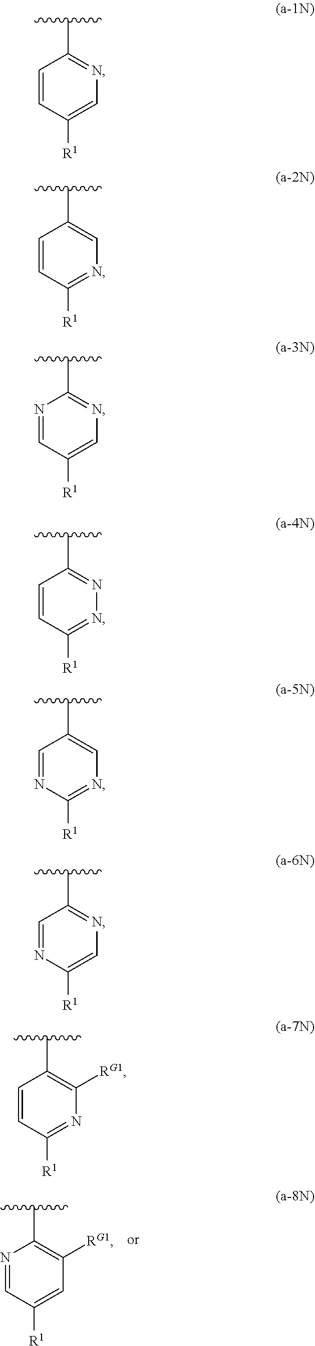

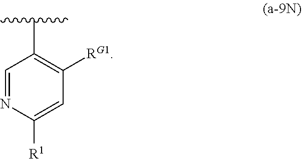

In some embodiments, Ring A is of formula (a-1N), (a-2N), (a-3N), (a-4N), (a-5N), (a-6N), (a-7N), (a-8N), or (a-9N), wherein R^{G1} is halo, C₁₋₆alkyl, C₁₋₆haloalkyl, or —OR^{G6}.

In some embodiments, Ring A is of formula (a-7N), (a-8N), or (a-9N), wherein R^{G1} is —OR^{G6}.

In some embodiments, Ring A is of formula:

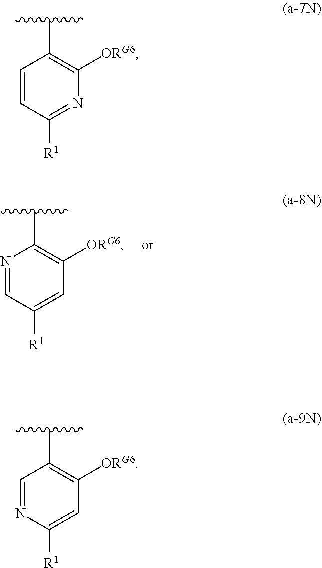

In some embodiments, Ring A is of formula:

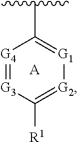

and R¹ and G₂, together with the atoms to which they are attached, are joined to form a 5-membered heteroaryl ring, wherein the Ring A, R¹, and G² provide a group of formula:

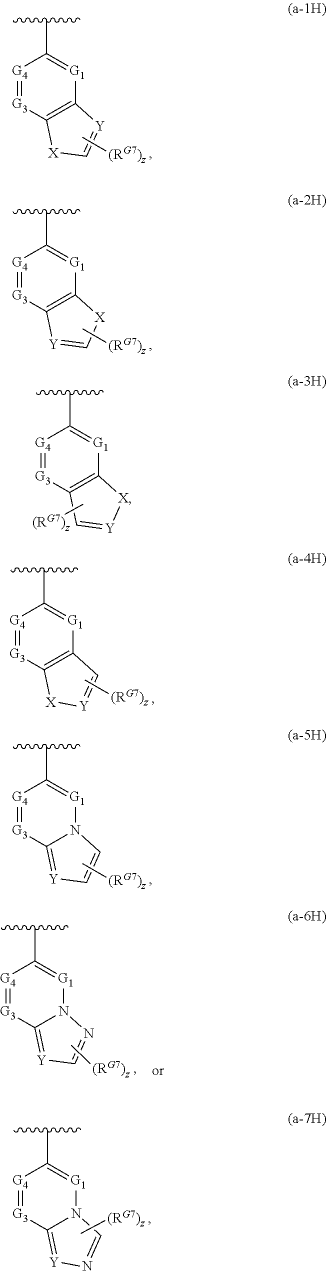

wherein:
   X is O, S, NH, or NR^{G7};
   Y is N, CH, or CR^{G7}; and
   z is 0 or 1;
   provided if R^{G7} is a group attached to a nitrogen (N) atom, then R^{G7} is C₁₋₆ alkyl or C₁₋₆ haloalkyl.

In some embodiments, X is O or S.

In some embodiments, X is O. In some embodiments, X is S.

In some embodiments, X is NH or NR^{G7}.

In some embodiments, X is NH. In some embodiments, X is NR^{G7}.

In some embodiments, Y is N.

In some embodiments, Y is CH or CR^{G7}.

In some embodiments, Y is CH. In some embodiments, Y is CR^{G7}.

In some embodiments, z is 0.

In some embodiments, z is 1.

In some embodiments, if R^{G7} is a group attached to a nitrogen (N) atom, then R^{G7} is C₁₋₆ alkyl.

In some embodiments, if R^{G7} is a group attached to a nitrogen (N) atom, then R^{G7} is C₁₋₆ haloalkyl.

In some embodiments, Ring A, when R¹ and G₂, together with the atoms to which they are attached, are joined to form a 5-membered heteroaryl ring, wherein the Ring A, R¹, and G² provide a group of formula:

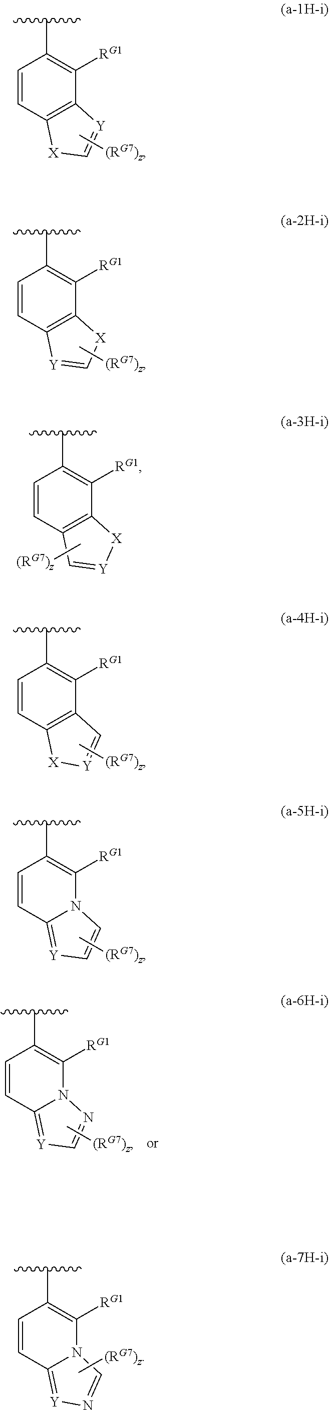

In some embodiments, Ring A is a group of formula:

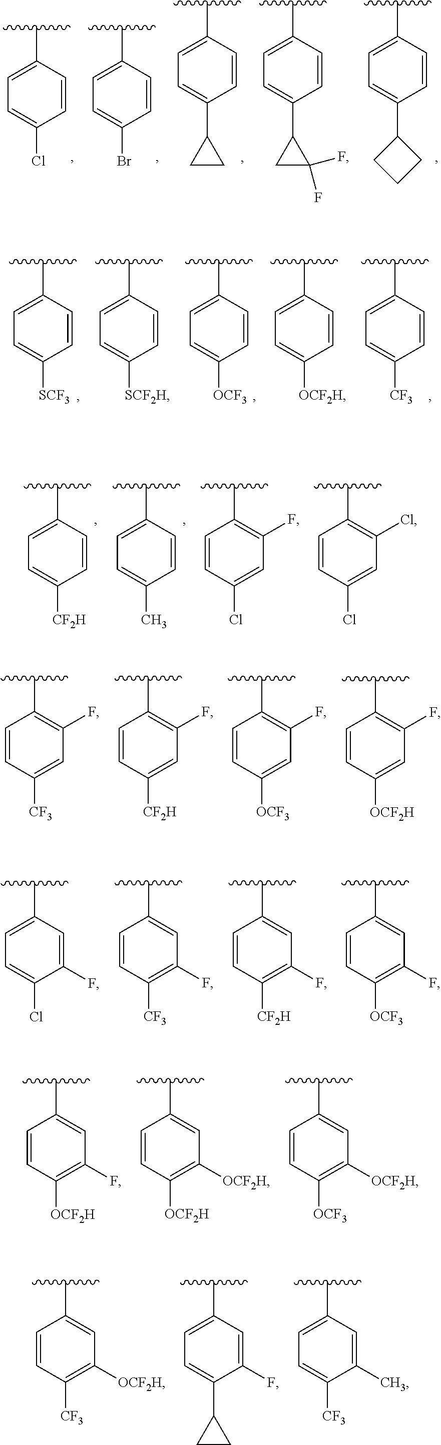

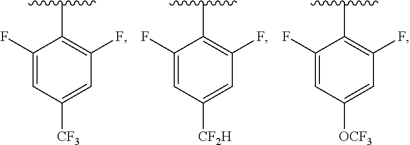

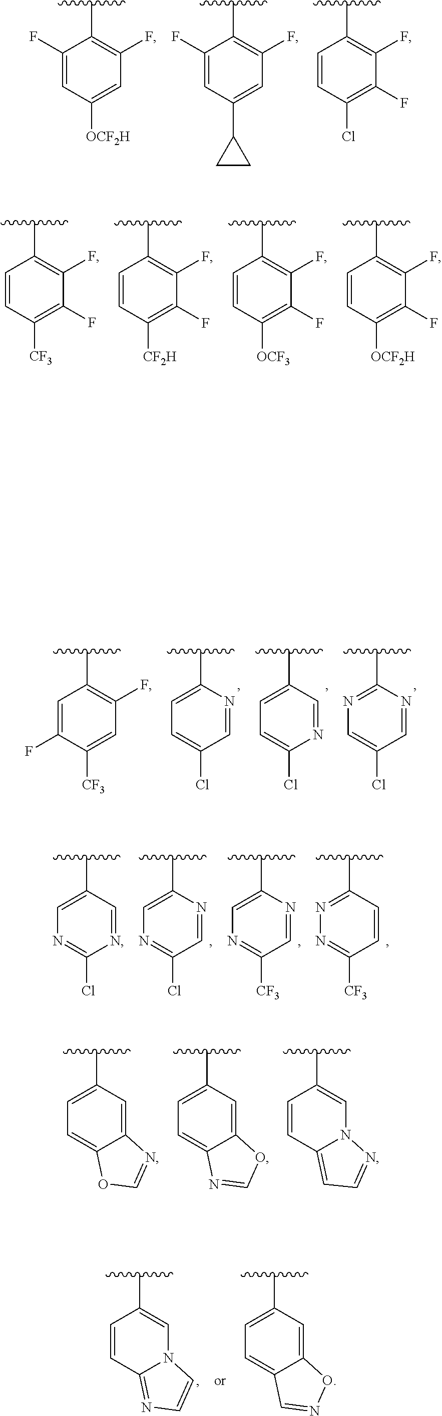

In some embodiments, Ring A is a group of formula:

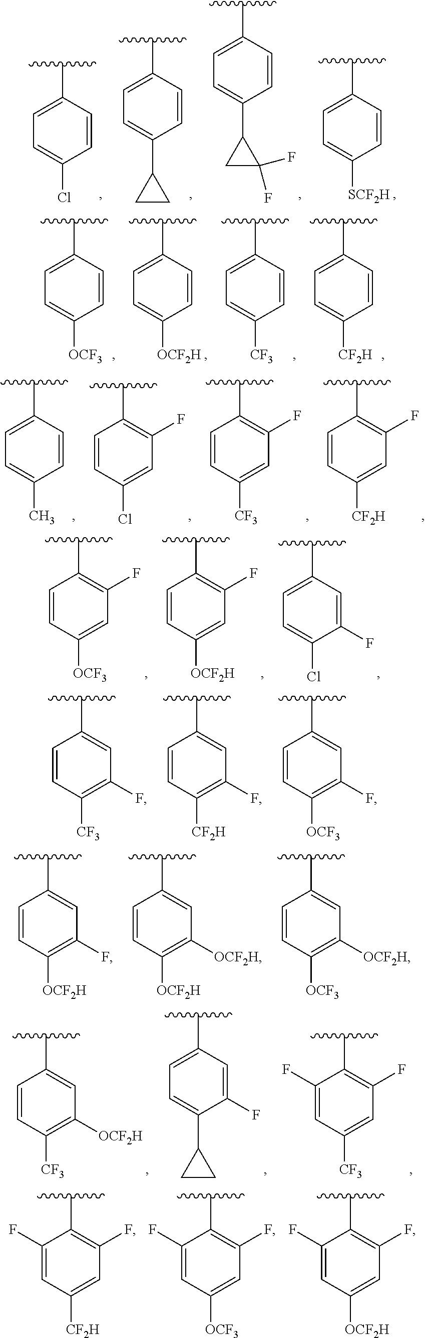

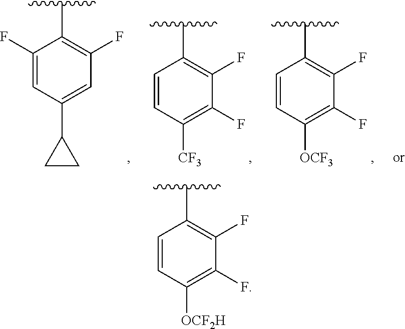

In some embodiments, Ring A is a group of formula:

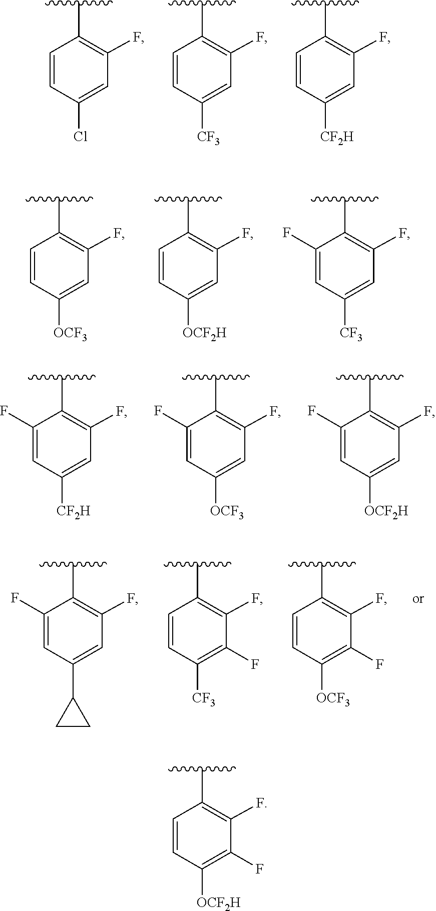

In some embodiments, Ring A is a group of formula:

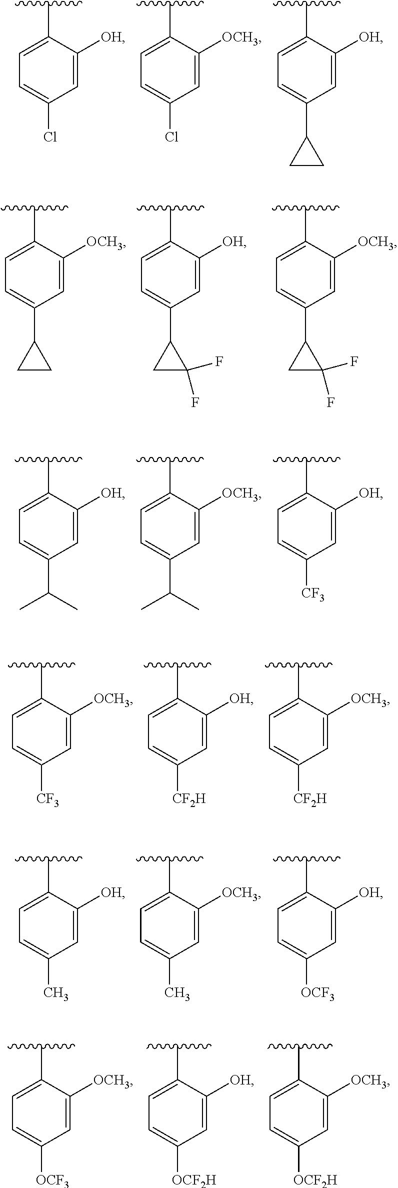

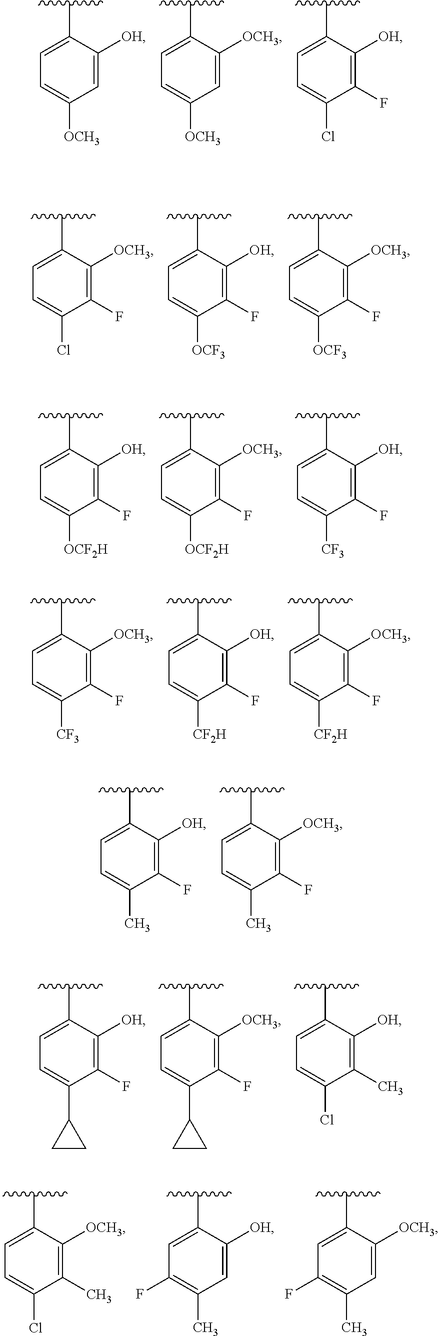

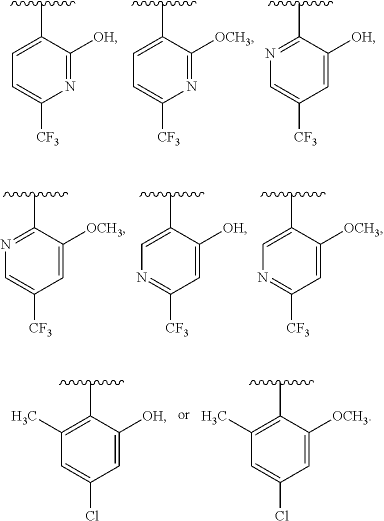

In some embodiments, Ring A is a group of formula:

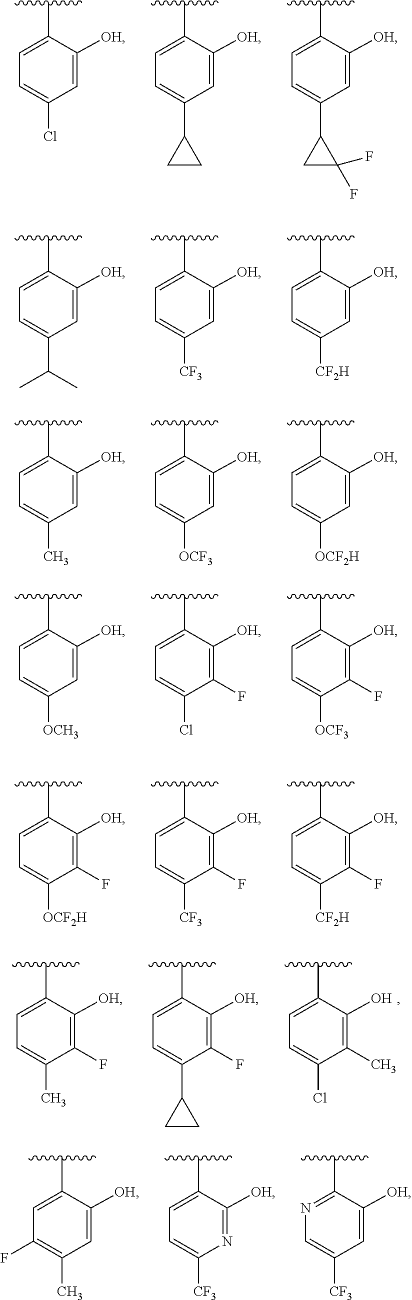

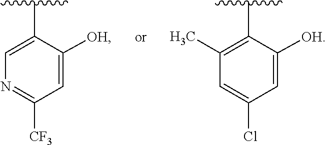

In some embodiments, Ring A is a group of formula:

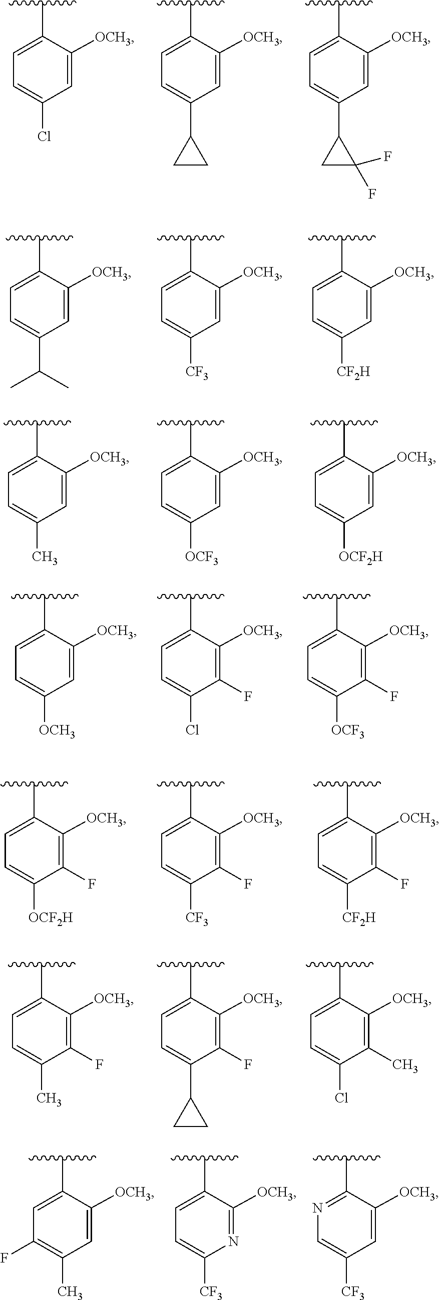

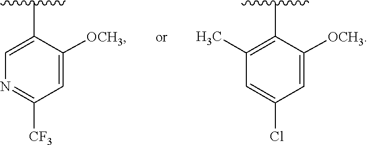 [SMILES: *c1cnc(C(F)(F)F)cc1OC ; *c1c(C)cc(Cl)cc1OC]

In some embodiments, Ring A is a group of formula:

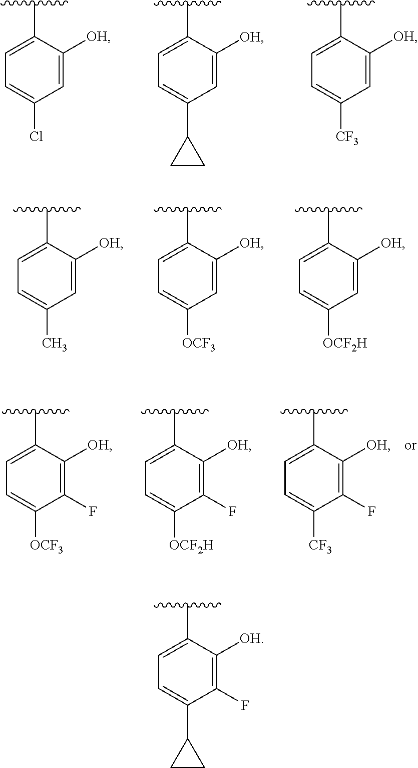

In some embodiments, Ring A is a group of formula:

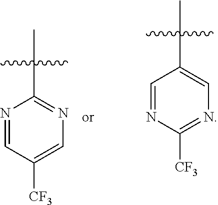 [SMILES: *c1ncc(C(F)(F)F)cn1 ; *c1cnc(C(F)(F)F)nc1]

(b) Ring B, n, p, m, R³, R⁴, R^{2a}, and R^{2b}

As generally described herein, n is 0 or 1.

In some embodiments, n is 0 or 1.

In some embodiments, n is 0. In some embodiments, n is 1.

As generally described herein, p is 1 or 2.

In some embodiments, p is 1. In some embodiments, p is 2.

As generally described herein, m is 0, 1, 2, or 3.

In some embodiments, m is 1, 2, or 3. In some embodiments, m is 0, 1, or 2. In some embodiments, m is 1 or 2. In some embodiments, m is 2 or 3. In some embodiments, m is 1 or 3.

In some embodiments, m is 0. In some embodiments, m is 1. In some embodiments, m is 2. In some embodiments, m is 3.

As generally described herein, each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo.

In some embodiments, R^{2a} is independently hydrogen, halo, C₁₋₆ alkyl, C₁₋₆ haloalkyl, C₃-C₄ carbocyclyl, or 3-4 membered heterocyclyl.

In some embodiments, R^{2a} is independently hydrogen.

In some embodiments, R^{2a} is independently halo.

In some embodiments, R^{2a} is independently F, Cl, Br, or I. In some embodiments, R^{2a} is independently F, Cl, or Br. In some embodiments, R^{2a} is independently F or Cl.

In some embodiments, R^{2a} is independently F. In some embodiments, R^{2a} is independently Cl. In some embodiments, R^{2a} is independently Br. In some embodiments, R^{2a} is independently I.

In some embodiments, R^{2a} is independently C₁₋₆ alkyl.

In some embodiments, R^{2a} is independently methyl. In some embodiments, R^{2a} is independently ethyl. In some embodiments, R^{2a} is independently propyl. In some embodiments, R^{2a} is independently butyl. In some embodiments, R^{2a} is independently pentyl. In some embodiments, R^{2a} is independently hexyl. In some embodiments, R^{2a} is independently isopropyl. In some embodiments, R^{2a} is independently isobutyl. In some embodiments, R^{2a} is independently isopentyl. In some embodiments, R^{2a} is independently isohexyl. In some embodiments, R^{2a} is independently secbutyl. In some embodiments, R^{2a} is independently secpentyl. In some embodiments, R^{2a} is independently sechexyl. In some embodiments, R^{2a} is independently tertbutyl.

In some embodiments, R^{2a} is independently C₁₋₆ haloalkyl.

In some embodiments, R^{2a} is independently halomethyl. In some embodiments, R^{2a} is independently haloethyl. In some embodiments, R^{2a} is independently halopropyl. In some embodiments, R^{2a} is independently halobutyl. In some embodiments, R^{2a} is independently halopentyl. In some embodiments, R^{2a} is independently halohexyl.

In some embodiments, R^{2a} is independently C₃-C₄ carbocyclyl.

In some embodiments, R^{2a} is independently C₃ carbocyclyl. In some embodiments, R^{2a} is independently C₄ carbocyclyl.

In some embodiments, R^{2a} is independently 3-4 membered heterocyclyl.

In some embodiments, R^{2a} is independently 3-membered heterocyclyl. In some embodiments, R^{2a} is independently 4-membered heterocyclyl.

In some embodiments, each instance of R^{2b} is independently hydrogen, halo, C₁₋₆ alkyl, C₁₋₆ haloalkyl, C₃-C₄ carbocyclyl, or 3-4 membered heterocyclyl.

In some embodiments, R^{2b} is independently hydrogen.

In some embodiments, R^{2b} is independently halo.

In some embodiments, R^{2b} is independently F, Cl, Br, or I. In some embodiments, R^{2b} is independently F, Cl, or Br. In some embodiments, R^{2b} is independently F or Cl.

In some embodiments, R^{2b} is independently F. In some embodiments, R^{2b} is independently Cl. In some embodiments, R^{2b} is independently Br. In some embodiments, R^{2b} is independently I.

In some embodiments, R^{2b} is independently C₁₋₆ alkyl.

In some embodiments, R^{2b} is independently methyl. In some embodiments, R^{2b} is independently ethyl. In some embodiments, R^{2b} is independently propyl. In some embodiments, R^{2b} is independently butyl. In some embodiments, R^{2b} is independently pentyl. In some embodiments, R^{2b} is independently hexyl. In some embodiments, R^{2b} is independently isopropyl. In some embodiments, R^{2b} is independently isobutyl. In some embodiments, R^{2b} is independently isopentyl. In some embodiments, R^{2b} is independently isohexyl. In some embodiments, R^{2b} is independently secbutyl. In some embodiments, R^{2b} is independently secpentyl. In some embodiments, R^{2b} is independently sechexyl. In some embodiments, R^{2b} is independently tertbutyl.

In some embodiments, R^{2b} is independently C₁₋₆ haloalkyl.

In some embodiments, R^{2b} is independently halomethyl. In some embodiments, R^{2b} is independently haloethyl. In some embodiments, R^{2b} is independently halopropyl. In some embodiments, R^{2b} is independently halobutyl. In some embodiments, R^{2b} is independently halopentyl. In some embodiments, R^{2b} is independently halohexyl.

In some embodiments, R^{2b} is independently C₃-C₄ carbocyclyl.

In some embodiments, R^{2b} is independently C₃ carbocyclyl. In some embodiments, R^{2b} is independently C₄ carbocyclyl.

In some embodiments, R^{2b} is independently 3-4 membered heterocyclyl.

In some embodiments, R^{2b} is independently 3-membered heterocyclyl. In some embodiments, R^{2b} is independently 4-membered heterocyclyl.

In some embodiments, R^{2a} and R^{2b} are the same. In some embodiments, R^{2a} and R^{2b} are different.

In some embodiments, R^{2a} and R^{2b} are joined to form a C₃ carbocyclyl independently substituted with 0, 1, 2, or 3 halo.

As generally described herein, each instance of R³ is independently halo, C₁₋₆ alkyl or C₁₋₆ haloalkyl, or two R³ groups are joined to form a C₁₋₃ alkylene bridging group or C₁₋₃ haloalkylene bridging group.

In some embodiments, R³ is halo.

In some embodiments, R³ is F, Cl, Br, or I. In some embodiments, R³ is F, Cl, or Br. In some embodiments, R³ is F or Cl.

In some embodiments, R³ is F. In some embodiments, R³ is Cl. In some embodiments, R³ is Br. In some embodiments, R³ is I.

In some embodiments, R³ is C₁₋₆alkyl.

In some embodiments, R³ is methyl. In some embodiments, R³ is ethyl. In some embodiments, R³ is propyl. In some embodiments, R³ is butyl. In some embodiments, R³ is pentyl. In some embodiments, R³ is hexyl. In some embodiments, R³ is isopropyl. In some embodiments, R³ is isobutyl. In some embodiments, R³ is isopentyl. In some embodiments, R³ is isohexyl. In some embodiments, R³ is secbutyl. In some embodiments, R³ is secpentyl. In some embodiments, R³ is sechexyl. In some embodiments, R³ is tertbutyl.

In some embodiments, R³ is C₁₋₆haloalkyl.

In some embodiments, R³ is halomethyl. In some embodiments, R³ is haloethyl. In some embodiments, R³ is halopropyl. In some embodiments, R³ is halobutyl. In some embodiments, R³ is halopentyl. In some embodiments, R³ is halohexyl.

As generally described herein, two R³ groups are joined to form a C₁₋₃ alkylene bridging group or a C₁₋₃ haloalkylene bridging group.

In some embodiments, two R³ groups joined to form a bridging group are defined as L.

In some embodiments, two R³ groups are joined to form a C₁₋₃ alkylene bridging group.

In some embodiments, two R³ groups are joined to form a methylene bridging group. In some embodiments, two R³ groups are joined to form an ethylene bridging group. In some embodiments, two R³ groups are joined to form a propylene bridging group.

In some embodiments, two R³ groups are joined to form a C₁₋₃ haloalkylene bridging group.

In some embodiments, two R³ groups are joined to form a halomethylene bridging group. In some embodiments, two R³ groups are joined to form a haloethylene bridging group. In some embodiments, two R³ groups are joined to form a halopropylene bridging group.

As generally described herein, R⁴ is hydrogen, C₁₋₃ alkyl, C₃-C₄ carbocyclyl, or C₃-C₄ carbocyclyl-C₁₋₃ alkyl-, wherein the alkyl and carbocyclyl are each independently substituted with 0, 1, 2, 3, 4, 5, or 6 halo, and wherein the carbocyclyl is further independently substituted with 0, 1, or 2 C₁₋₃ alkyl or C₁₋₃ haloalkyl.

In some embodiments, R⁴ is hydrogen. In other embodiments, R⁴ is not hydrogen.

In some embodiments, R⁴ is C₁₋₃ alkyl.

In some embodiments, R⁴ is methyl. In some embodiments, R⁴ is ethyl. In some embodiments, R⁴ is propyl. In some embodiments, R⁴ is isopropyl.

In some embodiments, R⁴ is C₁₋₃ alkyl substituted with 0, 1, 2, 3, 4, 5, or 6 halo.

In some embodiments, R⁴ is methyl substituted with 0, 1, 2, or 3 halo. In some embodiments, R⁴ is ethyl substituted with 0, 1, 2, 3, 4, 5, or 6 halo. In some embodiments, R⁴ is propyl substituted with 0, 1, 2, 3, 4, 5, or 6 halo. In some embodiments, R⁴ is isopropyl substituted with 0, 1, 2, 3, 4, 5, or 6 halo.

In some embodiments, R⁴ is C₁₋₃ alkyl substituted with 1 halo.

In some embodiments, R⁴ is methyl substituted with 1 halo. In some embodiments, R⁴ is ethyl substituted with 1 halo. In some embodiments, R⁴ is propyl substituted with 1 halo. In some embodiments, R⁴ is isopropyl substituted with 1 halo.

In some embodiments, R⁴ is C₁₋₃ alkyl independently substituted with 2 halo.

In some embodiments, R⁴ is methyl independently substituted with 2 halo. In some embodiments, R⁴ is ethyl independently substituted with 2 halo. In some embodiments, R⁴ is propyl independently substituted with 2 halo. In some embodiments, R⁴ is isopropyl independently substituted with 2 halo.

In some embodiments, R⁴ is C₁₋₃ alkyl independently substituted with 3 halo.

In some embodiments, R⁴ is methyl independently substituted with 3 halo. In some embodiments, R⁴ is ethyl independently substituted with 3 halo. In some embodiments, R⁴ is propyl independently substituted with 3 halo. In some embodiments, R⁴ is isopropyl independently substituted with 3 halo.

In some embodiments, R⁴ is C₁₋₃ alkyl independently substituted with 4 halo.

In some embodiments, R⁴ is ethyl independently substituted with 4 halo. In some embodiments, R⁴ is independently propyl substituted with 4 halo. In some embodiments, R⁴ is isopropyl independently substituted with 4 halo.

In some embodiments, R⁴ is C₁₋₃ alkyl substituted with 5 halo.

In some embodiments, R⁴ is ethyl independently substituted with 5 halo. In some embodiments, R⁴ is propyl independently substituted with 5 halo. In some embodiments, R⁴ is isopropyl independently substituted with 5 halo.

In some embodiments, R⁴ is C₁₋₃ alkyl substituted with 6 halo.

In some embodiments, R⁴ is ethyl independently substituted with 6 halo. In some embodiments, R⁴ is independently propyl substituted with 6 halo. In some embodiments, R⁴ is independently isopropyl substituted with 6 halo.

In some embodiments, R⁴ is C₁₋₃ alkyl substituted with at least one of F, Cl, Br, or I. In some embodiments, R⁴ is C₁₋₃ alkyl substituted with at least one of F, Cl, or Br. In some embodiments, R⁴ is C₁₋₃ alkyl substituted with at least one of F or Cl.

In some embodiments, R⁴ is methyl substituted with at least one of F, Cl, Br, or I. In some embodiments, R⁴ is methyl substituted with at least one of F, Cl, or Br. In some embodiments, R⁴ is methyl substituted with at least one of F or Cl.

In some embodiments, R⁴ is ethyl substituted with at least one of F, Cl, Br, or I. In some embodiments, R⁴ is ethyl substituted with at least one of F, Cl, or Br. In some embodiments, R⁴ is ethyl substituted with at least one of F or Cl.

In some embodiments, R⁴ is propyl substituted with at least one of F, Cl, Br, or I. In some embodiments, R⁴ is propyl substituted with at least one of F, Cl, or Br. In some embodiments, R⁴ is propyl substituted with at least one of F or Cl.

In some embodiments, R⁴ is isopropyl substituted with at least one of F, Cl, Br, or I. In some embodiments, R⁴ is isopropyl substituted with at least one of F, Cl, or Br. In some embodiments, R⁴ is isopropyl substituted with at least one of F or Cl.

In some embodiments, R⁴ is C₁₋₃ alkyl substituted with at least one F. In some embodiments, R⁴ is C₁₋₃ alkyl substituted with at least one Cl. In some embodiments, R⁴ is C₁₋₃ alkyl substituted with at least one Br. In some embodiments, R⁴ is C₁₋₃ alkyl substituted with at least one I.

In some embodiments, R⁴ is methyl substituted with at least one F. In some embodiments, R⁴ is methyl substituted with at least one Cl. In some embodiments, R⁴ is methyl substituted with at least one Br. In some embodiments, R⁴ is methyl substituted with at least one I.

In some embodiments, R⁴ is ethyl substituted with at least one F. In some embodiments, R⁴ is ethyl substituted with at least one Cl. In some embodiments, R⁴ is ethyl substituted with at least one Br. In some embodiments, R⁴ is ethyl substituted with at least one I.

In some embodiments, R⁴ is propyl substituted with at least one F. In some embodiments, R⁴ is propyl substituted with at least one Cl. In some embodiments, R⁴ is propyl substituted with at least one Br. In some embodiments, R⁴ is propyl substituted with at least one I.

In some embodiments, R⁴ is isopropyl substituted with at least one F. In some embodiments, R⁴ is isopropyl substituted with at least one Cl. In some embodiments, R⁴ is isopropyl substituted with at least one Br. In some embodiments, R⁴ is isopropyl substituted with at least one I.

In some embodiments, R⁴ is C₃-C₄ carbocyclyl substituted with 0, 1, 2, 3, 4, 5, or 6 halo and 0, 1, or 2 C₁₋₃ alkyl or C₁₋₃ haloalkyl.

In some embodiments, R⁴ is C₃ carbocyclyl substituted with 0, 1, 2, 3, 4, or 5 halo. In some embodiments, R⁴ is C₄ carbocyclyl substituted with 0, 1, 2, 3, 4, 5, or 6 halo.

In some embodiments, R⁴ is unsubstituted C₃-C₄ carbocyclyl.

In some embodiments, R⁴ is unsubstituted C₃ carbocyclyl. In some embodiments, R⁴ is unsubstituted C₄ carbocyclyl.

In some embodiments, R⁴ is C₃-C₄ carbocyclyl substituted with 1 halo.

In some embodiments, R⁴ is C₃ carbocyclyl substituted with 1 halo. In some embodiments, R⁴ is C₄ carbocyclyl substituted with 1 halo.

In some embodiments, R⁴ is C₃-C₄ carbocyclyl independently substituted with 2 halo.

In some embodiments, R⁴ is C₃ carbocyclyl independently substituted with 2 halo. In some embodiments, R⁴ is C₄ carbocyclyl substituted with 2 halo.

In some embodiments, R⁴ is C₃-C₄ carbocyclyl independently substituted with 3 halo.

In some embodiments, R⁴ is C₃ carbocyclyl independently substituted with 3 halo. In some embodiments, R⁴ is C₄ carbocyclyl substituted with 3 halo.

In some embodiments, R⁴ is C₃-C₄ carbocyclyl independently substituted with 4 halo.

In some embodiments, R⁴ is C₃ carbocyclyl independently substituted with 4 halo. In some embodiments, R⁴ is C₄ carbocyclyl substituted with 4 halo.

In some embodiments, R⁴ is C₃-C₄ carbocyclyl independently substituted with 5 halo.

In some embodiments, R⁴ is C₃ carbocyclyl independently substituted with 5 halo. In some embodiments, R⁴ is C₄ carbocyclyl substituted with 5 halo.

In some embodiments, R⁴ is C₄ carbocyclyl independently substituted with 6 halo.

In some embodiments, R⁴ is C₃-C₄ carbocyclyl substituted with at least one of F, Cl, Br, or I. In some embodiments, R⁴ is C₃-C₄ carbocyclyl substituted with at least one of F, Cl, or Br. In some embodiments, R⁴ is C₃-C₄ carbocyclyl substituted with at least one of F or Cl.

In some embodiments, R⁴ is C₃ carbocyclyl substituted with at least one of F, Cl, Br, or I. In some embodiments, R⁴ is C₃ carbocyclyl substituted with at least one of F, Cl, or Br. In some embodiments, R⁴ is C₃ carbocyclyl substituted with at least one of F or Cl.

In some embodiments, R⁴ is C₄ carbocyclyl substituted with at least one of F, Cl, Br, or I. In some embodiments, R⁴ is C₄ carbocyclyl substituted with at least one of F, Cl, or Br. In some embodiments, R⁴ is C₄ carbocyclyl substituted with at least one of F or Cl.

In some embodiments, R⁴ is C₃-C₄ carbocyclyl substituted with at least one F. In some embodiments, R⁴ is C₃-C₄ carbocyclyl substituted with at least one Cl. In some embodiments, R⁴ is C₃-C₄ carbocyclyl substituted with at least one Br. In some embodiments, R⁴ is C₃-C₄ carbocyclyl substituted with at least one I.

In some embodiments, R⁴ is C₃ carbocyclyl substituted with at least one F. In some embodiments, R⁴ is C₃ carbocyclyl substituted with at least one Cl. In some embodiments, R⁴ is C₃ carbocyclyl substituted with at least one Br. In some embodiments, R⁴ is C₃ carbocyclyl substituted with at least one I.

In some embodiments, R⁴ is C₄ carbocyclyl substituted with at least one F. In some embodiments, R⁴ is C₄ carbocyclyl substituted with at least one Cl. In some embodiments, R⁴ is C₄ carbocyclyl substituted with at least one Br. In some embodiments, R⁴ is C₄ carbocyclyl substituted with at least one I.

In some embodiments, R⁴ is C₃-C₄ carbocyclyl-C₁₋₃ alkyl-, wherein the alkyl and carbocyclyl are each independently substituted with 0, 1, 2, 3, 4, 5, or 6 halo.

For clarity, the number of substituents provided on the alkyl and the carbocyclyl group should satisfy valency requirements of that group. Additionally, wherein both an alkyl and carbocyclyl are in a variable, either the alkyl or carbocyclyl group may each be independently substituted with up to 6 halo, and carbocyclyl up to 0, 1, or 2 C₁₋₃ alkyl or C₁₋₃ haloalkyl.

In some embodiments, R⁴ is C₃ carbocyclyl-C₁₋₃ alkyl, wherein the carbocyclyl is substituted with 0, 1, 2, 3, 4, or 5 halo and 0, 1, or 2 C₁₋₃ alkyl or C₁₋₃ haloalkyl. In some embodiments, R⁴ is C₄ carbocyclyl-C₁₋₃ alkyl, wherein the carbocyclyl is independently substituted with 0, 1, 2, 3, 4, 5, or 6 halo and 0, 1, or 2 C₁₋₃ alkyl or C₁₋₃ haloalkyl. In some embodiments, the C₁₋₃ alkyl is independently substituted with 0, 1, 2, 3, 4, 5, or 6 halo.

In some embodiments, R⁴ is C₃-C₄ carbocyclyl-C₁ alkyl, wherein the carbocyclyl is independently substituted with 0, 1, 2, 3, 4, 5, or 6 halo and 0, 1, or 2 C₁₋₃ alkyl or C₁₋₃ haloalkyl. In some embodiments, R⁴ is C₃-C₄ carbocyclyl-C₂ alkyl, wherein the carbocyclyl is independently substituted with 0, 1, 2, 3, 4, 5, or 6 halo and 0, 1, or 2 C₁₋₃ alkyl or C₁₋₃ haloalkyl. In some embodiments, R⁴ is C₃-C₄ carbocyclyl-C₃ alkyl, wherein the carbocyclyl is independently substituted with 0, 1, 2, 3, 4, 5, or 6 halo and 0, 1, or 2 C₁₋₃ alkyl or C₁₋₃ haloalkyl. In some embodiments, the C₁₋₃alkyl is independently substituted with 0, 1, 2, 3, 4, 5, or 6 halo.

In some embodiments, R⁴ is unsubstituted C₃-C₄ carbocyclyl-C₁₋₃ alkyl.

In some embodiments, R⁴ is unsubstituted C₃ carbocyclyl-C₁₋₃ alkyl. In some embodiments, R⁴ is unsubstituted C₄ carbocyclyl-C₁₋₃ alkyl.

In some embodiments, R⁴ is unsubstituted C₃-C₄ carbocyclyl-C₁ alkyl. In some embodiments, R⁴ is unsubstituted C₃-C₄ carbocyclyl-C₂ alkyl. In some embodiments, R⁴ is unsubstituted C₃-C₄ carbocyclyl-C₃ alkyl.

In some embodiments, R⁴ is C₃-C₄ carbocyclyl-C₁₋₃ alkyl, wherein the carbocyclyl is substituted with 1 halo. In some embodiments, the C₁₋₃alkyl is independently substituted with 0, 1, 2, 3, 4, 5, or 6 halo.

In some embodiments, R⁴ is C₃ carbocyclyl-C₁₋₃ alkyl, wherein the carbocyclyl is substituted with 1 halo. In some embodiments, R⁴ is C₄ carbocyclyl-C₁₋₃ alkyl, wherein the carbocyclyl is substituted with 1 halo. In some embodiments, the C₁₋₃alkyl is independently substituted with 0, 1, 2, 3, 4, 5, or 6 halo.

In some embodiments, R⁴ is C₃-C₄ carbocyclyl-C₁ alkyl, wherein the carbocyclyl is substituted with 1 halo. In some embodiments, R⁴ is C₃-C₄ carbocyclyl-C₂ alkyl, wherein the carbocyclyl is substituted with 1 halo. In some embodiments, R⁴ is C₃-C₄ carbocyclyl-C₃ alkyl, wherein the carbocyclyl is substituted with 1 halo. In some embodiments, the C₁₋₃alkyl is independently substituted with 0, 1, 2, 3, 4, 5, or 6 halo.

In some embodiments, R⁴ is C₃-C₄ carbocyclyl-C₁₋₃ alkyl, wherein the carbocyclyl is independently substituted with 2 halo. In some embodiments, the C₁₋₃alkyl is independently substituted with 0, 1, 2, 3, 4, 5, or 6 halo.

In some embodiments, R⁴ is C₃ carbocyclyl-C₁₋₃ alkyl, wherein the carbocyclyl is independently substituted with 2 halo. In some embodiments, R⁴ is C₄ carbocyclyl-C₁₋₃ alkyl, wherein the carbocyclyl is independently substituted with 2 halo. In some embodiments, the C₁₋₃alkyl is independently substituted with 0, 1, 2, 3, 4, 5, or 6 halo.

In some embodiments, R⁴ is C₃-C₄ carbocyclyl-C₁ alkyl, wherein the carbocyclyl is independently substituted with 2 halo. In some embodiments, R⁴ is C₃-C₄ carbocyclyl-C₂ alkyl, wherein the carbocyclyl is independently substituted with 2 halo. In some embodiments, R⁴ is C₃-C₄ carbocyclyl-C₃ alkyl, wherein the carbocyclyl is independently substituted with 2 halo. In some embodiments, the C₁₋₃alkyl is independently substituted with 0, 1, 2, 3, 4, 5, or 6 halo.

In some embodiments, R⁴ is C₃-C₄ carbocyclyl-C₁₋₃ alkyl, wherein the carbocyclyl is independently substituted with 3 halo. In some embodiments, the C₁₋₃alkyl is independently substituted with 0, 1, 2, 3, 4, 5, or 6 halo.

In some embodiments, R⁴ is C₃ carbocyclyl-C₁₋₃ alkyl, wherein the carbocyclyl is independently substituted with 3 halo. In some embodiments, R⁴ is C₄ carbocyclyl-C₁₋₃ alkyl, wherein the carbocyclyl is independently substituted with 3 halo. In some embodiments, the C₁₋₃alkyl is independently substituted with 0, 1, 2, 3, 4, 5, or 6 halo.

In some embodiments, R⁴ is C₃-C₄ carbocyclyl-C₁ alkyl, wherein the carbocyclyl is independently substituted with 3 halo. In some embodiments, R⁴ is C₃-C₄ carbocyclyl-C₂ alkyl, wherein the carbocyclyl is independently substituted with 3 halo. In some embodiments, R⁴ is C₃-C₄ carbocyclyl-C₃ alkyl, wherein the carbocyclyl is independently substituted with 3 halo. In some embodiments, the C₁₋₃alkyl is independently substituted with 0, 1, 2, 3, 4, 5, or 6 halo.

In some embodiments, R⁴ is C₃-C₄ carbocyclyl-C₁₋₃ alkyl, wherein the carbocyclyl is independently substituted with 4 halo. In some embodiments, the C₁₋₃alkyl is independently substituted with 0, 1, 2, 3, 4, 5, or 6 halo.

In some embodiments, R⁴ is C₃ carbocyclyl-C₁₋₃ alkyl, wherein the carbocyclyl is independently substituted with 4 halo. In some embodiments, R⁴ is C₄ carbocyclyl-C₁₋₃ alkyl, wherein the carbocyclyl is independently substituted with 4 halo. In some embodiments, the C₁₋₃alkyl is independently substituted with 0, 1, 2, 3, 4, 5, or 6 halo.

In some embodiments, R⁴ is C₃-C₄ carbocyclyl-C₁ alkyl, wherein the carbocyclyl is independently substituted with 4 halo. In some embodiments, R⁴ is C₃-C₄ carbocyclyl-C₂ alkyl, wherein the carbocyclyl is independently substituted with 4 halo. In some embodiments, R⁴ is C₃-C₄ carbocyclyl-C₃ alkyl, wherein the carbocyclyl is independently substituted with 4 halo. In some embodiments, the C₁₋₃alkyl is independently substituted with 0, 1, 2, 3, 4, 5, or 6 halo.

In some embodiments, R⁴ is C₃-C₄ carbocyclyl-C₁₋₃ alkyl, wherein the carbocyclyl is independently substituted with 5 halo. In some embodiments, the C₁₋₃alkyl is independently substituted with 0, 1, 2, 3, 4, 5, or 6 halo.

In some embodiments, R⁴ is C₃ carbocyclyl-C₁₋₃ alkyl, wherein the carbocyclyl is independently substituted with 5 halo. In some embodiments, R⁴ is C₄ carbocyclyl-C₁₋₃ alkyl, wherein the carbocyclyl is independently substituted with 5 halo. In some embodiments, the C₁₋₃alkyl is independently substituted with 0, 1, 2, 3, 4, 5, or 6 halo.

In some embodiments, R⁴ is C₃-C₄ carbocyclyl-C₁ alkyl, wherein the alkyl and carbocyclyl are independently substituted with 5 halo. In some embodiments, R⁴ is C₃-C₄ carbocyclyl-C₂ alkyl, wherein the carbocyclyl is independently substituted with 5 halo. In some embodiments, R⁴ is C₃-C₄ carbocyclyl-C₃ alkyl, wherein the carbocyclyl is independently substituted with 5 halo. In some embodiments, the C₁₋₃alkyl is independently substituted with 0, 1, 2, 3, 4, 5, or 6 halo.

In some embodiments, R⁴ is C₄ carbocyclyl-C₁₋₃ alkyl, wherein the carbocyclyl is independently substituted with 6 halo. In some embodiments, the C₁₋₃alkyl is independently substituted with 0, 1, 2, 3, 4, 5, or 6 halo.

In some embodiments, R⁴ is C₄ carbocyclyl-C₁ alkyl, wherein the carbocyclyl is independently substituted with 6 halo. In some embodiments, R⁴ is C₄ carbocyclyl-C₂ alkyl, wherein the carbocyclyl is independently substituted with 6 halo. In some embodiments, R⁴ is C₄ carbocyclyl-C₃ alkyl, wherein the carbocyclyl is independently substituted with 6 halo. In certain embodiments, the C₁₋₃alkyl is independently substituted with 0, 1, 2, 3, 4, 5, or 6 halo.

In some embodiments, R⁴ is hydrogen, methyl, ethyl, cyclopropyl, or cyclopropyl-methyl. In some embodiments, R⁴ is methyl, ethyl, cyclopropyl, or cyclopropyl-methyl.

In some embodiments, Ring B of formula:

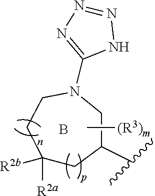

is a group of formula:

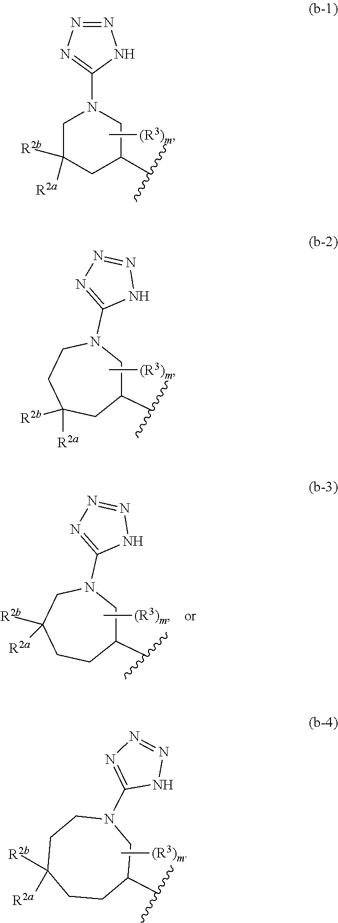

In some embodiments, Ring B is a group of formula:

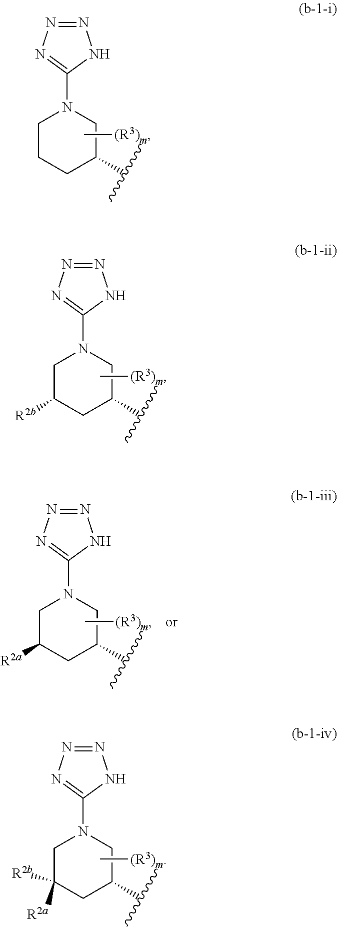

In some embodiments, Ring B is a group of formula (b-1-i), (b-1-ii), (b-1-iii), or (b-1-iv), wherein each instance of R^{2a} and R^{2b} is independently halo, C₁₋₆ alkyl, C₁₋₆ haloalkyl, C₃-C₄ carbocyclyl, or 3-4 membered heterocyclyl.

In some embodiments, Ring B is of formula (b-1), (b-2), (b-3), or (b-4), wherein two R³ groups are joined to form a C₁₋₃ alkylene bridging group or C₁₋₃ haloalkylene bridging group.

In some embodiments, Ring B is a group of formula:

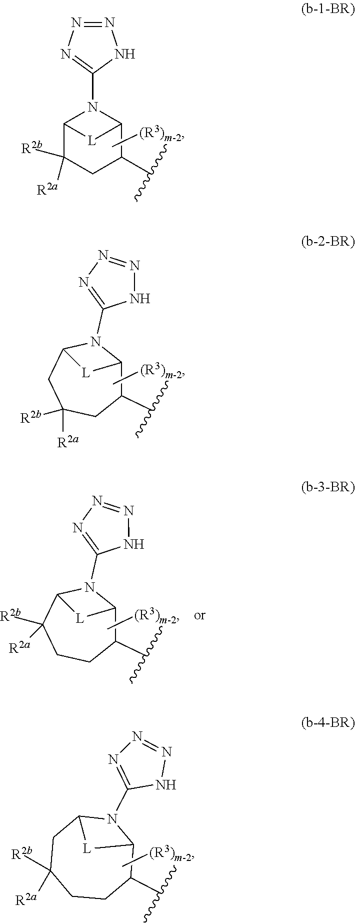

wherein L is a C₁₋₃ alkylene bridging group or C₁₋₃ haloalkylene bridging group.

In some embodiments, L is a C₁₋₃ alkylene bridging group.

In some embodiments, L is a C₁₋₃ haloalkylene bridging group.

In some embodiments, Ring B is a group of formula:

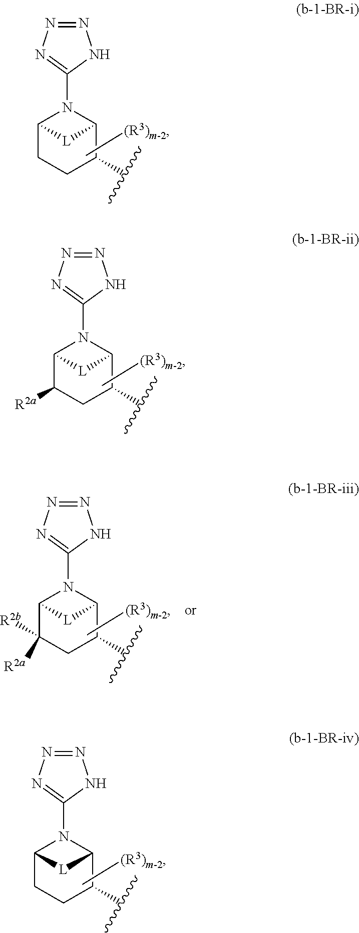

wherein L is a C₁₋₃ alkylene bridging group or C₁₋₃ haloalkylene bridging group.

In some embodiments, Ring B is a group of formula (b-1-BR-i), (b-1-BR-ii), or (b-1-BR-iii), wherein each instance of R^{2a} and R^{2b} is independently halo, C₁₋₆ alkyl, C₁₋₆haloalkyl, C₃-C₄ carbocyclyl, or 3-4 membered heterocyclyl.

In some embodiments, Ring B is a group of formula:

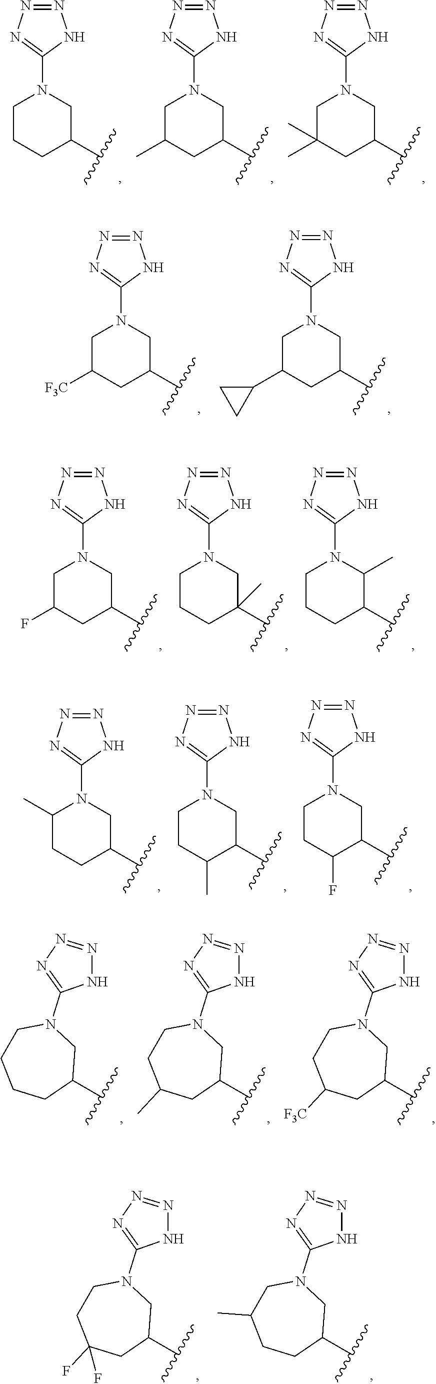

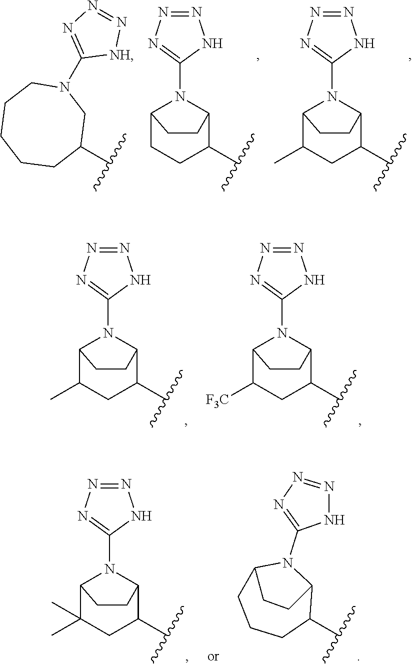

In some embodiments, Ring B is a group of formula:

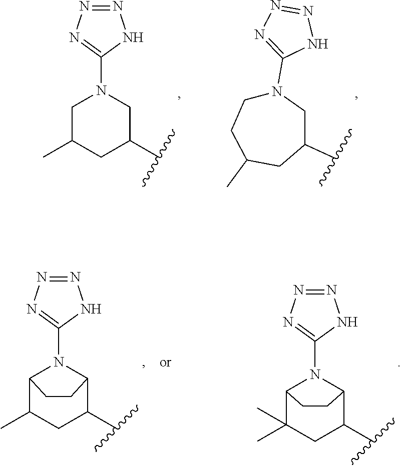 [SMILES: *C1CC(C)CN(c2nnn[nH]2)C1 ; *C1CC(C)CCN(c2nnn[nH]2)C1 ; *C1CC(C)C2CCC1N2c1nnn[nH]1 ; *C1CC(C)(C)C2CCC1N2c1nnn[nH]1]

In some embodiments, Ring B is a group of formula:

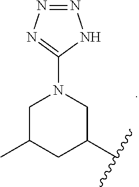 [SMILES: *C1CC(C)CN(c2nnn[nH]2)C1]

In some embodiments, Ring B is a group of formula:

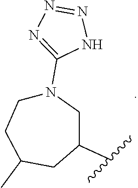 [SMILES: *C1CC(C)CCN(c2nnn[nH]2)C1]

In some embodiments, Ring B is a group of formula:

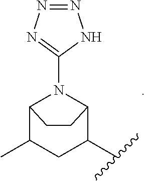 [SMILES: *C1CC(C)C2CCC1N2c1nnn[nH]1]

In some embodiments, Ring B is a group of formula:

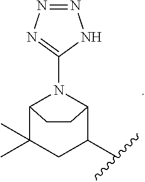 [SMILES: *C1CC(C)(C)C2CCC1N2c1nnn[nH]1]

In some embodiments, Ring B is a group of formula:

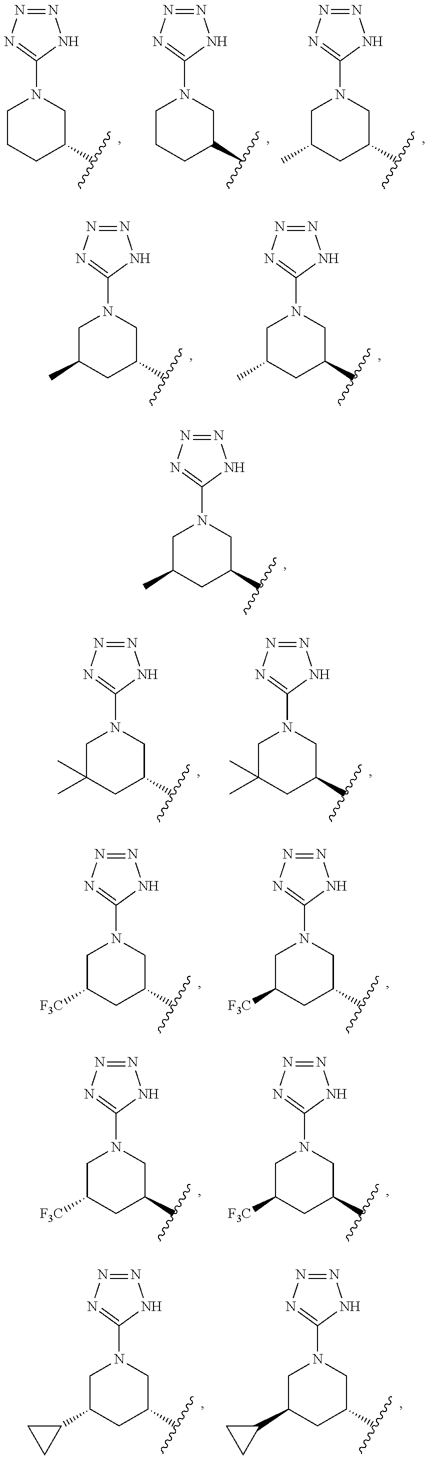

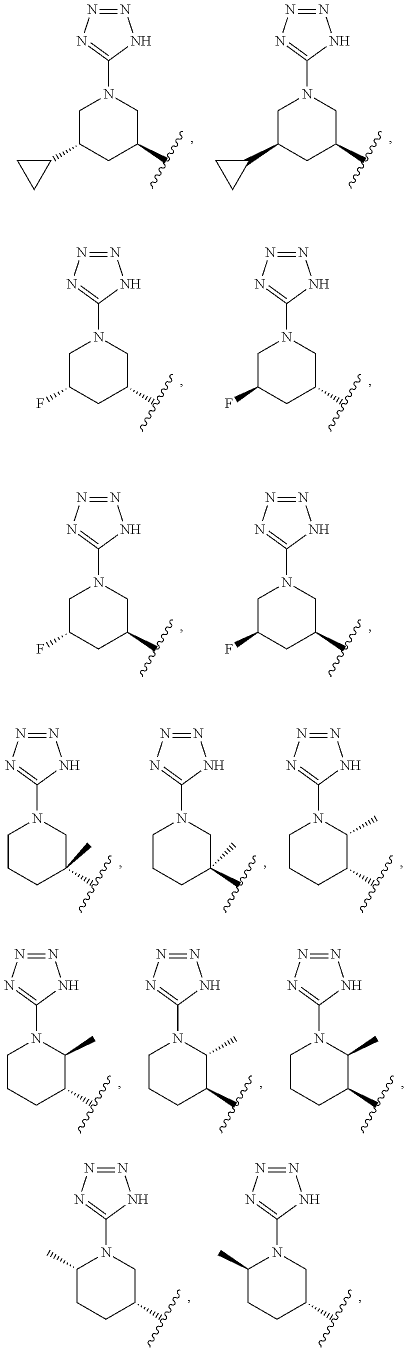

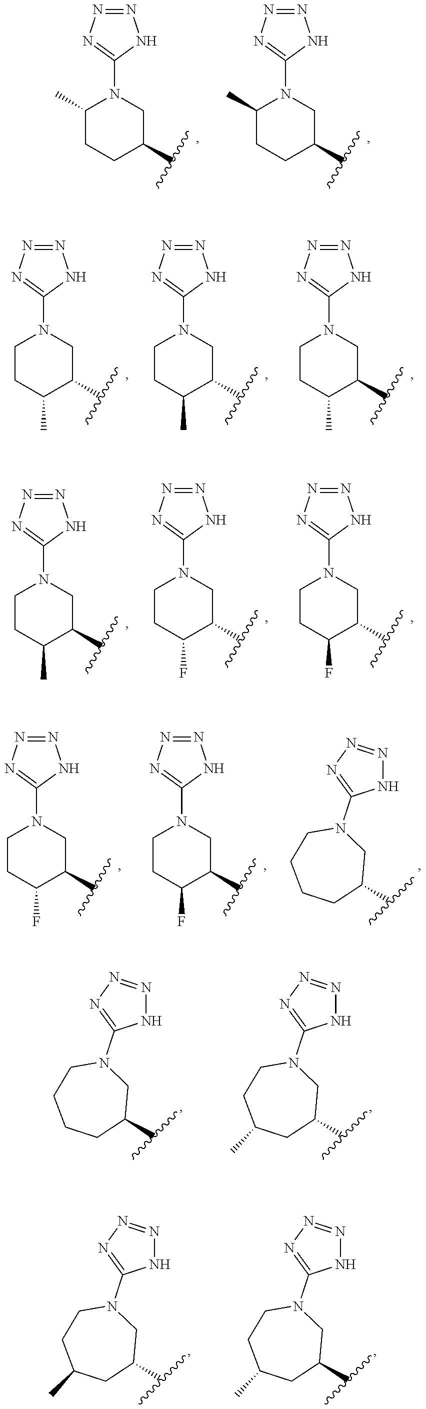

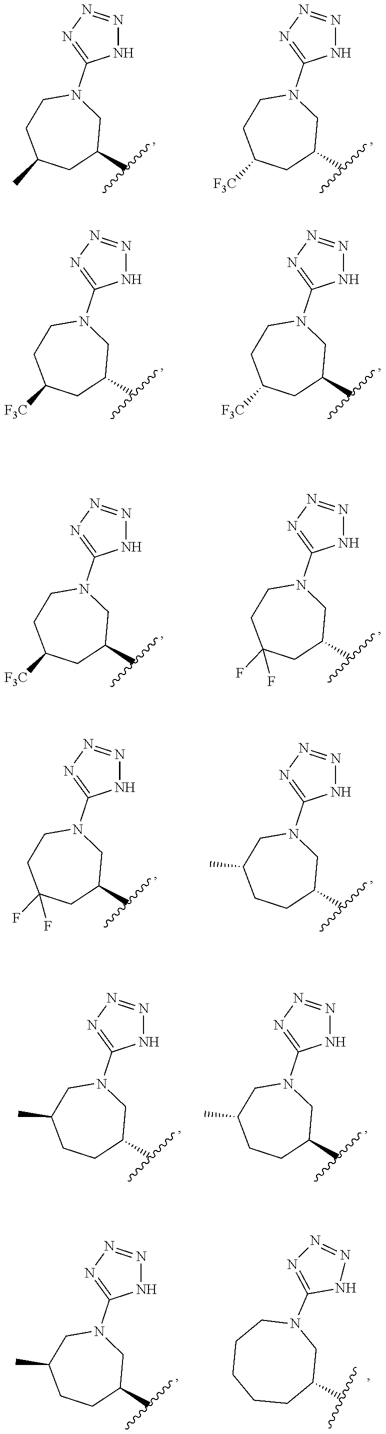

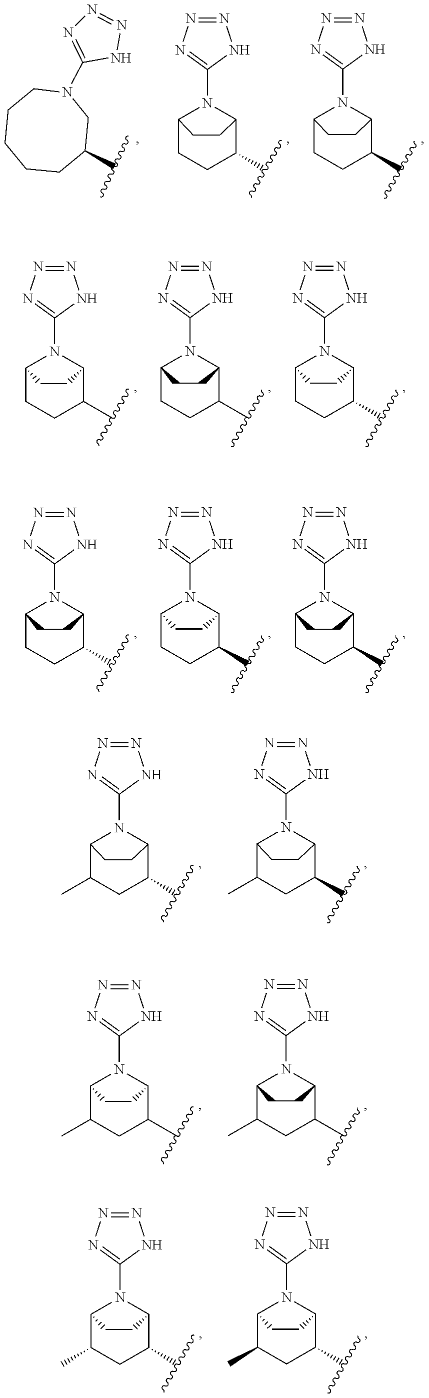

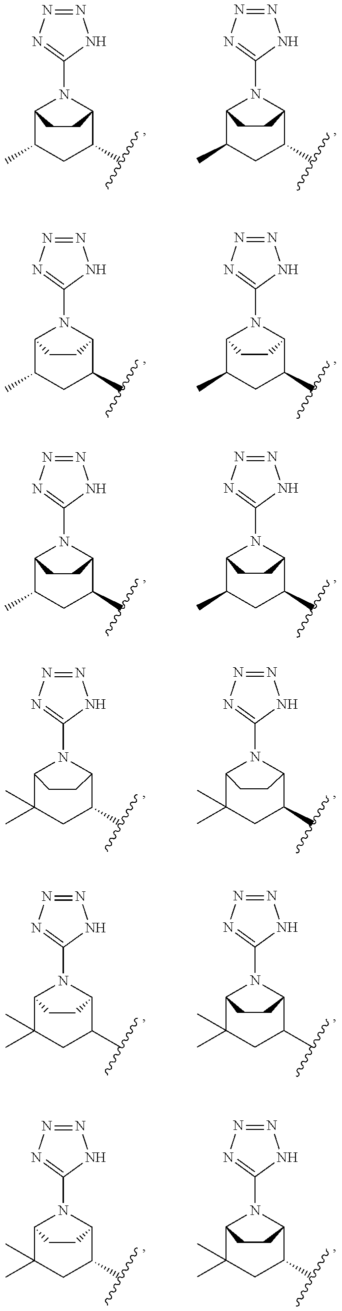

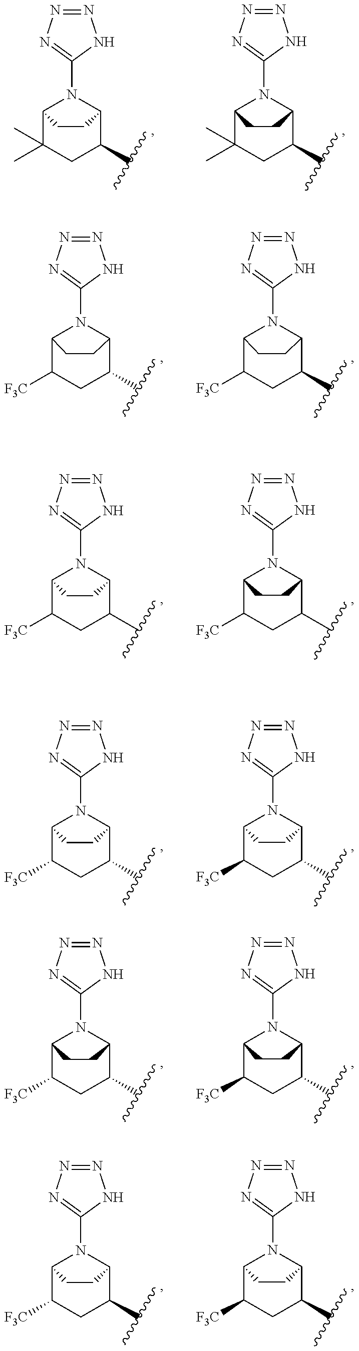

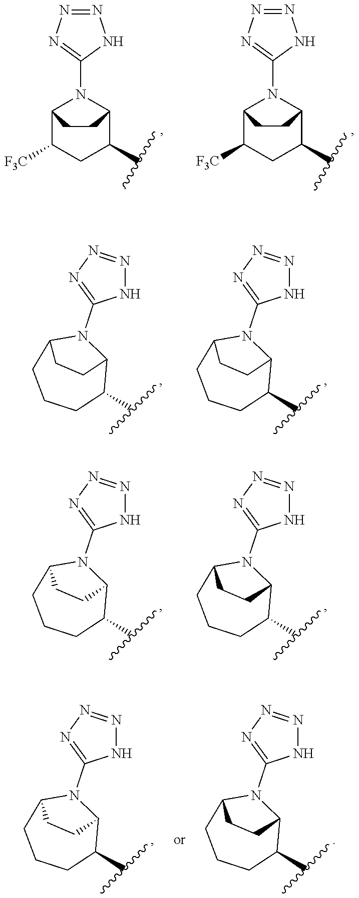

In some embodiments, Ring B is a group of formula:

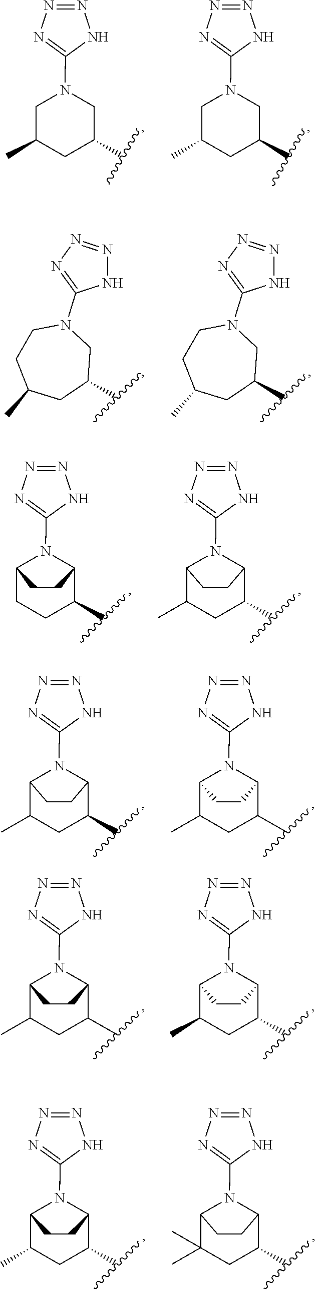

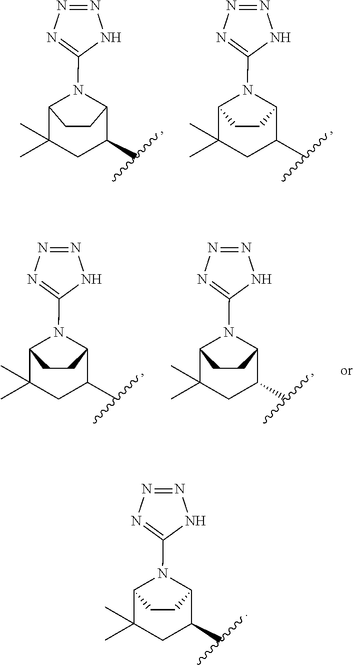 [SMILES: *[C@H]1CC(C)(C)C2CCC1N2c1nnn[nH]1 ; *C1CC(C)(C)[C@@H]2CC[C@H]1N2c1nnn[nH]1 ; *C1CC(C)(C)[C@H]2CC[C@@H]1N2c1nnn[nH]1 ; *[C@@H]1CC(C)(C)[C@H]2CC[C@@H]1N2c1nnn[nH]1 ; *[C@H]1CC(C)(C)[C@@H]2CC[C@H]1N2c1nnn[nH]1]

In some embodiments, Ring B is a group of formula:

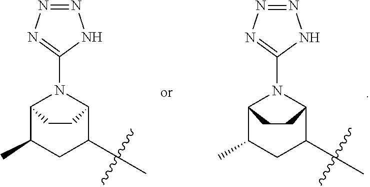 [SMILES: *C1C[C@@H](C)[C@@H]2CC[C@H]1N2c1nnn[nH]1 ; *C1C[C@H](C)[C@H]2CC[C@@H]1N2c1nnn[nH]1]

In some embodiments, Ring B is a group of formula:

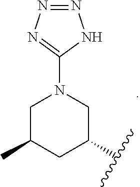 [SMILES: *[C@@H]1C[C@@H](C)CN(c2nnn[nH]2)C1]

In some embodiments, Ring B is a group of formula:

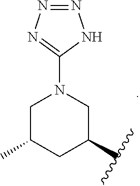 [SMILES: *[C@H]1C[C@H](C)CN(c2nnn[nH]2)C1]

In some embodiments, Ring B is a group of formula:

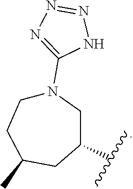 [SMILES: *[C@@H]1C[C@@H](C)CCN(c2nnn[nH]2)C1]

In some embodiments, Ring B is a group of formula:

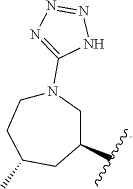 [SMILES: *[C@H]1C[C@H](C)CCN(c2nnn[nH]2)C1]

In some embodiments, Ring B is a group of formula:

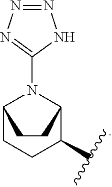 [SMILES: *[C@H]1CC[C@H]2CC[C@@H]1N2c1nnn[nH]1]

In some embodiments, Ring B is a group of formula:

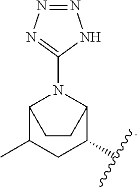 [SMILES: *[C@@H]1CC(C)C2CCC1N2c1nnn[nH]1]

In some embodiments, Ring B is a group of formula:

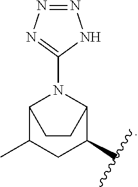 [SMILES: *[C@H]1CC(C)C2CCC1N2c1nnn[nH]1]

In some embodiments, Ring B is a group of formula:

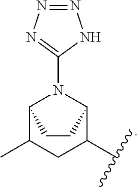 [SMILES: *C1CC(C)[C@@H]2CC[C@H]1N2c1nnn[nH]1]

In some embodiments, Ring B is a group of formula:

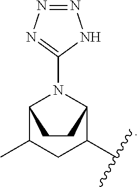 [SMILES: *C1CC(C)[C@H]2CC[C@@H]1N2c1nnn[nH]1]

In some embodiments, Ring B is a group of formula:

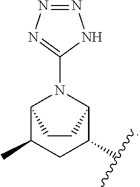 [SMILES: *[C@@H]1C[C@@H](C)[C@@H]2CC[C@H]1N2c1nnn[nH]1]

In some embodiments, Ring B is a group of formula:

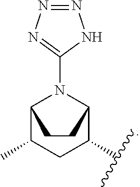 [SMILES: *[C@@H]1C[C@H](C)[C@H]2CC[C@@H]1N2c1nnn[nH]1]

In some embodiments, Ring B is a group of formula:

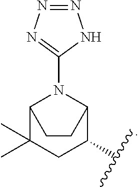 [SMILES: *[C@@H]1CC(C)(C)C2CCC1N2c1nnn[nH]1]

In some embodiments, Ring B is a group of formula:

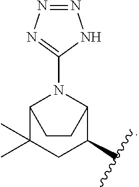 [SMILES: *[C@H]1CC(C)(C)C2CCC1N2c1nnn[nH]1]

In some embodiments, Ring B is a group of formula:

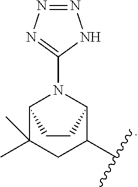 [SMILES: *C1CC(C)(C)[C@@H]2CC[C@H]1N2c1nnn[nH]1]

In some embodiments, Ring B is a group of formula:

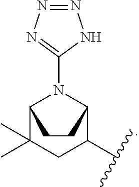 [SMILES: *C1CC(C)(C)[C@H]2CC[C@@H]1N2c1nnn[nH]1]

In some embodiments, Ring B is a group of formula:

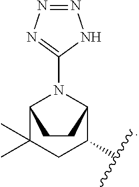 [SMILES: *[C@@H]1CC(C)(C)[C@H]2CC[C@@H]1N2c1nnn[nH]1]

In some embodiments, Ring B is a group of formula:

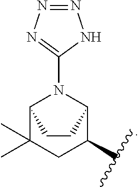 [SMILES: *[C@H]1CC(C)(C)[C@@H]2CC[C@H]1N2c1nnn[nH]1]

### (c) Subgenera

It is understood that, for a compound of the present disclosure, variables Ring A, G₁, G₂, G₃, G₄, R¹, R^{G1}, R^{G2}, R^{G3}, R^{G4}, R^{G5}, R^{G6}, R^{G7}, Ring B, n, p, m, R³, R⁴, R^{2a}, and R^{2b} can each be, where applicable, selected from the groups described herein, and any group described herein for any of variables Ring A, G₁, G₂, G₃, G₄, R¹, R^{G1}, R^{G2}, R^{G3}, R^{G4}, R^{G5}, R^{G6}, R^{G7}, Ring B, n, p, m, R³, R⁴, R^{2a}, and R^{2b} can be combined, where applicable, with any group described herein for one or more of the remainder of variables Ring A, G₁, G₂, G₃, G₄, R, R^{G1}, R^{G2}, R^{G3}, R^{G4}, R^{G5}, R^{G6}, R^{G7}, Ring B, n, p, m, R³, R⁴, R^{2a}, and R^{2b}. Additional exemplary combinations of the above described embodiments are further contemplated herein.

For example, in certain embodiments, provided is a compound of Formula (I-B′), (I-B-a), (I-B′-Bridge), (I-B′″-Bridge), or (I-B-Bridge-a):

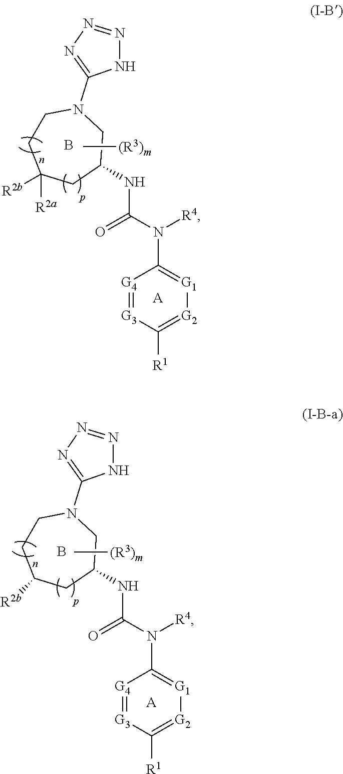

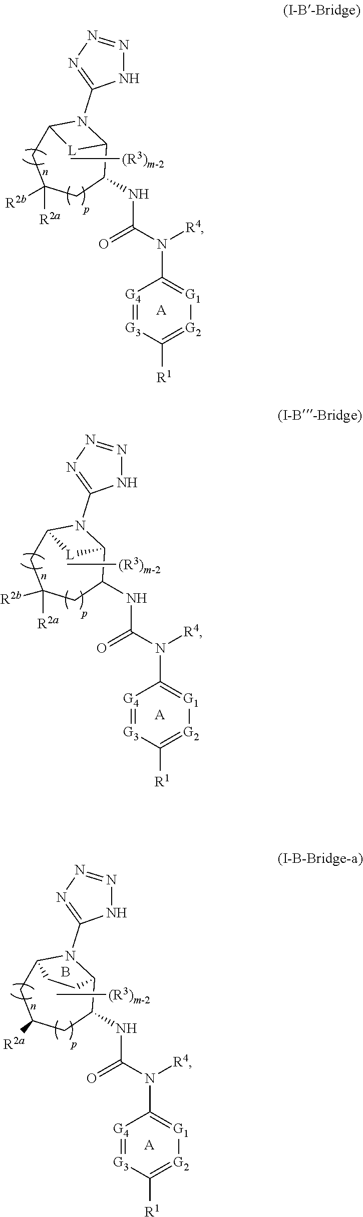

or a pharmaceutically acceptable salt or tautomer thereof.

In some embodiments of Formula (I-B′), (I-B-a), (I-B′-Bridge), (I-B′″-Bridge), or (I-B-Bridge-a), R⁴ is not hydrogen.

In some embodiments of Formula (I-B′), (I-B-a), (I-B′-Bridge), (I-B′″-Bridge), or (I-B-Bridge-a), G₁ is CH, G₂ is CH, G₃ is CH, and G₄ is CH. In some embodiments of Formula (I-B′), (I-B-a), (I-B′-Bridge), (I-B′″-Bridge), or (I-B-Bridge-a), G₁ is CH, G₂ is CH, G₃ is CH, G₄ is CH, and R⁴ is C₁₋₃ alkyl. In some embodiments of Formula (I-B′), (I-B-a), (I-B′-Bridge), (I-B′″-Bridge), or (I-B-Bridge-a), G₁ is CH, G₂ is CH, G₃ is CH, G₄ is CH, and R⁴ is C₃-C₄ carbocyclyl. In some embodiments of Formula (I-B′), (I-B-a), (I-B′-Bridge), (I-B′″-Bridge), or (I-B-Bridge-a), G₁ is CH, G₂ is CH, G₃ is CH, G₄ is CH, and R⁴ is C₃-C₄ carbocyclyl-C₁₋₃ alkyl. In some embodiments of Formula (I-B′), (I-B-a), (I-B′-Bridge), (I-B′″-Bridge), or (I-B-Bridge-a), G₁ is CR^{G1}, G₂ is CH, G₃ is CH, and G₄ is CH. In some embodiments of Formula (I-B′), (I-B-a), (I-B′-Bridge), (I-B′″-Bridge), or (I-B-Bridge-a), G₁ is CR^{G1}, G₂ is CH, G₃ is CH, G₄ is CH, and R⁴ is hydrogen. In some embodiments of Formula (I-B′), (I-B-a), (I-B′-Bridge), (I-B′″-Bridge), or (I-B-Bridge-a), G₁ is CH, G₂ is CR^{G2}, G₃ is CH, and G₄ is CH. In some embodiments of Formula (I-B′), (I-B-a), (I-B′-Bridge), (I-B′″-Bridge), or (I-B-Bridge-a), G₁ is CH, G₂ is CR^{G2}, G₃ is CH, G₄ is CH, and R⁴ is C₁₋₃ alkyl. In some embodiments of Formula (I-B′), (I-B-a), (I-B′-Bridge), (I-B′″-Bridge), or (I-B-Bridge-a), G₁ is CR^{G1}, G₂ is CR^{G2}, G₃ is CH, and G₄ is CH. In some embodiments of Formula (I-B′), (I-B-a), (I-B′-Bridge), (I-B′″-Bridge), or (I-B-Bridge-a), G₁ is CR^{G1}, G₂ is CR^{G2}, G₃ is CH, G₄ is CH, and R⁴ is C₁₋₃ alkyl. In some embodiments of Formula (I-B′), (I-B-a), (I-B′-Bridge), (I-B′″-Bridge), or (I-B-Bridge-a), G₁ is CR^{G1}, G₂ is CH, G₃ is CH, and G₄ is CR^{G4}. In some embodiments of Formula (I-B′), (I-B-a), (I-B′-Bridge), (I-B′″-Bridge), or (I-B-Bridge-a), G₁ is CR^{G1}, G₂ is CH, G₃ is CH, G₄ is CR^{G4}, and R⁴ is C₁₋₃ alkyl. In some embodiments of Formula (I-B′), (I-B-a), (I-B′-Bridge), (I-B′″-Bridge), or (I-B-Bridge-a), G₁ is CR^{G1}, G₂ is CR^{G2}, G₃ is CR^{G3}, and G₄ is CR^{G4}. In some embodiments of Formula (I-B′), (I-B-a), (I-B′-Bridge), (I-B′″-Bridge), or (I-B-Bridge-a), G₁ is CR^{G1}, G₂ is CR^{G2}, G₃ is CR^{G3}, and G₄ is CR^{G4}, and R⁴ is C₁₋₃ alkyl.

In some embodiments of Formula (I-B′), (I-B-a), (I-B′-Bridge), (I-B′″-Bridge), or (I-B-Bridge-a), and any of the above described embodiments of this section, n is 0 and p is 1. In some embodiments, n is 0, p is 1, and at least one of R^{2a} and R^{2b} is C₁₋₆ alkyl. In some embodiments, n is 0, p is 1, and at least one of R^{2a} and R^{2b} is halo. In some embodiments, n is 0, p is 1, and at least one of R^{2a} and R^{2b} is C₁₋₆ haloalkyl. In some embodiments, n is 0, p is 1, and at least one R³ is C₁₋₆ alkyl. In some embodiments, n is 0, p is 1, and at least one R³ is halo. In some embodiments, n is 0, p is 1, and two R³ groups are joined to form a C₁₋₃ alkylene (e.g., ethylene bridge). In some embodiments, n is 0, p is 1, two R³ groups are joined to form a C₁₋₃ alkylene (e.g., ethylene bridge), and at least one of R^{2a} and R^{2b} is C₁₋₆ alkyl.

In some embodiments of Formula (I-B′), (I-B-a), (I-B′-Bridge), (I-B′″-Bridge), or (I-B-Bridge-a), and any of the above described embodiments of this section, R^{G1} is halo and R¹ is —OR^{G5}. In some embodiments, R^{G1} is halo and R¹ is halo. In some embodiments, R^{G1} is halo and R¹ is C₁₋₆haloalkyl.

In some embodiments, R^{G2} is halo and R¹ is halo. In some embodiments, R^{G2} is halo and R¹ is C₁₋₆ haloalkyl. In some embodiments, R^{G2} is halo and R¹ is C₃-C₄ carbocyclyl. In some embodiments, R^{G2} is halo and R¹ is —OR^{G5}. In some embodiments, R^{G2} is —OR^{G6} and R¹ is —OR^{G5}. In some embodiments, R^{G1} is halo, R^{G2} is halo, and R¹ is C₁₋₆haloalkyl. In some embodiments, R^{G1} is halo, R^{G2} is halo, and R¹ is —OR^{G5}. In some embodiments, R^{G1} is halo and R^{G4} is halo. In some embodiments, R^{G1} is halo, R^{G4} is halo, and R¹ is —OR^{G5}. In some embodiments, R^{G1} is halo, R^{G4} is halo, and R¹ is C₃-C₄ carbocyclyl.

In some embodiments of Formula (I-B′), (I-B-a), (I-B′-Bridge), (I-B′″-Bridge), or (I-B-Bridge-a), and any of the above described embodiments of this section, n is 1, p is 1, and at least one of R^{2a} and R^{2b} is C₁₋₆ alkyl. In some embodiments, n is 1, p is 1, and at least one of R^{2a} and R^{2b} is C₁₋₆ haloalkyl. In some embodiments, n is 1, p is 1, and at least one of R^{2a} and R^{2b} is C₁₋₆ alkyl. In some embodiments, n is 1, p is 1, and at least one of R^{2a} and R^{2b} is C₃-C₄ carbocyclyl. In some embodiments, n is 1, p is 1, and at least one of R^{2a} and R^{2b} is halo. In some embodiments, n is 1, p is 1, R^{2a} is H, and R^{2b} is H. In some embodiments, n is 1, p is 1, and two R³ groups are joined to form a C₁₋₃ alkylene (e.g., ethylene bridge). In some embodiments, n is 1, p is 1, two R³ groups are joined to form a C₁₋₃ alkylene (e.g., ethylene bridge), and at least one of R^{2a} and R^{2b} is C₁₋₆ alkyl.

In some embodiments of Formula (I-B′), (I-B-a), (I-B′-Bridge), (I-B′″-Bridge), or (I-B-Bridge-a), Ring A is a ring system wherein:
R¹ is halo, C₁₋₆ haloalkyl, —OR^{G5}, or C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂
or R¹ and G₂, together with the atoms to which they are attached, are joined to form a 5-membered heteroaryl ring independently substituted with 0, 1, 2, or 3 R^{G7};
R^{G1}, R^{G2}, R^{G3}, and R^{G4} are each independently selected from the group consisting of hydrogen, halo, and —OR^{G6};
R^{G5} and R^{G6} are each independently hydrogen or C₁₋₆ haloalkyl; and
each instance of R^{G7} is independently halo, C₁₋₆ alkyl, C₁₋₆ haloalkyl, —OR^{G5}, —SR^{G5}, and —N(R^{G5})₂.

In some embodiments of Formula (I-B′), (I-B-a), (I-B′-Bridge), (I-B′″-Bridge), or (I-B-Bridge-a), Ring A is a ring system wherein:
R¹ is Cl, cyclopropyl, CF₃, CF₂H, OCF₃, or OCF₂H,
or R¹ and G₂, together with the atoms to which they are attached, are joined to form oxazole, isoxazole, pyrazole, or imidazole; and
R^{G1}, R^{G2}, R^{G3}, and R^{G4} are each independently selected from the group consisting of H, F, Cl, OH, and OCF₂H.

In some embodiments of Formula (I-B′), (I-B-a), (I-B′-Bridge), (I-B′″-Bridge), or (I-B-Bridge-a), Ring B is a ring system wherein:
each instance of R^{2a} and R^{2b} is independently hydrogen or C₁₋₆ alkyl;
two R³ groups are joined to form a C₁₋₃ alkylene bridging group; and
R⁴ is C₁₋₃ alkyl, C₃-C₄ carbocyclyl, or C₃-C₄ carbocyclyl-C₁₋₃ alkyl, wherein the alkyl and carbocyclyl are each independently substituted with 0, 1, 2, 3, 4, 5, or 6 halo.

In some embodiments of Formula (I-B′), (I-B-a), (I-B′-Bridge), (I-B′″-Bridge), or (I-B-Bridge-a), Ring B is a ring system wherein:
each instance of R^{2a} and R^{2b} is independently hydrogen or methyl;
two R³ groups are joined to form an ethylene bridging group; and
R⁴ is methyl, ethyl, cyclopropyl, or cyclopropyl-methyl.

In some embodiments of Formula (I-B′), (I-B-a), (I-B′-Bridge), (I-B′″-Bridge), or (I-B-Bridge-a), R⁴ is methyl. In certain embodiments, R⁴ is ethyl.

In certain embodiments of Formula (I-B-a), wherein n is 0, p is 1, m is 0 (where R³ is absent), G₁ is CR^{G1}, G₂ is CR^{G2}, G₃ is CH, and G₄ is CR^{G4}, provided is a compound of Formula (II-B-a1):

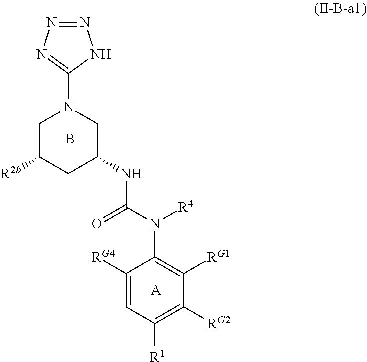

or a pharmaceutically acceptable salt or tautomer thereof. In certain embodiments, R^{2b} and R⁴ are not hydrogen. In certain embodiments, R⁴ is methyl or ethyl. In certain embodiments, R^{2b} is methyl. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen or halogen (e.g., fluoro or chloro). In certain embodiments, R^{G1} is hydrogen or fluoro. In certain embodiments, R^{G1} fluoro. In certain embodiments, R^{G2} is hydrogen, fluoro, or —OR^{G6} wherein R^{G6} is C₁₋₃ alkyl or C₁₋₃ haloalkyl. In certain embodiments, R^{G4} is hydrogen or fluoro. In certain embodiments, R^{G1} is fluoro, R^{G2} is hydrogen, R^{G4} is hydrogen, and R¹ is C₁₋₃ alkyl, C₁₋₃ haloalkyl or —OR^{G5} wherein R^{G5} is C₁₋₃ alkyl or C₁₋₃ haloalkyl.

In certain embodiments of Formula (I-B-a), wherein n is 1, p is 1, m is 0 (where R³ is absent), G₁ is CR^{G1}, G₂ is CR^{G2}, G₃ is CH, and G₄ is CR^{G4}, provided is a compound of Formula (II-B-a2):

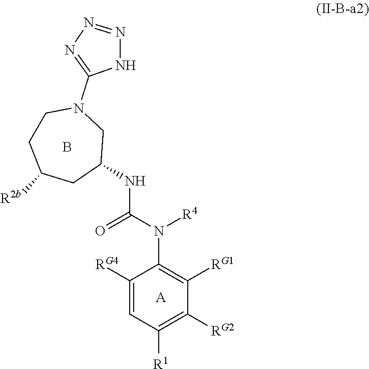

or a pharmaceutically acceptable salt or tautomer thereof. In certain embodiments, R^{2b} and R⁴ are not hydrogen. In certain embodiments, R⁴ is methyl or ethyl. In certain embodiments, R^{2b} is methyl. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen or halogen (e.g., fluoro or chloro). In certain embodiments, R^{G1} is hydrogen or fluoro. In certain embodiments, R^{G1} fluoro. In certain embodiments, R^{G2} is hydrogen, fluoro, or —OR^{G6} wherein R^{G6} is C₁₋₃ alkyl or C₁₋₃ haloalkyl. In certain embodiments, R^{G4} is hydrogen or fluoro. In certain embodiments, R^{G1} is fluoro, R^{G2} is hydrogen, R^{G4} is hydrogen, and R¹ is C₁₋₃ alkyl, C₁₋₃ haloalkyl or —OR^{G5} wherein R^{G5} is C₁₋₃ alkyl or C₁₋₃ haloalkyl.

In certain embodiments of Formula (I-B-Bridge-a), wherein n is 0, p is 1, m is 0 (where an additional R³ is absent), G₁ is CR^{G1}, G₂ is CR^{G2}, G₃ is CH, and G₄ is CR^{G4}, provided is a compound of Formula (II-B-Bridge-a):

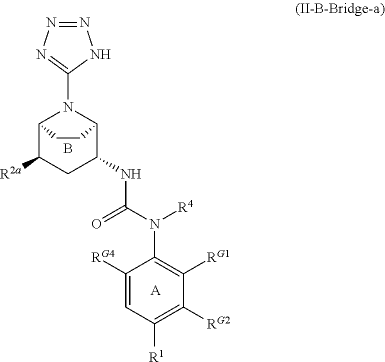

or a pharmaceutically acceptable salt or tautomer thereof. In certain embodiments, R^{2a} and R⁴ are not hydrogen. In certain embodiments, R⁴ is methyl or ethyl. In certain embodiments, R^{2a} is methyl. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen or halogen (e.g., fluoro or chloro). In certain embodiments, R^{G1} is hydrogen or fluoro. In certain embodiments, R^{G1} fluoro. In certain embodiments, R^{G2} is hydrogen, fluoro, or —OR^{G6} wherein R^{G6} is C₁₋₃ alkyl or C₁₋₃ haloalkyl. In certain embodiments, R^{G4} is hydrogen or fluoro. In certain embodiments, R^{G1} is fluoro, R^{G2} is hydrogen, R^{G4} is hydrogen, and R¹ is C₁-3 alkyl, C₁₋₃ haloalkyl or —OR^{G5} wherein R^{G5} is C₁₋₃ alkyl or C₁₋₃ haloalkyl.

In some embodiments, the compound of Formula (I-B) is selected from any one of the compounds of Table 1 or Table 2, or a pharmaceutically acceptable salt or tautomer thereof.

In some embodiments, the compound of Formula (I-B) is a pharmaceutically acceptable salt of any one of the compounds of Table 1 or Table 2 or tautomer thereof.

In some embodiments, the compound of Formula (I-B) is a free base selected from any one of the compounds of Table 1 or Table 2 or tautomer thereof.

The below Tables 1 and 2 also provide the location of the compound in the Examples (Ex) by Example Number (Ex) or as provided in Table A (TA) of the Examples. The Asterix (*) next to the Compound Number (#) signifies that arbitrary stereochemistry has been assigned.

TABLE 1
Compounds of Formula (I-B)

| Ex | # | Structure |
|---|---|---|
| 1 | 2A | 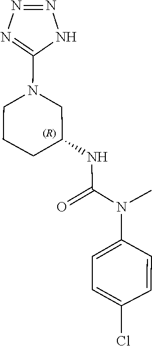 |
| | | (R)-3-(1-(1H-tetrazol-5- |
| | | yl)piperidin-3-yl)-1-(4- |
| | | chlorophenyl)-1-methylurea |
| 1 | 2B | 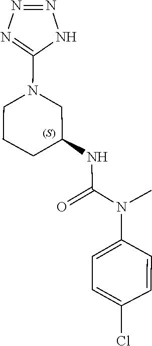 |
| | | (S)-3-(1-(1H-tetrazol-5- |
| | | yl)piperidin-3-yl)-1-(4- |
| | | chlorophenyl)-1-methylurea |
| TA | 3A | 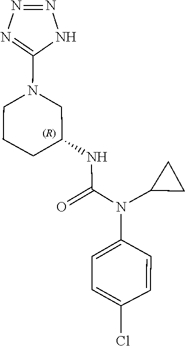 |
| | | (R)-3-(1-(1H-tetrazol-5- |
| | | yl)piperidin-3-yl)-1-(4- |
| | | chlorophenyl)-1-cyclopropylurea |
| TA | 3B | 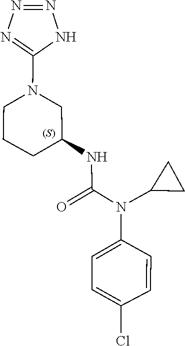 |
| | | (S)-3-(1H-tetrazol-5- |
| | | yl)piperidin-3-yl)-1-(4- |
| | | chlorophenyl)-1-cyclopropylurea |
| 2 | 4A* | 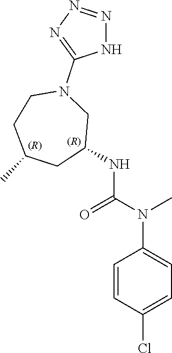 |
| | | 1-(4-chlorophenyl)-1-methyl-3- |
| | | ((3R,5R)-5-methyl-1-(1H- |
| | | tetrazol-5-yl)azepan-3-yl)urea |
| 2 | 4B* | 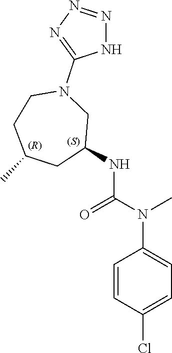 |
| | | 1-(4-chlorophenyl)-1-methyl-3- |
| | | ((3S,5R)-5-methyl-1-(1H- |
| | | tetrazol-5-yl)azepan-3-yl)urea |
| 2 | 4C* | 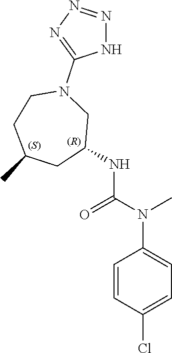 |
| | | 1-(4-chlorophenyl)-1-methyl-3- |
| | | ((3R,5S)-5-methyl-1-(1H- |
| | | tetrazol-5-yl)azepan-3-yl)urea |
| 2 | 4D* | 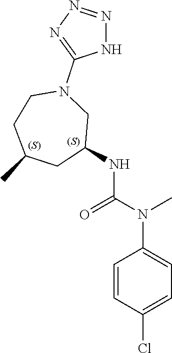 |
| | | 1-(4-chlorophenyl)-1-methyl-3- |
| | | ((3S,5S)-5-methyl-1-(1H- |
| | | tetrazol-5-yl)azepan-3-yl)urea |
| TA | 5A* | 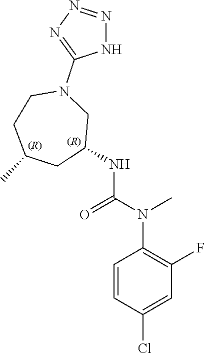 |
| | | 1-(4-chloro-2-fluorophenyl)-1- |
| | | methyl-3-((3R,5R)-5-methyl-1- |
| | | (1H-tetrazol-5-yl)azepan-3- |
| | | yl)urea |
| TA | 5B* | 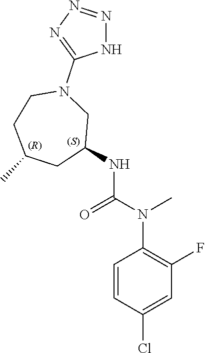 |
| | | 1-(4-chloro-2-fluorophenyl)-1- |
| | | methyl-3-((3S,5R)-5-methyl-1- |
| | | (1H-tetrazol-5-yl)azepan-3- |
| | | yl)urea |
| TA | 5C* | 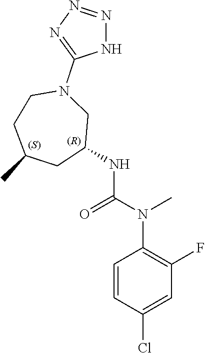 |
| | | 1-(4-chloro-2-fluorophenyl)-1- |
| | | methyl-3-((3R,5S)-5-methyl-1- |
| | | (1H-tetrazol-5-yl)azepan-3- |
| | | yl)urea |
| TA | 5D* | 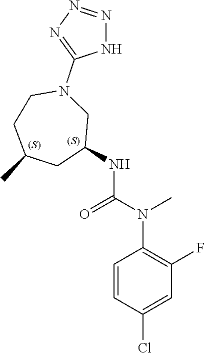 |
| | | 1-(4-chloro-2-fluorophenyl)-1- |
| | | methyl-3-((3S,5S)-5-methyl-1- |
| | | (1H-tetrazol-5-yl)azepan-3- |
| | | yl)urea |
| 3 | 6A | 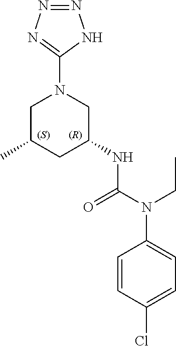 |
| | | 1-(4-chlorophenyl)-1-ethyl-3- |
| | | ((3R,5S)-5-methyl-1-(1H- |
| | | tetrazol-5-yl)piperidin-3-yl)urea |
| 3 | 6B | 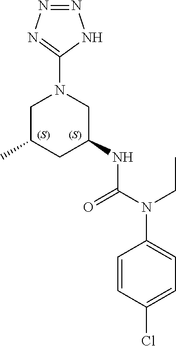 |
| | | 1-(4-chlorophenyl)-1-ethyl-3- |
| | | ((3S,5S)-5-methyl-1-(1H- |
| | | tetrazol-5-yl)piperidin-3-yl)urea |
| 3 | 6C | 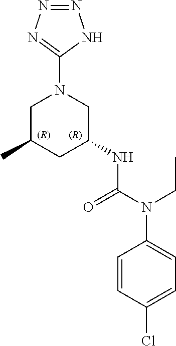 |
| | | 1-(4-chlorophenyl)-1-ethyl-3- |
| | | ((3R,5R)-5-methyl-1-(1H- |
| | | tetrazol-5-yl)piperidin-3-yl)urea |
| 3 | 6D | 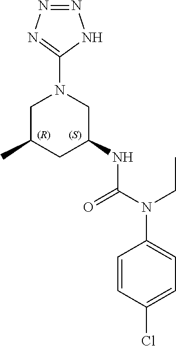 |
| | | 1-(4-chlorophenyl)-1-ethyl-3- |
| | | ((3S,5R)-5-methyl-1-(1H- |
| | | tetrazol-5-yl)piperidin-3-yl)urea |
| TA | 7A* | 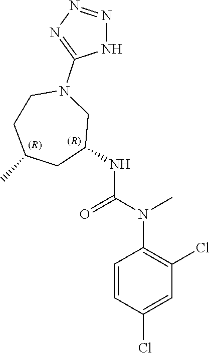 |
| | | 1-(2,4-dichlorophenyl)-1-methyl- |
| | | 3-((3R,5R)-5-methyl-1-(1H- |
| | | tetrazol-5-yl)azepan-3-yl)urea |
| TA | 7B* | 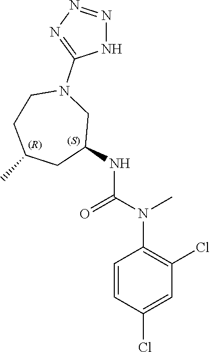 |
| | | 1-(2,4-dichlorophenyl)-1-methyl- |
| | | 3-((3S,5R)-5-methyl-1-(1H- |
| | | tetrazol-5-yl)azepan-3-yl)urea |
| TA | 7C* | 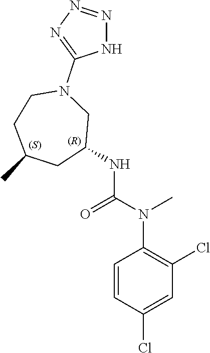 |
| | | 1-(2,4-dichlorophenyl)-1-methyl- |
| | | 3-((3R,5S)-5-methyl-1-(1H- |
| | | tetrazol-5-yl)azepan-3-yl)urea |
| TA | 7D* | 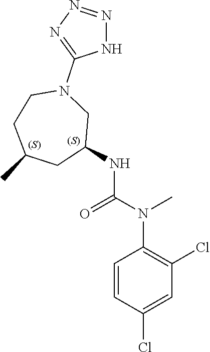 |
| | | 1-(2,4-dichlorophenyl)-1-methyl- |
| | | 3-((3S,5S)-5-methyl-1-(1H- |
| | | tetrazol-5-yl)azepan-3-yl)urea |
| TA | 8A* | 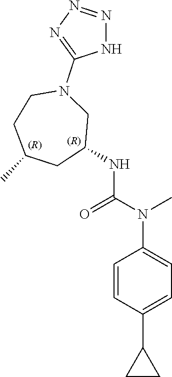 |
| | | 1-(4-cyclopropylphenyl)-1- |
| | | methyl-3-((3R,5R)-5-methyl-1- |
| | | (1H-tetrazol-5-yl)azepan-3- |
| | | yl)urea |
| TA | 8B* | 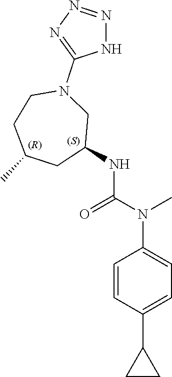 |
| | | 1-(4-cyclopropylphenyl)-1- |
| | | methyl-3-((3S,5R)-5-methyl-1- |
| | | (1H-tetrazol-5-yl)azepan-3- |
| | | yl)urea |
| TA | 8C* | 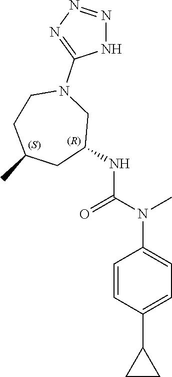 |
| | | 1-(4-cyclopropylphenyl)-1- |
| | | methyl-3-((3R,5S)-5-methyl-1- |
| | | (1H-tetrazol-5-yl)azepan-3- |
| | | yl)urea |
| TA | 8D* | 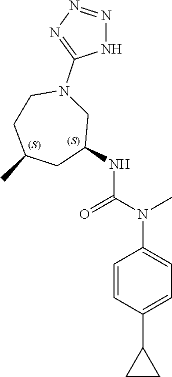 |
| | | 1-(4-cyclopropylphenyl)-1- |
| | | methyl-3-((3S,5S)-5-methyl-1- |
| | | (1H-tetrazol-5-yl)azepan-3- |
| | | yl)urea |
| TA | 9A* | 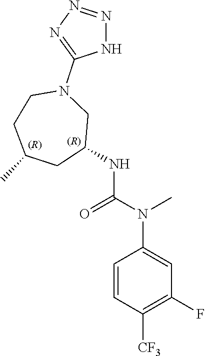 |
| | | 1-(3-fluoro-4- |
| | | (trifluoromethyl)phenyl)-1- |
| | | methyl-3-((3R,5R)-5-methyl-1- |
| | | (1H-tetrazol-5-yl)azepan-3- |
| | | yl)urea |
| TA | 9B* | 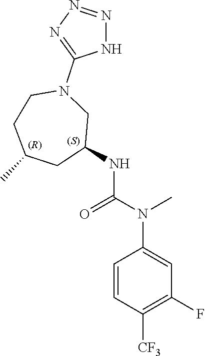 |
| | | 1-(3-fluoro-4- |
| | | (trifluoromethyl)phenyl)-1- |
| | | methyl-3-((3S,5R)-5-methyl-1- |
| | | (1H-tetrazol-5-yl)azepan-3- |
| | | yl)urea |
| TA | 9C* | 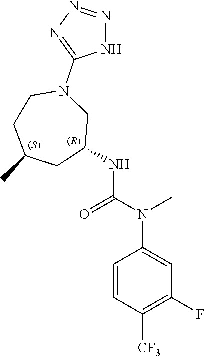 |
| | | 1-(3-fluoro-4- |
| | | (trifluoromethyl)phenyl)-1- |
| | | methyl-3-((3R,5S)-5-methyl-1- |
| | | (1H-tetrazol-5-yl)azepan-3- |
| | | yl)urea |
| TA | 9D* | 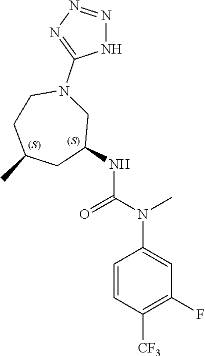 |
| | | 1-(3-fluoro-4- |
| | | (trifluoromethyl)phenyl)-1- |
| | | methyl-3-((3S,5S)-5-methyl-1- |
| | | (1H-tetrazol-5-yl)azepan-3- |
| | | yl)urea |
| TA | 10A* | 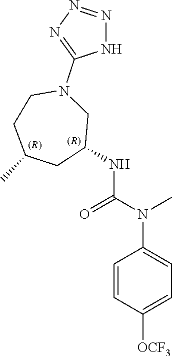 |
| | | 1-methyl-3-((3R,5R)-5-methyl-1- |
| | | (1H-tetrazol-5-yl)azepan-3-yl)-1- |
| | | (4-(trifluoromethoxy)phenyl)urea |
| TA | 10B* | 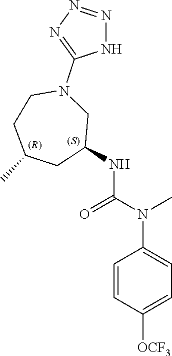 |
| | | 1-methyl-3-((3S,5R)-5-methyl-1- |
| | | (1H-tetrazol-5-yl)azepan-3-yl)-1- |
| | | (4-(trifluoromethoxy)phenyl)urea |
| TA | 10C* | 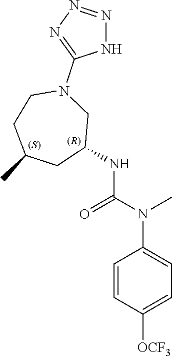 |
| | | 1-methyl-3-((3R,5S)-5-methyl-1- |
| | | (1H-tetrazol-5-yl)azepan-3-yl)-1- |
| | | (4-(trifluoromethoxy)phenyl)urea |
| TA | 10D* | 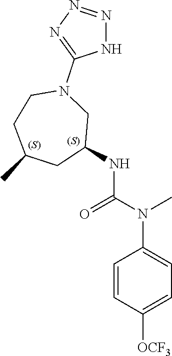 |
| | | 1-methyl-3-((3S,5S)-5-methyl-1- |
| | | (1H-tetrazol-5-yl)azepan-3-yl)-1- |
| | | (4-(trifluoromethoxy)phenyl)urea |
| TA | 11A* | 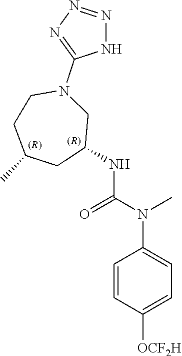 |
| | | 1-(4-(difluoromethoxy)phenyl)- |
| | | 1-methyl-3-((3R,5R)-5-methyl-1- |
| | | (1H-tetrazol-5-yl)azepan-3- |
| | | yl)urea |
| TA | 11B* | 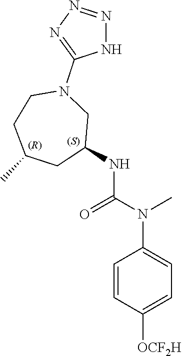 |
| | | 1-(4-(difluoromethoxy)phenyl)- |
| | | 1-methyl-3-((3S,5R)-5-methyl-1- |
| | | (1H-tetrazol-5-yl)azepan-3- |
| | | yl)urea |
| TA | 11C* | 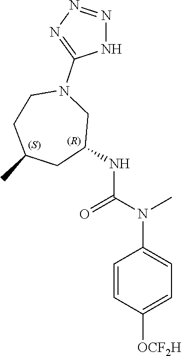 |
| | | 1-(4-(difluoromethoxy)phenyl)- |
| | | 1-methyl-3-((3R,5S)-5-methyl-1- |
| | | (1H-tetrazol-5-yl)azepan-3- |
| | | yl)urea |
| TA | 11D* | 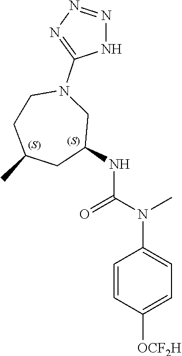 |
| | | 1-(4-(difluoromethoxy)phenyl)- |
| | | 1-methyl-3-((3S,5S)-5-methyl-1- |
| | | (1H-tetrazol-5-yl)azepan-3- |
| | | yl)urea |
| TA | 12A* | 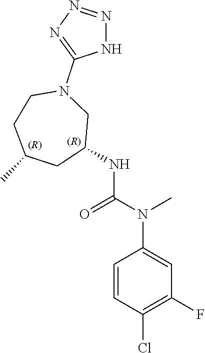 |
| | | 1-(4-chloro-3-fluorophenyl)-1- |
| | | methyl-3-((3R,5R)-5-methyl-1- |
| | | (1H-tetrazol-5-yl)azepan-3- |
| | | yl)urea |
| TA | 12B* | 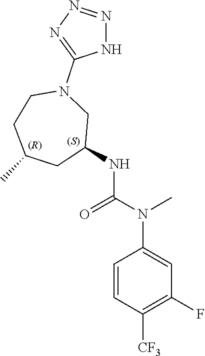 |
| | | 1-(4-chloro-3-fluorophenyl)-1- |
| | | methyl-3-((3S,5R)-5-methyl-1- |
| | | (1H-tetrazol-5-yl)azepan-3- |
| | | yl)urea |
| TA | 12C* | 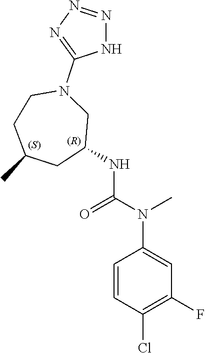 |
| | | 1-(4-chloro-3-fluorophenyl)-1- |
| | | methyl-3-((3R,5S)-5-methyl-1- |
| | | (1H-tetrazol-5-yl)azepan-3- |
| | | yl)urea |
| TA | 12D* | 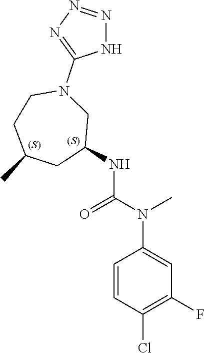 |
| | | 1-(4-chloro-3-fluorophenyl)-1- |
| | | methyl-3-((3S,5S)-5-methyl-1- |
| | | (1H-tetrazol-5-yl)azepan-3- |
| | | yl)urea |
| TA | 13A* | 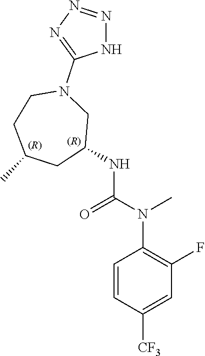 |
| | | 1-(2-fluoro-4- |
| | | (trifluoromethyl)phenyl)-1- |
| | | methyl-3-((3R,5R)-5-methyl-1- |
| | | (1H-tetrazol-5-yl)azepan-3- |
| | | yl)urea |
| TA | 13B* | 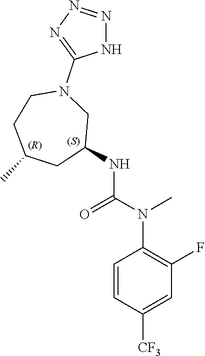 |
| | | 1-(2-fluoro-4- |
| | | (trifluoromethyl)phenyl)-1- |
| | | methyl-3-((3S,5R)-5-methyl-1- |
| | | (1H-tetrazol-5-yl)azepan-3- |
| | | yl)urea |
| TA | 13C* | 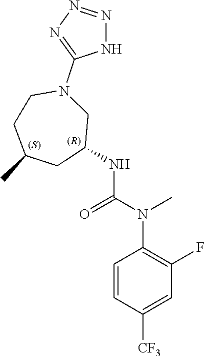 |
| | | 1-(2-fluoro-4- |
| | | (trifluoromethyl)phenyl)-1- |
| | | methyl-3-((3R,5S)-5-methyl-1- |
| | | (1H-tetrazol-5-yl)azepan-3- |
| | | yl)urea |
| TA | 13D* | 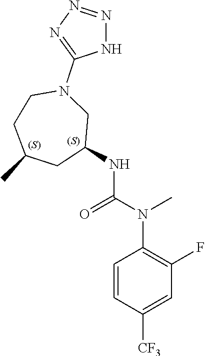 |
| | | 1-(2-fluoro-4- |
| | | (trifluoromethyl)phenyl)-1- |
| | | methyl-3-((3S,5S)-5-methyl-1- |
| | | (1H-tetrazol-5-yl)azepan-3- |
| | | yl)urea |
| TA | 14A* | 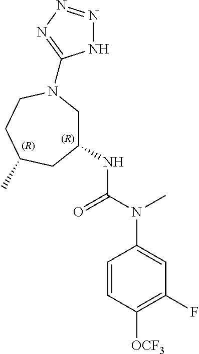 |
| | | 1-(3-fluoro-4- |
| | | (trifluoromethoxy)phenyl)-1- |
| | | methyl-3-((3R,5R)-5-methyl-1- |
| | | (1H-tetrazol-5-yl)azepan-3- |
| | | yl)urea |
| TA | 14B* | 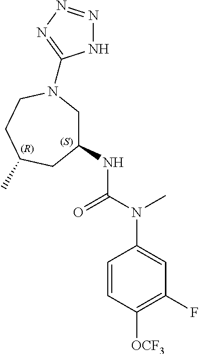 |
| | | 1-(3-fluoro-4- |
| | | (trifluoromethoxy)phenyl)-1- |
| | | methyl-3-((3S,5R)-5-methyl-1- |
| | | (1H-tetrazol-5-yl)azepan-3- |
| | | yl)urea |
| TA | 14C* | 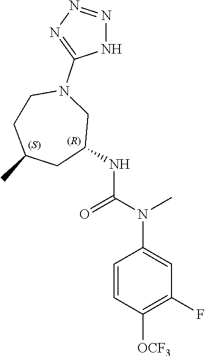 |
| | | 1-(3-fluoro-4- |
| | | (trifluoromethoxy)phenyl)-1- |
| | | methyl-3-((3R,5S)-5-methyl-1- |
| | | (1H-tetrazol-5-yl)azepan-3- |
| | | yl)urea |
| TA | 14D* | 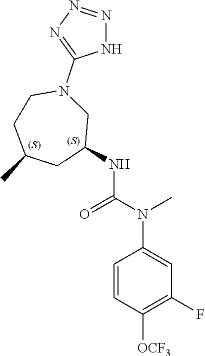 |
| | | 1-(3-fluoro-4- |
| | | (trifluoromethoxy)phenyl)-1- |
| | | methyl-3-((3S,5S)-5-methyl-1- |
| | | (1H-tetrazol-5-yl)azepan-3- |
| | | yl)urea |
| TA | 15A* | 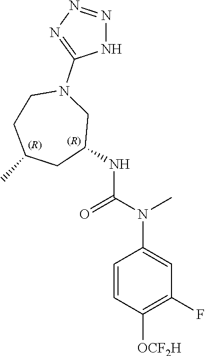 |
| | | 1-(4-(difluoromethoxy)-3- |
| | | fluorophenyl)-1-methyl-3- |
| | | ((3R,5R)-5-methyl-1-(1H- |
| | | tetrazol-5-yl)azepan-3-yl)urea |
| TA | 15B* | 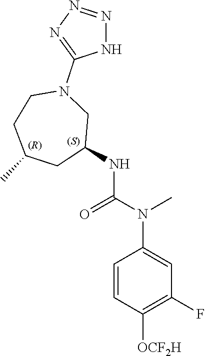 |
| | | 1-(4-(difluoromethoxy)-3- |
| | | fluorophenyl)-1-methyl-3- |
| | | ((3S,5R)-5-methyl-1-(1H- |
| | | tetrazol-5-yl)azepan-3-yl)urea |
| TA | 15C* | 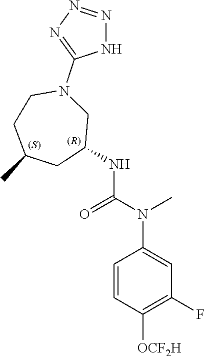 |
| | | 1-(4-(difluoromethoxy)-3- |
| | | fluorophenyl)-1-methyl-3- |
| | | ((3R,5S)-5-methyl-1-(1H- |
| | | tetrazol-5-yl)azepan-3-yl)urea |
| TA | 15D* | 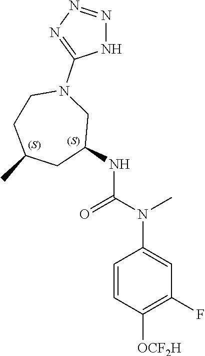 |
| | | 1-(4-(difluoromethoxy)-3- |
| | | fluorophenyl)-1-methyl-3- |
| | | ((3S,5S)-5-methyl-1-(1H- |
| | | tetrazol-5-yl)azepan-3-yl)urea |
| TA | 16A* | 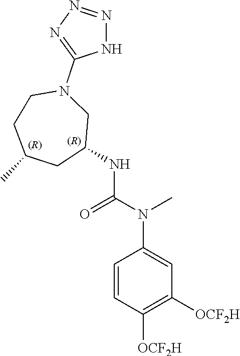 |
| | | 1-(3,4- |
| | | bis(difluoromethoxy)phenyl)-1- |
| | | methyl-3-((3R,5R)-5-methyl-1- |
| | | (1H-tetrazol-5-yl)azepan-3- |
| | | yl)urea |
| TA | 16B* | 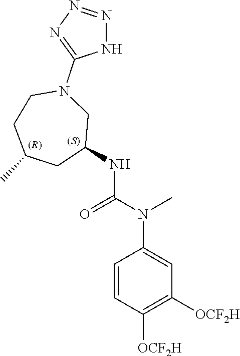 |
| | | 1-(3,4- |
| | | bis(difluoromethoxy)phenyl)-1- |
| | | methyl-3-((3S,5R)-5-methyl-1- |
| | | (1H-tetrazol-5-yl)azepan-3- |
| | | yl)urea |
| TA | 16C* | 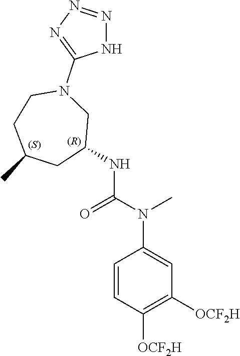 |
| | | 1-(3,4- |
| | | bis(difluoromethoxy)phenyl)-1- |
| | | methyl-3-((3R,5S)-5-methyl-1- |
| | | (1H-tetrazol-5-yl)azepan-3- |
| | | yl)urea |
| TA | 16D* | 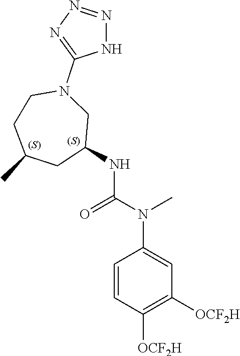 |
| | | 1-(3,4- |
| | | bis(difluoromethoxy)phenyl)-1- |
| | | methyl-3-((3S,5S)-5-methyl-1- |
| | | (1H-tetrazol-5-yl)azepan-3- |
| | | yl)urea |
| TA | 17A* | 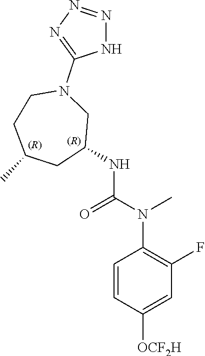 |
| | | 1-(4-(difluoromethoxy)-2- |
| | | fluorophenyl)-1-methyl-3- |
| | | ((3R,5R)-5-methyl-1-(1H- |
| | | tetrazol-5-yl)azepan-3-yl)urea |
| TA | 17B* | 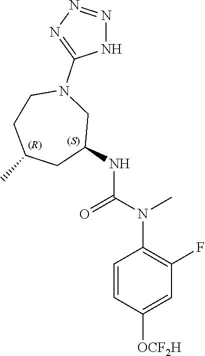 |
| | | 1-(4-(difluoromethoxy)-2- |
| | | fluorophenyl)-1-methyl-3- |
| | | ((3S,5R)-5-methyl-1-(1H- |
| | | tetrazol-5-yl)azepan-3-yl)urea |
| TA | 17C* | 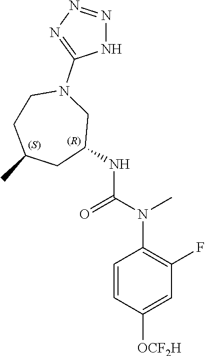 |
| | | 1-(4-(difluroomethoxy)-2- |
| | | fluorophenyl)-1-methyl-3- |
| | | ((3R,5S)-5-methyl-1-(1H- |
| | | tetrazol-5-yl)azepan-3-yl)urea |
| TA | 17D* | 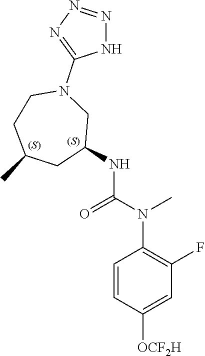 |
| | | 1-(4-(difluoromethoxy)-2- |
| | | fluorophenyl)-1-methyl-3- |
| | | ((3S,5S)-5-methyl-1-(1H- |
| | | tetrazol-5-yl)azepan-3-yl)urea |
| TA | 18A* | 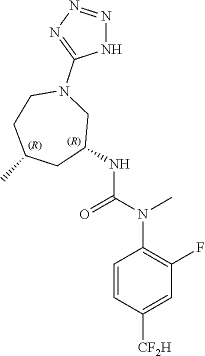 |
| | | 1-(4-(difluoromethyl)-2- |
| | | fluorophenyl)-1-methyl-3- |
| | | ((3R,5R)-5-methyl-1-(1H- |
| | | tetrazol-5-yl)azepan-3-yl)urea |
| TA | 18B* | 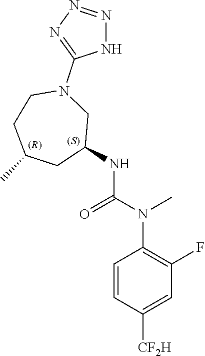 |
| | | 1-(4-(difluoromethyl)-2- |
| | | fluorophenyl)-1-methyl-3- |
| | | ((3S,5R)-5-methyl-1-(1H- |
| | | tetrazol-5-yl)azepan-3-yl)urea |
| TA | 18C* | 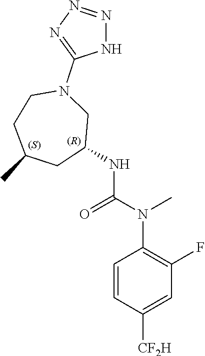 |
| | | 1-(4-(difluoromethyl)-2- |
| | | fluorophenyl)-1-methyl-3- |
| | | ((3R,5S)-5-methyl-1-(1H- |
| | | tetrazol-5-yl)azepan-3-yl)urea |
| TA | 18D* | 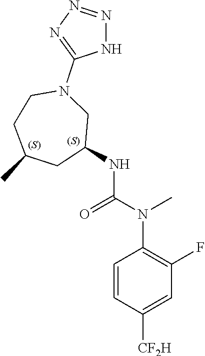 |
| | | 1-(4-(difluoromethyl)-2- |
| | | fluorophenyl)-1-methyl-3- |
| | | ((3S,5S)-5-methyl-1-(1H- |
| | | tetrazol-5-yl)azepan-3-yl)urea |
| TA | 19A* | 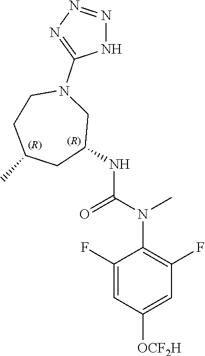 |
| | | 1-(4-(difluoromethoxy)-2,6- |
| | | difluorophenyl)-1-methyl-3- |
| | | ((3R,5R)-5-methyl-1-(1H- |
| | | tetrazol-5-yl)azepan-3-yl)urea |
| TA | 19B* | 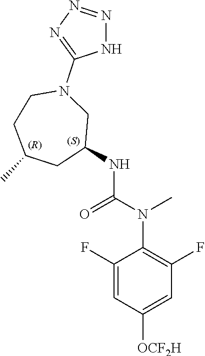 |
| | | 1-(4-(difluoromethoxy)-2,6- |
| | | difluorophenyl)-1-methyl-3- |
| | | ((3S,5R)-5-methyl-1-(1H- |
| | | tetrazol-5-yl)azepan-3-yl)urea |
| TA | 19C* | 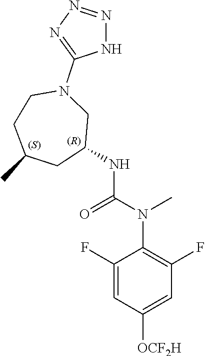 |
| | | 1-(4-(difluoromethoxy)-2,6- |
| | | difluorophenyl)-1-methyl-3- |
| | | ((3R,5S)-5-methyl-1-(1H- |
| | | tetrazol-5-yl)azepan-3-yl)urea |
| TA | 19D* | 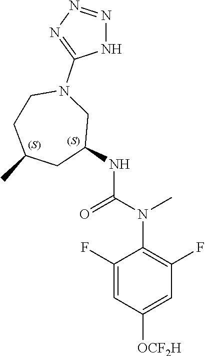 |
| | | 1-(4-(difluoromethoxy)-2,6- |
| | | difluorophenyl)-1-methyl-3- |
| | | ((3S,5S)-5-methyl-1-(1H- |
| | | tetrazol-5-yl)azepan-3-yl)urea |
| TA | 20A* | 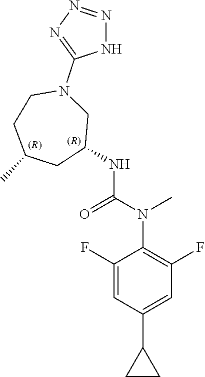 |
| | | 1-(4-cyclopropyl-2,6- |
| | | difluorophenyl)-1-methyl-3- |
| | | ((3R,5R)-5-methyl-1-(1H- |
| | | tetrazol-5-yl)azepan-3-yl)urea |
| TA | 20B* | 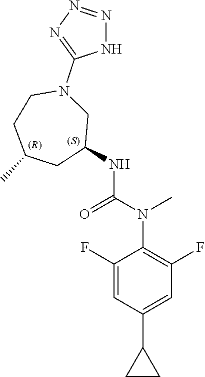 |
| | | 1-(4-cyclopropyl-2,6- |
| | | difluorophenyl)-1-methyl-3- |
| | | ((3S,5R)-5-methyl-1-(1H- |
| | | tetrazol-5-yl)azepan-3-yl)urea |
| TA | 20C* | 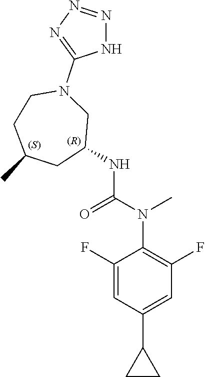 |
| | | 1-(4-cyclopropyl-2,6- |
| | | difluorophenyl)-1-methyl-3- |
| | | ((3R,5S)-5-methyl-1-(1H- |
| | | tetrazol-5-yl)azepan-3-yl)urea |
| TA | 20D* | 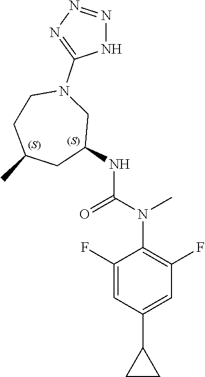 |
| | | 1-(4-cyclopropyl-2,6- |
| | | difluorophenyl)-1-methyl-3- |
| | | ((3S,5S)-5-methyl-1-(1H- |
| | | tetrazol-5-yl)azepan-3-yl)urea |
| TA | 21A* | 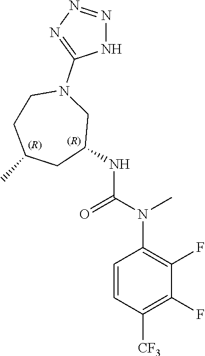 |
| | | 1-(2,3-difluoro-4- |
| | | (trifluoromethyl)phenyl)-1- |
| | | methyl-3-((3R,5R)-5-methyl-1- |
| | | (1H-tetrazol-5-yl)azepan-3- |
| | | yl)urea |
| TA | 21B* | 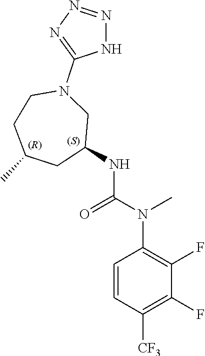 |
| | | 1-(2,3-difluoro-4- |
| | | (trifluoromethyl)phenyl)-1- |
| | | methyl-3-((3S,5R)-5-methyl-1- |
| | | (1H-tetrazol-5-yl)azepan-3- |
| | | yl)urea |
| TA | 21C* | 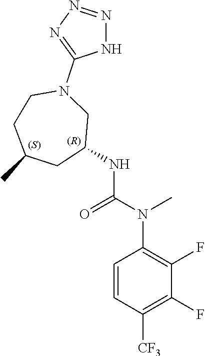 |
| | | 1-(2,3-difluoro-4- |
| | | (trifluoromethyl)phenyl)-1- |
| | | methyl-3-((3R,5S)-5-methyl-1- |
| | | (1H-tetrazol-5-yl)azepan-3- |
| | | yl)urea |
| TA | 21D* | 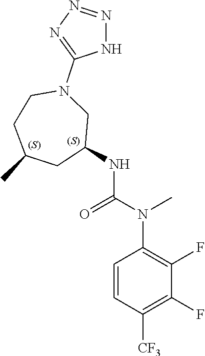 |
| | | 1-(2,3-difluoro-4- |
| | | (trifluoromethyl)phenyl)-1- |
| | | methyl-3-((3S,5S)-5-methyl-1- |
| | | (1H-tetrazol-5-yl)azepan-3- |
| | | yl)urea |
| TA | 22A* | 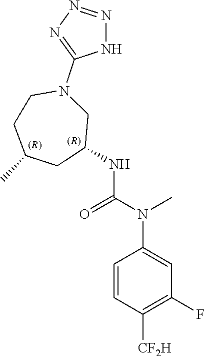 |
| | | 1-(4-(difluoromethyl)-3- |
| | | fluorophenyl)-1-methyl-3- |
| | | ((3R,5R)-5-methyl-1-(1H- |
| | | tetrazol-5-yl)azepan-3-yl)urea |
| TA | 22B* | 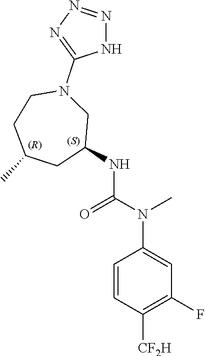 |
| | | 1-(4-(difluoromethyl)-3- |
| | | fluorophenyl)-1-methyl-3- |
| | | ((3S,5R)-5-methyl-1-(1H- |
| | | tetrazol-5-yl)azepan-3-yl)urea |
| TA | 22C* | 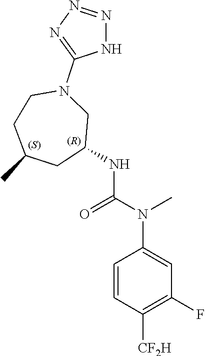 |
| | | 1-(4-(difluoromethyl)-3- |
| | | fluorophenyl)-1-methyl-3- |
| | | ((3R,5S)-5-methyl-1-(1H- |
| | | tetrazol-5-yl)azepan-3-yl)urea |
| TA | 22D* | 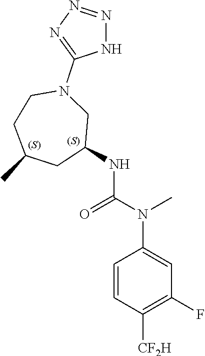 |
| | | 1-(4-(difluoromethyl)-3- |
| | | fluorophenyl)-1-methyl-3- |
| | | ((3S,5S)-5-methyl-1-(1H- |
| | | tetrazol-5-yl)azepan-3-yl)urea |
| TA | 23A* | 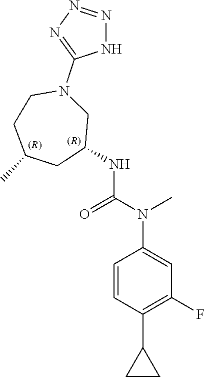 |
| | | 1-(4-cyclopropyl-3- |
| | | fluorophenyl)-1-methyl-3- |
| | | ((3R,5R)-5-methyl-1-(1H- |
| | | tetrazol-5-yl)azepan-3-yl)urea |
| TA | 23B* | 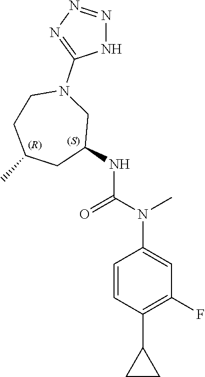 |
| | | 1-(4-cyclopropyl-3- |
| | | fluorophenyl)-1-methyl-3- |
| | | ((3S,5R)-5-methyl-1-(1H- |
| | | tetrazol-5-yl)azepan-3-yl)urea |
| TA | 23C* | 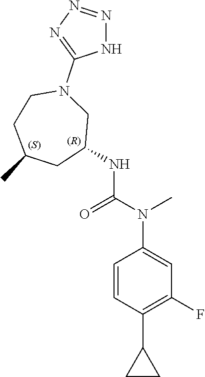 |
| | | 1-(4-cyclopropyl-3- |
| | | fluorophenyl)-1-methyl-3- |
| | | ((3R,5S)-5-methyl-1-(1H- |
| | | tetrazol-5-yl)azepan-3-yl)urea |
| TA | 23D* | 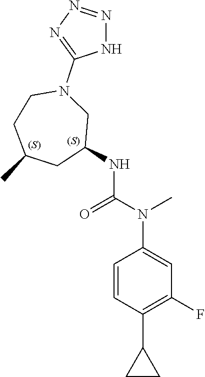 |
| | | 1-(4-cyclopropyl-3- |
| | | fluorophenyl)-1-methyl-3- |
| | | ((3S,5S)-5-methyl-1-(1H- |
| | | tetrazol-5-yl)azepan-3-yl)urea |
| TA | 24A* | 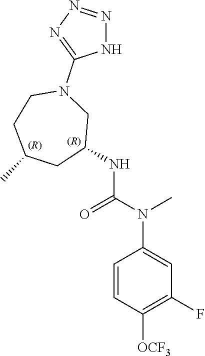 |
| | | 1-(2-fluoro-4- |
| | | (trifluoromethoxy)phenyl)-1- |
| | | methyl-3-((3R,5R)-5-methyl-1- |
| | | (1H-tetrazol-5-yl)azepan-3- |
| | | yl)urea |
| TA | 24B* | 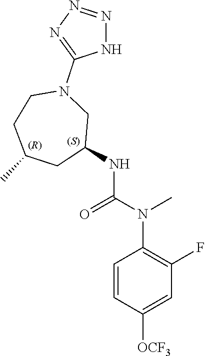 |
| | | 1-(2-fluoro-4- |
| | | (trifluoromethoxy)phenyl)-1- |
| | | methyl-3-((3S,5R)-5-methyl-1- |
| | | 1H-tetrazol-5-yl)azepan-3- |
| | | yl)urea |
| TA | 24C* | 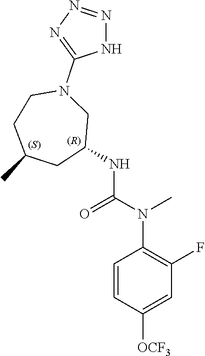 |
| | | 1-(2-fluoro-4- |
| | | (trifluoromethoxy)phenyl)-1- |
| | | methyl-3-((3R,5S)-5-methyl-1- |
| | | (1H-tetrazol-5-yl)azepan-3- |
| | | yl)urea |
| TA | 24D* | 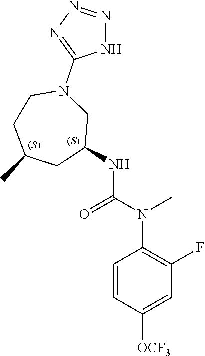 |
| | | 1-(2-fluoro-4- |
| | | (trifluoromethoxy)phenyl)-1- |
| | | methyl-3-((3S,5S)-5-methyl-1- |
| | | (1H-tetrazol-5-yl)azepan-3- |
| | | yl)urea |
| TA | 25A* | 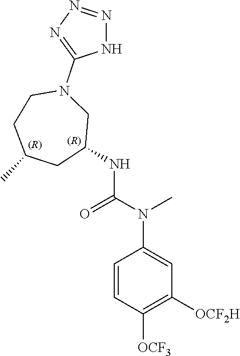 |
| | | 1-(3-(difluoromethoxy)-4- |
| | | (trifluoromethoxy)phenyl)-1- |
| | | methyl-3-((3R,5R)-5-methyl-1- |
| | | (1H-tetrazol-5-yl)azepan-3- |
| | | yl)urea |
| TA | 25B* | 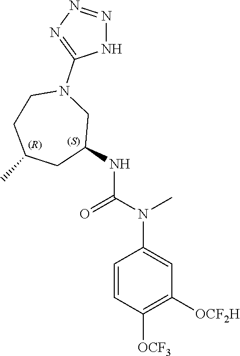 |
| | | 1-(3-(difluoromethoxy)-4- |
| | | (trifluoromethoxy)phenyl)-1- |
| | | methyl-3-((3S,5R)-5-methyl-1- |
| | | (1H-tetrazol-5-yl)azepan-3- |
| | | yl)urea |
| TA | 25C* | 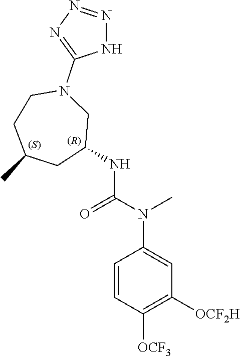 |
| | | 1-(3-(difluoromethoxy)-4- |
| | | (trifluoromethoxy)phenyl)-1- |
| | | methyl-3-((3R,5S)-5-methyl-1- |
| | | (1H-tetrazol-5-yl)azepan-3- |
| | | yl)urea |
| TA | 25D* | 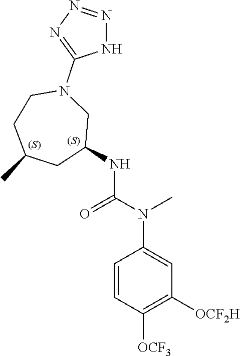 |
| | | 1-(3-(difluoromethoxy)-4- |
| | | (trifluoromethxoy)phenyl)-1- |
| | | methyl-3-((3S,5S)-5-methyl-1- |
| | | (1H-tetrazol-5-yl)azepan-3- |
| | | yl)urea |
| TA | 26A* | 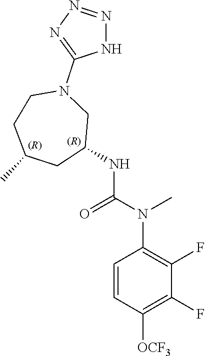 |
| | | 1-(2,3-difluroo-4- |
| | | (trifluoroemthoxy)phenyl)-1- |
| | | methyl-3-((3R,5R)-5-methyl-1- |
| | | (1H-tetrazol-5-yl)azepan-3- |
| | | yl)urea |
| TA | 26B* | 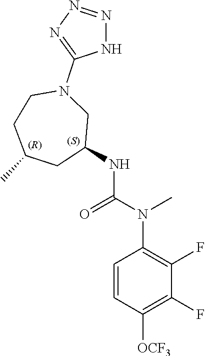 |
| | | 1-(2,3-difluoro-4- |
| | | (trifluoromethoxy)phenyl)-1- |
| | | methyl-3-((3S,5R)-5-methyl-1- |
| | | (1H-tetrazol-5-yl)azepan-3- |
| | | yl)urea |
| TA | 26C* | 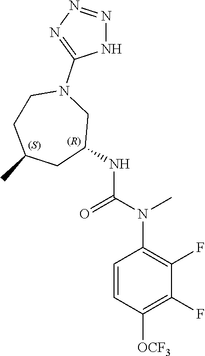 |
| | | 1-(2,3-difluoro-4- |
| | | (trifluoromethoxy)phenyl)-1- |
| | | methyl-3-((3R,5S)-5-methyl-1- |
| | | (1H-tetrazol-5-yl)azepan-3- |
| | | yl)urea |
| TA | 26D* | 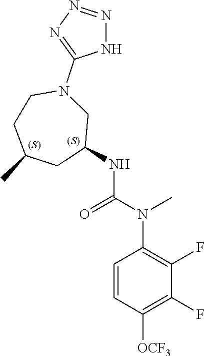 |
| | | 1-(2,3-difluoro-4- |
| | | (trifluoromethoxy)phenyl)-1- |
| | | methyl-3-((3S,5S)-5-methyl-1- |
| | | (1H-tetrazol-5-yl)azepan-3- |
| | | yl)urea |
| TA | 27A* | 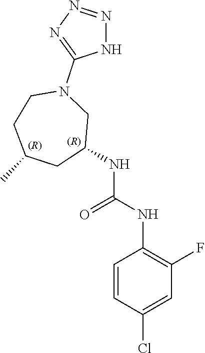 |
| | | 1-(4-chloro-2-fluorophenyl)-3- |
| | | ((3R,5R)-5-methyl-1-(1H- |
| | | tetrazol-5-yl)azepan-3-yl)urea |
| TA | 27B* | 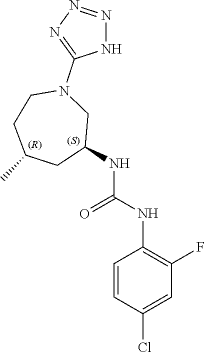 |
| | | 1-(4-chloro-2-fluorophenyl)-3- |
| | | ((3S,5R)-5-methyl-1-(1H- |
| | | tetrazol-5-yl)azepan-3-yl)urea |
| TA | 27C* | 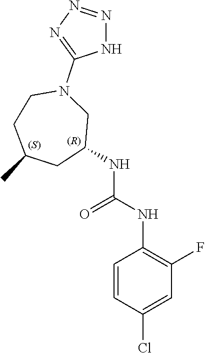 |
| | | 1-(4-chloro-2-fluorophenyl)-3- |
| | | ((3R,5S)-5-methyl-1-(1H- |
| | | tetrazol-5-yl)azepan-3-yl)urea |
| TA | 27D* | 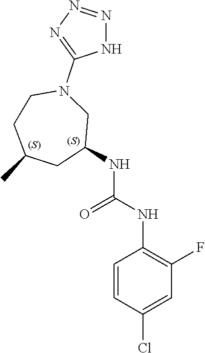 |
| | | 1-(4-chloro-2-fluorophenyl)-3- |
| | | ((3S,5S)-5-methyl-1-(1H- |
| | | tetrazol-5-yl)azepan-3-yl)urea |
| TA | 28A* | 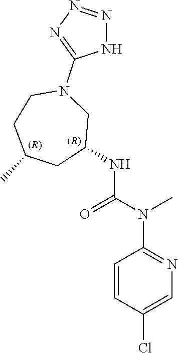 |
| | | 1-(5-chloropyridin-2-yl)-1- |
| | | methyl-3-((3R,5R)-5-methyl-1- |
| | | (1H-tetrazol-5-yl)azepan-3- |
| | | yl)urea |
| TA | 28B* | 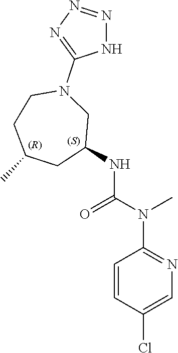 |
| | | 1-(5-chloropyridin-2-yl)-1- |
| | | methyl-3-((3S,5R)-5-methyl-1- |
| | | (1H-tetrazol-5-yl)azepan-3- |
| | | yl)urea |
| TA | 28C* | 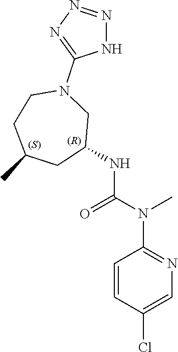 |
| | | 1-(5-chloropyridin-2-yl)-1- |
| | | methyl-3-((3R,5S)-5-methyl-1- |
| | | (1H-tetrazol-5-yl)azepan-3- |
| | | yl)urea |
| TA | 28D* | 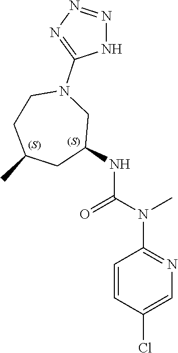 |
| | | 1-(5-chloropyridin-2-yl)-1- |
| | | methyl-3-((3S,5S)-5-methyl-1- |
| | | (1H-tetrazol-5-yl)azepan-3- |
| | | yl)urea |
| TA | 29A* | 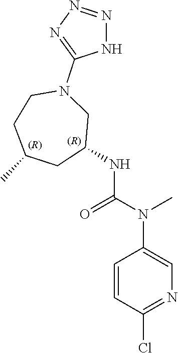 |
| | | 1-(6-chloropyridin-3-yl)-1- |
| | | methyl-3-((3R,5R)-5-methyl-1- |
| | | (1H-tetrazol-5-yl)azepan-3- |
| | | yl)urea |
| TA | 29B* | 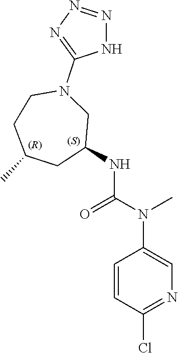 |
| | | 1-(6-chloropyridin-3-yl)-1- |
| | | methyl-3-((3S,5R)-5-methyl-1- |
| | | (1H-tetrazol-5-yl)azepan-3- |
| | | yl)urea |
| TA | 29C* | 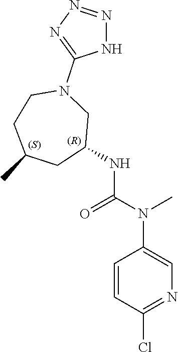 |
| | | 1-(6-chloropyridin-3-yl)-1- |
| | | methyl-3-((3R,5S)-5-methyl-1- |
| | | (1H-tetrazol-5-yl)azepan-3- |
| | | yl)urea |
| TA | 29D* | 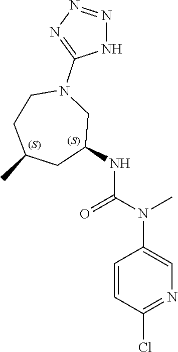 |
| | | 1-(6-chloroyridin-3-yl)-1- |
| | | methyl-3-((3S,5S)-5-methyl-1- |
| | | (1H-tetrazol-5-yl)azepan-3- |
| | | yl)urea |
| TA | 30A* | 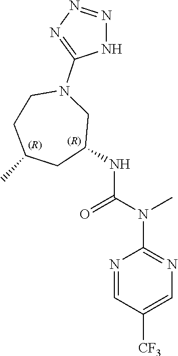 |
| | | 1-methyl-3-((3R,5R)-5-methyl-1- |
| | | (1H-tetrazol-5-yl)azepan-3-yl)-1- |
| | | (5-(trifluoromethyl)pyrimidin-2- |
| | | yl)urea |
| TA | 30B* | 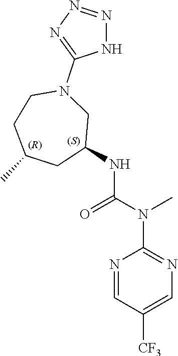 |
| | | 1-methyl-3-((3S,5R)-5-methyl-1- |
| | | (1H-tetrazol-5-yl)azepan-3-yl)-1- |
| | | (5-(trifluoroemthyl)pyrimidin-2- |
| | | yl)urea |
| TA | 30C* | 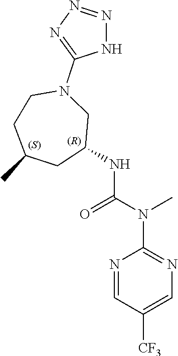 |
| | | 1-methyl-3-((3R,5S)-5-methyl-1- |
| | | (1H-tetrazol-5-yl)azepan-3-yl)-1- |
| | | (5-(trifluoromethyl)pyrimidin-2- |
| | | yl)urea |
| TA | 30D* | 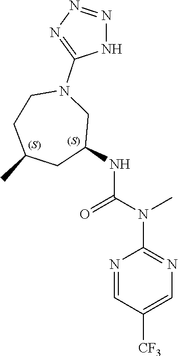 |
| | | 1-methyl-3-((3S,5S)-5-methyl-1- |
| | | (1H-tetrazol-5-yl)azepan-3-yl)-1- |
| | | (5-(trifluoromethyl)pyrimidin-2- |
| | | yl)urea |
| TA | 31A* | 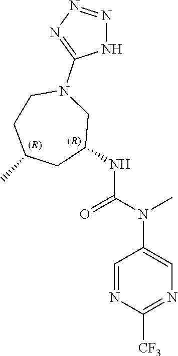 |
| | | 1-methyl-3-((3R,5R)-5-methyl-1- |
| | | (1H-tetrazol-5-yl)azepan-3-yl)-1- |
| | | (2-(trifluoromethyl)pyrimidin-5- |
| | | yl)urea |
| TA | 31B* | 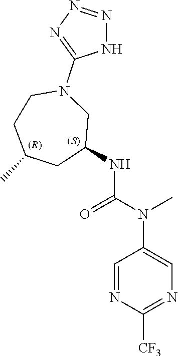 |
| | | 1-methyl-3-((3S,5R)-5-methyl-1- |
| | | (1H-tetrazol-5-yl)azepan-3-yl)-1- |
| | | (2-(trifluoromethyl)pyrimidin-5- |
| | | yl)urea |
| TA | 31C* | 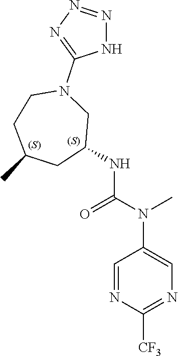 |
| | | 1-methyl-3-((3R,5S)-5-methyl-1- |
| | | (1H-tetrazol-5-yl)azepan-3-yl)-1- |
| | | (2-(trifluromethyl)pyrimidin-5- |
| | | yl)urea |
| TA | 31D* | 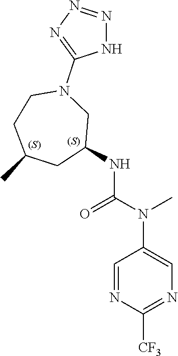 |
| | | 1-methyl-3-((3S,5S)-5-methyl-1- |
| | | (1H-tetrazol-5-yl)azepan-3-yl)-1- |
| | | (2-(trifluoromethyl)pyrimidin-5- |
| | | yl)urea |
| TA | 32A* | 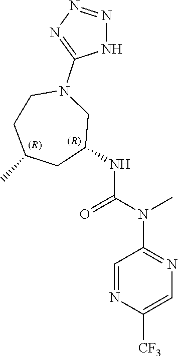 |
| | | 1-methyl-3-((3R,5R)-5-methyl-1- |
| | | (1H-tetrazol-5-yl)azepan-3-yl)-1- |
| | | (5-(trifluoromethyl)pyrazin-2- |
| | | yl)urea |
| TA | 32B* | 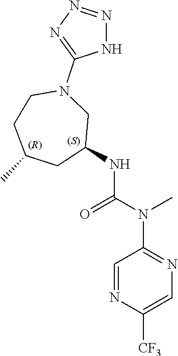 |
| | | 1-methyl-3-((3S,5R)-5-methyl-1- |
| | | (1H-tetrazol-5-yl)azepan-3-yl)-1- |
| | | (5-(trifluoromethyl)pyrazin-2- |
| | | yl)urea |
| TA | 32C* | 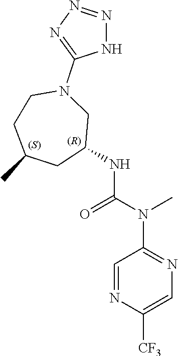 |
| | | 1-methyl-3-((3R,5S)-5-methyl-1- |
| | | (1H-tetrazol-5-yl)azepan-3-yl)-1- |
| | | (5-(trifluoromethyl)pyrazin-2- |
| | | yl)urea |
| TA | 32D* | 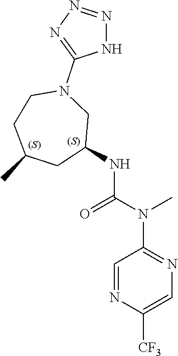 |
| | | 1-methyl-3-((3S,5S)-5-methyl-1- |
| | | (1H-tetrazol-5-yl)azepan-3-yl)-1- |
| | | (5-(trifluoromethyl)pyrazin-2- |
| | | yl)urea |
| TA | 33A* | 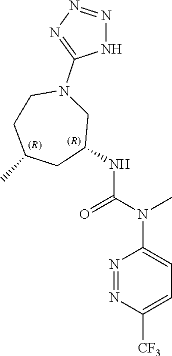 |
| | | 1-methyl-3-((3R,5R)-5-methyl-1- |
| | | (1H-tetrazol-5-yl)azepan-3-yl)-1- |
| | | (6-(trifluoromethyl)pyridazin-3- |
| | | yl)urea |
| TA | 33B* | 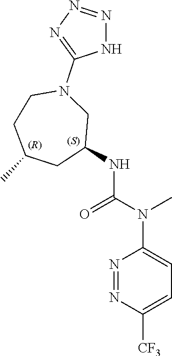 |
| | | 1-methyl-3-((3S,5R)-5-methyl-1- |
| | | (1H-tetrazol-5-yl)azepan-3-yl)-1- |
| | | (6-(trifluoromethyl)pyridazin-3- |
| | | yl)urea |
| TA | 33C* | 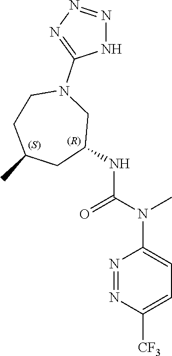 |
| | | 1-methyl-3-((3R,5S)-5-methyl-1- |
| | | (1H-tetrazol-5-yl)azepan-3-yl)-1- |
| | | (6-(trifluoromethyl)pyridazin-3- |
| | | yl)urea |
| TA | 33D* | 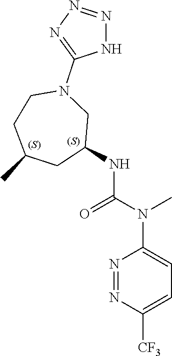 |
| | | 1-methyl-3-((3S,5S)-5-methyl-1- |
| | | (1H-tetrazol-5-yl)azepan-3-yl)-1- |
| | | (6-(trifluoromethyl)pyridazin-3- |
| | | yl)urea |
| TA | 34A | 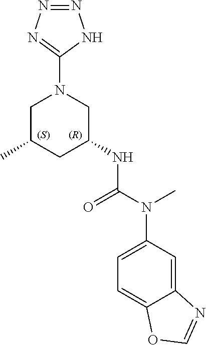 |
| | | 1-(benzo[d]oxazol-5-yl)-1- |
| | | methyl-3-((3R,5S)-5-methyl-1- |
| | | (1H-tetrazol-5-yl)piperidin-3- |
| | | yl)urea |
| TA | 34B | 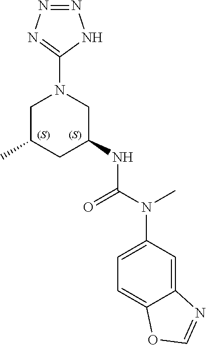 |
| | | 1-(benzo[d]oxazol-5-yl)-1- |
| | | methyl-3-((3S,5S)-5-methyl-1- |
| | | (1H-tetrazol-5-yl)piperidin-3- |
| | | yl)urea |
| TA | 34C | 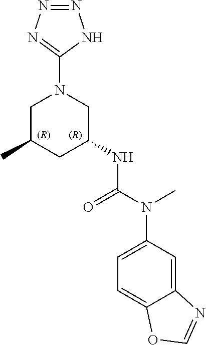 |
| | | 1-(benzo[d]oxazol-5-yl)-1- |
| | | methyl-3-((3R,5R)-5-methyl-1- |
| | | (1H-tetrazol-5-yl)piperidin-3- |
| | | yl)urea |
| TA | 34D | 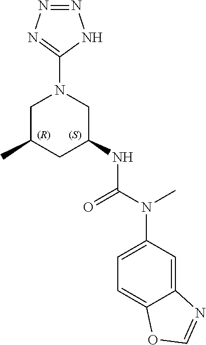 |
| | | 1-(benzo[d]oxaol-5-yl)-1- |
| | | methyl-3-((3S,5R)-5-methyl-1- |
| | | (1H-tetrazol-5-yl)piperidin-3- |
| | | yl)urea |
| TA | 35A | 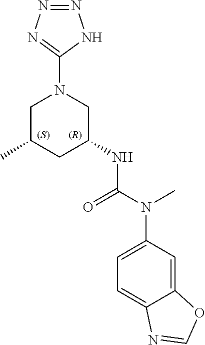 |
| | | 1-(benzo[d]oxazol-6-yl)-1- |
| | | methyl-3-((3R,5S)-5-methyl-1- |
| | | (1H-tetrazol-5-yl)piperidin-3- |
| | | yl)urea |
| TA | 35B | 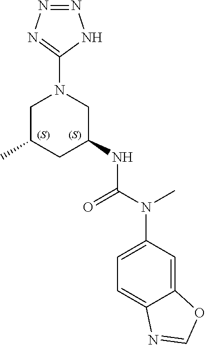 |
| | | 1-(benzo[d]oxazol-6-yl)-1- |
| | | methyl-3-((3S,5S)-5-methyl-1- |
| | | (1H-tetrazol-5-yl)piperidin-3- |
| | | yl)urea |
| TA | 35C | 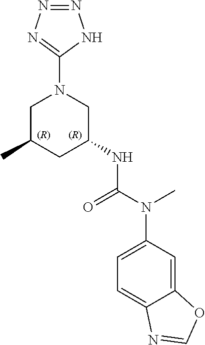 |
| | | 1-(benzo[d]oxazol-6-yl)-1- |
| | | methyl-3-((3R,5R)-5-methyl-1- |
| | | (1H-tetrazol-5-yl)piperidin-3- |
| | | yl)urea |
| TA | 35D | 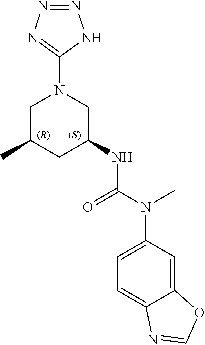 |
| | | 1-(benzo[d]oxazol-6-yl)-1- |
| | | methyl-3-((3S,5R)-5-methyl-1- |
| | | (1H-tetrazol-5-yl)piperidin-3- |
| | | yl)urea |
| TA | 36A | 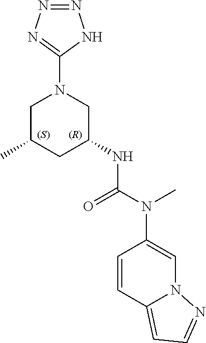 |
| | | 1-methyl-3-((3R,5S)-5-methyl-1- |
| | | (1H-tetrazol-5-yl)piperidin-3-yl)- |
| | | 1-(pyrazolo[1,5-a]pyridin-6- |
| | | yl)urea |
| TA | 36B | 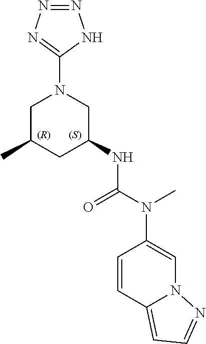 |
| | | 1-methyl-3-((3S,5S)-5-methyl-1- |
| | | (1H-tetrazol-5-yl)piperidin-3-yl)- |
| | | 1-(pyrazolo[1,5-a]pyridin-6- |
| | | yl)urea |
| TA | 36C | 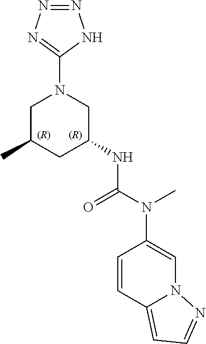 |
| | | 1-methyl-3-((3R,5R)-5-methyl-1- |
| | | (1H-tetrazol-5-yl)piperidin-3-yl)- |
| | | 1-(pyrazolo[1,5-a]pyridin-6- |
| | | yl)urea |
| TA | 36D | 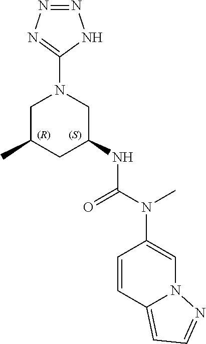 |
| | | 1-methyl-3-((3S,5R)-5-methyl-1- |
| | | (1H-tetrazol-5-yl)piperidin-3-yl)- |
| | | 1-(pyrazolo[1,5-a]pyridin-6- |
| | | yl)urea |
| TA | 37A | 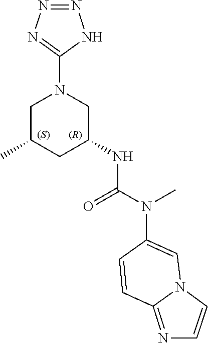 |
| | | 1-(imidazo[1,2-a]pyridin-6-yl)-1- |
| | | methyl-3-((3R,5S)-5-methyl-1- |
| | | (1H-tetrazol-5-yl)piperidin-3- |
| | | yl)urea |
| TA | 37B | 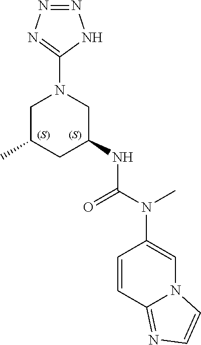 |
| | | 1-(imidazo[1,2-a]pyridin6--yl)-1- |
| | | methyl-3-((3S,5S)-5-methyl-1- |
| | | (1H-tetrazol-5-yl)piperidin-3- |
| | | yl)urea |
| TA | 37C | 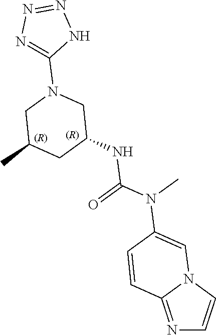 |
| | | 1-(imidazo[1,2-a]pyridin-6-yl)-1- |
| | | methyl-3-((3R,5R)-5-methyl-1- |
| | | (1H-tetrazol-5-yl)piperidin-3- |
| | | yl)urea |
| TA | 37D | 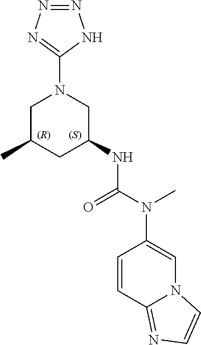 |
| | | 1-(imidazo[1,2-a]pyridin-6-yl)-1- |
| | | methyl-3-((3S,5R)-5-methyl-1- |
| | | (1H-tetrazol-5-yl)piperidin-3- |
| | | yl)urea |
| TA | 38A | 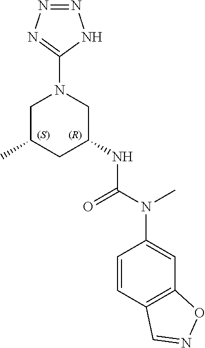 |
| | | 1-(benzo[d]isoxazol-6-yl)-1- |
| | | methyl-3-((3R,5S)-5-methyl-1- |
| | | (1H-tetrazol-5-yl)piperidin-3- |
| | | yl)urea |
| TA | 38B | 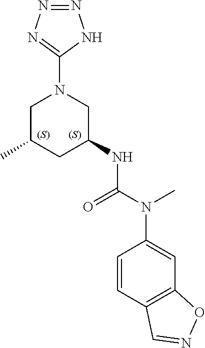 |
| | | 1-(benzo[d]isoxazol-6-yl)-1- |
| | | methyl-3-((3S,5S)-5-methyl-1- |
| | | (1H-tetrazol-5-yl)piperidin-3- |
| | | yl)urea |
| TA | 38C | 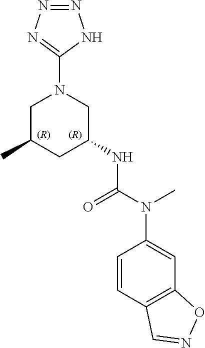 |
| | | 1-(benzo[d]isoxazol-6-yl)-1- |
| | | methyl-3-((3R,5R)-5-methyl-1- |
| | | (1H-tetrazol-5-yl)piperidin-3- |
| | | yl)urea |
| TA | 38D | 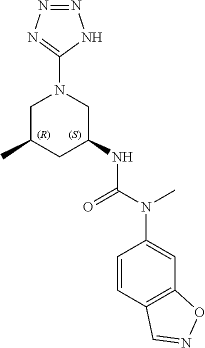 |
| | | 1-(benzo[d]isoxazol-6-yl)-1- |
| | | methyl-3-((3S,5R)-5-methyl-1- |
| | | (1H-tetrazol-5-yl)piperidin-3- |
| | | yl)urea |
| TA | 39A | 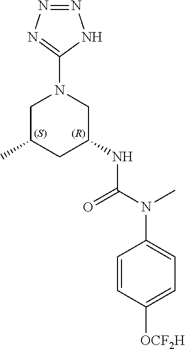 |
| | | 1-(4-(difluoromethxoy)phenyl)- |
| | | 1-methyl-3-((3R,5S)-5-methyl-1- |
| | | (1H-tetrazol-5-yl)piperidin-3- |
| | | yl)urea |
| TA | 39B | 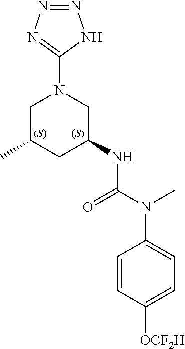 |
| | | 1-(4-(difluoromethoxy)phenyl)- |
| | | 1-methyl-3-((3S,5S)-5-methyl-1- |
| | | (1H-tetrazol-5-yl)piperidin-3- |
| | | yl)urea |
| TA | 39C | 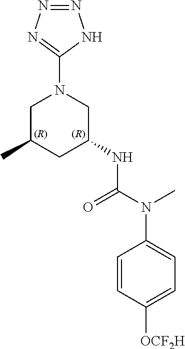 |
| | | 1-(4-(difluoromethoxy)phenyl)- |
| | | 1-methyl-3-((3R,5R)-5-methyl-1- |
| | | (1H-tetrazol-5-yl)piperidin-3- |
| | | yl)urea |
| TA | 39D | 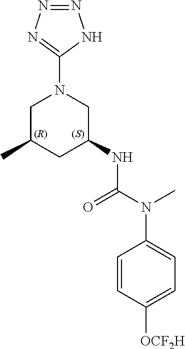 |
| | | 1-(4-(difluoromethoxy)phenyl)- |
| | | 1-methyl-3-((3S,5R)-5-methyl-1- |
| | | (1H-tetrazol-5-yl)piperidin-3- |
| | | yl)urea |
| TA | 40A* | 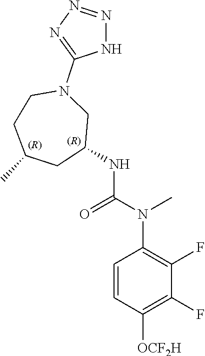 |
| | | 1-(4-(difluoromethoxy)-2,3- |
| | | difluorophenyl)-1-methyl-3- |
| | | ((3R,5R)-5-methyl-1-(1H- |
| | | tetrazol-5-yl)azepan-3-yl)urea |
| TA | 40B* | 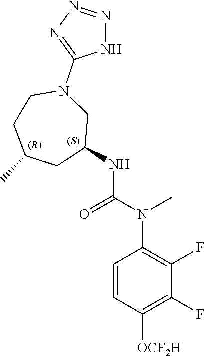 |
| | | 1-(4-(difluoromethoxy)-2,3- |
| | | difluorophenyl)-1-methyl-3- |
| | | ((3S,5R)-5-methyl-1-(1H- |
| | | tetrazol-5-yl)azepan-3-yl)urea |
| TA | 40C* | 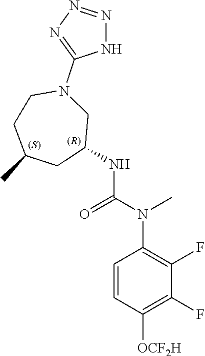 |
| | | 1-(4-(difluoromethoxy)-2,3- |
| | | difluorophenyl)-1-methyl-3- |
| | | ((3R,5S)-5-methyl-1-(1H- |
| | | tetrazol-5-yl)azepan-3-yl)urea |
| TA | 40D* | 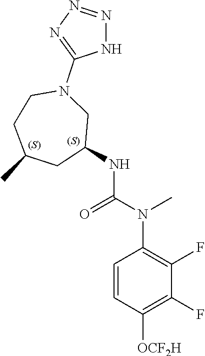 |
| | | 1-(4-(difluoromethoxy)-2,3- |
| | | difluorophenyl)-1-methyl-3- |
| | | ((3S,5S)-5-methyl-1-(1H- |
| | | tetrazol-5-yl)azepan-3-yl)urea |
| TA | 41A* | 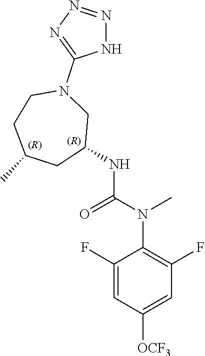 |
| | | 1-(2,6-difluoro-4- |
| | | (trifluoromethoxy)phenyl)-1- |
| | | methyl-3-((3R,5R)-5-methyl-1- |
| | | (1H-tetrazol-5-yl)azepan-3- |
| | | yl)urea |
| TA | 41B* | 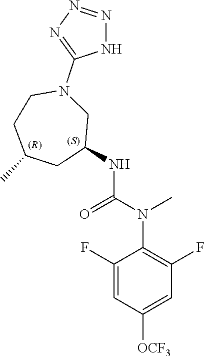 |
| | | 1-(2,6-difluoro-4- |
| | | (trifluoromethoxy)phenyl)-1- |
| | | methyl-3-((3S,5R)-5-methyl-1- |
| | | (1H-tetrazol-5-yl)azepan-3- |
| | | yl)urea |
| TA | 41C* | 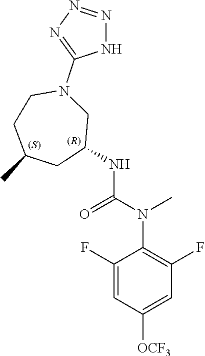 |
| | | 1-(2,6-difluoro-4- |
| | | (trifluoromethoxy)phenyl)-1- |
| | | methyl-3-((3R,5S)-5-methyl-1- |
| | | (1H-tetrazol-5-yl)azepan-3- |
| | | yl)urea |
| TA | Rac-65 | 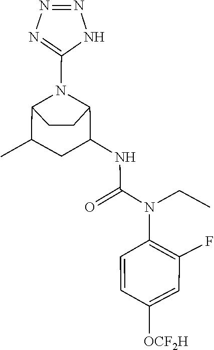 |
| | | 1-(4-(difluoromethoxy)-2- |
| | | fluorophenyl)-1-ethyl-3-(4- |
| | | methyl-8-(1H-tetrazol-5-yl)-8- |
| | | azabicyclo[3.2.1]octan-2- |
| | | yl)urea |
| 5 | 65A* | 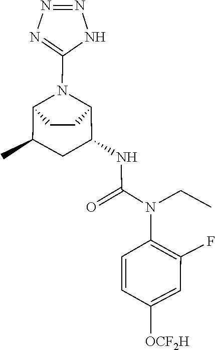 |
| | | 1-(4-(difluoromethoxy)-2- |
| | | fluorophenyl)-1-ethyl-3- |
| | | ((1R,2R,4R,5S)-4-methyl-8- |
| | | (1H-tetrazol-5-yl)-8- |
| | | azabicyclo[3.2.1]octan-2- |
| | | yl)urea |
| 5 | 65D* | 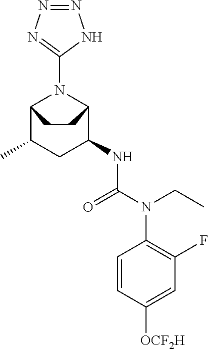 |
| | | 1-(4-(difluoromethoxy)-2- |
| | | fluorophenyl)-1-ethyl-3- |
| | | ((1S,2S,4S,5R)-4-methyl-8- |
| | | (1H-tetrazol-5-yl)-8- |
| | | azabicyclo[3.2.1]octan-2- |
| | | yl)urea |
| TA | 66 | 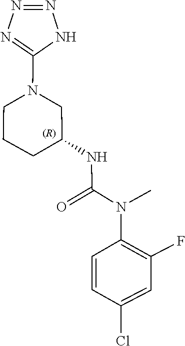 |
| | | (R)-3-(1-(1H-tetrazol-5- |
| | | yl)piperidin-3-yl)-1-(4-chloro- |
| | | 2-fluorophenyl)-1-methylurea |
| 4 | 42′* | 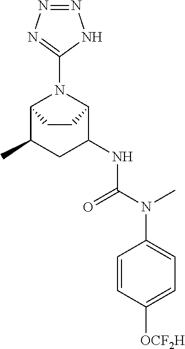 |
| | | 1-(4-(difluoromethoxy)phenyl)-1- |
| | | methyl-3-((1R,4R,5S)-4-methyl-8- |
| | | (1H-tetrazol-5-yl)-8- |
| | | azabicyclo[3.2.1]octan-2-yl)urea |
| 4 | 42″* | 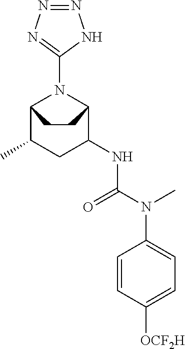 |
| | | 1-(4-(difluoromethoxy)phenyl)-1- |
| | | methyl-3-((1S,4S,5R)-4-methyl-8- |
| | | (1H-tetrazol-5-yl)-8- |
| | | azabicyclo[3.2.1]octan-2-yl)urea |
| 4 | 42A* | 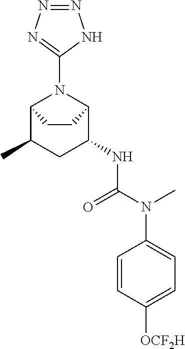 |
| | | 1-(4-(difluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| 4 | 42B* | 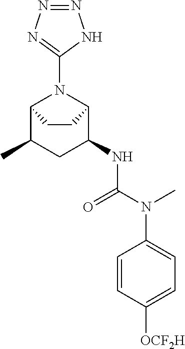 |
| | | 1-(4-(difluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| 4 | 42C* | 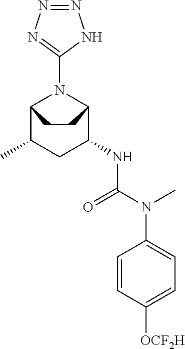 |
| | | 1-(4-(difluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| 4 | 42D* | 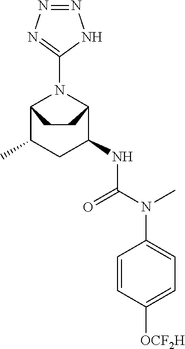 |
| | | 1-(4-(difluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | 43A* | 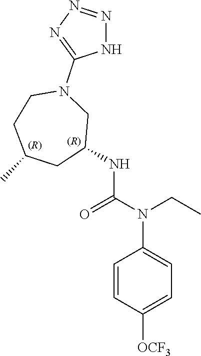 |
| | | 1-ethyl-3-((3R,5R)-5-methyl-1-(1H- |
| | | tetrazol-5-yl)azepan-3-yl)-1-(4- |
| | | (trifluoromethoxy)phenyl)urea |
| TA | 43B* | 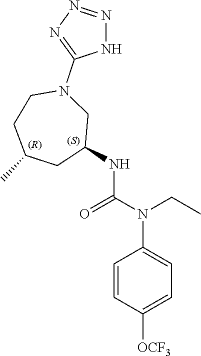 |
| | | 1-ethyl-3-((3S,5R)-5-methyl-1-(1H- |
| | | tetrazol-5-yl)azepan-3-yl)-1-(4- |
| | | (trifluoromethoxy)phenyl)urea |
| TA | 43C* | 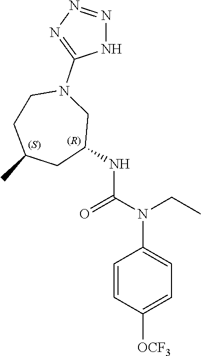 |
| | | 1-ethyl-3-((3R,5S)-5-methyl-1-(1H- |
| | | tetrazol-5-yl)azepan-3-yl)-1-(4- |
| | | (trifluoromethoxy)phenyl)urea |
| TA | 43D* | 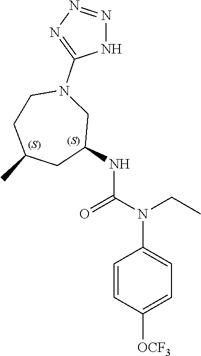 |
| | | 1-ethyl-3-((3S,5S)-5-methyl-1-(1H- |
| | | tetrazol-5-yl)azepan-3-yl)-1-(4- |
| | | (trifluoromethoxy)phenyl)urea |
| TA | 44A* | 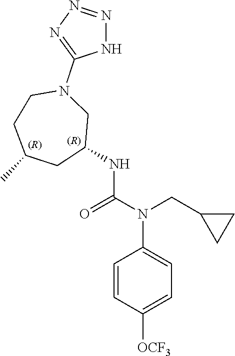 |
| | | 1-(cyclopropylmethyl)-3-((3R,5R)- |
| | | 5-methyl-1-(1H-tetrazol-5- |
| | | yl)azepan-3-yl)-1-(4- |
| | | (trifluoromethoxy)phenyl)urea |
| TA | 44B* | 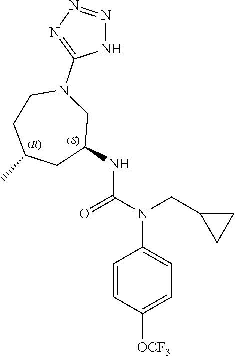 |
| | | 1-(cyclopropylmethyl)-3-((3S,5R)- |
| | | 5-methyl-1-(1H-tetrazol-5- |
| | | yl)azepan-3-yl)-1-(4- |
| | | (trifluoromethoxy)phenyl)urea |
| TA | 44C* | 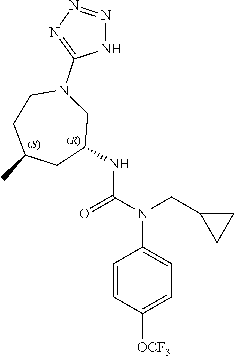 |
| | | 1-(cyclopropylmethyl)-3-((3R,5S)- |
| | | 5-methyl-1-(1H-tetrazol-5- |
| | | yl)azepan-3-yl)-1-(4- |
| | | (trifluoromethoxy)phenyl)urea |
| TA | 44D* | 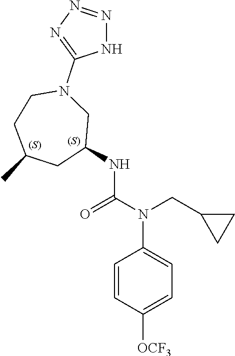 |
| | | 1-(cyclopropylmethyl)-3-((3S,5S)- |
| | | 5-methyl-1-(1H-tetrazol-5- |
| | | yl)azepan-3-yl)-1-(4- |
| | | (trifluoromethoxy)phenyl)urea |
| TA | Rac-45 | 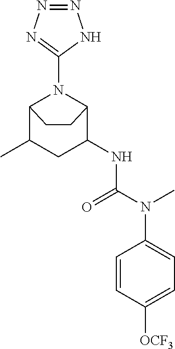 [SMILES: CC1CC(NC(=O)N(C)c2ccc(OC(F)(F)F)cc2)C2CCC1N2c1nnn[nH]1] |
| | | 1-methyl-3-(4-methyl-8-(1H- |
| | | tetrazol-5-yl)-8- |
| | | azabicyclo[3.2.1]octan-2-yl)-1-(4- |
| | | (trifluoromethoxy)phenyl)urea |
| TA | 45′* | 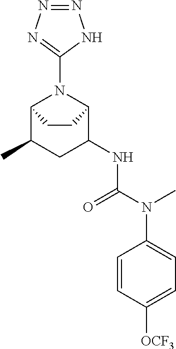 [SMILES: C[C@@H]1CC(NC(=O)N(C)c2ccc(OC(F)(F)F)cc2)[C@H]2CC[C@@H]1N2c1nnn[nH]1] |
| | | 1-methyl-3-((1R,4R,5S)-4-methyl- |
| | | 8-(1H-tetrazol-5-yl)-8- |
| | | azabicyclo[3.2.1]octan-2-yl)-1-(4- |
| | | (trifluoromethoxy)phenyl)urea |
| TA | 45″* | 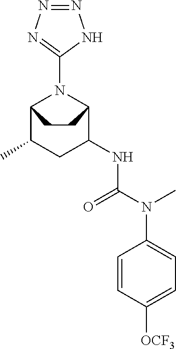 [SMILES: C[C@H]1CC(NC(=O)N(C)c2ccc(OC(F)(F)F)cc2)[C@@H]2CC[C@H]1N2c1nnn[nH]1] |
| | | 1-methyl-3-((1S,4S,5R)-4-methyl- |
| | | 8-(1H-tetrazol-5-yl)-8- |
| | | azabicyclo[3.2.1]octan-2-yl)-1-(4- |
| | | (trifluoromethoxy)phenyl)urea |
| TA | 45A* | 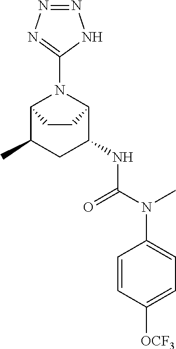 [SMILES: C[C@@H]1C[C@@H](NC(=O)N(C)c2ccc(OC(F)(F)F)cc2)[C@H]2CC[C@@H]1N2c1nnn[nH]1] |
| | | 1-methyl-3-((1R,2R,4R,5S)-4- |
| | | methyl-8-(1H-tetrazol-5-yl)-8- |
| | | azabicyclo[3.2.1]octan-2-yl)-1-(4- |
| | | (trifluoromethoxy)phenyl)urea |
| TA | 45B* | 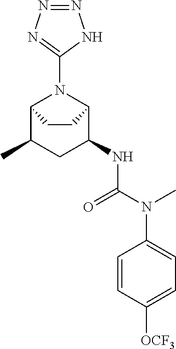 [SMILES: C[C@@H]1C[C@H](NC(=O)N(C)c2ccc(OC(F)(F)F)cc2)[C@H]2CC[C@@H]1N2c1nnn[nH]1] |
| | | 1-methyl-3-((1R,2S,4R,5S)-4- |
| | | methyl-8-(1H-tetrazol-5-yl)-8- |
| | | azabicyclo[3.2.1]octan-2-yl)-1-(4- |
| | | (trifluoromethoxy)phenyl)urea |
| TA | 45C* | 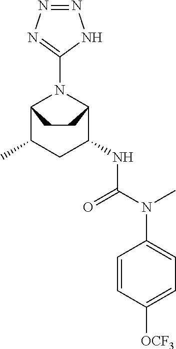 [SMILES: C[C@H]1C[C@@H](NC(=O)N(C)c2ccc(OC(F)(F)F)cc2)[C@@H]2CC[C@H]1N2c1nnn[nH]1] |
| | | 1-methyl-3-((1S,2R,4S,5R)-4- |
| | | methyl-8-(1H-tetrazol-5-yl)-8- |
| | | azabicyclo[3.2.1]octan-2-yl)-1-(4- |
| | | (trifluoromethoxy)phenyl)urea |
| TA | 45D* | 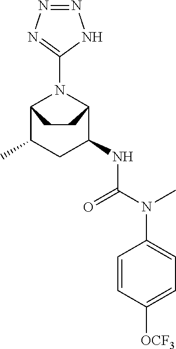 [SMILES: C[C@H]1C[C@H](NC(=O)N(C)c2ccc(OC(F)(F)F)cc2)[C@@H]2CC[C@H]1N2c1nnn[nH]1] |
| | | 1-methyl-3-((1S,2S,4S,5R)-4- |
| | | methyl-8-(1H-tetrazol-5-yl)-8- |
| | | azabicyclo[3.2.1]octan-2-yl)-1-(4- |
| | | (trifluoromethoxy)phenyl)urea |
| TA | Rac-46 | 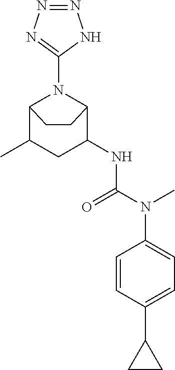 |
| | | 1-(4-cyclopropylphenyl)-1-methyl- |
| | | 3-(4-methyl-8-(1H-tetrazol-5-yl)-8- |
| | | azabicyclo[3.2.1]octan-2-yl)urea |
| TA | 46′* | 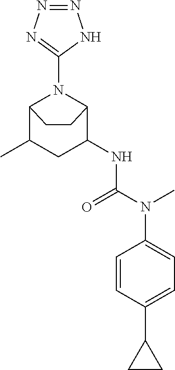 |
| | | 1-(4-cyclopropylphenyl)-1-methyl- |
| | | 3-((1R,4R,5S)-4-methyl-8-(1H- |
| | | tetrazol-5-yl)-8- |
| | | azabicyclo[3.2.1]octan-2-yl)urea |
| TA | 46″* | 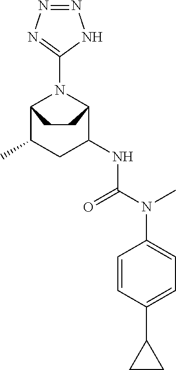 [SMILES: C[C@H]1CC(NC(=O)N(C)c2ccc(C3CC3)cc2)[C@@H]2CC[C@H]1N2c1nnn[nH]1] |
| | | 1-(4-cyclopropylphenyl)-1-methyl- |
| | | 3-((1S,4S,5R)-4-methyl-8-(1H- |
| | | tetrazol-5-yl)-8- |
| | | azabicyclo[3.2.1]octan-2-yl)urea |
| TA | 46A* | 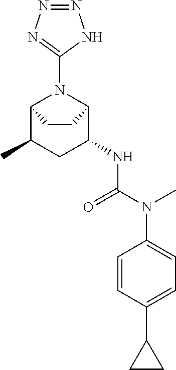 |
| | | 1-(4-cyclopropylphenyl)-1-methyl- |
| | | 3-((1R,2R,4R,5S)-4-methyl-8-(1H- |
| | | tetrazol-5-yl)-8- |
| | | azabicyclo[3.2.1]octan-2-yl)urea |
| TA | 46B* | 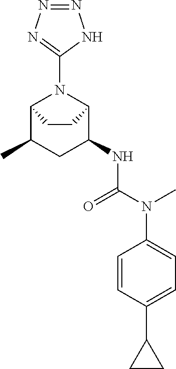 |
| | | 1-(4-cyclopropylphenyl)-1-methyl- |
| | | 3-((1R,2S,4R,5S)-4-methyl-8-(1H- |
| | | tetrazol-5-yl)-8- |
| | | azabicyclo[3.2.1]octan-2-yl)urea |
| TA | 46C* | 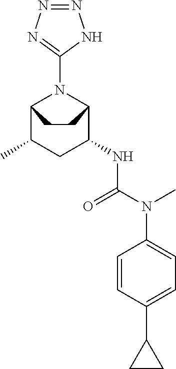 |
| | | 1-(4-cyclopropylphenyl)-1-methyl- |
| | | 3-((1S,2R,4S,5R)-4-methyl-8-(1H- |
| | | tetrazol-5-yl)-8- |
| | | azabicyclo[3.2.1]octan-2-yl)urea |
| TA | 46D* | 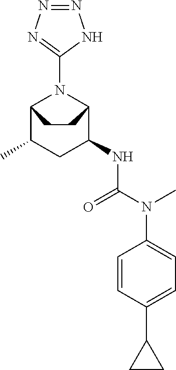 [SMILES: C[C@H]1C[C@H](NC(=O)N(C)c2ccc(C3CC3)cc2)[C@@H]2CC[C@H]1N2c1nnn[nH]1] |
| | | 1-(4-cyclopropylphenyl)-1-methyl- |
| | | 3-((1S,2S,4S,5R)-4-methyl-8-(1H- |
| | | tetrazol-5-yl)-8- |
| | | azabicyclo[3.2.1]octan-2-yl)urea |
| TA | RAc-47 | 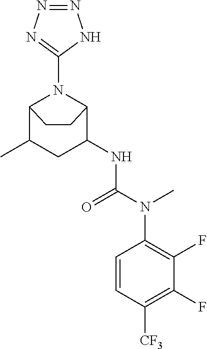 [SMILES: CC1CC(NC(=O)N(C)c2ccc(C(F)(F)F)c(F)c2F)C2CCC1N2c1nnn[nH]1] |
| | | 1-(2,3-difluoro-4- |
| | | (trifluoromethyl)phenyl)-1-methyl- |
| | | 3-(4-methyl-8-(1H-tetrazol-5-yl)-8- |
| | | azabicyclo[3.2.1]octan-2-yl)urea |
| TA | 47′* | 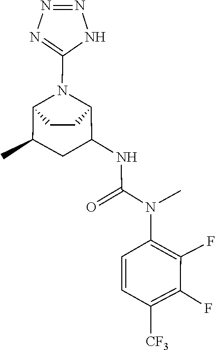 [SMILES: C[C@@H]1CC(NC(=O)N(C)c2ccc(C(F)(F)F)c(F)c2F)[C@H]2CC[C@@H]1N2c1nnn[nH]1] |
| | | 1-(2,3-difluoro-4- |
| | | (trifluoromethyl)phenyl)-1-methyl- |
| | | 3-((1R,2R,5S)-4-methyl-8-(1H- |
| | | tetrazol-5-yl)-8- |
| | | azabicyclo[3.2.1]octan-2-yl)urea |
| TA | 47″* | 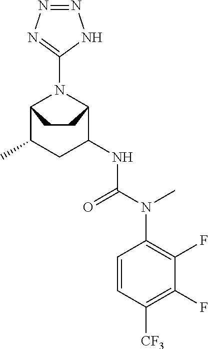 [SMILES: C[C@H]1CC(NC(=O)N(C)c2ccc(C(F)(F)F)c(F)c2F)[C@@H]2CC[C@H]1N2c1nnn[nH]1] |
| | | 1-(2,3-difluoro-4- |
| | | (trifluoromethyl)phenyl)-1-methyl- |
| | | 3-((1S,2S,5R)-4-methyl-8-(1H- |
| | | tetrazol-5-yl)-8- |
| | | azabicyclo[3.2.1]octan-2-yl)urea |
| TA | 47A* | 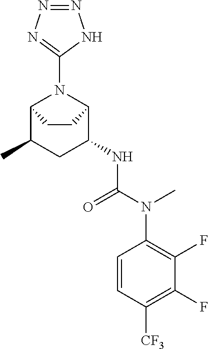 [SMILES: C[C@@H]1C[C@@H](NC(=O)N(C)c2ccc(C(F)(F)F)c(F)c2F)[C@H]2CC[C@@H]1N2c1nnn[nH]1] |
| | | 1-(2,3-difluoro-4- |
| | | (trifluoromethyl)phenyl)-1-methyl- |
| | | 3-((1R,2R,4R,5S)-4-methyl-8-(1H- |
| | | tetrazol-5-yl)-8- |
| | | azabicyclo[3.2.1]octan-2-yl)urea |
| TA | 47B* | 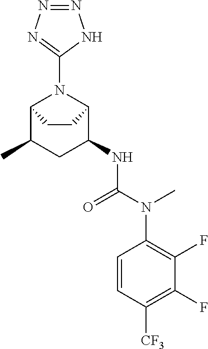 [SMILES: C[C@@H]1C[C@H](NC(=O)N(C)c2ccc(C(F)(F)F)c(F)c2F)[C@H]2CC[C@@H]1N2c1nnn[nH]1] |
| | | 1-(2,3-difluoro-4- |
| | | (trifluoromethyl)phenyl)-1-methyl- |
| | | 3-((1R,2S,4R,5S)-4-methyl-8-(1H- |
| | | tetrazol-5-yl)-8- |
| | | azabicyclo[3.2.1]octan-2-yl)urea |
| TA | 47C* | 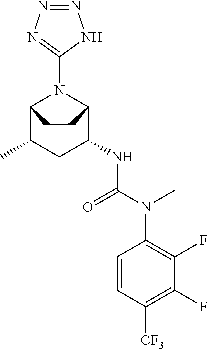 [SMILES: C[C@H]1C[C@@H](NC(=O)N(C)c2ccc(C(F)(F)F)c(F)c2F)[C@@H]2CC[C@H]1N2c1nnn[nH]1] |
| | | 1-(2,3-difluoro-4- |
| | | (trifluoromethyl)phenyl)-1-methyl- |
| | | 3-((1S,2R,4S,5R)-4-methyl-8-(1H- |
| | | tetrazol-5-yl)-8- |
| | | azabicyclo[3.2.1]octan-2-yl)urea |
| TA | 47D* | 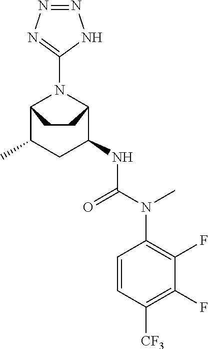 [SMILES: C[C@H]1C[C@H](NC(=O)N(C)c2ccc(C(F)(F)F)c(F)c2F)[C@@H]2CC[C@H]1N2c1nnn[nH]1] |
| | | 1-(2,3-difluoro-4- |
| | | (trifluoromethyl)phenyl)-1-methyl- |
| | | 3-((1S,2S,4S,5R)-4-methyl-8-(1H- |
| | | tetrazol-5-yl)-8- |
| | | azabicyclo[3.2.1]octan-2-yl)urea |
| TA | Rac-48 | 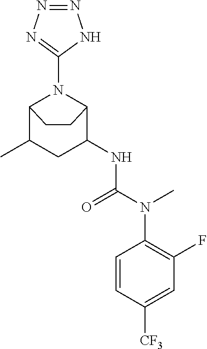 [SMILES: CC1CC(NC(=O)N(C)c2ccc(C(F)(F)F)cc2F)C2CCC1N2c1nnn[nH]1] |
| | | 1-(2-fluoro-4- |
| | | (trifluoromethyl)phenyl)-1-methyl- |
| | | 3-(4-methyl-8-(1H-tetrazol-5-yl)-8- |
| | | azabicyclo[3.2.1]octan-2-yl)urea |
| TA | 48′* | 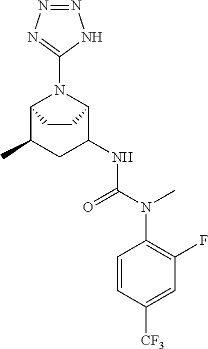 [SMILES: C[C@@H]1CC(NC(=O)N(C)c2ccc(C(F)(F)F)cc2F)[C@H]2CC[C@@H]1N2c1nnn[nH]1] |
| | | 1-(2-fluoro-4- |
| | | (trifluoromethyl)phenyl)-1-methyl- |
| | | 3-((1R,2R,5S)-4-methyl-8-(1H- |
| | | tetrazol-5-yl)-8- |
| | | azabicyclo[3.2.1]octan-2-yl)urea |
| TA | 48″* | 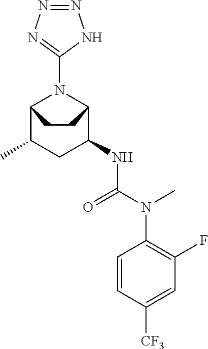 [SMILES: C[C@H]1C[C@H](NC(=O)N(C)c2ccc(C(F)(F)F)cc2F)[C@@H]2CC[C@H]1N2c1nnn[nH]1] |
| | | 1-(2-fluoro-4- |
| | | (trifluoromethyl)phenyl)-1-methyl- |
| | | 3-((1S,2S,5R)-4-methyl-8-(1H- |
| | | tetrazol-5-yl)-8- |
| | | azabicyclo[3.2.1]octan-2-yl)urea |
| TA | 48A* | 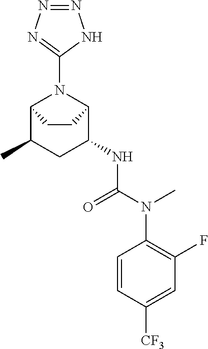 [SMILES: C[C@@H]1C[C@@H](NC(=O)N(C)c2ccc(C(F)(F)F)cc2F)[C@H]2CC[C@@H]1N2c1nnn[nH]1] |
| | | 1-(2-fluoro-4- |
| | | (trifluoromethyl)phenyl)-1-methyl- |
| | | 3-((1R,2R,4R,5S)-4-methyl-8-(1H- |
| | | tetrazol-5-yl)-8- |
| | | azabicyclo[3.2.1]octan-2-yl)urea |
| TA | 48B* | 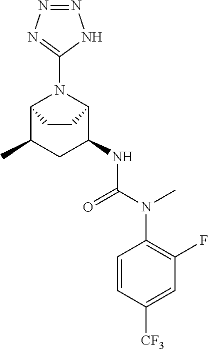 [SMILES: C[C@@H]1C[C@H](NC(=O)N(C)c2ccc(C(F)(F)F)cc2F)[C@H]2CC[C@@H]1N2c1nnn[nH]1] |
| | | 1-(2-fluoro-4- |
| | | (trifluoromethyl)phenyl)-1-methyl- |
| | | 3-((1R,2S,4R,5S)-4-methyl-8-(1H- |
| | | tetrazol-5-yl)-8- |
| | | azabicyclo[3.2.1]octan-2-yl)urea |
| TA | 48C* | 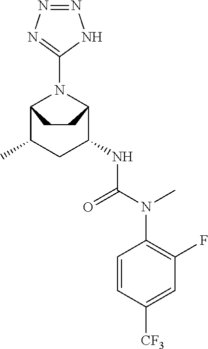 [SMILES: C[C@H]1C[C@@H](NC(=O)N(C)c2ccc(C(F)(F)F)cc2F)[C@@H]2CC[C@H]1N2c1nnn[nH]1] |
| | | 1-(2-fluoro-4- |
| | | (trifluoromethyl)phenyl)-1-methyl- |
| | | 3-((1S,2R,4S,5R)-4-methyl-8-(1H- |
| | | tetrazol-5-yl)-8- |
| | | azabicyclo[3.2.1]octan-2-yl)urea |
| TA | 48D* | 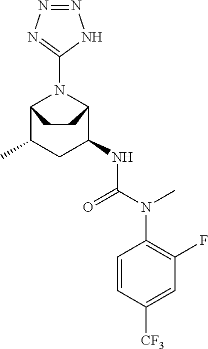 [SMILES: C[C@H]1C[C@H](NC(=O)N(C)c2ccc(C(F)(F)F)cc2F)[C@@H]2CC[C@H]1N2c1nnn[nH]1] |
| | | 1-(2-fluoro-4- |
| | | (trifluoromethyl)phenyl)-1-methyl- |
| | | 3-((1S,2S,4S,5R)-4-methyl-8-(1H- |
| | | tetrazol-5-yl)-8- |
| | | azabicyclo[3.2.1]octan-2-yl)urea |
| TA | Rac-49 | 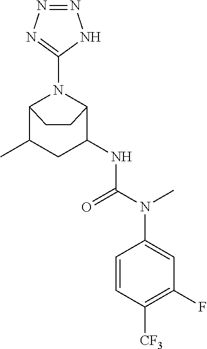 [SMILES: CC1CC(NC(=O)N(C)c2ccc(C(F)(F)F)c(F)c2)C2CCC1N2c1nnn[nH]1] |
| | | 1-(3-fluoro-4- |
| | | (trifluoromethyl)phenyl)-1-methyl- |
| | | 3-(4-methyl-8-(1H-tetrazol-5-yl)-8- |
| | | azabicyclo[3.2.1]octan-2-yl)urea |
| TA | 49′* | 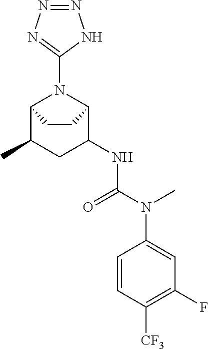 [SMILES: C[C@@H]1CC(NC(=O)N(C)c2ccc(C(F)(F)F)c(F)c2)[C@H]2CC[C@@H]1N2c1nnn[nH]1] |
| | | 1-(3-fluoro-4- |
| | | (trifluoromethyl)phenyl)-1-methyl- |
| | | 3-((1R,2R,5S)-4-methyl-8-(1H- |
| | | tetrazol-5-yl)-8- |
| | | azabicyclo[3.2.1]octan-2-yl)urea |
| TA | 49″* | 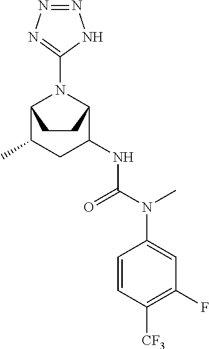 [SMILES: C[C@H]1CC(NC(=O)N(C)c2ccc(C(F)(F)F)c(F)c2)[C@@H]2CC[C@H]1N2c1nnn[nH]1] |
| | | 1-(3-fluoro-4- |
| | | (trifluoromethyl)phenyl)-1-methyl- |
| | | 3-((1S,2S,5R)-4-methyl-8-(1H- |
| | | tetrazol-5-yl)-8- |
| | | azabicyclo[3.2.1]octan-2-yl)urea |
| TA | 49A* | 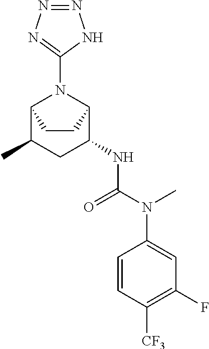 [SMILES: C[C@@H]1C[C@@H](NC(=O)N(C)c2ccc(C(F)(F)F)c(F)c2)[C@H]2CC[C@@H]1N2c1nnn[nH]1] |
| | | 1-(3-fluoro-4- |
| | | (trifluoromethyl)phenyl)-1-methyl- |
| | | 3-((1R,2R,4R,5S)-4-methyl-8-(1H- |
| | | tetrazol-5-yl)-8- |
| | | azabicyclo[3.2.1]octan-2-yl)urea |
| TA | 49B* | 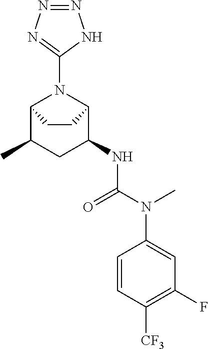 [SMILES: C[C@@H]1C[C@H](NC(=O)N(C)c2ccc(C(F)(F)F)c(F)c2)[C@H]2CC[C@@H]1N2c1nnn[nH]1] |
| | | 1-(3-fluoro-4- |
| | | (trifluoromethyl)phenyl)-1-methyl- |
| | | 3-((1R,2S,4R,5S)-4-methyl-8-(1H- |
| | | tetrazol-5-yl)-8- |
| | | azabicyclo[3.2.1]octan-2-yl)urea |
| TA | 49C* | 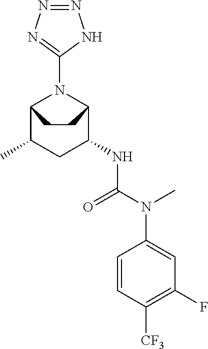 [SMILES: C[C@H]1C[C@@H](NC(=O)N(C)c2ccc(C(F)(F)F)c(F)c2)[C@@H]2CC[C@H]1N2c1nnn[nH]1] |
| | | 1-(3-fluoro-4- |
| | | (trifluoromethyl)phenyl)-1-methyl- |
| | | 3-((1S,2R,4S,5R)-4-methyl-8-(1H- |
| | | tetrazol-5-yl)-8- |
| | | azabicyclo[3.2.1]octan-2-yl)urea |
| TA | 49D* | 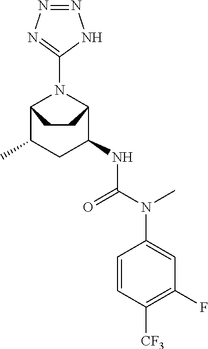 [SMILES: C[C@H]1C[C@H](NC(=O)N(C)c2ccc(C(F)(F)F)c(F)c2)[C@@H]2CC[C@H]1N2c1nnn[nH]1] |
| | | 1-(3-fluoro-4- |
| | | (trifluoromethyl)phenyl)-1-methyl- |
| | | 3-((1S,2S,4S,5R)-4-methyl-8-(1H- |
| | | tetrazol-5-yl)-8- |
| | | azabicyclo[3.2.1]octan-2-yl)urea |
| TA | Rac-50 | 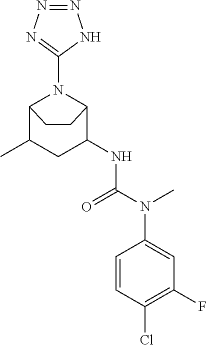 [SMILES: CC1CC(NC(=O)N(C)c2ccc(Cl)c(F)c2)C2CCC1N2c1nnn[nH]1] |
| | | 1-(4-chloro-3-fluorophenyl)-1- |
| | | methyl-3-(4-methyl-8-(1H-tetrazol- |
| | | 5-yl)-8-azabicyclo[3.2.1]octan-2- |
| | | yl)urea |
| TA | 50′* | 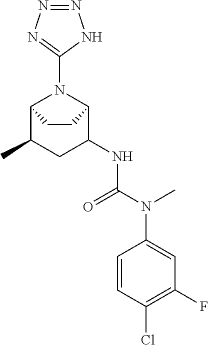 [SMILES: C[C@@H]1CC(NC(=O)N(C)c2ccc(Cl)c(F)c2)[C@H]2CC[C@@H]1N2c1nnn[nH]1] |
| | | 1-(4-chloro-3-fluorophenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 50″* | 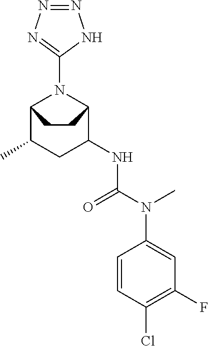 [SMILES: C[C@H]1CC(NC(=O)N(C)c2ccc(Cl)c(F)c2)[C@@H]2CC[C@H]1N2c1nnn[nH]1] |
| | | 1-(4-chloro-3-fluorophenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 50A* | 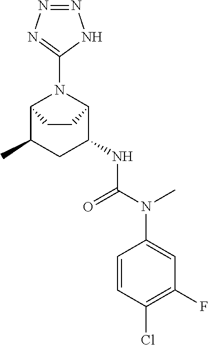 [SMILES: C[C@@H]1C[C@@H](NC(=O)N(C)c2ccc(Cl)c(F)c2)[C@H]2CC[C@@H]1N2c1nnn[nH]1] |
| | | 1-(4-chloro-3-fluorophenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 50B* | 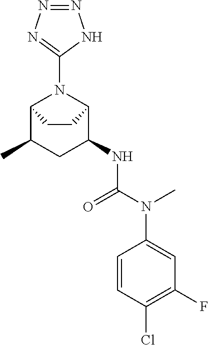 [SMILES: C[C@@H]1C[C@H](NC(=O)N(C)c2ccc(Cl)c(F)c2)[C@H]2CC[C@@H]1N2c1nnn[nH]1] |
| | | 1-(4-chloro-3-fluorophenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 50C* | 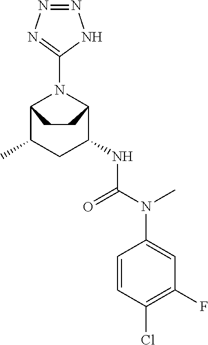 [SMILES: C[C@H]1C[C@@H](NC(=O)N(C)c2ccc(Cl)c(F)c2)[C@@H]2CC[C@H]1N2c1nnn[nH]1] |
| | | 1-(4-chloro-3-fluorophenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 50D* | 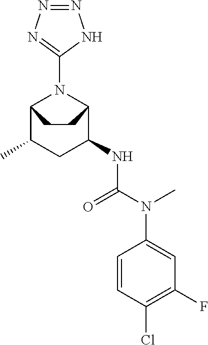 [SMILES: C[C@H]1C[C@H](NC(=O)N(C)c2ccc(Cl)c(F)c2)[C@@H]2CC[C@H]1N2c1nnn[nH]1] |
| | | 1-(4-chloro-3-fluorophenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | Rac-51 | 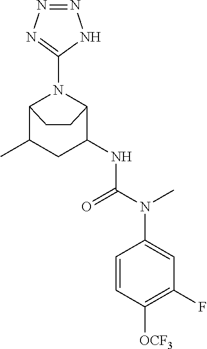 [SMILES: CC1CC(NC(=O)N(C)c2ccc(OC(F)(F)F)c(F)c2)C2CCC1N2c1nnn[nH]1] |
| | | 1-(3-fluoro-4- |
| | | (trifluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 51′* | 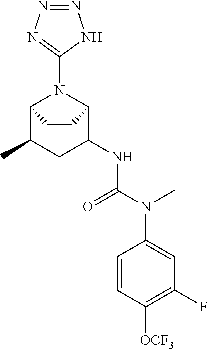 [SMILES: C[C@@H]1CC(NC(=O)N(C)c2ccc(OC(F)(F)F)c(F)c2)[C@H]2CC[C@@H]1N2c1nnn[nH]1] |
| | | 1-(3-fluoro-4- |
| | | (trifluoromethoxy)phenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 51″* | 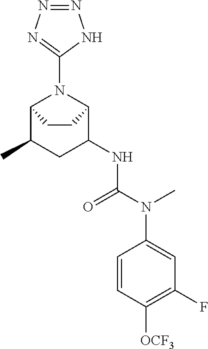 [SMILES: C[C@@H]1CC(NC(=O)N(C)c2ccc(OC(F)(F)F)c(F)c2)[C@H]2CC[C@@H]1N2c1nnn[nH]1] |
| | | 1-(3-fluoro-4- |
| | | (trifluoromethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 51A* | 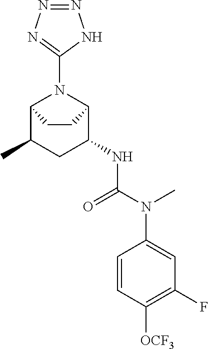 [SMILES: C[C@@H]1C[C@@H](NC(=O)N(C)c2ccc(OC(F)(F)F)c(F)c2)[C@H]2CC[C@@H]1N2c1nnn[nH]1] |
| | | 1-(3-fluoro-4- |
| | | (trifluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 51B* | 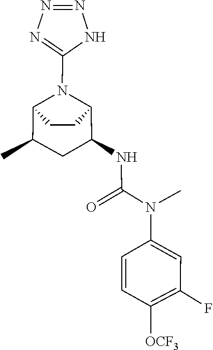 [SMILES: C[C@@H]1C[C@H](NC(=O)N(C)c2ccc(OC(F)(F)F)c(F)c2)[C@H]2CC[C@@H]1N2c1nnn[nH]1] |
| | | 1-(3-fluoro-4- |
| | | (trifluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 51C* | 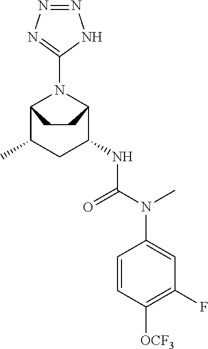 [SMILES: C[C@H]1C[C@@H](NC(=O)N(C)c2ccc(OC(F)(F)F)c(F)c2)[C@@H]2CC[C@H]1N2c1nnn[nH]1] |
| | | 1-(3-fluoro-4- |
| | | (trifluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 51D* | 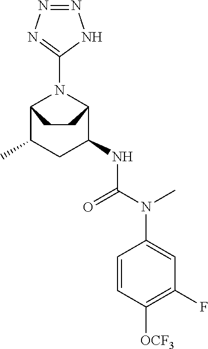 [SMILES: C[C@H]1C[C@H](NC(=O)N(C)c2ccc(OC(F)(F)F)c(F)c2)[C@@H]2CC[C@H]1N2c1nnn[nH]1] |
| | | 1-(3-fluoro-4- |
| | | (trifluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | Rac-52 | 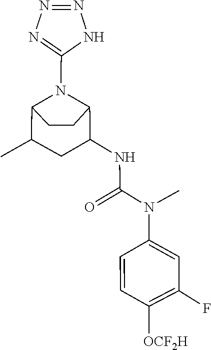 |
| | | 1-(4-(difluoromethoxy)-3- |
| | | fluorophenyl)-1-methyl-3-(4- |
| | | methyl-8-(1H-tetrazol-5-yl)-8- |
| | | azabicyclo[3.2.1]octan-2-yl)urea |
| TA | 52′* | 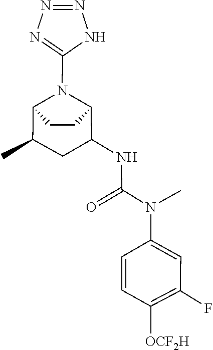 |
| | | 1-(4-(difluoromethoxy)-3- |
| | | fluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 52″* | 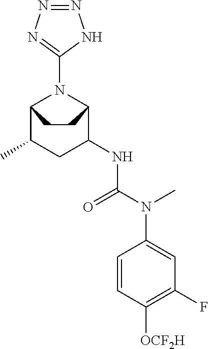 |
| | | 1-(4-(difluoromethoxy)-3- |
| | | fluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 52A* | 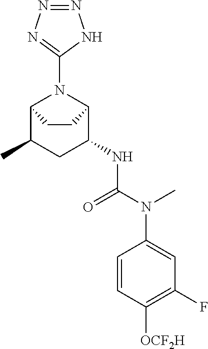 |
| | | 1-(4-(difluoromethoxy)-3- |
| | | fluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 52B* | 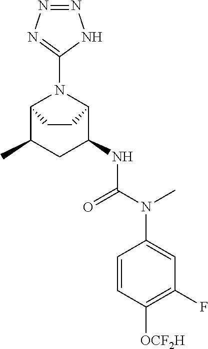 |
| | | 1-(4-(difluoromethoxy)-3- |
| | | fluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 52C* | 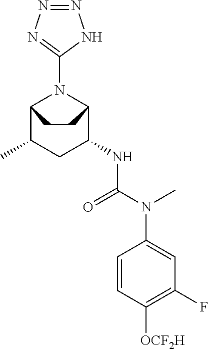 |
| | | 1-(4-(difluoromethoxy)-3- |
| | | fluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 52D* | 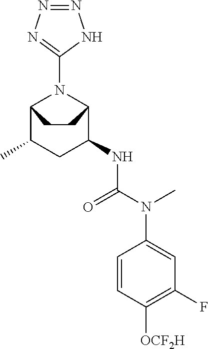 |
| | | 1-(4-(difluoromethoxy)-3- |
| | | fluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-53 | 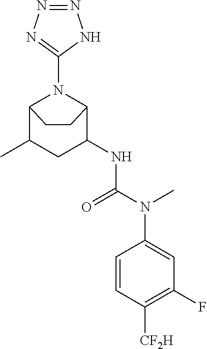 [SMILES: CC1CC(NC(=O)N(C)c2ccc(C(F)F)c(F)c2)C2CCC1N2c1nnn[nH]1] |
| | | 1-(4-(difluoromethyl)-3- |
| | | fluorophenyl)-1-methyl-3-(4- |
| | | methyl-8-(1H-tetrazol-5-yl)-8- |
| | | azabicyclo[3.2.1]octan-2-yl)urea |
| TA | 53′* | 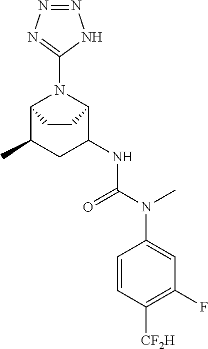 [SMILES: C[C@@H]1CC(NC(=O)N(C)c2ccc(C(F)F)c(F)c2)[C@H]2CC[C@@H]1N2c1nnn[nH]1] |
| | | 1-(4-(difluoromethyl)-3- |
| | | fluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 53″* | 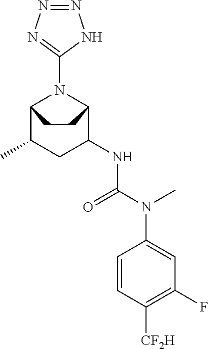 [SMILES: C[C@H]1CC(NC(=O)N(C)c2ccc(C(F)F)c(F)c2)[C@@H]2CC[C@H]1N2c1nnn[nH]1] |
| | | 1-(4-(difluoromethyl)-3- |
| | | fluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 53A* | 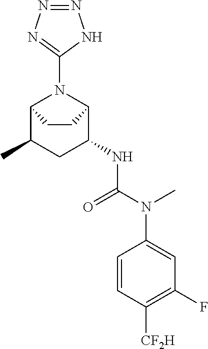 [SMILES: C[C@@H]1C[C@@H](NC(=O)N(C)c2ccc(C(F)F)c(F)c2)[C@H]2CC[C@@H]1N2c1nnn[nH]1] |
| | | 1-(4-(difluoromethyl)-3- |
| | | fluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 53B* | 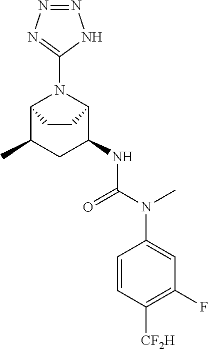 [SMILES: C[C@@H]1C[C@H](NC(=O)N(C)c2ccc(C(F)F)c(F)c2)[C@H]2CC[C@@H]1N2c1nnn[nH]1] |
| | | 1-(4-(difluoromethyl)-3- |
| | | fluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 53C* | 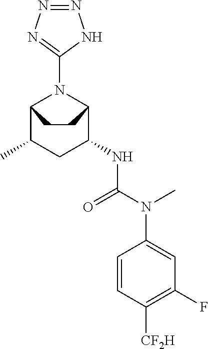 [SMILES: C[C@H]1C[C@@H](NC(=O)N(C)c2ccc(C(F)F)c(F)c2)[C@@H]2CC[C@H]1N2c1nnn[nH]1] |
| | | 1-(4-(difluoromethyl)-3- |
| | | fluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 53D* | 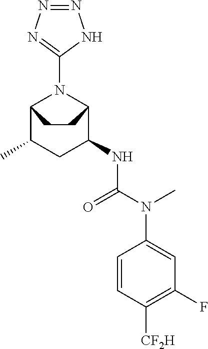 [SMILES: C[C@H]1C[C@H](NC(=O)N(C)c2ccc(C(F)F)c(F)c2)[C@@H]2CC[C@H]1N2c1nnn[nH]1] |
| | | 1-(4-(difluoromethyl)-3- |
| | | fluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-54 | 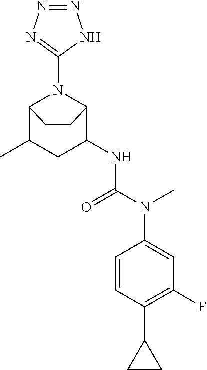 [SMILES: CC1CC(NC(=O)N(C)c2ccc(C3CC3)c(F)c2)C2CCC1N2c1nnn[nH]1] |
| | | 1-(4-cyclopropyl-3-fluorophenyl)-1- |
| | | methyl-3-(4-methyl-8-(1H-tetrazol- |
| | | 5-yl)-8-azabicyclo[3.2.1]octan-2- |
| | | yl)urea |
| TA | 54′* | 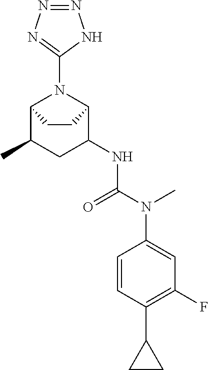 [SMILES: C[C@@H]1CC(NC(=O)N(C)c2ccc(C3CC3)c(F)c2)[C@H]2CC[C@@H]1N2c1nnn[nH]1] |
| | | 1-(4-cyclopropyl-3-fluorophenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 54″* | 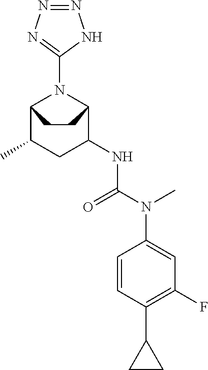 [SMILES: C[C@H]1CC(NC(=O)N(C)c2ccc(C3CC3)c(F)c2)[C@@H]2CC[C@H]1N2c1nnn[nH]1] |
| | | 1-(4-cyclopropyl-3-fluorophenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 54A* | 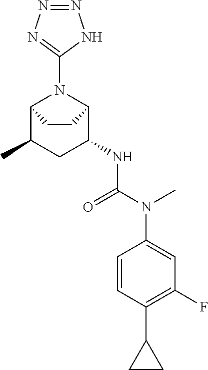 [SMILES: C[C@@H]1C[C@@H](NC(=O)N(C)c2ccc(C3CC3)c(F)c2)[C@H]2CC[C@@H]1N2c1nnn[nH]1] |
| | | 1-(4-cyclopropyl-3-fluorophenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 54B* | 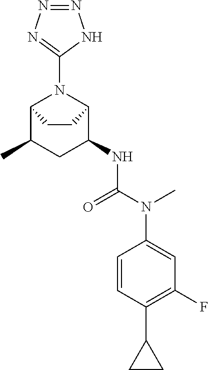 [SMILES: C[C@@H]1C[C@H](NC(=O)N(C)c2ccc(C3CC3)c(F)c2)[C@H]2CC[C@@H]1N2c1nnn[nH]1] |
| | | 1-(4-cyclopropyl-3-fluorophenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 54C* | 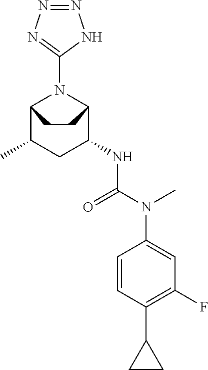 [SMILES: C[C@H]1C[C@@H](NC(=O)N(C)c2ccc(C3CC3)c(F)c2)[C@@H]2CC[C@H]1N2c1nnn[nH]1] |
| | | 1-(4-cyclopropyl-3-fluorophenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 54D* | 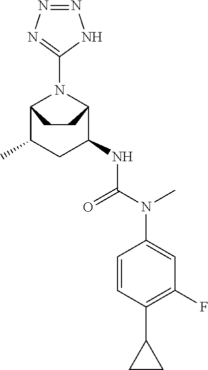 [SMILES: C[C@H]1C[C@H](NC(=O)N(C)c2ccc(C3CC3)c(F)c2)[C@@H]2CC[C@H]1N2c1nnn[nH]1] |
| | | 1-(4-cyclopropyl-3-fluorophenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | Rac-55 | 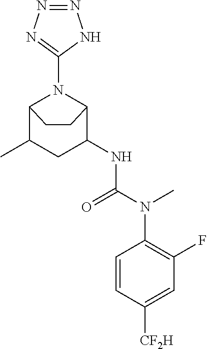 [SMILES: CC1CC(NC(=O)N(C)c2ccc(C(F)F)cc2F)C2CCC1N2c1nnn[nH]1] |
| | | 1-(4-(difluoromethyl)-2- |
| | | fluorophenyl)-1-methyl-3-(4- |
| | | methyl-8-(1H-tetrazol-5-yl)-8- |
| | | azabicyclo[3.2.1]octan-2-yl)urea |
| TA | 55′* | 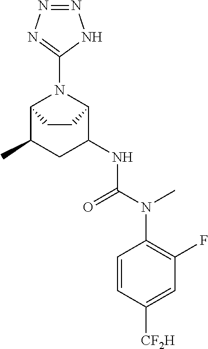 [SMILES: C[C@@H]1CC(NC(=O)N(C)c2ccc(C(F)F)cc2F)[C@H]2CC[C@@H]1N2c1nnn[nH]1] |
| | | 1-(4-(difluoromethyl)-2- |
| | | fluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 55″* | 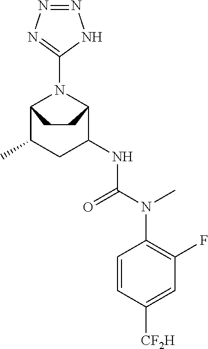 [SMILES: C[C@H]1CC(NC(=O)N(C)c2ccc(C(F)F)cc2F)[C@@H]2CC[C@H]1N2c1nnn[nH]1] |
| | | 1-(4-(difluoromethyl)-2- |
| | | fluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 55A* | 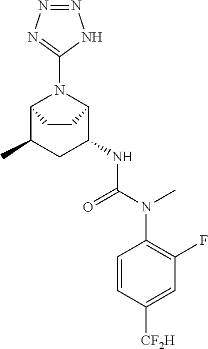 [SMILES: C[C@@H]1C[C@@H](NC(=O)N(C)c2ccc(C(F)F)cc2F)[C@H]2CC[C@@H]1N2c1nnn[nH]1] |
| | | 1-(4-(difluoromethyl)-2- |
| | | fluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 55B* | 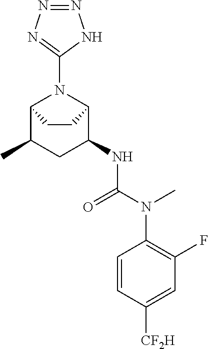 [SMILES: C[C@@H]1C[C@H](NC(=O)N(C)c2ccc(C(F)F)cc2F)[C@H]2CC[C@@H]1N2c1nnn[nH]1] |
| | | 1-(4-(difluoromethyl)-2- |
| | | fluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 55C* | 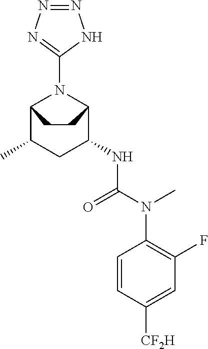 [SMILES: C[C@H]1C[C@@H](NC(=O)N(C)c2ccc(C(F)F)cc2F)[C@@H]2CC[C@H]1N2c1nnn[nH]1] |
| | | 1-(4-(difluoromethyl)-2- |
| | | fluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 55D* | 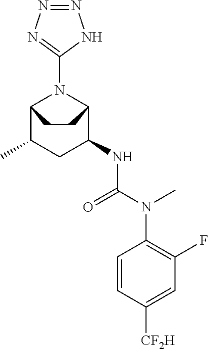 [SMILES: C[C@H]1C[C@H](NC(=O)N(C)c2ccc(C(F)F)cc2F)[C@@H]2CC[C@H]1N2c1nnn[nH]1] |
| | | 1-(4-(difluoromethyl)-2- |
| | | fluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-56 | 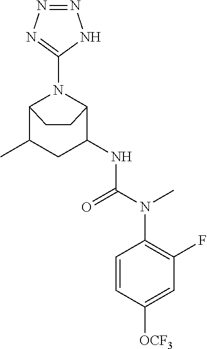 [SMILES: CC1CC(NC(=O)N(C)c2ccc(OC(F)(F)F)cc2F)C2CCC1N2c1nnn[nH]1] |
| | | 1-(2-fluoro-4- |
| | | (trifluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 56′* | 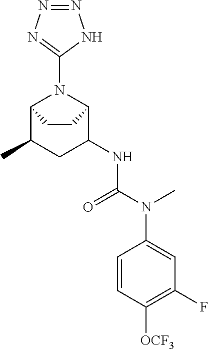 [SMILES: C[C@@H]1CC(NC(=O)N(C)c2ccc(OC(F)(F)F)c(F)c2)[C@H]2CC[C@@H]1N2c1nnn[nH]1] |
| | | 1-(2-fluoro-4- |
| | | (trifluoromethoxy)phenyl)-1- |
| | | methyl-3-((1R,2R,5S)-4-methyl-8- |
| | | (1H-tetrazol-5-yl)-8- |
| | | azabicyclo[3.2.1]octan-2-y)urea |
| TA | 56″* | 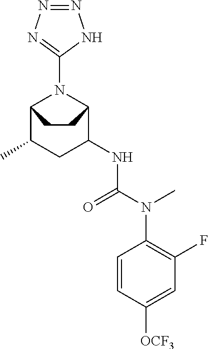 [SMILES: C[C@H]1CC(NC(=O)N(C)c2ccc(OC(F)(F)F)cc2F)[C@@H]2CC[C@H]1N2c1nnn[nH]1] |
| | | 1-(2-fluoro-4- |
| | | (trifluoromethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| 6TA | 56A* | 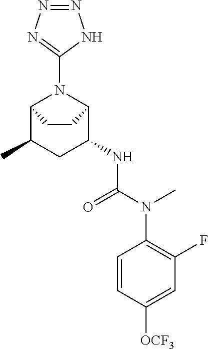 [SMILES: C[C@@H]1C[C@@H](NC(=O)N(C)c2ccc(OC(F)(F)F)cc2F)[C@H]2CC[C@@H]1N2c1nnn[nH]1] |
| | | 1-(2-fluoro-4- |
| | | (trifluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 56B* | 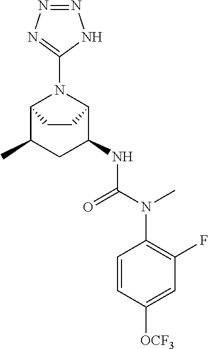 [SMILES: C[C@@H]1C[C@H](NC(=O)N(C)c2ccc(OC(F)(F)F)cc2F)[C@H]2CC[C@@H]1N2c1nnn[nH]1] |
| | | 1-(2-fluoro-4- |
| | | (trifluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| 5 | 56C* | 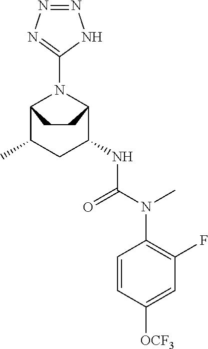 [SMILES: C[C@H]1C[C@@H](NC(=O)N(C)c2ccc(OC(F)(F)F)cc2F)[C@@H]2CC[C@H]1N2c1nnn[nH]1] |
| | | 1-(2-fluoro-4- |
| | | (trifluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| 6TA | 56D* | 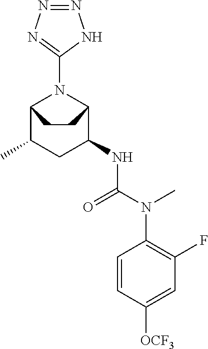 [SMILES: C[C@H]1C[C@H](NC(=O)N(C)c2ccc(OC(F)(F)F)cc2F)[C@@H]2CC[C@H]1N2c1nnn[nH]1] |
| | | 1-(2-fluoro-4- |
| | | (trifluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | Rac-57 | 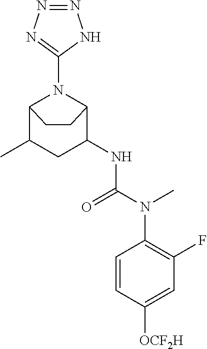 |
| | | 1-(4-(difluoromethoxy)-2- |
| | | fluorophenyl)-1-methyl-3-(4- |
| | | methyl-8-(1H-tetrazol-5-yl)-8- |
| | | azabicyclo[3.2.1]octan-2-yl)urea |
| TA | 57′* | 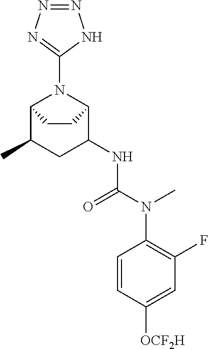 |
| | | 1-(4-(difluoromethoxy)-2- |
| | | fluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 57″* | 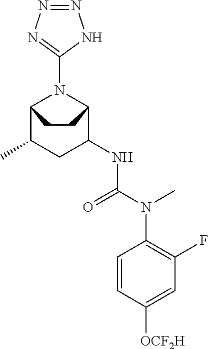 |
| | | 1-(4-(difluoromethoxy)-2- |
| | | fluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 57A* | 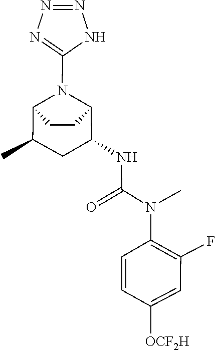 |
| | | 1-(4-(difluoromethoxy)-2- |
| | | fluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 57B* | 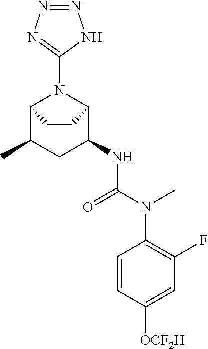 |
| | | 1-(4-(difluoromethoxy)-2- |
| | | fluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 57C* | 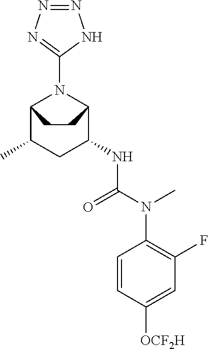 |
| | | 1-(4-(difluoromethoxy)-2- |
| | | fluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 57D* | 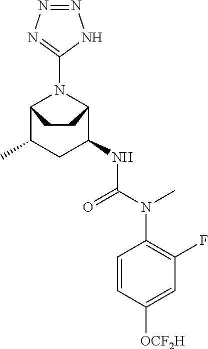 |
| | | 1-(4-(difluoromethoxy)-2- |
| | | fluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-58 | 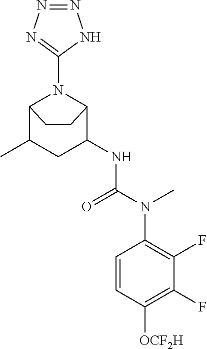 |
| | | 1-(4-(difluoromethoxy)-2,3- |
| | | difluorophenyl)-1-methyl-3-(4- |
| | | methyl-8-(1H-tetrazol-5-yl)-8- |
| | | azabicyclo[3.2.1]octan-2-yl)urea |
| TA | 58′* | 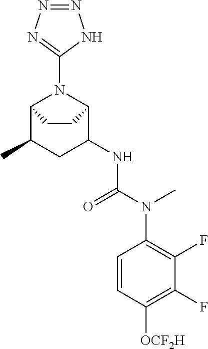 |
| | | 1-(4-(difluoromethoxy)-2,3- |
| | | difluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 58″* | 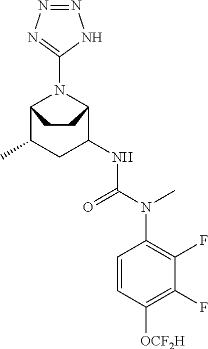 |
| | | 1-(4-(difluoromethoxy)-2,3- |
| | | difluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 58A* | 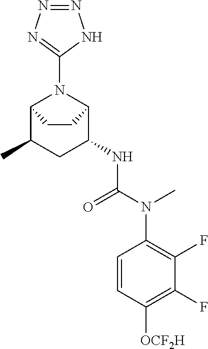 |
| | | 1-(4-(difluoromethoxy)-2,3- |
| | | difluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 58B* | 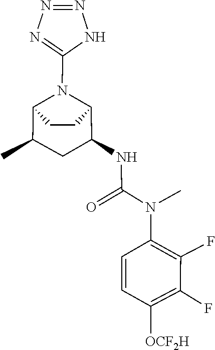 |
| | | 1-(4-(difluoromethoxy)-2,3- |
| | | difluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 58C* | 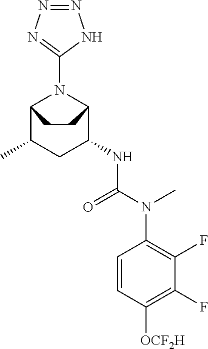 |
| | | 1-(4-(difluoromethoxy)-2,3- |
| | | difluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 58D* | 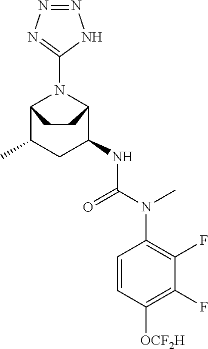 |
| | | 1-(4-(difluoromethoxy)-2,3- |
| | | difluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-59 | 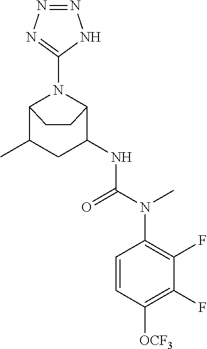 [SMILES: CC1CC(NC(=O)N(C)c2ccc(OC(F)(F)F)c(F)c2F)C2CCC1N2c1nnn[nH]1] |
| | | 1-(2,3-difluoro-4- |
| | | (trifluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 59′* | 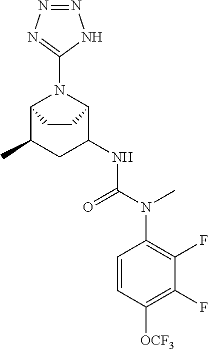 [SMILES: C[C@@H]1CC(NC(=O)N(C)c2ccc(OC(F)(F)F)c(F)c2F)[C@H]2CC[C@@H]1N2c1nnn[nH]1] |
| | | 1-(2,3-difluoro-4- |
| | | (trifluoromethoxy)phenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 59″* | 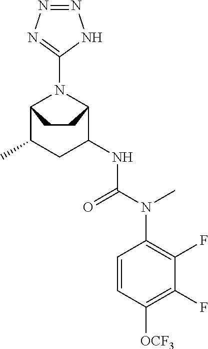 [SMILES: C[C@H]1CC(NC(=O)N(C)c2ccc(OC(F)(F)F)c(F)c2F)[C@@H]2CC[C@H]1N2c1nnn[nH]1] |
| | | 1-(2,3-difluoro-4- |
| | | (trifluoromethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 59A* | 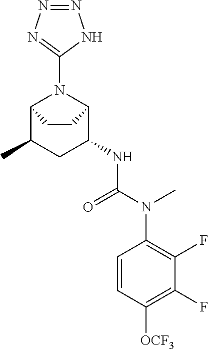 [SMILES: C[C@@H]1C[C@@H](NC(=O)N(C)c2ccc(OC(F)(F)F)c(F)c2F)[C@H]2CC[C@@H]1N2c1nnn[nH]1] |
| | | 1-(2,3-difluoro-4- |
| | | (trifluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 59B* | 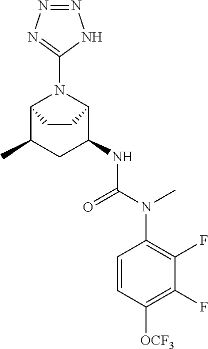 [SMILES: C[C@@H]1C[C@H](NC(=O)N(C)c2ccc(OC(F)(F)F)c(F)c2F)[C@H]2CC[C@@H]1N2c1nnn[nH]1] |
| | | 1-(2,3-difluoro-4- |
| | | (trifluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 59C* | 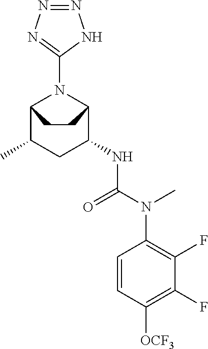 [SMILES: C[C@H]1C[C@@H](NC(=O)N(C)c2ccc(OC(F)(F)F)c(F)c2F)[C@@H]2CC[C@H]1N2c1nnn[nH]1] |
| | | 1-(2,3-difluoro-4- |
| | | (trifluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 59D* | 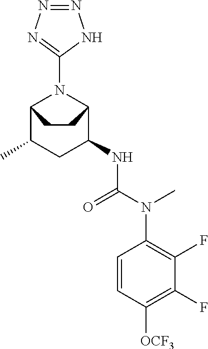 [SMILES: C[C@H]1C[C@H](NC(=O)N(C)c2ccc(OC(F)(F)F)c(F)c2F)[C@@H]2CC[C@H]1N2c1nnn[nH]1] |
| | | 1-(2,3-difluoro-4- |
| | | (trifluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | Rac-60 | 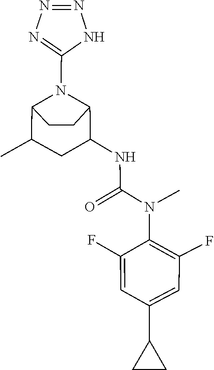 [SMILES: CC1CC(NC(=O)N(C)c2c(F)cc(C3CC3)cc2F)C2CCC1N2c1nnn[nH]1] |
| | | 1-(4-cyclopropyl-2,6- |
| | | difluorophenyl)-1-methyl-3-(4- |
| | | methyl-8-(1H-tetrazol-5-yl)-8- |
| | | azabicyclo[3.2.1]octan-2-yl)urea |
| TA | 60′* | 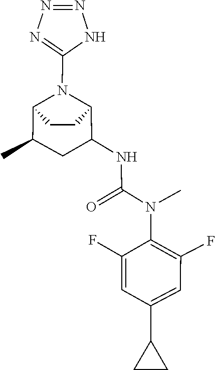 [SMILES: C[C@@H]1CC(NC(=O)N(C)c2c(F)cc(C3CC3)cc2F)[C@H]2CC[C@@H]1N2c1nnn[nH]1] |
| | | 1-(4-cyclopropyl-2,6- |
| | | difluorophenyl)-1-methyl-3- |
| | | ((1R,2R,5S)-4-methyl-8-(1H- |
| | | tetrazol-5-yl)-8- |
| | | azabicyclo[3.2.1]octan-2-yl)urea |
| TA | 60″* | 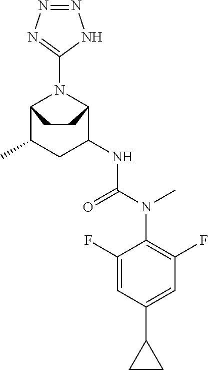 [SMILES: C[C@H]1CC(NC(=O)N(C)c2c(F)cc(C3CC3)cc2F)[C@@H]2CC[C@H]1N2c1nnn[nH]1] |
| | | 1-(4-cyclopropyl-2,6- |
| | | difluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 60A* | 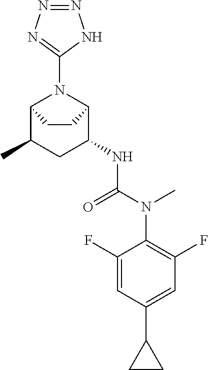 [SMILES: C[C@@H]1C[C@@H](NC(=O)N(C)c2c(F)cc(C3CC3)cc2F)[C@H]2CC[C@@H]1N2c1nnn[nH]1] |
| | | 1-(4-cyclopropyl-2,6- |
| | | difluorophenyl)-1-methyl-3- |
| | | ((1R,2R,4R,5S)-4-methyl-8-(1H- |
| | | tetrazol-5-yl)-8- |
| | | azabicyclo[3.2.1]octan-2-yl)urea |
| TA | 60B* | 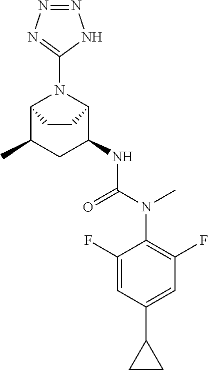 [SMILES: C[C@@H]1C[C@H](NC(=O)N(C)c2c(F)cc(C3CC3)cc2F)[C@H]2CC[C@@H]1N2c1nnn[nH]1] |
| | | 1-(4-cyclopropyl-2,6- |
| | | difluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 60C* | 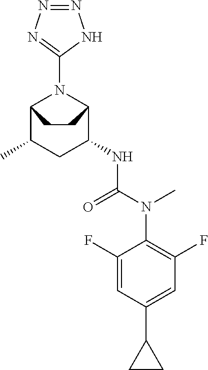 [SMILES: C[C@H]1C[C@@H](NC(=O)N(C)c2c(F)cc(C3CC3)cc2F)[C@@H]2CC[C@H]1N2c1nnn[nH]1] |
| | | 1-(4-cyclopropyl-2,6- |
| | | difluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 60D* | 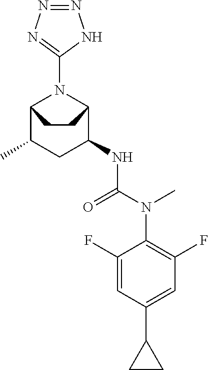 [SMILES: C[C@H]1C[C@H](NC(=O)N(C)c2c(F)cc(C3CC3)cc2F)[C@@H]2CC[C@H]1N2c1nnn[nH]1] |
| | | 1-(4-cyclopropyl-2,6- |
| | | difluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-61 | 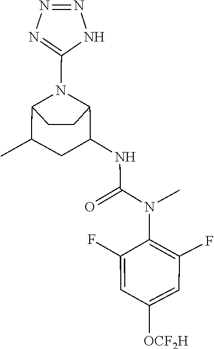 |
| | | 1-(4-(difluoromethoxy)-2,6- |
| | | difluorophenyl)-1-methyl-3-(4- |
| | | methyl-8-(1H-tetrazol-5-yl)-8- |
| | | azabicyclo[3.2.1]octan-2-yl)urea |
| TA | 61′* | 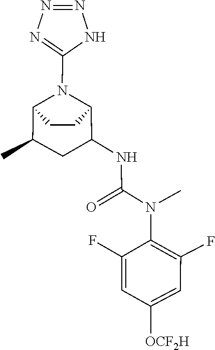 |
| | | 1-(4-(difluoromethoxy)-2,6- |
| | | difluorophenyl)-1-methyl-3- |
| | | (1R,2R,5S)-4-methyl-8-(1H- |
| | | tetrazol-5-yl)-8- |
| | | azabicyclo[3.2.1]octan-2-yl)urea |
| TA | 61″* | 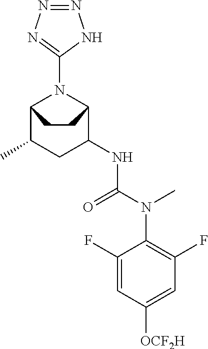 |
| | | 1-(4-(difluoromethoxy)-2,6- |
| | | difluorophenyl)-1-methyl-3- |
| | | ((1S,2S,5R)-4-methyl-8-(1H- |
| | | tetrazol-5-yl)-8- |
| | | azabicyclo[3.2.1]octan-2-yl)urea |
| TA | 61A* | 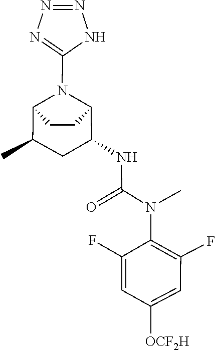 |
| | | 1-(4-(difluoromethoxy)-2,6- |
| | | difluorophenyl)-1-methyl-3- |
| | | ((1R,2R,4R,S)-4-methyl-8-(1H- |
| | | tetrazol-5-yl)-8- |
| | | azabicyclo[3.2.1]octan-2-yl)urea |
| TA | 61B* | 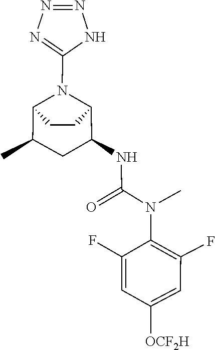 |
| | | 1-(4-(difluoromethoxy)-2,6- |
| | | difluorophenyl)-1-methyl-3- |
| | | ((1R,2S,4R,5S)-4-methyl-8-(1H- |
| | | tetrazol-5-yl)-8- |
| | | azabicyclo[3.2.1]octan-2-yl)urea |
| TA | 61C* | 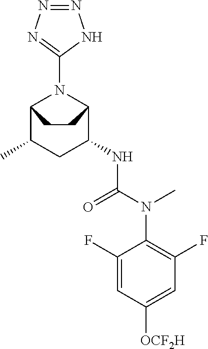 |
| | | 1-(4-(difluromethoxy)-2,6- |
| | | difluorophenyl)-1-methyl-3- |
| | | ((1S,2R,4S,5R)-4-methyl-8-(1H- |
| | | tetrazol-5-yl)-8- |
| | | azabicyclo[3.2.1]octan-2-yl)urea |
| TA | 61D* | 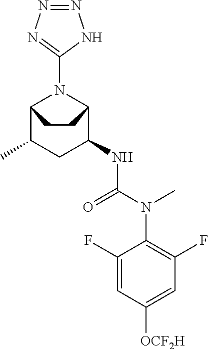 |
| | | 1-(4-(difluoromethoxy)-2,6- |
| | | difluorophenyl)-1-methyl-3- |
| | | ((1S,2S,4S,5R)-4-methyl-8-(1H- |
| | | tetrazol-5-yl)-8- |
| | | azabicyclo[3.2.1]octan-2-yl)urea |
| TA | Rac-62 | 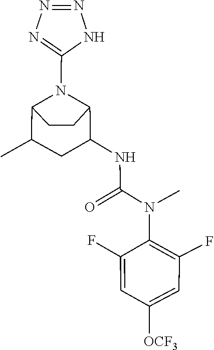 [SMILES: CC1CC(NC(=O)N(C)c2c(F)cc(OC(F)(F)F)cc2F)C2CCC1N2c1nnn[nH]1] |
| | | 1-(2,6-difluoro-4- |
| | | (trifluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 62′* | 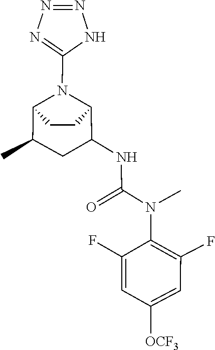 [SMILES: C[C@@H]1CC(NC(=O)N(C)c2c(F)cc(OC(F)(F)F)cc2F)[C@H]2CC[C@@H]1N2c1nnn[nH]1] |
| | | 1-(2,6-difluoro-4- |
| | | (trifluoromethoxy)phenyl)-1- |
| | | methyl-3-((1R,2R,5S)-4-methyl-8- |
| | | 1H-tetrazol-5-yl)-8- |
| | | azabicyclo[3.2.1]octan-2-yl)urea |
| TA | 62″* | 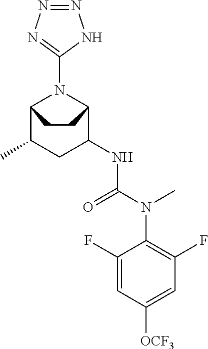 [SMILES: C[C@H]1CC(NC(=O)N(C)c2c(F)cc(OC(F)(F)F)cc2F)[C@@H]2CC[C@H]1N2c1nnn[nH]1] |
| | | 1-(2,6-difluoro-4- |
| | | (trifluoromethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 62A* | 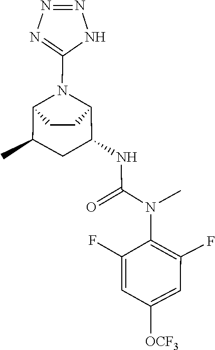 [SMILES: C[C@@H]1C[C@@H](NC(=O)N(C)c2c(F)cc(OC(F)(F)F)cc2F)[C@H]2CC[C@@H]1N2c1nnn[nH]1] |
| | | 1-(2,6-difluoro-4- |
| | | (trifluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 62B* | 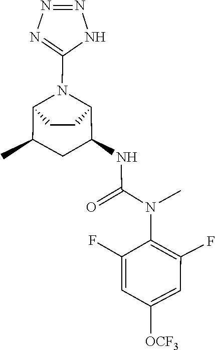 [SMILES: C[C@@H]1C[C@H](NC(=O)N(C)c2c(F)cc(OC(F)(F)F)cc2F)[C@H]2CC[C@@H]1N2c1nnn[nH]1] |
| | | 1-(2,6-difluoro-4- |
| | | (trifluoromethoxy)phenyl)-1- |
| | | methyl-3-((1R,2S,4R,5S)-4-methyl-1- |
| | | 8-(1H-tetrazol-5-yl)-8- |
| | | azabicyclo[3.2.1]octan-2-yl)urea |
| TA | 62C* | 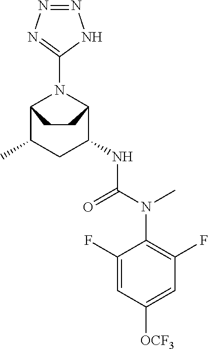 [SMILES: C[C@H]1C[C@@H](NC(=O)N(C)c2c(F)cc(OC(F)(F)F)cc2F)[C@@H]2CC[C@H]1N2c1nnn[nH]1] |
| | | 1-(2,6-difluoro-4- |
| | | (trifluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 62D* | 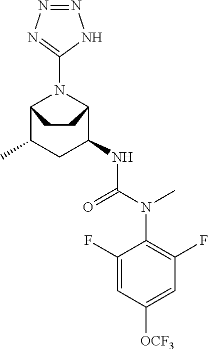 [SMILES: C[C@H]1C[C@H](NC(=O)N(C)c2c(F)cc(OC(F)(F)F)cc2F)[C@@H]2CC[C@H]1N2c1nnn[nH]1] |
| | | 1-(2,6-difluoro-4- |
| | | (trifluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-y)urea |
| TA | Rac-63 | 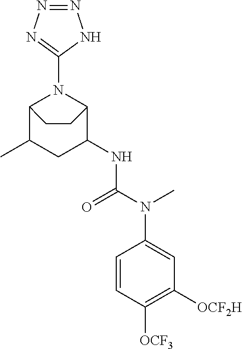 |
| | | 1-(3-(difluoromethoxy)-4- |
| | | (trifluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 63′* | 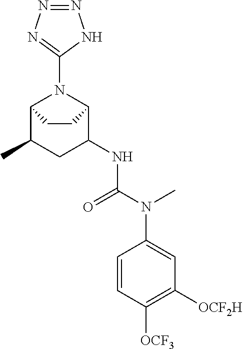 |
| | | 1-(3-(difluoromethoxy)-4- |
| | | (trifluoromethoxy)phenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 63″* | 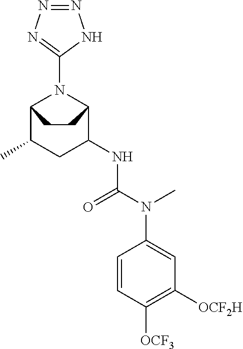 |
| | | 1-(3-(difluoromethoxy)-4- |
| | | (trifluoromethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 63A* | 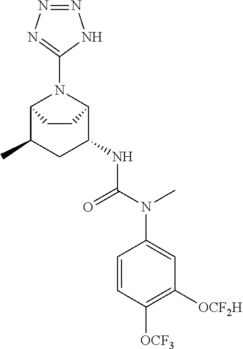 |
| | | 1-(3-(difluormethoxy)-4- |
| | | (trifluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 63B* | 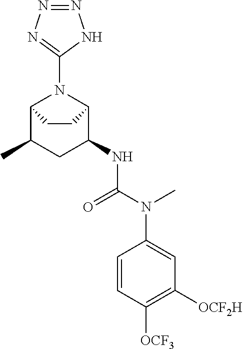 |
| | | 1-(3-(difluoromethoxy)-4- |
| | | (trifluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 63C* | 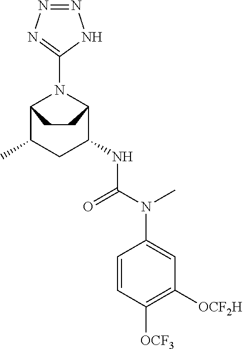 |
| | | 1-(3-(difluoromethoxy)-4- |
| | | (trifluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 63D* | 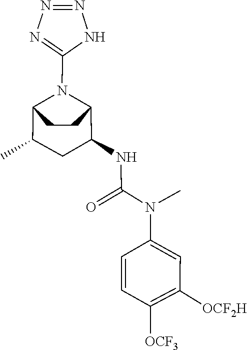 |
| | | 1-(3-(difluoromethoxy)-4- |
| | | (trifluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-y)urea |
| TA | Rac-64 | 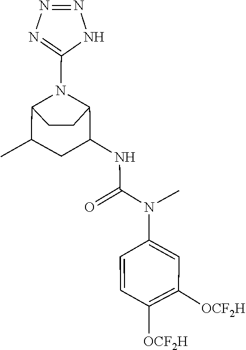 |
| | | 1-(3,4- |
| | | bis(difluoromethoxy)phenyl)-1- |
| | | methyl-3-(4-methyl-8-(1H-tetrazol- |
| | | 5-yl)-8-azabicyclo[3.2.1]octan-2- |
| | | yl)urea |
| TA | 64′* | 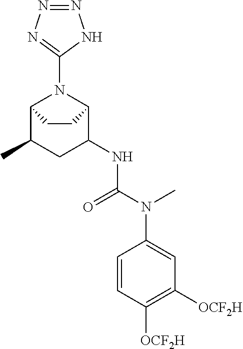 |
| | | 1-(3,4- |
| | | bis(difluoromethoxy)phenyl)-1- |
| | | methyl-3-((1R,2R,5S)-4-methyl-8- |
| | | (1H-tetrazol-5-yl)-8- |
| | | azabicyclo[3.2.1]octan-2-yl)urea |
| TA | 64″* | 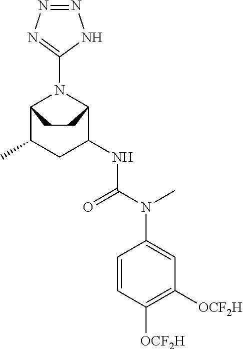 |
| | | 1-(3,4- |
| | | bis(difluroomethoxy)phenyl)-1- |
| | | methyl-3-((1S,2S,5R)-4-methyl-8- |
| | | (1H-tetrazol-5-yl)-8- |
| | | azabicyclo[3.2.1]octan-2-yl)urea |
| TA | 64A* | 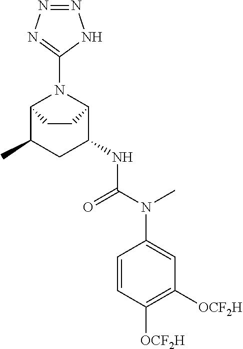 |
| | | 1-(3,4- |
| | | bis(difluoromethoxy)phenyl)-1- |
| | | methyl-3-((1R,2R,4R,5S)-4-methyl- |
| | | 8-(1H-tetrazol-5-yl)-8- |
| | | azabicyclo[3.2.1]octan-2-yl)urea |
| TA | 64B* | 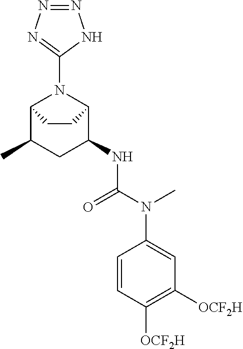 |
| | | 1-(3,4- |
| | | bis(difluoromethoxy)phenyl)-1- |
| | | methyl-3-((1R,2S,4R,5S)-4-methyl- |
| | | 8-(1H-tetrazol-5-yl)-8- |
| | | azabicyclo[3.2.1]octan-2-yl)urea |
| TA | 64C* | 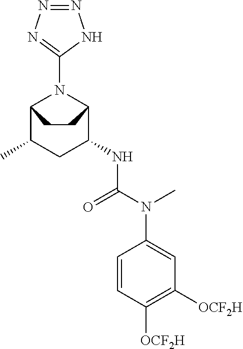 |
| | | 1-(3,4- |
| | | bis(difluoromethoxy)phenyl)-1- |
| | | methyl-3-((1S,2R,4S,5R)-4-methyl- |
| | | 8-(1H-tetrazol-5-yl)-8- |
| | | azabicyclo[3.2.1]octan-2-yl)urea |
| TA | 64D* | 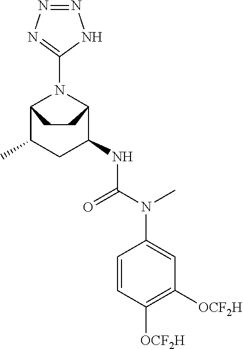 |
| | | 1-(3,4- |
| | | bis(difluoromethoxy)phenyl)-1- |
| | | methyl-3-((1S,2S,4S,5R)-4-methyl- |
| | | 8-(1H-tetrazol-5-yl)-8- |
| | | azabicyclo[3.2.1]octan-2-yl)urea |
| TA | 41D* | 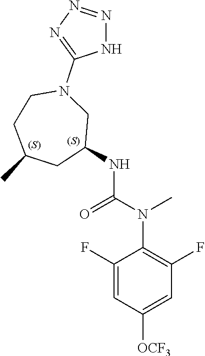 |
| | | 1-(2,6-difluoro-4- |
| | | (trifluoromethoxy)phenyl)-1- |
| | | methyl-3-((3S,5S)-5-methyl-1-(1H- |
| | | tetrazol-5-yl)azepan-3-yl)urea |
| TA | 65′* | 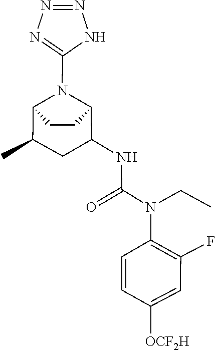 |
| | | 1-(4-(difluoromethoxy)-2- |
| | | fluorophenyl)-1-ethyl-3-((1R, |
| | | 4R,5S)-4-methyl-8-(1H- |
| | | tetrazol-5-yl)-8- |
| | | azabicyclo[3.2.1]octan-2- |
| | | yl)urea |
| TA | 65″* | 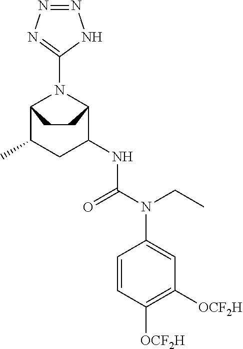 |
| | | 1-(4-(difluoromethoxy)-2- |
| | | fluorophenyl)-1-ethyl-3-((1S, |
| | | 4S,5R)-4-methyl-8-(1H- |
| | | tetrazol-5-yl)-8- |
| | | azabicyclo[3.2.1]octan-2- |
| | | yl)urea |
| TA | 65B* | 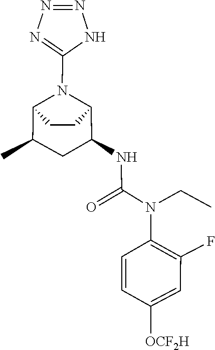 |
| | | 1-(4-(difluoromethoxy)-2- |
| | | fluorophenyl)-1-ethyl-3- |
| | | ((1R,2S,4R,5S)-4-methyl-8- |
| | | (1H-tetrazol-5-yl)-8- |
| | | azabicyclo[3.2.1]octan-2- |
| | | yl)urea |
| TA | 65C* | 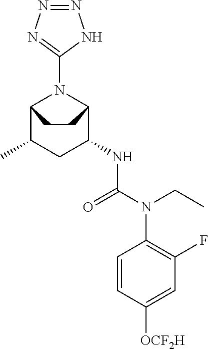 |
| | | 1-(4-(difluoromethoxy)-2- |
| | | fluorophenyl)-1-ethyl-3- |
| | | ((1S,2R,4S,5R)-4-methyl-8- |
| | | (1H-tetrazol-5-yl)-8- |
| | | azabicyclo[3.2.1]octan-2- |
| | | yl)urea |

In certain embodiments, the compound is selected from the group consisting of Compound 6A and Compound 39A, or a pharmaceutically acceptable salt of tautomer of any of the foregoing.
In certain embodiments, the compound is selected from the group consisting of Compound 4A*, Compound 5A*, Compound 7A*, Compound 8A*, Compound 10A*, Compound 11A*, Compound 13A*, Compound 15A*, Compound 16A*, Compound 17A*, Compound 18A*, Compound 19A*, Compound 20A*, Compound 21A*, Compound 22A*, Compound 23A*, Compound 24A*, Compound 25A*, Compound 40A*, Compound 41A*, and Compound 43A*, or a pharmaceutically acceptable salt or tautomer of any of the foregoing.
In certain embodiments, the compound is selected from the group consisting of Compound 42A*, Compound 43A*, Compound 45A*, Compound 46A*, Compound 47A*, Compound 48A*, Compound 49A*, Compound 51A*, Compound 52A*, Compound 54A*, Compound 55A*, Compound 56A*, Compound 57A*, Compound 58A*, Compound 60A*, Compound 61A*, Compound 62A*, Compound 64A*, and Compound 65A*, or a pharmaceutically acceptable salt or tautomer of any of the foregoing.

### (ii) Pharmaceutical Compositions

Pharmaceutical compositions comprising a compound of Formula (I-B), or a pharmaceutically acceptable salt or tautomer thereof, and a pharmaceutically acceptable carrier, are further contemplated herein.

For example, in some aspects, provided is a pharmaceutical composition comprising a compound of Formula (I-B), or a pharmaceutically acceptable salt or tautomer thereof, and a pharmaceutically acceptable carrier.

Exemplary pharmaceutical acceptable carriers may include diluents, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine.

Administration to the subject can be accomplished via any mode of administration, for example, by oral administration, topical administration, or by injection. Depending on the intended mode of administration, the pharmaceutical composition comprising the compound of Formula (I-B), or a pharmaceutically acceptable salt or tautomer thereof, can be in solid, semi-solid or liquid dosage form.

A compound of Formula (I-B), or a pharmaceutically acceptable salt or tautomer thereof, may be administered alone in the pharmaceutical composition as the sole therapeutic agent, or may be administered in combination with another therapeutic agent. Combination treatment may be achieved by way of co-administration (e.g., the two agents being administered at the same time) or sequential administration (e.g., one agent being administered first, then the other). In the case of co-administration, the compound of Formula (I-B), or a pharmaceutically acceptable salt or tautomer thereof, may be administered in the same pharmaceutical composition as the other therapeutic agent, or may be administered in a separate pharmaceutical composition. The choice of the other therapeutic agent will depend upon the diagnosis of the attending physicians and their judgment of the condition of the subject and the appropriate treatment protocol.

### (iii) Methods of Treatment

Compounds of Formula (I-B), and pharmaceutically acceptable salts and tautomers thereof, have been found useful as inhibitors of NLRP3 activity.

In some aspects, provided is a method of treating a disease or disorder in a subject in need thereof, comprising administering to the subject a compound of Formula (I-B), or a pharmaceutically acceptable salt or tautomer thereof, or a pharmaceutical composition comprising same. In some aspects, provided is a method of treating a disease or disorder in a subject in need thereof, comprising administering to the subject an effective amount of a compound of Formula (I-B), or a pharmaceutically acceptable salt or tautomer thereof, or a pharmaceutical composition comprising same. In some embodiments, the disease or disorder is associated with aberrant NLRP3 activity, and the method comprises inhibiting the aberrant NLRP3 activity such that the subject is treated.

In some embodiments, the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, obesity, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

In some embodiments, the disease or disorder is a disease or disorder of central nervous system and/or peripheral nervous system (“PNS”), such as dementia, Alzheimer's disease (“AD”) epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), Huntington's disease (“HD”), spinal cord injury, or obesity related to a neuroinflammation.

In some embodiments, the disease or disorder is a primary neurological disease of the muscle, such as a dystrophy or spinal muscular atrophy.

In some embodiments, the disease or disorder is an inflammatory disorder, such as gout or anemia of inflammation.

In some embodiments, the disease or disorder is an autoimmune disease, such as ulcerative colitis.

In some embodiments, the disease or disorder is cancer, such as skin cancer or colon cancer.

In some embodiments, the disease or disorder is an infection, such as a neuro-infection.

In some embodiments, the disease or disorder is a metabolic disease, such as diabetes, e.g., type 2 diabetes.

In some embodiments, the disease or disorder is obesity. In some embodiments, the obesity is related to neuroinflammation, e.g., hypothalamic inflammation and/or gliosis. In some embodiments, the obesity is related to a metabolic disorder.

In some embodiments, the disease or disorder is a cardiovascular disease, such as stroke, atherosclerosis or atherosclerotic cardiovascular disease (ASCVD).

In some embodiments, the disease or disorder is a respiratory disease, such as asthma (e.g., steroid-resistant asthma, severe steroid-resistant asthma) or chronic obstructive pulmonary disease (“COPD”).

In some embodiments, the disease or disorder is a kidney disease, such as acute kidney disease, a chronic kidney disease, or a rare kidney disease. In certain embodiments, chronic kidney disease is chronic kidney failure.

In some embodiments, the disease or disorder is a liver disease, such as nonalcoholic fatty liver disease (NAFLD) or non-alcoholic steatohepatitis (NASH, also known as MASH or metabolic dysfunction-associated steatohepatitis).

In some embodiments, the disease or disorder is an ocular disease, such as optic neuritis or macular degeneration.

In some embodiments, the disease or disorder is a skin disease, such as psoriasis, hidradenitis suppurativa (HS), or atopic dermatitis.

In some embodiments, the disease or disorder is a lymphatic disease.

In some embodiments, the disease or disorder is a rheumatic disease, such as osteoarthritis, dermatomyositis, Still's disease, or juvenile idiopathic arthritis.

In some embodiments, the disease or disorder is a psychological disease, such as a neuropsychiatric condition, including depression, major depressive disorder, or refractory depression.

In some embodiments, the disease or disorder is a graft versus host disease.

In some embodiments, the disease or disorder is pain (including disorders related to pain management), such as pain management addiction, osteoarthritis pain, or allodynia.

In some embodiments, the NLRP3-related disease in a subject that has been determined to carry a germline or somatic non-silent mutation in NLRP3 is cryopyrin-associated autoinflammatory syndrome. In some embodiments, the cryopyrin-associated autoinflammatory syndrome is familial cold autoinflammatory syndrome, Muckle-Wells syndrome, or neonatal onset multisystem inflammatory disease (NOMID).

In some embodiments, the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), spinal cord injury, dystrophies, neuro-infections, pain management addiction, neuropsychiatric conditions (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, hidradenitis suppurativa, or obesity related neuroinflammation.

In other aspects, provided is a method of modulating (e.g., inhibiting) NLRP3 activity (e.g., in vitro or in vivo in a cell, or in a subject), comprising contacting the cell with or administering to the subject a compound of Formula (I-B), or a pharmaceutically acceptable salt or tautomer thereof. In certain the compound or a pharmaceutically acceptable salt or tautomer thereof is administered to the cell or subject in an effective amount.

### (iv) Methods of Preparation

Compounds of Formula (I-B), and salts and tautomers thereof, may be synthesized following General Schemes A or B, as provided below. The Examples further described non-limiting examples of this general syntheses.

### General Method, Protocol A

A suitable general route for the preparation of compounds described herein follows Protocol A as depicted in General Scheme A.

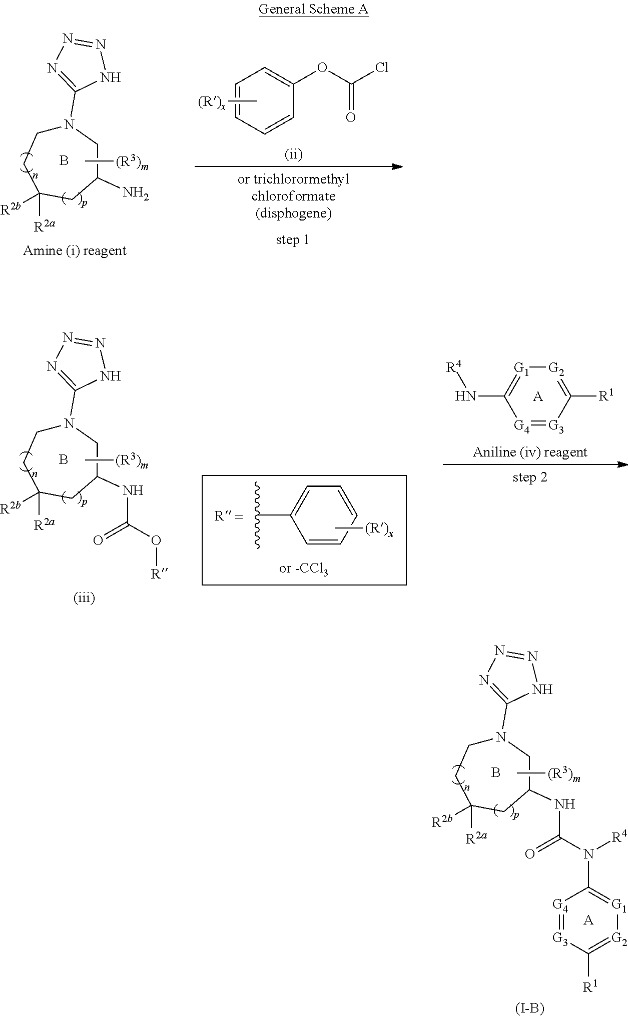

Step one involves reaction of the amine (i) reagent, or a salt or tautomer thereof, with a phenyl carbonochloridate (ii), wherein R′ is —NO₂ or halogen, and x is 0, 1, or 2, or disphosgene, to provide carbamate (iii), or a salt or tautomer thereof. Step 2 involves coupling of the carbamate (iii), or a salt thereof, with an aniline (iv) reagent, or salt thereof, to provide a compound of Formula (I-B), or a salt or tautomer thereof.

### General Method, Protocol B

Another suitable general route for the preparation of compounds described herein follows Protocol B as depicted in General Scheme B.

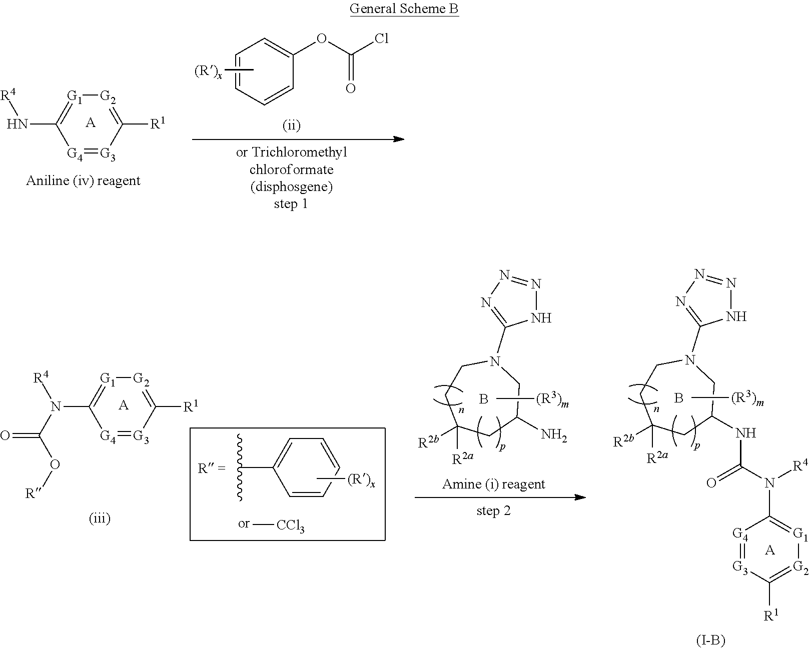

Step one involves reaction of the aniline (iv) reagent, or a salt thereof, with a phenyl carbonochloridate (ii), or salt thereof, wherein R′ is —NO₂ or halogen, and x is 0, 1, or 2, or disphosgene, to provide carbamate (iii), or salt thereof. Step 2 involves coupling of the carbamate (iii), or a salt thereof, with an amine (i) reagent, or a salt or tautomer thereof, to provide a compound of Formula (I-B), or a salt or tautomer thereof.

### (v) Biological Assays

Various in vitro or in vivo biological assays may be suitable for detecting the effect of the compounds of the present disclosure. These in vitro or in vivo biological assays can include, but are not limited to, binding assays, cellular assays (cell lines, primary cells and whole blood), in vitro cell viability assays, as well as assays for determining NLRP3 binding activity and potency, unbound clearance, solubility, and permeability.

In some embodiments, the compounds of the instant disclosure may be tested for their human-NLRP3 inhibitory activity/potency using known procedures, such as the methodology reported in Coll et al. Nat Med. (2015) 21(3):248-255. See also the Examples, Biological Assay Methods section.

In some embodiments, the compounds may be tested for unbound clearance (Clu) following known procedures, such as described in Miller et al., J. Med. Chem. (2020) 63:12156-12170. For example, unbound clearance (Clu) may be calculated by dividing total clearance (‘CL’ in mL/min/kg) as measured in blood or plasma by the unbound fraction in plasma (fu).

In some embodiments, the solubility of the compounds may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two hour incubation period, followed by filtration, and reported in mM by LC-MS/MS. Thermodynamic solubility in physiologically relevant media may be measured by LC-MS/MS, after a twenty-four hour incubation, followed by filtration, and reported in mg/mL.

### (vi) Exemplary Embodiments

Additional Exemplary Embodiments are as set forth below. Other embodiments are contemplated in the claims.

Exemplary Embodiment 1. A compound of Formula (I-B):

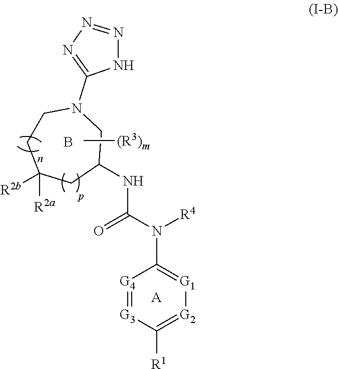

or a pharmaceutically acceptable salt or tautomer thereof;
wherein:
Ring A is a ring system wherein:
   G₁ is CR^{G1} or N; G₂ is CR^{G2} or N; G₃ is CR^{G3} or N; and G₄ is CR^{G4} or N; provided no more than two of G₁, G₂, G₃, and G₄ are N;
   R¹ is halo, C₁₋₆ alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆ haloalkyl, —OR^{G5}, —SR^{G5}, or —N(R^{G5})₂,
   or R¹ and G₂, together with the atoms to which they are attached, are joined to form a 5-membered heteroaryl ring independently substituted with 0, 1, 2, or 3 R^{G7};
   R^{G1}, R^{G2}, R^{G3}, and R^{G4} are each independently selected from the group consisting of hydrogen, halo, C₁₋₆ alkyl, C₁₋₆ haloalkyl, and —OR^{G6}; and
   R^{G5} and R^{G6} are each independently hydrogen, C₁₋₆ alkyl, or C₁₋₆ haloalkyl;
   each instance of R^{G7} is independently halo, C₁₋₆ alkyl, C₁₋₆ haloalkyl, —OR^{G5}, —SR^{G5}, and —N(R^{G5})₂; and
Ring B is a ring system wherein:
   n is 0 or 1;
   p is 1 or 2;
   m is 0, 1, 2, or 3;
   each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo;
   each instance of R³ is independently halo, C₁₋₆ alkyl or C₁₋₆ haloalkyl, or two R³ groups may be joined to form a C₁₋₃ alkylene bridging group or C₁₋₃ haloalkylene bridging group between the two atoms to which they are attached; and
   R⁴ is hydrogen, C₁₋₃ alkyl, C₃-C₄ carbocyclyl, or C₃-C₄ carbocyclyl-C₁₋₃ alkyl-, wherein the alkyl and carbocyclyl are each independently substituted with 0, 1, 2, 3, 4, 5, or 6 halo, and wherein the carbocyclyl is further independently substituted with 0, 1, or 2 C₁₋₃ alkyl or C₁₋₃ haloalkyl.

Exemplary Embodiment 2. The compound of Exemplary Embodiment 1, wherein the compound is of Formula:

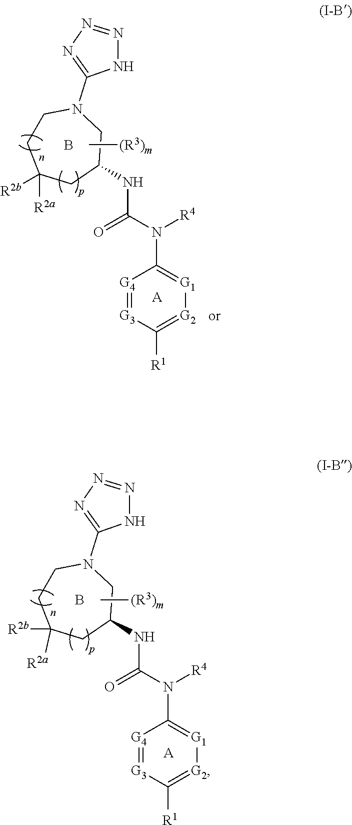

or a pharmaceutically acceptable salt or tautomer thereof.

Exemplary Embodiment 3. The compound of Exemplary Embodiment 2, wherein the compound is of Formula:

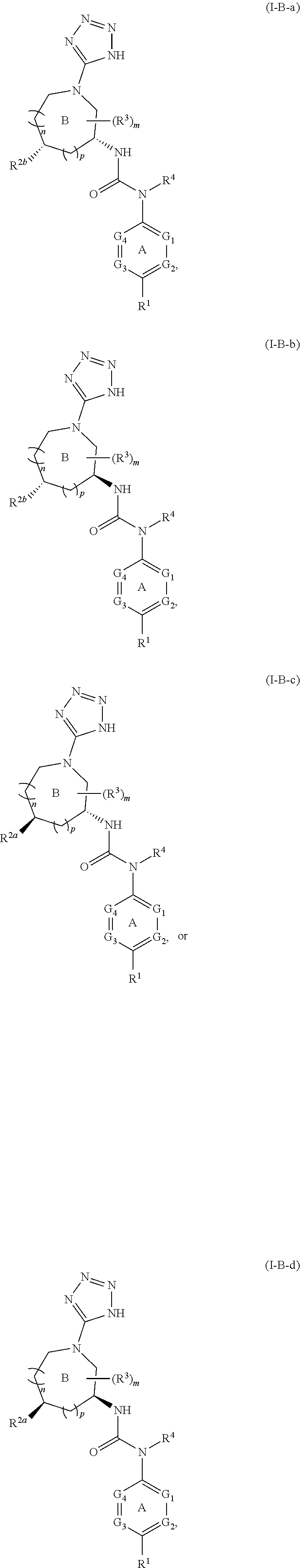

or a pharmaceutically acceptable salt or tautomer thereof.

Exemplary Embodiment 4. The compound of Exemplary Embodiment 1, wherein the compound is of the Formula:

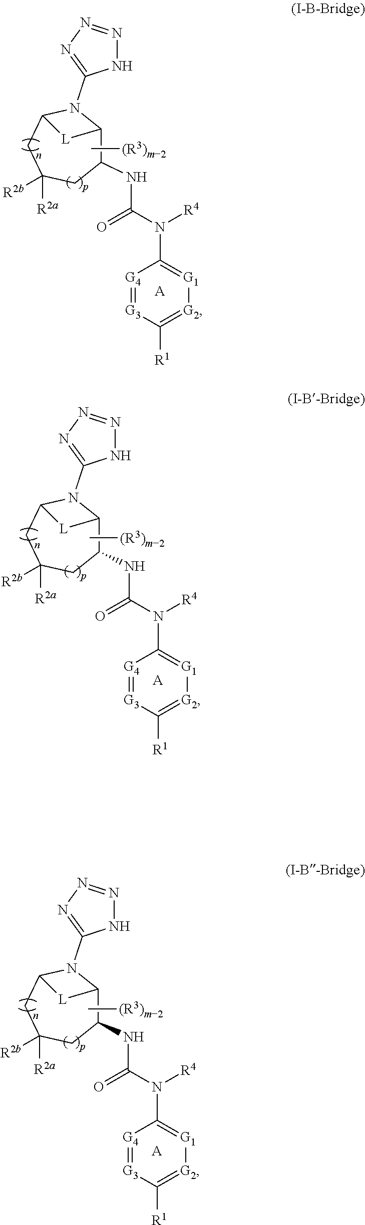

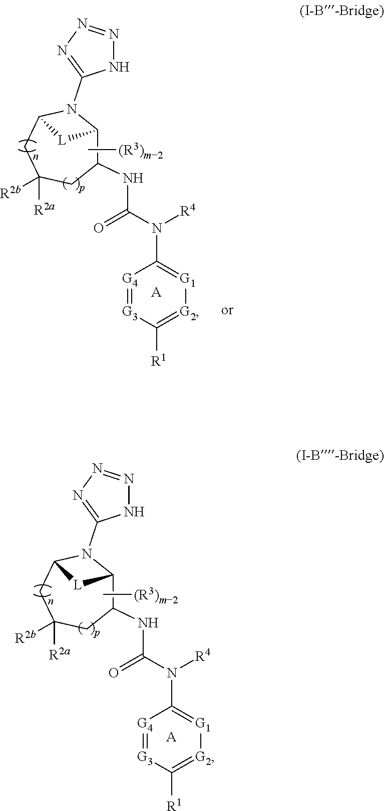

or a pharmaceutically acceptable salt or tautomer thereof, wherein L is a C₁₋₃ alkylene bridging group or C₁₋₃ haloalkylene bridging group.

Exemplary Embodiment 5. The compound of Exemplary Embodiment 4, wherein the compound is of the Formula:

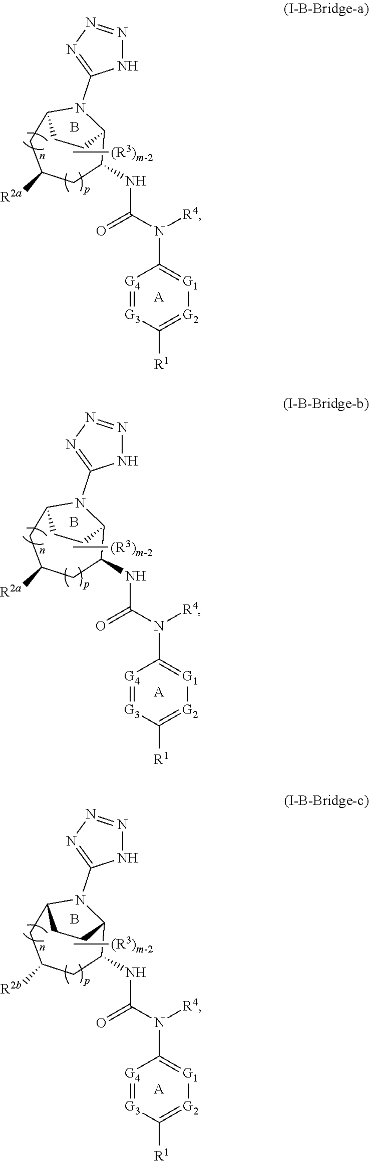

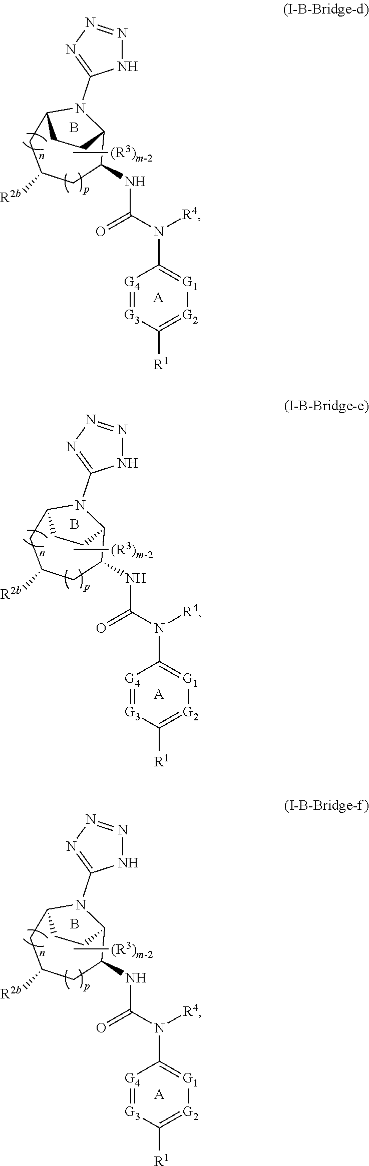

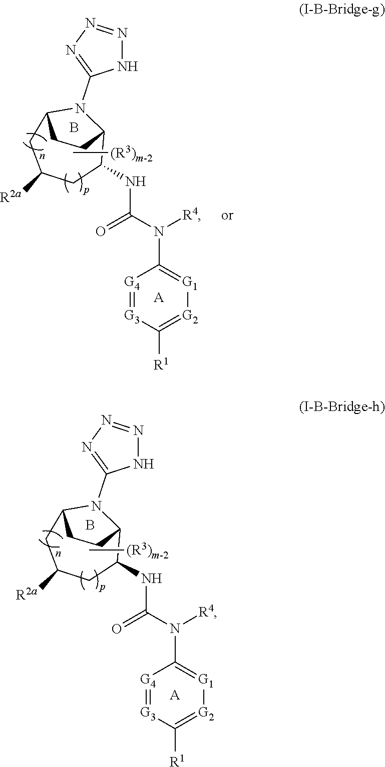

or a pharmaceutically acceptable salt or tautomer thereof.

Exemplary Embodiment 6. The compound of any one of Exemplary Embodiments 1-5, or a pharmaceutically acceptable salt or tautomer thereof, wherein R⁴ is not hydrogen.

Exemplary Embodiment 7. The compound of any one of Exemplary Embodiments 1-6, or a pharmaceutically acceptable salt or tautomer thereof, wherein Ring A is a ring system wherein:
R¹ is halo, C₁₋₆ haloalkyl, —OR^{G5}, or C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂
or R¹ and G₂, together with the atoms to which they are attached, are joined to form a 5-membered heteroaryl ring independently substituted with 0, 1, 2, or 3 R^{G7};
R^{G1}, R^{G2}, R^{G3}, and R^{G4} are each independently selected from the group consisting of hydrogen, halo, and —OR^{G6};
R^{G5} and R^{G6} are each independently hydrogen or C₁₋₆ haloalkyl; and
each instance of R^{G7} is independently halo, C₁₋₆ alkyl, C₁₋₆ haloalkyl, —OR^{G5}, —SR^{G5}, and —N(R^{G5})₂.

Exemplary Embodiment 8. The compound of Exemplary Embodiment 7, or a pharmaceutically acceptable salt or tautomer thereof, wherein Ring A is a ring system wherein:
R¹ is Cl, cyclopropyl, CF₃, CF₂H, OCF₃, or OCF₂H,
or R¹ and G₂, together with the atoms to which they are attached, are joined to form oxazole, isoxazole, pyrazole, or imidazole; and
R^{G1}, R^{G2}, R^{G3}, and R^{G4} are each independently selected from the group consisting of H, F, Cl, OH, and OCF₂H.

Exemplary Embodiment 9. The compound of any one of Exemplary Embodiments 1-6, or a pharmaceutically acceptable salt or tautomer thereof, wherein Ring B is a ring system wherein:
each instance of R^{2a} and R^{2b} is independently hydrogen or C₁₋₆ alkyl;
two R³ groups may be joined to form a C₁₋₃ alkylene bridging group between the two atoms to which they are attached; and
R⁴ is C₁₋₃ alkyl, C₃-C₄ carbocyclyl, or C₃-C₄ carbocyclyl-C₁₋₃ alkyl, wherein the alkyl and carbocyclyl are each independently substituted with 0, 1, 2, 3, 4, 5, or 6 halo.

Exemplary Embodiment 10. The compound of Exemplary Embodiment 9, or a pharmaceutically acceptable salt or tautomer thereof, wherein Ring B is a ring system wherein:
each instance of R^{2a} and R^{2b} is independently hydrogen or methyl;
two R³ groups may be joined to form an ethylene bridging group between the two atoms to which they are attached; and
R⁴ is methyl, ethyl, cyclopropyl, or cyclopropyl-methyl.

Exemplary Embodiment 11. The compound of any one of Exemplary Embodiments 1-10, or a pharmaceutically acceptable salt or tautomer thereof, wherein G₁ is CR^{G1}, G₂ is CR^{G2}, G₃ is CR^{G3}, and G₄ is CR^{G4}.

Exemplary Embodiment 12. The compound of Exemplary Embodiment 11, or a pharmaceutically acceptable salt or tautomer thereof, wherein G₁ is CH, G₂ is CH, G₃ is CH, and G₄ is CH.

Exemplary Embodiment 13. The compound of Exemplary Embodiment 11, or a pharmaceutically acceptable salt or tautomer thereof, wherein G₁ is CR^{G1}, G₂ is CH, G₃ is CH, and G₄ is CH.

Exemplary Embodiment 14. The compound of Exemplary Embodiment 11, or a pharmaceutically acceptable salt or tautomer thereof, wherein G₁ is CH, G₂ is CR^{G2}, G₃ is CH, and G₄ is CH.

Exemplary Embodiment 15. The compound of Exemplary Embodiment 11, or a pharmaceutically acceptable salt or tautomer thereof, wherein G₁ is CR^{G1}, G₂ is CR^{G2}, G₃ is CH, and G₄ is CH.

Exemplary Embodiment 16. The compound of Exemplary Embodiment 11, or a pharmaceutically acceptable salt or tautomer thereof, wherein G₁ is CR^{G1}, G₂ is CH, G₃ is CH, and G₄ is CR^{G4}.

Exemplary Embodiment 17. The compound of any one of Exemplary Embodiments 1-10, or a pharmaceutically acceptable salt or tautomer thereof, wherein G₁ is N, G₂ is CR^{G2}, G₃ is CR^{G3}, and G₄ is CR^{G4}.

Exemplary Embodiment 18. The compound of Exemplary Embodiment 17, or a pharmaceutically acceptable salt or tautomer thereof, wherein G₁ is N, G₂ is CH, G₃ is CH, and G₄ is CH.

Exemplary Embodiment 19. The compound of any one of Exemplary Embodiments 1-10, or a pharmaceutically acceptable salt or tautomer thereof, wherein G₁ is CR^{G1}, G₂ is N, G₃ is CR^{G3}, and G₄ is CR^{G4}.

Exemplary Embodiment 20. The compound of Exemplary Embodiment 19, or a pharmaceutically acceptable salt or tautomer thereof, wherein G₁ is CH, G₂ is N, G₃ is CH, and G₄ is CH.

Exemplary Embodiment 21. The compound of Exemplary Embodiment 19, or a pharmaceutically acceptable salt or tautomer thereof, wherein G₁ is CR^{G1}, G₂ is N, G₃ is CH, and G₄ is CH.

Exemplary Embodiment 22. The compound of any one of Exemplary Embodiments 1-10, or a pharmaceutically acceptable salt or tautomer thereof, wherein G₁ is CR^{G1}, G₂ is CR^{G2}, G₃ is N, and G₄ is CR^{G4}.

Exemplary Embodiment 23. The compound of Exemplary Embodiment 22, or a pharmaceutically acceptable salt or tautomer thereof, wherein G₁ is CR^{G1}, G₂ is CH, G₃ is N, and G₄ is CH.

Exemplary Embodiment 24. The compound of any one of Exemplary Embodiments 1-10, or a pharmaceutically acceptable salt or tautomer thereof, wherein G₁ is CR^{G1}, G₂ is CR^{G2}, G₃ is CR^{G3}, and G₄ is N.

Exemplary Embodiment 25. The compound of Exemplary Embodiment 24, or a pharmaceutically acceptable salt or tautomer thereof, wherein G₁ is CR^{G1}, G₂ is CH, G₃ is CH, and G₄ is N.

Exemplary Embodiment 26. The compound of any one of Exemplary Embodiments 1-10, or a pharmaceutically acceptable salt or tautomer thereof, wherein G₁ is N, G₂ is CR^{G2}, G₃ is CR^{G3}, and G₄ is N.

Exemplary Embodiment 27. The compound of Exemplary Embodiment 26, or a pharmaceutically acceptable salt or tautomer thereof, wherein G₁ is N, G₂ is CH, G₃ is CH, and G₄ is N.

Exemplary Embodiment 28. The compound of any one of Exemplary Embodiments 1-10, or a pharmaceutically acceptable salt or tautomer thereof, wherein G₁ is N, G₂ is CR^{G2}, G₃ is N, and G₄ is CR^{G4}.

Exemplary Embodiment 29. The compound of Exemplary Embodiment 28, or a pharmaceutically acceptable salt or tautomer thereof, wherein G₁ is N, G₂ is CH, G₃ is N, and G₄ is CH.

Exemplary Embodiment 30. The compound of any one of Exemplary Embodiments 1-10, or a pharmaceutically acceptable salt or tautomer thereof, wherein G₁ is CR^{G1}, G₂ is N, G₃ is N, and G₄ is CR^{G4}.

Exemplary Embodiment 31. The compound of Exemplary Embodiment 30, or a pharmaceutically acceptable salt or tautomer thereof, wherein G₁ is CH, G₂ is N, G₃ is N, and G₄ is CH.

Exemplary Embodiment 32. The compound of any one of Exemplary Embodiments 1-10, or a pharmaceutically acceptable salt or tautomer thereof, wherein G₁ is CR^{G1}, G₂ is CR^{G2}, G₃ is N, and G₄ is N.

Exemplary Embodiment 33. The compound of Exemplary Embodiment 32, or a pharmaceutically acceptable salt or tautomer thereof, wherein G₁ is CH, G₂ is CH, G₃ is N, and G₄ is N.

Exemplary Embodiment 34. The compound of any one of Exemplary Embodiments 1-10, or a pharmaceutically acceptable salt or tautomer thereof, wherein G₁ is CR^{G1}, G₄ is CR^{G4}, and R¹ and G₂, together with the atoms to which they are attached, are joined to form a 5-membered heteroaryl ring independently substituted with 0, 1, 2, or 3 R^{G7}.

Exemplary Embodiment 35. The compound of Exemplary Embodiment 34, or a pharmaceutically acceptable salt or tautomer thereof, wherein G₁ is CH and G₄ is CH.

Exemplary Embodiment 36. The compound of any one of Exemplary Embodiments 1-10, or a pharmaceutically acceptable salt or tautomer thereof, wherein R¹ is halo, C₁₋₆ haloalkyl, —OR^{G5}, or C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂.

Exemplary Embodiment 37. The compound of Exemplary Embodiment 36, or a pharmaceutically acceptable salt t or tautomer hereof, wherein R¹ is —Cl, cyclopropyl, —CF₃, —CF₂H, —OCF₃, or —OCF₂H.

Exemplary Embodiment 38. The compound of any one of Exemplary Embodiments 1-10, or a pharmaceutically acceptable salt or tautomer thereof, wherein R¹ and G₂, together with the atoms to which they are attached, are joined to form a 5-membered heteroaryl ring independently substituted with 0, 1, 2, or 3 R^{G7}.

Exemplary Embodiment 39. The compound of Exemplary Embodiment 38, or a pharmaceutically acceptable salt or tautomer thereof, wherein R¹ and G₂, together with the atoms to which they are attached, are joined to form an oxazole, isoxazole, pyrazole, or imidazole.

Exemplary Embodiment 40. The compound of any one of Exemplary Embodiments 1-10, or a pharmaceutically acceptable salt or tautomer thereof, wherein R^{G1}, R^{G2}, R^{G3}, and R^{G4} are each independently selected from the group consisting of hydrogen, halo, and —OR^{G6}.

Exemplary Embodiment 41. The compound of Exemplary Embodiment 40, or a pharmaceutically acceptable salt or tautomer thereof, wherein R^{G1}, R^{G2}, R^{G3}, and R^{G4} are each independently selected from the group consisting of hydrogen, F, Cl, OH, and OCF₂H.

Exemplary Embodiment 42. The compound of any one of Exemplary Embodiments 1-10, or a pharmaceutically acceptable salt or tautomer thereof, wherein R^{G5} and R^{G6} are each independently hydrogen or C₁₋₆ haloalkyl.

Exemplary Embodiment 43. The compound of Exemplary Embodiment 42, or a pharmaceutically acceptable salt or tautomer thereof, wherein R^{G5} is CF₃ or CF₂H.

Exemplary Embodiment 44. The compound of Exemplary Embodiment 42, or a pharmaceutically acceptable salt or tautomer thereof, wherein R^{G6} is hydrogen or CF₂H.

Exemplary Embodiment 45. The compound of any one of Exemplary Embodiments 1-44, or a pharmaceutically acceptable salt or tautomer thereof, wherein each instance of R^{2a} and R^{2b} is independently hydrogen or C₁₋₆ alkyl.

Exemplary Embodiment 46. The compound of Exemplary Embodiment 45, or a pharmaceutically acceptable salt or tautomer thereof, wherein each instance of R^{2a} and R^{2b} is independently hydrogen or methyl.

Exemplary Embodiment 47. The compound of any one of Exemplary Embodiments 1-46, or a pharmaceutically acceptable salt or tautomer thereof, wherein R⁴ is hydrogen, C₁₋₃ alkyl, C₃-C₄ carbocyclyl, or C₃-C₄ carbocyclyl-C₁₋₃ alkyl, wherein the alkyl and carbocyclyl are each independently substituted with 0, 1, 2, 3, 4, 5, or 6 halo as valency permits.

Exemplary Embodiment 48. The compound of Exemplary Embodiment 47, or a pharmaceutically acceptable salt or tautomer thereof, wherein R⁴ is methyl, ethyl, cyclopropyl, or cyclopropyl-methyl.

Exemplary Embodiment 49. The compound of any one of Exemplary Embodiments 1-48, or a pharmaceutically acceptable salt or tautomer thereof, wherein two R³ groups may be joined to form a C₁₋₃ alkylene bridging group between the two atoms to which they are attached.

Exemplary Embodiment 50. The compound of Exemplary Embodiment 49, or a pharmaceutically acceptable salt or tautomer thereof, wherein two R³ groups may be joined to form an ethylene bridging group between the two atoms to which they are attached.

Exemplary Embodiment 51. The compound of any one of Exemplary Embodiments 1-50, or a pharmaceutically acceptable salt or tautomer thereof, wherein n is 1.

Exemplary Embodiment 52. The compound of any one of Exemplary Embodiments 1-50, or a pharmaceutically acceptable salt or tautomer thereof, wherein n is 0.

Exemplary Embodiment 53. The compound of any one of Exemplary Embodiments 1-50, or a pharmaceutically acceptable salt or tautomer thereof, wherein p is 1.

Exemplary Embodiment 54. The compound of any one of Exemplary Embodiments 1-50, or a pharmaceutically acceptable salt or tautomer thereof, wherein m is 0.

Exemplary Embodiment 55. The compound of any one of Exemplary Embodiments 1-50, or a pharmaceutically acceptable salt or tautomer thereof, wherein m is 1.

Exemplary Embodiment 56. The compound of any one of Exemplary Embodiments 1-50, or a pharmaceutically acceptable salt or tautomer thereof, wherein m is 2.

Exemplary Embodiment 57. The compound of any one of Exemplary Embodiments 1-50, or a pharmaceutically acceptable salt or tautomer thereof, wherein Ring A of formula:

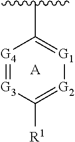

is a group of formula:

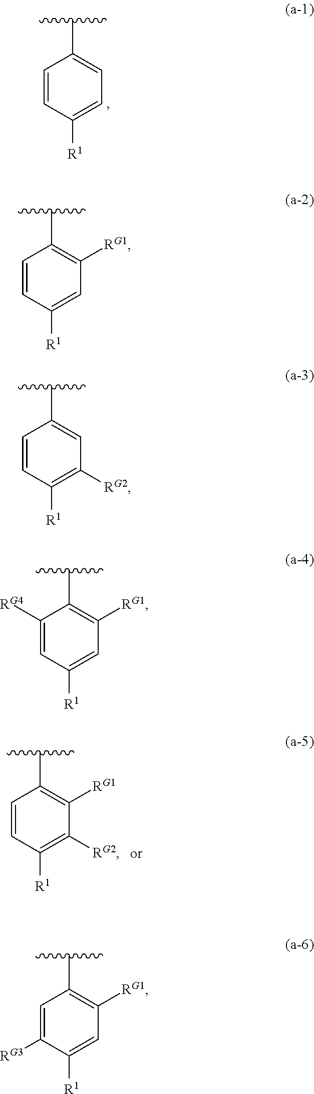

wherein R^{G1}, R^{G2}, R^{G3}, and R^{G4} are each independently selected from the group consisting of halo, C₁₋₆ alkyl, C₁₋₆ haloalkyl, and —OR^{G6}.

Exemplary Embodiment 58. The compound of Exemplary Embodiment 57, or a pharmaceutically acceptable salt or tautomer thereof, wherein Ring A of formula (a-2), (a-4), (a-5), (a-6) is a group of formula:

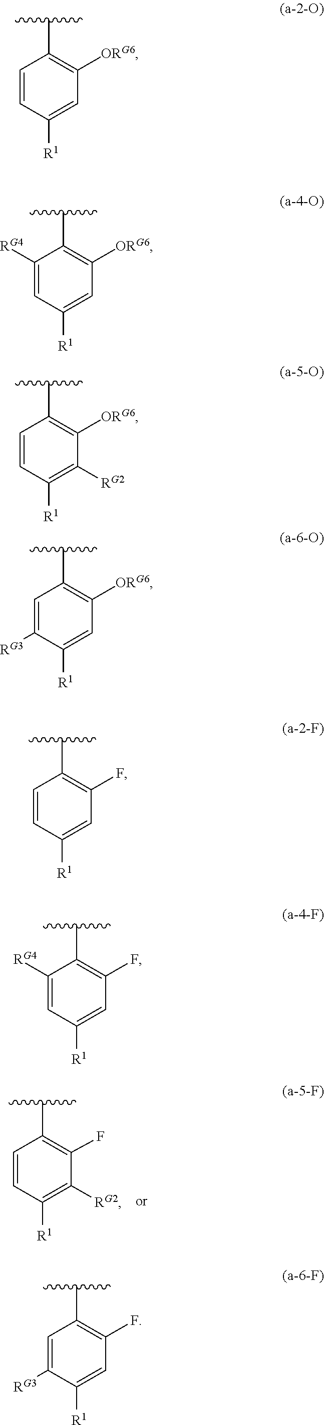

Exemplary Embodiment 59. The compound of any one of Exemplary Embodiments 1-50, or a pharmaceutically acceptable salt or tautomer thereof, wherein Ring A of formula:

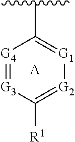

is a group of formula:

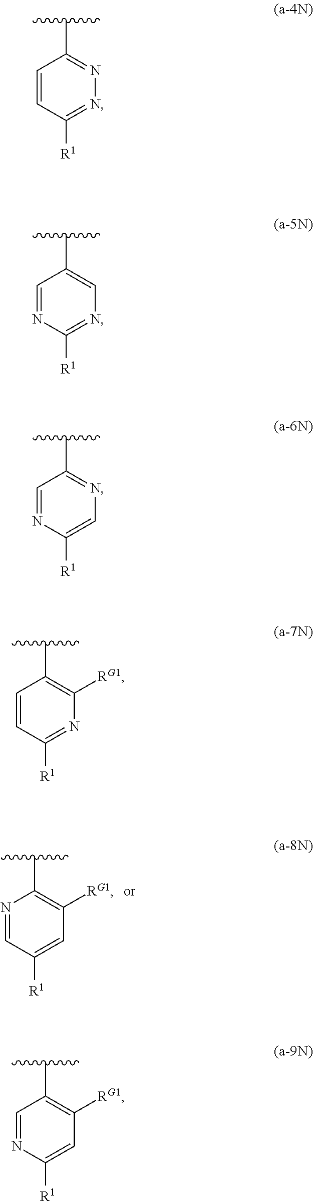

wherein R^{G1} is halo, C₁₋₆alkyl, C₁₋₆haloalkyl, or —OR^{G6}.

Exemplary Embodiment 60. The compound of Exemplary Embodiment 59, or a pharmaceutically acceptable salt or tautomer thereof, wherein Ring A of formula (a-7N), (a-8N), or (a-9N) is of the formula:

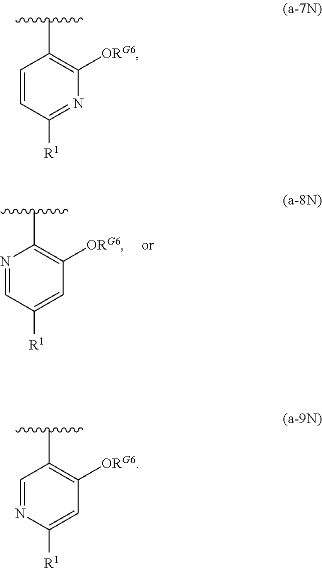

Exemplary Embodiment 61. The compound of any one of Exemplary Embodiments 1-50, or a pharmaceutically acceptable salt or tautomer thereof, wherein Ring A of formula:

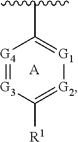

and R¹ and G₂, together with the atoms to which they are attached, are joined to form a 5-membered heteroaryl ring, wherein the Ring A, R¹, and G² provide a group of formula:

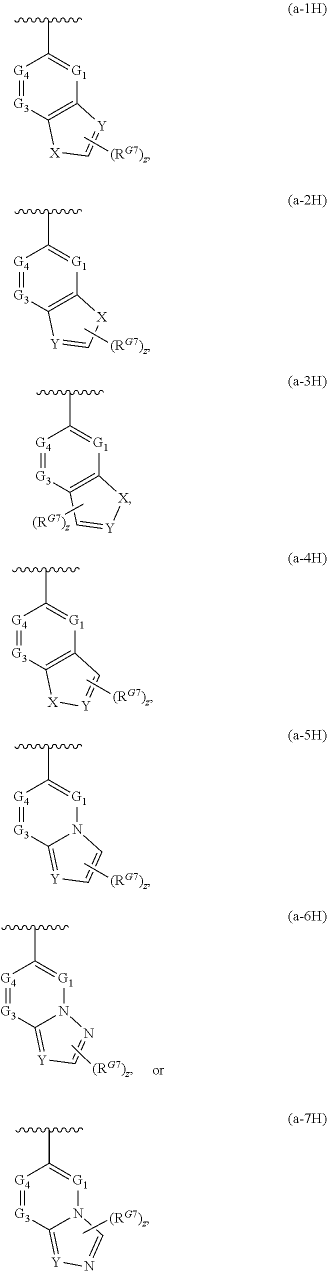

wherein:
   X is O, S, NH, or NR^{G7}.
   Y is N, CH, or CR^{G7}; and
   z is 0 or 1;
   provided if R^{G7} is a group attached to a nitrogen (N) atom, then R^{G7} is C₁₋₆ alkyl or C₁₋₆ haloalkyl.

Exemplary Embodiment 62. The compound of Exemplary Embodiment 61, or a pharmaceutically acceptable salt or tautomer thereof, wherein Ring A, R¹, and G₂, together with the atoms to which they are attached, are joined to form a 5-membered heteroaryl ring, wherein the group is of formula:

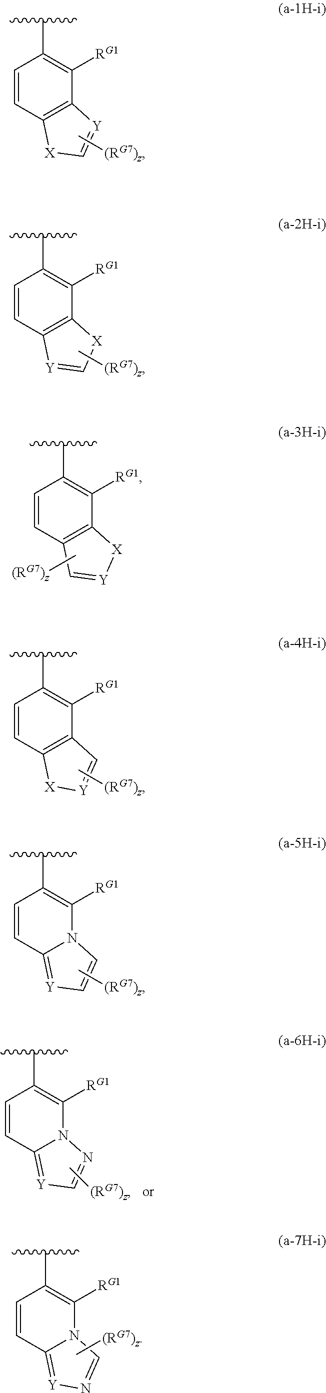

Exemplary Embodiment 63. The compound of any one of Exemplary Embodiments 1-50, or a pharmaceutically acceptable salt or tautomer thereof, wherein Ring A is a group of formula:

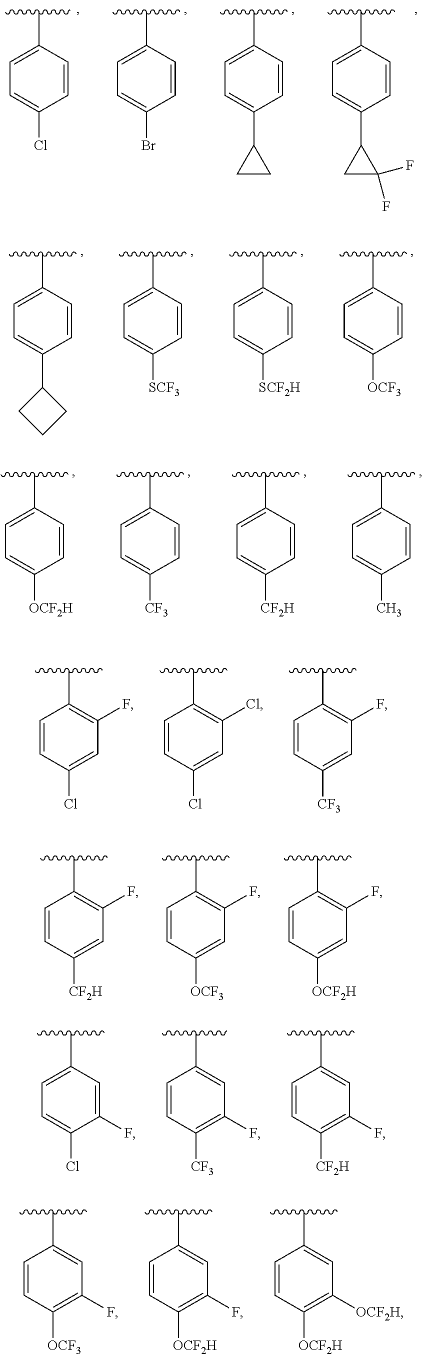

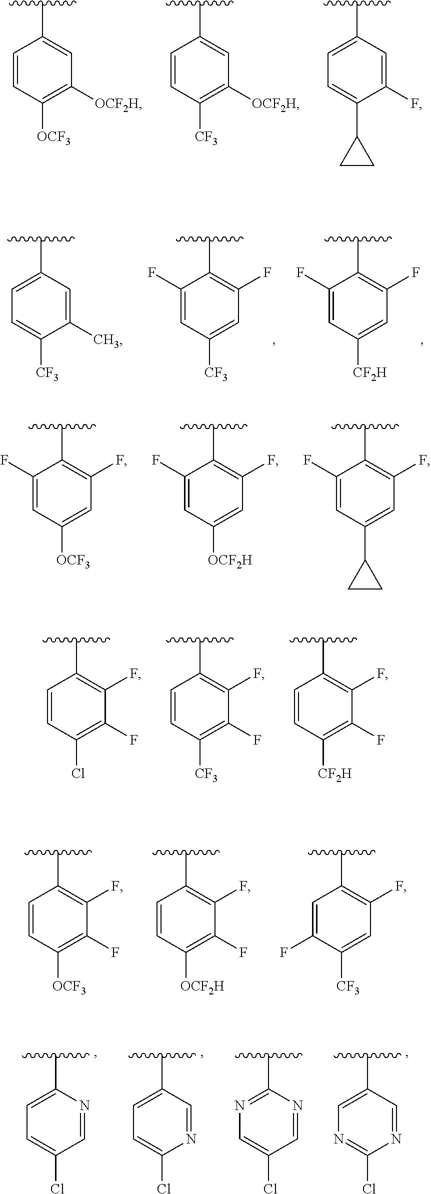

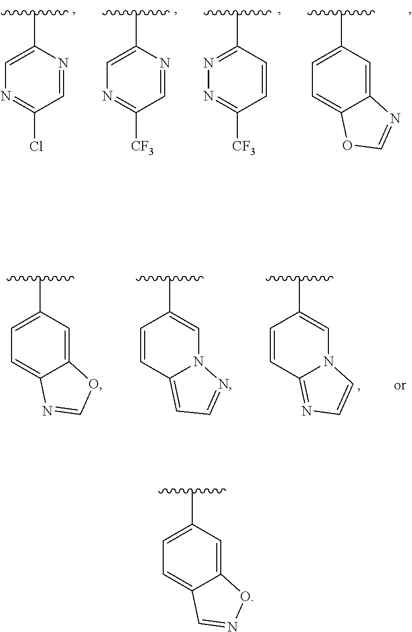

Exemplary Embodiment 64. The compound of any one of Exemplary Embodiments 1-50, or a pharmaceutically acceptable salt or tautomer thereof, wherein Ring A is a group of formula:

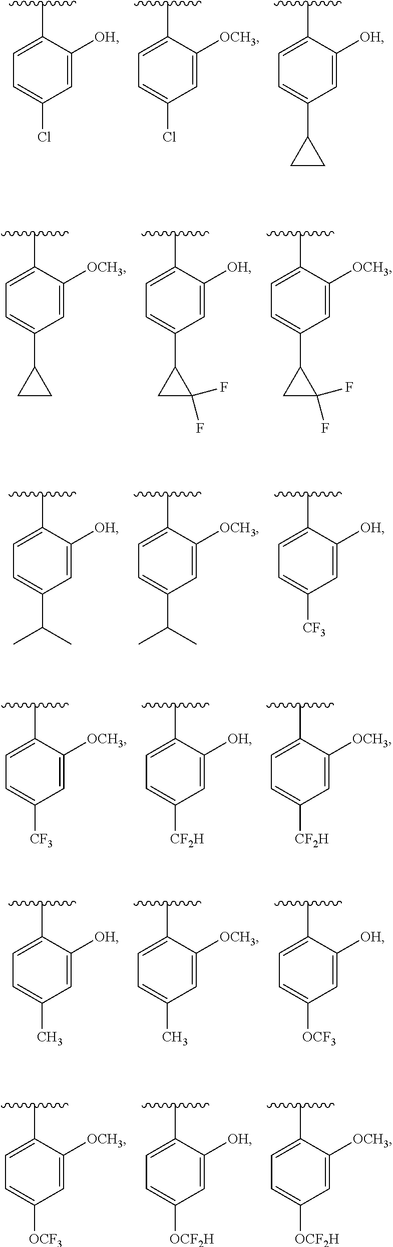

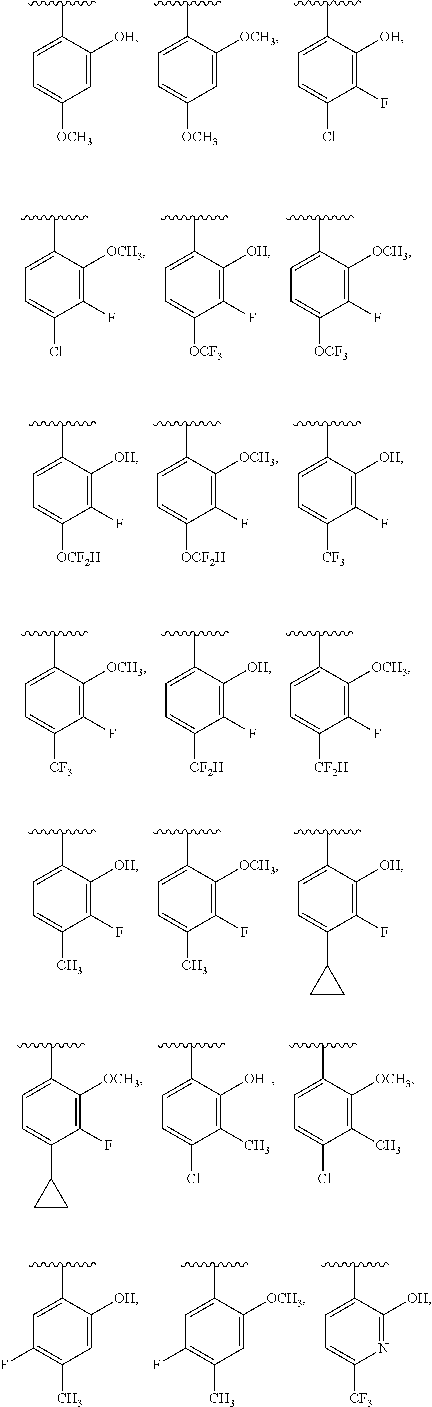

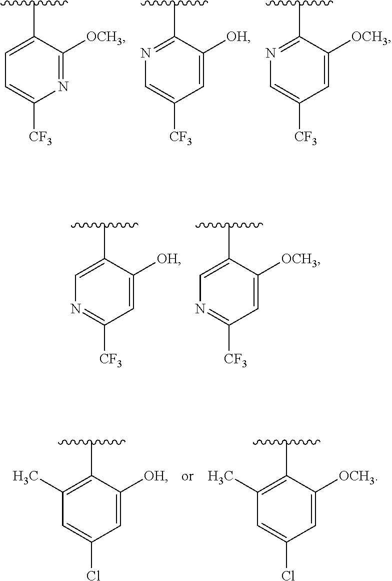

Exemplary Embodiment 65. The compound of any one of Exemplary Embodiments 1-50, or a pharmaceutically acceptable salt or tautomer thereof, wherein Ring B of formula:

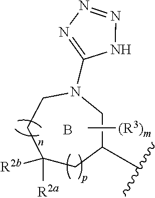

is a group of formula:

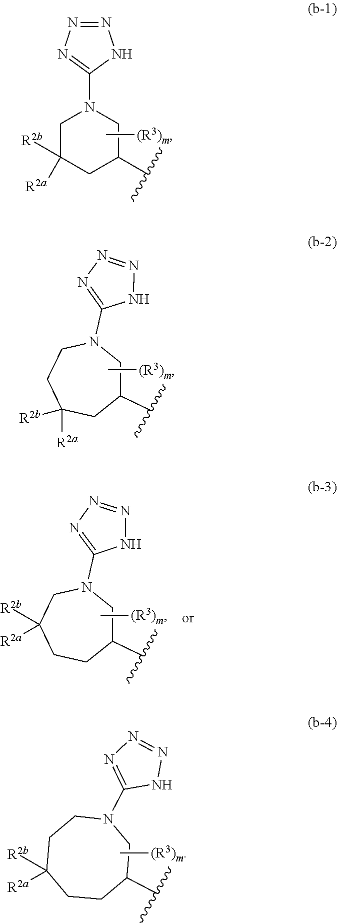

Exemplary Embodiment 66. The compound of Exemplary Embodiment 65, or a pharmaceutically acceptable salt or tautomer thereof, wherein Ring B is a group of formula:

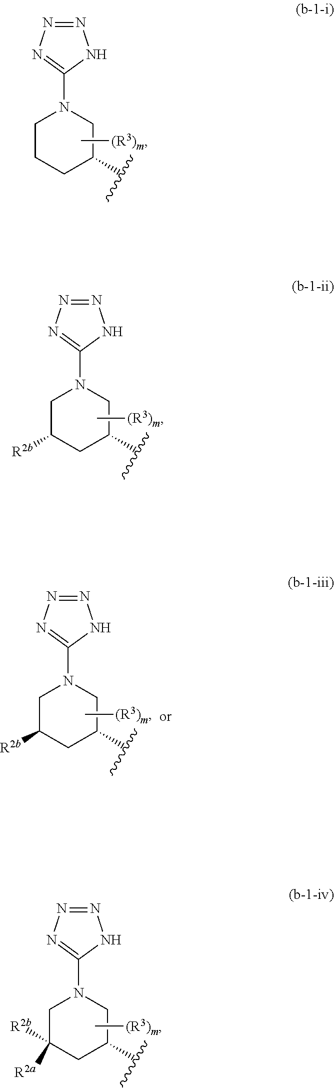

wherein each instance of R^{2a} and R^{2b} is independently halo, C₁₋₆ alkyl, C₁₋₆haloalkyl, C₃-C₄ carbocyclyl, or 3-4 membered heterocyclyl.

Exemplary Embodiment 67. The compound of Exemplary Embodiment 65, or a pharmaceutically acceptable salt or tautomer thereof, wherein Ring B of formula (b-1), (b-2), (b-3), or (b-4), when two R³ groups are joined to form a C₁₋₃ alkylene bridging group or C₁₋₃haloalkylene bridging group, is a group of formula:

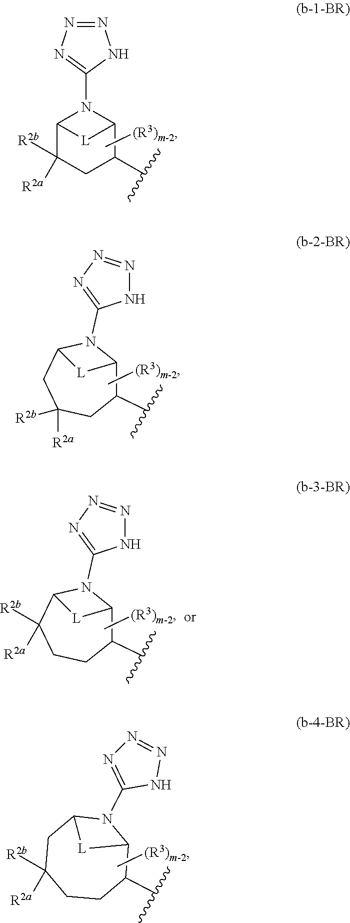

wherein L is a C₁₋₃ alkylene bridging group or C₁₋₃ haloalkylene bridging group.

Exemplary Embodiment 68. The compound of Exemplary Embodiment 67, or a pharmaceutically acceptable salt or tautomer thereof, wherein Ring B is a group of formula:

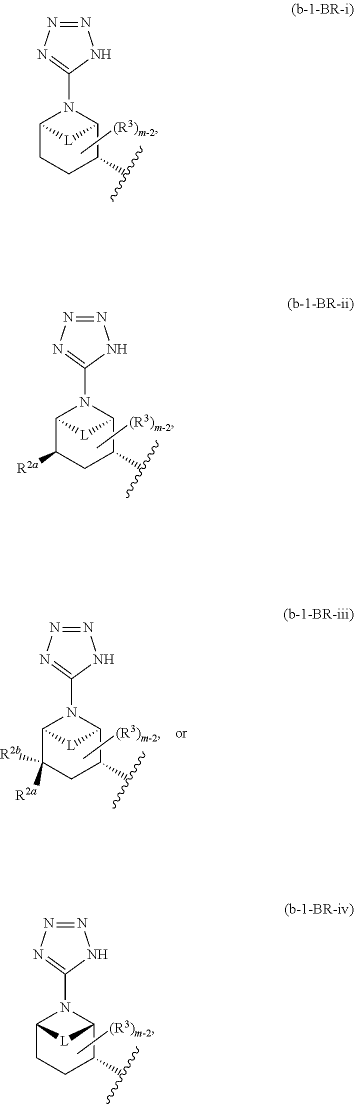

wherein each instance of R^{2a} and R² is independently halo, C₁₋₆ alkyl, C₁₋₆ haloalkyl, C₃-C₄ carbocyclyl, or 3-4 membered heterocyclyl.

Exemplary Embodiment 69. The compound of any one of Exemplary Embodiments 1-50, or a pharmaceutically acceptable salt or tautomer thereof, wherein Ring B is a group of formula:

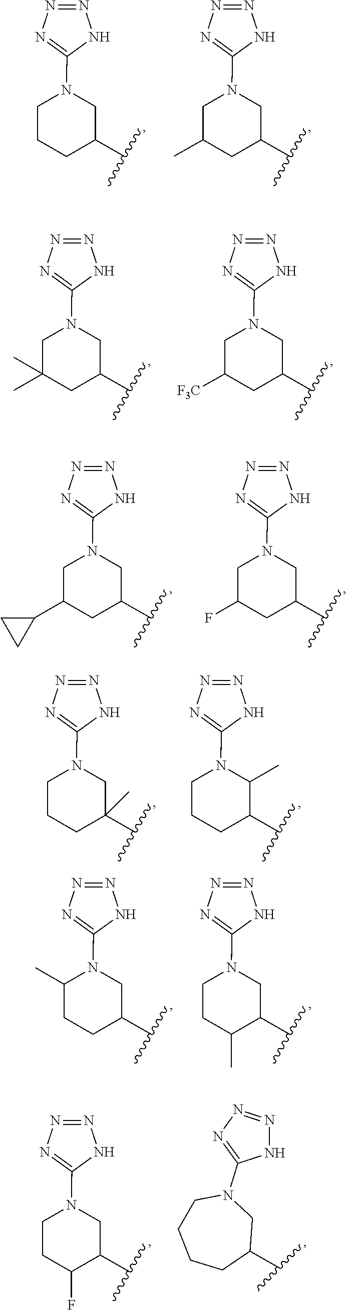

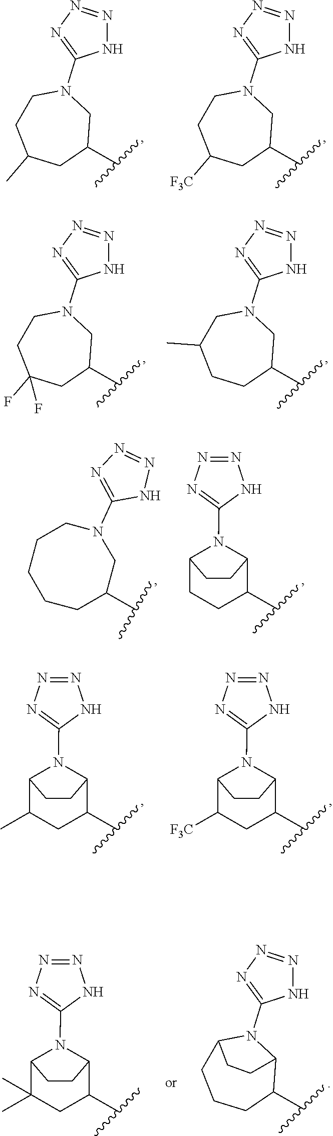

Exemplary Embodiment 70. The compound of any one of Exemplary Embodiments 1-50, or a pharmaceutically acceptable salt or tautomer thereof, wherein Ring B is a group of formula:

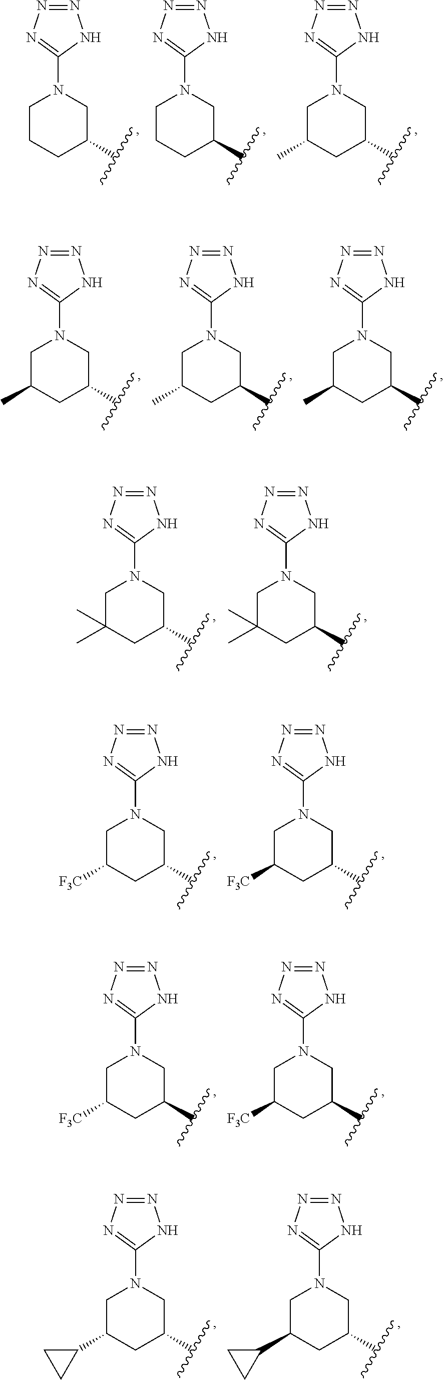

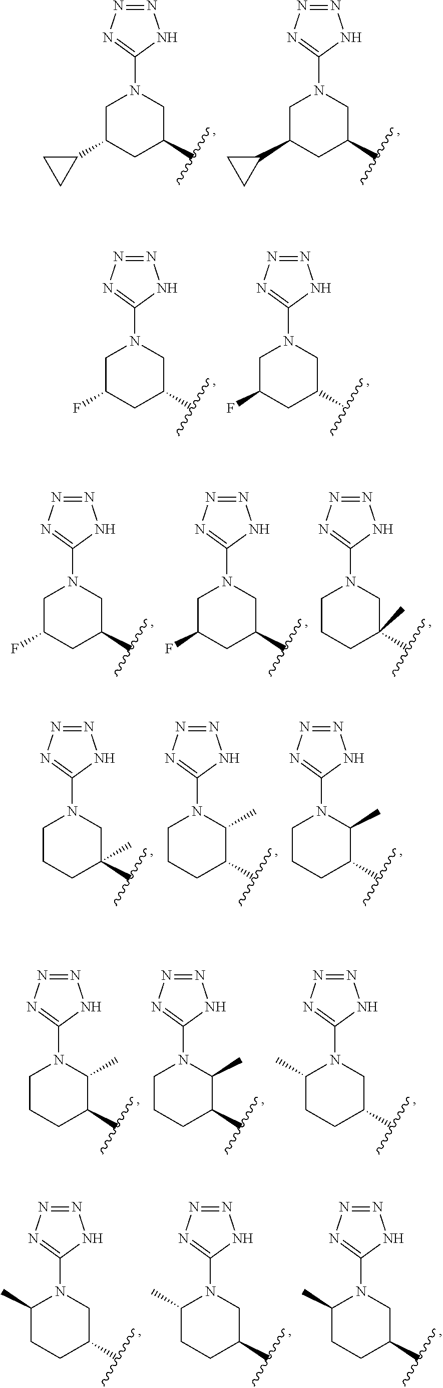

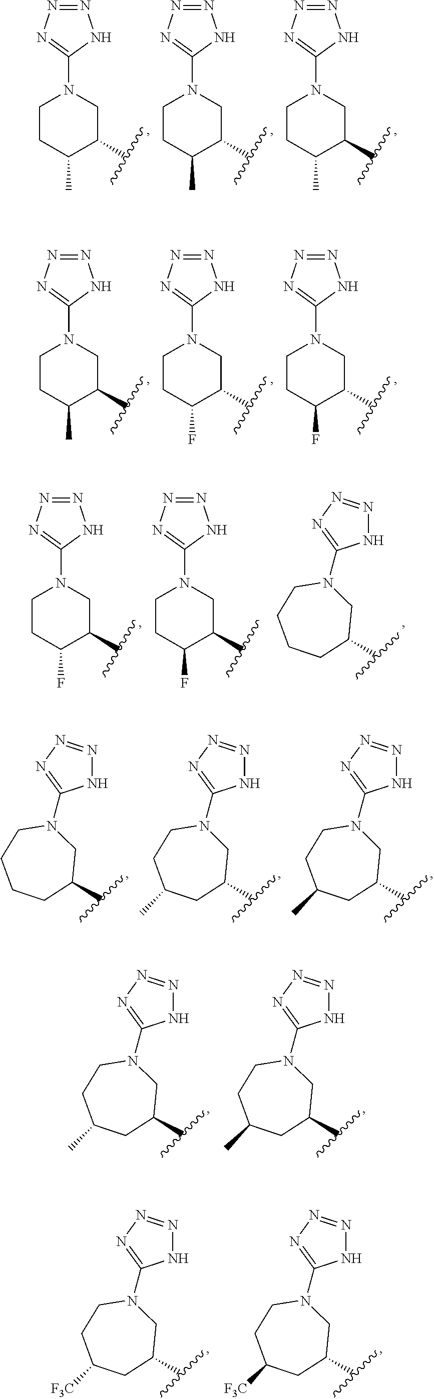

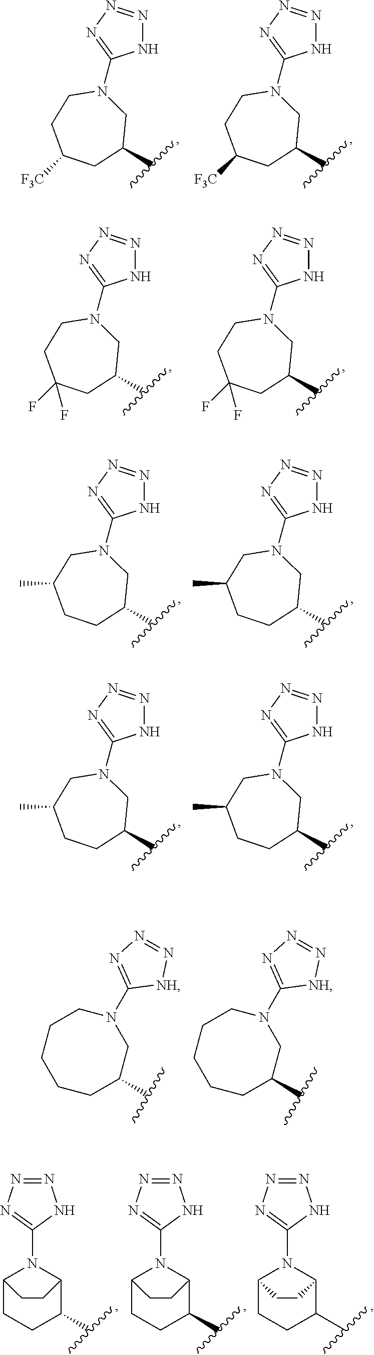

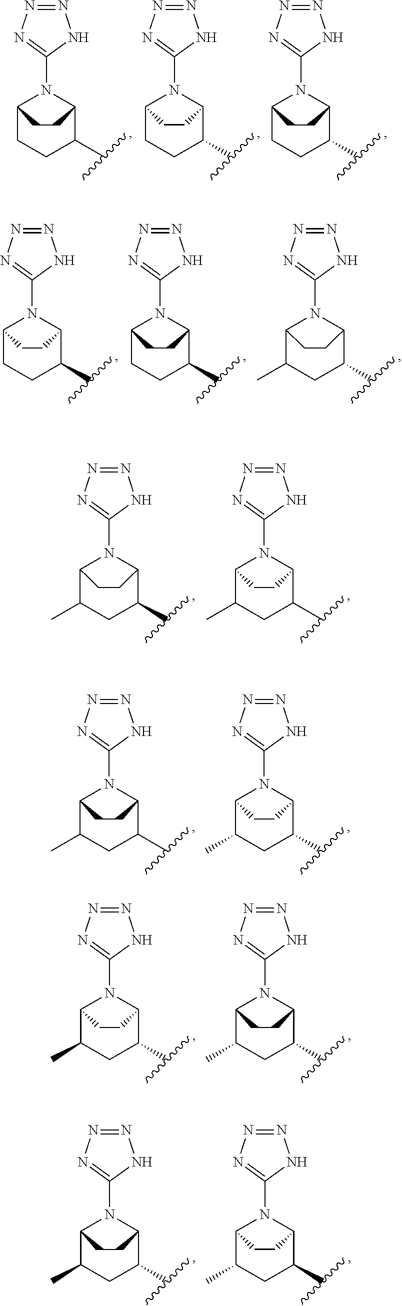

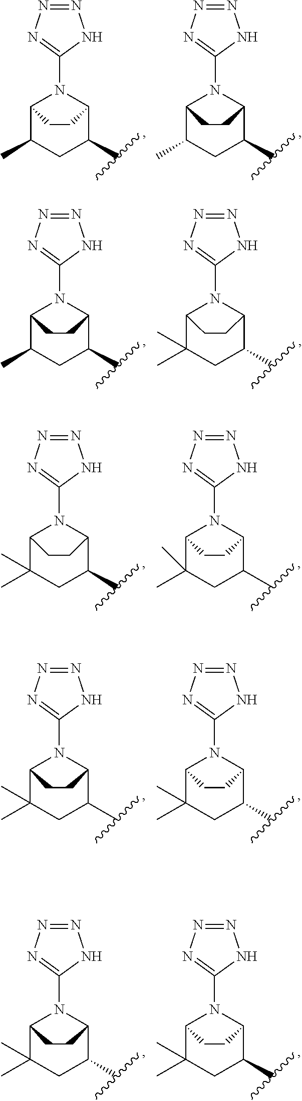

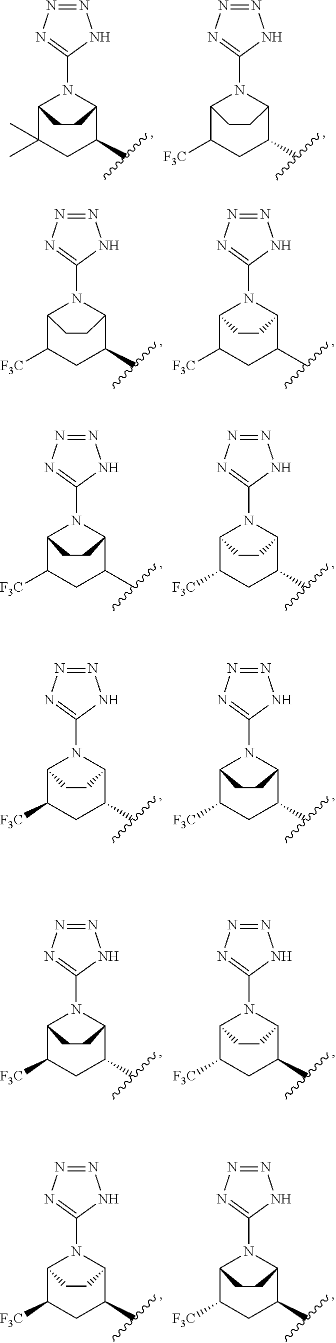

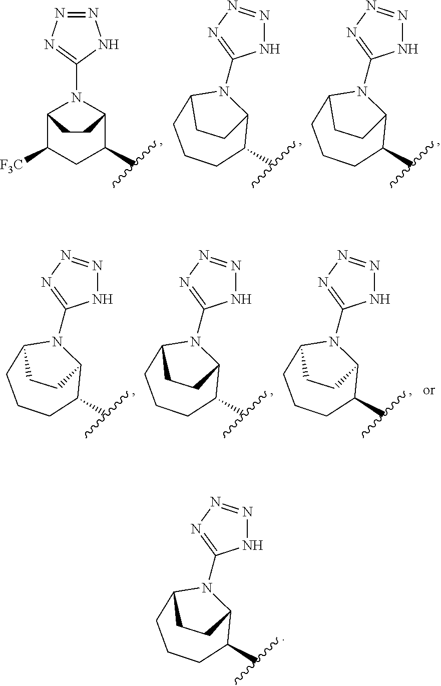

Exemplary Embodiment 71. The compound of any one of the preceding claims, wherein the compound is selected from the compounds described in Table 1 or Table 2, or a pharmaceutically acceptable salt or tautomer thereof.

Exemplary Embodiment 72. A pharmaceutical composition comprising the compound of any one of Exemplary Embodiments 1-71, or a pharmaceutically acceptable salt or tautomer thereof, and one or more pharmaceutically acceptable carriers.

Exemplary Embodiment 73. A method of modulating NLRP3 activity, the method comprising administering to the subject a compound of any one of Exemplary Embodiments 1-71, or a pharmaceutically acceptable salt or tautomer thereof, or a pharmaceutical composition of Exemplary Embodiment 72.

Exemplary Embodiment 74. A method of treating or preventing a disease or disorder, the method comprising administering to the subject a compound of any one of Exemplary Embodiments 1-71, or a pharmaceutically acceptable salt or tautomer thereof, or a pharmaceutical composition of Exemplary Embodiment 72.

Exemplary Embodiment 75. The compound of any one of Exemplary Embodiments 1-71, or a pharmaceutically acceptable salt or tautomer thereof, or a pharmaceutical composition of Exemplary Embodiment 72, for use in treating or preventing a disease or disorder.

Exemplary Embodiment 76. Use of the compound of any one of Exemplary Embodiments 1-71, or a pharmaceutically acceptable salt or tautomer thereof, in the manufacture of a medicament, for the treatment or prevention of a disease or disorder.

Exemplary Embodiment 77. Use of the compound of any one of Exemplary Embodiments 1-71, or a pharmaceutically acceptable salt or tautomer thereof, for the treatment or prevention of a disease or disorder.

Exemplary Embodiment 78. The method, compound, or use of any one of Exemplary Embodiments 73-77, wherein the disease or disorder is an NLRP3-related disease or disorder.

Exemplary Embodiment 79. The method, compound, or use of any one of Exemplary Embodiments 73-78, wherein the subject is a human.

Exemplary Embodiment 80. The method, compound, or use of any one of Exemplary Embodiments 73-79, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

Exemplary Embodiment 81. The method, compound, or use of Exemplary Embodiment 80, wherein the disease or disorder of the central nervous system is dementia, Alzheimer's disease (“AD”) epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), Huntington's disease (“HD”), or spinal cord injury.

Exemplary Embodiment 82. The method, compound, or use of Exemplary Embodiment 80, wherein the primary neurological disease of the muscle is dystrophies or spinal muscular atrophy.

Exemplary Embodiment 83. The method, compound, or use of Exemplary Embodiment 80, wherein the inflammatory disorder is gout or anemia of inflammation.

Exemplary Embodiment 84. The method, compound, or use of Exemplary Embodiment 80, wherein the autoimmune disease is ulcerative colitis.

Exemplary Embodiment 85. The method, compound, or use of Exemplary Embodiment 80, wherein the cancer is skin cancer or colon cancer.

Exemplary Embodiment 86. The method, compound, or use of Exemplary Embodiment 80, wherein the infection is a neuro-infection.

Exemplary Embodiment 87. The method, compound, or use of Exemplary Embodiment 80, wherein the metabolic disease is diabetes.

Exemplary Embodiment 88. The method, compound, or use of Exemplary Embodiment 80, wherein the cardiovascular disease is stroke.

Exemplary Embodiment 89. The method, compound, or use of Exemplary Embodiment 80, wherein the respiratory disease is asthma or chronic obstructive pulmonary disease.

Exemplary Embodiment 90. The method, compound, or use of Exemplary Embodiment 80, wherein the kidney disease is acute kidney disease, a chronic kidney disease, or a rare kidney disease.

Exemplary Embodiment 91. The method, compound, or use of Exemplary Embodiment 80, wherein the liver disease is nonalcoholic fatty liver disease (NAFLD) or non-alcoholic steatohepatitis (NASH).

Exemplary Embodiment 92. The method, compound, or use of Exemplary Embodiment 80, wherein the ocular disease is optic neuritis or macular degeneration.

Exemplary Embodiment 93. The method, compound, or use of Exemplary Embodiment 80, wherein the skin disease is psoriasis, hidradenitis suppurativa (HS), or atopic dermatitis.

Exemplary Embodiment 94. The method, compound, or use of Exemplary Embodiment 80, wherein the rheumatic disease is osteoarthritis, dermatomyositis, Still's disease, or juvenile idiopathic arthritis.

Exemplary Embodiment 95. The method, compound, or use of Exemplary Embodiment 80, wherein the psychological disease is a neuropsychiatric condition selected from the group consisting of depression, major depressive disorder, and refractory depression.

Exemplary Embodiment 96. The method, compound, or use of Exemplary Embodiment 80, wherein the pain is pain management addiction, osteoarthritis pain, or allodynia.

Exemplary Embodiment 97. The method, compound, or use of Exemplary Embodiment 80, wherein the NLRP3-related disease in a subject that has been determined to carry a germline or somatic non-silent mutation in NLRP3 is cryopyrin-associated autoinflammatory syndrome.

Exemplary Embodiment 98. The method, compound, or use of Exemplary Embodiment 80, wherein the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), spinal cord injury, dystrophies, neuro-infections, pain management addiction, neuropsychiatric conditions (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

Exemplary Embodiment 99. A process for preparing a compound of Formula (I-B) of any one of the preceding Exemplary Embodiments, or a salt or tautomer thereof, wherein the compound is synthesized according to General Schemes A or B.

### EXEMPLIFICATION

In order that this disclosure may be more fully understood, the following Examples are set forth. It should be understood that these examples are for illustrative purposes only and are not to be construed as limiting this disclosure in any manner.

### Analytical Methods

Nuclear magnetic resonance (NMR) spectra were recorded at 400 MHz as stated and at 300.3 K unless otherwise stated; the chemical shifts (6) are reported in parts per million (ppm). Spectra were recorded using a Bruker Avance 400 instrument with 8, 16 or 32 scans. Typical NMR solvents include deuterated dimethylsulfoxide (DMSO-d6) and deuterated methanol (CD₃OD).

Liquid Chromatography-Mass Spectrometry (LCMS) chromatograms and spectra were recorded using a Shimadzu LCMS-2020. Injection volumes were 0.7-8.0 μl and the flow rates were typically 0.8 or 1.2 mL/min. Detection methods were diode array (DAD) or evaporative light scattering (ELSD) as well as positive ion electrospray ionization. MS range was 100-1000 Da. Mobile phases of water and/or acetonitrile (MeCN) may contain a modifier (typically 0.01-0.04%) such as trifluoroacetic acid (TFA), formic acid (FA), or ammonium carbonate. ESI or ES=electrospray ionization; m/z=mass/charge; RT=retention time (minutes).

Purification/Separation Methods. The Synthetic methods describe purification and/or separation chromatographic methods which have been employed in the purification and/or isolation of the exemplified compounds. RT=retention time (minutes); Prep=Preparative High-performance liquid chromatography.

Compounds were numbered following the below numbering system, where R^{2a} and R^{2b} are not hydrogen.

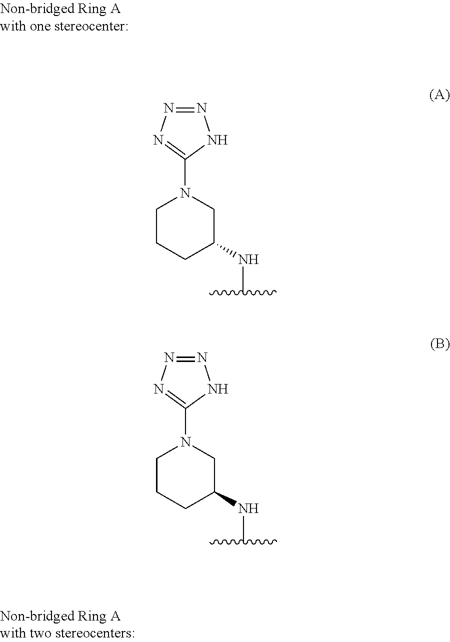 [SMILES: *N[C@@H]1CCCN(c2nnn[nH]2)C1 ; *N[C@H]1CCCN(c2nnn[nH]2)C1]

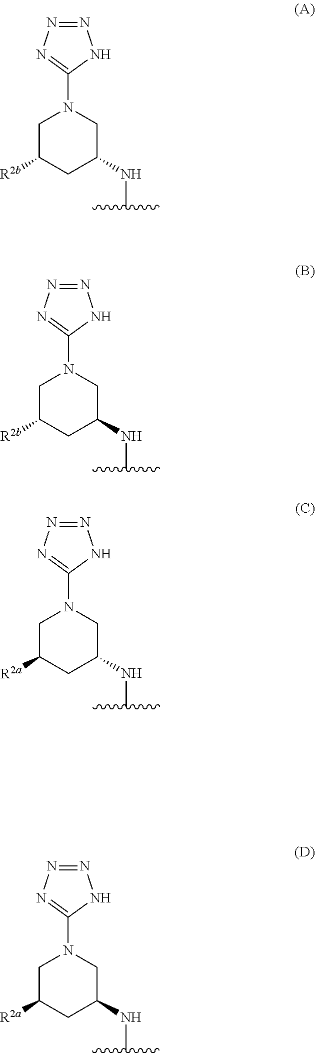

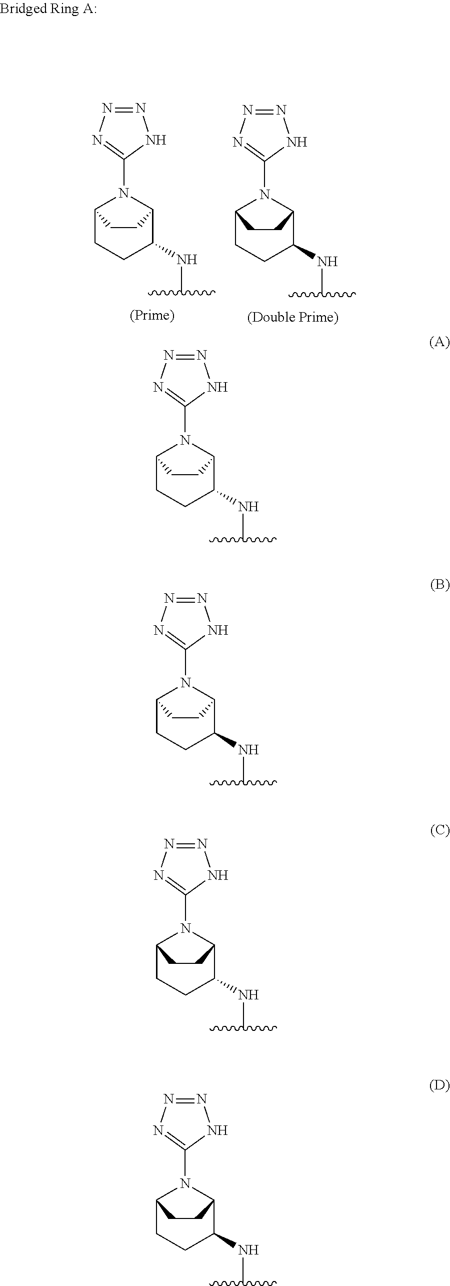 [SMILES: *N[C@@H]1CC[C@@H]2CC[C@H]1N2c1nnn[nH]1 ; *N[C@H]1CC[C@H]2CC[C@@H]1N2c1nnn[nH]1 ; *N[C@@H]1CC[C@@H]2CC[C@H]1N2c1nnn[nH]1 ; *N[C@H]1CC[C@@H]2CC[C@H]1N2c1nnn[nH]1 ; *N[C@@H]1CC[C@H]2CC[C@@H]1N2c1nnn[nH]1 ; *N[C@H]1CC[C@H]2CC[C@@H]1N2c1nnn[nH]1]

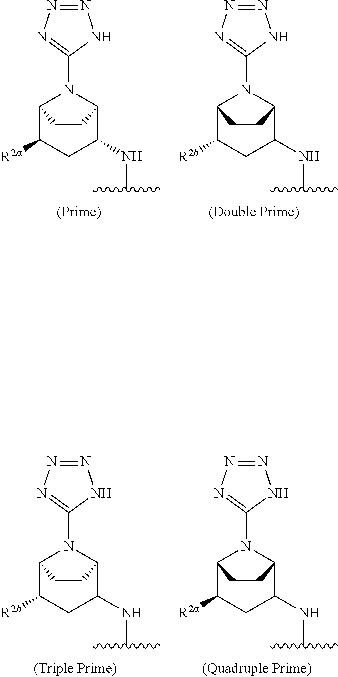

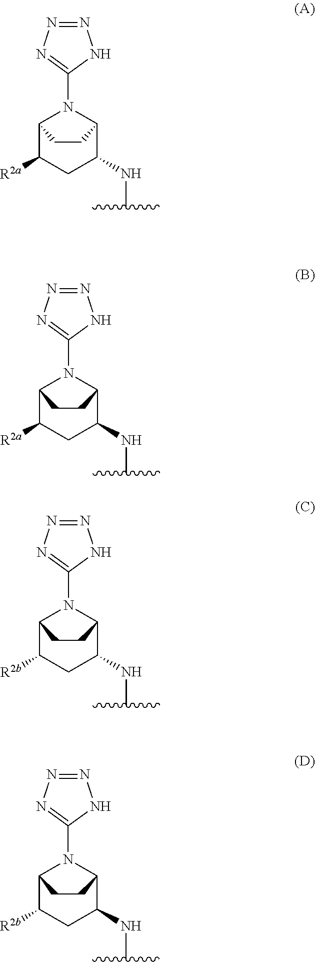

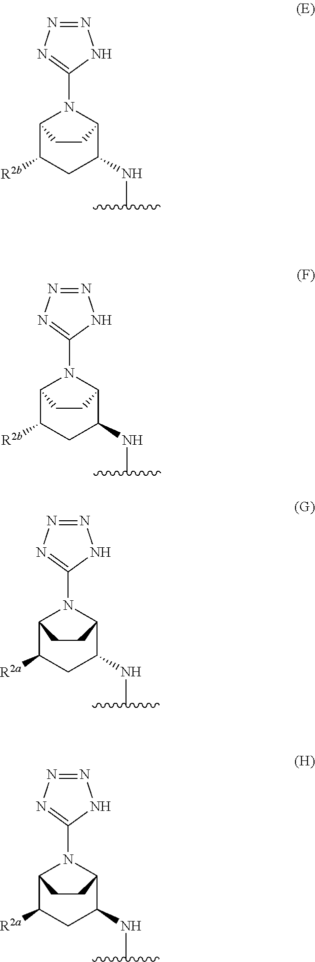

The Asterix (*) next to the Compound Number (#) signifies that arbitrary stereochemistry has been assigned. Future tense (“may be” prepared/synthesized) language signify examples to be conducted.

### SYNTHETIC EXAMPLES

### Example 1. (R)-3-(1-(1H-tetrazol-5-yl)piperidin-3-yl)-1-(4-chlorophenyl)-1-methylurea (Compound 2A) and (S)-3-(1-(1H-tetrazol-5-yl)piperidin-3-yl)-1-(4-chlorophenyl)-1-methylurea (Compound 2B)

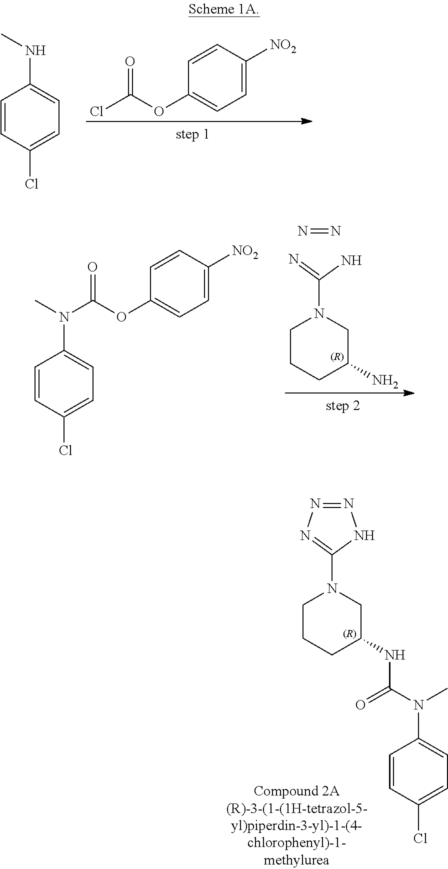

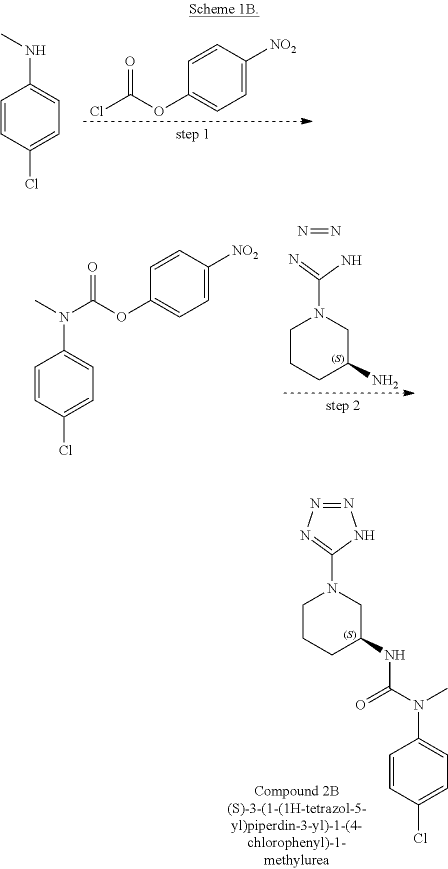

Example 1 follows Protocol B.

Step 1: To a stirred solution of 4-chloro-N-methylaniline (“aniline (iv) reagent”) (250 mg, 1.76 mmol, 1 equiv) and pyridine (419 mg, 5.29 mmol, 3 equiv) in dichloromethane (DCM) (5 mL) was added 4-nitrophenyl carbonochloridate (427 mg, 2.11 mmol, 1.2 equiv) dropwise at 0° C. The resulting mixture was stirred for 2 hours at room temperature, and the reaction was monitored by LCMS. Upon completion, the reaction was quenched by the addition of water (5 mL) at room temperature, and the resulting mixture was extracted with ethyl acetate (EtOAc) (2×15 mL). The combined organic layers were washed with brine (1×10 mL) and dried over anhydrous Na₂SO₄. After filtration, the filtrate was concentrated under reduced pressure to provide a residue, which was purified by silica gel column chromatography, eluting with petroleum ether/ethyl acetate (1:1), to provide 4-nitrophenyl N-(4-chlorophenyl)-N-methylcarbamate (400 mg, 74% yield). LCMS: (ES, m/z): RT=1.00 min, m/z=307.5[M+H]⁺.

Step 2: Into a 20 mL vial was added 4-nitrophenyl N-(4-chlorophenyl)-N-methylcarbamate (200 mg, 0.65 mmol, 1 equiv), potassium methaneperoxoate (454 mg, 3.26 mmol, 5 equiv), (3R)-1-(1H-1,2,3,4-tetrazol-5-yl)piperidin-3-amine (“amine (i) reagent”) (329 mg, 1.95 mmol, 3 equiv), and dimethylformamide (DMF) (4 mL) at 130° C. The reaction mixture was irradiated with microwave radiation for 1 hour at 130° C., and the reaction was monitored by LCMS. The resulting mixture was filtered, the filter cake was washed with MeOH (3×10 mL). The filtrate was concentrated under reduced pressure. The residue was purified by reverse flash chromatography (C18 silica gel; mobile phase: acetonitrile in water, 0% to 100% gradient in 20 min; detector: UV 254 nm) to provide a crude product (40 mg), which was further purified by Prep HPLC (YMC-Actus Triart C18 ExRS, 30*150 mm, 5 μm; Mobile Phase A: water (10 mmol/L (NH₄)HCO₃), Mobile Phase B: acetonitrile; Flow rate: 60 mL/min; Gradient: 5% B to 25% B in 10 min, 25% B; Wave Length: 254 nm; RT(min): 9.0) to provide Compound 2A (5.9 mg). LCMS: (ES, m/z): RT=1.33 min, m/z=336.0[M+H]⁺. ¹H NMR (400 MHz, DMSO-d₆) δ 7.45-7.34 (m, 2H), 7.34-7.22 (m, 2H), 6.09 (d, J=7.8 Hz, 1H), 3.80-3.57 (m, 3H), 3.15 (s, 3H), 2.84-2.64 (m, 2H), 1.83-1.62 (m, 2H), 1.61-1.34 (m, 2H).

Compound 2B may be prepared according to this Example using 4-chloro-N-methylaniline as the aniline (iv) reagent and (3S)-1-(1H-1,2,3,4-tetrazol-5-yl)piperidin-3-amine as the amine (i) reagent.

### Example 2. 1-(4-chlorophenyl)-1-methyl-3-(5-methyl-1-(1H-tetrazol-5-yl)azepan-3-yl)urea (Compound 4, Rac-4); 1-(4-chlorophenyl)-1-methyl-3-((3R,5R)-5-methyl-1-(1H-tetrazol-5-yl)azepan-3-yl)urea (Compound 4A*); 1-(4-chlorophenyl)-1-methyl-3-((3S,5R)-5-methyl-1-(1H-tetrazol-5-yl)azepan-3-yl)urea (Compound 4B*); 1-(4-chlorophenyl)-1-methyl-3-((3R,5S)-5-methyl-1-(1H-tetrazol-5-yl)azepan-3-yl)urea (Compound 4C*); and 1-(4-chlorophenyl)-1-methyl-3-((3S,5S)-5-methyl-1-(1H-tetrazol-5-yl)azepan-3-yl)urea (Compound 4D*)

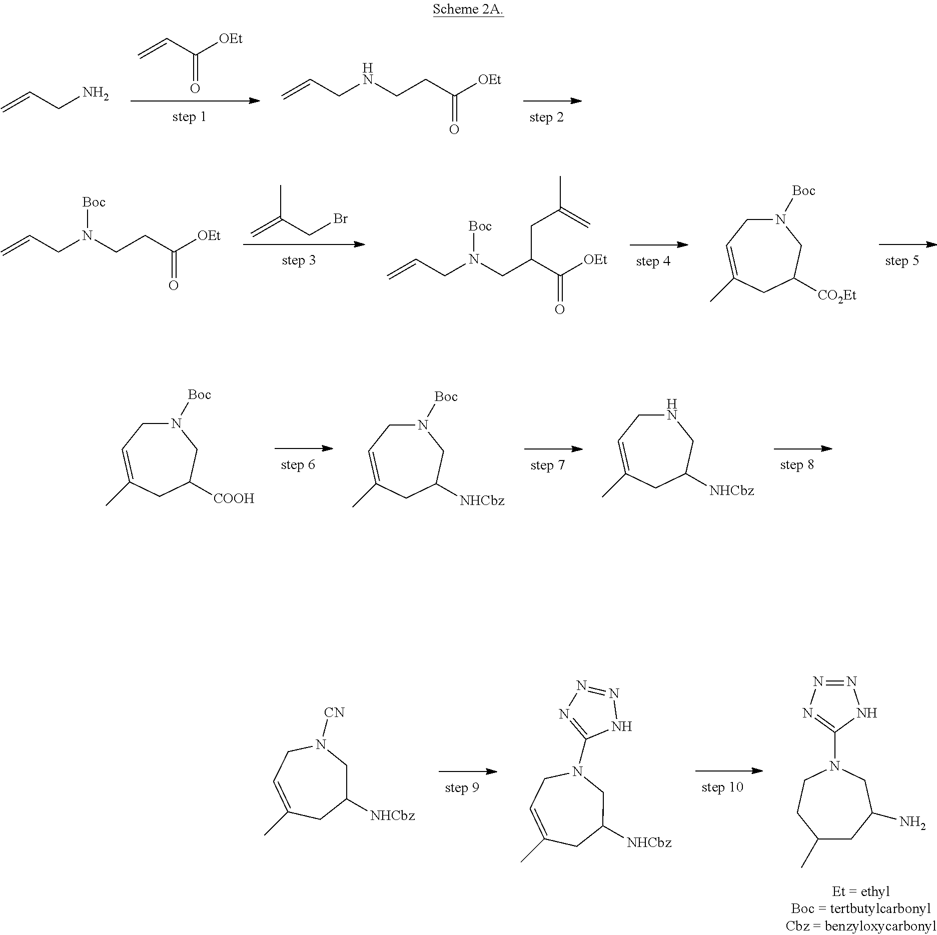

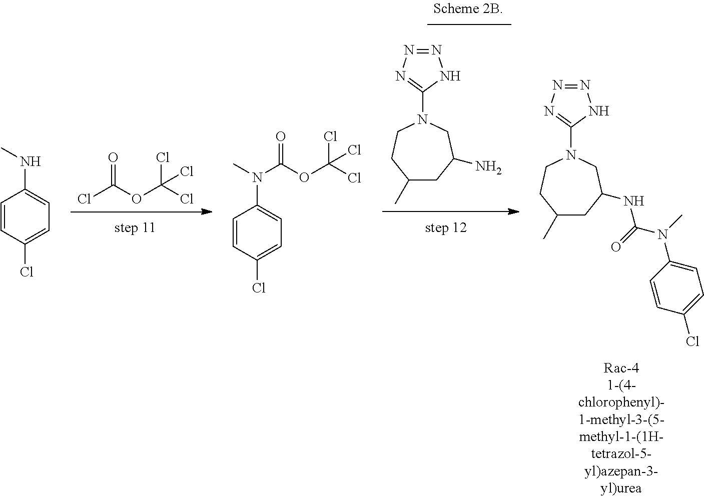

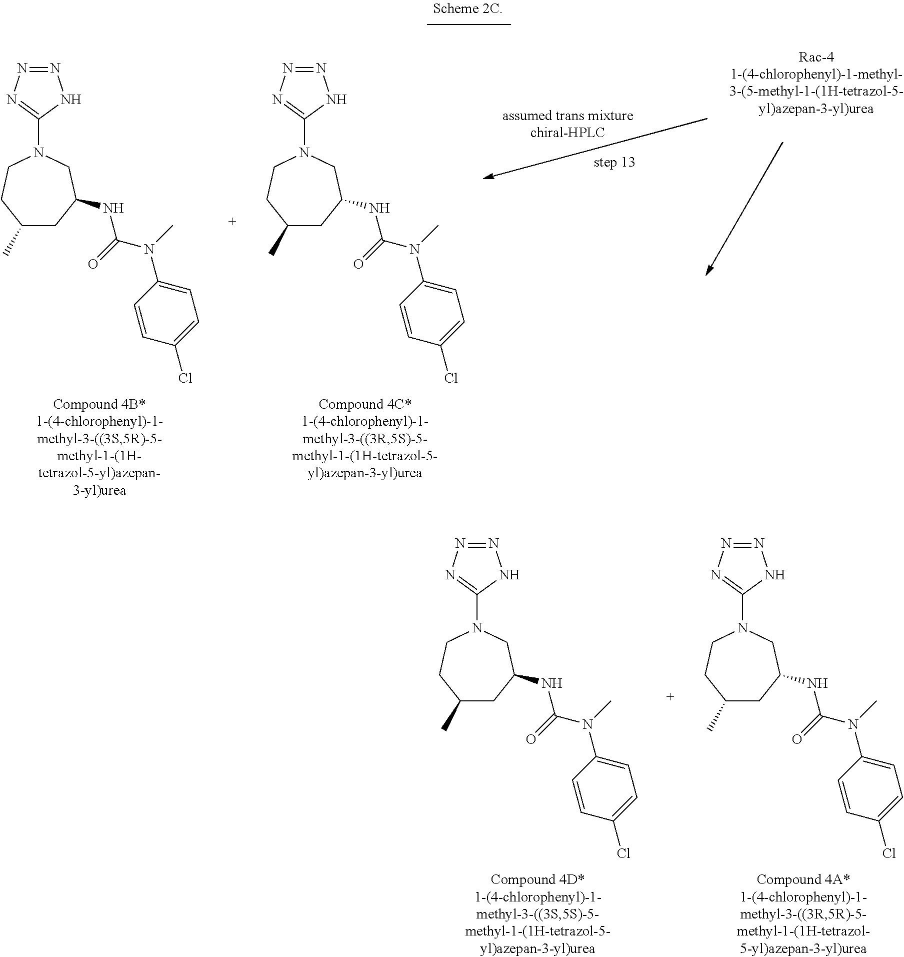 [SMILES: C[C@@H]1CCN(c2nnn[nH]2)C[C@@H](NC(=O)N(C)c2ccc(Cl)cc2)C1 ; C[C@H]1CCN(c2nnn[nH]2)C[C@H](NC(=O)N(C)c2ccc(Cl)cc2)C1 ; C[C@H]1CCN(c2nnn[nH]2)C[C@@H](NC(=O)N(C)c2ccc(Cl)cc2)C1 ; C[C@@H]1CCN(c2nnn[nH]2)C[C@H](NC(=O)N(C)c2ccc(Cl)cc2)C1]

Example 2 follows Protocol B.

Step 1: Into a 1000 mL round-bottom flask was added allylamine hydrochloride (25.0 g, 267 mmol, 1 equiv), ethanol (EtOH) (400 mL), ethyl acrylate (32.1 g, 320 mmol, 1.2 equiv) and 1,8-diazabicyclo[5.4.0]undec-7-ene (DBU) (81.4 g, 534 mmol, 2 equiv) at room temperature. The resulting mixture was stirred for 3 hours at room temperature under nitrogen atmosphere, quenched by the addition of water (100 mL) at room temperature, and then concentrated under reduced pressure. The aqueous layer was extracted with ethyl acetate (EtOAc) (3×500 mL), and the organic layer was dried over anhydrous Na₂SO₄, filtered, and the filtrate concentrated under reduced pressure to provide a crude product, ethyl 3-(prop-2-en-1-ylamino)propanoate (26.0 g, 62% yield). LCMS: (ES, m/z): RT=0.236 min, m/z=158[M+1]⁺.

Step 2: Into a 500 mL 3-necked round-bottom flask was added ethyl 3-(prop-2-en-1-ylamino)propanoate (25.0 g, 159 mmol, 1 equiv), di-tert-butyl dicarbonate (69.4 g, 318 mmol, 2 equiv), and dichloromethane (DCM) (300 mL) at 0° C. The resulting mixture was stirred for 2 hours at room temperature, then the reaction was quenched by the addition of water/ice (200 mL) at 0° C. The aqueous layer was extracted with ethyl acetate (EtOAc) (3×500 mL), and the organic layer was dried over anhydrous Na₂SO₄, concentrated under reduced pressure to provide a crude residue, which was purified by silica gel column chromatography, eluting with petroleum ether/ethyl acetate (12:1), to provide ethyl 3-[(tert-butoxycarbonyl)(prop-2-en-1-yl)amino]propanoate (23.0 g, 56% yield). LCMS: (ES, m/z): RT=0.993 min, m/z=258[M+1]⁺.

Step 3: To a stirred solution of ethyl 3-[(tert-butoxycarbonyl)(prop-2-en-1-yl)amino]propanoate (20.0 g, 77.7 mmol, 1 equiv) in tetrahydrofuran (THF) (300 mL) was added lithium hexamethyldisilazide (LiHMDS) (15.6 g, 93.3 mmol, 1.2 equiv) dropwise at −78° C. under nitrogen atmosphere, and the resulting mixture was stirred for 30 min at −78° C. under nitrogen atmosphere. 3-Bromo-2-methylprop-1-ene (20.8 g, 155 mmol, 2 equiv) was then added dropwise at −78° C. under nitrogen atmosphere, and the resulting mixture was stirred for 1 hour at 0° C. under nitrogen atmosphere. The reaction was quenched by the addition of sat. NH₄Cl (aq.) (500 mL) at 0° C., the aqueous layer was then extracted with ethyl acetate (EtOAc) (3×500 mL), the organic layer was dried over anhydrous Na₂SO₄, filtered, and the filtrate concentrated under reduced pressure to provide a crude residue, which was purified by silica gel column chromatography, eluting with petroleum ether/ethyl acetate (12:1), to provide ethyl 2-{[(tert-butoxycarbonyl)(prop-2-en-1-yl)amino]methyl}pent-4-enoate (15.0 g, 65% yield). LCMS: (ES, m/z): RT=1.097 min, m/z=312[M+1]⁺.

Step 4: Into a 5000 mL 3-necked round-bottom flask was added ethyl 2-{[(tert-butoxycarbonyl)(prop-2-en-1-yl)amino]methyl}-4-methylpent-4-enoate (40.0 g, 128 mmol, 1 equiv), tetrahydrofuran (THF) (4000 mL), and dichloro[1,3-bis(2,4,6-trimethylphenyl)-2-imidazolidinylidene](benzylidene)(tricyclohexylphosphine)ruthenium(II) (Grubbs 2nd generation catalyst) (16.4 g, 19.3 mmol, 0.15 equiv) at room temperature. The resulting mixture was stirred for 2 hours at 55° C. under nitrogen atmosphere, then quenched by the addition of water (500 mL). The aqueous layer was then extracted with ethyl acetate (EtOAc) (3×300 mL), and the organic layer was dried over anhydrous Na₂SO₄, filtered, and the filtrate concentrated under reduced pressure to provide a residue, which was purified by silica gel column chromatography, eluting with petroleum ether/ethyl acetate (12:1), to provide 1-tert-butyl 3-ethyl 5-methyl-2,3,4,7-tetrahydroazepine-1,3-dicarboxylate) (30.0 g, 82% yield).

Step 5: Into a 500 mL round-bottom flask was added 1-tert-butyl 3-ethyl 5-methyl-2,3,4,7-tetrahydroazepine-1,3-dicarboxylate (20.0 g, 70.6 mmol, 1 equiv), methanol (MeOH) (100 mL), NaOH (14.1 g, 353 mmol, 5 equiv), and water (100 mL) at room temperature. The resulting mixture was stirred for 2 hours at room temperature. The mixture was then acidified to pH 6 with HCl (aq)(2M) at 0° C., the aqueous layer was extracted with ethyl acetate (EtOAc) (3×200 mL), and the organic layer was dried over anhydrous Na₂SO₄ and filtered. The filtrate was then concentrated under reduced pressure to provide crude 1-tert-butyl 3-ethyl 5-methyl-2,3,4,7-tetrahydroazepine-1,3-dicarboxylate.

Step 6: Into a 500 mL 3-necked round-bottom flask was added 1-(tert-butoxycarbonyl)-5-methyl-2,3,4,7-tetrahydroazepine-3-carboxylic acid (20.0 g, 78.3 mmol, 1 equiv), toluene (250 mL), benzyl alcohol (25.4 g, 235 mmol, 3 equiv), diphenylphosphoryl azide (DPPA) (64.7 g, 235 mmol, 3 equiv) and triethylamine (23.8 g, 235 mmol, 3 equiv) at room temperature. The resulting mixture was stirred for 3 hours at 100° C. under nitrogen atmosphere, concentrated under reduced pressure, added water (500 mL) and the aqueous layer was extracted with ethyl acetate (3×500 mL), and the organic layer was dried over anhydrous Na₂SO₄, filtered, and the filtrate concentrated under reduced pressure to provide a residue, which was purified by reverse flash chromatography (C18 silica column; mobile phase: acetonitrile in water, 50% to 60% gradient in 10 min; detector: UV 254 nm) to provide tert-butyl 3-{[(benzyloxy)carbonyl]amino}-5-methyl-2,3,4,7-tetrahydroazepine-1-carboxylate (12 g, 43% yield). LCMS: (ES, m/z): RT=1.065 min, m/z=361[M+1]⁺.

Step 7: Into a 100 mL round-bottom flask was added tert-butyl 3-{[(benzyloxy)carbonyl]amino}-5-methyl-2,3,4,7-tetrahydroazepine-1-carboxylate (10.0 g, 27.8 mmol, 1 equiv), dichloromethane (DCM) (25 mL) and trifluoroacetic acid (TFA) (5 mL) at room temperature. The resulting mixture was stirred for 1 hour at room temperature. The resulting mixture was then concentrated under reduced pressure to provide crude benzyl N-(5-methyl-2,3,4,7-tetrahydro-1H-azepin-3-yl)carbamate (8.0 g). LCMS: (ES, m/z): RT=0.563 min, m/z=261[M+1]⁺.

Step 8: Into a 250 mL round-bottom flask was added benzyl N-(5-methyl-2,3,4,7-tetrahydro-1H-azepin-3-yl)carbamate (8.0 g, 30.7 mmol, 1 equiv), acetonitrile (50 mL), K₂CO₃ (12.8 g, 92.2 mmol, 3 equiv) and BrCN (3.91 g, 36.9 mmol, 1.2 equiv) at room temperature. The resulting mixture was stirred for 1 hour at room temperature under nitrogen atmosphere and added water (100 mL). The aqueous layer was then extracted with ethyl acetate (EtOAc) (3×200 mL), and the organic layer was concentrated under reduced pressure to provide a crude residue, which was purified by silica gel column chromatography, eluting with petroleum ether/ethyl acetate (10:1), to provide benzyl N-(1-cyano-5-methyl-2,3,4,7-tetrahydroazepin-3-yl)carbamate (7.0 g, 80% yield). LCMS: (ES, m/z): RT=0.837 min, m/z=286[M+1]⁺.

Step 9: Into a 50 mL round-bottom flask was added benzyl N-(1-cyano-5-methyl-2,3,4,7-tetrahydroazepin-3-yl)carbamate (5.0 g, 17.5 mmol, 1 equiv), NH₄Cl (2.81 g, 52.6 mmol, 3 equiv) and azidotrimethylsilane (6.06 g, 52.6 mmol, 3 equiv) at room temperature. The resulting mixture was stirred for 2 hours at 100° C. under nitrogen atmosphere. The mixture was allowed to cool down to room temperature, concentrated to provide a residue, which was purified by reverse flash chromatography (C18 silica column; mobile phase: acetonitrile in water, 40% to 60% gradient in 10 min; detector: UV 254 nm) to provide benzyl N-[5-methyl-1-(1H-1,2,3,4-tetrazol-5-yl)-2,3,4,7-tetrahydroazepin-3-yl]carbamate (4 g, 70% yield). LCMS: (ES, m/z): RT=0.773 min, m/z=329[M+1]⁺.

Step 10: Into a 250 mL round-bottom flask was added benzyl N-[5-methyl-1-(1H-1,2,3,4-tetrazol-5-yl)-2,3,4,7-tetrahydroazepin-3-yl]carbamate (4.0 g, 12.2 mmol, 1 equiv), methanol (MeOH) (100 mL) and Pd/C (1.30 g, 12.2 mmol) at room temperature. The resulting mixture was stirred for overnight at 70° C. under hydrogen atmosphere. The resulting mixture was then filtered, the filter cake was washed with methanol (MeOH) (3×20 mL), and the filtrate was then concentrated under reduced pressure to provide the crude product, 5-methyl-1-(1H-1,2,3,4-tetrazol-5-yl)azepan-3-amine (“amine (i) reagent”) (2.5 g). LCMS: (ES, m/z): RT=0.101 min, 0.289, m/z=197[M+1]⁺.

Step 11: Into an 8 mL vial was added 4-chloro-N-methylaniline (“aniline (iv) reagent”) (250 mg, 1.76 mmol, 1 equiv), triethylamine (TEA) (536 mg, 5.29 mmol, 3 equiv) and dichloromethane (DCM) (3 mL). To the above mixture was added diphosgene (419 mg, 2.11 mmol, 1.2 equiv) at 0° C. The resulting mixture was stirred for additional 1 hour at 25° C., and the reaction was monitored by LCMS. Upon completion of the reaction, the resulting mixture was filtered, the filter cake was washed with ethyl acetate (EtOAc) (3×10 mL), and the filtrate was concentrated under reduced pressure to provide a residue, which was purified by Prep-TLC (petroleum ether/ethyl acetate 3:1) to provide trichloromethyl N-(4-chlorophenyl)-N-methylcarbamate (400 mg, 75% yield). LCMS: (ES, m/z): RT=1.09 min.

Step 12: Into a 40 mL vial was added 4-nitrophenyl N-(4-chlorophenyl)-N-methylcarbamate (200 mg, 0.652 mmol, 1 equiv), potassium methaneperoxoate (272 mg, 1.96 mmol, 3 equiv), 5-methyl-1-(1H-1,2,3,4-tetrazol-5-yl)azepan-3-amine (154 mg, 0.782 mmol, 1.2 equiv) and acetonitrile (6 mL). The resulting mixture was stirred for 12 hours at 80° C., and the reaction was monitored by LCMS. Upon completion, the resulting mixture was filtered, the filter cake was washed with methanol (MeOH) (3×10 mL), and the filtrate was concentrated under reduced pressure to provide a residue, which was purified by reverse flash chromatography (C18 silica gel; mobile phase: acetonitrile (MeCN) in water (0.1% NH₃·H₂O), 0% to 100% gradient in 20 min; detector, UV 254 nm) to provide the crude product 1-(4-chlorophenyl)-1-methyl-3-(5-methyl-1-(1H-tetrazol-5-yl)azepan-3-yl)urea (Compound 4, Rac-4) (300 mg), which was then further purified by Prep HPLC (XSelect CSH C18 OBD Column 30*150 mm 5 μm; Mobile Phase A: acetonitrile, Mobile Phase B: water (0.05% trifluoroacetic acid); Flow rate: 60 mL/min; Gradient: 34% B to 44% B in 10 min, 44% B; Wave Length: 254 nm; RT(min): 6.32-8.77) to provide a mixture of two trans isomers of 1-(4-chlorophenyl)-1-methyl-3-(5-methyl-1-(1H-tetrazol-5-yl)azepan-3-yl)urea (79 mg, purity=99.00%), and a mixture of two cis isomers of 1-(4-chlorophenyl)-1-methyl-3-(5-methyl-1-(1H-tetrazol-5-yl)azepan-3-yl)urea (13 mg, purity=98.00%), designation of trans and cis isolates arbitrarily assigned. Trans isomers: LCMS: (ES, m/z): RT=0.63 min, m/z=364.0[M+H]⁺. Cis isomers: LCMS: (ES, m/z): RT=0.61 min, m/z=364.0[M+H]⁺.

Step 13: The mixture of trans isomers of 1-(4-chlorophenyl)-1-methyl-3-(5-methyl-1-(1H-tetrazol-5-yl)azepan-3-yl)urea (79 mg) was purified by Prep-chiral HPLC (CHIRALPAK IG, 2*25 cm, 5 m; Mobile Phase A: hexanes (0.1% trifluoroacetic acid), Mobile Phase B: ethanol; Flow rate: 20 mL/min; Gradient: 15% B to 15% B in 21 min; Wave Length: 254/220 nm) to provide Compound 4B* (RT(min): 17.35, 26.0 mg) and Compound 4C* (RT(min): 19.44, 23.0 mg). Stereochemistry arbitrarily assigned.

Compound 4B*: LCMS: (ES, m/z): RT=0.65 min, m/z=364.0[M+H]⁺. ¹H NMR (400 MHz, DMSO-d₆) δ 7.41-7.32 (m, 2H), 7.28-7.20 (m, 2H), 5.93 (s, J=7.8 Hz, 1H), 4.11 (s, J=8.0, 4.3 Hz, 1H), 3.62 (s, J=18.7, 10.1, 4.8 Hz, 3H), 3.22 (s, J=13.9, 9.8, 4.3 Hz, 1H), 3.14 (s, 3H), 1.91-1.81 (m, 1H), 1.70 (s, J=15.4 Hz, 1H), 1.61 (s, J=14.2, 5.7, 2.2 Hz, 1H), 1.49 (s, J=14.4, 9.7, 4.7 Hz, 1H), 1.34 (s, J=13.9, 9.2, 4.6 Hz, 1H), 0.89 (s, J=6.8 Hz, 3H).

Compound 4C*: LCMS: (ES, m/z): RT=0.64 min, m/z=364.0[M+H]⁺. ¹H NMR (400 MHz, Methanol-d₄) δ 7.39-7.31 (m, 2H), 7.21-7.13 (m, 2H), 4.11 (s, J=4.1 Hz, 1H), 3.73 (s, J=15.0, 6.6 Hz, 1H), 3.68-3.50 (m, 2H), 3.26 (s, J=8.4, 4.6 Hz, 1H), 3.22 (s, 3H), 1.88-1.59 (m, 4H), 1.48 (s, J=13.7, 9.3, 4.2 Hz, 1H), 0.99 (s, J=6.4 Hz, 3H).

Step 14: The mixture of cis isomers (13 mg) of 1-(4-chlorophenyl)-1-methyl-3-(5-methyl-1-(1H-tetrazol-5-yl)azepan-3-yl)urea was purified by Prep-chiral HPLC (CHIRALPAK IG, 2*25 cm, 5 μm; Mobile Phase A: hexanes (0.1% trifluoroacetic acid), Mobile Phase B: ethanol; Flow rate: 20 mL/min; Gradient: 20% B to 20% B in 14 min; Wave Length: 254/220 nm) to provide Compound 4A* (RT(min): 12.72, 4.3 mg) and Compound 4D* (RT(min): 9.84, 5.4 mg). Stereochemistry arbitrarily assigned.

Compound 4A*: LCMS: (ES, m/z): RT=1.15 min, m/z=364.0[M+H]⁺. ¹H NMR (400 MHz, DMSO-d₆) δ 14.68 (s, 1H), 7.47-7.38 (m, 2H), 7.35-7.26 (m, 2H), 6.41 (s, J=7.8 Hz, 1H), 3.83 (s, J=12.9 Hz, 1H), 3.62 (s, J=14.2, 4.9 Hz, 1H), 3.56 (m, 2H), 3.48-3.37 (m, 2H), 3.17 (s, 2H), 1.76 (s, J=13.7 Hz, 1H), 1.65 (s, J=13.3 Hz, 1H), 1.52 (s, 1H), 1.45-1.31 (m, 1H), 1.24 (s, J=11.4 Hz, 1H), 0.91 (s, J=6.7 Hz, 3H).

Compound 4D*: LCMS: (ES, m/z): RT=0.73 min, m/z=364.0[M+H]⁺. ¹H NMR (400 MHz, DMSO-d₆) δ 14.69 (s, 1H), 7.46-7.38 (m, 2H), 7.35-7.26 (m, 2H), 6.42 (s, J=8.0 Hz, 1H), 3.87-3.79 (m, 1H), 3.62 (s, J=14.1, 4.9 Hz, 1H), 3.59 (m, 1H), 3.56-3.36 (m, 2H), 3.17 (s, 3H), 1.76 (s, J=14.1 Hz, 1H), 1.69-1.61 (m, 1H), 1.53 (s, 1H), 1.38 (s, J=15.4, 5.2 Hz, 1H), 1.30-1.17 (m, 1H), 0.91 (s, J=6.6 Hz, 3H).

### Example 3. 1-(4-chlorophenyl)-1-ethyl-3-((3R,5S)-5-methyl-1-(1H-tetrazol-5-yl)piperidin-3-yl)urea (Compound 6A), and Compounds 6B-6D

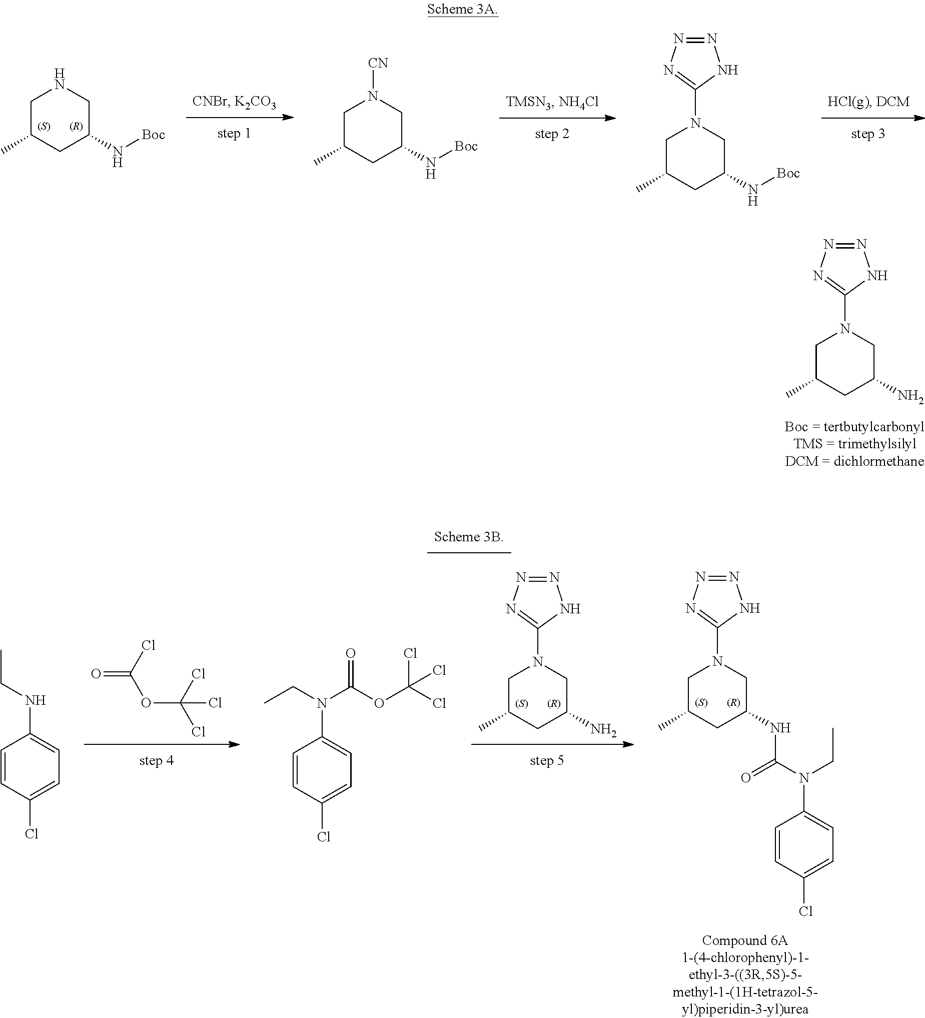

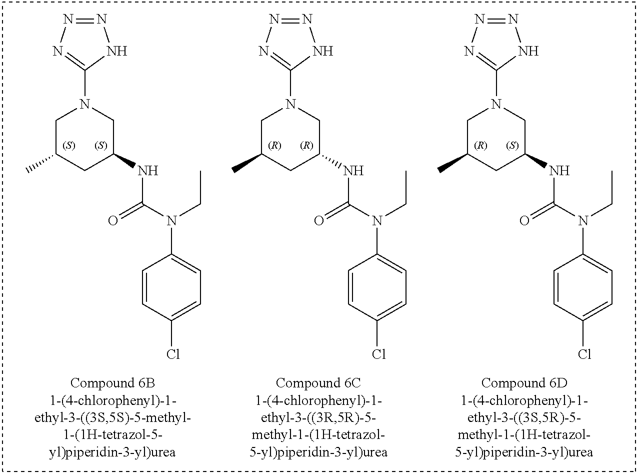

Example 3 follows Protocol B.

Step 1: Into a 50 mL round-bottom flask was added tert-butyl N-[(3R,5S)-5-methylpiperidin-3-yl]carbamate (1.5 g, 6.99 mmol, 1 equiv) and BrCN (1110 mg, 10.5 mmol, 1.5 equiv), acetonitrile (20 mL), and K₂CO₃ (2.9 g, 21 mmol, 3 equiv) at room temperature, and the resulting mixture was stirred for 2 hours at room temperature, and the reaction progress was monitored by LCMS. Upon completion of the reaction, the resulting mixture was filtered, and the filtrate concentrated under reduced pressure to provide tert-butyl-N-[(3R,5S)-1-cyano-5-methylpiperidin-3-yl]carbamate (1.6 g, purity=80%). LCMS: (ES, m/z): RT=0.603 min, m/z=240[M+1]⁺.

Step 2: Into a 50 mL round-bottom flask was added tert-butyl N-[(3R,5S)-1-cyano-5-methylpiperidin-3-yl]carbamate (1.6 g, 6.68 mmol, 1 equiv), NH₄Cl (1.08 g, 20.0 mmol, 3 equiv), trimethylsilyl azide (TMSN₃) (3120 mg, 13.4 mmol, 2 equiv), and dimethylformamide (DMF) (3 mL) at room temperature. The resulting mixture was stirred for 16 hours at 100° C., and the reaction progress was monitored by LCMS. Upon completion of the reaction, the residue was purified by reverse flash chromatography (C18 silica gel column; mobile phase: acetonitrile in water, 10% to 50% gradient in 10 min; detector, UV 254 nm) to provide tert-butyl N-[(3R,5S)-5-methyl-1-(1H-1,2,3,4-tetrazol-5-yl)piperidin-3-yl]carbamate (1.50 g, 80% yield). LCMS: (ES, m/z): RT=0.499 min, m/z=283[M+1]⁺.

Step 3: Into a 250 mL round-bottom flask was added tert-butyl N-[(3R,5S)-5-methyl-1-(1H-1,2,3,4-tetrazol-5-yl)piperidin-3-yl]carbamate (1.5 g, 5.313 mmol, 1 equiv) and dichloromethane (5 mL) at room temperature. The resulting mixture was stirred for 1 hour at room temperature under HCl (g) atmosphere, and then concentrated under reduced pressure to provide (3R,5S)-5-methyl-1-(1H-1,2,3,4-tetrazol-5-yl)piperidin-3-amine (“amine (i) reagent”) (1 g, HCl salt). LCMS: (ES, m/z): RT=0.151 min, m/z=183[M+1]⁺.

Step 4: A solution of 4-chloro-N-ethylaniline (“aniline (iv) reagent”) (1.00 g, 6.42 mmol, 1 equiv) in dichloromethane was treated with triethylamine (1.95 g, 19.3 mmol, 3 equiv) at 0° C. followed by the addition of diphosgene (1.40 g, 7.06 mmol, 1.1 equiv) dropwise at 0° C. The resulting mixture was stirred for 2 hours at room temperature, and the reaction progress was monitored by LCMS. Upon completion of the reaction, the reaction mixture was concentrated to provide a crude residue, which was purified by silica gel column chromatography, eluting with petroleum ether/ethyl acetate (5:1), to provide trichloromethyl N-(4-chlorophenyl)-N-ethylcarbamate (1.00 g, 49.1%). LCMS: (ES, m/z) RT=0.76 min, m/z=276 [M+H]⁺.

Step 5: Into a 25 mL round-bottom flask was added trichloromethyl N-(4-chlorophenyl)-N-ethylcarbamate (10.0 mg, 0.03 mmol, 1 equiv), (3R,5S)-5-methyl-1-(1H-1,2,3,4-tetrazol-5-yl)piperidin-3-amine (HCl salt, 5.75 mg, 0.03 mmol, 1 equiv), K₂CO₃ (13.1 mg, 0.09 mmol, 3 equiv), and dimethylformamide (1 mL) at room temperature, and the reaction progress was monitored by LCMS. Upon completion of the reaction, the reaction mixture was concentrated under reduced pressure, and the resulting residue was purified by reverse flash chromatography (C18 silica gel column; mobile phase: acetonitrile in water, 10% to 50% gradient in 400 min; detector: UV 254 nm) to provide a crude product (30 mg), which was further purified by Prep HPLC (YMC-Actus Triart C18 ExRS, 30*150 mm, 5 μm; Mobile Phase A: water (10 mmol/L (NH₄)HCO₃), Mobile Phase B: acetonitrile; Flow rate: 60 mL/min; Gradient: 15% B to 25% B in 12 min, 25% B; Wave Length: 254 nm; RT(min): 11.2) to provide Compound 6A (15.0 mg). LCMS: (ES, m/z) RT=0.87 min, m/z=364 [M+H]⁺; ¹H NMR (400 MHz, DMSO-d6) δ 7.49-7.42 (m, 2H), 7.28-7.20 (m, 2H), 5.77 (d, J=8.2 Hz, 1H), 3.85 (m, J=11.7 Hz, 1H), 3.79-3.54 (m, 4H), 2.46 (s, 1H), 2.26 (t, J=11.9 Hz, 1H), 1.78 (d, J=12.6 Hz, 1H), 1.71-1.63 (m, 1H), 1.10-0.96 (m, 4H), 0.87 (d, J=6.5 Hz, 3H).

Compound 6B may be prepared according to this Example 3 using 4-chloro-N-ethylaniline as the aniline (iv) reagent and (3S,5S)-5-methyl-1-(1H-1,2,3,4-tetrazol-5-yl)piperidin-3-amine as the amine (i) reagent.

Compound 6C may be prepared according to this Example 3 using 4-chloro-N-ethylaniline as the aniline (iv) reagent and (3R,5R)-5-methyl-1-(1H-1,2,3,4-tetrazol-5-yl)piperidin-3-amine as the amine (i) reagent.

Compound 6D may be prepared according to this Example 3 using 4-chloro-N-ethylaniline as the aniline (iv) reagent and (3S,5R)-5-methyl-1-(1H-1,2,3,4-tetrazol-5-yl)piperidin-3-amine as the amine (i) reagent.

### Example 4. 1-(4-(difluoromethoxy)phenyl)-1-methyl-3-((1R,4R,5S)-4-methyl-8-(1H-tetrazol-5-yl)-8-azabicyclo[3.2.1]octan-2-yl)urea (Compound 42′*), 1-(4-(difluoromethoxy)phenyl)-1-methyl-3-((1S,4S,5R)-4-methyl-8-(1H-tetrazol-5-yl)-8-azabicyclo[3.2.1]octan-2-yl)urea (Compound 42″*), and Compounds 42A*, 42B*, 42C*, and 42D*

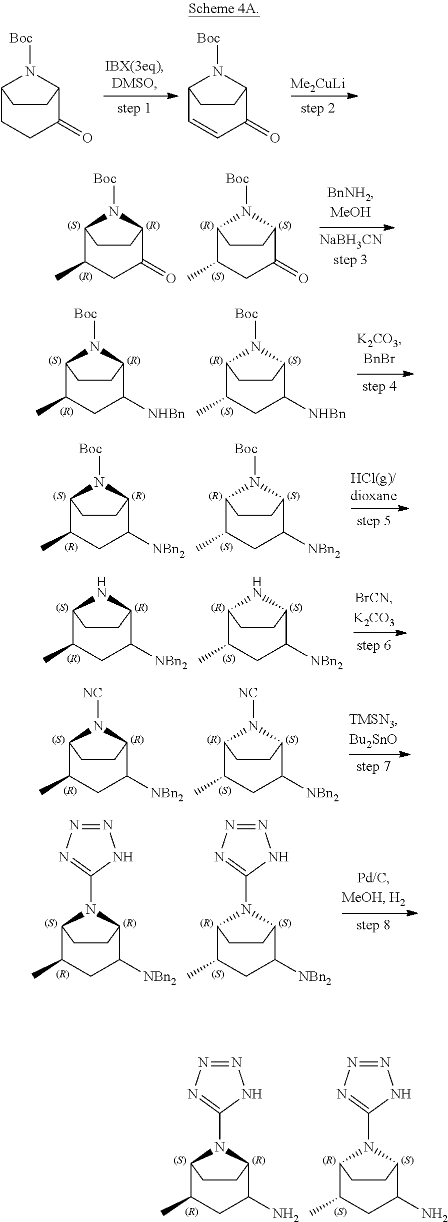

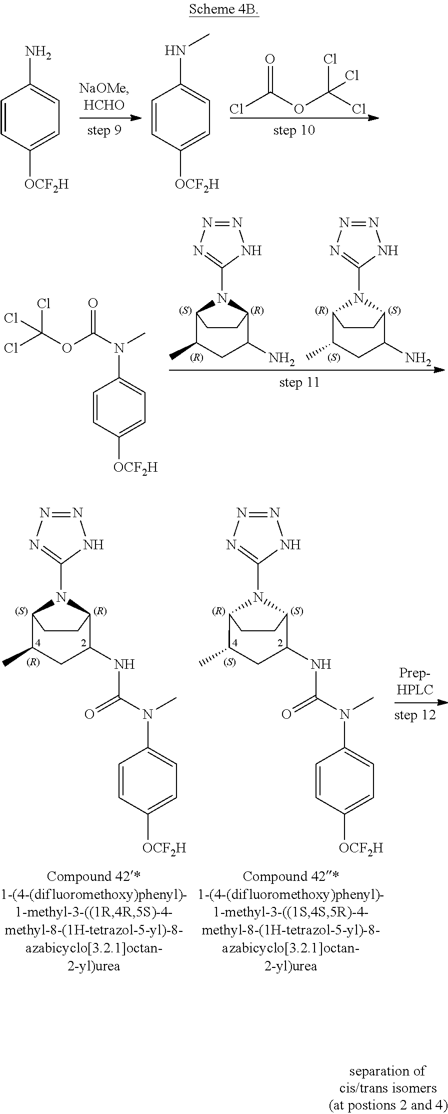

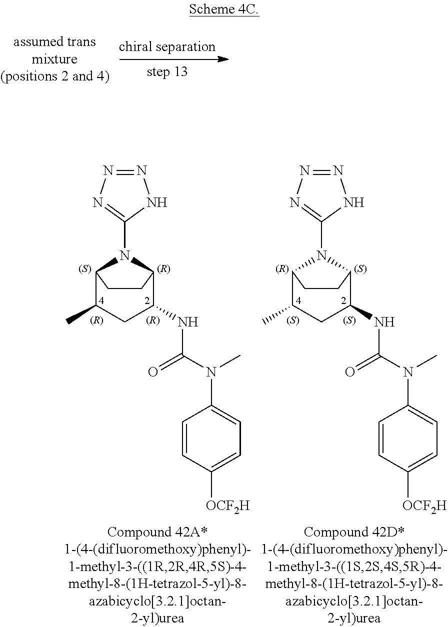

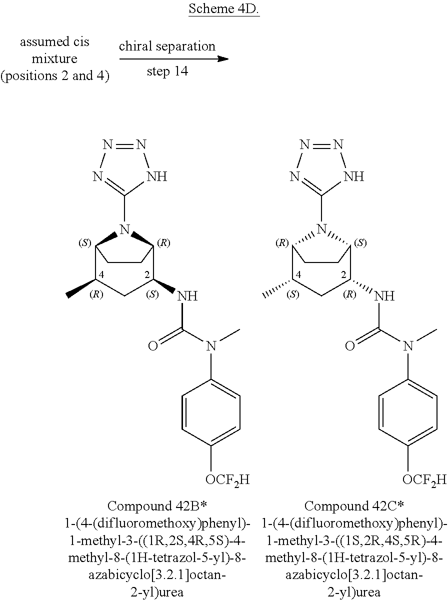

Example 4 follows Protocol B.

Step 1: Into a 20 mL vial was added tert-butyl 2-oxo-8-azabicyclo[3.2.1]octane-8-carboxylate (600 mg, 2.66 mmol, 1 equiv) and 2-iodoxybenzoic acid (IBX) (2240 mg, 7.98 mmol, 3 equiv) in dimethyl sulfoxide (DMSO) (7 mL) at room temperature. The resulting mixture was stirred for 16 h at 80° C. The reaction was monitored by LCMS until completion. The reaction was repeated twenty-four times, and the batches combined and the volatiles were removed under reduced pressure. The residue was then purified by reversed-phase flash chromatography (C18 silica gel; mobile phase, acetonitrile (MeCN) in water, 30% to 40% gradient in 10 min; detector, UV 254 nm) to provide tert-butyl 4-oxo-8-azabicyclo[3.2.1]oct-2-ene-8-carboxylate (6 g, 41% yield). LCMS: (ES, m/z) RT=0.77 min, m/z=168.1 [M+H]⁺.

Step 2: To a stirred solution of tert-butyl 4-oxo-8-azabicyclo[3.2.1]oct-2-ene-8-carboxylate (1000 mg, 4.47 mmol, 1 equiv) in tetrahydrofuran (15 mL) was added lithium dimethyl cuprate (Me₂CuLi) (0.5 M in Et₂O, 18 mL, 9 mmol, 2 equiv) dropwise at −78° C. under nitrogen atmosphere. The resulting mixture was stirred for 1 h at room temperature. The reaction was quenched with water (10 mL) at room temperature. The resulting mixture was extracted with ethyl acetate (EtOAc) (2×50 mL). The combined organic layers were washed with brine (1×20 mL), dried over anhydrous Na₂SO₄. After filtration, the filtrate was concentrated under reduced pressure. The reaction was repeated five times, and the batches combined for purification by reversed-phase flash chromatography (C18 silica gel; mobile phase, acetonitrile (MeCN) in water, 45% to 55% gradient in 10 min; detector, UV 254 nm) to provide an isomeric mixture (cis 4-Me/N-bridgehead, assumed) of tert-butyl 2-methyl-4-oxo-8-azabicyclo[3.2.1]octane-8-carboxylate (4.0 g, 63% yield). LCMS: (ES, m/z) RT=0.87 min, m/z=184.2 [M+H]⁺.

Step 3: A solution of an isomeric mixture of tert-butyl 2-methyl-4-oxo-8-azabicyclo[3.2.1]octane-8-carboxylate (trans Me-bridgehead, assumed) (1 g, 4.18 mmol, 1 equiv) and benzylamine (900 mg, 8.37 mmol, 2 equiv) in methanol (7 mL) was stirred for 16 h at 60° C. To the above mixture was added NaBH₃CN (790 mg, 12.5 mmol, 3 equiv) at 0° C. The resulting mixture was stirred for additional 1 h at 60° C. The reaction was monitored by LCMS. The reaction was quenched with water (3 mL) at room temperature. The resulting mixture was extracted with EtOAc (2×20 mL). The combined organic layers were washed with brine (1×20 mL), dried over anhydrous Na₂SO₄. After filtration, the filtrate was concentrated under reduced pressure. The reaction was repeated for three times, and the batches combined for purification by silica gel column chromatography, eluting with dichloromethane/petroleum ether (1:1) to provide an isomeric mixture of tert-butyl 2-(benzylamino)-4-methyl-8-azabicyclo[3.2.1]octane-8-carboxylate (cis 4-Me/N-bridgehead, assumed) (3 g, 55% yield). LCMS: (ES, m/z) RT=0.71 min, m/z=331.2 [M+H]⁺.

Step 4: To a stirred solution of an isomeric mixture of tert-butyl (2-(benzylamino)-4-methyl-8-azabicyclo[3.2.1]octane-8-carboxylate (cis 4-Me/N-bridgehead, assumed) (3 g, 9.07 mmol, 1 equiv) and K₂CO₃ (3160 mg, 22.7 mmol, 2.5 equiv) in acetonitrile (30 mL) was added benzyl bromide (2330 mg, 13.61 mmol, 1.50 equiv) at room temperature. The resulting mixture was stirred for 5 h at 80° C. The reaction was monitored by LCMS. The reaction was quenched with water (10 mL) at room temperature. The resulting mixture was extracted with ethyl acetate (EtOAc) (2×50 mL). The combined organic layers were washed with brine (1×30 mL), dried over anhydrous Na₂SO₄. After filtration, the filtrate was concentrated under reduced pressure to provide a residue, which was purified by silica gel column chromatography, eluting with dichloromethane/petroleum ether (1:2) to provide an isomeric mixture of tert-butyl (2-(dibenzylamino)-4-methyl-8-azabicyclo[3.2.1]octane-8-carboxylate (cis 4-Me/N-bridgehead, assumed) (3.5 g, 92% yield). LCMS: (ES, m/z) RT=0.80 min, m/z=421.2 [M+H]⁺.

Step 5: Into a 100 mL round-bottom flask was added an isomeric mixture of tert-butyl (2-(dibenzylamino)-4-methyl-8-azabicyclo[3.2.1]octane-8-carboxylate (cis 4-Me/N-bridgehead, assumed) (3.5 g, 8.32 mmol, 1 equiv) and HCl(gas)(4M) in 1,4-dioxane (30 mL) at room temperature. The resulting mixture was stirred for 1 h at room temperature. The reaction was monitored by LCMS. The resulting mixture was concentrated under reduced pressure to provide crude isomeric mixture of N,N-dibenzyl-4-methyl-8-azabicyclo[3.2.1]octan-2-amine (cis 4-Me/N-bridgehead, assumed), which was used in next step directly without purification. LCMS: (ES, m/z) RT=0.52 min, m/z=321.0[M+H]⁺.

Step 6: Into a 100 mL round-bottom flask was added N,N-dibenzyl-4-methyl-8-azabicyclo[3.2.1]octan-2-amine (3 g, 9.36 mmol, 1 equiv), cyanogen bromide (1.98 g, 18.7 mmol, 2 equiv), K₂CO₃ (3.91 g, 28.1 mmol, 3 equiv), and acetonitrile (30 mL) at room temperature. The resulting mixture was stirred for 2 h at 80° C. The reaction was monitored by LCMS. The reaction was quenched with water (10 mL) at room temperature. The resulting mixture was extracted with ethyl acetate (EtOAc) (3×30 mL). The combined organic layers were washed with brine (1×30 mL), dried over anhydrous Na₂SO₄. After filtration, the filtrate was concentrated under reduced pressure to provide a crude isomeric mixture of 2-(dibenzylamino)-4-methyl-8-azabicyclo[3.2.1]octane-8-carbonitrile (cis 4-Me/N-bridgehead, assumed), which was used in the next step directly without purification. LCMS: (ES, m/z) RT=0.78 min, m/z=346.0[M+H]⁺.

Step 7: Into a 40 mL vial was added an isomeric mixture of 2-(dibenzylamino)-4-methyl-8-azabicyclo[3.2.1]octane-8-carbonitrile (cis 4-Me/N-bridgehead, assumed) (2.30 g, 6.65 mmol, 1 equiv), dibutyltin oxide (1.66 g, 6.65 mmol, 1 equiv), trimethylsilyl azide (2.30 g, 20.0 mmol, 3 equiv), and dimethylformamide (DMF) (20 mL) at room temperature. The resulting mixture was stirred for 2 h at 120° C. under nitrogen atmosphere. The reaction was monitored by LCMS. The residue was purified by reversed-phase flash chromatography (C18 silica gel; mobile phase, acetonitrile in water (0.1% trifluoroacetic acid), 30% to 50% gradient in 10 min; detector, UV 254 nm) to provide an isomeric mixture of N,N-dibenzyl-4-methyl-8-(1H-1,2,3,4-tetrazol-5-yl)-8-azabicyclo[3.2.1]octan-2-amine (cis 4-Me/N-bridgehead, assumed) (2.10 g, 72% yield). LCMS: (ES, m/z) RT=0.74 min, m/z=389.0[M+H]⁺.

Step 8: Into a 100 mL round-bottom flask was added an isomeric mixture of N,N-dibenzyl-4-methyl-8-(1H-1,2,3,4-tetrazol-5-yl)-8-azabicyclo[3.2.1]octan-2-amine (cis 4-Me/N-bridgehead, assumed) (2 g, 5.14 mmol, 1 equiv) in methanol (MeOH) (20 mL) was added Pd/C (1.36 g, 10.28 mmol, 2 equiv), and HCl (12M) (0.5 mL) at room temperature. The resulting mixture was stirred for 6 h at room temperature under hydrogen atmosphere. The reaction was monitored by LCMS. The resulting mixture was filtered, the filter cake was washed with MeOH (2×25 mL). The filtrate was concentrated under reduced pressure to provide an isomeric mixture of 4-methyl-8-(1H-1,2,3,4-tetrazol-5-yl)-8-azabicyclo[3.2.1]octan-2-amine, HCl salt (cis 4-Me/N-bridgehead, assumed) (1.20 g, 96% yield). LCMS: (ES, m/z) RT=0.25 min, m/z=209.0[M+H]⁺.

Step 9: Into a 250 mL round-bottom flask were added 4-(difluoromethoxy)aniline (1 g, 6.28 mmol, 1 equiv) and sodium methoxide (NaOMe) (1.02 g, 18.9 mmol, 3 equiv), formaldehyde (HCHO) (40% in water, 1.26 g, 12.6 mmol, 2 equiv), and methanol (MeOH) (20 mL) at room temperature. The resulting mixture was stirred for 16 h at room temperature, followed by the addition of NaBH₄ (0.36 g, 9.43 mmol, 1.5 equiv) in portions at 0° C. The resulting mixture was stirred for 1 h at room temperature. The reaction was monitored by LCMS. The reaction was quenched by the addition of water (20 mL) at 0° C. The aqueous layer was extracted with ethyl acetate (EtOAc) (3×10 mL). The resulting mixture was concentrated under reduced pressure to provide a residue, which was purified by silica gel column chromatography, eluting with petroleum ether/ethyl acetate (6:1) to provide 4-(difluoromethoxy)-N-methylaniline (“aniline (iv) reagent”) (1 g, 92% yield). LCMS: (ES, m/z) RT=0.422 min, m/z=174[M+H]⁺.

Step 10: Into a 250 mL round-bottom flask was added 4-(difluoromethoxy)-N-methylaniline (1 g, 5.77 mmol, 1 equiv), triethylamine (1.75 g, 17.32 mmol, 3 equiv), and dichloromethane (DCM) (20 mL) at room temperature, followed by the addition of ClC(═O)OCCl₃ (diphosgene) (1.71 g, 8.66 mmol, 1.5 equiv) dropwise at 0° C. The resulting mixture was stirred for 2 h at room temperature. The reaction was monitored by LCMS. The resulting mixture was concentrated under reduced pressure to provide a residue, which was purified by silica gel column chromatography, eluting with petroleum ether/ethyl acetate (10:1) to provide trichloromethyl N-[4-(difluoromethoxy)phenyl]-N-methylcarbamate (1 g, 52% yield).

Step 11: Into a 25 mL round-bottom flask were added an isomeric mixture of 4-methyl-8-(1H-1,2,3,4-tetrazol-5-yl)-8-azabicyclo[3.2.1]octan-2-amine hydrochloride (“amine (i) reagent”) (cis 4-Me/N-bridgehead, assumed) (80 mg, 0.32 mmol, 1 equiv), K₂CO₃ (135.53 mg, 0.98 mmol, 3 equiv), and dimethylformamide (DMF) (2 mL) at room temperature, then trichloromethyl N-[4-(difluoromethoxy)phenyl]-N-methylcarbamate (164 mg, 0.49 mmol, 1.5 equiv) in dimethylformamide (DMF) (1 mL) was added dropwise at 0° C. The resulting mixture was stirred for 2 h at room temperature. The reaction was monitored by LCMS. The volatiles were removed under reduced pressure and the residue was directly purified by reversed-phase flash chromatography (C18 silica gel; mobile phase, acetonitrile in water (0.1% trifluoroacetic acid), 10% to 50% gradient in 10 min; detector, UV 254 nm) to provide an isomeric mixture (cis 4-Me/N-bridgehead, assumed) of Compound 42′* and Compound 42″* (50 mg, 38% yield). LCMS: (ES, m/z) RT=0.622 min, m/z=408[M+H]⁺. Stereochemistry was arbitrarily assigned.

Step 12: The crude product, a mixture of Compound 42′* and Compound 42″*, was purified by Prep HPLC (Column: XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: 15% B to 25% B in 10 min; Wave Length: 254 nm/220 nm nm) to provide a first eluting isomeric mixture (cis at the 2 and 4 position, assumed, RT(min): 9.5, 35 mg, 43% yield) and a second eluting isomeric mixture (trans at the 2 and 4 position, assumed, RT(min): 13.5, 15 mg, 19% yield). LCMS: (ES, m/z) RT=0.624 min, m/z=408[M+H]⁺.

Step 13: The second eluting isomeric mixture (trans at the 2 and 4 position, assumed) (15 mg) was purified by chiral-Prep HPLC (Column: CHIRAL ART Cellulose-SC, 2*25 cm, 5 m; Mobile Phase A: hexanes (0.1% trifluoroacetic acid), Mobile Phase B: ethanol (EtOH): dichloromethane (DCM)=1:1; Flow rate: 20 mL/min; Gradient: 30% B to 30% B in 10 min; Wave Length: 220/254 nm) to provide Compound 42A* (RT(min)=7.54, 3.9 mg) and Compound 42D* (RT(min)=9.26, 5 mg); Stereochemistry arbitrarily assigned.

Compound 42A*: LCMS: (ES, m/z) RT=0.813 min, m/z=408[M+H]⁺. ¹H NMR (400 MHz, Methanol-d4) δ 7.38-7.30 (m, 2H), 7.24 (d, J=8.8 Hz, 2H), 6.87 (t, J=73.9 Hz, 1H), 4.24 (d, J=5.4 Hz, 1H), 4.13-4.03 (m, 2H), 3.26 (s, 3H), 2.00-1.86 (m, 2H), 1.83-1.74 (m, 1H), 1.74-1.62 (m, 3H), 1.45 (dd, J=13.6, 5.4 Hz, 1H), 1.12 (d, J=7.0 Hz, 3H).

Compound 42D*: LCMS: (ES, m/z) RT=0.808 min, m/z=408[M+H]⁺. ¹H NMR (400 MHz, Methanol-d4) δ 7.38-7.30 (m, 2H), 7.27-7.20 (m, 2H), 6.87 (t, J=73.9 Hz, 1H), 4.24 (s, 1H), 4.09 (t, J=8.9 Hz, 2H), 3.26 (d, J=1.0 Hz, 3H), 2.01-1.86 (m, 2H), 1.85-1.76 (m, 1H), 1.74-1.62 (m, 3H), 1.45 (d, J=13.6 Hz, 1H), 1.12 (d, J=7.0 Hz, 3H).

Step 14: The first eluting isomeric mixture (cis at the 2 and 4 position, assumed) (35 mg) was purified by chiral-Prep HPLC (Column: CHIRALPAK ID, 2*25 cm, 5 m; Mobile Phase A: hexanes (0.1% trifluoroacetic acid), Mobile Phase B: ethanol (EtOH): dichloromethane (DCM)=1:1; Flow rate: 20 mL/min; Gradient: 30% B to 30% B in 7.5 min; Wave Length: 220/254 nm) to provide Compound 42B* (RT(min)=4.73, 9.3 mg) and Compound 42C* (RT(min)=5.96, 11.6 mg); Stereochemistry arbitrarily assigned.

Compound 42B*: LCMS: (ES, m/z) RT=0.733 min, m/z=408[M+H]⁺. 1H NMR (400 MHz, Methanol-d4) δ 7.28-7.19 (m, 2H), 7.12-7.03 (m, 2H), 6.79 (t, J=73.7 Hz, 1H), 4.26 (d, J=4.5 Hz, 1H), 3.96 (s, 1H), 3.79 (t, J=4.4 Hz, 1H), 3.23 (s, 3H), 2.13 (t, J=6.2 Hz, 1H), 1.97 (d, J=9.8 Hz, 2H), 1.82 (d, J=12.3 Hz, 3H), 1.38-1.32 (m, 1H), 0.81 (d, J=7.2 Hz, 3H).

Compound 42C*: LCMS: (ES, m/z) RT=0.730 min, m/z=408[M+H]⁺. 1H NMR (400 MHz, Methanol-d4) δ 7.28-7.19 (m, 2H), 7.12-7.03 (m, 2H), 6.79 (t, J=73.7 Hz, 1H), 4.26 (s, 1H), 3.96 (s, 1H), 3.79 (s, 1H), 3.23 (d, J=0.9 Hz, 3H), 2.13 (d, J=15.1 Hz, 1H), 2.00-1.93 (m, 2H), 1.87-1.74 (m, 3H), 1.32 (d, J=15.0 Hz, 1H), 0.81 (d, J=7.3 Hz, 3H).

### Example 5. 1-(4-(difluoromethoxy)-2-fluorophenyl)-1-ethyl-3-((1R,2R,4R,5S)-4-methyl-8-(1H-tetrazol-5-yl)-8-azabicyclo[3.2.1]octan-2-yl)urea (Compound 65A*) and 1-(4-(difluoromethoxy)-2-fluorophenyl)-1-ethyl-3-((1S,2S,4S,5R)-4-methyl-8-(1H-tetrazol-5-yl)-8-azabicyclo[3.2.1]octan-2-yl)urea (Compound 65D*)

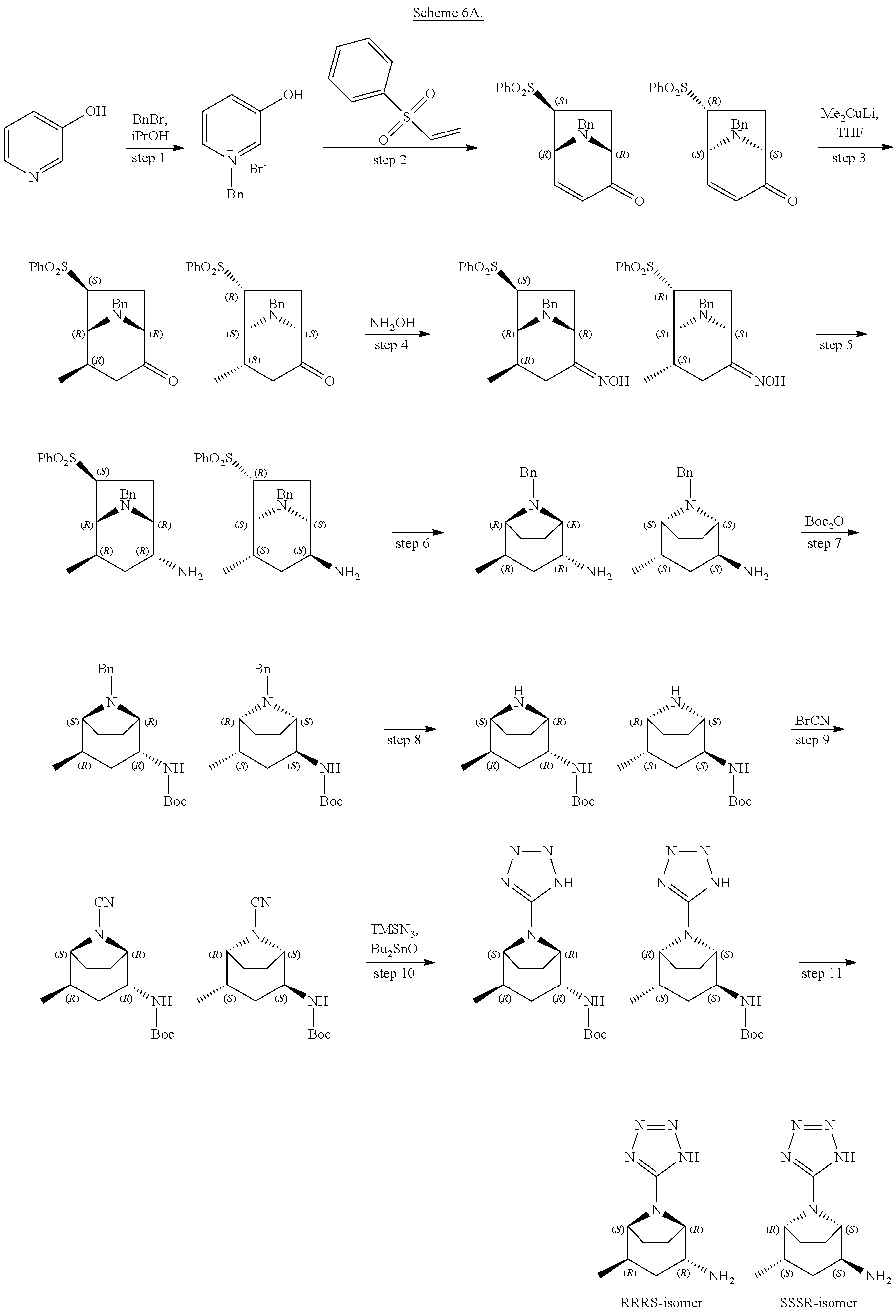

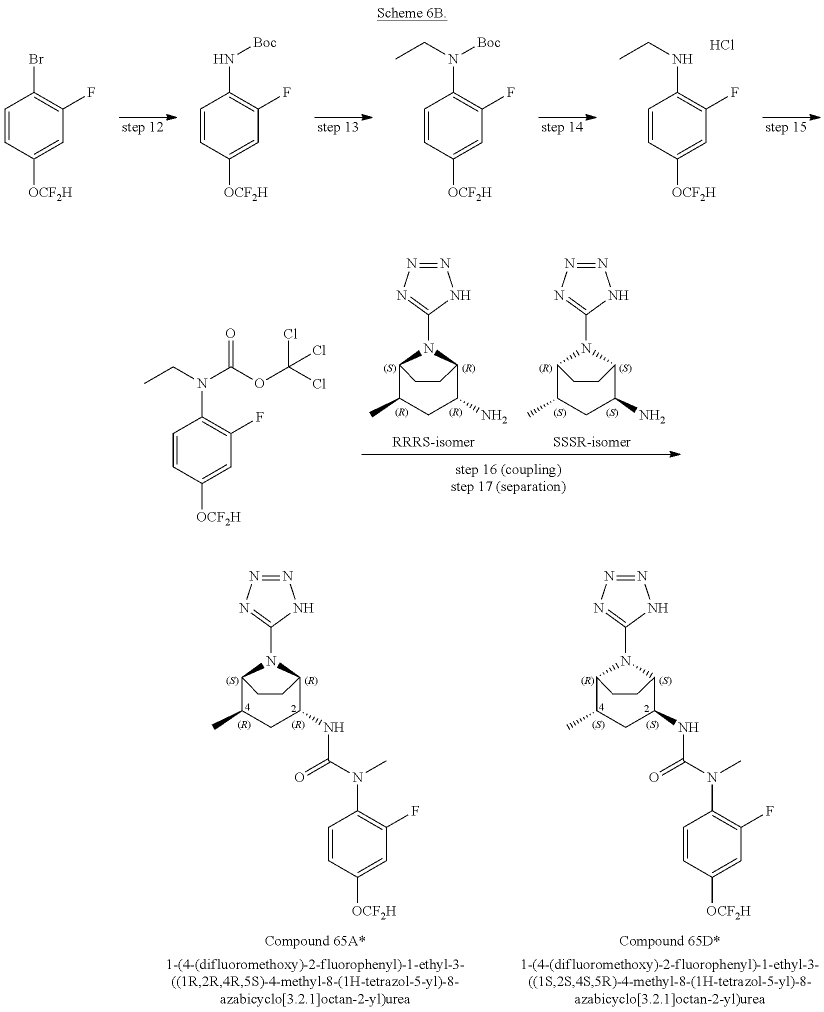

Example 5 follows Protocol B.

Step 1: Into a 1 L 3-necked round-bottom flask were added 3-hydroxypyridine (50.0 g, 526 mmol, 1 equiv), isopropanol (IPA) (500 mL), and benzyl bromide (BnBr) (96.2 g, 562 mmol, 1.07 equiv) at room temperature. The resulting mixture was stirred overnight at 80° C., then the resulting mixture was concentrated under reduced pressure. The residue was purified by washing with ethyl acetate (EtOAc) (200 mL). The solid was collected and dried over under vacuum. This resulted in 1-benzyl-3-hydroxypyridin-1-ium bromide (135 g, 97% yield). LCMS: (ES, m/z): RT=0.47 min, m/z=186.1[M+H]⁺.

Step 2: To a stirred solution of 1-benzyl-3-hydroxypyridin-1-ium bromide (25.0 g, 93.9 mmol, 1 equiv), (ethenesulfonyl)benzene (21.0 g, 125 mmol, 1.33 equiv), hydroquinone (207 mg, 1.88 mmol, 0.02 equiv) in tetrahydrofuran (THF) (200 mL) was added triethylamine (NEt₃) (14.2 g, 141 mmol, 1.50 equiv) dropwise at room temperature under nitrogen atmosphere. The resulting mixture was stirred overnight at 65 C under nitrogen atmosphere. The reaction was quenched by the addition of water (300 mL) at room temperature. The aqueous layer was extracted with ethyl acetate (EtOAc) (3×200 mL). The organic phase was concentrated under reduced pressure and the solid was precipitated. The precipitated solid was collected and washed by EtOAc (3×50 mL). This resulted in a mixture of assumed (1R,5R,6S)-8-benzyl-6-(phenylsulfonyl)-8-azabicyclo[3.2.1]oct-3-en-2-one and the corresponding enantiomer (20 g, 60% yield). LCMS: (ES, m/z): RT=0.91 min, m/z=354.1[M+H]⁺.

Step 3: To a stirred solution of the mixture of step 2 (20 g, 56.6 mmol, 1 equiv) in 200 mL tetrahydrofuran (THF) was added dimethyl copper lithium (Me₂CuLi) (0.5M in diethylether (Et₂O)) (169.8 mL, 84.88 mmol, 1.50 equiv) dropwise at −78° C. under nitrogen atmosphere. The resulting mixture was stirred for 1 h at room temperature under nitrogen atmosphere. The reaction was quenched by the addition of water (500 mL) at 0° C. The resulting mixture was extracted with ethyl acetate (EtOAc) (3×500 mL). The combined organic layers were washed with water (2×300 mL), dried over anhydrous Na₂SO₄, filtered, and the filtrate was concentrated under reduced pressure to provide a mixture of assumed (1R,4R,5R,6S)-8-benzyl-4-methyl-6-(phenylsulfonyl)-8-azabicyclo[3.2.1]octan-2-one and the corresponding enantiomer (18.0 g, 86% yield). LCMS: (ES, m/z): RT=1.01 min, m/z=370.1[M+H]⁺.

Step 4: Into a 100 mL 3-necked round-bottom flask was added the mixture of step 3 (2.58 g, 6.98 mmol, 1 equiv) and hydroxylamine hydrochloride (534 mg, 7.68 mmol, 1.1 equiv), pyridine (1104 mg, 13.96 mmol, 2 equiv), and ethanol (EtOH) (25.8 mL) at room temperature. The resulting mixture was stirred overnight at room temperature. The reaction was quenched by the addition of water (100 mL) at room temperature. The resulting mixture was extracted with ethyl acetate (EtOAc) (3×100 mL). The combined organic layers were washed with water (2×50 mL), dried over anhydrous Na₂SO₄. After filtration, the filtrate was concentrated under reduced pressure. The solid was precipitated and precipitated solid was collected to provide a mixture of assumed N-[(1R,4R,5R,6S)-6-(benzenesulfonyl)-8-benzyl-4-methyl-8-azabicyclo[3.2.1]octan-2-ylidene]hydroxylamine and the corresponding enantiomer (2.5 g, 93% yield). LCMS: (ES, m/z): RT=0.78 min, m/z=385.1[M+H]⁺.

Step 5: A solution of the mixture of step 4 (2.50 g, 6.50 mmol, 1 equiv) in dimethylformamide (DMF) was treated with NaBH₃CN (1.63 g, 26.0 mmol, 4 equiv), sodium bisulfate (2.34 g, 19.5 mmol, 3.00 equiv) for 5 min at room temperature under nitrogen atmosphere followed by the addition of molybdenum pentachloride (0.89 g, 3.25 mmol, 0.5 equiv) in portions at room temperature. The resulting mixture was stirred overnight at room temperature under nitrogen atmosphere. The reaction was quenched by the addition of sat. NaHCO₃ (aq.) (100 mL) at room temperature. The resulting mixture was extracted with ethyl acetate (EtOAc) (3×100 mL). The combined organic layers were washed with water (2×50 mL) and dried over anhydrous Na₂SO₄. After filtration, the filtrate was concentrated under reduced pressure to provide a mixture of assumed (1R,2R,4R,5R,6S)-8-benzyl-4-methyl-6-(phenylsulfonyl)-8-azabicyclo[3.2.1]octan-2-amine and the corresponding enantiomer (1.80 g, 75% yield). LCMS: (ES, m/z): RT=0.65 min, m/z=371.1[M+H]⁺.

Step 6: A solution of the mixture of step 5 (1 g, 2.69 mmol, 1 equiv) and magnesium chips (treated with 0.5% HCl) (0.52 g, 22 mmol, 7.9 equiv) in methanol (MeOH) (15 mL) was stirred overnight at room temperature. To the above mixture was added acetic acid (AcOH) (0.66 mL) and H₂O (10 mL) at room temperature. The resulting mixture was stirred for an additional 1 h at room temperature. The reaction was quenched by the addition of sat. NaOH (aq.) (5 mL) at room temperature. The resulting mixture was extracted with ethyl acetate (EtOAc) (3×10 mL), and the combined organic layers were washed with water (3×10 mL), dried over anhydrous Na₂SO₄, filtered, and the filtrate was concentrated under reduced pressure to provide a mixture of assumed (1R,2R,4R,5S)-8-benzyl-4-methyl-8-azabicyclo[3.2.1]octan-2-amine and the corresponding enantiomer (700 mg, 84% yield). LCMS: (ES, m/z): RT=0.34 min, m/z=231.1[M+H]⁺.

Step 7: Into an 8 mL vial was added the mixture of step 6 (700 mg, 3.03 mmol, 1.00 equiv), methanol (MeOH) (3 mL), and ditertbutyldicarbonate (Boc₂O) (796 mg, 3.64 mmol, 1.20 equiv) at room temperature. The resulting mixture was stirred at room temperature for 2 h. The resulting mixture was concentrated under reduced pressure. The residue was purified by silica gel column chromatography, eluting with petroleum ether/ethyl acetate (5:1), to afford a mixture of assumed tert-butyl ((1R,2R,4R,5S)-8-benzyl-4-methyl-8-azabicyclo[3.2.1]octan-2-yl)carbamate and the corresponding enantiomer (800 mg, 80% yield). LCMS: (ES, m/z): RT=0.62 min, m/z=331.1[M+H]⁺.

Step 8: To a stirred solution of the mixture of step 7 (4.30 g, 13.0 mmol, 1 equiv) in isopropanol (i-PrOH) (50 mL) was added Pd/C (wet, 10% on carbon, 2.76 g) at room temperature. The resulting mixture was stirred for 16 h at room temperature under hydrogen atmosphere. The resulting mixture was filtered, the filter cake was washed with methanol (MeOH) (3×50 mL). The filtrate was concentrated under reduced pressure to afford a mixture of assumed tert-butyl ((1R,2R,4R,5S)-4-methyl-8-azabicyclo[3.2.1]octan-2-yl)carbamate and the corresponding enantiomer (2.7 g, 86% yield). LCMS: (ES, m/z): RT=0.50 min, m/z=241.2[M+H]⁺.

Step 9: To a stirred solution of the mixture of step 8 (2.70 g, 11.2 mmol, 1 equiv) and K₂CO₃ (4.69 g, 33.7 mmol, 3.00 equiv) in acetonitrile (30 mL) was added cyanogen bromide (4.76 g, 44.9 mmol, 4.00 equiv) at room temperature. The resulting mixture was stirred for 2 h at 80° C. The reaction was quenched with water (50 mL) at room temperature. The resulting mixture was extracted with ethyl acetate (EtOAc) (2×100 mL). The combined organic layers were washed with brine (1×50 mL), dried over anhydrous Na₂SO₄, filtered, and the filtrate was concentrated under reduced pressure to afford a mixture of assumed tert-butyl ((1R,2R,4R,5S)-8-cyano-4-methyl-8-azabicyclo[3.2.1]octan-2-yl)carbamate and the corresponding enantiomer (2.7 g, 91% yield). LCMS: (ES, m/z): RT=0.66 min, m/z=266.2[M+H]⁺.

Step 10: To a stirred solution of the mixture of step 9 (1.30 g, 4.89 mmol, 1 equiv) and dibutyltin oxide (Bu₂SnO) (2.43 g, 9.79 mmol, 2 equiv) in dimethylformamide (DMF) (13 mL) was added trimethylsilyl azide (1.12 g, 9.79 mmol, 2 equiv) at room temperature. The resulting mixture was stirred for 2 h at 120° C. The residue was purified by reversed-phase flash chromatography (C18 silica gel; mobile phase, MeCN in water (0.1% TFA), 30% to 50% gradient in 10 min; detector, UV 254/220 nm) to afford a mixture of assumed tert-butyl ((1R,2R,4R,5S)-4-methyl-8-(1H-tetrazol-5-yl)-8-azabicyclo[3.2.1]octan-2-yl)carbamate and the corresponding enantiomer (0.90 g, 60% yield). LCMS: (ES, m/z): RT=0.67 min, m/z=309.1[M+H]⁺.

Step 11: Into a 250 mL round-bottom flask was added the mixture of step 10 (0.90 g, 2.9 mmol, 1.0 equiv) and HCl (gas) in 1,4-dioxane (4M, 10.0 mL) at room temperature. The resulting mixture was stirred for 2 h at room temperature. The resulting mixture was concentrated under reduced pressure to afford a mixture of assumed (1R,2R,4R,5S)-4-methyl-8-(1H-tetrazol-5-yl)-8-azabicyclo[3.2.1]octan-2-amine hydrochloride salt (RRRS-isomer) and (1S,2S,4S,5R)-4-methyl-8-(1H-tetrazol-5-yl)-8-azabicyclo[3.2.1]octan-2-amine hydrochloride salt (SSSR-isomer) (0.80 g, 95% yield), collectively referred to as the “amine (i) reagent”. LCMS: (ES, m/z): RT=0.15 min, m/z=209.1[M+H]⁺.

Step 12: A mixture of 1-bromo-4-(difluoromethoxy)-2-fluorobenzene (1 g, 4.15 mmol, 1 equiv), tert-butyl carbamate (972 mg, 8.30 mmol, 2 equiv), dicyclohexyl[2′,4′,6′-tris(propan-2-yl)[1,1′-biphenyl]-2-yl]phosphane (XPhos) (396 mg, 0.830 mmol, 0.2 equiv), (2-Dicyclohexylphosphino-2′,4′,6′-triisopropyl-1,1′-biphenyl)[2-(2′-amino-1,1′-biphenyl)]palladium(II) methanesulfonate (XPhos Pd G3) (350 mg, 0.42 mmol, 0.10 equiv) and Cs₂CO₃ (4.06 g, 12.5 mmol, 3.00 equiv) in dioxane (10 mL) was stirred for 2 h at 100° C. under nitrogen atmosphere. The reaction was quenched by the addition of water (50 mL) at room temperature. The resulting mixture was extracted with ethyl acetate (EtOAc) (3×200 mL). The combined organic layers were washed with brine (2×200 mL), dried over anhydrous Na₂SO₄. After filtration, the filtrate was concentrated under reduced pressure. The residue was purified by silica gel column chromatography, eluting with petroleum ether/ethyl acetate (5:1) to afford tert-butyl N-[4-(difluoromethoxy)-2-fluorophenyl]carbamate (930 mg, 77% yield). LCMS: (ES, m/z): RT=0.95 min, m/z=276.0[M−H]—.

Step 13: Into a 40 mL vial were added tert-butyl N-[4-(difluoromethoxy)-2-fluorophenyl]carbamate (800 mg, 2.89 mmol, 1 equiv) and tetrahydrofuran (THF) (9 mL) at room temperature. To the above mixture was added NaH (60% dispersion in mineral oil, 139 mg, 5.77 mmol, 2 equiv) dropwise at 0° C. The resulting mixture was stirred for an additional 30 min at 0° C. To the above mixture was added ethyl iodide (675 mg, 4.33 mmol, 1.5 equiv) at 0° C. The resulting mixture was stirred for additional 1 h at room temperature. The reaction was quenched with sat. NH₄Cl (aq.) (50 mL) at room temperature. The resulting mixture was extracted with ethyl acetate (EtOAc) (3×250 mL). The combined organic layers were washed with water (3×50 mL) and dried over anhydrous Na₂SO₄. After filtration, the filtrate was concentrated under reduced pressure. The residue was purified by silica gel column chromatography, eluting with petroleum ether/ethyl acetate (1:1), to afford tert-butyl N-[4-(difluoromethoxy)-2-fluorophenyl]-N-ethylcarbamate (700 mg, 76% yield). LCMS: (ES, m/z): RT=1.02 min, m/z=306.1[M+H]⁺.

Step 14: Into an 20 mL vial were added tert-butyl N-[4-(difluoromethoxy)-2-fluorophenyl]-N-ethylcarbamate (600 mg, 1.97 mmol, 1 equiv) and HCl(gas) in 1,4-dioxane (4M, 7 mL) for 1 h at room temperature. The resulting mixture was concentrated under reduced pressure to provide 4-(difluoromethoxy)-N-ethylaniline hydrochloride (“aniline (iv) reagent”) (350 mg, 92% yield). LCMS: (ES, m/z): RT=0.89 min, m/z=206.1[M+H]⁺.

Step 15: Into an 20 mL vial were added 4-(difluoromethoxy)-N-ethylaniline hydrochloride (300 mg, 1.60 mmol, 1 equiv), triethylamine (TEA) (324 mg, 3.21 mmol, 2 equiv) and dichloromethane (DCM) (3 mL) at room temperature. To the above mixture was added trichloromethyl carbonochloridate (476 mg, 2.41 mmol, 1.5 equiv) by dropwise at 0° C. The resulting mixture was stirred for additional 1 h at room temperature. The reaction was quenched by the addition of water (30 mL) at room temperature. The resulting mixture was extracted with ethyl acetate (EtOAc) (3×100 mL). The combined organic layers were washed with brine (3×50 mL) and dried over anhydrous Na₂SO₄. After filtration, the filtrate was concentrated under reduced pressure. The residue was purified by silica gel column chromatography, eluting with petroleum ether/ethyl acetate (1:1), to afford trichloromethyl N-[4-(difluoromethoxy)-2-fluorophenyl]-N-ethylcarbamate (300 mg, 50% yield). LCMS: (ES, m/z): RT=0.99 min, m/z=366.1[M+H]⁺.

Step 16: Into an 20 mL vial were added trichloromethyl N-[4-(difluoromethoxy)-2-fluorophenyl]-N-ethylcarbamate (200 mg, 0.55 mmol, 1 equiv), the “amine (i) reagent” of step 11 (200 mg, 0.82 mmol, 1.50 equiv), diisopropylethyl amine (DIEA) (353 mg, 2.73 mmol, 5 equiv) and acetonitrile (4 mL) for 1 h at room temperature. The reaction was monitored by LCMS. The reaction was quenched by the addition of water (30 mL) at room temperature. The resulting mixture was extracted with ethyl acetate (EtOAc) (2×100 mL). The combined organic layers were washed with brine (2×50 mL) and dried over anhydrous Na₂SO₄. After filtration, the filtrate was concentrated under reduced pressure to provide a residue (150 mg, 90% purity) which was purified by Prep-HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 25% B to 35% B in 10 min; Wave Length: 254/220 nm; RT(min): 7.28) to afford a mixture of 1-(4-(difluoromethoxy)-2-fluorophenyl)-1-ethyl-3-((1R,2R,4R,5S)-4-methyl-8-(1H-tetrazol-5-yl)-8-azabicyclo[3.2.1]octan-2-yl)urea (Compound 65A*) and 1-(4-(difluoromethoxy)-2-fluorophenyl)-1-ethyl-3-((1S,2S,4S,5R)-4-methyl-8-(1H-tetrazol-5-yl)-8-azabicyclo[3.2.1]octan-2-yl)urea (Compound 65D*) (100 mg). LCMS: (ES, m/z): RT=0.71 min, m/z=440.1[M+H]⁺.

Step 17: The mixture of step 16 (100 mg, 97% purity) was purified by Chiral HPLC (CHIRALPAK IC, 2*25 cm, 5 m; Mobile Phase A: Hexanes (0.1% TFA), Mobile Phase B: ethanol:dichloromethane=1:1; Flow rate: 20 mL/min; Gradient: 25% B to 25% B in 15 min; Wave Length: 220/254 nm) to afford Compound 65A* (31.7 mg, RT(min): 9.32) as the first eluting peak, and Compound 65D* (33.1 mg, RT(min): 12.57) as the second eluting peak. Stereochemistry arbitrarily assigned.

Compound 65A*: LCMS: (ES, m/z): RT=0.83 min, m/z=440.2[M+H]⁺. ¹H NMR (400 MHz, DMSO-d₆) δ 7.53-7.12 (m, 3H), 7.10-7.02 (m, 1H), 5.82 (d, J=7.6 Hz, 1H), 4.27-4.16 (m, 1H), 4.04-3.88 (m, 2H), 3.63-3.46 (m, 2H), 1.91-1.60 (m, 4H), 1.58-1.38 (m, 2H), 1.32-1.17 (m, 1H), 1.15-0.92 (m, 6H).

Compound 65D*: LCMS: (ES, m/z): RT=0.83 min, m/z=440.2[M+H]⁺. ¹H NMR (400 MHz, DMSO-d₆) δ 7.53-7.14 (m, 3H), 7.10-7.03 (m, 1H), 5.82 (d, J=7.6 Hz, 1H), 4.28-4.17 (m, 1H), 4.03-3.88 (m, 2H), 3.61-3.45 (m, 2H), 1.88-1.60 (m, 4H), 1.59-1.41 (m, 2H), 1.32-1.18 (m, 1H), 1.11-0.92 (m, 6H).

### Example 6. 1-(2-fluoro-4-(trifluoromethoxy)phenyl)-1-methyl-3-((1R,2R,4R,5S)-4-methyl-8-(1H-tetrazol-5-yl)-8-azabicyclo[3.2.1]octan-2-yl)urea (Compound 56A*) and 1-(2-fluoro-4-(trifluoromethoxy)phenyl)-1-methyl-3-((1S,2S,4S,5R)-4-methyl-8-(1H-tetrazol-5-yl)-8-azabicyclo[3.2.1]octan-2-yl)urea (Compound 56D*)

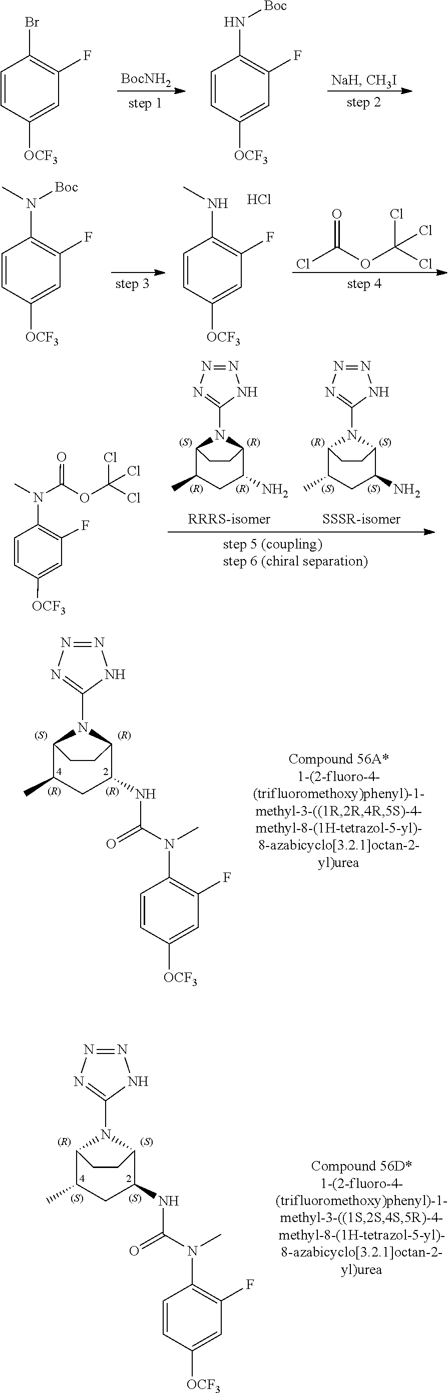

Example 6 follows Protocol B.

Step 1: Into a 500 mL round-bottom flask were added 1-bromo-2-fluoro-4-(trifluoromethoxy)benzene (10.0 g, 38.6 mmol, 1 equiv), tert-butyl carbamate (6.78 g, 57.9 mmol, 1.5 equiv), dicyclohexyl[2′,4′,6′-tris(propan-2-yl)[1,1′-biphenyl]-2-yl]phosphane (XPhos) (3.68 g, 7.72 mmol, 0.2 equiv), Cs₂CO₃ (37.74 g, 115.83 mmol, 3 equiv) and (2-dicyclohexylphosphino-2′,4′,6′-triisopropyl-1,1′-biphenyl)[2-(2′-amino-1,1′-biphenyl)]palladium(II) methanesulfonate (XPhos Pd G3) (3.27 g, 3.86 mmol, 0.1 equiv) in 1,4-dioxane (100 mL) at room temperature. The resulting mixture was stirred for 2 h at 100° C. under nitrogen atmosphere. The reaction was monitored by LCMS, quenched by the addition of water (200 mL) at room temperature, and the resulting mixture was extracted with ethyl acetate (EtOAc) (3×200 mL). The combined organic layers were washed with water (2×200 mL), dried over anhydrous Na₂SO₄, and concentrated under reduced pressure. The residue was purified by reversed-phase flash chromatography (C18 silica gel; mobile phase, CH₃CN in water, 0% to 100% gradient in 30 min; detector, UV 220/254 nm) to afford tert-butyl N-[2-fluoro-4-(trifluoromethoxy)phenyl]carbamate (8 g, 70% yield). LCMS: (ES, m/z): RT=0.965 min, m/z=296.1 [M+H]⁺.

Step 2: Into a tetrahydrofuran (THF) (80 mL) solution of tert-butyl N-[2-fluoro-4-(trifluoromethoxy)phenyl]carbamate (8.00 g, 27.07 mmol, 1 equiv) was added NaH (60% in mineral oil, 3.25 g, 81.21 mmol, 3 equiv) by batches at 0° C. The resulting mixture was allowed to warm up to room temperature, stirred for 30 min, followed by the addition of methyl iodide (7.69 g, 54.1 mmol, 2 equiv). The resulting mixture was stirred for 1 h at room temperature while the progress was monitored by LCMS. The reaction was quenched by the addition of water (300 mL) at room temperature and the resulting mixture was extracted with methylene chloride (3×300 mL). The combined organic layers were washed with brine (2×300 mL), dried over anhydrous Na₂SO₄ and concentrated under reduced pressure. The residue was purified by reversed-phase flash chromatography (C18 silica gel; mobile phase, CH₃CN in water, 0% to 100% gradient in 30 min; detector, UV 254/220 nm) to afford tert-butyl N-[2-fluoro-4-(trifluoromethoxy)phenyl]-N-methylcarbamate (6.5 g, 74% yield). LCMS: (ES, m/z): RT=0.954 min, m/z=310.1[M+H]⁺.

Step 3: Into a 250 mL round-bottom flask were added tert-butyl N-[2-fluoro-4-(trifluoromethoxy)phenyl]-N-methylcarbamate (6.50 g, 22.63 mmol, 1.00 equiv) and an anhydrous solution of HCl in 1,4-dioxane (4M, 70.00 mL) at room temperature. The resulting mixture was stirred for 1 h at room temperature. The reaction was monitored by LCMS and concentrated under reduced pressure to afford 2-fluoro-N-methyl-4-(trifluoromethoxy)aniline hydrochloride (5.5 g, 80% yield). LCMS: (ES, m/z): RT=0.872 min, m/z=210.1[M+H]⁺.

Step 4: Into a 250 mL round-bottom flask were added 2-fluoro-N-methyl-4-(trifluoromethoxy)aniline hydrochloride (5.00 g, 23.90 mmol, 1 equiv), dichloromethane (50 mL) and diisopropylethylamine (9.27 g, 71.70 mmol, 3 equiv) at room temperature. Diphosgene (5.68 g, 28.68 mmol, 1.2 equiv) was added at 0° C. and the resulting mixture was allowed to warm up and stirred for 1 h at room temperature under nitrogen atmosphere. The reaction was monitored by LCMS, quenched by the addition of water (300 mL) at room temperature and extracted with ethyl acetate (EtOAc) (3×200 mL). The combined organic layers were washed with water (2×200 mL), dried over anhydrous Na₂SO₄ and concentrated under reduced pressure. The residue was purified by silica gel column chromatography, eluting with petroleum ether/ethyl acetate=1:1 to afford trichloromethyl N-[2-fluoro-4-(trifluoromethoxy)phenyl]-N-methylcarbamate (3.5 g, 40% yield) (“aniline (iv) reagent”). LCMS: (ES, m/z): RT=0.884 min, m/z=367.1[M+H]⁺.

Step 5: Into a 250 mL round-bottom flask were added trichloromethyl N-[2-fluoro-4-(trifluoromethoxy)phenyl]-N-methylcarbamate (“aniline (iv) reagent”) (4.80 g, 12.95 mmol, 1 equiv), a mixture of assumed (1R,2R,4R,5S)-4-methyl-8-(1H-tetrazol-5-yl)-8-azabicyclo[3.2.1]octan-2-amine hydrochloride salt (RRRS-isomer) and (1S,2S,4S,5R)-4-methyl-8-(1H-tetrazol-5-yl)-8-azabicyclo[3.2.1]octan-2-amine hydrochloride salt (SSSR-isomer) (products of Example 5, step 11) (5.40 g, 25.91 mmol, 2 equiv), acetonitrile (50 mL) and diisopropylethylamine (5.02 g, 38.86 mmol, 3 equiv) at room temperature. The resulting mixture was stirred for 1 h at room temperature, then quenched by the addition of water (100 mL) at room temperature and extracted with ethyl acetate (EtOAc) (3×100 mL). The combined organic layers were washed with water (2×100 mL), dried over anhydrous Na₂SO₄ and concentrated under reduced pressure. The residue was purified by reversed-phase flash chromatography (C18 silica gel; mobile phase, CH₃CN in water (0.1% trifluoroacetic acid), 0% to 100% gradient in 30 min; detector, UV 254/220 nm) to afford an assumed mixture of 1-[2-fluoro-4-(trifluoromethoxy)phenyl]-1-methyl-3-[(1R,2R,4R,5S)-4-methyl-8-(1H-1,2,3,4-tetrazol-5-yl)-8-azabicyclo[3.2.1]octan-2-yl]urea trifluoroacetic acid salt (Compound 56A*) and 1-[2-fluoro-4-(trifluoromethoxy)phenyl]-1-methyl-3-[(1S,2S,4S,5R)-4-methyl-8-(1H-1,2,3,4-tetrazol-5-yl)-8-azabicyclo[3.2.1]octan-2-yl]urea trifluoroacetic acid salt (Compound 56D*) (3.80 g, 80% purity). This mixture was further purified by Prep-HPLC (Xselect CSH C18 OBD Column 30*150 mm; 5 m; Mobile Phase A: Water (0.05% trifluoroacetic acid), Mobile Phase B: methanol; Flow rate: 25 mL/min; Gradient: 56% B to 66% B in 10 min; Wave Length: 254/220 nm; RT(min): 11) to afford purified mixture (2.20 g, 38% yield). LCMS: (ES, m/z): RT=0.801 min, m/z=444.1[M+H]⁺.

Step 6: The mixture of step 5 (2.20 g, purity=95%) was separated by Prep-Chiral-HPLC (CHIRAL ART Cellulose-SC, 2*25 cm, 5 μm; Mobile Phase A: Hexanes (0.1% trifluoroacetic acid), Mobile Phase B: ethanol:dichloromethane=1:1; Flow rate: 20 mL/min; Gradient: isocratic 20; Wave Length: 220/254 nm) to afford assumed 1-[2-fluoro-4-(trifluoromethoxy)phenyl]-1-methyl-3-[(1R,2R,4R,5S)-4-methyl-8-(1H-1,2,3,4-tetrazol-5-yl)-8-azabicyclo[3.2.1]octan-2-yl]urea (Compound 56A*) as the first eluting peak (739.7 mg, 29% yield, RT(min)=10.47) and assumed 1-[2-fluoro-4-(trifluoromethoxy)phenyl]-1-methyl-3-[(1S,2S,4S,5R)-4-methyl-8-(1H-1,2,3,4-tetrazol-5-yl)-8-azabicyclo[3.2.1]octan-2-yl]urea (Compound 56D*) as the second eluting peak (660.1 mg, 25% yield, RT(min)=12.91). Stereochemistry arbitrarily assigned.

Compound 56A*: LCMS: (ES, m/z): RT=1.629 min, m/z=444.1 [M+H]. ¹H NMR (400 MHz, Methanol-d₄) δ 7.52 (d, J=8.8 Hz, 1H), 7.33-7.15 (m, 2H), 4.40-4.31 (m, 1H), 4.28-4.05 (m, 2H), 3.31-3.21 (m, 3H), 2.05-1.86 (m, 3H), 1.78-1.62 (m, 3H), 1.53-1.38 (m, 1H), 1.26-1.07 (m, 3H).

Compound 56D*: LCMS: (ES, m/z): RT=1.629 min, m/z=444.1 [M+H]. ¹H NMR (400 MHz, Methanol-d₄) δ 7.52 (d, J=8.8 Hz, 1H), 7.35-7.16 (m, 2H), 4.39-4.29 (m, 1H), 4.26-4.08 (m, 2H), 3.31-3.23 (m, 3H), 2.03-1.82 (m, 3H), 1.77-1.61 (m, 3H), 1.52-1.43 (m, 1H), 1.21-1.05 (m, 3H).

Compounds provided in the below Table A were prepared, or may be prepared, following the General Procedures and Examples as described above. LC-MS data is provided for each compound prepared according to the described Procedure. Dashed (--) line in Table A signifies no data obtained. For purposes of the Examples, including the data provided in the Assay Methods section, “Rac-X” signifies a mixture of 2 or more stereoisomers, e.g., Compounds X′ X″, X″, X″″, XA, XB, XC, XD, XE, XF, XG, and/or XH.

TABLE A
Additional Compounds

| | | LCMS | |
|---|---|---|---|
| # | Compound | (m/z) | Procedure |
| 3A | (R)-3-(1-(1H-tetrazol-5- | 362.1 | Compound 3A was prepared according to Protocol B and |
| | yl)piperidin-3-yl)-1-(4- | | Example 1 using 4-chloro-N-cyclopropylaniline as the aniline |
| | chlorophenyl)-1- | | (iv) reagent and (R)-1-(1H-tetrazol-5-yl)piperidin-3-amine as |
| | cyclopropylurea | | the amine (i) reagent. |
| 3B | (S)-3-(1-(1H-tetrazol-5- | — | Compound 3B may be prepared according to Protocol B and |
| | yl)piperidin-3-yl)-1-(4- | | Example 1 using 4-chloro-N-cyclopropylaniline as the aniline |
| | chlorophenyl)-1- | | (iv) reagent and (S)-1-(1H-tetrazol-5-yl)piperidin-3-amine as |
| | cyclopropylurea | | the amine (i) reagent. |
| Rac-5 | 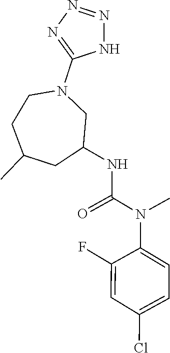 | 382.1 | Rac-5, and Compounds 5A*, 5B*, 5C* and 5D* were prepared following Protocol B and Example 2 using 4-chloro- 2-fluoro-N-methylaniline as the aniline (iv) reagent and 5- methyl-1-(1H-tetrazol-5-yl)azepan-3-amine (i) reagent. Rac-5 was then subjected to prep chiral HPLC (XBridge Shield RP18 OBD Column, 30*150 mm, 5 μm; Mobile Phase A: Water (10 mmol/L NH₄HCO₃), Mobile Phase B: acetonitrile; Flow rate: 60 mL/min; Gradient: 18% B to 28% B in 8 min, 28% B; Wave Length: 254 nm) to provide a mixture of Compounds 5B* and 5C* as the first eluting peak, and a mixture of Compounds 5A* and 5D* as the second eluting peak. Chiral separation conditions: Column: CHIRALPAK IG, 2*25 cm, 5 μm; Mobile Phase A: hexanes (0.1% trifluoroacetic acid), Mobile Phase B: methanol: ethanol = 1:1; Flow rate: 20 mL/min; Gradient: 20% B to 20% B in 11 min; Wave Length: 220/254 nm. |
| | 1-(4-chloro-2- | | |
| | fluorophenyl)-1-methyl-3- | | |
| | (5-methyl-1-(1H-tetrazol-5- | | |
| | yl)azepan-3-yl)urea | | |
| 5A* | 1-(4-chloro-2- | 382.1 | Compound 5A*: Chiral RT(min): 10.07 |
| | fluorophenyl)-1-methyl-3- | | |
| | ((3R,5R)-5-methyl-1-(1H- | | |
| | tetrazol-5-yl)azepan-3- | | |
| | yl)urea | | |
| 5B* | 1-(4-chloro-2- | 382.1 | Compound 5B*: Chiral RT(min): 8.22 |
| | fluorophenyl)-1-methyl-3- | | |
| | ((3S,5R)-5-methyl-1-(1H- | | |
| | tetrazol-5-yl)azepan-3- | | |
| | yl)urea | | |
| 5C* | 1-(4-chloro-2- | 382.1 | Compound 5C*: Chiral RT(min): 10.07 |
| | fluorophenyl)-1-methyl-3- | | |
| | ((3R,5S)-5-methyl-1-(1H- | | |
| | tetrazol-5-yl)azepan-3- | | |
| | yl)urea | | |
| 5D* | 1-(4-chloro-2- | 382.1 | Compound 5D*: Chiral RT(min): 8.22 |
| | fluorophenyl)-1-methyl-3- | | |
| | ((3S,5S)-5-methyl-1-(1H- | | |
| | tetrazol-5-yl)azepan-3- | | |
| | yl)urea | | |
| Rac-7 | 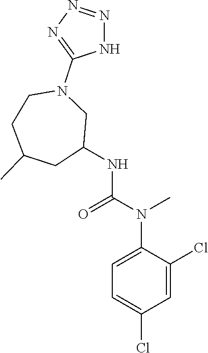 [SMILES: CC1CCN(c2nnn[nH]2)CC(NC(=O)N(C)c2ccc(Cl)cc2Cl)C1] | 398.2 | Rac-7, and Compounds 7A*, 7B*, 7C* and 7D* were prepared following Protocol B and Example 2 using 2,4- dichloro-N-methylaniline as the aniline (iv) reagent and 5- methyl-1-(1H-tetrazol-5-yl)azepan-3-amine as the amine (i) reagent. Rac-7 was then subjected to prep chiral HPLC (XBridge Shield RP18 OBD Column, 30*150 mm, 5 μm; Mobile Phase A: Water (10 mmol/L NH₄HCO₃), Mobile Phase B: acetonitrile; Flow rate: 60 mL/min; Gradient: 20% B to 30% B in 8 min; Wave Length: 254 nm) to provide a mixture of compounds 7B* and 7C* as the first eluting peak, and a mixture of compounds 7A* and 7D* as the second eluting peak. Chiral separation conditions: Column: CHIRALPAK IG, 2*25 cm, 5 μm; Mobile Phase A: hexanes (0.1% trifluoroacetic acid), Mobile Phase B: isopropanol:dichloromethane = 1:1; Flow rate: 20 mL/min; Gradient: 30% B to 30% B in 12.5 min; Wave Length: 220/254 nm. |
| | 1-(2,4-dichlorophenyl)-1- | | |
| | methyl-3-(5-methyl-1-(1H- | | |
| | tetrazol-5-yl)azepan-3- | | |
| | yl)urea | | |
| 7A* | 1-(2,4-dichlorophenyl)-1- | 398.2 | Compound 7A*: Chiral RT(min): 11.43; |
| | methyl-3-((3R,5R)-5- | | |
| | methyl-1-(1H-tetrazol-5- | | |
| | yl)azepan-3-yl)urea | | |
| 7B* | 1-(2,4-dichlorophenyl)-1- | 398.2 | Compound 7B*: Chiral RT(min): 8.98 |
| | methyl-3-((3S,5R)-5- | | |
| | methyl-1-(1H-tetrazol-5- | | |
| | yl)azepan-3-yl)urea | | |
| 7C* | 1-(2,4-dichlorophenyl)-1- | 398.2 | Compound 7C*: Chiral RT(min): 11.43 |
| | methyl-3-((3R,5S)-5- | | |
| | methyl-1-(1H-tetrazol-5- | | |
| | yl)azepan-3-yl)urea | | |
| 7D* | 1-(2,4-dichlorophenyl)-1- | 398.2 | Compound 7D*: Chiral RT(min): 8.98 |
| | methyl-3-((3S,5S)-5- | | |
| | methyl-1-(1H-tetrazol-5- | | |
| | yl)azepan-3-yl)urea | | |
| Rac8- | 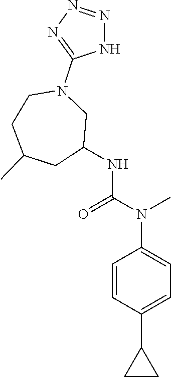 [SMILES: CC1CCN(c2nnn[nH]2)CC(NC(=O)N(C)c2ccc(C3CC3)cc2)C1] | 370.2 | Rac-8 and Compounds 8A*, 8B*, 8C* and 8D* were prepared following Protocol B and Example 2 using 4-cyclopropyl-N- methylaniline as the aniline (iv) reagent and 5-methyl-1-(1H- tetrazol-5-yl)azepan-3-amine as the amine (i) reagent. Rac-8 was then subjected to prep chiral HPLC (XBridge Prep Phenyl OBD Column, 19*250 mm, 5 μm; Mobile Phase A: Water(10 mmol/L NH₄HCO₃), Mobile Phase B: acetonitrile; Flow rate: 25 mL/min; Gradient: 30% B to 40% B in 10 min, 40% B; Wave Length: 254 nm) to provide a mixture of compounds 8B* and 8C* as the first eluting peak, and a mixture of compounds 8A* and 8D* as the second eluting peak. Chiral separation conditions for assumed cis isomers, 8A* and 8D*: Column: CHIRAL ART Cellulose-SC, 2*25 cm. 5 μm: Mobile Phase A: hexanes (0.1% trifluoroacetic acid), Mobile Phase B: isopropanol: dichloromethane = 1:1; Flow rate: 20 mL/min; Gradient: 60% B to 60% B in 16 min; Wave Length: 220/254 nm. Chiral separation conditions for assumed trans isomers, 8B* and 8C*: Column: CHIRALPAK IG, 2*25 cm, 5 μm; Mobile Phase A: hexanes (0.1% trifluoroacetic acid), Mobile Phase B: isopropanol: dichloromethane = 1:1; Flow rate: 20 mL/min; Gradient: 25% B to 25% B in 14 min; Wave Length: 220/254 nm. |
| | 1-(4-cyclopropylphenyl)-1- | | |
| | methyl-3-(5-methyl-1-(1H- | | |
| | tetrazol-5-yl)azepan-3- | | |
| | yl)urea | | |
| 8A* | 1-(4-cyclopropylphenyl)-1- | 370.2 | Compound 8A*: Chiral RT(min): 14.56 |
| | methyl-3-((3R,5R)-5- | | |
| | methyl-1-(1H-tetrazol-5- | | |
| | yl)azepan-3-yl)urea | | |
| 8B* | 1-(4-cyclopropylphenyl)-1- | 370.2 | Compound 8B*: Chiral RT(min): 11.22 |
| | methyl-3-((3S,5R)-5- | | |
| | methyl-1-(1H-tetrazol-5- | | |
| | yl)azepan-3-yl)urea | | |
| 8C* | 1-(4-cyclopropylphenyl)-1- | 370.2 | Compound 8C*: Chiral RT(min): 14.00 |
| | methyl-3-((3R,5S)-5- | | |
| | methyl-1-(1H-tetrazol-5- | | |
| | yl)azepan-3-yl)urea | | |
| 8D* | 1-(4-cyclopropylphenyl)-1- | 370.2 | Compound 8D*: Chiral RT(min): 11.98 |
| | methyl-3-((3S,5S)-5- | | |
| | methyl-1-(1H-tetrazol-5- | | |
| | yl)azepan-3-yl)urea | | |
| Rac-9 | 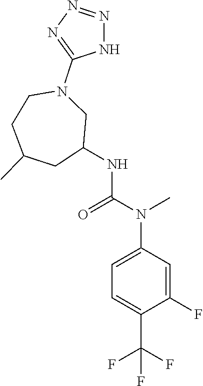 [SMILES: CC1CCN(c2nnn[nH]2)CC(NC(=O)N(C)c2ccc(C(F)(F)F)c(F)c2)C1] | 416.2 | Rac-9 and Compounds 9A*, 9B*, 9C* and 9D* were prepared following Protocol B and Example 2 using 3-fluoro-N-methyl- 4-(trifluoromethyl)aniline as the aniline (iv) reagent and 5- methyl-1-(1H-tetrazol-5-yl)azepan-3-amine as the amine (i) reagent. Rac-9 was then subjected to reverse phase column chromatography (C18 silica gel; mobile phase, acetonitrile in water, 0% to 100% gradient in 30 min; detector, UV 254 nm) to provide a mixture of compounds 9B* and 9C* as the first eluting peak, and a mixture of compounds 9A* and 9D* as the second eluting peak. Chiral separation conditions: Column: CHIRALPAK IG, 2*25 cm, 5 μm Mobile Phase A: hexanes (0.1% trifluoroacetic acid), Mobile Phase B: isopropanol:dichloromethane = 1:1; Flow rate: 20 mL/min; Gradient: 20% B to 20% B in 15.2 min; Wave Length: 220/254 nm. |
| | 1-(3-fluoro-4- | | |
| | (trifluoromethyl)phenyl)-1- | | |
| | methyl-3-(5-methyl-1-(1H- | | |
| | tetrazol-5-yl)azepan-3- | | |
| | yl)urea | | |
| 9A* | 1-(3-fluoro-4- | 416.2 | Compound 9A*: Chiral RT(min): 15.38 |
| | (trifluoromethyl)phenyl)-1- | | |
| | methyl-3-((3R,5R)-5- | | |
| | methyl-1-(1H-tetrazol-5- | | |
| | yl)azepan-3-yl)urea | | |
| 9B* | 1-(3-fluoro-4- | 416.2 | Compound 9B*: Chiral RT(min): 13.369 |
| | (trifluoromethyl)phenyl)-1- | | |
| | methyl-3-((3S,5R)-5- | | |
| | methyl-1-(1H-tetrazol-5- | | |
| | yl)azepan-3-yl)urea | | |
| 9C* | 1-(3-fluoro-4- | 416.2 | Compound 9C*: Chiral RT(min): 21.088 |
| | (trifluoromethyl)phenyl)-1- | | |
| | methyl-3-((3R,5S)-5- | | |
| | methyl-1-(1H-tetrazol-5- | | |
| | yl)azepan-3-yl)urea | | |
| 9D* | 1-(3-fluoro-4- | 416.2 | Compound 9D*: Chiral RT(min): 13.99 |
| | (trifluoromethyl)phenyl)-1- | | |
| | methyl-3-((3S,5S)-5- | | |
| | methyl-1-(1H-tetrazol-5- | | |
| | yl)azepan-3-yl)urea | | |
| Rac-10 | 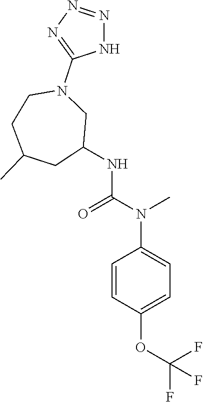 [SMILES: CC1CCN(c2nnn[nH]2)CC(NC(=O)N(C)c2ccc(OC(F)(F)F)cc2)C1] | 414.2 | Rac-10 and Compounds 10A*, 10B*, 10C* and 10D* were prepared following Protocol B and Example 2 using N- methyl-4-(trifluoromethoxy)aniline as the aniline (iv) reagent and 5-methyl-1-(1H-tetrazol-5-yl)azepan-3-amine as the amine (i) reagent. Rac-10 was then subjected to prep chiral HPLC (XBridge Shield RP18 OBD Column, 30*150 mm, 5 μm; Mobile Phase A: Water (10 mmol/L NH₄HCO₃), Mobile Phase B: acetonitrile; Flow rate: 60 mL/min; Gradient: 20% B to 30% B in 10 min, 30% B; Wave Length: 254 nm) to provide a mixture of compounds 10B* and 10C* as the first eluting peak, and a mixture of compounds 10A* and 10D* as the second eluting peak. Chiral separation conditions for assumed cis isomers, 10A* and 10D*: Column: Lux 5 um Cellulose-4, 2.12*25 cm, 5 μm; Mobile Phase A: hexanes (0.2% formic acid), Mobile Phase B: isopropanol; Flow rate: 20 mL/min; Gradient: 25% B to 25% B in 23 min; Wave Length: 220/254 nm. Chiral separation conditions for assumed trans isomers, 10B* and 10C*: Column: Lux 5 um Cellulose-4, 2.12*25 cm, 5 μm; Mobile Phase A: hexanes (0.2% formic acid), Mobile Phase B: isopropanol; Flow rate: 20 mL/min; Gradient: 25% B to 25% B in 24 min; Wave Length: 220/254 nm. |
| | 1-methyl-3-(5-methyl-1- | | |
| | (1H-tetrazol-5-yl)azepan-3- | | |
| | yl)-1-(4- | | |
| | (trifluoromethoxy)phenyl) | | |
| | urea | | |
| 10A | 1-methyl-3-((3R,5R)-5- | 414.2 | Compound 10A*: Chiral RT(min): 20.97 |
| * | methyl-1-(1H-tetrazol-5- | | |
| | yl)azepan-3-yl)-1-(4- | | |
| | (trifluoromethoxy)phenyl) | | |
| | urea | | |
| 10B | 1-methyl-3-((3S,5R)-5- | 414.2 | Compound 10B*: Chiral RT(min): 15.55 |
| * | methyl-1-(1H-tetrazol-5- | | |
| | yl)azepan-3-yl)-1-(4- | | |
| | (trifluoromethoxy)phenyl) | | |
| | urea | | |
| 10C | 1-methyl-3-((3R,5S)-5- | 414.2 | Compound 10C*: Chiral RT(min): 21.15 |
| * | methyl-1-(1H-tetrazol-5- | | |
| | yl)azepan-3-yl)-1-(4- | | |
| | (trifluoromethoxy)phenyl) | | |
| | urea | | |
| 10D | 1-methyl-3-((3S,5S)-5- | 414.2 | Compound 10D*: Chiral RT(min): 16.79 |
| * | methyl-1-(1H-tetrazol-5- | | |
| | yl)azepan-3-yl)-1-(4- | | |
| | (trifluoromethoxy)phenyl) | | |
| | urea | | |
| Rac-11 | 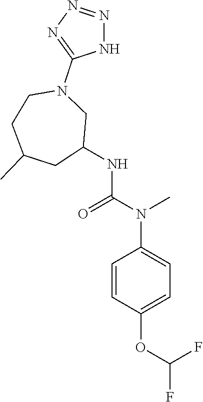 [SMILES: CC1CCN(c2nnn[nH]2)CC(NC(=O)N(C)c2ccc(OC(F)F)cc2)C1] | 396.2 | Rac-11 and Compounds 11A*, 11B*, 11C* and 11D* were prepared following Protocol B and Example 2 using 4- (difluoromethoxy)-N-methylaniline as the aniline (iv) reagent and 5-methyl-1-(1H-tetrazol-5-yl)azepan-3-amine as the amine (i) reagent. Rac-11 was then subjected to reverse phase column chromatography (C18 silica gel; mobile phase, acetonitrile in water, 0% to 100% gradient in 30 min; detector, UV 254 nm) to provide a mixture of compounds 11B* and 11C* as the first eluting peak, and a mixture of compounds 11A* and 11D* as the second eluting peak. Chiral separation conditions for assumed cis isomers, 11A* and 11D*: Column: CHIRALPAK IG, 2*25 cm, 5 μm; Mobile Phase A: hexanes (0.1% trifluoroacetic acid), Mobile Phase B: ethanol; Flow rate: 20 mL/min; Gradient: 15% B to 15% B in 18 min; Wave Length: 220/254 nm. Chiral separation conditions for assumed trans isomers, 11B* and 11C*: Column: CHIRALPAK IG, 2*25 cm, 5 μm; Mobile Phase A: hexanes (0.1% trifluoroacetic acid), Mobile Phase B: isopropanol; Flow rate: 20 mL/min; Gradient: 20% B to 20% B in 16.6 min; Wave Length: 220/254 nm. |
| | 1-(4- | | |
| | (difluoromethoxy)phenyl)- | | |
| | 1-methyl-3-(5-methyl-1- | | |
| | (1H-tetrazol-5-yl)azepan-3- | | |
| | yl)urea | | |
| 11A | 1-(4- | 396.2 | Compound 11A*: Chiral RT(min): 11.22 |
| * | (difluoromethoxy)phenyl)- | | |
| | 1-methyl-3-((3R,5R)-5- | | |
| | methyl-1-(1H-tetrazol-5- | | |
| | yl)azepan-3-yl)urea | | |
| 11B | 1-(4- | 396.2 | Compound 11B*: Chiral RT(min): 16.04 |
| * | (difluoromethoxy)phenyl)- | | |
| | 1-methyl-3-((3S,5R)-5- | | |
| | methyl-1-(1H-tetrazol-5- | | |
| | yl)azepan-3-yl)urea | | |
| 11C | 1-(4- | 396.2 | Compound 11C*: Chiral RT(min): 12.54 |
| * | (difluoromethoxy)phenyl)- | | |
| | 1-methyl-3-((3R,5S)-5- | | |
| | methyl-1-(1H-tetrazol-5- | | |
| | yl)azepan-3-yl)urea | | |
| 11D | 1-(4- | 396.2 | Compound 11D*: Chiral RT(min): 9.13 |
| * | (difluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-12 | 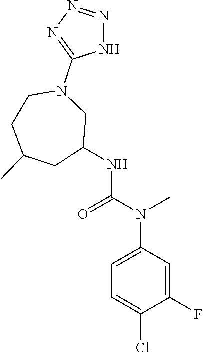 [SMILES: CC1CCN(c2nnn[nH]2)CC(NC(=O)N(C)c2ccc(Cl)c(F)c2)C1] | 382.1 | Rac-12 and Compounds 12A*, 12B*, 12C* and 12D* were prepared following Protocol B and Example 2 using 3-fluoro- 4-chloro-N-methylaniline as the aniline (iv) reagent and 5- methyl-1-(1H-tetrazol-5-yl)azepan-3-amine as the amine (i) reagent. Rac-12 was then subjected to prep chiral HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 21% B to 29% B in 8 min, 29% B; Wave Length: 220/254 nm) to provide a mixture of compounds 12B* and 12C* as the first eluting peak, and a mixture of compounds 12A* and 12D* as the second eluting peak. Chiral separation conditions for assumed cis isomers, 12A* and 12D*: Column: CHIRALPAK AD-H, 2*25 cm, 5 μm; Mobile Phase A: hexanes (0.1% trifluoroacetic acid), Mobile Phase B: isopropanol; Flow rate: 20 mL/min; Gradient: 15% B to 15% B in 18.5 min; Wave Length: 220/254 nm. Chiral separation conditions for assumed trans isomers, 12B* and 12C*: Column: Lux 5 um Cellulose-4, 2.12*25 cm, 5 μm Mobile Phase A: hexanes (0.1% trifluoroacetic acid), Mobile Phase B: isopropanol; Flow rate: 20 mL/min; Gradient: 50% B to 50% B in 14 min; Wave Length: 220/254 nm. |
| | 1-(4-chloro-3- | | |
| | fluorophenyl)-1-methyl-3- | | |
| | (5-methyl-1-(1H-tetrazol-5- | | |
| | yl)azepan-3-yl)urea | | |
| 12A | 1-(4-chloro-3- | 382.1 | Compound 12A*: Chiral RT(min): 12.62 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 12B | 1-(4-chloro-3- | 382.1 | Compound 12B*: Chiral RT(min): 8.11 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 12C | 1-(4-chloro-3- | 382.1 | Compound 12C*: Chiral RT(min): 10.26 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 12D | 1-(4-chloro-3- | 382.1 | Compound 12D*: Chiral RT(min): 9.28 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-13 | 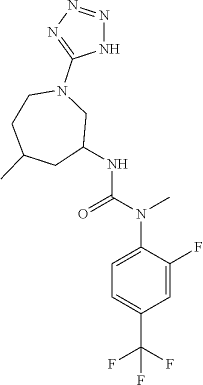 [SMILES: CC1CCN(c2nnn[nH]2)CC(NC(=O)N(C)c2ccc(C(F)(F)F)cc2F)C1] | 416.2 | Rac-13 and Compounds 13A*, 13B*, 13C* and 13D* were prepared following Protocol B and Example 2 using 2-fluoro- N-methyl-4-(trifluoromethyl)aniline as the aniline (iv) reagent and 5-methyl-1-(1H-tetrazol-5-yl)azepan-3-amine as the amine (i) reagent. Rac-13 was then subjected to reverse phase column chromatography (C18 silica gel; mobile phase, acetonitrile in water, 0% to 100% gradient in 30 min; detector, UV 254 nm) to provide a mixture of compounds 13B* and 13C* as the first eluting peak, and a mixture of compounds 13A* and 13D* as the second eluting peak. Chiral separation conditions for assumed cis isomers, 13A* and 13D*: Column: CHIRALPAK IG, 2*25 cm, 5 μm; Mobile Phase A: hexanes (0.5% 2M NH₃-methanol), Mobile Phase B: ethanol: dichloromethane = 1:1; Flow rate: 20 mL/min; Gradient: 15% B to 15% B in 15 min; Wave Length: 254/220 nm. Chiral separation conditions for assumed trans isomers, 13B* and 13C*: Column: Lux 5 um Cellulose-4, 2.12*25 cm, 5 μm Mobile Phase A: hexanes (0.2% formic acid), Mobile Phase B: isopropanol; Flow rate: 20 mL/min; Gradient: 30% B to 30% B in 14.5 min; Wave Length: 254/220 nm. |
| | 1-(2-fluoro-4- | | |
| | (trifluoromethyl)phenyl)-1- | | |
| | methyl-3-(5-methyl-1-(1H- | | |
| | tetrazol-5-yl)azepan-3- | | |
| | yl)urea | | |
| 13A | 1-(2-fluoro-4- | 416.2 | Compound 13A*: Chiral RT(min): 12.81 |
| * | (trifluoromethyl)phenyl)-1- | | |
| | methyl-3-((3R,5R)-5- | | |
| | methyl-1-(1H-tetrazol-5- | | |
| | yl)azepan-3-yl)urea | | |
| 13B | 1-(2-fluoro-4- | 416.2 | Compound 13B*: Chiral RT(min): 10.01 |
| * | (trifluoromethyl)phenyl)-1- | | |
| | methyl-3-((3S,5R)-5- | | |
| | methyl-1-(1H-tetrazol-5- | | |
| | yl)azepan-3-yl)urea | | |
| 13C | 1-(2-fluoro-4- | 416.2 | Compound 13C*: Chiral RT(min): 12.23 |
| * | (trifluoromethyl)phenyl)-1- | | |
| | methyl-3-((3R,5S)-5- | | |
| | methyl-1-(1H-tetrazol-5- | | |
| | yl)azepan-3-yl)urea | | |
| 13D | 1-(2-fluoro-4- | 416.2 | Compound 13D*: Chiral RT(min): 9.04 |
| * | (trifluoromethyl)phenyl)-1- | | |
| | methyl-3-((3S,5S)-5- | | |
| | methyl-1-(1H-tetrazol-5- | | |
| | yl)azepan-3-yl)urea | | |
| Rac-14 | 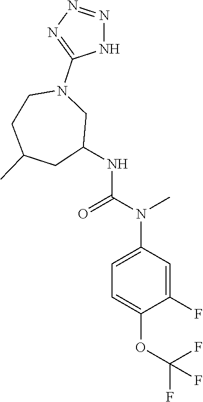 [SMILES: CC1CCN(c2nnn[nH]2)CC(NC(=O)N(C)c2ccc(OC(F)(F)F)c(F)c2)C1] | 432.1 | Rac-14 and Compounds 14A*, 14B*, 14C* and 14D* were prepared following Protocol B and Example 2 using 3-fluoro- N-methyl-4-(trifluoromethoxy)aniline as the aniline (iv) reagent and 5-methyl-1-(1H-tetrazol-5-yl)azepan-3-amine as the amine (i) reagent. Rac-14 was then subjected to prep chiral HPLC (XSelect CSH OBD Column 30*150 mm, 5 mm; Mobile Phase A: Water (0.05% trifluoroacetic acid), Mobile Phase B: acetonitrile; Flow rate: 60 mL/min mL/min; Gradient: 39% B to 49% B in 9 min; Wave Length: 254 nm/220 nm) to provide a mixture of compounds 14B* and 14C* as the first eluting peak, and a mixture of compounds 14A* and 14D* as the second eluting peak. Chiral separation conditions for assumed cis isomers, 14A* and 14D*: CHIRAL ART Amylose-C NEO, 2*25 cm, 5 μm; Mobile Phase A: hexanes (0.1% trifluoroacetic acid), Mobile Phase B: isopropanol; Flow rate: 20 mL/min; Gradient: 15% B to 15% B in 9.5 min; Wave Length: 220/254 nm. Chiral separation conditions for assumed trans isomers, 14B* and 14C*: Column: Lux 5 um Cellulose-4, 2.12*25 cm, 5 μm; Mobile Phase A: hexanes (0.1% trifluoroacetic acid), Mobile Phase B: isopropanol; Flow rate: 20 mL/min; Gradient: 30% B to 30% B in 14 min; Wave Length: 220/254 nm. |
| | 1-(3-fluoro-4- | | |
| | (trifluoromethoxy)phenyl)- | | |
| | 1-methyl-3-(5-methyl-1- | | |
| | (1H-tetrazol-5-yl)azepan-3- | | |
| | yl)urea | | |
| 14A | 1-(3-fluoro-4- | 432.1 | Compound 14A*: Chiral RT(min): 8.747 |
| * | (trifluoromethoxy)phenyl)- | | |
| | 1-methyl-3-((3R,5R)-5- | | |
| | methyl-1-(1H-tetrazol-5- | | |
| | yl)azepan-3-yl)urea | | |
| 14B | 1-(3-fluoro-4- | 432.1 | Compound 14B*: Chiral RT(min): 12.59 |
| * | (trifluoromethoxy)phenyl)- | | |
| | 1-methyl-3-((3S,5R)-5- | | |
| | methyl-1-(1H-tetrazol-5- | | |
| | yl)azepan-3-yl)urea | | |
| 14C | 1-(3-fluoro-4- | 432.1 | Compound 14C*: Chiral RT(min): 5.43 |
| * | (trifluoromethoxy)phenyl)- | | |
| | 1-methyl-3-((3R,5S)-5- | | |
| | methyl-1-(1H-tetrazol-5- | | |
| | yl)azepan-3-yl)urea | | |
| 14D | 1-(3-fluoro-4- | 432.1 | Compound 14D*: Chiral RT(min): 6.42 |
| * | (trifluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-15 | 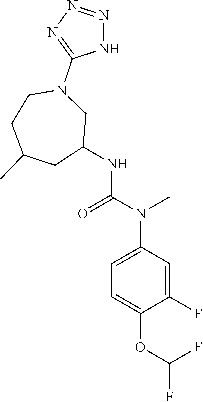 [SMILES: CC1CCN(c2nnn[nH]2)CC(NC(=O)N(C)c2ccc(OC(F)F)c(F)c2)C1] | 414.1 | Rac-15 and Compounds 15A*, 15B*, 15C* and 15D* were prepared following Protocol B and Example 2 using 4- (difluoromethoxy)-3-fluoro-N-methylaniline as the aniline (iv) reagent and 5-methyl-1-(1H-tetrazol-5-yl)azepan-3-amine as the amine (i) reagent. Rac-15 was then subjected to prep chiral HPLC (XBridge Prep Phenyl Column, 19*250 mm, 5 μm; Mobile Phase A: Water (0.1% trifluoroacetic acid), Mobile Phase B: acetonitrile; Flow rate: 25 mL/min mL/min; Gradient: 40% B to 50% B in 9 min; Wave Length: 254 nm/220 nm) to provide a mixture of compounds 15B* and 15C* as the first eluting peak, and a mixture of compounds 15A* and 15D* as the second eluting peak. Chiral separation conditions for assumed cis isomers, 15A* and 15D*: Column: Lux 5 um Cellulose-3, 2.12*25 cm, 5 μm Mobile Phase A: hexanes (0.1% trifluoroacetic acid), Mobile Phase B: ethanol; Flow rate: 20 mL/min; Gradient: 10% B to 10% B in 14 min; Wave Length: 220/254 nm. Chiral separation conditions for assumed trans isomers, 15B* and 15C*: Column: Lux 5 um Cellulose-4, 2.12*25 cm, 5 μm; Mobile Phase A: hexanes (0.1% trifluoroacetic acid), Mobile Phase B: ethanol; Flow rate: 20 mL/min; Gradient: 20% B to 20% B in 13 min; Wave Length: 220/254 nm. |
| | 1-(4-(difluoromethoxy)-3- | | |
| | fluorophenyl)-1-methyl-3- | | |
| | (5-methyl-1-(1H-tetrazol-5- | | |
| | yl)azepan-3-yl)urea | | |
| 15A | 1-(4-(difluoromethoxy)-3- | 414.1 | Compound 15A*: RT(min): 14.44 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 15B | 1-(4-(difluoromethoxy)-3- | 414.1 | Compound 15B*: Chiral RT(min): 10.35 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 15C | 1-(4-(difluoromethoxy)-3- | 414.1 | Compound 15C*: Chiral RT(min): 12.08 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 15D | 1-(4-(difluoromethoxy)-3- | 414.1 | Compound 15D*: Chiral RT(min): 12.02 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-16 | 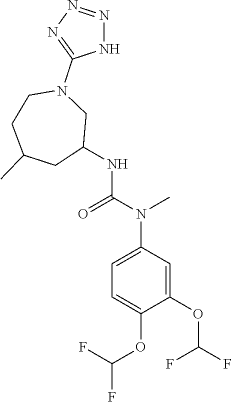 [SMILES: CC1CCN(c2nnn[nH]2)CC(NC(=O)N(C)c2ccc(OC(F)F)c(OC(F)F)c2)C1] | 462.1 | Rac-16 and Compounds 16A*, 16B*, 16C* and 16D* were prepared following Protocol B and Example 2 using 3,4- bis(difluoromethoxy)-N-methylaniline as the aniline (iv) reagent and 5-methyl-1-(1H-tetrazol-5-yl)azepan-3-amine as the amine (i) reagent. Rac-16 was then subjected to prep chiral HPLC (XSelect CSH OBD Column 30*150 mm, 5 umn; Mobile Phase A: Water(0.05% trifluoroacetic acid), Mobile Phase B: acetonitrile; Flow rate: 60 mL/min mL/min; Gradient: 36% B to 46% B in 9 min; Wave Length: 254 nm/220 nm) to provide a mixture of compounds 16B* and 16C* as the first eluting peak, and a mixture of compounds 16A* and 16D* as the second eluting peak. Chiral separation conditions for assumed cis isomers, 16A* and 16D*: CHIRALPAK IG, 2*25 cm, 5 μm Mobile Phase A: hexanes (0.1% trifluoroacetic acid),, Mobile Phase B: ethanol:dichloromethane = 1:1; Flow rate: 20 mL/min; Gradient: 20% B to 20% B in 9.5 min; Wave Length: 220/254 nm. Chiral separation conditions for assumed trans isomers, 16B* and 16C*: Column: (R, R)-WHELK-01-Kromasil, 5*25 cm, 5 μm Mobile Phase A: hexanes (0.1% trifluoroacetic acid),, Mobile Phase B: isopropanol; Flow rate: 20 mL/min; Gradient: 20% B to 20% B in 20 min; Wave Length: 220/254 nm. |
| | 1-(3,4- | | |
| | bis(difluoromethoxy)phenyl)- | | |
| | 1-methyl-3-(5-methyl-1- | | |
| | (1H-tetrazol-5-yl)azepan-3- | | |
| | yl)urea | | |
| 16A | 1-(3,4- | 462.1 | Compound 16A*: Chiral RT(min): 8.88 |
| * | bis(difluoromethoxy)phenyl)- | | |
| | 1-methyl-3-((3R,5R)-5- | | |
| | methyl-1-(1H-tetrazol-5- | | |
| | yl)azepan-3-yl)urea | | |
| 16B | 1-(3,4- | 462.1 | Compound 16B*: Chiral RT(min): 18.30 |
| * | bis(difluoromethoxy)phenyl)- | | |
| | 1-methyl-3-((3S,5R)-5- | | |
| | methyl-1-(1H-tetrazol-5- | | |
| | yl)azepan-3-yl)urea | | |
| 16C | 1-(3,4- | 462.1 | Compound 16C*: Chiral RT(min): 14.65 |
| * | bis(difluoromethoxy)phenyl)- | | |
| | 1-methyl-3-((3R,5S)-5- | | |
| | methyl-1-(1H-tetrazol-5- | | |
| | yl)azepan-3-yl)urea | | |
| 16D | 1-(3,4- | 462.1 | Compound 16D*: Chiral RT(min): 7.04 |
| * | bis(difluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-17 | 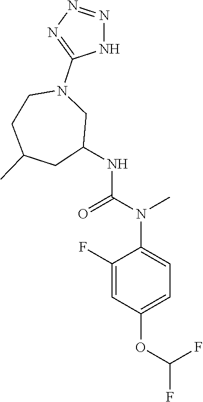 [SMILES: CC1CCN(c2nnn[nH]2)CC(NC(=O)N(C)c2ccc(OC(F)F)cc2F)C1] | 414.2 | Rac-17 and Compounds 17A*, 17B*, 17C* and 17D* were prepared following Protocol B and Example 2 using 4- (difluoromethoxy)-2-fluoro-N-methylaniline as the aniline (iv) reagent and 5-methyl-1-(1H-tetrazol-5-yl)azepan-3-amine as the amine (i) reagent. Rac-17 was then subjected to prep chiral HPLC (XSelect CSH OBD Column 30*150 mm, 5 umn; Mobile Phase A: Water(0.05% trifluoroacetic acid), Mobile Phase B: acetonitrile; Flow rate: 60 mL/min mL/min; Gradient: 30% B to 40% to 44% B in 14 min; Wave Length: 254 nm/220 nm) to provide a mixture of compounds 17B* and 17C* as the first eluting peak, and a mixture of compounds 17A* and 17D* as the second eluting peak. Chiral separation conditions for assumed cis isomers, 17A* and 17D*: Column: Lux 5 um Cellulose-2 2.12*25 cm, 5 μm; Mobile Phase A: Hexanes (0.1% trifluoroacetic acid), Mobile Phase B: isopropanol; Flow rate: 20 mL/min; Gradient: 20% B to 20% B in 28 min; Wave Length: 220/254 nm. Chiral separation conditions for assumed trans isomers, 17B* and 17C*: Column: CHIRALPAK IG, 2*25 cm, 5 μm; Mobile Phase A: Hexanes (0.5% 2M ammonia in methanol), Mobile Phase B: Ethanol:dichloromethane = 1:1; Flow rate: 20 mL/min; Gradient: 15% B to 15% B in 16 min; Wave Length: 254/220 nm. |
| | 1-(4-(difluoromethoxy)-2- | | |
| | fluorophenyl)-1-methyl-3- | | |
| | (5-methyl-1-(1H-tetrazol-5- | | |
| | yl)azepan-3-yl)urea | | |
| 17A | 1-(4-(difluoromethoxy)-2- | 414.2 | Compound 17A*: Chiral RT(min): 24.72 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 17B | 1-(4-(difluoromethoxy)-2- | 414.2 | Compound 17B*: Chiral RT(min): 11.44 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 17C | 1-(4-(difluoromethoxy)-2- | 414.2 | Compound 17C*: Chiral RT(min): 14.66 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 17D | 1-(4-(difluoromethoxy)-2- | 414.2 | Compound 17D*: Chiral RT(min): 15.64 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-18 | 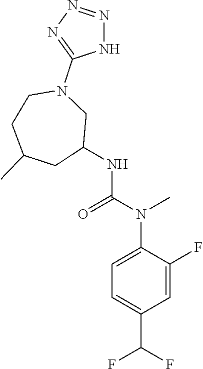 [SMILES: CC1CCN(c2nnn[nH]2)CC(NC(=O)N(C)c2ccc(C(F)F)cc2F)C1] | 398.2 | Rac-18 and Compounds 18A*, 18B*, 18C* and 18D* were prepared following Protocol B and Example 2 using 4- (difluoromethyl)-2-fluoro-N-methylaniline as the aniline (iv) reagent and 5-methyl-1-(1H-tetrazol-5-yl)azepan-3-amine as the amine (i) reagent. Rac-18 was then subjected to reverse phase column chromatography (C18 silica gel; mobile phase, acetonitrile in water, 0% to 100% gradient in 30 min; detector, UV 254 nm) to provide a mixture of compounds 18B* and 18C* as the first eluting peak, and a mixture of compounds 18A* and 18D* as the second eluting peak. Chiral separation conditions for assumed cis isomers, 18A* and 18D*: Column: CHIRALPAK IG, 2*25 cm, 5 μm; Mobile Phase A: hexanes (0.1% trifluoroacetic acid), Mobile Phase B: isopropanol:dichloromethane = 1:1; Flow rate: 20 mL/min; Gradient: 25% B to 25% B in 15 min; Wave Length: 220/254 nm. Chiral separation conditions for assumed trans isomers, 18B* and 18C*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 30% B to 30% B in 17 min; Wave Length: 220/254 nm. |
| | 1-(4-(difluoromethyl)-2- | | |
| | fluorophenyl)-1-methyl-3- | | |
| | (5-methyl-1-(1H-tetrazol-5- | | |
| | yl)azepan-3-yl)urea | | |
| 18A | 1-(4-(difluoromethyl)-2- | 398.2 | Compound 18A*: Chiral RT(min): 12.44 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 18B | 1-(4-(difluoromethyl)-2- | 398.2 | Compound 18B*: Chiral RT(min): 11.13 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 18C | 1-(4-(difluoromethyl)-2- | 398.2 | Compound 18C*: Chiral RT(min): 15.07 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 18D | 1-(4-(difluoromethyl)-2- | 398.2 | Compound 18D*: Chiral RT(min): 8.83 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-19 | 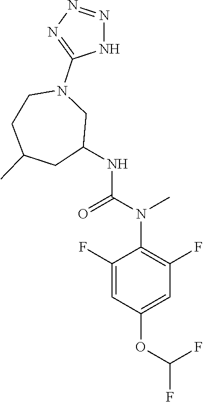 [SMILES: CC1CCN(c2nnn[nH]2)CC(NC(=O)N(C)c2c(F)cc(OC(F)F)cc2F)C1] | 432.1 | Rac-19 and Compounds 19A*, 19B*, 19C* and 19D* were prepared following Protocol B and Example 2 using 4- (difluoromethoxy)-2,6-difluoro-N-methylaniline as the aniline (iv) reagent and 5-methyl-1-(1H-tetrazol-5-yl)azepan-3-amine as the amine (i) reagent. Rac-19 was then subjected to reverse phase column chromatography (C18 silica gel; mobile phase, acetonitrile in water, 0% to 100% gradient in 30 min; detector, UV 254 nm) to provide a mixture of compounds 19B* and 19C* as the first eluting peak, and a mixture of compounds 19A* and 19D* as the second eluting peak. Chiral separation conditions for assumed cis isomers, 19A* and 19D*: Column: CHIRALPAK IG, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 20% B to 20% B in 15 min; Wave Length: 220/254 nm. Chiral separation conditions for assumed trans isomers, 19B* and 19C*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 20% B to 20% B in 19 min; Wave Length: 220/254 nm. |
| | 1-(4-(difluoromethoxy)-2,6- | | |
| | difluorophenyl)-1-methyl- | | |
| | 3-(5-methyl-1-(1H-tetrazol- | | |
| | 5-yl)azepan-3-yl)urea | | |
| 19A | 1-(4-(difluoromethoxy)-2,6- | 432.1 | Compound 19A*: Chiral RT(min): 12.8 |
| * | difluorophenyl)-1-methyl- | | |
| | 3-((3R,5R)-5-methyl-1- | | |
| | (1H-tetrazol-5-yl)azepan-3- | | |
| | yl)urea | | |
| 19B | 1-(4-(difluoromethoxy)-2,6- | 432.1 | Compound 19B*: Chiral RT(min): 11.01 |
| * | difluorophenyl)-1-methyl- | | |
| | 3-((3S,5R)-5-methyl-1-(1H- | | |
| | tetrazol-5-yl)azepan-3- | | |
| | yl)urea | | |
| 19C | 1-(4-(difluoromethoxy)-2,6- | 432.1 | Compound 19C*: Chiral RT(min): 16.73 |
| * | difluorophenyl)-1-methyl- | | |
| | 3-((3R,5S)-5-methyl-1-(1H- | | |
| | tetrazol-5-yl)azepan-3- | | |
| | yl)urea | | |
| 19D | 1-(4-(difluoromethoxy)-2,6- | 432.1 | Compound 19D*: Chiral RT(min): 9.01 |
| * | difluorophenyl)-1-methyl- | | |
| | 3-((3S,5S)-5-methyl-1-(1H- | | |
| | tetrazol-5-yl)azepan-3- | | |
| | yl)urea | | |
| Rac-20 | 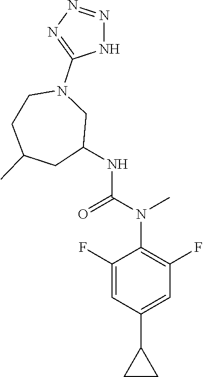 [SMILES: CC1CCN(c2nnn[nH]2)CC(NC(=O)N(C)c2c(F)cc(C3CC3)cc2F)C1] | 406.3 | Rac-20 and Compounds 20A*, 20B*, 20C* and 20D* were prepared following Protocol B and Example 2 using 4- cyclopropyl-2,6-difluoro-N-methylaniline as the aniline (iv) reagent and 5-methyl-1-(1H-tetrazol-5-yl)azepan-3-amine as the amine (i) reagent. Rac-20 was then subjected to reverse phase column chromatography (C18 silica gel; mobile phase, acetonitrile in water, 0% to 100% gradient in 30 min; detector, UV 254 nm) to provide a mixture of compounds 20B* and 20C* as the first eluting peak, and a mixture of compounds 20A* and 20D* as the second eluting peak. Chiral separation conditions for assumed cis isomers, 20A* and 20D*: Column: CHIRAL ART Amylose-C NEO, 2*25 cm, 5 μm; Mobile Phase A: Hexanes (0.1% trifluoroacetic acid), Mobile Phase B: isopropanol; Flow rate: 20 mL/min; Gradient: 15% B to 15% B in 13 min; Wave Length: 220/254 nm. Chiral separation conditions for assumed trans isomers, 20B* and 20C*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 50% B to 50% B in 14 min; Wave Length: 220/254 nm. |
| | 1-(4-cyclopropyl-2,6- | | |
| | difluorophenyl)-1-methyl- | | |
| | 3-(5-methyl-1-(1H-tetrazol- | | |
| | 5-yl)azepan-3-yl)urea | | |
| 20A | 1-(4-cyclopropyl-2,6- | 406.3 | Compound 20A*: Chiral RT(min): 11.68 |
| * | difluorophenyl)-1-methyl- | | |
| | 3-((3R,5R)-5-methyl-1- | | |
| | (1H-tetrazol-5-yl)azepan-3- | | |
| | yl)urea | | |
| 20B | 1-(4-cyclopropyl-2,6- | 406.3 | Compound 20B*: Chiral RT(min): 6.91 |
| * | difluorophenyl)-1-methyl- | | |
| | 3-((3S,5R)-5-methyl-1-(1H- | | |
| | tetrazol-5-yl)azepan-3- | | |
| | yl)urea | | |
| 20C | 1-(4-cyclopropyl-2,6- | 406.3 | Compound 20C*: Chiral RT(min): 11 |
| * | difluorophenyl)-1-methyl- | | |
| | 3-((3R,5S)-5-methyl-1-(1H- | | |
| | tetrazol-5-yl)azepan-3- | | |
| | yl)urea | | |
| 20D | 1-(4-cyclopropyl-2,6- | 406.3 | Compound 20D*: Chiral RT(min): 6.69 |
| * | difluorophenyl)-1-methyl- | | |
| | 3-((3S,5S)-5-methyl-1-(1H- | | |
| | tetrazol-5-yl)azepan-3- | | |
| | yl)urea | | |
| Rac-21 | 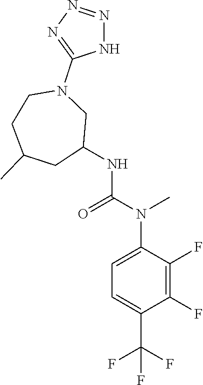 [SMILES: CC1CCN(c2nnn[nH]2)CC(NC(=O)N(C)c2ccc(C(F)(F)F)c(F)c2F)C1] | 434.2 | Rac-21 and Compounds 21A*, 21B*, 21C* and 21D* were prepared following Protocol B and Example 2 using 2,3- difluoro-N-methyl-4-(trifluoromethyl)aniline as the aniline (iv) reagent and 5-methyl-1-(1H-tetrazol-5-yl)azepan-3-amine as the amine (i) reagent. Rac-21 was then subjected to prep chiral HPLC (XSelect CSH Prep C18 OBD Column, 19*250 mm, 5 μm; mobile phase, Water (0.05% trifluoroacetic acid) and acetonitrile; Detector, UV 254 nm) to provide a mixture of compounds 21B* and 21C* as the first eluting peak, and a mixture of compounds 21A* and 21D* as the second eluting peak. Chiral separation conditions for assumed cis isomers, 21A* and 21D*: Column: CHIRALPAK IG, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 15% B to 15% B in 28 min; Wave Length: 220/254 nm. Chiral separation conditions for assumed trans isomers, 21B* and 21C*: Column: CHIRAL ART Amylose-SA, 2*25 cm, 5 μm; Mobile Phase A: methyl tertbutyl ether (MtBE) (0.1% trifluoroacetic acid), Mobile Phase B: isopropanol:dichloromethane = 1:1; Flow rate: 20 mL/min; Gradient: 10% B to 10% B in 25 min; Wave Length: 220/254 nm. |
| | 1-(2,3-difluoro-4- | | |
| | (trifluoromethyl)phenyl)-1- | | |
| | methyl-3-(5-methyl-1-(1H- | | |
| | tetrazol-5-yl)azepan-3- | | |
| | yl)urea | | |
| 21A | 1-(2,3-difluoro-4- | 434.2 | Compound 21A*: Chiral RT(min): 23.08 |
| * | (trifluoromethyl)phenyl)-1- | | |
| | methyl-3-((3R,5R)-5- | | |
| | methyl-1-(1H-tetrazol-5- | | |
| | yl)azepan-3-yl)urea | | |
| 21B | 1-(2,3-difluoro-4- | 434.2 | Compound 21B*: Chiral RT(min): 19.66 |
| * | (trifluoromethyl)phenyl)-1- | | |
| | methyl-3-((3S,5R)-5- | | |
| | methyl-1-(1H-tetrazol-5- | | |
| | yl)azepan-3-yl)urea | | |
| 21C | 1-(2,3-difluoro-4- | 434.2 | Compound 21C*: Chiral RT(min): 21.88 |
| * | (trifluoromethyl)phenyl)-1- | | |
| | methyl-3-((3R,5S)-5- | | |
| | methyl-1-(1H-tetrazol-5- | | |
| | yl)azepan-3-yl)urea | | |
| 21D | 1-(2,3-difluoro-4- | 434.2 | Compound 21D*: Chiral RT(min): 14.35 |
| * | (trifluoromethyl)phenyl)-1- | | |
| | methyl-3-((3S,5S)-5- | | |
| | methyl-1-(1H-tetrazol-5- | | |
| | yl)azepan-3-yl)urea | | |
| Rac-22 | 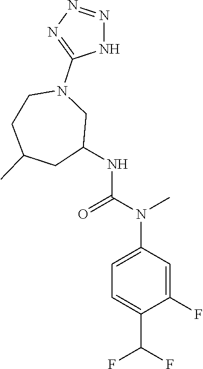 [SMILES: CC1CCN(c2nnn[nH]2)CC(NC(=O)N(C)c2ccc(C(F)F)c(F)c2)C1] | 398.1 | Rac-22 and Compounds 22A*, 22B*, 22C* and 22D* were prepared following Protocol B and Example 2 using 4- (difluoromethyl)-3-fluoro-N-methylaniline hydrochloride as the aniline (iv) reagent and 5-methyl-1-(1H-tetrazol-5- yl)azepan-3-amine as the amine (i) reagent. Rac-22 was then subjected to prep chiral HPLC (XSelect CSH OBD Column 30*150 mm, 5 umn; Mobile Phase A: Water (0.1% formic acid), Mobile Phase B: acetonitrile; Flow rate: 60 mL/min; Gradient: 50% B to 60% B in 9 min; Wave Length: 254 nm/220 nm) to provide a mixture of compounds 22B* and 22C* as the first eluting peak, and a mixture of compounds 22A* and 22D* as the second eluting peak. Chiral separation conditions for assumed cis isomers, 22A* and 22D*: Column: CHIRALPAK IG, 2*25 cm, 5 um; Mobile Phase A: Hexanes (0.1% trifluoroacetic acid), Mobile Phase B: isopropanol:dichloromethane = 1:1; Flow rate: 20 mL/min; Gradient: 20% B to 20% B in 25 min; Wave Length: 220/254 nm. Chiral separation conditions for assumed trans isomers, 22B* and 22C*: Column: CHIRALPAK IE, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 25% B to 25% B in 28 min; Wave Length: 220/254 nm. |
| | 1-(4-(difluoromethyl)-3- | | |
| | fluorophenyl)-1-methyl-3- | | |
| | (5-methyl-1-(1H-tetrazol-5- | | |
| | yl)azepan-3-yl)urea | | |
| 22A | 1-(4-(difluoromethyl)-3- | 398.1 | Compound 22A*: Chiral RT(min): 22.37 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 22B | 1-(4-(difluoromethyl)-3- | 398.1 | Compound 22B*: Chiral RT(min): 22.24 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 22C | 1-(4-(difluoromethyl)-3- | 398.1 | Compound 22C*: Chiral RT(min): 26.22 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 22D | 1-(4-(difluoromethyl)-3- | 398.1 | Compound 22D*: Chiral RT(min): 17.11 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-23 | 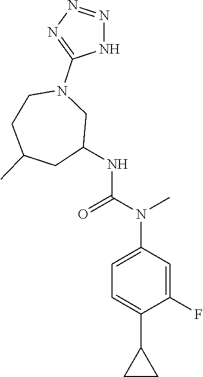 [SMILES: CC1CCN(c2nnn[nH]2)CC(NC(=O)N(C)c2ccc(C3CC3)c(F)c2)C1] | 388.1 | Rac-23 and Compounds 23A*, 23B*, 23C* and 23D* were prepared following Protocol B and Example 2 using 4- cyclopropyl-3-fluoro-N-methylaniline as the aniline (iv) reagent and 5-methyl-1-(1H-tetrazol-5-yl)azepan-3-amine as the amine (i) reagent. Rac-23 was then subjected to prep chiral HPLC (XSelect CSH OBD Column 30*150 mm, 5 umn; Mobile Phase A: water (0.05% trifluoroacetic acid), Mobile Phase B: acetonitrile; Flow rate: 60 mL/min mL/min; Gradient: 36% B to 46% B in 9 min; Wave Length: 254 nm/220 nm) to provide a mixture of compounds 23B* and 23C* as the first eluting peak, and a mixture of compounds 23A* and 23D* as the second eluting peak. Chiral separation conditions for assumed cis isomers, 23A* and 23D*: Column: CHIRALPAK AS-H, 2*25 cm, 5 μm; Mobile Phase A: Hexanes (0.1% trifluoroacetic acid), Mobile Phase B: isopropanol; Flow rate: 20 mL/min; Gradient: 15% B to 15% B in 22 min; Wave Length: 220/254 nm. Chiral separation conditions for assumed trans isomers, 23B* and 23C*: Column: CHIRALPAK AS-H, 2*25 cm, 5 μm; Mobile Phase A: Hexanes (0.1% trifluoroacetic acid), Mobile Phase B: Isopropanol; Flow rate: 20 mL/min; Gradient: 30% B to 30% B in 15.5 min; Wave Length: 220/254 nm. |
| | 1-(4-cyclopropyl-3- | | |
| | fluorophenyl)-1-methyl-3- | | |
| | (5-methyl-1-(1H-tetrazol-5- | | |
| | yl)azepan-3-yl)urea | | |
| 23A | 1-(4-cyclopropyl-3- | 388.1 | Compound 23A*: Chiral RT(min): 14.44 |
| * | fluorophenyl)-1-methyl-3- | | |
| | ((3R,5R)-5-methyl-1-(1H- | | |
| | tetrazol-5-yl)azepan-3- | | |
| | yl)urea | | |
| 23B | 1-(4-cyclopropyl-3- | 388.1 | Compound 23B*: Chiral RT(min): 9.26 |
| * | fluorophenyl)-1-methyl-3- | | |
| | ((3S,5R)-5-methyl-1-(1H- | | |
| | tetrazol-5-yl)azepan-3- | | |
| | yl)urea | | |
| 23C | 1-(4-cyclopropyl-3- | 388.1 | Compound 23C*: Chiral RT(min): 12.96 |
| * | fluorophenyl)-1-methyl-3- | | |
| | ((3R,5S)-5-methyl-1-(1H- | | |
| | tetrazol-5-yl)azepan-3- | | |
| | yl)urea | | |
| 23D | 1-(4-cyclopropyl-3- | 388.1 | Compound 23D*: Chiral RT(min): 18.59 |
| * | fluorophenyl)-1-methyl-3- | | |
| | ((3S,5S)-5-methyl-1-(1H- | | |
| | tetrazol-5-yl)azepan-3- | | |
| | yl)urea | | |
| Rac-24 | 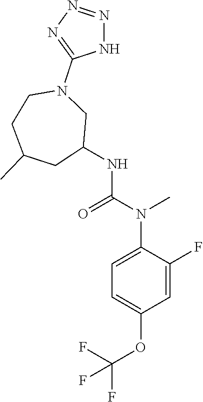 [SMILES: CC1CCN(c2nnn[nH]2)CC(NC(=O)N(C)c2ccc(OC(F)(F)F)cc2F)C1] | 432.1 | Rac-24 and Compounds 24A*, 24B*, 24C* and 24D* were prepared following Protocol B and Example 2 using 2-fluoro- N-methyl-4-(trifluoromethoxy)aniline as the aniline (iv) reagent and 5-methyl-1-(1H-tetrazol-5-yl)azepan-3-amine as the amine (i) reagent. Rac-24 was then subjected to prep chiral HPLC (XBridge Prep Phenyl Column, 19*250 mm, 5 μm; Mobile Phase A: Water (0.1% trifluoroacetic acid), Mobile Phase B: acetonitrile; Flow rate: 25 mL/min; Gradient: 40% B to 50% B in 9 min; Wave Length: 254 nm/220 nm) to provide a mixture of compounds 24B* and 24C* as the first eluting peak, and a mixture of compounds 24A* and 24D* as the second eluting peak. Chiral separation conditions for assumed cis isomers, 24A* and 24D*: Column: CHIRALPAK ID, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 20% B to 20% B in 14 min; Wave Length: 220/254 nm. Chiral separation conditions for assumed trans isomers, 24B* and 24C*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 30% B to 30% B in 14 min; Wave Length: 220/254 nm. Compound 24A*: Chiral RT(min): 12.11 |
| | 1-(2-fluoro-4- | | |
| | (trifluoromethoxy)phenyl)- | | |
| | 1-methyl-3-(5-methyl-1- | | |
| | (1H-tetrazol-5-yl)azepan-3- | | |
| | yl)urea | | |
| 24A | 1-(2-fluoro-4- | 432.1 | Compound 24A*: Chiral RT(min): 12.11 |
| * | (trifluoromethoxy)phenyl)- | | |
| | 1-methyl-3-((3R,5R)-5- | | |
| | methyl-1-(1H-tetrazol-5- | | |
| | yl)azepan-3-yl)urea | | |
| 24B | 1-(2-fluoro-4- | 432.1 | Compound 24B*: Chiral RT(min): 9.49 |
| * | (trifluoromethoxy)phenyl)- | | |
| | 1-methyl-3-((3S,5R)-5- | | |
| | methyl-1-(1H-tetrazol-5- | | |
| | yl)azepan-3-yl)urea | | |
| 240 | 1-(2-fluoro-4- | 432.1 | Compound 24C*: Chiral RT(min): 12.09 |
| * | (trifluoromethoxy)phenyl)- | | |
| | 1-methyl-3-((3R,5S)-5- | | |
| | methyl-1-(1H-tetrazol-5- | | |
| | yl)azepan-3-yl)urea | | |
| 24D | 1-(2-fluoro-4- | 432.1 | Compound 24D*: Chiral RT(min): 9.64 |
| * | (trifluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-25 | 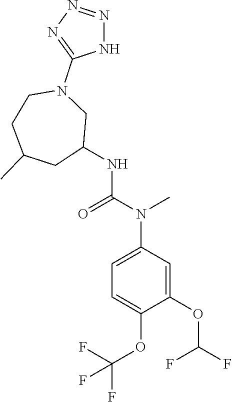 [SMILES: CC1CCN(c2nnn[nH]2)CC(NC(=O)N(C)c2ccc(OC(F)(F)F)c(OC(F)F)c2)C1] | 480.2 | Rac-25 and Compounds 25A*, 25B*, 25C* and 25D* were prepared following Protocol B and Example 2 using 3- (difluoromethoxy)-N-methyl-4-(trifluoromethoxy)aniline as the aniline (iv) reagent and 5-methyl-1-(1H-tetrazol-5- yl)azepan-3-amine as the amine (i) reagent. Rac-25 was then subjected to prep chiral HPLC (XSelect CSH OBD Column 30*150 mm, 5 umn; Mobile Phase A: Water (0.05% trifluoroacetic acid), Mobile Phase B: acetonitrile; Flow rate: 60 mL/min mL/min; Gradient: 3% B to 18% B in 10 min; Wave Length: 254 nm/220 nm) to provide a mixture of compounds 25B* and 25C* as the first eluting peak, and a mixture of compounds 25A* and 25D* as the second eluting peak. Chiral separation conditions for assumed cis isomers, 25A* and 25D*: Column: CHIRALPAK AS-H, 2*25 cm, 5 μm; Mobile Phase A: Hexanes (0.1% trifluoroacetic acid), Mobile Phase B: isopropanol; Flow rate: 20 mL/min; Gradient: 15% B to 15% B in 22 min; Wave Length: 220/254 nm. Chiral separation conditions for assumed trans isomers, 25B* and 25C*: Column: Lux 5 um Cellulose-4, 2.12*25 cm, 5 μm; Mobile Phase A: Hexanes (0.1% trifluoroacetic acid), Mobile Phase B: Ethanol:dichloromethane = 1:1; Flow rate: 20 mL/min; Gradient: 20% B to 20% B in 18 min; Wave Length: 220/254 nm. |
| | 1-(3-(difluoromethoxy)-4- | | |
| | (trifluoromethoxy)phenyl)- | | |
| | 1-methyl-3-(5-methyl-1- | | |
| | (1H-tetrazol-5-yl)azepan-3- | | |
| | yl)urea | | |
| 25A | 1-(3-(difluoromethoxy)-4- | 480.2 | Compound 25A*: Chiral RT(min): 18.59 |
| * | (trifluoromethoxy)phenyl)- | | |
| | 1-methyl-3-((3R,5R)-5- | | |
| | methyl-1-(1H-tetrazol-5- | | |
| | yl)azepan-3-yl)urea | | |
| 25B | 1-(3-(difluoromethoxy)-4- | 480.2 | Compound 25B*: Chiral RT(min): 12.78 |
| * | (trifluoromethoxy)phenyl)- | | |
| | 1-methyl-3-((3S,5R)-5- | | |
| | methyl-1-(1H-tetrazol-5- | | |
| | yl)azepan-3-yl)urea | | |
| 25C | 1-(3-(difluoromethoxy)-4- | 480.2 | Compound 25C*: Chiral RT(min): 16.07 |
| * | (trifluoromethoxy)phenyl)- | | |
| | 1-methyl-3-((3R,5S)-5- | | |
| | methyl-1-(1H-tetrazol-5- | | |
| | yl)azepan-3-yl)urea | | |
| 25D | 1-(3-(difluoromethoxy)-4- | 480.2 | Compound 25D*: Chiral RT(min): 14.44 |
| * | (trifluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-26 | 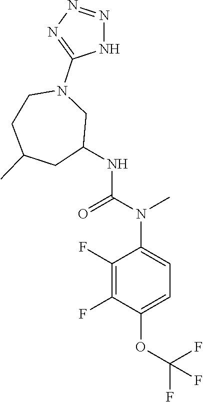 [SMILES: CC1CCN(c2nnn[nH]2)CC(NC(=O)N(C)c2ccc(OC(F)(F)F)c(F)c2F)C1] | 450.2 | Rac-26 and Compounds 26A*, 26B*, 26C* and 26D* were prepared following Protocol B and Example 2 using 2,3- difluoro-N-methyl-4-(trifluoromethoxy)aniline as the aniline (iv) reagent and 5-methyl-1-(1H-tetrazol-5-yl)azepan-3-amine as the amine (i) reagent. Rac-26 was then subjected to reverse phase column chromatography (C18 silica gel; mobile phase, acetonitrile in water, 0% to 100% gradient in 30 min; detector, UV 254 nm) to provide a mixture of compounds 26B* and 26C* as the first eluting peak, and a mixture of compounds 26A* and 26D* as the second eluting peak. Chiral separation conditions for assumed cis isomers, 26A* and 26D*: Column: CHIRALPAK IG, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 20% B to 20% B in 13 min; Wave Length: 220/254 nm. Chiral separation conditions for assumed trans isomers, 26B* and 26C*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 10% B to 10% B in 25 min; Wave Length: 220/254 nm. |
| | 1-(2,3-difluoro-4- | | |
| | (trifluoromethoxy)phenyl)- | | |
| | 1-methyl-3-(5-methyl-1- | | |
| | (1H-tetrazol-5-yl)azepan-3- | | |
| | yl)urea | | |
| 26A | 1-(2,3-difluoro-4- | 450.2 | Compound 26A*: Chiral RT(min): 10.16 |
| * | (trifluoromethoxy)phenyl)- | | |
| | 1-methyl-3-((3R,5R)-5- | | |
| | methyl-1-(1H-tetrazol-5- | | |
| | yl)azepan-3-yl)urea | | |
| 26B | 1-(2,3-difluoro-4- | 450.2 | Compound 26B*: Chiral RT(min): 12.58 |
| * | (trifluoromethoxy)phenyl)- | | |
| | 1-methyl-3-((3S,5R)-5- | | |
| | methyl-1-(1H-tetrazol-5- | | |
| | yl)azepan-3-yl)urea | | |
| 26C | 1-(2,3-difluoro-4- | 450.2 | Compound 26C*: Chiral RT(min): 15.79 |
| * | (trifluoromethoxy)phenyl)- | | |
| | 1-methyl-3-((3R,5S)-5- | | |
| | methyl-1-(1H-tetrazol-5- | | |
| | yl)azepan-3-yl)urea | | |
| 26D | 1-(2,3-difluoro-4- | 450.2 | Compound 26D*: Chiral RT(min): 6.72 |
| * | (trifluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-27 | 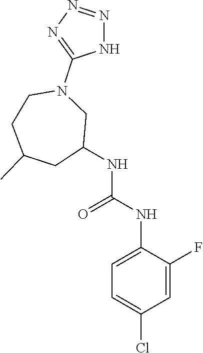 [SMILES: CC1CCN(c2nnn[nH]2)CC(NC(=O)Nc2ccc(Cl)cc2F)C1] 1-(4-chloro-2-fluorophenyl)-3-(5- methyl-1-(1H-tetrazol-5-yl)azepan- 3-yl)urea | 368.2 | Rac-27 and Compounds 27A*, 27B*, 27C* and 27D* were prepared following Protocol B and Example 2 using 2-fluoro- 4-chloro-N-methylaniline as the aniline (iv) reagent and 5- methyl-1-(1H-tetrazol-5-yl)azepan-3-amine as the amine (i) reagent. Rac-27 was then subjected to prep chiral HPLC (XSelect CSH C₁₈ OBD Column 30*150 mm 5 μm, n; Mobile Phase A: acetonitrile, Mobile Phase B: Water(0.05% trifluoroacetic acid); Flow rate: 60 mL/min; Gradient: 36% B to 46% B in 8 min, 46% B to 46% B in 12 min, 46% B; Wave Length: 254/220 nm) to provide a mixture of compounds 27B* and 27C* as the first eluting peak, and a mixture of compounds 27A* and 27D* as the second eluting peak. Chiral separation conditions for assumed cis isomers, 27A* and 27D*: Column: CHIRALPAK IF, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 15% B to 15% B in 14.5 min; Wave Length: 220/254 nm. Chiral separation conditions for assumed trans isomers, 27B* and 27C*: Column: CHIRAL ART Cellulose-SC, 2*25 cm, 5 um; Mobile Phase A: Hexanes (0.1% trifluoroacetic acid), Mobile Phase B: Ethanol:dichloromethane = 1:1; Flow rate: 20 mL/min; Gradient: 20% B to 20% B in 15 min; Wave Length: 220/254 nm. |
| 27A | 1-(4-chloro-2- | 368.2 | Compound 27A*: Chiral RT(min): 13.71 |
| * | fluorophenyl)-3-((3R,5R)- | | |
| | 5-methyl-1-(1H-tetrazol-5- | | |
| | yl)azepan-3-yl)urea | | |
| 27B | 1-(4-chloro-2- | 368.2 | Compound 27B*: Chiral RT(min): 10.46 |
| * | fluorophenyl)-3-((3S,5R)-5- | | |
| | methyl-1-(1H-tetrazol-5- | | |
| | yl)azepan-3-yl)urea | | |
| 27C | 1-(4-chloro-2- | 368.2 | Compound 27C*: Chiral RT(min): 12.89 |
| * | fluorophenyl)-3-((3R,5S)-5- | | |
| | methyl-1-(1H-tetrazol-5- | | |
| | yl)azepan-3-yl)urea | | |
| 27D | 1-(4-chloro-2- | 368.2 | Compound 27D*: Chiral RT(min): 11.39 |
| * | fluorophenyl)-3-((3S,5S)-5- | | |
| | methyl-1-(1H-tetrazol-5- | | |
| | yl)azepan-3-yl)urea | | |
| Rac-28 | 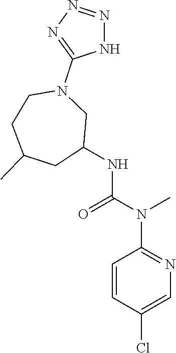 [SMILES: CC1CCN(c2nnn[nH]2)CC(NC(=O)N(C)c2ccc(Cl)cn2)C1] | 365.1 | Rac-28 and Compounds 28A*, 28B*, 28C* and 28D* were prepared following Protocol B and Example 2 using 5-chloro- N-methylpyridin-2-amine as the aniline (iv) reagent and 5- methyl-1-(1H-tetrazol-5-yl)azepan-3-amine as the amine (i) reagent. Rac-28 was then subjected to reverse phase column chromatography (C18 silica gel; mobile phase, acetonitrile in water, 0% to 100% gradient in 30 min; detector, UV 254 nm) to provide a mixture of compounds 28B* and 28C* as the first eluting peak, and a mixture of compounds 28A* and 28D* as the second eluting peak. Chiral separation conditions for assumed cis isomers, 28A* and 28D*: Column: CHIRALPAK IG, 2*25 cm, 5 μm; Mobile Phase A: Hexanes (0.1% trifluoroacetic acid), Mobile Phase B: isopropanol; Flow rate: 20 mL/min; Gradient: 35% B to 35% B in 22 min; Wave Length: 220/254 nm. Chiral separation conditions for assumed trans isomers, 28B* and 28C*: Column: CHIRALPAK IG, 2*25 cm, 5 μm; Mobile Phase A: Hexanes (0.1% trifluoroacetic acid), Mobile Phase B: methanol:dichloromethane = 1:1; Flow rate: 20 mL/min; Gradient: 20% B to 20% B in 18 min; Wave Length: 220/254 nm. |
| | 1-(5-chloropyridin-2-yl)-1- | | |
| | methyl-3-(5-methyl-1-(1H- | | |
| | tetrazol-5-yl)azepan-3- | | |
| | yl)urea | | |
| 28A | 1-(5-chloropyridin-2-yl)-1- | 365.1 | Compound 28A*: Chiral RT(min): 15.24 |
| * | methyl-3-((3R,5R)-5- | | |
| | methyl-1-(1H-tetrazol-5- | | |
| | yl)azepan-3-yl)urea | | |
| 28B | 1-(5-chloropyridin-2-yl)-1- | 365.1 | Compound 28B*: Chiral RT(min): 13.76 |
| * | methyl-3-((3S,5R)-5- | | |
| | methyl-1-(1H-tetrazol-5- | | |
| | yl)azepan-3-yl)urea | | |
| 28C | 1-(5-chloropyridin-2-yl)-1- | 365.1 | Compound 28C*: Chiral RT(min): 16.71 |
| * | methyl-3-((3R,5S)-5- | | |
| | methyl-1-(1H-tetrazol-5- | | |
| | yl)azepan-3-yl)urea | | |
| 28D | 1-(5-chloropyridin-2-yl)-1- | 365.1 | Compound 28D*: Chiral RT(min): 19.51 |
| * | methyl-3-((3S,5S)-5- | | |
| | methyl-1-(1H-tetrazol-5- | | |
| | yl)azepan-3-yl)urea | | |
| Rac-29 | 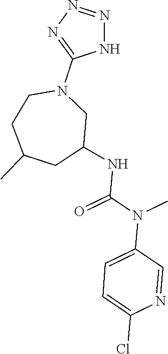 | 365.1 | Rac-29 and Compounds 29A*, 29B*, 29C* and 29D* were prepared following Protocol B and Example 2 using 6-chloro- N-methylpyridin-3-amine as the aniline (iv) reagent and 5- methyl-1-(1H-tetrazol-5-yl)azepan-3-amine as the amine (i) reagent. Rac-29 was then subjected to prep chiral HPLC (XBridge Shield RP18 OBD Column, 30*150 mm, 5 μm; mobile phase, Water (10 mmol/L NH₄HCO₃) and acetonitrile (11% MeCN up to 21% MeCN in 10 min); Detector, UV 254 nm) to provide a mixture of compounds 29B* and 29C* as the first eluting peak, and a mixture of compounds 29A* and 29D* as the second eluting peak. Chiral separation conditions for assumed cis isomers, 29A* and 29D*: Column: CHIRALPAK IG, 2*25 cm, 5 μm; Mobile Phase A: Hexanes (0.1% trifluoroacetic acid), Mobile Phase B: isopropanol; Flow rate: 20 mL/min; Gradient: 30% B to 30% B in 19 min; Wave Length: 220/254 nm. Chiral separation conditions for assumed trans isomers, 29B* and 29C*: Column: CHIRALPAK IG, 2*25 cm, 5 μm; Mobile Phase A: Hexanes (0.1% trifluoroacetic acid), Mobile Phase B: Methanol:dichloromethane = 1:1; Flow rate: 20 mL/min; Gradient: 25% B to 25% B in 14.5 min; Wave Length: 254/220 nm. |
| | 1-(6-chloropyridin-3-yl)-1- | | |
| | methyl-3-(5-methyl-1-(1H- | | |
| | tetrazol-5-yl)azepan-3- | | |
| | yl)urea | | |
| 29A | 1-(6-chloropyridin-3-yl)-1- | 365.1 | Compound 29A*: Chiral RT(min): 17.27 |
| * | methyl-3-((3R,5R)-5- | | |
| | methyl-1-(1H-tetrazol-5- | | |
| | yl)azepan-3-yl)urea | | |
| 29B | 1-(6-chloropyridin-3-yl)-1- | 365.1 | Compound 29B*: Chiral RT(min): 9.12 |
| * | methyl-3-((3S,5R)-5- | | |
| | methyl-1-(1H-tetrazol-5- | | |
| | yl)azepan-3-yl)urea | | |
| 29C | 1-(6-chloropyridin-3-yl)-1- | 365.1 | Compound 29C*: Chiral RT(min): 11.66 |
| * | methyl-3-((3R,5S)-5- | | |
| | methyl-1-(1H-tetrazol-5- | | |
| | yl)azepan-3-yl)urea | | |
| 29D | 1-(6-chloropyridin-3-yl)-1- | 365.1 | Compound 29D*: Chiral RT(min): 13.40 |
| * | methyl-3-((3S,5S)-5- | | |
| | methyl-1-(1H-tetrazol-5- | | |
| | yl)azepan-3-yl)urea | | |
| Rac-30 | 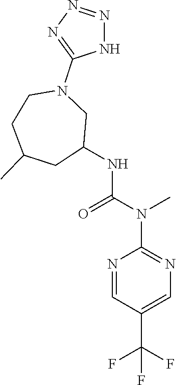 [SMILES: CC1CCN(c2nnn[nH]2)CC(NC(=O)N(C)c2ncc(C(F)(F)F)cn2)C1] | 400.2 | Rac-30 and Compounds 30A*, 30B*, 30C* and 30D* were prepared following Protocol B and Example 2 using N- methyl-5-(trifluoromethyl)pyrimidin-2-amine as the aniline (iv) reagent and 5-methyl-1-(1H-tetrazol-5-yl)azepan-3-amine as the amine (i) reagent. Rac-30 was then subjected to prep chiral HPLC (XSelect CSH Prep C18 OBD Column, 19*250 mm, 5 μm; mobile phase, Water (0.05% trifluoroacetic acid) and methanol (hold 72% methanol in 8 min); Detector, UV 254 nm) to provide a mixture of compounds 30B* and 30C* as the first eluting peak, and a mixture of compounds 30A* and 30D* as the second eluting peak. Chiral separation conditions: Column, CHIRAL ART Cellulose-SB, 2*25 cm, 5 μm; mobile phase, hexanes (0.1% trifluoroacetic acid) and isopropanol:dichloromethane = 1:1 (hold 25% isopropanol:dichloromethane = 1:1 in 8 min). |
| | 1-methyl-3-(5-methyl-1- | | |
| | (1H-tetrazol-5-yl)azepan-3- | | |
| | yl)-1-(5- | | |
| | (trifluoromethyl)pyrimidin- | | |
| | 2-yl)urea | | |
| 30A | 1-methyl-3-((3R,5R)-5- | 400.2 | Compound 30A*: Chiral RT(min): 8.46 |
| * | methyl-1-(1H-tetrazol-5- | | |
| | yl)azepan-3-yl)-1-(5- | | |
| | (trifluoromethyl)pyrimidin- | | |
| | 2-yl)urea | | |
| 30B | 1-methyl-3-((3S,5R)-5- | 400.2 | Compound 30B*: Chiral RT(min): 5.87 |
| * | methyl-1-(1H-tetrazol-5- | | |
| | yl)azepan-3-yl)-1-(5- | | |
| | (trifluoromethyl)pyrimidin- | | |
| | 2-yl)urea | | |
| 30C | 1-methyl-3-((3R,5S)-5- | 400.2 | Compound 30C*: Chiral RT(min): 6.92 |
| * | methyl-1-(1H-tetrazol-5- | | |
| | yl)azepan-3-yl)-1-(5- | | |
| | (trifluoromethyl)pyrimidin- | | |
| | 2-yl)urea | | |
| 30D | 1-methyl-3-((3S,5S)-5- | 400.2 | Compound 30D*: Chiral RT(min): 10.01 |
| * | methyl-1-(1H-tetrazol-5- | | |
| | yl)azepan-3-yl)-1-(5- | | |
| | (trifluoromethyl)pyrimidin- | | |
| | 2-yl)urea | | |
| Rac-31 | 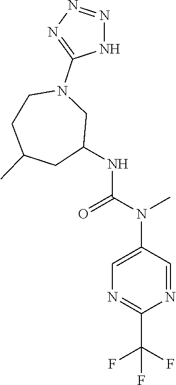 [SMILES: CC1CCN(c2nnn[nH]2)CC(NC(=O)N(C)c2cnc(C(F)(F)F)nc2)C1] | 400.2 | Rac-31 and Compounds 31A*, 31B*, 31C* and 31D* were prepared following Protocol B and Example 2 using N- methyl-2-(trifluoromethyl)pyrimidin-5-amine as the aniline (iv) reagent and 5-methyl-1-(1H-tetrazol-5-yl)azepan-3-amine as the amine (i) reagent. Rac-31 was then subjected to prep chiral HPLC (CHIRALPAK IG-3, 4.6*50 mm, 3 um; Mobile Phase A: hexanes (0.1% trifluoroacetic acid): (methanol: dichloromethane = 1:1) = 80:20; Flow rate: 1 mL/min; Gradient: 0% B to 0% B); Detector, UV 254 nm) to provide a mixture of compounds 31B* and 31C* as the first eluting peak, and a mixture of compounds 31A* and 31D* as the second eluting peak. Chiral separation conditions for assumed cis isomers, 31A* and 31D*: Column: CHIRAL ART Cellulose-SB, 4.6*100 mm, 3um; hexanes (0.1% trifluoroacetic acid): (isopropanol:dichloromethane = 1:1) = 70:30; Flow rate: 1 mL/min; Wave Length: 254/220 nm. Chiral separation conditions for assumed trans isomers, 31B* and 31C*: Column: CHIRALPAK IG-3, 4.6*50 mm, 3 um; Mobile Phase A: hexanes (0.1% trifluoroacetic acid): (methanol:dichloromethane = 1:1) = 80:20; Flow rate: 1 mL/min; Wave Length: 254/220 nm. |
| | 1-methyl-3-(5-methyl-1- | | |
| | (1H-tetrazol-5-yl)azepan-3- | | |
| | yl)-1-(2- | | |
| | (trifluoromethyl)pyrimidin- | | |
| | 5-yl)urea | | |
| 31A | 1-methyl-3-((3R,5R)-5- | 400.2 | Compound 31A*: Chiral RT(min): 13.34 |
| * | methyl-1-(1H-tetrazol-5- | | |
| | yl)azepan-3-yl)-1-(2- | | |
| | (trifluoromethyl)pyrimidin- | | |
| | 5-yl)urea | | |
| 31B | 1-methyl-3-((3S,5R)-5- | 400.2 | Compound 31B*: Chiral RT(min): 7.60 |
| * | methyl-1-(1H-tetrazol-5- | | |
| | yl)azepan-3-yl)-1-(2- | | |
| | (trifluoromethyl)pyrimidin- | | |
| | 5-yl)urea | | |
| 31C | 1-methyl-3-((3R,5S)-5- | 400.2 | Compound 31C*: Chiral RT(min): 10.66 |
| * | methyl-1-(1H-tetrazol-5- | | |
| | yl)azepan-3-yl)-1-(2- | | |
| | (trifluoromethyl)pyrimidin- | | |
| | 5-yl)urea | | |
| 31D | 1-methyl-3-((3S,5S)-5- | 400.2 | Compound 31D*: Chiral RT(min): 10.84 |
| * | methyl-1-(1H-tetrazol-5- | | |
| | yl)azepan-3-yl)-1-(2- | | |
| | (trifluoromethyl)pyrimidin- | | |
| | 5-yl)urea | | |
| Rac-32 | 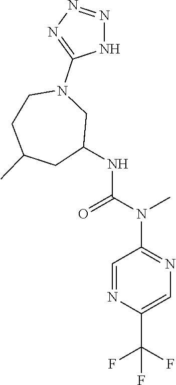 [SMILES: CC1CCN(c2nnn[nH]2)CC(NC(=O)N(C)c2cnc(C(F)(F)F)cn2)C1] | 400.2 | Rac-32 and Compounds 32A*, 32B*, 32C* and 32D* were prepared following Protocol B and Example 2 using N- methyl-5-(trifluoromethyl)pyrazin-2-amine as the aniline (iv) reagent and 5-methyl-1-(1H-tetrazol-5-yl)azepan-3-amine as the amine (i) reagent. Rac-32 was then subjected to prep chiral HPLC (XSelect CSH C18 OBD Column 30*150 mm 5 um; mobile phase, acetonitrile and water (0.05% trifluoroacetic acid) (32% water (0.05% trifluoroacetic acid) up to 40% in 8 min, up to 45% in 4 min); Detector, UV 254 nm) to provide a mixture of compounds 32B* and 32C* as the first eluting peak, and a mixture of compounds 32A* and 32D* as the second eluting peak. Chiral separation conditions for assumed cis isomers, 32A* and 32D*: Column: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 30% B to 30% B in 11 min; Wave Length: 220/254 nm. Chiral separation conditions for assumed trans isomers, 32B* and 32C*: Column: Lux 5 um Cellulose-4, 2.12*25 cm, 5 μm Mobile Phase A: Hexanes (0.1% trifluoroacetic acid), Mobile Phase B: methanol:ethanol = 1:1; Flow rate: 20 mL/min; Gradient: 30% B to 30% B in 11 min; Wave Length: 220/254 nm. |
| | 1-methyl-3-(5-methyl-1- | | |
| | (1H-tetrazol-5-yl)azepan-3- | | |
| | yl)-1-(5- | | |
| | (trifluoromethyl)pyrazin-2- | | |
| | yl)urea | | |
| 32A | 1-methyl-3-((3R,5R)-5- | 400.2 | Compound 32A*: Chiral RT(min): 10.36 |
| * | methyl-1-(1H-tetrazol-5- | | |
| | yl)azepan-3-yl)-1-(5- | | |
| | (trifluoromethyl)pyrazin-2- | | |
| | yl)urea | | |
| 32B | 1-methyl-3-((3S,5R)-5- | 400.2 | Compound 32B*: Chiral RT(min): 9.54 |
| * | methyl-1-(1H-tetrazol-5- | | |
| | yl)azepan-3-yl)-1-(5- | | |
| | (trifluoromethyl)pyrazin-2- | | |
| | yl)urea | | |
| 32C | 1-methyl-3-((3R,5S)-5- | 400.2 | Compound 32C*: Chiral RT(min): 10.63 |
| * | methyl-1-(1H-tetrazol-5- | | |
| | yl)azepan-3-yl)-1-(5- | | |
| | (trifluoromethyl)pyrazin-2- | | |
| | yl)urea | | |
| 32D | 1-methyl-3-((3S,5S)-5- | 400.2 | Compound 32D*: Chiral RT(min): 8.75 |
| * | methyl-1-(1H-tetrazol-5- | | |
| | yl)azepan-3-yl)-1-(5- | | |
| | (trifluoromethyl)pyrazin-2- | | |
| | yl)urea | | |
| Rac-33 | 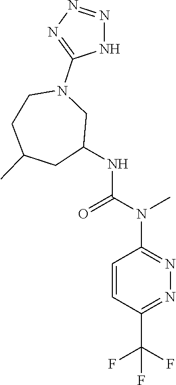 [SMILES: CC1CCN(c2nnn[nH]2)CC(NC(=O)N(C)c2ccc(C(F)(F)F)nn2)C1] | 400.2 | Rac-33 and Compounds 33A*, 33B*, 33C* and 33D* were prepared following Protocol B and Example 2 using N- methyl-6-(trifluoromethyl)pyridazin-3-amine as the aniline (iv) reagent and 5-methyl-1-(1H-tetrazol-5-yl)azepan-3-amine as the amine (i) reagent. Rac-33 was then subjected to prep chiral HPLC (XBridge Shield RP18 OBD Column, 30*150 mm, 5 μm; Mobile Phase A: Water (10 mmol/L NH₄HCO₃), Mobile Phase B: acetonitrile; Flow rate: 60 mL/min; Gradient: 18% B to 30% B in 8 min, 30% B; Wave Length: 254 nm) to provide a mixture of compounds 33B* and 33C* as the first eluting peak, and a mixture of compounds 33A* and 33D* as the second eluting peak. Chiral separation conditions: Column: CHIRAL ART Cellulose-SB, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 25% B to 25% B in 17 min; Wave Length: 220/254 nm. |
| | 1-methyl-3-(5-methyl-1- | | |
| | (1H-tetrazol-5-yl)azepan-3- | | |
| | yl)-1-(6- | | |
| | (trifluoromethyl)pyridazin- | | |
| | 3-yl)urea | | |
| 33A | 1-methyl-3-((3R,5R)-5- | 400.2 | Compound 33A*: Chiral RT(min): 13.55 |
| * | methyl-1-(1H-tetrazol-5- | | |
| | yl)azepan-3-yl)-1-(6- | | |
| | (trifluoromethyl)pyridazin- | | |
| | 3-yl)urea | | |
| 33B | 1-methyl-3-((3S,5R)-5- | 400.2 | Compound 33B*: Chiral RT(min): 14.35 |
| * | methyl-1-(1H-tetrazol-5- | | |
| | yl)azepan-3-yl)-1-(6- | | |
| | (trifluoromethyl)pyridazin- | | |
| | 3-yl)urea | | |
| 33C | 1-methyl-3-((3R,5S)-5- | 400.2 | Compound 33C*: Chiral RT(min): 15.99 |
| * | methyl-1-(1H-tetrazol-5- | | |
| | yl)azepan-3-yl)-1-(6- | | |
| | (trifluoromethyl)pyridazin- | | |
| | 3-yl)urea | | |
| 33D | 1-methyl-3-((3S,5S)-5- | 400.2 | Compound 33D*: Chiral RT(min): 15.52 |
| * | methyl-1-(1H-tetrazol-5- | | |
| | yl)azepan-3-yl)-1-(6- | | |
| | (trifluoromethyl)pyridazin- | | |
| | 3-yl)urea | | |
| Rac-34 | 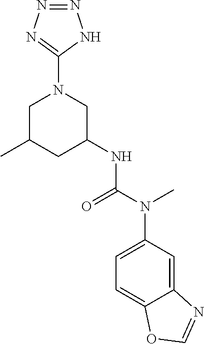 [SMILES: CC1CC(NC(=O)N(C)c2ccc3ocnc3c2)CN(c2nnn[nH]2)C1] | — | — |
| | 1-(benzo[d]oxazol-5-yl)-1- | | |
| | methyl-3-(5-methyl-1-(1H- | | |
| | tetrazol-5-yl)piperidin-3- | | |
| | yl)urea | | |
| 34A | 1-(benzo[d]oxazol-5-yl)-1- | 357.2 | Compound 34A was prepared following Protocol B and |
| | methyl-3-((3R,5S)-5- | | Example 3 using N-methylbenzo[d]oxazol-5-amine as the |
| | methyl-1-(1H-tetrazol-5- | | aniline (iv) reagent and (3R,5S)-5-methyl-1-(1H-tetrazol-5- |
| | yl)piperidin-3-yl)urea | | yl)piperidin-3-amine as the amine (i) reagent. |
| 34B | 1-(benzo[d]oxazol-5-yl)-1- | — | Compound 34B may be prepared following Protocol B and |
| | methyl-3-((3S,5S)-5- | | Example 3 using N-methylbenzo[d]oxazol-5-amine as the |
| | methyl-1-(1H-tetrazol-5- | | aniline (iv) reagent and (3S,5S)-5-methyl-1-(1H-tetrazol-5- |
| | yl)piperidin-3-yl)urea | | yl)piperidin-3-amine as the amine (i) reagent. |
| 340 | 1-(benzo[d]oxazol-5-yl)-1- | — | Compound 34C may be prepared following Protocol B and |
| | methyl-3-((3R,5R)-5- | | Example 3 using N-methylbenzo[d]oxazol-5-amine as the |
| | methyl-1-(1H-tetrazol-5- | | aniline (iv) reagent and (3R,5R)-5-methyl-1-(1H-tetrazol-5- |
| | yl)piperidin-3-yl)urea | | yl)piperidin-3-amine as the amine (i) reagent. |
| 34D | 1-(benzo[d]oxazol-5-yl)-1- | — | Compound 34D may be prepared following Protocol B and |
| | methyl-3-((3S,5R)-5- | | Example 3 using N-methylbenzo[d]oxazol-5-amine as the |
| | methyl-1-(1H-tetrazol-5- | | aniline (iv) reagent and (3S,5R)-5-methyl-1-(1H-tetrazol-5- |
| | yl)piperidin-3-yl)urea | | yl)piperidin-3-amine as the amine (i) reagent. |
| Rac-35 | 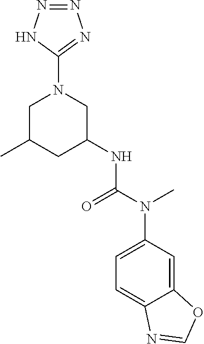 [SMILES: CC1CC(NC(=O)N(C)c2ccc3ncoc3c2)CN(c2nnn[nH]2)C1] | — | — |
| | 1-(benzo[d]oxazol-6-yl)-1- | | |
| | methyl-3-(5-methyl-1-(1H- | | |
| | tetrazol-5-yl)piperidin-3- | | |
| | yl)urea | | |
| 35A | 1-(benzo[d]oxazol-6-yl)-1- | 357.2 | Compound 35A was prepared following Protocol B and |
| | methyl-3-((3R,5S)-5- | | Example 3 using N-methylbenzo[d]oxazol-6-amine as the |
| | methyl-1-(1H-tetrazol-5- | | aniline (iv) reagent and (3R,5S)-5-methyl-1-(1H-tetrazol-5- |
| | yl)piperidin-3-yl)urea | | yl)piperidin-3-amine as the amine (i) reagent. |
| 35B | 1-(benzo[d]oxazol-6-yl)-1- | — | Compound 35B may be prepared following Protocol B and |
| | methyl-3-((3S,5S)-5- | | Example 3 using N-methylbenzo[d]oxazol-6-amine as the |
| | methyl-1-(1H-tetrazol-5- | | aniline (iv) reagent and (3S,5S)-5-methyl-1-(1H-tetrazol-5- |
| | yl)piperidin-3-yl)urea | | yl)piperidin-3-amine as the amine (i) reagent. |
| 35C | 1-(benzo[d]oxazol-6-yl)-1- | — | Compound 35C may be prepared following Protocol B and |
| | methyl-3-((3R,5R)-5- | | Example 3 using N-methylbenzo[d]oxazol-6-amine as the |
| | methyl-1-(1H-tetrazol-5- | | aniline (iv) reagent and (3R,5R)-5-methyl-1-(1H-tetrazol-5- |
| | yl)piperidin-3-yl)urea | | yl)piperidin-3-amine as the amine (i) reagent. |
| 35D | 1-(benzo[d]oxazol-6-yl)-1- | — | Compound 35D may be prepared following Protocol B and |
| | methyl-3-((3S,5R)-5- | | Example 3 using N-methylbenzo[d]oxazol-6-amine as the |
| | methyl-1-(1H-tetrazol-5- | | aniline (iv) reagent and (3S,5R)-5-methyl-1-(1H-tetrazol-5- |
| | yl)piperidin-3-yl)urea | | yl)piperidin-3-amine as the amine (i) reagent. |
| Rac-36 | 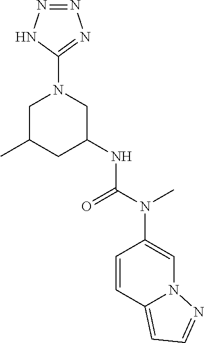 [SMILES: CC1CC(NC(=O)N(C)c2ccc3ccnn3c2)CN(c2nnn[nH]2)C1] | — | — |
| | 1-methyl-3-(5-methyl-1- | | |
| | (1H-tetrazo1-5-yl)piperidin- | | |
| | 3-yl)-1-(pyrazolo[1,5- | | |
| | alpyridin-6-yl)urea | | |
| 36A | 1-methyl-3-((3R,5S)-5- | 356.2 | Compound 36A was prepared following Protocol B and |
| | methyl-1-(1H-tetrazol-5- | | Example 3 using N-methylpyrazolo[1,5-a]pyridin-6-amine as |
| | yl)piperidin-3-yl)-1- | | the aniline (iv) reagent and (3R,5S)-5-methyl-1-(1H-tetrazol- |
| | (pyrazolo[1,5-a]pyridin-6- | | 5-yl)piperidin-3-amine as the amine (i) reagent. |
| | yl)urea | | |
| 36B | 1-methyl-3-((3S,5S)-5- | — | Compound 36B may be prepared following Protocol B and |
| | methyl-1-(1H-tetrazol-5- | | Example 3 using N-methylpyrazolo[1,5-a]pyridin-6-amine as |
| | yl)piperidin-3-yl)-1- | | the aniline (iv) reagent and (3S,5S)-5-methyl-1-(1H-tetrazol-5- |
| | (pyrazolo[1,5-a]pyridin-6- | | yl)piperidin-3-amine as the amine (i) reagent. |
| | yl)urea | | |
| 36C | 1-methyl-3-((3R,5R)-5- | — | Compound 36C may be prepared following Protocol B and |
| | methyl-1-(1H-tetrazol-5- | | Example 3 using N-methylpyrazolo[1,5-a]pyridin-6-amine as |
| | yl)piperidin-3-yl)-1- | | the aniline (iv) reagent and (3R,5R)-5-methyl-1-(1H-tetrazol- |
| | (pyrazolo[1,5-a]pyridin-6- | | 5-yl)piperidin-3-amine as the amine (i) reagent. |
| | yl)urea | | |
| 36D | 1-methyl-3-((3S,5R)-5- | — | Compound 36D may be prepared following Protocol B and |
| | methyl-1-(1H-tetrazol-5- | | Example 3 using N-methylpyrazolo[1,5-a]pyridin-6-amine as |
| | yl)piperidin-3-yl)-1- | | the aniline (iv) reagent and (3S,5R)-5-methyl-1-(1H-tetrazol- |
| | (pyrazolo[1,5-a]pyridin-6- | | 5-yl)piperidin-3-amine as the amine (i) reagent. |
| | yl)urea | | |
| Rac-37 | 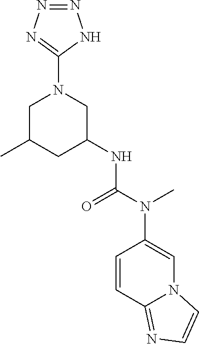 [SMILES: CC1CC(NC(=O)N(C)c2ccc3nccn3c2)CN(c2nnn[nH]2)C1] | — | — |
| | 1-(imidazo[1,2-a]pyridin-6- | | |
| | yl)-1-methyl-3-(5-methyl- | | |
| | 1-(1H-tetrazol-5- | | |
| | yl)piperidin-3-yl)urea | | |
| 37A | 1-(imidazo[1,2-a]pyridin-6- | 356.2 | Compound 37A was prepared following Protocol B and |
| | yl)-1-methyl-3-((3R,5S)-5- | | Example 3 using N-methylimidazo[1,2-a]pyridin-6-amine as |
| | methyl-1-(1H-tetrazol-5- | | the aniline (iv) reagent and (3R,5S)-5-methyl-1-(1H-tetrazol- |
| | yl)piperidin-3-yl)urea | | 5-yl)piperidin-3-amine as the amine (i) reagent. |
| 37B | 1-(imidazo[1,2-a]pyridin-6- | — | Compound 37B may be prepared following Protocol B and |
| | yl)-1-methyl-3-((3S,5S)-5- | | Example 3 using N-methylimidazo[1,2-a]pyridin-6-amine as |
| | methyl-1-(1H-tetrazol-5- | | the aniline (iv) reagent and (3S,5S)-5-methyl-1-(1H-tetrazol-5- |
| | yl)piperidin-3-yl)urea | | yl)piperidin-3-amine as the amine (i) reagent. |
| 37C | 1-(imidazo[1,2-a]pyridin-6- | — | Compound 37C may be prepared following Protocol B and |
| | yl)-1-methyl-3-((3R,5R)-5- | | Example 3 using N-methylimidazo[1,2-a]pyridin-6-amine as |
| | methyl-1-(1H-tetrazol-5- | | the aniline (iv) reagent and (3R,5R)-5-methyl-1-(1H-tetrazol- |
| | yl)piperidin-3-yl)urea | | 5-yl)piperidin-3-amine as the amine (i) reagent. |
| 37D | 1-(imidazo[1,2-a]pyridin-6- | — | Compound 37D may be prepared following Protocol B and |
| | yl)-1-methyl-3-((3S,5R)-5- | | Example 3 using N-methylimidazo[1,2-a]pyridin-6-amine as |
| | methyl-1-(1H-tetrazol-5- | | the aniline (iv) reagent and (3S,5R)-5-methyl-1-(1H-tetrazol- |
| | yl)piperidin-3-yl)urea | | 5-yl)piperidin-3-amine as the amine (i) reagent. |
| Rac-38 | 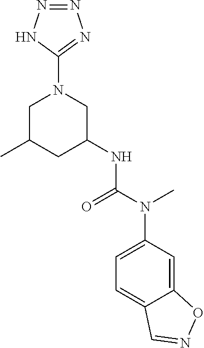 [SMILES: CC1CC(NC(=O)N(C)c2ccc3cnoc3c2)CN(c2nnn[nH]2)C1] | — | — |
| | 1-(benzo[d]isoxazol-6-yl)- | | |
| | 1-methyl-3-(5-methyl-1- | | |
| | (1H-tetrazol-5-yl)piperidin- | | |
| | 3-yl)urea | | |
| 38A | 1-(benzo[d]isoxazol-6-yl)- | 357.2 | Compound 38A was prepared following Protocol B and |
| | 1-methyl-3-((3R,5S)-5- | | Example 3 using N-methylbenzo[d]isoxazol-6-amine as the |
| | methyl-1-(1H-tetrazol-5- | | aniline (iv) reagent and (3R,5S)-5-methyl-1-(1H-tetrazol-5- |
| | yl)piperidin-3-yl)urea | | yl)piperidin-3-amine as the amine (i) reagent. |
| 38B | 1-(benzo[d]isoxazol-6-yl)- | — | Compound 38B may be prepared following Protocol B and |
| | 1-methyl-3-((3S,5S)-5- | | Example 3 using N-methylbenzo [d]isoxazol-6-amine as the |
| | methyl-1-(1H-tetrazol-5- | | aniline (iv) reagent and (3S,5S)-5-methyl-1-(1H-tetrazol-5- |
| | yl)piperidin-3-yl)urea | | yl)piperidin-3-amine as the amine (i) reagent. |
| 38C | 1-(benzo[d]isoxazol-6-yl)- | — | Compound 38C may be prepared following Protocol B and |
| | 1-methyl-3-((3R,5R)-5- | | Example 3 using N-methylbenzo[d]isoxazol-6-amine as the |
| | methyl-1-(1H-tetrazol-5- | | aniline (iv) reagent and (3R,5R)-5-methyl-1-(1H-tetrazol-5- |
| | yl)piperidin-3-yl)urea | | yl)piperidin-3-amine as the amine (i) reagent. |
| 38D | 1-(benzo[d]isoxazol-6-yl)- | — | Compound 38D may be prepared following Protocol B and |
| | 1-methyl-3-((3S,5R)-5- | | Example 3 using N-methylbenzo[d]isoxazol-6-amine as the |
| | methyl-1-(1H-tetrazol-5- | | aniline (iv) reagent and (3S,5R)-5-methyl-1-(1H-tetrazol-5- |
| | yl)piperidin-3-yl)urea | | yl)piperidin-3-amine as the amine (i) reagent. |
| Rac-39 | 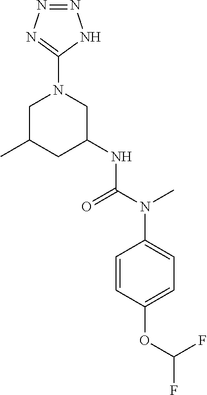 [SMILES: CC1CC(NC(=O)N(C)c2ccc(OC(F)F)cc2)CN(c2nnn[nH]2)C1] | — | — |
| | 1-(4- | | |
| | (difluoromethoxy)phenyl)- | | |
| | 1-methyl-3-(5-methyl-1- | | |
| | (1H-tetrazo1-5-yl)piperidin- | | |
| | 3-yl)urea | | |
| 39A | 1-(4- | 382.1 | Compound 39A was prepared following Protocol B and |
| | (difluoromethoxy)phenyl)- | | Example 3 using 4-(difluoromethoxy)-N-methylaniline as the |
| | 1-methyl-3-((3R,5S)-5- | | aniline (iv) reagent and (3R,5S)-5-methyl-1-(1H-tetrazol-5- |
| | methyl-1-(1H-tetrazol-5- | | yl)piperidin-3-amine as the amine (i) reagent. |
| | yl)piperidin-3-yl)urea | | |
| 39B | 1-(4- | — | Compound 39B may be prepared following Protocol B and |
| | (difluoromethoxy)phenyl)- | | Example 3 using 4-(difluoromethoxy)-N-methylaniline as the |
| | 1-methyl-3-((3S,5S)-5- | | aniline (iv) reagent and (3S,5S)-5-methyl-1-(1H-tetrazol-5- |
| | methyl-1-(1H-tetrazol-5- | | yl)piperidin-3-amine as the amine (i) reagent. |
| | yl)piperidin-3-yl)urea | | |
| 39C | 1-(4- | — | Compound 39C may be prepared following Protocol B and |
| | (difluoromethoxy)phenyl)- | | Example 3 using 4-(difluoromethoxy)-N-methylaniline as the |
| | 1-methyl-3-((3R,5R)-5- | | aniline (iv) reagent and (3R,5R)-5-methyl-1-(1H-tetrazol-5- |
| | methyl-1-(1H-tetrazol-5- | | yl)piperidin-3-amine as the amine (i) reagent. |
| | yl)piperidin-3-yl)urea | | |
| 39D | 1-(4- | — | Compound 39D may be prepared following Protocol B and |
| | (difluoromethoxy)phenyl)- | | Example 3 using 4-(difluoromethoxy)-N-methylaniline as the |
| | 1-methyl-3-((3S,5R)-5- | | aniline (iv) reagent and (3S,5R)-5-methyl-1-(1H-tetrazol-5- |
| | methyl-1-(1H-tetrazol-5- | | yl)piperidin-3-amine as the amine (i) reagent. |
| | yl)piperidin-3-yl)urea | | |
| Rac-40 | 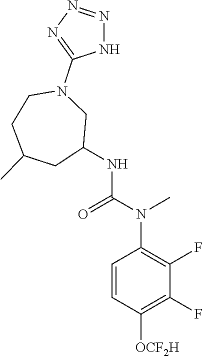 | 432.2 | Rac-40 and Compounds 40A*, 40B*, 40C* and 40D* were prepared following Protocol B and Example 2 using 4- (difluoromethoxy)-2,3-difluoro-N-methylaniline (iv) reagent and 5-methyl-1-(1H-tetrazol-5-yl)azepan-3-amine as the amine (i) reagent. Rac-40 was then subjected to prep chiral HPLC (XBridge Prep Phenyl Column, 19*250 mm, 5 μm; Mobile Phase A: Water (10mmol/L NH₄HCO₃), Mobile Phase B: acetonitrile; Flow rate: 25 mL/min; Gradient: 20% B to 30% B in 9 min; Wave Length: 254 nm/220 nm) to provide a mixture of compounds 40B* and 40C* as the first eluting peak, and a mixture of compounds 40A* and 40D* as the second eluting peak. Chiral separation conditions for assumed cis isomers, 40A* and 40D*: Column: CHIRALPAK IG 2*25 cm, 5 um; Mobile Phase A: Hexanes (0.1% trifluoroacetic acid), Mobile Phase B: ethanol:dichloromethane = 1:1; Flow rate: 20 mL/min; Gradient: 15% B to 15% B in 15.5 min; Wave Length: 220/254 nm. Chiral separation conditions for assumed trans isomers, 40B* and 40C*: Column: Lux 5 um Cellulose-2 2.12*25 cm, 5 μm; Mobile Phase A: Hexanes (0.1% trifluoroacetic acid), Mobile Phase B: isopropanol; Flow rate: 20 mL/min; Gradient: 30% B to 30% B in 17.5 min; Wave Length: 220/254 nm. |
| | 1-(4-(difluoromethoxy)-2,3- | | |
| | difluorophenyl)-1-methyl- | | |
| | 3-(5-methyl-1-(1H-tetrazol- | | |
| | 5-yl)azepan-3-yl)urea | | |
| 40A | 1-(4-(difluoromethoxy)-2,3- | 432.2 | Compound 40A*: Chiral RT(min): 9.82 |
| * | difluorophenyl)-1-methyl- | | |
| | 3-((3R,5R)-5-methyl-1- | | |
| | (1H-tetrazol-5-yl)azepan-3- | | |
| | yl)urea | | |
| 40B | 1-(4-(difluoromethoxy)-2,3- | 432.2 | Compound 40B*: Chiral RT(min): 9.41 |
| * | difluorophenyl)-1-methyl- | | |
| | 3-((3S,5R)-5-methyl-1-(1H- | | |
| | tetrazol-5-yl)azepan-3- | | |
| | yl)urea | | |
| 40C | 1-(4-(difluoromethoxy)-2,3- | 432.2 | Compound 40C*: Chiral RT(min): 15.64 |
| * | difluorophenyl)-1-methyl- | | |
| | 3-((3R,5S)-5-methyl-1-(1H- | | |
| | tetrazol-5-yl)azepan-3- | | |
| | yl)urea | | |
| 40D | 1-(4-(difluoromethoxy)-2,3- | 432.2 | Compound 40D*: Chiral RT(min): 14.12 |
| * | difluorophenyl)-1-methyl- | | |
| | 3-((3S,5S)-5-methyl-1-(1H- | | |
| | tetrazol-5-yl)azepan-3- | | |
| | yl)urea | | |
| Rac-41 | 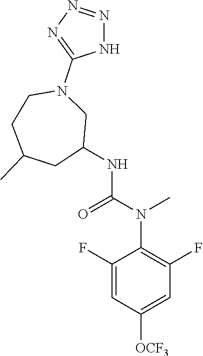 [SMILES: CC1CCN(c2nnn[nH]2)CC(NC(=O)N(C)c2c(F)cc(OC(F)(F)F)cc2F)C1] | 450.1 | Rac-41 and Compounds 41A*, 41B*, 41C* and 41D* were prepared following Protocol B and Example 2 using 2,6- difluoro-N-methyl-4-(trifluoromethoxy)aniline as the aniline (iv) reagent and 5-methyl-1-(1H-tetrazol-5-yl)azepan-3-amine as the amine (i) reagent. Rac-41 was then subjected to prep chiral HPLC (XBridge Prep Phenyl Column, 19*250 mm, 5 um; Mobile Phase A: Water (10mmol/L NH4HCO3), Mobile Phase B: acetonitrile; Flow rate: 25 mL/min; Gradient: 20% B to 30% B in 9 min; Wave Length: 254nm/220nm) to provide a mixture of compounds 41B* and 41C* as the first eluting peak, and a mixture of compounds 41A* and 41D* as the second eluting peak. Chiral separation conditions for assumed cis isomers, 41A* and 41D*: Column: CHIRALPAK IG 2*25 cm, 5 um; Mobile Phase A: Hexanes (0.1% trifluoroacetic acid), Mobile Phase B: EtOH: DCM= 1: 1; Flow rate: 20 mL/min; Gradient: 15% B to 15% B in 15.5 min; Wave Length: 220/254 nm. Chiral separation conditions for assumed trans isomers, 41B* and 41C*: Column: Lux 5 um Cellulose-2 2.12*25 cm, 5 μm; Mobile Phase A: Hexanes (0.1% trifluoroacetic acid), Mobile Phase B: IPA; Flow rate: 20 mL/min; Gradient: 30% B to 30% B in 17.5 min; Wave Length: 220/254 nm. |
| | 1-(2,6-difluoro-4- | | |
| | (trifluoromethoxy)phenyl)- | | |
| | 1-methyl-3-(5-methyl-1- | | |
| | (1H-tetrazol-5-yl)azepan-3- | | |
| | yl)urea | | |
| 41A | 1-(2,6-difluoro-4- | 450.1 | Compound 41A*: Chiral RT(min): 9.61 |
| * | (trifluoromethoxy)phenyl)- | | |
| | 1-methyl-3-((3R,5R)-5- | | |
| | methyl-1-(1H-tetrazol-5- | | |
| | yl)azepan-3-yl)urea | | |
| 41B | 1-(2,6-difluoro-4- | 450.1 | Compound 41B*: Chiral RT(min): 17.39 |
| * | (trifluoromethoxy)phenyl)- | | |
| | 1-methyl-3-((3S,5R)-5- | | |
| | methyl-1-(1H-tetrazol-5- | | |
| | yl)azepan-3-yl)urea | | |
| 41C | 1-(2,6-difluoro-4- | 450.1 | Compound 41C*: Chiral RT(min): 13.87 |
| * | (trifluoromethoxy)phenyl)- | | |
| | 1-methyl-3-((3R,5S)-5- | | |
| | methyl-1-(1H-tetrazol-5- | | |
| | yl)azepan-3-yl)urea | | |
| 41D | 1-(2,6-difluoro-4- | 450.1 | Compound 41D*: Chiral RT(min): 6.44 |
| * | (trifluoromethoxy)phenyl)- | | |
| | 1-methyl-3-((3S,5S)-5- | | |
| | methyl-1-(1H-tetrazol-5- | | |
| | yl)azepan-3-yl)urea | | |
| Rac-43 | 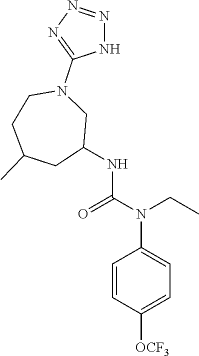 [SMILES: CCN(C(=O)NC1CC(C)CCN(c2nnn[nH]2)C1)c1ccc(OC(F)(F)F)cc1] | 428.2 | Rac-43 and Compounds 43A*, 43B*, 43C* and 43D* were prepared following Protocol B and Example 2 using N-ethyl- 4-(trifluoromethoxy)aniline as the aniline (iv) reagent and 5- methyl-1-(1H-tetrazol-5-yl)azepan-3-amine as the amine (i) reagent. Rac-43 was then subjected to prep chiral HPLC (XBridge Prep OBD C18 Column 30*150 mm; Mobile Phase A: Water(10 mmol/L NH₄HCO₃), Mobile Phase B: acetonitrile; Flow rate: 60 mL/min; Gradient: 21% B to 31% B in 7.8 min; Wave Length: 254 nm/220 nm) to provide a mixture of compounds 43B* and 43C* as the first eluting peak, and a mixture of compounds 43A* and 43D* as the second eluting peak. Chiral separation conditions for assumed cis isomers, 43A* and 43D*: Column: Lux 5 um Cellulose-4, 2.12*25 cm, 5 μm; Mobile Phase A: Hexanes (0.1% trifluoroacetic acid), Mobile Phase B: ethanol; Flow rate: 20 mL/min; Gradient: 10% B to 10% B in 29 min; Wave Length: 220/254 nm. Chiral separation conditions for assumed trans isomers, 43B* and 43C*: Column: Lux 5 um Cellulose-4, 2.12*25 cm, 5 μm; Mobile Phase A: Hexanes (0.1% trifluoroacetic acid), Mobile Phase B: ethanol; Flow rate: 20 mL/min; Gradient: 30% B to 30% B in 8 min; Wave Length: 220/254 nm. |
| | 1-ethyl-3-(5-methyl-1-(1H- | | |
| | tetrazol-5-yl)azepan-3-yl)- | | |
| | 1-(4- | | |
| | (trifluoromethoxy)phenyl) | | |
| | urea | | |
| 43A | 1-ethyl-3-((3R,5R)-5- | 428.2 | Compound 43A*: Chiral RT(min): 7.45 |
| * | methyl-1-(1H-tetrazol-5- | | |
| | yl)azepan-3-yl)-1-(4- | | |
| | (trifluoromethoxy)phenyl) | | |
| | urea | | |
| 43B | 1-ethyl-3-((3S,5R)-5- | 428.2 | Compound 43B*: Chiral RT(min): 23.17 |
| * | methyl-1-(1H-tetrazol-5- | | |
| | yl)azepan-3-yl)-1-(4- | | |
| | (trifluoromethoxy)phenyl) | | |
| | urea | | |
| 43C | 1-ethyl-3-((3R,5S)-5- | 428.2 | Compound 43C*: Chiral RT(min): 26.13 |
| * | methyl-1-(1H-tetrazol-5- | | |
| | yl)azepan-3-yl)-1-(4- | | |
| | (trifluoromethoxy)phenyl) | | |
| | urea | | |
| 43D | 1-ethyl-3-((3S,5S)-5- | 428.2 | Compound 43D*: Chiral RT(min): 6.05 |
| * | methyl-1-(1H-tetrazol-5- | | |
| | yl)azepan-3-yl)-1-(4- | | |
| | (trifluoromethoxy)phenyl) | | |
| | urea | | |
| Rac-44 | 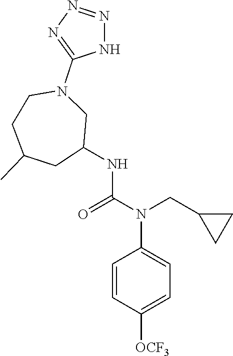 [SMILES: CC1CCN(c2nnn[nH]2)CC(NC(=O)N(CC2CC2)c2ccc(OC(F)(F)F)cc2)C1] | 454.2 | Rac-44 and Compounds 44A*, 44B*, 44C* and 44D* were prepared following Protocol B and Example 2 using N- (cyclopropylmethyl)-4-(trifluoromethoxy)aniline as the aniline (iv) reagent and 5-methyl-1-(1H-tetrazol-5-yl)azepan-3-amine as the amine (i) reagent. Rac-44 was then subjected to prep chiral HPLC (XSelect CSH OBD Column 19*250 mm, 5 umn; Mobile Phase A: Water (0.1% formic acid), Mobile Phase B: MeOH; Flow rate: 20 mL/min; Gradient: isocratic 72-77; Wave Length: 254 nm/220 nm) to provide a mixture of compounds 44B* and 44C* as the first eluting peak, and a mixture of compounds 44A* and 44D* as the second eluting peak. Chiral separation conditions for assumed cis isomers, 44A* and 44D*: Column: CHIRALPAK ID, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 15% B to 15% B in 27 min; Wave Length: 220/254 nm. Chiral separation conditions for assumed trans isomers, 44B* and 44C*: Column: CHIRALPAK ID, 2*25 cm, 5 μm; Mobile Phase A: Hexanes (0.1% trifluoroacetic acid), Mobile Phase B: Ethanol:dichloromethane = 1: 1; Flow rate: 20 mL/min; Gradient: 10% B to 10% B in 29 min; Wave Length: 220/254 nm. |
| | 1-(cyclopropylmethyl)-3- | | |
| | (5-methyl-1-(1H-tetrazol-5- | | |
| | yl)azepan-3-yl)-1-(4- | | |
| | (trifluoromethoxy)phenyl) | | |
| | urea | | |
| 44A | 1-(cyclopropylmethyl)-3- | 454.2 | Compound 44A*: Chiral RT(min): 21.93 |
| * | ((3R,5R)-5-methyl-1-(1H- | | |
| | tetrazol-5-yl)azepan-3-yl)- | | |
| | 1-(4- | | |
| | (trifluoromethoxy)phenyl) | | |
| | urea | | |
| 44B | 1-(cyclopropylmethyl)-3- | 454.2 | Compound 44B*: Chiral RT(min): 20.03 |
| * | ((3S,5R)-5-methyl-1-(1H- | | |
| | tetrazol-5-yl)azepan-3-yl)- | | |
| | 1-(4- | | |
| | (trifluoromethoxy)phenyl) | | |
| | urea | | |
| 44C | 1-(cyclopropylmethyl)-3- | 454.2 | Compound 44C*: Chiral RT(min): 23.21 |
| * | ((3R,5S)-5-methyl-1-(1H- | | |
| | tetrazol-5-yl)azepan-3-yl)- | | |
| | 1-(4- | | |
| | (trifluoromethoxy)phenyl) | | |
| | urea | | |
| 44D | 1-(cyclopropylmethyl)-3- | 454.2 | Compound 44D*: Chiral RT(min): 18.76 |
| * | ((3S,5S)-5-methyl-1-(1H- | | |
| | tetrazol-5-yl)azepan-3-yl)- | | |
| | 1-(4- | | |
| | (trifluoromethoxy)phenyl) | | |
| | urea | | |
| Rac-45 | 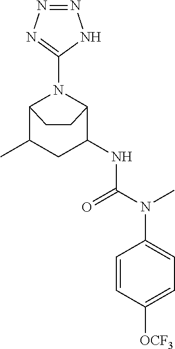 [SMILES: CC1CC(NC(=O)N(C)c2ccc(OC(F)(F)F)cc2)C2CCC1N2c1nnn[nH]1] | 426.1 | Rac-45, 45′*, 45″*, 45A*, 45B*, 45C* and 45D* were prepared following Protocol B and Example 4 using N- methyl-4-(trifluoromethoxy)aniline as the aniline (iv) reagent and 4-methyl-8-(1H-tetrazol-5-yl)-8-azabicyclo[3.2.1]octan-2- amine as the amine (i) reagent. Rac-45 was then subjected to prep-HPLC using the following conditions: XBridge Prep OBD C18 Column, 30*150 mm, 5 um; Mobile Phase A: Water (10 mmol/L NH₄HCO₃), Mobile Phase B: acetonitrile; Flow rate: 60 mL/min; Gradient: 21% B to 31% B in 10 min; Wave Length: 254 nm/220 nm to afford a mixture of compounds 45B* and 45C* as the first eluting peak, and a mixture of compounds 45A* and 45D* as the second eluting peak. |
| | 1-methyl-3-(4-methyl-8- | | |
| | (1H-tetrazol-5-yl)-8- | | |
| | azabicyclo [3.2.1]octan-2- | | |
| | yl)-1-(4- | | |
| | (trifluoromethoxy)phenyl) | | Chiral separation conditions for assumed trans isomers, 45B* |
| | urea | | and 45C*: Column: CHIRALPAK ID, 2*25 cm, 5 μm; Mobile |
| 45′* | 1-methyl-3-((1R,4R,5S)-4- | 426.1 | Phase A: Hexanes (0.1% trifluoroacetic acid), Mobile Phase |
| | methyl-8-(1H-tetrazol-5- | | B: ethanol: dichloromethane = 1:1; Flow rate: 20 mL/min; |
| | yl)-8- | | Gradient: 30% B to 30% B in 6.5 min; Wave Length: 220/254 |
| | azabicyclo[3.2.1]octan-2- | | nm. |
| | yl)-1-(4- | | |
| | (trifluoromethoxy)phenyl) | | Chiral separation conditions for assumed cis isomers, 45A* |
| | urea | | and 45D*: CHIRAL ART Cellulose-SC, 2*25 cm, 5 μm; |
| 45″ | 1-methyl-3-((1S,4S,5R)-4- | | Mobile Phase A: Hexanes (0.1% trifluoroacetic acid), Mobile |
| * | methyl-8-(1H-tetrazol-5- | | Phase B: ethanol: dichloromethane = 1:1; Flow rate: 20 |
| | yl)-8- | | mL/min; Gradient: 30% B to 30% B in 9 min; Wave Length: |
| | azabicyclo[3.2.1]octan-2- | 426.1 | 220/254 nm. |
| | yl)-1-(4- | | |
| | (trifluoromethoxy)phenyl) | | |
| | urea | | |
| 45A | 1-methyl-3- | 426.1 | Compound 45A*: Chiral RT(min): 6.28 |
| * | ((1R,2R,4R,5S)-4-methyl- | | |
| | 8-(1H-tetrazol-5-yl)-8- | | |
| | azabicyclo[3.2.1]octan-2- | | |
| | yl)-1-(4- | | |
| | (trifluoromethoxy)phenyl) | | |
| | urea | | |
| 45B | 1-methyl-3-((1R,2S,4R,5S)- | 426.1 | Compound 45B*: Chiral RT(min): 5.43 |
| * | 4-methyl-8-(1H-tetrazol-5- | | |
| | yl)-8- | | |
| | azabicyclo[3.2.1]octan-2- | | |
| | yl)-1-(4- | | |
| | (trifluoromethoxy)phenyl) | | |
| | urea | | |
| 45C | 1-methyl-3-((1S,2R,4S,5R)- | 426.1 | Compound 45C*: Chiral RT(min): 4.33 |
| * | 4-methyl-8-(1H-tetrazol-5- | | |
| | yl)-8- | | |
| | azabicyclo[3.2.1]octan-2- | | |
| | yl)-1-(4- | | |
| | (trifluoromethoxy)phenyl) | | |
| | urea | | |
| 45D | 1-methyl-3-((1S,2S,4S,5R)- | 426.1 | Compound 45D*: Chiral RT(min): 7.57 |
| * | 4-methyl-8-(1H-tetrazol-5- | | |
| | yl)-8- | | |
| | azabicyclo[3.2.1]octan-2- | | |
| | yl)-1-(4- | | |
| | (trifluoromethoxy)phenyl) | | |
| | urea | | |
| Rac-46 | 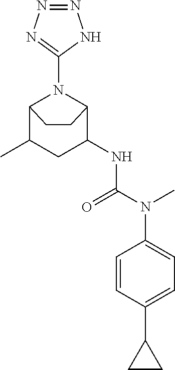 | 382.2 | Rac-46, 46′*, 46″*, 46A*, 46B*, 46C* and 46D* were prepared following Protocol B and Example 4 using N- methyl-4-(cyclopropyl)aniline as the aniline (iv) reagent and 4-methyl-8-(1H-tetrazol-5-yl)-8-azabicyclo[3.2.1]octan-2- amine as the amine (i) reagent. Rac-46 was then subjected to prep-HPLC using the following conditions: XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 18% B to 34% B in 20 min; Wave Length: 254 nm/220 nm to afford a mixture of compounds 46B* and 46C* as the first eluting peak, and a mixture of compounds 46A* and 46D* as the second eluting peak. |
| | 1-(4-cyclopropylphenyl)-1- | | |
| | methyl-3-(4-methyl-8-(1H- | | |
| | tetrazol-5-yl)-8- | | Chiral separation conditions for assumed trans isomers, 46B* |
| | azabicyclo[3.2.1]octan-2- | | and 46C*: CHIRALPAK IG, 2*25 cm, 5 μm; Mobile Phase A: |
| | yl)urea | | Hexanes (0.1% trifluoroacetic acid), Mobile Phase B: |
| 46′* | 1-(4-cyclopropylphenyl)-1- | 382.2 | isopropanol: dichloromethane = 1:1; Flow rate: 20 mL/min; |
| | methyl-3-((1R,4R,5S)-4- | | Gradient: 30% B to 30% B in 10 min; Wave Length: 220/254 |
| | methyl-8-(1H-tetrazol-5- | | nm. |
| | yl)-8- | | |
| | azabicyclo[3.2.1]octan-2- | | Chiral separation conditions for assumed cis isomers, 46A* |
| | yl)urea | | and 46D*: CHIRAL ART Cellulose-SC, 2*25 cm, 5 μm; |
| 46″ | 1-(4-cyclopropylphenyl)-1- | 382.2 | Mobile Phase A: Hexanes (0.1% trifluoroacetic acid), Mobile |
| * | methyl-3-((1S,4S,5R)-4- | | Phase B: Ethanol:dichloromethane = 1:1; Flow rate: 20 |
| | methyl-8-(1H-tetrazol-5- | | mL/min; Gradient: 35% B to 35% B in 9 min; Wave Length: |
| | yl)-8- | | 220/254 nm. |
| | azabicyclo[3.2.1]octan-2- | | |
| | yl)urea | | |
| 46A | 1-(4-cyclopropylphenyl)-1- | 382.2 | Compound 46A*: Chiral RT(min): 6.67 |
| * | methyl-3-((1R,2R,4R,5S)- | | |
| | 4-methyl-8-(1H-tetrazol-5- | | |
| | yl)-8- | | |
| | azabicyclo[3.2.1]octan-2- | | |
| | yl)urea | | |
| 46B | 1-(4-cyclopropylphenyl)-1- | 382.2 | Compound 46B*: Chiral RT(min): 7.66 |
| * | methyl-3-((1R,2S,4R,5S)-4- | | |
| | methyl-8-(1H-tetrazol-5- | | |
| | yl)-8- | | |
| | azabicyclo [3.2.1]octan-2- | | |
| | yl)urea | | |
| 46C | 1-(4-cyclopropylphenyl)-1- | 382.2 | Compound 46C*: Chiral RT(min): 5.70 |
| * | methyl-3-((1S,2R,4S,5R)-4- | | |
| | methyl-8-(1H-tetrazol-5- | | |
| | yl)-8- | | |
| | azabicyclo [3.2.1]octan-2- | | |
| | yl)urea | | |
| 46D | 1-(4-cyclopropylphenyl)-1- | 382.2 | Compound 46D*: Chiral RT(min): 8.16 |
| * | methyl-3-((1S,2S,4S,5R)-4- | | |
| | methyl-8-(1H-tetrazol-5- | | |
| | yl)-8- | | |
| | azabicyclo[3.2.1]octan-2- | | |
| | yl)urea | | |
| Rac-47 | 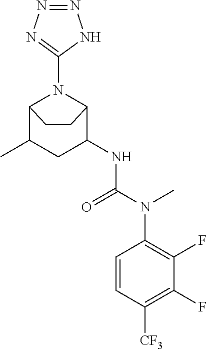 [SMILES: CC1CC(NC(=O)N(C)c2ccc(C(F)(F)F)c(F)c2F)C2CCC1N2c1nnn[nH]1] | 446.1 | Rac-47, 47′*, 47″*, 47A*, 47B*, 47C* and 47D* were prepared following Protocol B and Example 4 using 2,3- difluoro-N-methyl-4-(trifluoromethyl)aniline as the aniline (iv) reagent and 4-methyl-8-(1H-tetrazol-5-yl)-8- azabicyclo[3.2.1]octan-2-amine as the amine (i) reagent. Rac-47 was then subjected to prep-HPLC using the following conditions: Column: XBridge Prep Phenyl OBD Column 19*250 mm, 5 μm; Mobile Phase A: Water (0.05% trifluoroacetic acid), Mobile Phase B: acetonitrile; Flow rate: 25 mL/min; Gradient: 36% B to 46% B in 10 min; Wave Length: 254/220 nm to afford a mixture of compounds 47B* and 47C* as the first eluting peak, and a mixture of compounds 47A* and 47D* as the second eluting peak. |
| | 1-(2,3-difluoro-4- | | |
| | (trifluoromethyl)phenyl)-1- | | |
| | methyl-3-(4-methyl-8-(1H- | | |
| | tetrazol-5-yl)-8- | | |
| | azabicyclo[3.2.1]octan-2- | | Chiral separation conditions for assumed trans isomers, 47B* |
| | yl)urea | | and 47C*: Column: CHIRALPAK IF, 2*25 cm, 5 μm; Mobile |
| 47′* | 1-(2,3-difluoro-4- | 446.1 | Phase A: Hexanes (0.1% trifluoroacetic acid), Mobile Phase |
| | (trifluoromethyl)phenyl)-1- | | B: ethanol: dichloromethane = 1: 1; Flow rate: 20 mL/min; |
| | methyl-3-((1R,2R,5S)-4- | | Gradient: 25% B to 25% B in 8 min; Wave Length: 220/254 |
| | methyl-8-(1H-tetrazol-5- | | nm. |
| | yl)-8- | | |
| | azabicyclo[3.2.1]octan-2- | | Chiral separation conditions for assumed cis isomers, 47A* |
| | yl)urea | | and 47D*: Column: CHIRALPAK SC 2*25 cm, 5 μm; Mobile |
| 47″ | 1-(2,3-difluoro-4- | 446.1 | Phase A: Hexanes (0.1% trifluoroacetic acid), Mobile Phase |
| * | (trifluoromethyl)phenyl)-1- | | B: ethanol: dichloromethane = 1:1; Flow rate: 20 mL/min; |
| | methyl-3-((1S,2S,5R)-4- | | Gradient: isocratic 15; Wave Length: 220/254 nm. |
| | methyl-8-(1H-tetrazol-5- | | |
| | yl)-8- | | |
| | azabicyclo [3.2.1]octan-2- | | |
| | yl)urea | | |
| 47A | 1-(2,3-difluoro-4- | 446.1 | Compound 47A*: Chiral RT(min): 17.83 |
| * | (trifluoromethyl)phenyl)-1- | | |
| | methyl-3-((1R,2R,4R,5S)- | | |
| | 4-methyl-8-(1H-tetrazol-5- | | |
| | yl)-8- | | |
| | azabicyclo[3.2.1]octan-2- | | |
| | yl)urea | | |
| 47B | 1-(2,3-difluoro-4- | 446.1 | Compound 47B*: Chiral RT(min): 4.98 |
| * | (trifluoromethyl)phenyl)-1- | | |
| | methyl-3-((1R,2S,4R,5S)-4- | | |
| | methyl-8-(1H-tetrazol-5- | | |
| | yl)-8- | | |
| | azabicyclo [3.2.1]octan-2- | | |
| | yl)urea | | |
| 47C | 1-(2,3-difluoro-4- | 446.1 | Compound 47C*: Chiral RT(min): 16.64 |
| * | (trifluoromethyl)phenyl)-1- | | |
| | methyl-3-((1S,2R,4S,5R)-4- | | |
| | methyl-8-(1H-tetrazol-5- | | |
| | yl)-8- | | |
| | azabicyclo [3.2.1]octan-2- | | |
| | yl)urea | | |
| 47D | 1-(2,3-difluoro-4- | 446.1 | Compound 47D*: Chiral RT(min): 6.72 |
| * | (trifluoromethyl)phenyl)-1- | | |
| | methyl-3-((1S,2S,4S,5R)-4- | | |
| | methyl-8-(1H-tetrazol-5- | | |
| | yl)-8- | | |
| | azabicyclo[3.2.1]octan-2- | | |
| | yl)urea | | |
| Rac-48 | 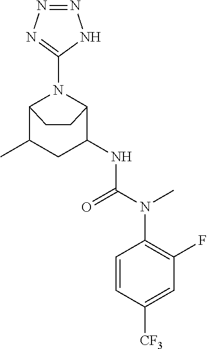 [SMILES: CC1CC(NC(=O)N(C)c2ccc(C(F)(F)F)cc2F)C2CCC1N2c1nnn[nH]1] | 455.1 | Rac-48, 48′*, 48″*, 48A*, 48B*, 48C* and 48D* were prepared following Protocol B and Example 4 using 2-fluoro- N-methyl-4-(trifluoromethyl)benzenamine as the aniline (iv) reagent and 4-methyl-8-(1H-tetrazol-5-yl)-8- azabicyclo[3.2.1]octan-2-amine as the amine (i) reagent. Rac-48 was then subjected to prep-HPLC using the following conditions: Column: XBridge Prep OBD C18 Column, 30*150 mm, 5 um; Mobile Phase A: Water (10mmol/L NH4HCO3), Mobile Phase B: acetonitrile; Flow rate: 60 mL/min; Gradient: 19% B to 29% B in 10 min, 29% B to 36% B in 15 min; Wave Length: 254 nm/220 nm to afford a mixture of compounds 48B* and 48C* as the first eluting peak, and a mixture of compounds 48A* and 48D* as the second eluting peak. Chiral separation conditions for assumed trans isomers, 48B* and 48C*: Column: CHIRALPAK IF, 2*25 cm, 5 μm; Mobile Phase A: Hexanes (0.1% trifluoroacetic acid), Mobile Phase B: ethanol: dichloromethane = 1:1; Flow rate: 20 mL/min; Gradient: 25% B to 25% B in 11 min; Wave Length: 220/254 nm. |
| | 1-(2-fluoro-4- | | |
| | (trifluoromethyl)phenyl)-1- | | |
| | methyl-3-(4-methyl-8-(1H- | | |
| | tetrazol-5-yl)-8- | | |
| | azabicyclo [3.2.1]octan-2- | | |
| | yl)urea | | |
| 48′* | 1-(2-fluoro-4- | 455.1 | |
| | (trifluoromethyl)phenyl)-1- | | |
| | methyl-3-((1R,2R,5S)-4- | | Chiral separation conditions for assumed cis isomers, 48A* |
| | methyl-8-(1H-tetrazol-5- | | and 48D*: Column: CHIRAL ART Cellulose-SC, 2*25 cm, 5 |
| | yl)-8- | | μm; Mobile Phase A: Hexanes (0.1% trifluoroacetic acid), |
| | azabicyclo[3.2.1]octan-2- | | Mobile Phase B: ethanol: dichloromethane = 1:1; Flow rate: 20 |
| | yl)urea | | mL/min; Gradient: 20% B to 20% B in 13 min; Wave Length: |
| 48″ | 1-(2-fluoro-4- | 455.1 | 220/254 nm. |
| * | (trifluoromethyl)phenyl)-1- | | |
| | methyl-3-((1S,2S,5R)-4- | | |
| | methyl-8-(1H-tetrazol-5- | | |
| | yl)-8- | | |
| | azabicyclo[3.2.1]octan-2- | | |
| | yl)urea | | |
| 48A | 1-(2-fluoro-4- | 455.1 | Compound 48A*: Chiral RT (min): 10.22 |
| * | (trifluoromethyl)phenyl)-1- | | |
| | methyl-3-((1R,2R,4R,5S)- | | |
| | 4-methyl-8-(1H-tetrazol-5- | | |
| | yl)-8- | | |
| | azabicyclo[3.2.1]octan-2- | | |
| | yl)urea | | |
| 48B | 1-(2-fluoro-4- | 455.1 | Compound 48B*: Chiral RT (min): 7.26 |
| | (trifluoromethyl)phenyl)-1- | | |
| | methyl-3-((1R,2S,4R,5S)-4- | | |
| | methyl-8-(1H-tetrazol-5- | | |
| | yl)-8- | | |
| | azabicyclo[3.2.1]octan-2- | | |
| | yl)urea | | |
| 48C | 1-(2-fluoro-4- | 455.1 | Compound 48C*: Chiral RT (min): 5.24 |
| * | (trifluoromethyl)phenyl)-1- | | |
| | methyl-3-((1S,2R,4S,5R)-4- | | |
| | methyl-8-(1H-tetrazol-5- | | |
| | yl)-8- | | |
| | azabicyclo[3.2.1]octan-2- | | |
| | yl)urea | | |
| 48D | 1-(2-fluoro-4- | 455.1 | Compound 48D*: Chiral RT (min): 12.47 |
| * | (trifluoromethyl)phenyl)-1- | | |
| | methyl-3-((1S,2S,4S,5R)-4- | | |
| | methyl-8-(1H-tetrazol-5- | | |
| | yl)-8- | | |
| | azabicyclo[3.2.1]octan-2- | | |
| | yl)urea | | |
| Rac-49 | 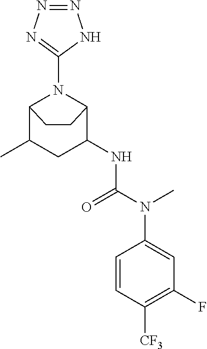 [SMILES: CC1CC(NC(=O)N(C)c2ccc(C(F)(F)F)c(F)c2)C2CCC1N2c1nnn[nH]1] | 427.2 | Rac-49, 49′*, 49″*, 49A*, 49B*, 49C* and 49D* were prepared following Protocol B and Example 4 using 3-fluoro- N-methyl-4-(trifluoromethyl)benzenamine as the aniline (iv) reagent and 4-methyl-8-(1H-tetrazol-5-yl)-8- azabicyclo[3.2.1]octan-2-amine as the amine (i) reagent. Rac-49 was then subjected to prep-HPLC using the following conditions: Column: XSelect CSH Prep C18 OBD Column, 19*250 mm, 5 um; Mobile Phase A: Water (50 mmol/L NH₄HCO₃), Mobile Phase B: acetonitrile;Flow rate: 20 mL/min; Gradient: 22% B to 32% B in 10 min; Wave Length: 254 nm/220 nm to afford a mixture of compounds 49B* and 49C* as the first eluting peak, and a mixture of compounds 49A* and 49D* as the second eluting peak. |
| | 1-(3-fluoro-4- | | |
| | (trifluoromethyl)phenyl)-1- | | |
| | methyl-3-(4-methyl-8-(1H- | | |
| | tetrazol-5-yl)-8- | | |
| | azabicyclo[3.2.1]octan-2- | | Chiral separation conditions for assumed trans isomers, 49B* |
| | yl)urea | | and 49C*: Column: CHIRALPAK IF, 2*25 cm, 5 μm; Mobile |
| 49′* | 1-(3-fluoro-4- | 427.2 | Phase A: Hexanes (0.1% trifluoroacetic acid), Mobile Phase |
| | (trifluoromethyl)phenyl)-1- | | B: ethanol: dichloromethane = 1:1; Flow rate: 20 mL/min; |
| | methyl-3-((1R,2R,5S)-4- | | Gradient: 25% B to 25% B in 12 min; Wave Length: 220/254 |
| | methyl-8-(1H-tetrazol-5- | | nm. |
| | yl)-8- | | |
| | azabicyclo[3.2.1]octan-2- | | Chiral separation conditions for assumed cis isomers, 49A* |
| | yl)urea | | and 49D*: Column: CHIRAL ART Cellulose-SC, 2*25 cm, 5 |
| 49″ | 1-(3-fluoro-4- | 427.2 | μm; Mobile Phase A: Hexanes (0.1% trifluoroacetic acid), |
| * | (trifluoromethyl)phenyl)-1- | | Mobile Phase B: ethanol: dichloromethane = 1:1; Flow rate: 20 |
| | methyl-3-((1S,2S,5R)-4- | | mL/min; Gradient: 25% B to 25% B in 10.5 min; Wave |
| | methyl-8-(1H-tetrazol-5- | | Length: 220/254 nm. |
| | yl)-8- | | |
| | azabicyclo[3.2.1]octan-2- | | |
| | yl)urea | | |
| 49A | 1-(3-fluoro-4- | 427.2 | Compound 49A*: Chiral RT (min): 7.53 |
| * | (trifluoromethyl)phenyl)-1- | | |
| | methyl-3-((1R,2R,4R,5S)- | | |
| | 4-methyl-8-(1H-tetrazol-5- | | |
| | yl)-8- | | |
| | azabicyclo[3.2.1]octan-2- | | |
| | yl)urea | | |
| 49B | 1-(3-fluoro-4- | 427.2 | Compound 49B*: Chiral RT (min): 5.69 |
| * | (trifluoromethyl)phenyl)-1- | | |
| | methyl-3-((1R,2S,4R,5S)-4- | | |
| | methyl-8-(1H-tetrazol-5- | | |
| | yl)-8- | | |
| | azabicyclo[3.2.1]octan-2- | | |
| | yl)urea | | |
| 49C | 1-(3-fluoro-4- | 427.2 | Compound 49C*: Chiral RT (min): 7.81 |
| * | (trifluoromethyl)phenyl)-1- | | |
| | methyl-3-((1S,2R,4S,5R)-4- | | |
| | methyl-8-(1H-tetrazol-5- | | |
| | yl)-8- | | |
| | azabicyclo[3.2.1]octan-2- | | |
| | yl)urea | | |
| 49D | 1-(3-fluoro-4- | 427.2 | Compound 49D*: Chiral RT (min): 8.90 |
| * | (trifluoromethyl)phenyl)-1- | | |
| | methyl-3-((1S,2S,4S,5R)-4- | | |
| | methyl-8-(1H-tetrazol-5- | | |
| | yl)-8- | | |
| | azabicyclo[3.2.1]octan-2- | | |
| | yl)urea | | |
| Rac-50 | 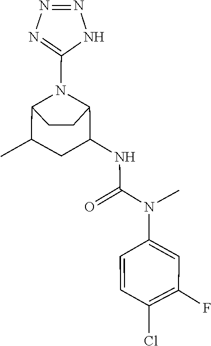 [SMILES: CC1CC(NC(=O)N(C)c2ccc(Cl)c(F)c2)C2CCC1N2c1nnn[nH]1] | 394.1 | Rac-50, 50′*, 50″*, 50A*, 50B*, 50C* and 50D* were prepared following Protocol B and Example 4 using 4-chloro- 3-fluoro-N-methylbenzenamine as the aniline (iv) reagent and 4-methyl-8-(1H-tetrazol-5-yl)-8-azabicyclo[3.2.1]octan-2- amine as the amine (i) reagent. Rac-50 was then subjected to prep-HPLC using the following conditions: Column: XBridge Prep OBD C18 Column, 30*150 mm, 5 μm; Mobile Phase A: Water (10 mmol/L NH4HCO3), Mobile Phase B: acetonitrile; Flow rate: 60 mL/min; Gradient: 15% B to 25% B in 7.8 min; Wave Length: 254 nm/220 nm to afford a mixture of compounds 50B* and 50C* as the first eluting peak, and a mixture of compounds 50A* and 50D* as the second eluting peak. |
| | 1-(4-chloro-3- | | |
| | fluorophenyl)-1-methyl-3- | | |
| | (4-methyl-8-(1H-tetrazol-5- | | |
| | yl)-8- | | Chiral separation conditions for assumed trans isomers, 50B* |
| | azabicyclo[3.2.1]octan-2- | | and 50C*: Column: CHIRAL ART Cellulose-SC, 2*25 cm, 5 |
| | yl)urea | | μm; Mobile Phase A: Hexanes (0.1% trifluoroacetic acid), |
| 50′* | 1-(4-chloro-3- | 394.1 | Mobile Phase B: methanol: dichloromethane = 1:1; Flow rate: |
| | fluorophenyl)-1-methyl-3- | | 20 mL/min; Gradient: 15% B to 15% B in 18 min; Wave |
| | ((1R,2R,5S)-4-methyl-8- | | Length: 220/254 nm. |
| | (1H-tetrazol-5-yl)-8- | | |
| | azabicyclo[3.2.1]octan-2- | | Chiral separation conditions for assumed cis isomers, 50A* |
| | yl)urea | | and 50D*: Column: CHIRALPAK ID, 2*25 cm, 5 μm; Mobile |
| 50″ | 1-(4-chloro-3- | 394.1 | Phase A: Hexanes (0.1% trifluoroacetic acid), Mobile Phase |
| * | fluorophenyl)-1-methyl-3- | | B: ethanol: dichloromethane = 1:1; Flow rate: 20 mL/min; |
| | ((1S,2S,5R)-4-methyl-8- | | Gradient: 25% B to 25% B in 11 min; Wave Length: 220/254 |
| | (1H-tetrazo1-5-yl)-8- | | nm. |
| | azabicyclo [3.2.1]octan-2- | | |
| | yl)urea | | |
| 50A | 1-(4-chloro-3- | 394.1 | Compound 50A*: Chiral RT (min) : 15.0 |
| * | fluorophenyl)-1-methyl-3- | | |
| | ((1R,2R,4R,5S)-4-methyl- | | |
| | 8-(1H-tetrazol-5-yl)-8- | | |
| | azabicyclo [3.2.1]octan-2- | | |
| | yl)urea | | |
| 50B | 1-(4-chloro-3- | 394.1 | Compound 50B*: Chiral RT (min) : 8.48 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 50C | 1-(4-chloro-3- | 394.1 | Compound 50C*: Chiral RT (min): 6.18 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 50D | 1-(4-chloro-3- | 394.1 | Compound 50D*: Chiral RT (min) : 16.62 |
| * | fluorophenyl)-1-methyl-3- | | |
| | ((1S,2S,4S,5R)-4-methyl-8- | | |
| | (1H-tetrazol-5-yl)-8- | | |
| | azabicyclo[3.2.1]octan-2- | | |
| | yl)urea | | |
| Rac-51 | 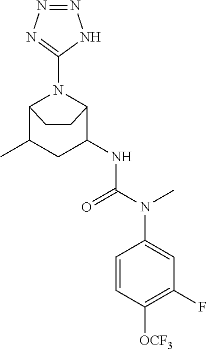 [SMILES: CC1CC(NC(=O)N(C)c2ccc(OC(F)(F)F)c(F)c2)C2CCC1N2c1nnn[nH]1] | 444.1 | Rac-51, 51′*, 51″*, 51A*, 51B*, 51C* and 51D* were prepared following Protocol B and Example 4 using 3-fluoro- N-methyl-4-(trifluoromethoxy)aniline as the aniline (iv) reagent and 4-methyl-8-(1H-tetrazol-5-yl)-8- azabicyclo[3.2.1]octan-2-amine as the amine (i) reagent. Rac-51 was then subjected to prep-HPLC using the following conditions: Column: Xbridge Prep OBD C18 Column, 30*150 mm, 5 μm; Mobile Phase A: Water(10 mmol/L NH₄HCO₃), Mobile Phase B: acetonitrile; Flow rate: 60 mL/min; Gradient: 22% B to 32% B in 10 min; Wave Length: 254 nm/220 nm to afford a mixture of compounds 51B* and 51C* as the first eluting peak, and a mixture of compounds 51A* and 51D* as the second eluting peak. |
| | 1-(3-fluoro-4- | | |
| | (trifluoromethoxy)phenyl)- | | |
| | 1-methyl-3-(4-methyl-8- | | |
| | (1H-tetrazol-5-yl)-8- | | |
| | azabicyclo[3.2.1]octan-2- | | Chiral separation conditions for assumed trans isomers, 51B* |
| | yl)urea | | and 51C*: Column: CHIRALPAK ID, 2*25 cm, 5 μm; Mobile |
| 51′* | 1-(3-fluoro-4- | 444.1 | Phase A: Hexanes (0.1% trifluoroacetic acid), Mobile Phase |
| | (trifluoromethoxy)phenyl)- | | B: ethanol: dichloromethane = 1:1; Flow rate: 20 mL/min; |
| | 1-methyl-3-((1R,2R,5S)-4- | | Gradient: 15% B to 15% B in 15.5 min; Wave Length: |
| | methyl-8-(1H-tetrazol-5- | | 220/254 nm. |
| | yl)-8- | | |
| | azabicyclo[3.2.1]octan-2- | | |
| | yl)urea | | |
| 51″ | 1-(3-fluoro-4- | 444.1 | Chiral separation conditions for assumed cis isomers, 51A* |
| * | (trifluoromethoxy)phenyl)- | | and 51D*: Column: CHIRAL ART Cellulose-SC, 2*25 cm, |
| | 1-methyl-3-((1S,2S,5R)-4- | | 5 μm; Mobile Phase A: Hexanes (0.1% trifluoroacetic acid), |
| | methyl-8-(1H-tetrazol-5- | | Mobile Phase B: ethanol: dichloromethane = 1:1; Flow rate: 20 |
| | yl)-8- | | mL/min; Gradient: 25% B to 25% B in 9.5 min; Wave Length: |
| | azabicyclo[3.2.1]octan-2- | | 220/254. |
| | yl)urea | | |
| 51A | 1-(3-fluoro-4- | 444.1 | Compound 51A*: Chiral RT (min): 7.31 |
| * | (trifluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 51B | 1-(3-fluoro-4- | 444.1 | Compound 51B*: Chiral RT(min): 8.34 |
| * | (trifluoromethoxy)phenyl)- | | |
| | 1-methyl-3-((1R,2S,4R,5S)- | | |
| | 4-methyl-8-(1H-tetrazol-5- | | |
| | yl)-8- | | |
| | azabicyclo[3.2.1]octan-2- | | |
| | yl)urea | | |
| 51C | 1-(3-fluoro-4- | 444.1 | Compound 51C*: Chiral RT(min): 13.92 |
| * | (trifluoromethoxy)phenyl)- | | |
| | 1-methyl-3-((1S,2R,4S,5R)- | | |
| | 4-methyl-8-(1H-tetrazol-5- | | |
| | yl)-8- | | |
| | azabicyclo[3.2.1]octan-2- | | |
| | yl)urea | | |
| 51D | 1-(3-fluoro-4- | 444.1 | Compound 51D*: Chiral RT (min): 8.82 |
| * | (trifluoromethoxy)phenyl)- | | |
| | 1-methyl-3-((1S,2S,4S,5R)- | | |
| | 4-methyl-8-(1H-tetrazol-5- | | |
| | yl)-8- | | |
| | azabicyclo[3.2.1]octan-2- | | |
| | yl)urea | | |
| Rac-52 | 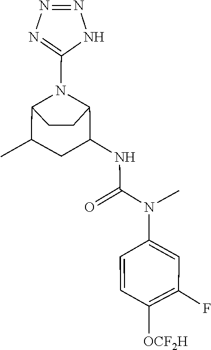 | 426.1 | Rac-52, 52′*, 52″*, 52A*, 52B*, 52C* and 52D* were prepared following Protocol B and Example 4 using 4- (difluoromethoxy)-3-fluoro-N-methylaniline as the aniline (iv) reagent and 4-methyl-8-(1H-tetrazol-5-yl)-8- azabicyclo[3.2.1]octan-2-amine as the amine (i) reagent. Rac-52 was then subjected to prep-HPLC using the following conditions: Column: XBridge Prep OBD C18 Column, 30*150 mm, 5 μm; Mobile Phase A: Water (50 mmol/L NH₄HCO₃), Mobile Phase B: acetonitrile; Flow rate: 60 mL/min; Gradient: 18% B to 28% B in 10 min, 23% B to 33% B in 15 min; Wave Length: 254 nm/220 nm to afford a mixture of compounds 52B* and 52C* as the first eluting peak, and a mixture of compounds 52A* and 52D* as the second eluting peak. Chiral separation conditions for assumed trans isomers, 52B* and 52C*: Column: CHIRALPAK IF, 5*25 cm, 5 μm; Mobile Phase A: Hexanes (0.1% trifluoroacetic acid), Mobile Phase B: ethanol: dichloromethane = 1:1; Flow rate: 20 mL/min; Gradient: isocratic 25; Wave Length: 220/254 nm. |
| | 1-(4-(difluoromethoxy)-3- | | |
| | fluorophenyl)-1-methyl-3- | | Chiral separation conditions for assumed cis isomers, 52A* |
| | (4-methyl-8-(1H-tetrazol-5- | | and 52D*: Column: Chiralpak IC, 2*25 cm, 5 μm; Mobile |
| | yl)-8- | | Phase A: Hexanes (0.2% trifluoroacetic acid), Mobile Phase |
| | azabicyclo[3.2.1]octan-2- | | B: ethanol: dichloromethane = 1:1; Flow rate: 20 mL/min; |
| | yl)urea | | Gradient: isocratic 25; Wave Length: 220/254 nm. |
| 52′* | 1-(4-(difluoromethoxy)-3- | 426.1 | |
| | fluorophenyl)-1-methyl-3- | | |
| | ((1R,2R,5S)-4-methyl-8- | | |
| | (1H-tetrazol-5-yl)-8- | | |
| | azabicyclo[3.2.1]octan-2- | | |
| | yl)urea | | |
| 52″ | 1-(4-(difluoromethoxy)-3- | 426.1 | |
| * | fluorophenyl)-1-methyl-3- | | |
| | ((1S,2S,5R)-4-methyl-8- | | |
| | (1H-tetrazol-5-yl)-8- | | |
| | azabicyclo[3.2.1]octan-2- | | |
| | yl)urea | | |
| 52A | 1-(4-(difluoromethoxy)-3- | 426.1 | Compound 52A*: Chiral RT (min): 9.25 |
| * | fluorophenyl)-1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 52B | 1-(4-(difluoromethoxy)-3- | 426.1 | Compound 52B*: Chiral RT(min): 5.85 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 52C | 1-(4-(difluoromethoxy)-3- | 426.1 | Compound 24C*: Chiral RT(min): 8.57 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 52D | 1-(4-(difluoromethoxy)-3- | 426.1 | Compound 52D*: Chiral RT (min): 11.59 |
| * | fluorophenyl)-1-methyl-3- | | |
| | ((1S,2S,4S,5R)-4-methyl-8- | | |
| | (1H-tetrazol-5-yl)-8- | | |
| | azabicyclo[3.2.1]octan-2- | | |
| | yl)urea | | |
| Rac-53 | 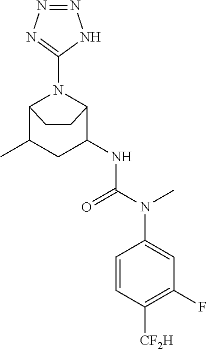 [SMILES: CC1CC(NC(=O)N(C)c2ccc(C(F)F)c(F)c2)C2CCC1N2c1nnn[nH]1] | 410.1 | Rac-53, 53′*, 53″*, 53A*, 53B*, 53C* and 53D* were prepared following Protocol B and Example 4 using 4- (difluoromethoxy)-3-fluoro-N-methylaniline as the aniline (iv) reagent and 4-methyl-8-(1H-tetrazol-5-yl)-8- azabicyclo[3.2.1]octan-2-amine as the amine (i) reagent. Rac-53 was then subjected to prep-HPLC using the following conditions: Column: Xselect CSH OBD Column 30*150 mm, 5 μm; Mobile Phase A: Water(0.05% trifluoroacetic acid), Mobile Phase B: acetonitrile; Flow rate: 60 mL/min; Gradient: 5% B to 5% B in 2 min, 25% B to 35% B in 10 min; Wave Length: 254/220 nm to afford a mixture of compounds 53B* and 53C* as the first eluting peak, and a mixture of compounds 53A* and 53D* as the second eluting peak. |
| | 1-(4-(difluoromethyl)-3- | | |
| | fluorophenyl)-1-methyl-3- | | |
| | (4-methyl-8-(1H-tetrazol-5- | | |
| | yl)-8- | | |
| | azabicyclo[3.2.1]octan-2- | | Chiral separation conditions for assumed trans isomers, 53B* |
| | yl)urea | | and 53C*: Column: CHIRALPAK IF, 2*25 cm, 5 μm; Mobile |
| 53′* | 1-(4-(difluoromethyl)-3- | 410.1 | Phase A: Hexanes (0.1% trifluoroacetic acid), Mobile Phase |
| | fluorophenyl)-1-methyl-3- | | B: ethanol: dichloromethane = 1:1; Flow rate: 20 mL/min; |
| | ((1R,2R,5S)-4-methyl-8- | | Gradient: 25% B to 25% B in 11.5 min; Wave Length: |
| | (1H-tetrazol-5-yl)-8- | | 220/254 nm. |
| | azabicyclo[3.2.1]octan-2- | | |
| | yl)urea | | |
| 53″ | 1-(4-(difluoromethyl)-3- | 410.1 | Chiral separation conditions for assumed cis isomers, 53A* |
| * | fluorophenyl)-1-methyl-3- | | and 53D*: Column: CHIRALPAK IC, 2*25 cm, 5 μm; Mobile |
| | ((1S,2S,5R)-4-methyl-8- | | Phase A: Hexanes (0.1% trifluoroacetic acid), Mobile Phase |
| | (1H-tetrazol-5-yl)-8- | | B: ethanol: dichloromethane = 1:1; Flow rate: 20 mL/min; |
| | azabicyclo[3.2.1]octan-2- | | Gradient: isocratic 25; Wave Length: 220/254 nm. |
| | yl)urea | | |
| 53A | 1-(4-(difluoromethyl)-3- | 410.1 | Compound 53A*: Chiral RT (min): 9.30 |
| * | fluorophenyl)-1-methyl-3- | | |
| | ((1R,2R,4R,5S)-4-methyl- | | |
| | 8-(1H-tetrazol-5-yl)-8- | | |
| | azabicyclo [3.2.1]octan-2- | | |
| | yl)urea | | |
| 53B | 1-(4-(difluoromethyl)-3- | 410.1 | Compound 53B*: Chiral RT (min): 10.62 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 53C | 1-(4-(difluoromethyl)-3- | 410.1 | Compound 53C*: Chiral RT (min): 7.21 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 53D | 1-(4-(difluoromethyl)-3- | 410.1 | Compound 53D*: Chiral RT (min): 11.50 |
| * | fluorophenyl)-1-methyl-3- | | |
| | ((1S,2S,4S,5R)-4-methyl-8- | | |
| | (1H-tetrazo1-5-yl)-8- | | |
| | azabicyclo[3.2.1]octan-2- | | |
| | yl)urea | | |
| Rac-54 | 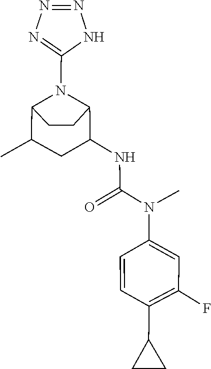 [SMILES: CC1CC(NC(=O)N(C)c2ccc(C3CC3)c(F)c2)C2CCC1N2c1nnn[nH]1] | 400.2 | Rac-54, 54′*, 54″*, 54A*, 54B*, 54C* and 54D* were prepared following Protocol B and Example 4 using 4- cyclopropyl-3-fluoro-N-methylbenzenamine as the aniline (iv) reagent and 4-methyl-8-(1H-tetrazol-5-yl)-8- azabicyclo[3.2.1]octan-2-amine as the amine (i) reagent. Rac-54 was then subjected to prep-HPLC using the following conditions: Column: Xselect CSH OBD Column 30*150 mm, 5 um; Mobile Phase A: Water (0.05% trifluoroacetic acid), Mobile Phase B: acetonitrile; Flow rate: 60 mL/min; Gradient: 5% B to 5% B in 2 min, 29% B to 39% B in 10 min; Wave Length: 254 nm/220 nm to afford a mixture of compounds 54B* and 54C* as the first eluting peak, and a mixture of compounds 54A* and 54D* as the second eluting peak. |
| | 1-(4-cyclopropyl-3- | | |
| | fluorophenyl)-1-methyl-3- | | |
| | (4-methyl-8-(1H-tetrazol-5- | | |
| | yl)-8- | | Chiral separation conditions for assumed trans isomers, 54B* |
| | azabicyclo[3.2.1]octan-2- | | and 54C*: Column: CHIRALPAK ID, 2*25 cm, 5 μm; Mobile |
| | yl)urea | | Phase A: Hexanes (0.1% trifluoroacetic acid), Mobile Phase |
| 54′* | 1-(4-cyclopropyl-3- | 400.2 | B: ethanol: dichloromethane = 1:1; Flow rate: 20 mL/min; |
| | fluorophenyl)-1-methyl-3- | | Gradient: 25% B to 25% B in 7.5 min; Wave Length: 220/254 |
| | ((1R,2R,5S)-4-methyl-8- | | nm. |
| | (1H-tetrazol-5-yl)-8- | | |
| | azabicyclo[3.2.1]octan-2- | | Chiral separation conditions for assumed cis isomers, 54A* |
| | yl)urea | | and 54D*: Column: CHIRAL ART Cellulose-SC, 2*25 cm, 5 |
| 54″ | 1-(4-cyclopropyl-3- | 400.2 | μm; Mobile Phase A: Hexanes (0.1% trifluoroacetic acid), |
| * | fluorophenyl)-1-methyl-3- | | Mobile Phase B: ethanol: dichloromethane = 1:1; Flow rate: 20 |
| | ((1S,2S,5R)-4-methyl-8- | | mL/min; Gradient: 25% B to 25% B in 16 min; Wave Length: |
| | (1H-tetrazol-5-yl)-8- | | 220/254 nm. |
| | azabicyclo [3.2.1]octan-2- | | |
| | yl)urea | | |
| 54A | 1-(4-cyclopropyl-3- | 400.2 | Compound 54A*: Chiral RT(min): 13.10 |
| * | fluorophenyl)-1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 54B | 1-(4-cyclopropyl-3- | 400.2 | Compound 54B*: Chiral RT(min): 6.53 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 54C | 1-(4-cyclopropyl-3- | 400.2 | Compound 54C*: Chiral RT(min): 5.11 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 54D | 1-(4-cyclopropyl-3- | 400.2 | Compound 54D*: Chiral RT(min): 6.53 |
| * | fluorophenyl)-1-methyl-3- | | |
| | ((1S,2S,4S,5R)-4-methyl-8- | | |
| | (1H-tetrazol-5-yl)-8- | | |
| | azabicyclo[3.2.1]octan-2- | | |
| | yl)urea | | |
| Rac-55 | 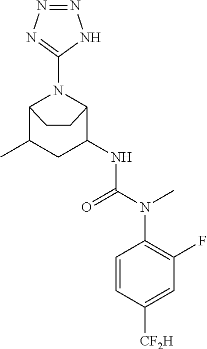 [SMILES: CC1CC(NC(=O)N(C)c2ccc(C(F)F)cc2F)C2CCC1N2c1nnn[nH]1] | 410.2 | Rac-55, 55′*, 55″*, 55A*, 55B*, 55C* and 55D* were prepared following Protocol B and Example 4 using 4- (difluoromethyl)-2-fluoro-N-methylbenzenamine as the aniline (iv) reagent and 4-methyl-8-(1H-tetrazol-5-yl)-8- azabicyclo[3.2.1] octan-2-amine as the amine (i) reagent. Rac-55 was then subjected to prep-HPLC using the following conditions: Column: XBridge Prep OBD C18 Column, 30*150 mm, 5 μm; Mobile Phase A: Water(10 mmol/L NH4HCO3), Mobile Phase B: acetonitrile ;Flow rate: 60 mL/min; Gradient: 19% B to 29% B in 10 min, 29% B to 36% B in 15 min; Wave Length: 254/220 nm to afford a mixture of compounds 55B* and 55C* as the first eluting peak, and a mixture of compounds 55A* and 55D* as the second eluting peak. |
| | 1-(4-(difluoromethyl)-2- | | |
| | fluorophenyl)-1-methyl-3- | | |
| | (4-methyl-8-(1H-tetrazol-5- | | |
| | yl)-8- | | |
| | azabicyclo[3.2.1]octan-2- | | Chiral separation conditions for assumed trans isomers, 55B* |
| | yl)urea | | and 55C*: Column: CHIRALPAK ID, 2*25 cm, 5 μm; Mobile |
| 55′* | 1-(4-(difluoromethyl)-2- | 410.2 | Phase A: Hexanes (0.1% trifluoroacetic acid), Mobile Phase |
| | fluorophenyl)-1-methyl-3- | | B: ethanol: dichloromethane = 1:1; Flow rate: 20 mL/min; |
| | ((1R,2R,5S)-4-methyl-8- | | Gradient: isocratic 20; Wave Length: 220/254 nm. |
| | (1H-tetrazol-5-yl)-8- | | |
| | azabicyclo[3.2.1]octan-2- | | Chiral separation conditions for assumed cis isomers, 55A* |
| | yl)urea | | and 55D*: Column: CHIRALPAK IG, 2*25 cm, 5 μm; Mobile |
| 55″ | 1-(4-(difluoromethyl)-2- | 410.2 | Phase A: Hexanes (0.1% trifluoroacetic acid), Mobile Phase |
| * | fluorophenyl)-1-methyl-3- | | B: ethanol: dichloromethane = 1:1; Flow rate: 20 mL/min; |
| | ((1S,2S,5R)-4-methyl-8- | | Gradient: 15% B to 15% B in 21 min; Wave Length: 220/254 |
| | (1H-tetrazol-5-yl)-8- | | nm. |
| | azabicyclo[3.2.1]octan-2- | | |
| | yl)urea | | |
| 55A | 1-(4-(difluoromethyl)-2- | 410.2 | Compound 55A*: Chiral RT(min): 19.31 |
| * | fluorophenyl)-1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 55B | 1-(4-(difluoromethyl)-2- | 410.2 | Compound 55B*: Chiral RT(min): 11.28 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 55C | 1-(4-(difluoromethyl)-2- | 410.2 | Compound 55C*: Chiral RT(min): 7.98 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 55D | 1-(4-(difluoromethyl)-2- | 410.2 | Compound 55D*: Chiral RT(min): 15.51 |
| * | fluorophenyl)-1-methyl-3- | | |
| | ((1S,2S,4S,5R)-4-methyl-8- | | |
| | (1H-tetrazol-5-yl)-8- | | |
| | azabicyclo[3.2.1]octan-2- | | |
| | yl)urea | | |
| Rac-56 | 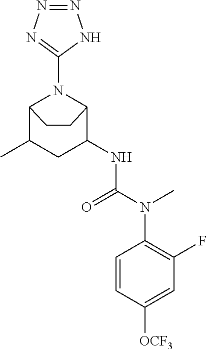 [SMILES: CC1CC(NC(=O)N(C)c2ccc(OC(F)(F)F)cc2F)C2CCC1N2c1nnn[nH]1] | 444.1 | Rac-56, 56′*, 56″*, 56A*, 56B*, 56C* and 56D* were prepared following Protocol B and Example 4 using 4- (trifluoromethoxy)-2-fluoro-N-methylbenzenamine as the aniline (iv) reagent and 4-methyl-8-(1H-tetrazol-5-yl)-8- azabicyclo[3.2.1]octan-2-amine as the amine (i) reagent. Rac-56 was then subjected to prep-HPLC using the following conditions: Column: XBridge Prep OBD C18 Column, 30*150 mm, 5 μm; Mobile Phase A: Water(10 mmol/L NH4HCO3), Mobile Phase B: acetonitrile; Flow rate: 60 mL/min; Gradient: 20% B to 30% B in 10 min, 30% B to 36% B in 15 min; Wave Length: 254/220 nm to afford a mixture of compounds 56B* and 56C* as the first eluting peak, and a mixture of compounds 56A* and 56D* as the second eluting peak. |
| | 1-(2-fluoro-4- | | |
| | (trifluoromethoxy)phenyl)- | | |
| | 1-methyl-3-(4-methyl-8- | | |
| | (1H-tetrazol-5-yl)-8- | | |
| | azabicyclo[3.2.1]octan-2- | | Chiral separation conditions for assumed trans isomers, 56B* |
| | yl)urea | | and 56C*: Column: CHIRALPAK ID, 2*25 cm, 5 μm; Mobile |
| 56′* | 1-(2-fluoro-4- | 444.1 | Phase A: Hexanes (0.1% trifluoroacetic acid), Mobile Phase |
| | (trifluoromethoxy)phenyl)- | | B: ethanol: dichloromethane = 1:1; Flow rate: 20 mL/min; |
| | 1-methyl-3-((1R,2R,5S)-4- | | Gradient: isocratic 25; Wave Length: 220/254 nm. |
| | methyl-8-(1H-tetrazol-5- | | |
| | yl)-8- | | Chiral separation conditions for assumed cis isomers, 56A* |
| | azabicyclo[3.2.1]octan-2- | | and 56D*: Column: Chiralpak IC, 2*25 cm, 5 μm; Mobile |
| | yl)urea | | Phase A: Hexanes (0.1% trifluoroacetic acid), Mobile Phase |
| 56″ | 1-(2-fluoro-4- | 444.1 | B: ethanol: dichloromethane = 1:1; Flow rate: 20 mL/min; |
| * | (trifluoromethoxy)phenyl)- | | Gradient: isocratic 25; Wave Length: 220/254 nm. |
| | 1-methyl-3-((1S,2S,5R)-4- | | Preparation of Compounds 56A* and 56D* is also provided in |
| | methyl-8-(1H-tetrazol-5- | | Example 6. |
| | yl)-8- | | |
| | azabicyclo[3.2.1]octan-2- | | |
| | yl)urea | | |
| 56A | 1-(2-fluoro-4- | 444.1 | Compound 56A*: Chiral RT (min): 11.11 |
| * | (trifluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 56B | 1-(2-fluoro-4- | 444.1 | Compound 56B*: Chiral RT (min): 6.37 |
| * | (trifluoromethoxy)phenyl)- | | |
| | 1-methyl-3-((1R,2S,4R,5S)- | | |
| | 4-methyl-8-(1H-tetrazol-5- | | |
| | yl)-8- | | |
| | azabicyclo[3.2.1]octan-2- | | |
| | yl)urea | | |
| 56C | 1-(2-fluoro-4- | 444.1 | Compound 56C*: Chiral RT (min): 4.91 |
| * | (trifluoromethoxy)phenyl)- | | |
| | 1-methyl-3-((1S,2R,4S,5R)- | | |
| | 4-methyl-8-(1H-tetrazol-5- | | |
| | yl)-8- | | |
| | azabicyclo[3.2.1]octan-2- | | |
| | yl)urea | | |
| 56D | 1-(2-fluoro-4- | 444.1 | Compound 56D*: Chiral RT (min): 13.17 |
| * | (trifluoromethoxy)phenyl)- | | |
| | 1-methyl-3-((1S,2S,4S,5R)- | | |
| | 4-methyl-8-(1H-tetrazol-5- | | |
| | yl)-8- | | |
| | azabicyclo[3.2.1]octan-2- | | |
| | yl)urea | | |
| Rac-57 | 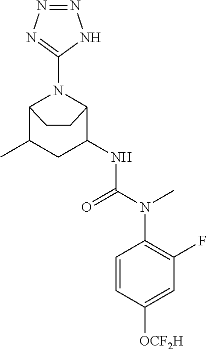 | 426.2 | Rac-57, 57′*, 57″*, 57A*, 57B*, 57C* and 57D* were prepared following Protocol B and Example 4 using 4- (difluoromethoxy)-2-fluoro-N-methylbenzenamine as the aniline (iv) reagent and 4-methyl-8-(1H-tetrazol-5-yl)-8- azabicyclo[3.2.1]octan-2-amine as the amine (i) reagent. Rac-57 was then subjected to prep-HPLC using the following conditions: Column: XBridge Prep OBD C18 Column, 30*150 mm, 5 μm; Mobile Phase A: Water (10 mmol/L NH₄HCO₃), Mobile Phase B: A acetonitrile;Flow rate: 60 mL/min; Gradient: 15% B to 25% B in 7.8 min; Wave Length: 254/226 nm to afford a mixture of compounds 57B* and 57C* as the first eluting peak, and a mixture of compounds 57A* and 57D* as the second eluting peak. Chiral separation conditions for assumed trans isomers, 57B* and 57C*: Column: CHIRALPAK IF, 2*25 cm, 5 μm; Mobile Phase A: Hexanes (0.1% trifluoroacetic acid), Mobile Phase B: ethanol: dichloromethane = 1:1; Flow rate: 20 mL/min; |
| | 1-(4-(difluoromethoxy)-2- | | |
| | fluorophenyl)-1-methyl-3- | | |
| | (4-methyl-8-(1H-tetrazol-5- | | |
| | yl)-8- | | |
| | azabicyclo[3.2.1]octan-2- | | |
| | yl)urea | | |
| 57′* | 1-(4-(difluoromethoxy)-2- | 426.2 | Gradient: 25% B to 25% B in 10 min; Wave Length: 220/254 |
| | fluorophenyl)-1-methyl-3- | | nm. |
| | ((1R,2R,5S)-4-methyl-8- | | Chiral separation conditions for assumed cis isomers, 57A* |
| | (1H-tetrazo1-5-yl)-8- | | and 57D*: Column: CHIRALPAK IC, 2*25 cm, 5 um; Mobile |
| | azabicyclo[3.2.1]octan-2- | | |
| | yl)urea | | |
| 57″ | 1-(4-(difluoromethoxy)-2- | 426.2 | Phase A: Hexanes (0.1% trifluoroacetic acid), Mobile Phase |
| * | fluorophenyl)-1-methyl-3- | | B: ethanol: dichloromethane = 1:1; Flow rate: 20 mL/min; |
| | ((1S,2S,5R)-4-methyl-8- | | Gradient: 25% B to 25% B in 13 min; Wave Length: 220/254 |
| | (1H-tetrazol-5-yl)-8- | | nm. |
| | azabicyclo[3.2.1]octan-2- | | |
| | yl)urea | | |
| 57A | 1-(4-(difluoromethoxy)-2- | 426.2 | Compound 57A*: Chiral RT (min): 9.26 |
| * | fluorophenyl)-1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 57B | 1-(4-(difluoromethoxy)-2- | 426.2 | Compound 57B*: Chiral RT (min): 8.08 |
| * | fluorophenyl)-1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 57C | 1-(4-(difluoromethoxy)-2- | 426.2 | Compound 57C*: Chiral RT (min): 5.63 |
| * | fluorophenyl)-1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 57D | 1-(4-(difluoromethoxy)-2- | 426.2 | Compound 57D*: Chiral RT (min): 11.13 |
| * | fluorophenyl)-1-methyl-3- | | |
| | ((1S,2S,4S,5R)-4-methyl-8- | | |
| | (1H-tetrazol-5-yl)-8- | | |
| | azabicyclo[3.2.1]octan-2- | | |
| | yl)urea | | |
| Rac-58 | 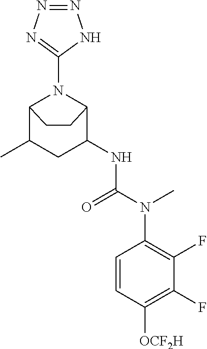 | 444.1 | Rac-58, 58′*, 58″*, 58A*, 58B*, 58C* and 58D* were prepared following Protocol B and Example 4 using 4- (difluoromethoxy)-2,3-difluoro-N-methylbenzenamine as the aniline (iv) reagent and 4-methyl-8-(1H-tetrazol-5-yl)-8- azabicyclo[3.2.1]octan-2-amine as the amine (i) reagent. Rac-58 was then subjected to prep-HPLC using the following conditions: Column: Xselect CSH OBD Column 30*150 mm, 5 μm; Mobile Phase A: Water(0.05% trifluoroacetic acid), Mobile Phase B: acetonitrile; Flow rate: 60 mL/min; Gradient: 5% B to 5% B in 2 min, 32% B to 42% B in 10 min; Wave Length: 254 nm/220 nm to afford a mixture of compounds 58B* and 58C* as the first eluting peak, and a mixture of compounds 58A* and 58D* as the second eluting peak. |
| | 1-(4-(difluoromethoxy)-2,3- | | |
| | difluorophenyl)-1-methyl- | | |
| | 3-(4-methyl-8-(1H-tetrazol- | | |
| | 5-yl)-8- | | |
| | azabicyclo[3.2.1]octan-2- | | Chiral separation conditions for assumed trans isomers, 58B* |
| | yl)urea | | and 58C*: Column: CHIRALPAK SC 2*25 cm, 5 μm; Mobile |
| 58′* | 1-(4-(difluoromethoxy)-2,3- | 444.1 | Phase A: Hexanes (0.1% trifluoroacetic acid), Mobile Phase |
| | difluorophenyl)-1-methyl- | | B: ethanol: dichloromethane = 1:1; Flow rate: 20 mL/min; |
| | 3-((1R,2R,5S)-4-methyl-8- | | Gradient: isocratic 25; Wave Length: 220/254 nm. |
| | (1H-tetrazol-5-yl)-8- | | |
| | azabicyclo[3.2.1]octan-2- | | Chiral separation conditions for assumed cis isomers, 58A* |
| | yl)urea | | and 58D*: Column: CHIRALPAK IF, 2*25 cm, 5 μm; Mobile |
| 58″ | 1-(4-(difluoromethoxy)-2,3- | 444.1 | Phase A: Hexanes (0.1% trifluoroacetic acid), Mobile Phase |
| * | difluorophenyl)-1-methyl- | | B: ethanol: dichloromethane = 1:1; Flow rate: 20 mL/min; |
| | 3-((1S,2S,5R)-4-methyl-8- | | Gradient: isocratic 15; Wave Length: 220/254 nm. |
| | (1H-tetrazol-5-yl)-8- | | |
| | azabicyclo[3.2.1]octan-2- | | |
| | yl)urea | | |
| 58A | 1-(4-(difluoromethoxy)-2,3- | 444.1 | Compound 58A*: Chiral RT(min): 7.80 |
| * | difluorophenyl)-1-methyl- | | |
| | 3-((1R,2R,4R,5S)-4- | | |
| | methyl-8-(1H-tetrazol-5- | | |
| | yl)-8- | | |
| | azabicyclo[3.2.1]octan-2- | | |
| | yl)urea | | |
| 58B | 1-(4-(difluoromethoxy)-2,3- | 444.1 | Compound 58B*: Chiral RT(min): 15.23 |
| * | difluorophenyl)-1-methyl- | | |
| | 3-((1R,2S,4R,5S)-4-methyl- | | |
| | 8-(1H-tetrazol-5-yl)-8- | | |
| | azabicyclo[3.2.1]octan-2- | | |
| | yl)urea | | |
| 58C | 1-(4-(difluoromethoxy)-2,3- | 444.1 | Compound 58C*: Chiral RT(min): 10.13 |
| * | difluorophenyl)-1-methyl- | | |
| | 3-((1S,2R,4S,5R)-4-methyl- | | |
| | 8-(1H-tetrazol-5-yl)-8- | | |
| | azabicyclo[3.2.1]octan-2- | | |
| | yl)urea | | |
| 58D | 1-(4-(difluoromethoxy)-2,3- | 444.1 | Compound 58D*: Chiral RT(min): 15.23 |
| * | difluorophenyl)-1-methyl- | | |
| | 3-((1S,2S,4S,5R)-4-methyl- | | |
| | 8-(1H-tetrazol-5-yl)-8- | | |
| | azabicyclo[3.2.1]octan-2- | | |
| | yl)urea | | |
| Rac-59 | 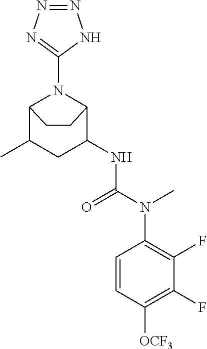 [SMILES: CC1CC(NC(=O)N(C)c2ccc(OC(F)(F)F)c(F)c2F)C2CCC1N2c1nnn[nH]1] | 462.1 | Rac-59, 59′*, 59″*, 59A*, 59B*, 59C* and 59D* were prepared following Protocol B and Example 4 using 4- (trifluoromethoxy)-2,3-difluoro-N-methylbenzenamine as the aniline (iv) reagent and 4-methyl-8-(1H-tetrazol-5-yl)-8- azabicyclo[3.2.1]octan-2-amine as the amine (i) reagent. Rac-59 was then subjected to prep-HPLC using the following conditions: Column: Xselect CSH OBD Column 30*150 mm, 5 um; Mobile Phase A: Water(0.05% trifluoroacetic acid), Mobile Phase B: acetonitrile;Flow rate: 60 mL/min; Gradient: 5% B to 5% B in 2 min, 35% B to 45% B in 10 min; Wave Length: 254/220 nm to afford a mixture of compounds 59B* and 59C* as the first eluting peak, and a mixture of compounds 59A* and 59D* as the second eluting peak. |
| | 1-(2,3-difluoro-4- | | |
| | (trifluoromethoxy)phenyl)- | | |
| | 1-methyl-3-(4-methyl-8- | | |
| | (1H-tetrazol-5-yl)-8- | | |
| | azabicyclo[3.2.1]octan-2- | | |
| | yl)urea | | Chiral separation conditions for assumed trans isomers, 59B* |
| 59′* | 1-(2,3-difluoro-4- | 462.1 | and 59C*: Column: CHIRALPAK IF, 2*25 cm, 5 μm; Mobile |
| | (trifluoromethoxy)phenyl)- | | Phase A: Hexanes (0.1% trifluoroacetic acid), Mobile Phase |
| | 1-methyl-3-((1R,2R,5S)-4- | | B: ethanol: dichloromethane = 1:1; Flow rate: 20 mL/min; |
| | methyl-8-(1H-tetrazol-5- | | Gradient: 15% B to 15% B in 18 min; Wave Length: 220/254 |
| | yl)-8- | | nm. |
| | azabicyclo[3.2.1]octan-2- | | |
| | yl)urea | | Chiral separation conditions for assumed cis isomers, 59A* |
| 59″ | 1-(2,3-difluoro-4- | 462.1 | and |
| * | (trifluoromethoxy)phenyl)- | | 59D*: Column: CHIRALPAK SC 2*25 cm, 5 μm; Mobile |
| | 1-methyl-3-((1S,2S,5R)-4- | | Phase A: Hexanes (0.1% trifluoroacetic acid), Mobile Phase |
| | methyl-8-(1H-tetrazol-5- | | B: ethanol: dichloromethane = 1:1; Flow rate: 20 mL/min; |
| | yl)-8- | | Gradient: isocratic 15; Wave Length: 220/254 nm. |
| | azabicyclo[3.2.1]octan-2- | | |
| | yl)urea | | |
| 59A | 1-(2,3-difluoro-4- | 462.1 | Compound 59A*: Chiral RT(min): 11.9 |
| * | (trifluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 59B | 1-(2,3-difluoro-4- | 462.1 | Compound 59B*: Chiral RT(min): 9.6 |
| * | (trifluoromethoxy)phenyl)- | | |
| | 1-methyl-3-((1R,2S,4R,5S)- | | |
| | 4-methyl-8-(1H-tetrazol-5- | | |
| | yl)-8- | | |
| | azabicyclo[3.2.1]octan-2- | | |
| | yl)urea | | |
| 59C | 1-(2,3-difluoro-4- | 462.1 | Compound 59C*: Chiral RT(min): 15.5 |
| * | (trifluoromethoxy)phenyl)- | | |
| | 1-methyl-3-((1S,2R,4S,5R)- | | |
| | 4-methyl-8-(1H-tetrazol-5- | | |
| | yl)-8- | | |
| | azabicyclo[3.2.1]octan-2- | | |
| | yl)urea | | |
| 59D | 1-(2,3-difluoro-4- | 462.1 | Compound 59D*: Chiral RT(min): 14.6 |
| * | (trifluoromethoxy)phenyl)- | | |
| | 1-methyl-3-((1S,2S,4S,5R)- | | |
| | 4-methyl-8-(1H-tetrazol-5- | | |
| | yl)-8- | | |
| | azabicyclo[3.2.1]octan-2- | | |
| | yl)urea | | |
| Rac-60 | 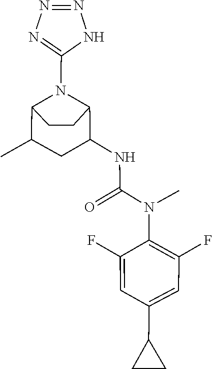 [SMILES: CC1CC(NC(=O)N(C)c2c(F)cc(C3CC3)cc2F)C2CCC1N2c1nnn[nH]1] | 418.1 | Rac-60, 60′*, 60″*, 60A*, 60B*, 60C* and 60D* were prepared following Protocol B and Example 4 using 4- cyclopropyl-2,6-difluoro-N-methylaniline as the aniline (iv) reagent and 4-methyl-8-(1H-tetrazol-5-yl)-8- azabicyclo[3.2.1]octan-2-amine as the amine (i) reagent. Rac-60 was then subjected to prep-HPLC using the following conditions: Column: XBridge Prep Phenyl OBD Column 19*250 mm, 5 μm; Mobile Phase A: Water(0.05% trifluoroacetic acid), Mobile Phase B: acetonitrile; Flow rate: 25 mL/min; Gradient: 35% B to 45% B in 10 min; Wave Length: 254/220 nm to afford a mixture of compounds 60B* and 60C* as the first eluting peak, and a mixture of compounds 60A* and 60D* as the second eluting peak. |
| | 1-(4-cyclopropyl-2,6- | | |
| | difluorophenyl)-1-methyl- | | Chiral separation conditions for assumed trans isomers, 60B* |
| | 3-(4-methyl-8-(1H-tetrazol- | | and 60C*: Column: CHIRALPAK IF, 2*25 cm, 5 μm; Mobile |
| | 5-yl)-8- | | Phase A: Hexanes (0.1% trifluoroacetic acid), Mobile Phase |
| | azabicyclo[3.2.1]octan-2- | | B: ethanol: dichloromethane = 1:1; Flow rate: 20 mL/min; |
| | yl)urea | | Gradient: 25% B to 25% B in 11.5 min; Wave Length: 220/254 nm. |
| 60′* | 1-(4-cyclopropyl-2,6- | 418.1 | |
| | difluorophenyl)-1-methyl- | | |
| | 3-((1R,2R,5S)-4-methyl-8- | | Chiral separation conditions for assumed cis isomers, 60A* |
| | (1H-tetrazol-5-yl)-8- | | and 60D*: Column: CHIRAL ART Cellulose-SC, 2*25 cm, 5 |
| | azabicyclo[3.2.1]octan-2- | | μm; Mobile Phase A: Hexanes (0.1% trifluoroacetic acid), |
| | yl)urea | | Mobile Phase B: ethanol: dichloromethane = 1:1; Flow rate: 20 |
| 60″ | 1-(4-cyclopropyl-2,6- | 418.1 | mL/min; Gradient: 25% B to 25% B in 15 min; Wave Length: |
| * | difluorophenyl)-1-methyl- | | 220/254 nm. |
| | 3-((1S,2S,5R)-4-methyl-8- | | |
| | (1H-tetrazol-5-yl)-8- | | |
| | azabicyclo[3.2.1]octan-2- | | |
| | yl)urea | | |
| 60A | 1-(4-cyclopropyl-2,6- | 418.1 | Compound 60A*: Chiral RT(min): 10.93 |
| * | difluorophenyl)-1-methyl- | | |
| | 3-((1R,2R,4R,5S)-4- | | |
| | methyl-8-(1H-tetrazol-5- | | |
| | yl)-8- | | |
| | azabicyclo[3.2.1]octan-2- | | |
| | yl)urea | | |
| 60B | 1-(4-cyclopropyl-2,6- | 418.1 | Compound 60B*: Chiral RT(min): 9.21 |
| * | difluorophenyl)-1-methyl- | | |
| | 3-((1R,2S,4R,5S)-4-methyl- | | |
| | 8-(1H-tetrazol-5-yl)-8- | | |
| | azabicyclo[3.2.1]octan-2- | | |
| | yl)urea | | |
| 60C | 1-(4-cyclopropyl-2,6- | 418.1 | Compound 60C*: Chiral RT(min): 6.71 |
| * | difluorophenyl)-1-methyl- | | |
| | 3-((1S,2R,4S,5R)-4-methyl- | | |
| | 8-(1H-tetrazol-5-yl)-8- | | |
| | azabicyclo[3.2.1]octan-2- | | |
| | yl)urea | | |
| 60D | 1-(4-cyclopropyl-2,6- | 418.1 | Compound 60D*: Chiral RT(min): 12.90 |
| * | difluorophenyl)-1-methyl- | | |
| | 3-((1S,2S,4S,5R)-4-methyl- | | |
| | 8-(1H-tetrazol-5-yl)-8- | | |
| | azabicyclo[3.2.1]octan-2- | | |
| | yl)urea | | |
| Rac-61 | 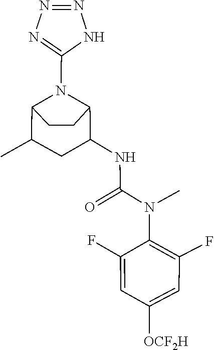 | 443.2 | Rac-61, 61′*, 61″*, 61A*, 61B*, 61C* and 61D* were prepared following Protocol B and Example 4 using 4- (difluoromethoxy)-2,6-difluoro-N-methylaniline as the aniline (iv) reagent and 4-methyl-8-(1H-tetrazol-5-yl)-8- azabicyclo[3.2.1]octan-2-amine as the amine (i) reagent. Rac-61 was then subjected to prep-HPLC using the following conditions: Column: XSelect CSH F-phenyl OBD Column 19*250 mm, 5 μm; Mobile Phase A: Water(0.05% trifluoroacetic acid), Mobile Phase B: can; Flow rate: 25 mL/min; Gradient: 28% B to 38% B in 10 min; Wave Length: 254/220 nm to afford a mixture of compounds 61B* and 61C* as the first eluting peak, and a mixture of compounds 61A* and 61D* as the second eluting peak. |
| | 1-(4-(difluoromethoxy)-2,6- | | |
| | difluorophenyl)-1-methyl- | | Chiral separation conditions for assumed trans isomers, 61B* |
| | 3-(4-methyl-8-(1H-tetrazol- | | and 61C*: Column: CHIRALPAK ID, 2*25 cm, 5 μm; Mobile |
| | 5-yl)-8- | | Phase A: Hexanes (0.1% TFA ) , Mobile Phase B: ethanol: |
| | azabicyclo[3.2.1]octan-2- | | dichloromethane = 1:1; Flow rate: 20 mL/min; Gradient: |
| | yl)urea | | isocratic 15; Wave Length: 220/254 nm. |
| 61′* | 1-(4-(difluoromethoxy)-2,6- | 443.2 | Chiral separation conditions for assumed trans isomers, 61B* |
| | difluorophenyl)-1-methyl- | | |
| | 3-((1R,2R,5S)-4-methyl-8- | | Chiral separation conditions for assumed cis isomers, 61A* |
| | (1H-tetrazol-5-yl)-8- | | and 61D*: Column: CHIRALPAK SB 2*25 cm, 5 μm; Mobile |
| | azabicyclo[3.2.1]octan-2- | | Phase A: Hexanes (0.1% trifluoroacetic acid), Mobile Phase |
| | yl)urea | | B: ethanol: dichloromethane = 1:1; Flow rate: 20 mL/min; |
| 61″ | 1-(4-(difluoromethoxy)-2,6- | 443.2 | Gradient: isocratic 10; Wave Length: 220/254 nm. |
| * | difluorophenyl)-1-methyl- | | |
| | 3-((1S,2S,5R)-4-methyl-8- | | |
| | (1H-tetrazol-5-yl)-8- | | |
| | azabicyclo[3.2.1]octan-2- | | |
| | yl)urea | | |
| 61A | 1-(4-(difluoromethoxy)-2,6- | 443.2 | Compound 61A*: Chiral RT(min): 19.35 |
| * | difluorophenyl)-1-methyl- | | |
| | 3-((1R,2R,4R,5S)-4- | | |
| | methyl-8-(1H-tetrazol-5- | | |
| | yl)-8- | | |
| | azabicyclo[3.2.1]octan-2- | | |
| | yl)urea | | |
| 61B | 1-(4-(difluoromethoxy)-2,6- | 443.2 | Compound 61B*: Chiral RT(min): 11.21 |
| * | difluorophenyl)-1-methyl- | | |
| | 3-((1R,2S,4R,5S)-4-methyl- | | |
| | 8-(1H-tetrazol-5-yl)-8- | | |
| | azabicyclo[3.2.1]octan-2- | | |
| | yl)urea | | |
| 61C | 1-(4-(difluoromethoxy)-2,6- | 443.2 | Compound 61C*: Chiral RT(min): 8.65 |
| * | difluorophenyl)-1-methyl- | | |
| | 3-((1S,2R,4S,5R)-4-methyl- | | |
| | 8-(1H-tetrazol-5-yl)-8- | | |
| | azabicyclo[3.2.1]octan-2- | | |
| | yl)urea | | |
| 61D | 1-(4-(difluoromethoxy)-2,6- | 443.2 | Compound 61D*: Chiral RT(min): 23.48 |
| * | difluorophenyl)-1-methyl- | | |
| | 3-((1S,2S,4S,5R)-4-methyl- | | |
| | 8-(1H-tetrazol-5-yl)-8- | | |
| | azabicyclo[3.2.1]octan-2- | | |
| | yl)urea | | |
| Rac-62 | 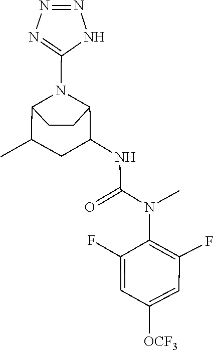 [SMILES: CC1CC(NC(=O)N(C)c2c(F)cc(OC(F)(F)F)cc2F)C2CCC1N2c1nnn[nH]1] | 462.1 | Rac-62, 62′*, 62″*, 62A*, 62B*, 62C* and 62D* were prepared following Protocol B and Example 4 using 4- (trifluoromethoxy)-2,6-difluoro-N-methylaniline as the aniline (iv) reagent and 4-methyl-8-(1H-tetrazol-5-yl)-8- azabicyclo[3.2.1]octan-2-amine as the amine (i) reagent. Rac-62 was then subjected to prep-HPLC using the following conditions: Column: XBridge Prep OBD C18 Column, 30*150 mm, 5 μm; Mobile Phase A: Water (10 mmol/L NH4HCO3), Mobile Phase B: acetonitrile; Flow rate: 60 mL/min; Gradient: 22% B to 32% B in 10 min, 32% B to 37% B in 15 min; Wave Length: 254/220 nm to afford a mixture of compounds 62B* and 62C* as the first eluting peak, and a mixture of compounds 62A* and 62D* as the second eluting peak. |
| | 1-(2,6-difluoro-4- | | |
| | (trifluoromethoxy)phenyl)- | | |
| | 1-methyl-3-(4-methyl-8- | | |
| | (1H-tetrazol-5-yl)-8- | | |
| | azabicyclo[3.2.1]octan-2- | | Chiral separation conditions for assumed trans isomers, 62B* |
| | yl)urea | | and 62C*: Column: CHIRALPAK IF, 2*25 cm, 5 μm; Mobile |
| 62′* | 1-(2,6-difluoro-4- | 462.1 | Phase A: Hexanes (0.1% trifluoroacetic acid), Mobile Phase |
| | (trifluoromethoxy)phenyl)- | | B: ethanol: dichloromethane = 1:1; Flow rate: 20 mL/min; |
| | 1-methyl-3-((1R,2R,5S)-4- | | Gradient: 15% B to 15% B in 15 min; Wave Length: 220/254 |
| | methyl-8-(1H-tetrazol-5- | | nm. |
| | yl)-8- | | |
| | azabicyclo[3.2.1]octan-2- | | Chiral separation conditions for assumed cis isomers, 62A* |
| | yl)urea | | and 62D*: Column: CHIRAL ART Cellulose-SC, 2*25 cm, 5 |
| 62″ | 1-(2,6-difluoro-4- | 462.1 | μm; Mobile Phase A: Hexanes (0.1% trifluoroacetic acid), |
| * | (trifluoromethoxy)phenyl)- | | Mobile Phase B: ethanol: dichloromethane = 1:1; Flow rate: 20 |
| | 1-methyl-3-((1S,2S,5R)-4- | | mL/min; Gradient: 15% B to 15% B in 18 min; Wave Length: |
| | methyl-8-(1H-tetrazol-5- | | 220/254 nm. |
| | yl)-8- | | |
| | azabicyclo[3.2.1]octan-2- | | |
| | yl)urea | | |
| 62A | 1-(2,6-difluoro-4- | 462.1 | Compound 62A*: Chiral RT(min): 13.53 |
| * | (trifluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 62B | 1-(2,6-difluoro-4- | 462.1 | Compound 62B*: Chiral RT(min): 13.31 |
| * | (trifluoromethoxy)phenyl)- | | |
| | 1-methyl-3-((1R,2S,4R,5S)- | | |
| | 4-methyl-8-(1H-tetrazol-5- | | |
| | yl)-8- | | |
| | azabicyclo[3.2.1]octan-2- | | |
| | yl)urea | | |
| 62C | 1-(2,6-difluoro-4- | 462.1 | Compound 62C*: Chiral RT(min): 9.12 |
| * | (trifluoromethoxy)phenyl)- | | |
| | 1-methyl-3-((1S,2R,4S,5R)- | | |
| | 4-methyl-8-(1H-tetrazol-5- | | |
| | yl)-8- | | |
| | azabicyclo[3.2.1]octan-2- | | |
| | yl)urea | | |
| 62D | 1-(2,6-difluoro-4- | 462.1 | Compound 62D*: Chiral RT(min): 16.48 |
| * | (trifluoromethoxy)phenyl)- | | |
| | 1-methyl-3-((1S,2S,4S,5R)- | | |
| | 4-methyl-8-(1H-tetrazol-5- | | |
| | yl)-8- | | |
| | azabicyclo[3.2.1]octan-2- | | |
| | yl)urea | | |
| Rac- 63 | 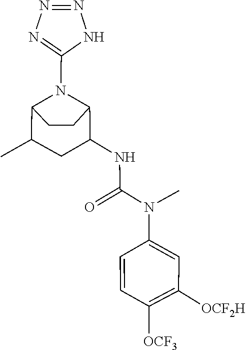 | 492.15 | Rac-63, 63′*, 63″*, 63A*, 63B*, 63C* and 63D* were prepared following Protocol B and Example 4 using 3- (difluoromethoxy)-N-methyl-4-(trifluoromethoxy)aniline as the aniline (iv) reagent and 4-methyl-8-(1H-tetrazol-5-yl)-8- azabicyclo[3.2.1]octan-2-amine as the amine (i) reagent. Rac-63 was then subjected to prep-HPLC using the following conditions: Column: XBridge Prep Phenyl OBD Column 19*250 mm; 5 μm; Mobile Phase A: Water(50 mmol/L NH₄HCO₃), Mobile Phase B: acetonitrile; Flow rate: 25 mL/min; Gradient: 30% B to 40% B in 10 min; Wave Length: 254 nm/220 nm to afford a mixture of compounds 63B* and 63C* as the first eluting peak, and a mixture of compounds 63A* and 63D* as the second eluting peak. |
| | 1-(3-(difluoromethoxy)-4- | | |
| | (trifluoromethoxy)phenyl)- | | |
| | 1-methyl-3-(4-methyl-8- | | |
| | (1H-tetrazol-5-yl)-8- | | |
| | azabicyclo [3.2.1]octan-2- | | Chiral separation conditions for assumed trans isomers, 63B* |
| | yl)urea | | and 63C*: Column: CHIRALPAK IF, 2*25 cm, 5 μm; Mobile |
| 63′* | 1-(3-(difluoromethoxy)-4- | 492.15 | Phase A: Hexanes (0.1% trifluoroacetic acid), Mobile Phase |
| | (trifluoromethoxy)phenyl)- | | B: ethanol: dichloromethane = 1:1; Flow rate: 20 mL/min; |
| | 1-methyl-3-((1R,2R,5S)-4- | | Gradient: 15% B to 15% B in 15 min; Wave Length: 220/254 |
| | methyl-8-(1H-tetrazol-5- | | nm. |
| | yl)-8- | | |
| | azabicyclo[3.2.1]octan-2- | | Chiral separation conditions for assumed cis isomers, 63A* |
| | yl)urea | | and 63D*: Column: CHIRAL ART Cellulose-SC, 2*25 cm, 5 |
| 63″ | 1-(3-(difluoromethoxy)-4- | 492.15 | μm; Mobile Phase A: Hexanes (0.1% trifluoroacetic acid), |
| * | (trifluoromethoxy)phenyl)- | | Mobile Phase B: ethanol: dichloromethane = 1:1; Flow rate: 20 |
| | 1-methyl-3-((1S,2S,5R)-4- | | mL/min; Gradient: 25% B to 25% B in 9 min; Wave Length: |
| | methyl-8-(1H-tetrazol-5- | | 220/254 nm. |
| | yl)-8- | | |
| | azabicyclo [3.2.1]octan-2- | | |
| | yl)urea | | |
| 63A | 1-(3-(difluoromethoxy)-4- | 492.15 | Compound 63A*: Chiral RT(min): 7.34 |
| * | (trifluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 63B | 1-(3-(difluoromethoxy)-4- | 492.15 | Compound 63B*: Chiral RT(min): 9.04 |
| * | (trifluoromethoxy)phenyl)- | | |
| | 1-methyl-3-((1R,2S,4R,5S)- | | |
| | 4-methyl-8-(1H-tetrazol-5- | | |
| | yl)-8- | | |
| | azabicyclo[3.2.1]octan-2- | | |
| | yl)urea | | |
| 63C | 1-(3-(difluoromethoxy)-4- | 492.15 | Compound 63C*: Chiral RT(min): 13.6 |
| * | (trifluoromethoxy)phenyl)- | | |
| | 1-methyl-3-((1S,2R,4S,5R)- | | |
| | 4-methyl-8-(1H-tetrazol-5- | | |
| | yl)-8- | | |
| | azabicyclo[3.2.1]octan-2- | | |
| | yl)urea | | |
| 63D | 1-(3-(difluoromethoxy)-4- | 492.15 | Compound 63D*: Chiral RT(min): 8.65 |
| * | (trifluoromethoxy)phenyl)- | | |
| | 1-methyl-3-((1S,2S,4S,5R)- | | |
| | 4-methyl-8-(1H-tetrazol-5- | | |
| | yl)-8- | | |
| | azabicyclo[3.2.1]octan-2- | | |
| | yl)urea | | |
| Rac-64 | 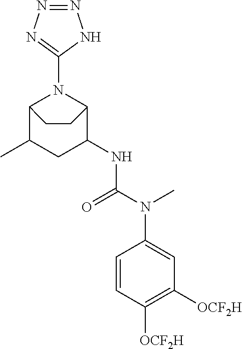 | 474.2 | Rac-64, 64′*, 64″*, 64A*, 64B*, 64C* and 64D* were prepared following Protocol B and Example 4 using 3,4- bis(difluoromethoxy)-N-methylaniline as the aniline (iv) reagent and 4-methyl-8-(1H-tetrazol-5-yl)-8- azabicyclo[3.2.1]octan-2-amine as the amine (i) reagent. Rac-64 was then subjected to prep-HPLC using the following conditions: Column: Xselect CSH OBD Column 30*150 mm, 5 μm; Mobile Phase A: Water(0.05% trifluoroacetic acid), Mobile Phase B: acetonitrile; Flow rate: 60 mL/min; Gradient: 5% B to 5% B in 2 min, 30% B to 40% B in 10 min, 40% B to 45% B in 13 min; Wave Length: 254/220 nm to afford a mixture of compounds 64B* and 64C* as the first eluting peak, and a mixture of compounds 64A* and 64D* as the second eluting peak. |
| | 1-(3,4- | | |
| | bis(difluoromethoxy)phenyl)- | | Chiral separation conditions for assumed trans isomers, 64B* |
| | 1-methyl-3-(4-methyl-8- | | and 64C*: Column: CHIRALPAK IF, 2*25 cm, 5 μm; Mobile |
| | (1H-tetrazol-5-yl)-8- | | Phase A: Hexanes (0.1% trifluoroacetic acid), Mobile Phase |
| | azabicyclo[3.2.1]octan-2- | | B: ethanol: dichloromethane = 1:1; Flow rate: 20 mL/min; |
| | yl)urea | | Gradient: isocratic 15; Wave Length: 220/254 nm. |
| 64′* | 1-(3,4- | 474.2 | |
| | bis(difluoromethoxy)phenyl)- | | |
| | 1-methyl-3-((1R,2R,5S)- | | |
| | 4-methyl-8-(1H-tetrazol-5- | | Chiral separation conditions for assumed cis isomers, 64A* |
| | yl)-8- | | and 64D*: Column: CHIRALPAK SC 2*25 cm, 5 μm; Mobile |
| | azabicyclo [3.2.1]octan-2- | | Phase A: Hexanes (0.1% trifluoroacetic acid), Mobile Phase |
| | yl)urea | | B: ethanol: dichloromethane = 1:1; Flow rate: 20 mL/min; |
| 64″ | 1-(3,4- | 474.2 | Gradient: isocratic 30; Wave Length: 220/254 nm. |
| * | bis(difluoromethoxy)phenyl)- | | |
| | 1-methyl-3-((1S,2S,5R)- | | |
| | 4-methyl-8-(1H-tetrazol-5- | | |
| | yl)-8- | | |
| | azabicyclo[3.2.1]octan-2- | | |
| | yl)urea | | |
| 64A | 1-(3,4- | 474.2 | Compound 64A*: Chiral RT(min): 6.557 |
| * | bis(difluoromethoxy)phenyl)- | | |
| | 1-methyl-3- | | |
| | ((1R,2R,4R,5S)-4-methyl- | | |
| | 8-(1H-tetrazol-5-yl)-8- | | |
| | azabicyclo[3.2.1]octan-2- | | |
| | yl)urea | | |
| 64B | 1-(3,4- | 474.2 | Compound 64B*: Chiral RT(min): 14.187 |
| * | bis(difluoromethoxy)phenyl)- | | |
| | 1-methyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 64C | 1-(3,4- | 474.2 | Compound 64C*: Chiral RT(min): 9.706 |
| * | bis(difluoromethoxy)phenyl)- | | |
| | 1-methyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 64D | 1-(3,4- | 474.2 | Compound 64D*: Chiral RT(min): 7.527 |
| * | bis(difluoromethoxy)phenyl)- | | |
| | 1-methyl-3- | | |
| | ((1S,2S,4S,5R)-4-methyl-8- | | |
| | (1H-tetrazol-5-yl)-8- | | |
| | azabicyclo [3.2.1]octan-2- | | |
| | yl)urea | | |
| Rac-65 | 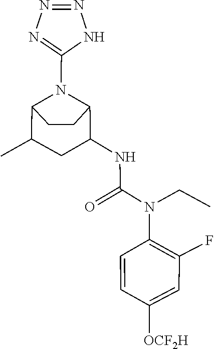 | — | Rac-65, 65′*, 65″*, 65B* and 65C* may be prepared following Protocol B and Example 4 using 4- (difluoromethoxy)-N-ethyl-2-fluoroaniline as the aniline (iv) reagent and 4-methyl-8-(1H-tetrazol-5-yl)-8- azabicyclo[3.2.1]octan-2-amine as the amine (i) reagent. |
| | 1-(4-(difluoromethoxy)-2- | | |
| | fluorophenyl)-1-ethyl-3-(4- | | |
| | methyl-8-(1H-tetrazol-5- | | |
| | yl)-8- | | |
| | azabicyclo[3.2.1]octan-2- | | |
| | yl)urea | | |
| 65′* | 1-(4-(difluoromethoxy)-2- | — | |
| | fluorophenyl)-1-ethyl-3- | | |
| | ((1R, 4R,5S)-4-methyl-8- | | |
| | (1H-tetrazol-5-yl)-8- | | |
| | azabicyclo[3.2.1]octan-2- | | |
| | yl)urea | | |
| 65″ | 1-(4-(difluoromethoxy)-2- | — | |
| * | fluorophenyl)-1-ethyl-3- | | |
| | ((1S, 4S, 5R)-4-methyl-8- | | |
| | (1H-tetrazol-5-yl)-8- | | |
| | azabicyclo[3.2.1]octan-2- | | |
| | yl)urea | | |
| 65B | 1-(4-(difluoromethoxy)-2- | — | |
| * | fluorophenyl)-1-ethyl-3- | | |
| | ((1R,2S,4R,5S)-4-methyl-8- | | |
| | (1H-tetrazol-5-yl)-8- | | |
| | azabicyclo[3.2.1]octan-2- | | |
| | yl)urea | | |
| 65C | 1-(4-(difluoromethoxy)-2- | — | |
| * | fluorophenyl)-1-ethyl-3- | | |
| | ((1S,2R,4S,5R)-4-methyl-8- | | |
| | (1H-tetrazol-5-yl)-8- | | |
| | azabicyclo[3.2.1]octan-2- | | |
| | yl)urea | | |
| 66 | (R)-3-(1-(1H-tetrazol-5- | — | Compound 66 was prepared following Protocol B and |
| | yl)piperidin-3-yl)-1-(4- | | Example 1 using 4-chloro-2-fluoroaniline as the aniline (iv) |
| | chloro-2-fluorophenyl)-1- | | reagent and (3R)-1-(1H-1,2,3,4-tetrazol-5-yl)piperidin-3- |
| | methylurea | | amine as the amine (i) reagent. |

### Biological Assay Methods

### Scintillation Proximity (SPA) NLRP3 Assay

The objective of this assay to is demonstrate binding of a test compound to human NLRP3 protein by displacement of 3H-MCC950, a selective inhibitor of NLRP3. Improved NLRP3 binding correlates with increased affinity for NLRP3 protein and, by extension, increased ability to interfere with its function.

SPA experiments were conducted in a total volume of 20 μL assay buffer containing 20 mM HEPES pH 7.5, 150 mM NaCl, 5 mM MgCl₂, 0.01% (v/v) Tween-20, 0.01% (w/v) bovine serum albumin (BSA) and 100 μM of adenosine diphosphate (ADP). A human NLRP3 protein (350 nM) (see, e.g., Sharif et al., Nature (2019) 570:338-343; Hochheiser et al., Nature (2022) 604:184-189; Adreeva et al., Cell (2021) 184:6299-6312; Wang et al., J Exp. Med. (2021) 219:e20211147) was incubated with various concentration of compounds in 5% dimethyl sulfoxide (DMSO) final for 15 minutes at room temperature in a white half-area 96 well OptiPlate (PerkinElmer #6002290) before adding 125 nM final of 3H-MCC950. After the addition of radiolabeled MCC950, the plate was incubated for 60 minutes at room temperature before adding 0.25 mg/ml of copper-coated polyvinyltoluene SPA beads (PerkinElmer #RPNQ0095). The plate was then transferred to a microplate counter (PerkinElmer 2450 Microbeta) to collect binding data. Binding data was analyzed 6 hrs post-PVT bead addition to allow sufficient time to reach equilibrium. Specific binding was calculated from averaged duplicates by subtracting signal from wells without NLRP3 protein.

SPA NLRP3 IC₅₀ assay results are provided in Table B. “A” activity=<1 uM; “B” activity=1-10 uM; “C” activity=>10-50 uM; “D” activity=>50 uM-100 uM; and “E” activity=>100 uM.

### Human PBMC NLRP3 Assay

The objective of this assay to is demonstrate if a test compound is able to interfere with human NLRP3 function in a cellular system.

Reagents: Human PBMCs (Normal): iXCells Cat #10HU-003; RPMI 1640 medium with GlutaMAX: ThermoFisher Cat #61870127 (Complete Media: 4.5 g/L D-glucose, 10% FBS, 100 mM NaPyr, 1% Pen/Strep, 10 mM HEPES and 0.05 mM of ®-mercaptoethanol; Assay Media: 4.5 g/L D-glucose, 100 mM NaPyr, 1% Pen/Strep, 10 mM HEPES and 0.05 mM of mercaptoethanol; 96-well V-bottom Plates: Costar Cat #3894; LPS (E. coli O26:B6): Sigma Cat #L2654, stock 5 mg/mL in PBS; ATP: Sigma Cat #A6419, prepared in a 250 mM stock in 1 M HEPES (adjusted to pH 7.4).

Cryopreserved PBMCs were rapidly thawed in a 37° C. water bath for 2 min. Cells were then centrifuged at 1200 RPM for 5 min and resuspended in ˜50 mL of fresh RPMI 1640 Complete Medium. A count was undertaken using a hemocytometer and adjusted to 2.5×10⁵ cells/mL. V-shaped 96-well plates were seeded with 200 μL of PBMCs (5×10⁴) per well and subsequently incubated overnight at 37° C. with 5% CO₂. Assay Media was then prepared containing 100 ng/ml of LPS. PBMCs were then centrifuged at 1,200 RPM for 5 min, serum containing media was aspirated, and 150 μL/well of Assay Media+LPS was immediately added. Assay Media without LPS was added in the untreated control wells. Cells were then primed with LPS for 4 h at 37° C. with 5% CO₂. A concentration response curve (CRC) was prepared of 1000× test compound in 100% DMSO. The CRC was then diluted 1:50 in Assay Media and then further diluted by 1:5× in Assay Media resulting in a final 4×CRC in 0.4% DMSO/Assay Media. 50 μL/well of 4× test compound CRC or vehicle (0.4% DMSO/Assay Media) was then transferred into each well and subsequently incubated for 30 min at 37° C. with 5% CO₂. Cells were stimulated by adding 4 μL of 250 mM ATP, using a 250 mM stock prepared in 1 M Hepes without further dilution (for a final concentration of 5 mM), for 1 h at 37° C. with 5% CO₂ Plates were then centrifuged at 1200 RPM for 5 min; and 50 μl of the media was transferred to a clean 96-well storage plate for cytokine measurements using the mesoscale platform, and stored at −80° C. until analyzed. Compounds were added 30 minutes before priming the cells with LPS when TNFα was required to be quantified in the cytokine panel to confirm selectivity.

PBMC NLRP3 IC₅₀ assay results, as a measurement of cellular potency, are provided in Table B. “A” activity=<0.01 uM; “B” activity=0.01-0.1 uM; “C” activity=>0.1-0.5 uM; “D” activity=>0.5 uM.

Human Whole Blood (hWB) NLRP3 Assay

The objective of this assay to is demonstrate if a test compound is able to interfere with human NLRP3 function in a whole blood system.

Human whole blood is drawn from healthy volunteers after obtaining written informed consent. Heparin lithium coated tubes are used to collect blood from volunteers. Blood samples are distributed on 96 well plates using 90 μl per well.

Priming procedure: Priming is performed by adding 5 μl of LPS (O26:B6; Sigma L-2654) at a final concentration of 1 μg/ml for 4.5 hours in a humidified incubator with 37° C., 5% CO₂. Thirty minutes prior to NLRP3 activation, 5 μl of a 20× compound solution or vehicle (2% dimethylsulfoxide (DMSO)) is added to each well and plates were incubated on a shaker (450 rpm) in a humidified incubator with 37° C., 5% CO₂.

Alternative priming procedure: Thirty minutes prior to LPS priming, 5 μl of a 20× compound solution or vehicle (2% dimethylsulfoxide (DMSO)) is added to each well and plates were incubated on a shaker (450 rpm) in a humidified incubator with 37° C., 5% CO₂. Priming is performed by adding 5 μl of LPS (O26:B6; Sigma L-2654) at a final concentration of 1 μg/ml for 5 hours in a humidified incubator with 37° C., 5% CO₂.

Activation is then performed by adding 3.3 μl of a 31×ATP solution per well. At the end of the 30 minutes stimulation, the plates are centrifuged (800 g, 10 min, room temperature) and the plasma from each well is frozen at −80° C. IL-1β levels in the supernatant were analyzed using a mesoscale discovery assay (MSD K151TUK) according to the manufacturers' instructions.

There is no detectable difference in assay results using the priming procedure versus the alternative priming procedure.

hWB NLRP3 IC₅₀ assay results, as a measurement of whole blood potency, are provided in Table B. “A” activity=<1 uM; “B” activity=1-2 uM; “C” activity=>2-3 uM; “D” activity=>3 uM.

Mouse Whole Blood (mWB) NLRP3 Assay

The objective of this assay to is demonstrate if a test compound is able to interfere with mouse NLRP3 function in a whole blood system.

Reagents: The following reagents were used: blood collection tubes (Heparin); U-bottom 96-well tissue culture (Falcon 353077); HBSS for LPS, ATP and compound dilutions (Gibco 24020-117); and LPS, E. coli serotype O26:B6 (Sigma L-2654).

Mouse IL-1b MSD assay: Blood was drawn from female CD1 mice (9 to 10 weeks) by cardiac puncture. Blood was plated (135 μL) per well in 96-well U-bottom plates. 7.5 μL of 20×LPS (20 μg/mL, final concentration of 1 μg/mL) was added and mixed by gentle pipetting, and the mixture was incubated in a TC incubator for five hours. 7.5 μL of 20× compound or vehicle per well was added and mixed by gentle pipetting. Compounds were diluted 1/50 in HBSS to prepare a 20× dilution curve in 2% DMSO. The mixture was incubated for 30 minutes in a TC incubator with shaking (450 rpm). 5 μl of 31×ATP (155 mM, final concentration of 5 mM) was added and mixed by gentle pipetting. The mixture was incubated in a TC incubator for 30 minutes with shaking (450 rpm). The plate was centrifuged for 10 minutes at 800×g and ˜70 μL of plasma was removed. Plasma was frozen if required. Mouse IL-1b was analyzed with IL-1b MSD assay (K152TUK).

Compounds were added 30 minutes before priming the cells with LPS when TNFα was required to be quantified in the cytokine panel to confirm selectivity.

mWB NLRP3 IC₅₀ assay results, as a measurement of whole blood potency, are provided in Table B. “A” activity=<1 uM; “B” activity=1-2 uM; “C” activity=>2-3 uM; “D” activity=>3 uM.

### Biological Assay Data

Various compounds described herein were tested according to the above-described assays. Dashed (--) line means no data available.

TABLE B
Activity data

| Cmp. | | | | |
|---|---|---|---|---|
| # | SPA IC₅₀ (UM) | PBMC IC₅₀ (uM) | mWB IC₅₀ (uM) | hWB IC₅₀ (uM) |
| 2A | B | C | — | — |
| 2B | — | — | — | — |
| 3A | E | — | — | — |
| 3B | — | — | — | — |
| 4A* | A | A | A | A |
| 4B* | B | — | — | — |
| 4C* | D | — | — | — |
| 4D* | B | — | — | — |
| 5A* | A | A | — | A |
| 5B* | B | — | — | — |
| 5C* | D | — | — | — |
| 5D* | B | — | — | — |
| 6A | A | B | — | — |
| 6B | — | — | — | — |
| 6C | — | — | — | — |
| 6D | — | — | — | — |
| 7A* | A | A | — | — |
| 7B* | B | — | — | — |
| 7C* | D | — | — | — |
| 7D* | C | — | — | — |
| 8A* | A | A | — | — |
| 8B* | B | — | — | — |
| 8C* | E | — | — | — |
| 8D* | B | — | — | — |
| 9A* | A | B | — | — |
| 9B* | B | — | — | — |
| 9C* | E | — | — | — |
| 9D | B | — | — | — |
| 10A* | A | A | A | A |
| 10B* | B | — | — | — |
| 10C* | E | — | — | — |
| 10D* | B | — | — | — |
| 11A* | A | A | A | A |
| 11B* | B | — | — | — |
| 11C* | D | — | — | — |
| 11D* | B | — | — | — |
| 12A* | A | B | — | C |
| 12B* | B | — | — | — |
| 12C* | C | — | — | — |
| 12D* | E | — | — | — |
| 13A* | A | A | — | A |
| 13B* | B | — | — | — |
| 13C* | E | — | — | — |
| 13D* | B | — | — | — |
| 14A* | A | B | — | B |
| 14B* | B | — | — | — |
| 14C* | E | — | — | — |
| 14D* | C | — | — | — |
| 15A* | A | A | — | A |
| 15B* | B | — | — | — |
| 15C* | E | — | — | — |
| 15D* | B | — | — | — |
| 16A* | A | A | — | B |
| 16B* | B | — | — | — |
| 16C* | D | — | — | — |
| 16D* | B | — | — | — |
| 17A* | A | A | — | B |
| 17B* | B | — | — | — |
| 17C* | B | — | — | — |
| 17D* | E | — | — | — |
| 18A* | A | A | — | A |
| 18B* | B | — | — | — |
| 18C* | C | — | — | — |
| 18D* | C | — | — | — |
| 19A* | A | A | — | A |
| 19B* | B | — | — | — |
| 19C* | E | — | — | — |
| 19D* | B | — | — | — |
| 20A* | A | A | — | A |
| 20B* | B | — | — | — |
| 20C* | C | — | — | — |
| 20D* | B | — | — | — |
| 21A* | A | B | — | A |
| 21B* | B | — | — | — |
| 21C* | E | — | — | — |
| 21D* | B | — | — | — |
| 22A* | A | — | — | B |
| 22B* | B | — | — | — |
| 22C* | C | — | — | — |
| 22D* | B | — | — | — |
| 23A* | A | A | — | A |
| 23B* | B | — | — | — |
| 23C* | C | — | — | — |
| 23D* | B | — | — | — |
| 24A* | A | A | — | A |
| 24B* | B | — | — | — |
| 24C* | C | — | — | — |
| 24D* | B | — | — | — |
| 25A* | A | — | — | B |
| 25B* | B | — | — | — |
| 25C* | B | — | — | — |
| 25D* | C | — | — | — |
| 26A* | A | B | — | D |
| 26B* | B | — | — | — |
| 26C* | E | — | — | — |
| 26D* | C | — | — | — |
| 27A* | B | — | — | — |
| 27B* | B | — | — | — |
| 27C* | C | — | — | — |
| 27D* | E | — | — | — |
| 28A* | B | — | — | — |
| 28B* | D | — | — | — |
| 28C* | D | — | — | — |
| 28D* | C | — | — | — |
| 29A* | B | — | — | — |
| 29B* | B | — | — | — |
| 29C* | D | — | — | — |
| 29D* | C | — | — | — |
| 30A* | E | — | — | — |
| 30B* | E | — | — | — |
| 30C* | E | — | — | — |
| 30D* | E | — | — | — |
| 31A* | C | — | — | — |
| 31B* | C | — | — | — |
| 31C* | C | — | — | — |
| 31D* | C | — | — | — |
| 32A* | C | — | — | D |
| 32B* | D | — | — | — |
| 32C* | D | — | — | — |
| 32D* | D | — | — | — |
| 33A* | C | — | — | — |
| 33B* | D | — | — | — |
| 33C* | D | — | — | — |
| 33D* | D | — | — | — |
| 34A | D | — | — | — |
| 34B | — | — | — | — |
| 34C | — | — | — | — |
| 34D | — | — | — | — |
| 35A | C | — | — | — |
| 35B | — | — | — | — |
| 35C | — | — | — | — |
| 35D | — | — | — | — |
| 36A | E | — | — | — |
| 36B | — | — | — | — |
| 36C | — | — | — | — |
| 36D | — | — | — | — |
| 37A | E | — | — | — |
| 37B | — | — | — | — |
| 37C | — | — | — | — |
| 37D | — | — | — | — |
| 38A | C | — | — | — |
| 38B | — | — | — | — |
| 38C | — | — | — | — |
| 38D | — | — | — | — |
| 39A | A | — | — | — |
| 39B | — | — | — | — |
| 39C | — | — | — | — |
| 39D | — | — | — | — |
| 40A* | A | B | — | B |
| 40B* | B | — | — | — |
| 40C* | C | — | — | — |
| 40D* | B | — | — | D |
| 41A* | A | A | — | A |
| 41B* | B | — | — | — |
| 41C* | D | — | — | — |
| 41D* | B | — | — | — |
| 42A* | A | A | — | A |
| 42B* | C | — | — | — |
| 42C* | E | — | — | — |
| 42D* | C | — | — | — |
| 43A* | A | A | — | A |
| 43B* | A | — | — | — |
| 43C* | C | — | — | — |
| 43D* | B | — | — | — |
| 44A* | A | — | — | C |
| 44B* | B | — | — | — |
| 44C* | C | — | — | — |
| 44D* | B | — | — | — |
| 45A* | B | — | — | A |
| 45B* | C | — | — | — |
| 45C* | D | — | — | — |
| 45D* | B | — | — | — |
| 46A* | A | A | — | A |
| 46B* | D | — | — | — |
| 46C* | C | — | — | — |
| 46D* | C | — | — | — |
| 47A* | A | — | — | B |
| 47B* | D | — | — | — |
| 47C* | E | — | — | — |
| 47D* | C | — | — | — |
| 48A* | A | — | — | A |
| 48B* | E | — | — | — |
| 48C* | D | — | — | — |
| 48D* | C | — | — | — |
| 49A* | A | B | — | B |
| 49B* | E | — | — | — |
| 49C* | D | — | — | — |
| 49D* | D | — | — | — |
| 50A* | B | — | — | — |
| 50B* | E | — | — | — |
| 50C* | E | — | — | — |
| 50D* | C | — | — | — |
| 51A* | A | — | — | B |
| 51B* | D | — | — | — |
| 51C* | E | — | — | — |
| 51D* | C | — | — | — |
| 52A* | A | B | — | A |
| 52B* | E | — | — | — |
| 52C* | E | — | — | — |
| 52D* | C | — | — | — |
| 53A* | B | — | — | — |
| 53B* | E | — | — | — |
| 53C* | E | — | — | — |
| 53D* | E | — | — | — |
| 54A* | A | A | — | A |
| 54B* | E | — | — | — |
| 54C* | E | — | — | — |
| 54D* | C | — | — | — |
| 55A* | A | — | — | A |
| 55B* | E | — | — | — |
| 55C* | D | — | — | — |
| 55D* | C | — | — | — |
| 56A* | A | — | — | A |
| 56B* | E | — | — | — |
| 56C* | D | — | — | — |
| 56D* | B | — | — | — |
| 57A* | A | A | — | A |
| 57B* | E | — | — | — |
| 57C* | D | — | — | — |
| 57D* | B | — | — | — |
| 58A* | — | — | — | A |
| 58B* | E | — | — | — |
| 58C* | D | — | — | — |
| 58D* | C | — | — | — |
| 59A* | A | C | — | C |
| 59B* | D | — | — | — |
| 59C* | E | — | — | — |
| 59D* | C | — | — | — |
| 60A* | A | A | — | A |
| 60B* | E | — | — | — |
| 60C* | D | — | — | — |
| 60D* | B | — | — | — |
| 61A* | A | A | — | A |
| 61B* | E | — | — | — |
| 61C* | E | — | — | — |
| 61D* | C | — | — | — |
| 62A* | A | B | — | A |
| 62B* | E | — | — | — |
| 62C* | D | — | — | — |
| 62D* | D | — | — | — |
| 63A* | A | — | — | C |
| 63B* | E | — | — | — |
| 63C* | D | — | — | — |
| 63D* | D | — | — | — |
| 64A* | A | B | — | A |
| 64B* | E | — | — | — |
| 64C* | E | — | — | — |
| 64D* | B | — | — | — |
| 65A* | A | — | — | A |
| 65B* | — | — | — | — |
| 65C* | — | — | — | — |
| 65D* | C | — | — | — |
| 66 | — | D | — | — |

### COMPARATIVE EXAMPLES

It has been found that compounds of Formula (I-B), which comprise anon-aromatic Ring B, as described herein, provide an improvement in activity compared to compounds with a smaller (5-membered) ring at the corresponding Ring B position. See, e.g., the data in Table C, demonstrating a >350-fold increase in potency as measured by PBMC IC₅₀ moving from Comparative Example A* (5-membered ring at the corresponding Ring B position) to Compound 2A (6-membered Ring B). Larger rings, e.g., 7-membered Ring B systems, are also well tolerated; compare, for example, the 5.9-fold increase in binding activity (SPA IC₅₀) moving from Compound 39A (6-membered Ring B) to Compound 11A* (7-membered Ring B).

It has further been found compounds of Formula (I-B), which comprise anon-aromatic Ring B, as described herein, provide an improvement in activity compared compounds with a phenyl ring at the corresponding Ring B position. See, e.g., the data in Table C, demonstrating a >15-fold increase in potency as measured by PBMC IC₅₀ moving from Comparative Example B2 to Compound 2A.

Inclusion of an ortho substituent on Ring A of compounds of Formula (I-B), e.g., for example wherein G₁ is CR^{G1} and R^{G1} is halo (e.g., fluoro or chloro), is also well tolerated. Compare, for example, the relatively similar potency of Compound 2A with no ortho substituent to Compound 66 comprising an ortho fluoro substituent, and Compound 42A* with no ortho substituent to Compound 57A* comprising an ortho fluoro substituent. Compare also, for example, the relatively similar potency for Compound 5A* comprising an ortho-fluoro substituent to Compound 7A* comprising an ortho-chloro substituent.

Various other substitutions on Ring A of Formula (I-B) are also well tolerated, e.g., for example wherein R¹ of Ring A is halo, C₁ haloalkyl, or —O(C₁haloalkyl). Compare, for example, the relatively similar potency for Compound 5A* comprising a para-chloro R¹ substituent to Compound 13A* comprising a para-trifluoromethyl R¹ substituent and to Compound 24A* comprising a para-trifluoromethoxy R¹ substituent. Compare also, for example, the relatively similar potency for Compound 4A* comprising a para-chloro R¹ substituent to Compound 10A* comprising a para-trifluoromethoxy R¹ substituent. Compare also, for example, the relatively similar potency for Compound 42A* comprising a para-difluoromethoxy R¹ substituent to Compound 45A* comprising a para-trifluoromethoxy R¹ substituent.

An ethylene bridging group on Ring B has been found to be also well tolerated; compare, e.g., the >12-fold improvement in binding activity (SPA IC₅₀) moving from Compound 39A (with no bridging group) to Compound 45A*.

Additional structural motifs present in compounds of Formula (I-B) and which have been found to improve activity include:
(i) a free amino (NH) moiety directly attached to Ring B
   a. Compare, e.g., the PBMC IC₅₀ data of Comparative Example C1 (where the amino moiety attached to Ring B is an N-methyl, and the corresponding R⁴ position is H) to Compound 2A (comprising a free NH moiety directly attached to Ring B, and wherein the R⁴ position is methyl) demonstrating a 40-fold improvement in potency;
   b. Compare, e.g., the PBMC IC₅₀ data of Comparative Example C2 (where the amino moiety attached to Ring B is an N-methyl, and the corresponding R⁴ position is also methyl) to Compound 2A (comprising a free NH moiety directly attached to Ring B, and wherein the R⁴ position is also methyl) demonstrating a >200-fold improvement in potency;
(ii) a substituted amino directly attached to Ring A at the R⁴ position
   a. Compare, e.g., the >15-fold improvement in binding activity (SPA IC₅₀) moving from Comparative Example B1 (wherein the corresponding Ring B is phenyl and the corresponding R⁴ substituent is H) to Comparative Example B2 (wherein the corresponding Ring B is phenyl the corresponding R⁴ substituent is methyl);
   b. Compare, e.g., the >19-fold improvement in binding activity (SPA IC₅₀) moving from Compound 27A* (where R⁴ is H) to Compound 5A* (where R⁴ is methyl);
   c. Compare also, e.g., the PBMC IC₅₀ data for Comparative Example G, Comparative Example J1 and Comparative Example J2, comprising no amino group directly attached to Ring A at to R⁴ position to Compound 2A comprising an N-Methyl R⁴ substituent, demonstrating a >350-fold increase in potency for each comparison;
   d. Compare, e.g., the PBMC IC₅₀ data for Comparative Example H, comprising an unsubstituted benzylic carbon at the corresponding N—R⁴ position to Compound 2A comprising an N-Methyl R⁴ substituent, demonstrating a 39-fold increase in potency; and
(iii) the presence of an unsubstituted tetrazolyl substituent attached to the Ring B nitrogen ring atom
   a. Compare, e.g., the PBMC IC₅₀ data for Comparative Example D1 and Comparative Example D2, each comprising a phenyl ring at the corresponding unsubstituted tetrazolyl position, to Compound 2A comprising an unsubstituted tetrazolyl substituent, demonstrating a respective 146-fold and >350-fold increase in potency; and
   b. Compare, e.g., the PBMC IC₅₀ data for Comparative Example F comprising an N-methylated tetrazolyl ring, to Compound 2A, comprising an unsubstituted tetrazolyl substituent, demonstrating a 58-fold increase in potency.

It has been further found that compounds of Formula (I-B), which comprise anon-aromatic Ring B and a non-hydrogen R⁴ group, as described herein, provide an improvement in activity compared to compounds with a corresponding phenyl ring at the Ring B position and a hydrogen at the corresponding R⁴ position. Compare, e.g., the SPA and PBMC IC₅₀ data in Table C, demonstrating a >19-fold improvement in binding activity and >18-fold improvement in potency moving from Comparative Example B1 to Compound 2A, and a >1950-fold improvement in binding activity and >700-fold improvement in potency moving from Comparative Example B1 to Compound 4A*.

TABLE C
Comparative Data

| | SPA IC₅₀ | PBMC | mWB | hWB |
|---|---|---|---|---|
| Compound | (uM) | IC₅₀ (UM) | IC₅₀ (UM) | IC₅₀ (uM) |
| 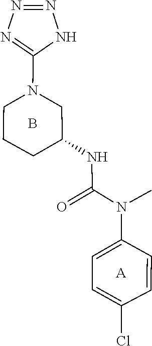 | 9.7 | 0.268 | — | — |
| Compound 2A | | | | |
| (R)-3-(1-(1H-tetrazol-5-yl)piperidin- | | | | |
| 3-yl)-1-(4-chlorophenyl)-1- | | | | |
| methylurea | | | | |
| 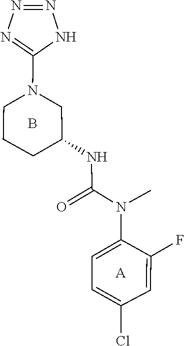 | — | 0.892 | — | — |
| Compound 66 | | | | |
| (R)-3-(1-(1H-tetrazol-5-yl)piperidin- | | | | |
| 3-yl)-1-(4-chloro-2-fluorophenyl)-1- | | | | |
| methylurea | | | | |
| 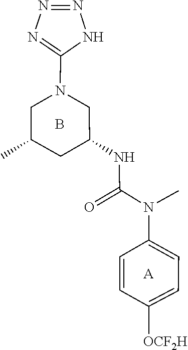 | 0.82 | — | — | — |
| Compound 39A | | | | |
| 1-(4-(difluoromethoxy)phenyl)-1- | | | | |
| methyl-3-((3R,5S)-5-methyl-1-(1H- | | | | |
| tetrazol-5-yl)piperidin-3-yl)urea | | | | |
| 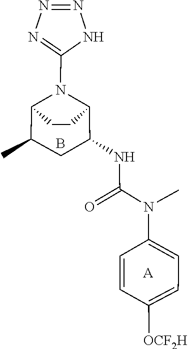 | 0.065 | 0.0041 | — | 0.29 |
| Compound 42A* | | | | |
| 1-(4-(difluoromethoxy)phenyl)-1- | | | | |
| methy1-3-((1R,2R,4R,5S)-4-methyl-8- | | | | |
| (1H-tetrazol-5-yl)-8- | | | | |
| azabicyclo[3.2.1]octan-2-yl)urea | | | | |
| 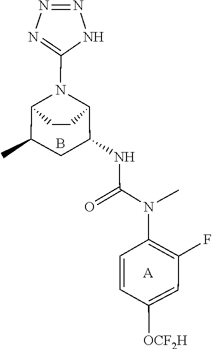 | 0.199 | 0.0031 | — | 0.19 |
| Compound 57A* | | | | |
| 1-(4-(difluoromethoxy)-2- | | | | |
| fluorophenyl)-1-methy1-3- | | | | |
| ((1R,2R,4R,5S)-4-methy1-8-(1H- | | | | |
| tetrazol-5-yl)-8- | | | | |
| azabicyclo[3.2.1]octan-2-yl)urea | | | | |
| 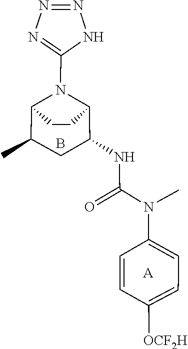 | 0.11 | — | — | 0.42 |
| Compound 45A* | | | | |
| 1-methy1-3-((1R,2R,4R,5S)-4-methyl- | | | | |
| 8-(1H-tetrazol-5-yl)-8- | | | | |
| azabicyclo[3.2.1]octan-2-yl)-1-(4- | | | | |
| (trifluoromethoxy)phenyl)urea | | | | |
| 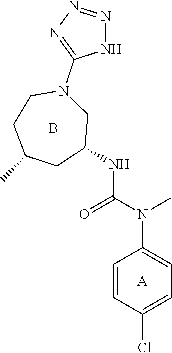 | 0.096 | 0.0068 | 0.040 | 0.15 |
| Compound 4A* | | | | |
| 1-(4-chlorophenyl)-1-methyl-3- | | | | |
| ((3R,5R)-5-methyl-1-(1H-tetrazol-5- | | | | |
| yl)azepan-3-yl)urea | | | | |
| 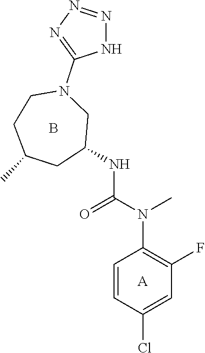 | 0.071 | 0.004 | — | 0.036 |
| Compound 5A* | | | | |
| 1-(4-chloro-2-fluorophenyl)-1-methyl- | | | | |
| 3-((3R,5R)-5-methyl-1-(1H-tetrazol- | | | | |
| 5-yl)azepan-3-yl)urea | | | | |
| 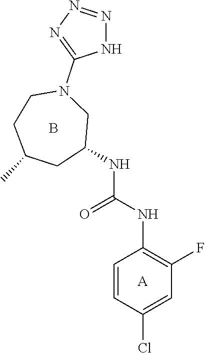 | 1.4 | — | — | — |
| Compound 27A* | | | | |
| 1-(4-chloro-2-fluorophenyl)-3- | | | | |
| ((3R,5R)-5-methyl-1-(1H-tetrazol-5- | | | | |
| yl)azepan-3-yl)urea | | | | |
| 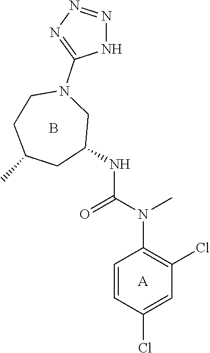 | 0.24 | 0.009 | — | — |
| Compound 7A* | | | | |
| 1-(2,4-dichlorophenyl)-1-methyl-3- | | | | |
| ((3R,5R)-5-methyl-1-(1H-tetrazol-5- | | | | |
| yl)azepan-3-yl)urea | | | | |
| 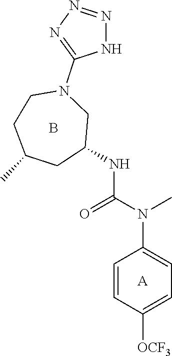 | 0.20 | 0.0091 | 0.095 | 0.30 |
| Compound 10A* | | | | |
| 1-methy1-3-((3R,5R)-5-methy1-1-(1H- | | | | |
| tetrazol-5-yl)azepan-3-yl)-1-(4- | | | | |
| (trifluoromethoxy)phenyl)urea | | | | |
| 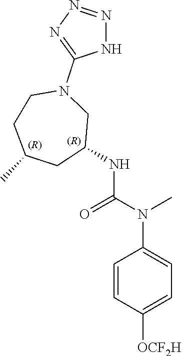 | 0.14 | 0.0042 | 0.026 | 0.058 |
| Compound 11A* | | | | |
| 1-(4-(difluoromethoxy)phenyl)-1- | | | | |
| methyl-3-((3R,5R)-5-methyl-1-(1H- | | | | |
| tetrazol-5-yl)azepan-3-yl)urea | | | | |
| 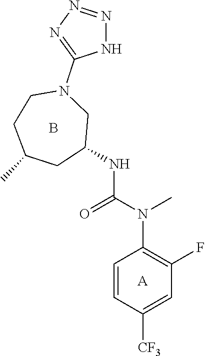 | 0.55 | 0.0056 | — | 0.20 |
| Compound 13A* | | | | |
| 1-(2-fluoro-4- | | | | |
| (trifluoromethyl)phenyl)-1-methyl-3- | | | | |
| ((3R,5R)-5-methyl-1-(1H-tetrazol-5- | | | | |
| yl)azepan-3-yl)urea | | | | |
| 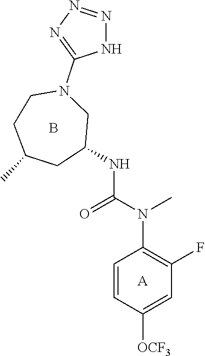 | 0.11 | 0.0079 | — | 0.26 |
| Compound 24A* | | | | |
| 1-(2-fluoro-4- | | | | |
| (trifluoromethoxy)phenyl)-1-methyl- | | | | |
| 3-((3R,5R)-5-methyl-1-(1H-tetrazol- | | | | |
| 5-yl)azepan-3-yl)urea | | | | |
| 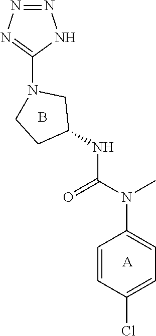 | — | >100 | — | — |
| Comparative Example A* | | | | |
| (R)-3-(1-(1H-tetrazol-5-yl)pyrrolidin- | | | | |
| 3-yl)-1-(4-chlorophenyl)-1- | | | | |
| methylurea | | | | |
| 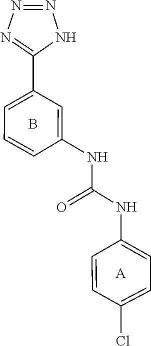 | 190 | >5 | — | — |
| Comparative Example B1 | | | | |
| 1-(3-(1H-tetrazol-5-yl)phenyl)-3-(4- | | | | |
| chlorophenyl)urea | | | | |
| CAS Reg. No. 925587-66-2 | | | | |
| 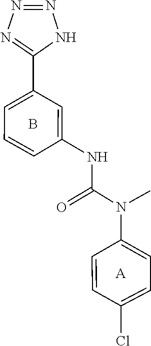 | 12.5 | 4.2 | — | — |
| Comparative Example B2 | | | | |
| 3-(3-(1H-tetrazol-5-yl)phenyl)-1-(4- | | | | |
| chlorophenyl)-1-methylurea | | | | |
| 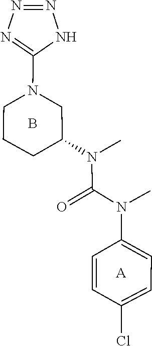 | — | 10.7 | — | — |
| Comparative Example C1 | | | | |
| (R)-1-(1-(1H-tetrazol-5-yl)piperidin- | | | | |
| 3-yl)-3-(4-chlorophenyl)-1- | | | | |
| methylurea | | | | |
| 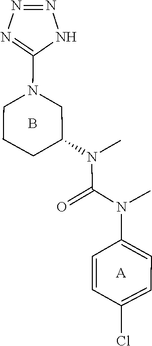 | — | 58.1 | — | — |
| Comparative Example C2 | | | | |
| (R)-1-(1-(1H-tetrazol-5-yl)piperidin- | | | | |
| 3-yl)-3-(4-chlorophenyl)-1,3- | | | | |
| dimethylurea | | | | |
| 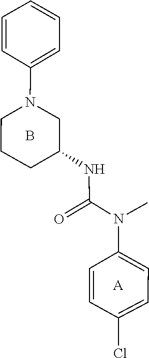 | — | 39.1 | — | — |
| Comparative Example D1 | | | | |
| (R)-1-(4-chlorophenyl)-1-methy1-3- | | | | |
| (1-phenylpiperidin-3-yl)urea | | | | |
| 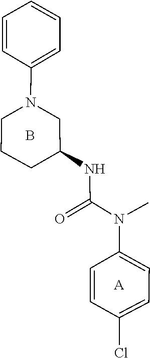 | — | >100 | — | — |
| Comparative Example D2 | | | | |
| (S)-1-(4-chlorophenyl)-1-methyl-3-(1- | | | | |
| phenylpiperidin-3-yl)urea | | | | |
| 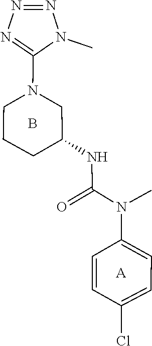 | — | 15.5 | — | — |
| Comparative Example F | | | | |
| (R)-1-(4-chlorophenyl)-1-methyl-3- | | | | |
| (1-(1-methy1-1H-tetrazol-5- | | | | |
| yl)piperidin-3-yl)urea | | | | |
| 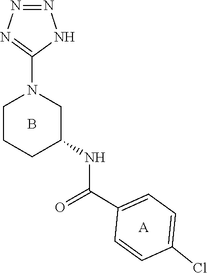 | — | >100 | — | — |
| Comparative Example G | | | | |
| (R)-N-(1-(1H-tetrazol-5-yl)piperidin- | | | | |
| 3-yl)-4-chlorobenzamide | | | | |
| 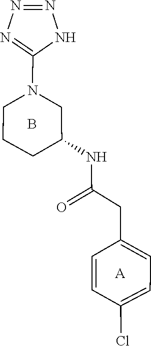 | — | 10.5 | — | — |
| Comparative Example H | | | | |
| (R)-N-(1-(1H-tetrazol-5-yl)piperidin- | | | | |
| 3-yl)-2-(4-chlorophenyl)acetamide | | | | |
| 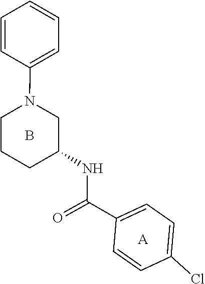 | — | >100 | — | — |
| Comparative Example J1** | | | | |
| (R)-4-chloro-N-(1-phenylpiperidin-3- | | | | |
| yl)benzamide | | | | |
| | — | >100 | — | — |
| Comparative Example J2** | | | | |
| (S)-4-chloro-N-(1-phenylpiperidin-3- | | | | |
| yl)benzamide | | | | |

**CAS Reg. No. 1625495-15-9 (racemate)

### OTHER EMBODIMENTS

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims or embodiments is introduced into another claim or embodiment. For example, any claim that is dependent on another claim can be modified to include one or more limitations found in any other claim that is dependent on the same base claim. Where the present disclosure recites elements presented as lists, e.g., in Markush group format, each subgroup of the elements is also disclosed, and any element(s) can be removed from the group. It should be further understood that when any variable (e.g., an R group) is present more than one time in a given Markush structure (e.g., 2 or more times), and that variable may be selected from a given list of two or more elements, unless otherwise stated or understood within the context of the present disclosure, that variable (e.g., the R group) at each repeated occurrence (e.g., 2 or more times) is independent of each other, being independently selected from that given list.

It should also be understood that, in general, where the present disclosure, or aspects of the present disclosure, is/are referred to as comprising particular elements and/or features, certain embodiments of the present disclosure or aspects of the present disclosure also consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are understood to be included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the present disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

The scope of the present embodiments described herein is not intended to be limited to the above Description, but also includes that as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I-B): 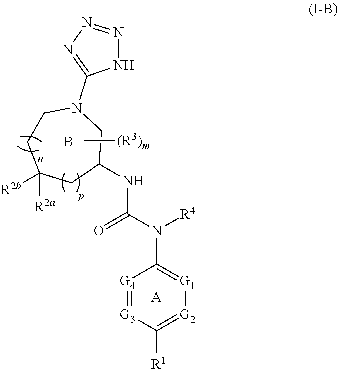
or a pharmaceutically acceptable salt or tautomer thereof;
wherein:
Ring A is a ring system wherein:
G₁ is CR^{G1} or N; G₂ is CR^{G2} or N; G₃ is CR^{G3} or N; and G₄ is CR^{G4} or N; provided no more than two of G₁, G₂, G₃, and G₄ are N;
R¹ is halo, C₁₋₆ alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆alkyl, C₁₋₆haloalkyl, —OR^{G5}, —SR^{G5}, or —N(R^{G5})₂,
or R¹ and G₂, together with the atoms to which they are attached, are joined to form a 5-membered heteroaryl ring independently substituted with 0, 1, 2, or 3 R^{G7};
R^{G1}, R^{G2}, R^{G3}, and R^{G4} are each independently selected from the group consisting of hydrogen, halo, C₁₋₆alkyl, C₁₋₆haloalkyl, and —OR^{G6}; and
R^{G5} and R^{G6} are each independently hydrogen, C₁₋₆ alkyl, or C₁₋₆ haloalkyl;
each instance of R^{G7} is independently halo, C₁₋₆ alkyl, C₁₋₆ haloalkyl, —OR^{G5}, —SR^{G5}, and —N(R^{G5})₂; and
Ring B is a ring system wherein:
n is 0 or 1;
p is 1 or 2;
m is 0, 1, 2, or 3;
each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2′} and R^{2b} are joined to form a C₃ carbocyclyl independently substituted with 0, 1, 2, or 3 halo;
each instance of R³ is independently halo, C₁₋₆ alkyl or C₁₋₆ haloalkyl, or two R³ groups are joined to form a C₁₋₃ alkylene bridging group or C₁₋₃ haloalkylene bridging group; and
R⁴ is hydrogen, C₁₋₃ alkyl, C₃-C₄ carbocyclyl, or C₃-C₄ carbocyclyl-C₁₋₃ alkyl-, wherein the alkyl and carbocyclyl are each independently substituted with 0, 1, 2, 3, 4, 5, or 6 halo, and wherein the carbocyclyl is further independently substituted with 0, 1, or 2 C₁₋₃ alkyl or C₁₋₃ haloalkyl.

2. The compound of claim 1, wherein the compound is of Formula: 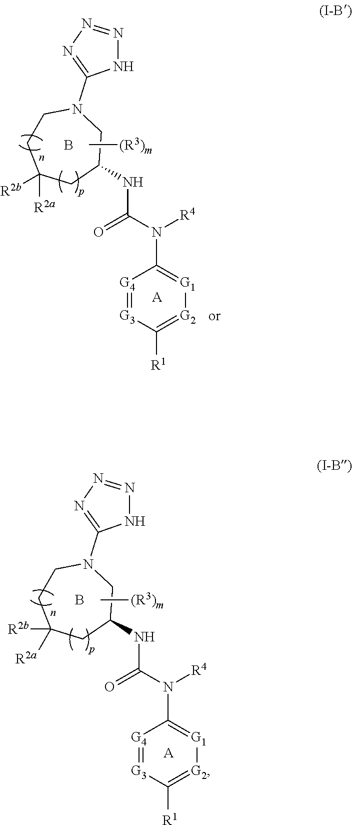
or a pharmaceutically acceptable salt or tautomer thereof.

3. The compound of either claim 1 or claim 2, wherein the compound is of Formula: 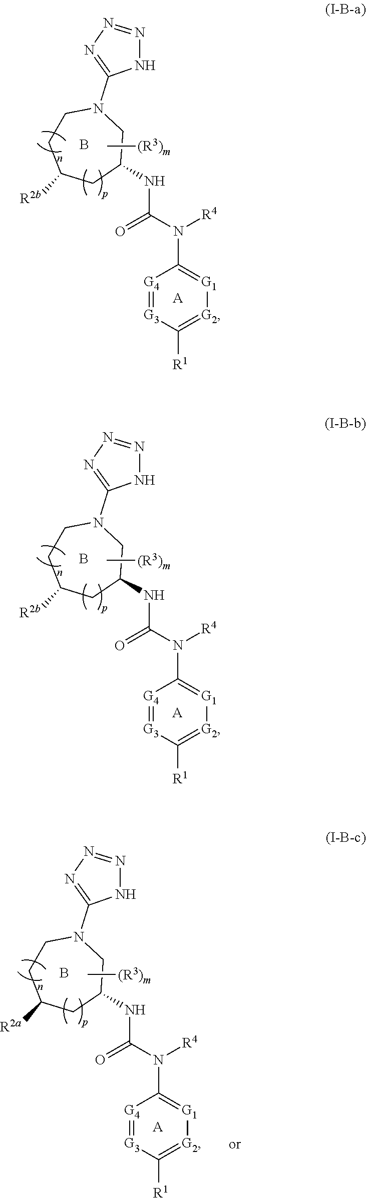 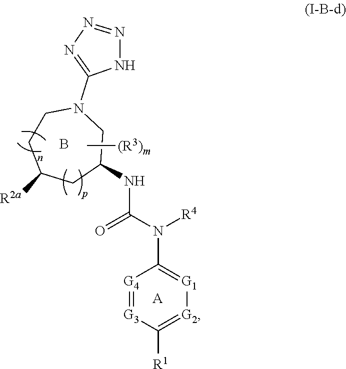
or a pharmaceutically acceptable salt or tautomer thereof.

4. The compound of claim 1, wherein the compound is of the Formula: 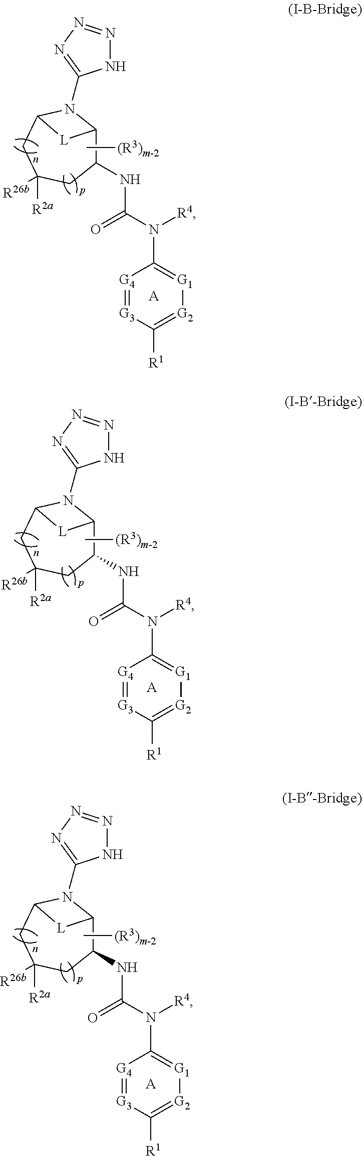 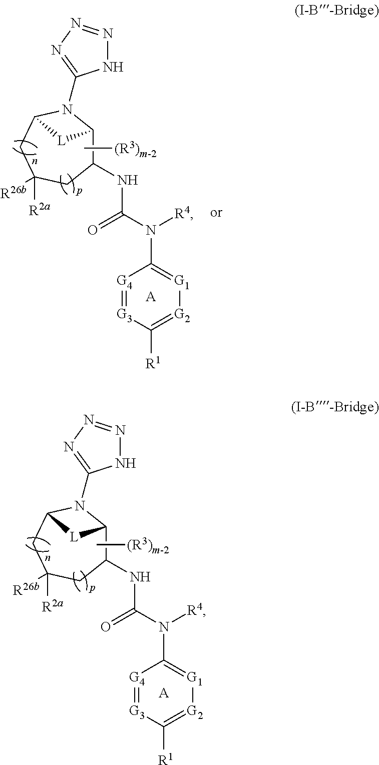
or a pharmaceutically acceptable salt or tautomer thereof, wherein L is a C₁₋₃ alkylene bridging group or C₁₋₃ haloalkylene bridging group.

5. The compound of claim 4, wherein the compound is of the Formula: 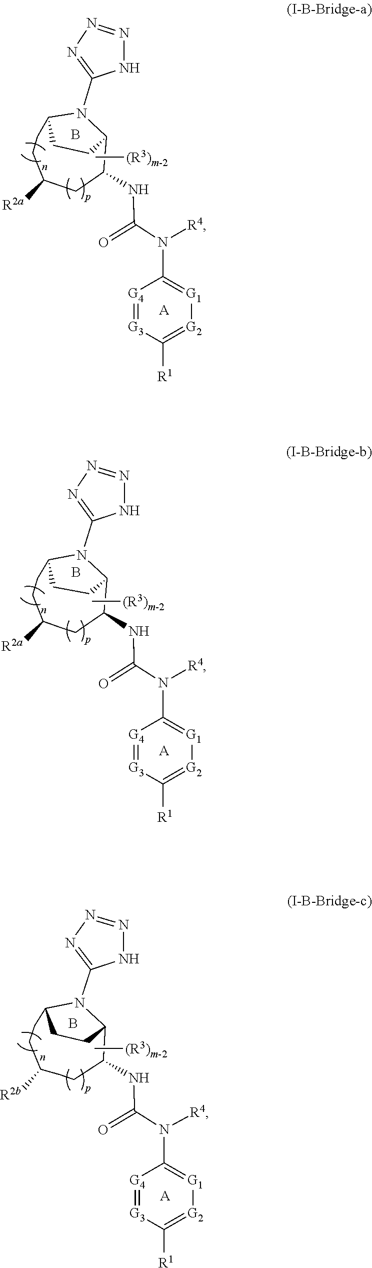 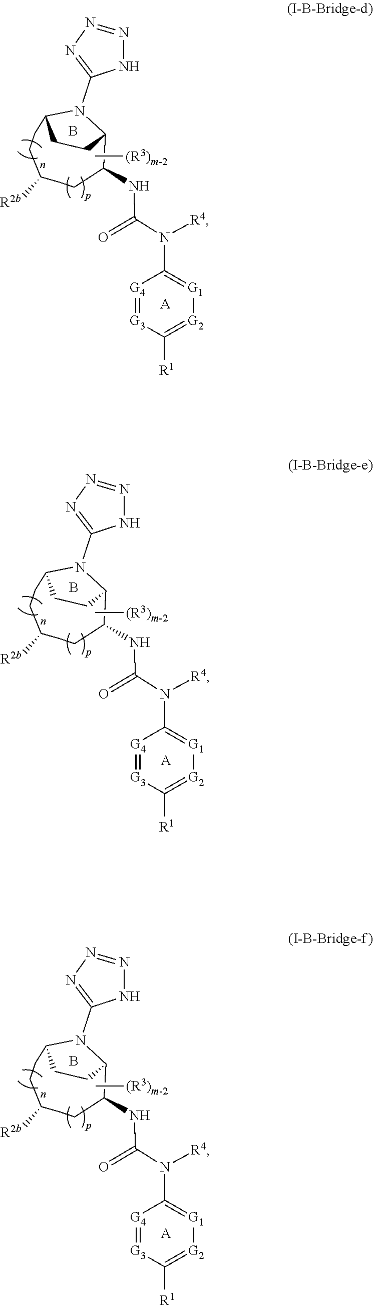 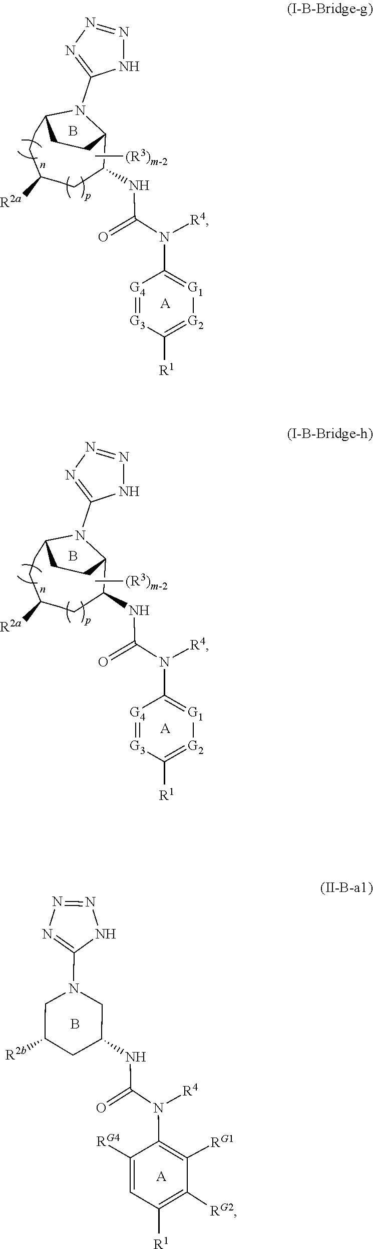 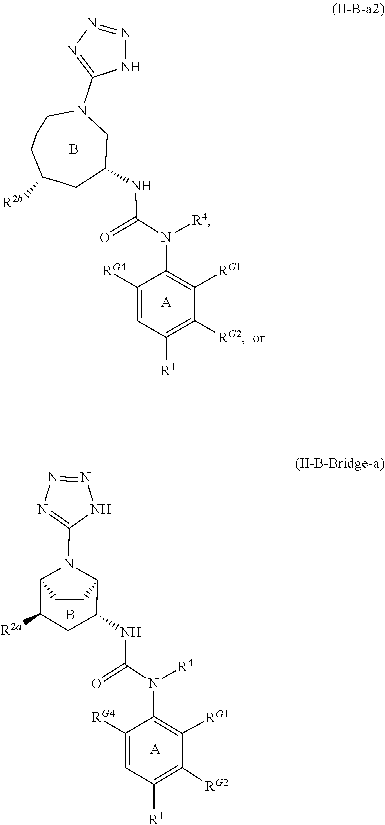
or a pharmaceutically acceptable salt or tautomer thereof.

6. The compound of any one of claims 1-5, or a pharmaceutically acceptable salt or tautomer thereof, wherein R⁴ is not hydrogen.

7. The compound of any one of claims 1-6, or a pharmaceutically acceptable salt or tautomer thereof, wherein Ring A is a ring system wherein:
R¹ is halo, C₁₋₆ haloalkyl, —OR^{G5}, or C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂,
or R¹ and G₂, together with the atoms to which they are attached, are joined to form a 5-membered heteroaryl ring independently substituted with 0, 1, 2, or 3 R^{G7};
R^{G1}, R^{G2}, R^{G3}, and R^{G4} are each independently selected from the group consisting of hydrogen, halo, and —OR^{G6};
R^{G5} and R^{G6} are each independently hydrogen or C₁₋₆ haloalkyl; and
each instance of R^{G7} is independently halo, C₁₋₆ alkyl, C₁₋₆ haloalkyl, —OR^{G5}, —SR^{G5}, and —N(R^{G5})₂.

8. The compound of claim 7, or a pharmaceutically acceptable salt or tautomer thereof, wherein Ring A is a ring system wherein:
R¹ is Cl, cyclopropyl, CF₃, CF₂H, OCF₃, or OCF₂H,
or R¹ and G₂, together with the atoms to which they are attached, are joined to form oxazole, isoxazole, pyrazole, or imidazole; and
R^{G1}, R^{G2}, R^{G3}, and R^{G4} are each independently selected from the group consisting of H, F, Cl, OH, and OCF₂H.

9. The compound of any one of claims 1-8, or a pharmaceutically acceptable salt or tautomer thereof, wherein Ring B is a ring system wherein:
each instance of R^{2a} and R^{2b} is independently hydrogen or C₁₋₆ alkyl;
two R³ groups are joined to form a C₁₋₃ alkylene bridging group; and
R⁴ is C₁₋₃ alkyl, C₃-C₄ carbocyclyl, or C₃-C₄ carbocyclyl-C₁₋₃ alkyl, wherein the alkyl and carbocyclyl are each independently substituted with 0, 1, 2, 3, 4, 5, or 6 halo.

10. The compound of claim 9, or a pharmaceutically acceptable salt or tautomer thereof, wherein Ring B is a ring system wherein:
each instance of R^{2a} and R^{2b} is independently hydrogen or methyl;
two R³ groups are joined to form an ethylene bridging group; and
R⁴ is methyl, ethyl, cyclopropyl, or cyclopropyl-methyl.

11. The compound of any one of claims 1-10, or a pharmaceutically acceptable salt or tautomer thereof, wherein:
G₁ is CR^{G1}, G₂ is CR^{G2}, G₃ is CR^{G3}, and G₄ is CR^{G4};
G₁ is N, G₂ is CR^{G2}, G₃ is CR^{G3}, and G₄ is CR^{G4};
G₁ is CR^{G1}, G₂ is N, G₃ is CR^{G3}, and G₄ is CR^{G4};
G₁ is CR^{G1}, G₂ is CR^{G2}, G₃ is N, and G₄ is CR^{G4};
G₁ is CR^{G1}, G₂ is CR^{G2}, G₃ is CR^{G3}, and G₄ is N;
G₁ is N, G₂ is CR^{G2}, G₃ is CR^{G3}, and G₄ is N;
G₁ is N, G₂ is CR^{G2}, G₃ is N, and G₄ is CR^{G4};
G₁ is CR^{G1}, G₂ is N, G₃ is N, and G₄ is CR^{G4}; or
G₁ is CR^{G1}, G₂ is CR^{G2}, G₃ is N, and G₄ is N.

12. The compound of claim 11, or a pharmaceutically acceptable salt or tautomer thereof, wherein:
G₁ is CH, G₂ is CH, G₃ is CH, and G₄ is CH;
G₁ is N, G₂ is CH, G₃ is CH, and G₄ is CH;
G₁ is CH, G₂ is N, G₃ is CH, and G₄ is CH;
G₁ is N, G₂ is CH, G₃ is CH, and G₄ is N;
G₁ is N, G₂ is CH, G₃ is N, and G₄ is CH; or
G₁ is CH, G₂ is CH, G₃ is N, and G₄ is N.

13. The compound of any one of claims 1-10, or a pharmaceutically acceptable salt or tautomer thereof, wherein G₁ is CR^{G1}, G₄ is CR^{G4}, and R¹ and G₂, together with the atoms to which they are attached, are joined to form a 5-membered heteroaryl ring independently substituted with 0, 1, 2, or 3 R^{G7}.

14. The compound of claim 13, or a pharmaceutically acceptable salt or tautomer thereof, wherein G₁ is CH and G₄ is CH.

15. The compound of claim any one of claims 1-14, or a pharmaceutically acceptable salt or tautomer hereof, wherein R¹ is —Cl, cyclopropyl, —CF₃, —CF₂H, —OCF₃, or —OCF₂H.

16. The compound of any one of claims 1-13, or a pharmaceutically acceptable salt or tautomer thereof, wherein R¹ and G₂, together with the atoms to which they are attached, are joined to form a 5-membered heteroaryl ring independently substituted with 0, 1, 2, or 3 R^{G7}.

17. The compound of claim 16, or a pharmaceutically acceptable salt or tautomer thereof, wherein R¹ and G₂, together with the atoms to which they are attached, are joined to form an oxazole, isoxazole, pyrazole, or imidazole.

18. The compound of any one of claims 1-9 and 12, or a pharmaceutically acceptable salt or tautomer thereof, wherein R^{G1}, R^{G2}, R^{G3}, and R^{G4} are each independently selected from the group consisting of hydrogen, F, Cl, OH, and OCF₂H.

19. The compound of claim 1, or a pharmaceutically acceptable salt or tautomer thereof, wherein R^{G5} is CF₃ or CF₂H, and R^{G6} is hydrogen or CF₂H.

20. The compound of any one of the preceding claims, or a pharmaceutically acceptable salt or tautomer thereof, wherein each instance of R^{2a} and R^{2b} is independently hydrogen or methyl.

21. The compound of any one of the preceding claims, or a pharmaceutically acceptable salt or tautomer thereof, wherein R⁴ is methyl, ethyl, cyclopropyl, or cyclopropyl-methyl.

22. The compound of any one of the preceding claims, or a pharmaceutically acceptable salt or tautomer thereof, wherein two R³ groups are joined to form an ethylene bridging group.

23. The compound of any one of the preceding claims, or a pharmaceutically acceptable salt or tautomer thereof, wherein Ring A of formula: 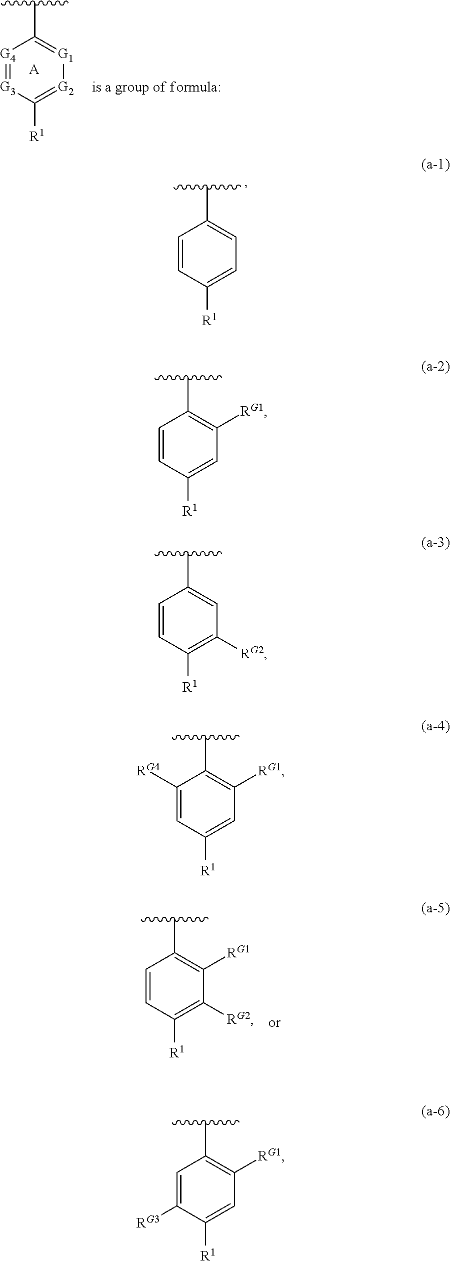
wherein R^{G1}, R^{G2}, R^{G3}, and R^{G4} are each independently selected from the group consisting of halo, C₁₋₆ alkyl, C₁₋₆ haloalkyl, and —OR^{G6}.

24. The compound of claim 23, or a pharmaceutically acceptable salt or tautomer thereof, wherein Ring A of formula (a-2), (a-4), (a-5), (a-6) is a group of formula:

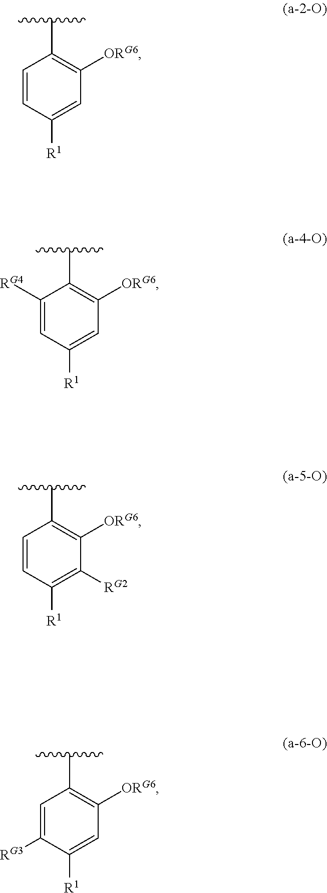

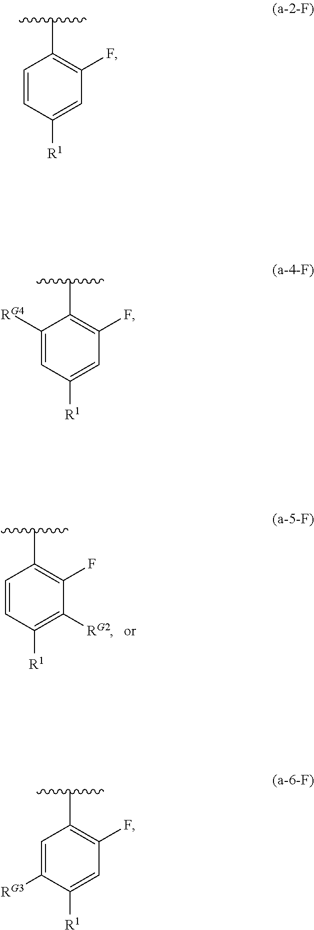

25. The compound of any one of claims 1-22, or a pharmaceutically acceptable salt or tautomer thereof, wherein Ring A of formula: 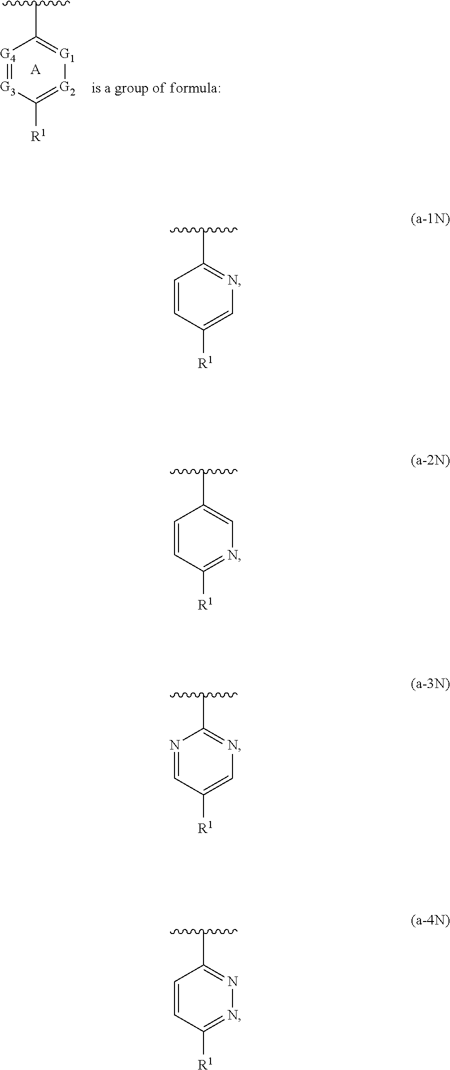 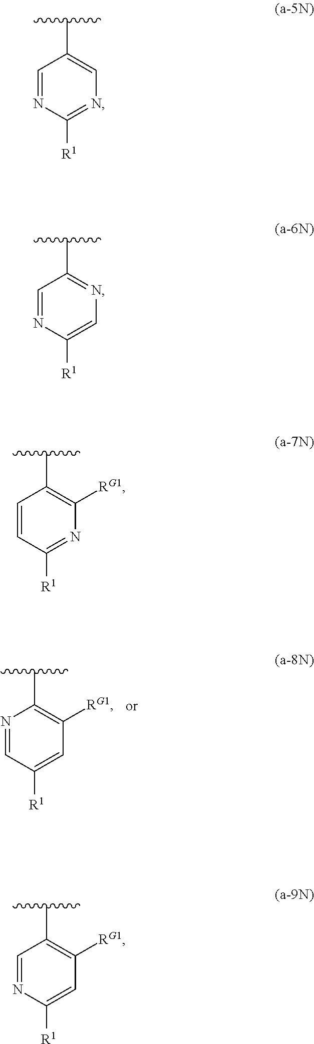
wherein R^{G1} is halo, C₁₋₆alkyl, C₁₋₆haloalkyl, or —OR^{G6}.

26. The compound of claim 25, or a pharmaceutically acceptable salt or tautomer thereof, wherein Ring A of formula (a-7N), (a-8N), or (a-9N) is of the formula:

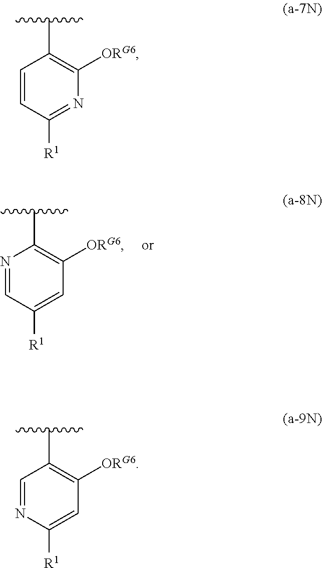

27. The compound of any one of claims 1-22, or a pharmaceutically acceptable salt or tautomer thereof, wherein Ring A of formula: 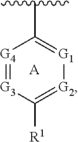
and R¹ and G₂, together with the atoms to which they are attached, are joined to form a 5-membered heteroaryl ring, wherein the Ring A, R¹, and G² provide a group of formula: 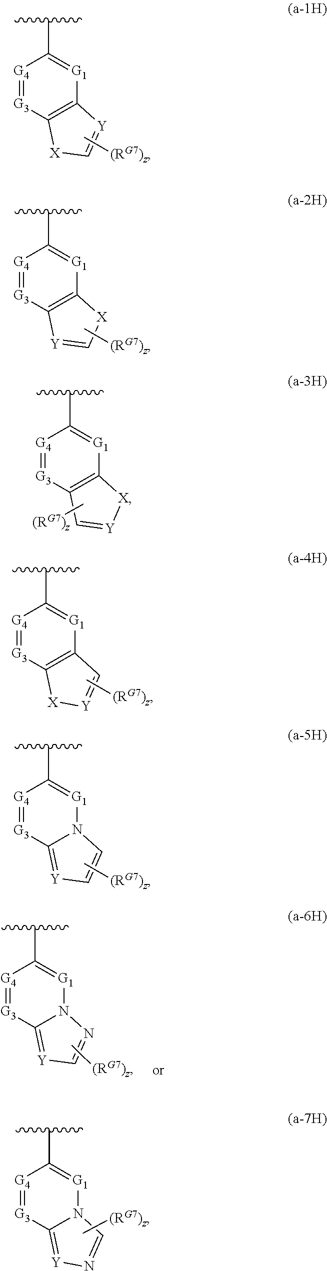
wherein:
X is O, S, NH, or NR^{G7};
Y is N, CH, or CR^{G7}; and
z is 0 or 1;
provided if R^{G7} is a group attached to a nitrogen (N) atom, then R^{G7} is C₁₋₆ alkyl or C₁₋₆ haloalkyl.

28. The compound of claim 27, or a pharmaceutically acceptable salt or tautomer thereof, wherein Ring A, R¹, and G₂, together with the atoms to which they are attached, are joined to form a 5-membered heteroaryl ring, wherein the group is of formula:

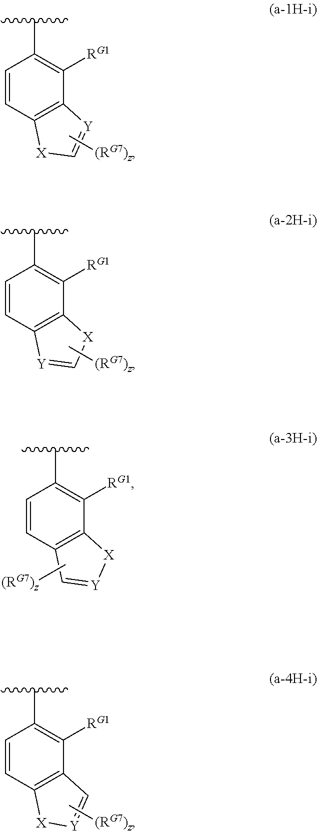

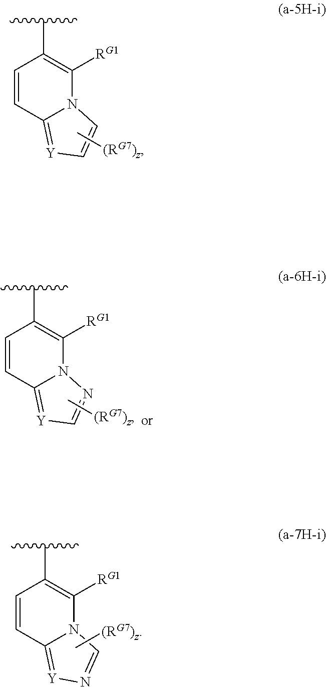

29. The compound of any one of the preceding claims, or a pharmaceutically acceptable salt or tautomer thereof, wherein Ring A is a group of formula:

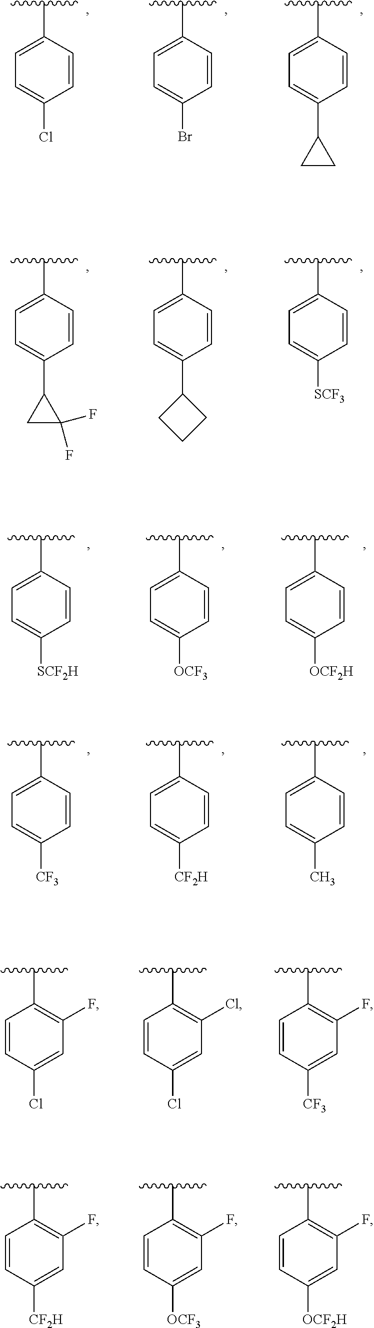

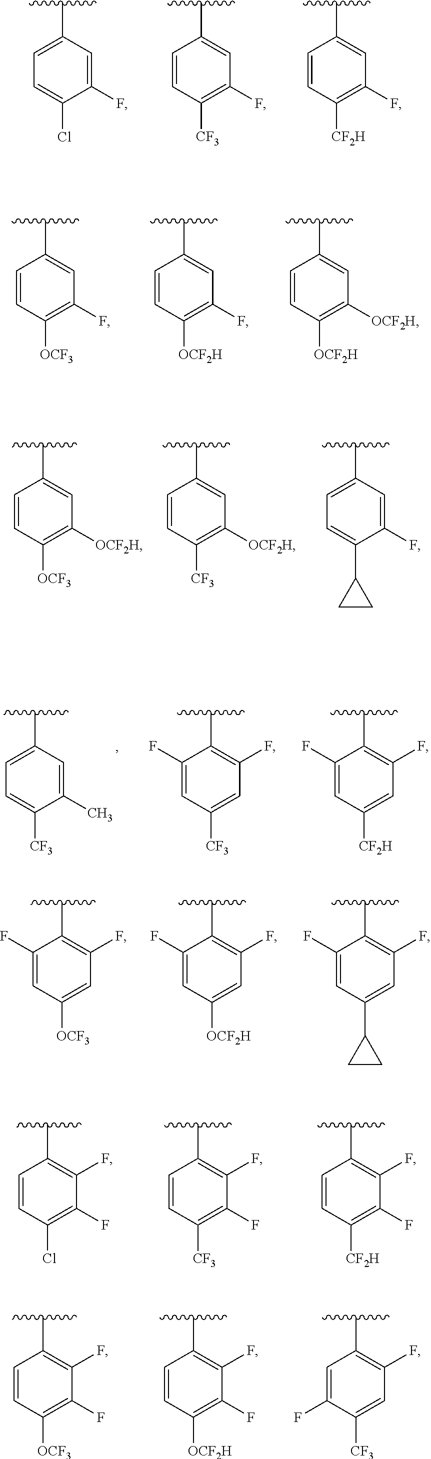

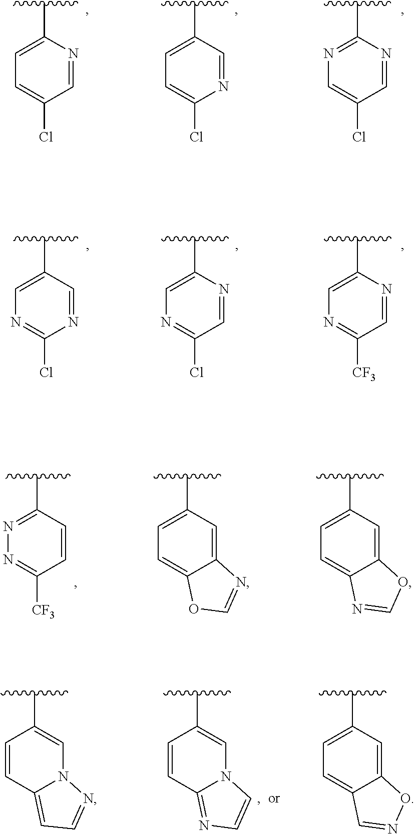

30. The compound of any one of claims 1-28, or a pharmaceutically acceptable salt or tautomer thereof, wherein Ring A is a group of formula:

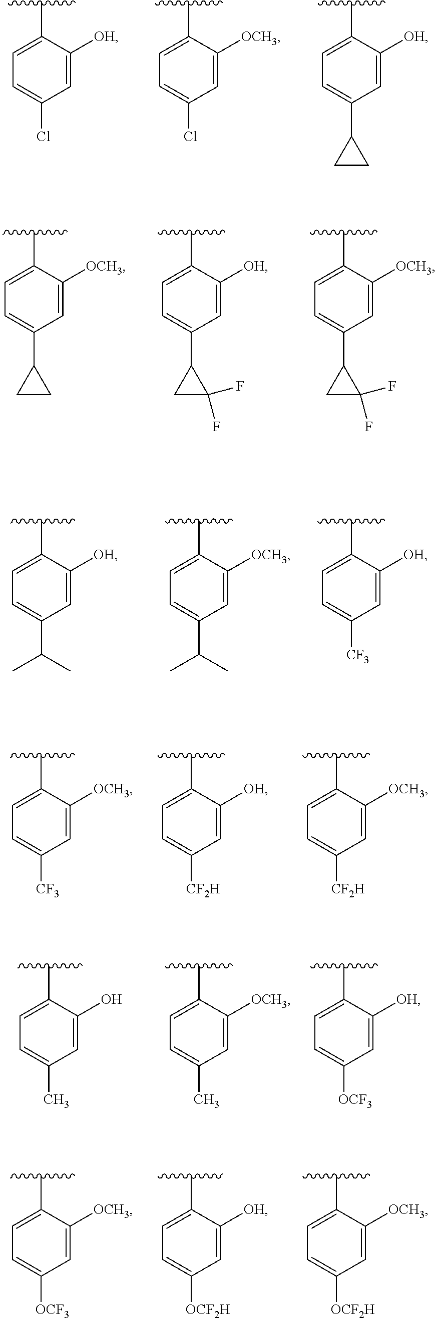

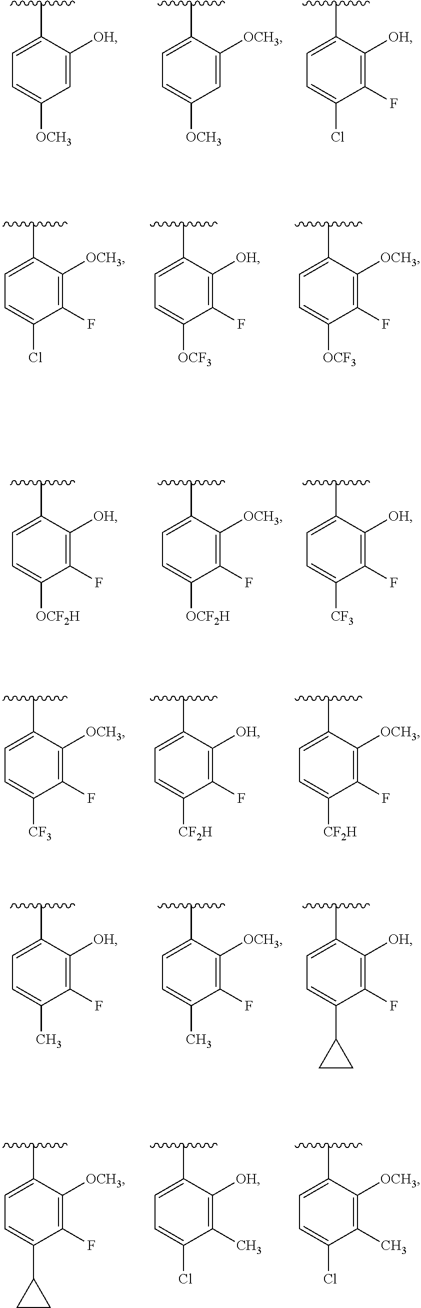

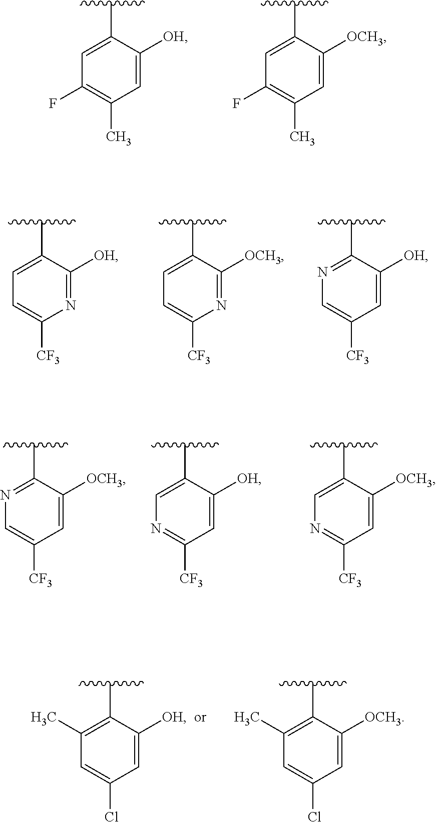

31. The compound of any one of the preceding claims, or a pharmaceutically acceptable salt or tautomer thereof, wherein Ring B of formula:

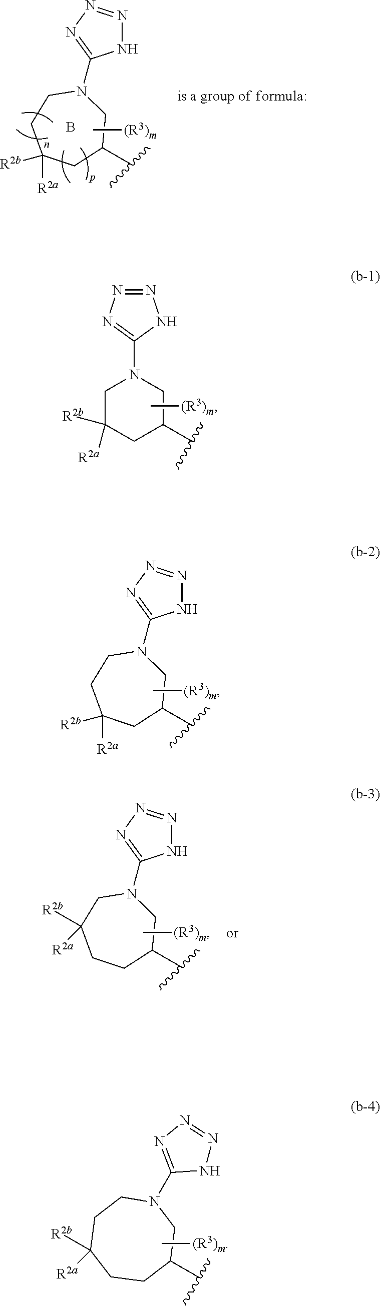

32. The compound of claim 31, or a pharmaceutically acceptable salt or tautomer thereof, wherein Ring B is a group of formula: 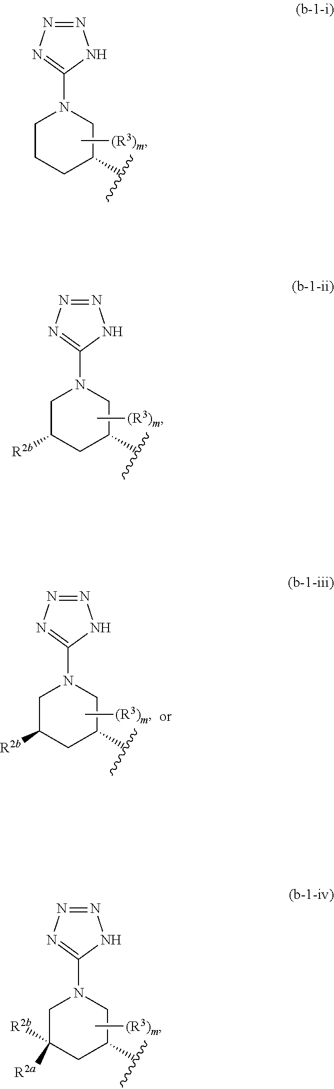
wherein each instance of R^{2a} and R^{2b} is independently halo, C₁₋₆ alkyl, C₁₋₆haloalkyl, C₃-C₄ carbocyclyl, or 3-4 membered heterocyclyl.

33. The compound of claim 31, or a pharmaceutically acceptable salt or tautomer thereof, wherein Ring B of formula (b-1), (b-2), (b-3), or (b-4), when two R³ groups are joined to form a C₁₋₃ alkylene bridging group or C₁₋₃ haloalkylene bridging group, is a group of formula: 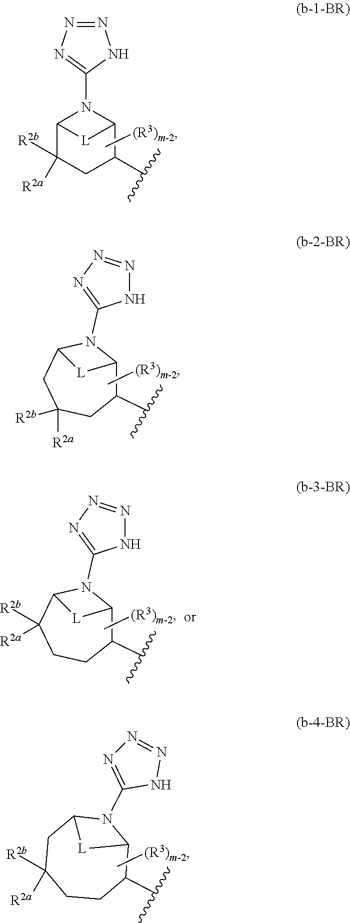
wherein L is a C₁₋₃ alkylene bridging group or C₁₋₃ haloalkylene bridging group.

34. The compound of claim 33, or a pharmaceutically acceptable salt or tautomer thereof, wherein Ring B is a group of formula: 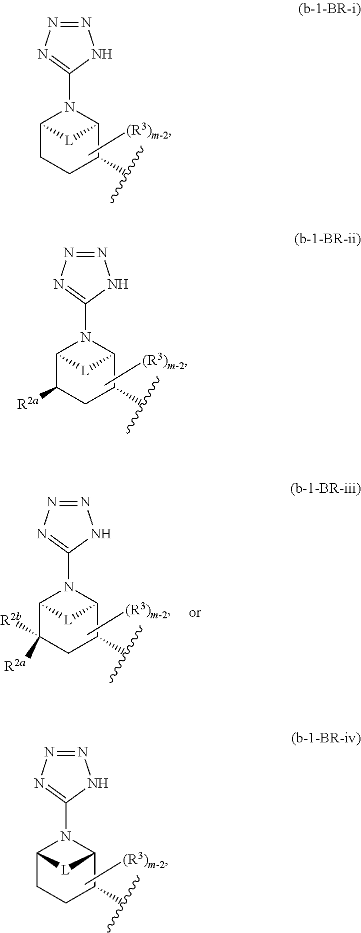
wherein each instance of R^{2a} and R^{2b} is independently halo, C₁₋₆ alkyl, C₁₋₆haloalkyl, C₃-C₄ carbocyclyl, or 3-4 membered heterocyclyl.

35. The compound of any one of claims 1-32, or a pharmaceutically acceptable salt or tautomer thereof, wherein Ring B is a group of formula:

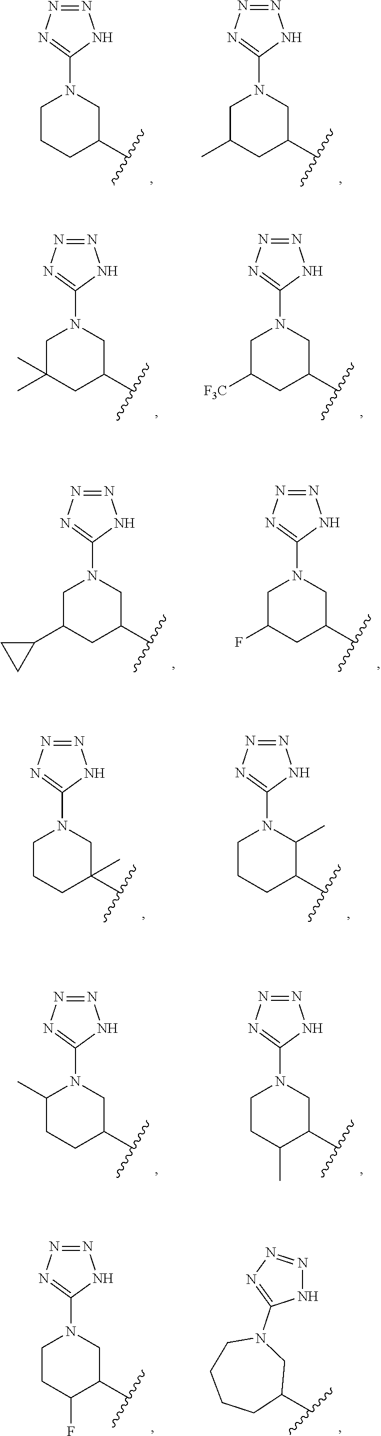

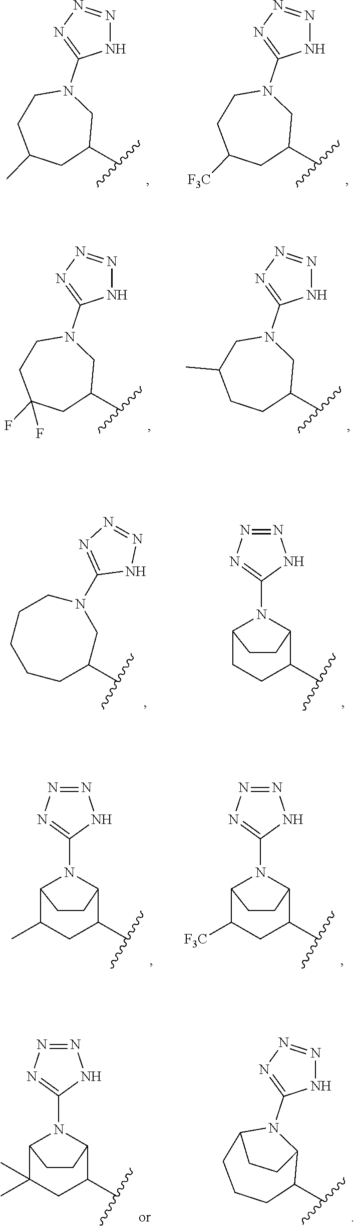

36. The compound of claim 35, or a pharmaceutically acceptable salt or tautomer thereof, wherein Ring B is a group of formula:

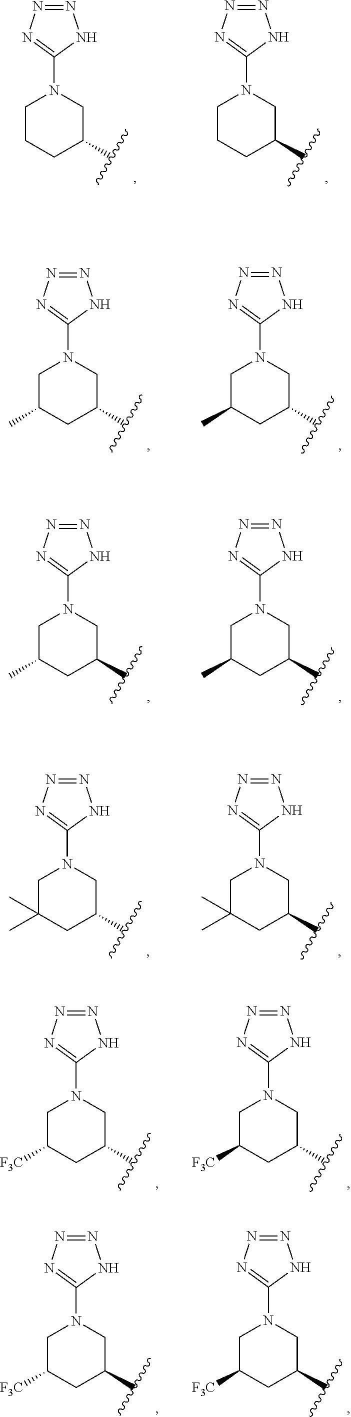

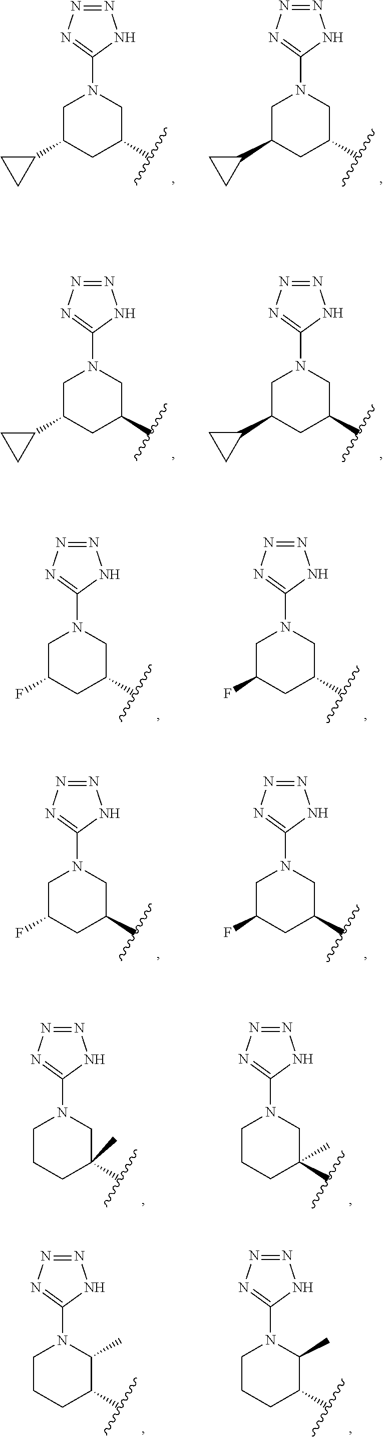

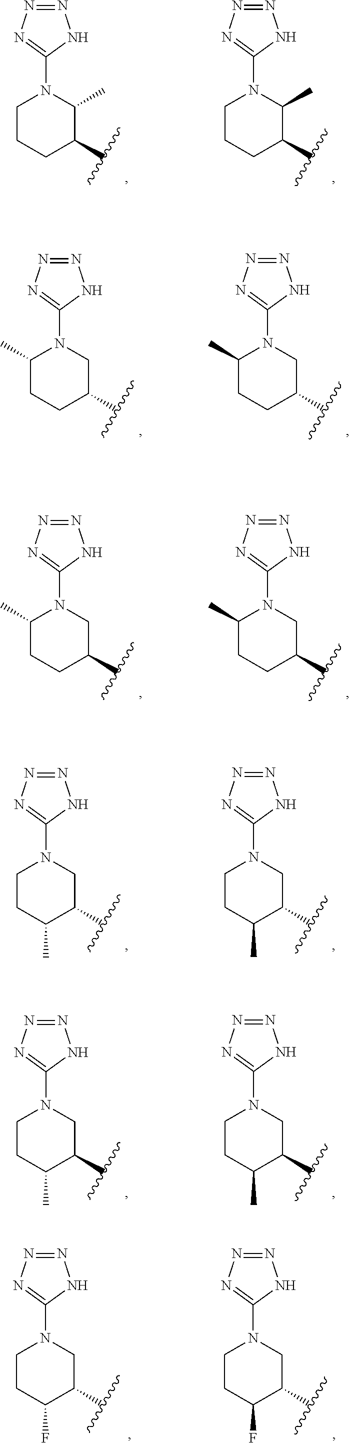

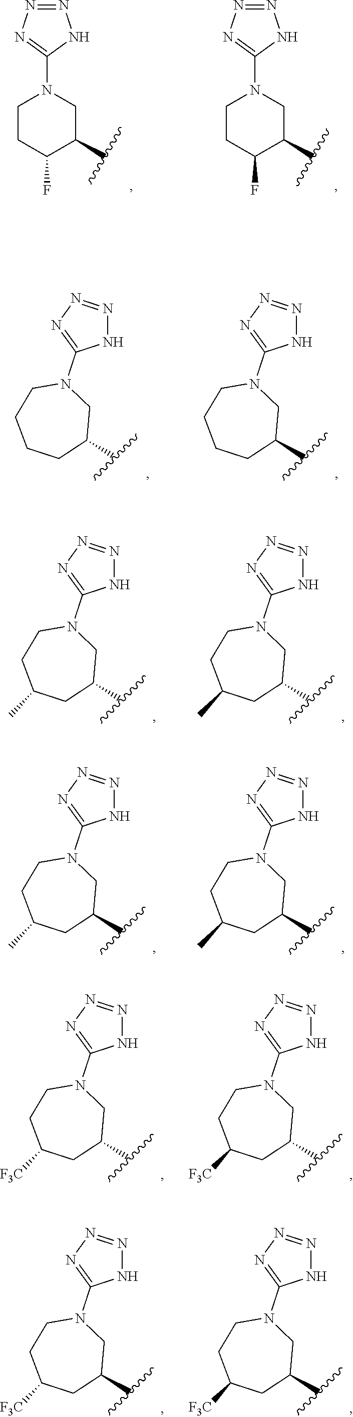

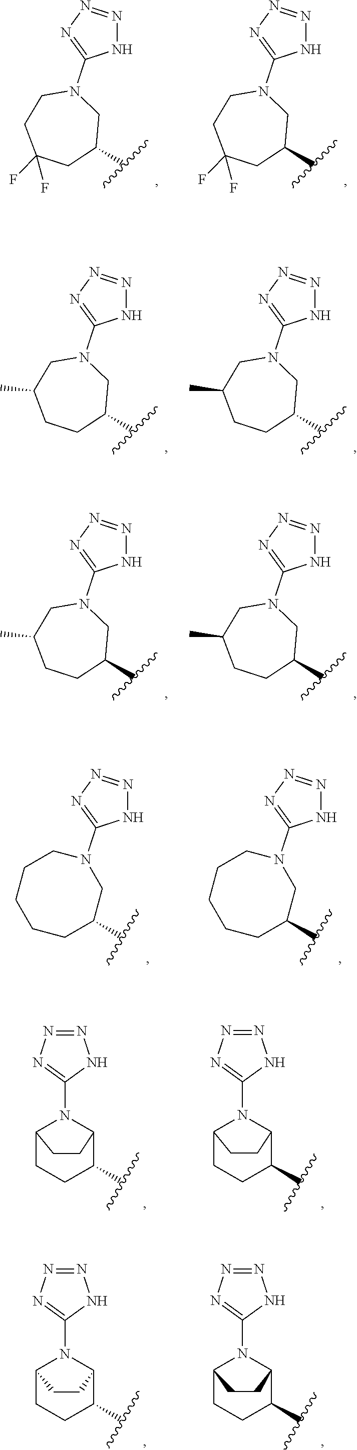

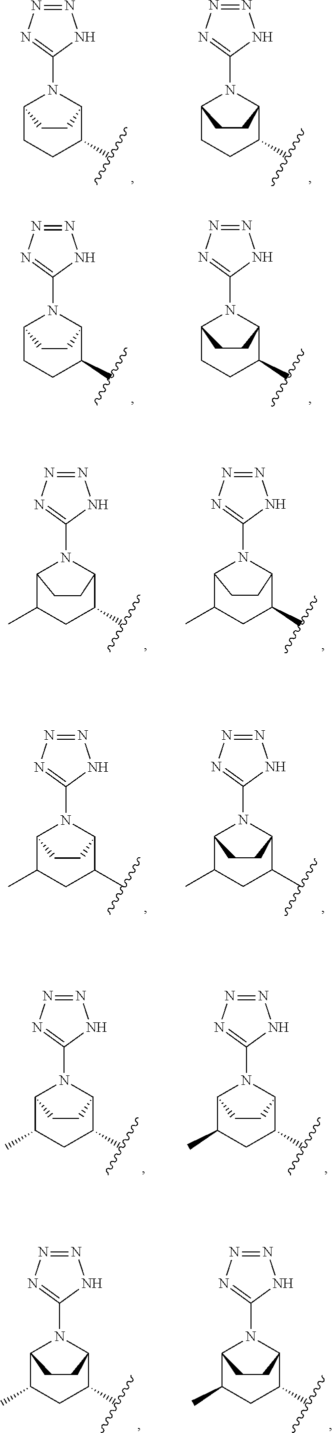

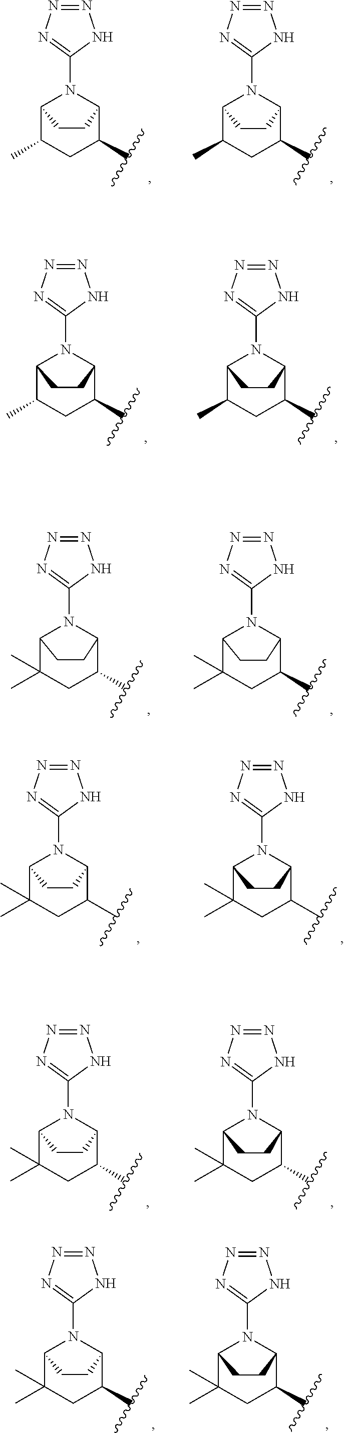

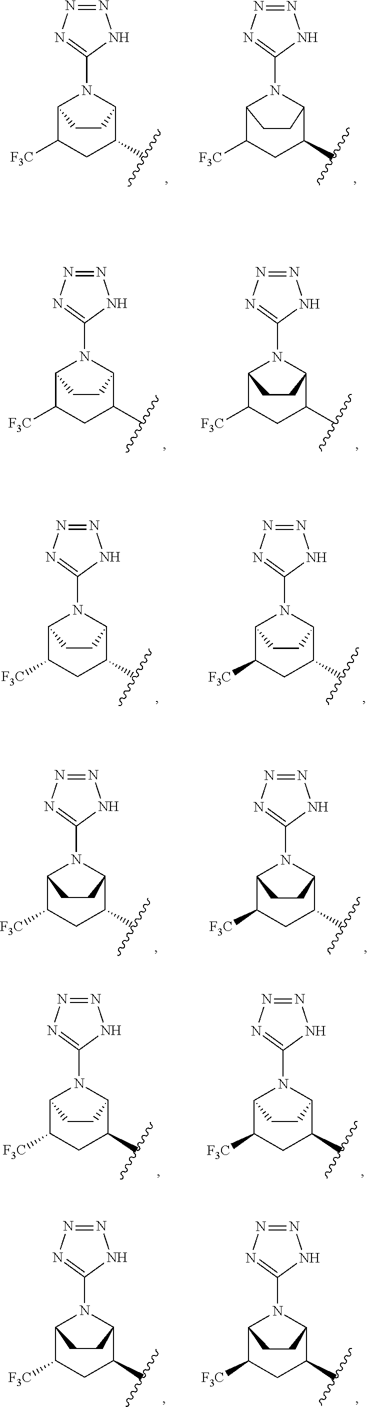

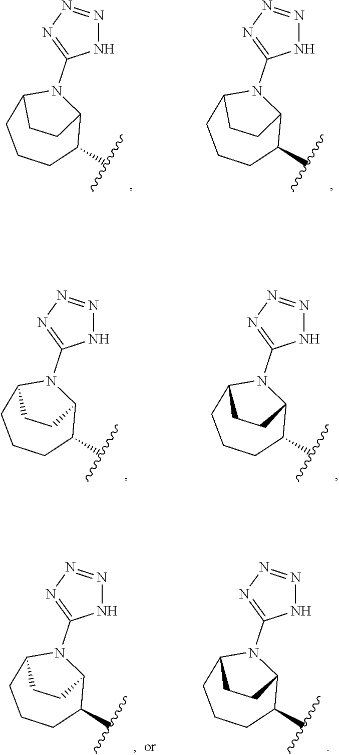 [SMILES: *[C@@H]1CCCC2CCC1N2c1nnn[nH]1 ; *[C@H]1CCCC2CCC1N2c1nnn[nH]1 ; *[C@@H]1CCC[C@@H]2CC[C@H]1N2c1nnn[nH]1 ; *[C@@H]1CCC[C@H]2CC[C@@H]1N2c1nnn[nH]1 ; *[C@H]1CCC[C@@H]2CC[C@H]1N2c1nnn[nH]1 ; *[C@H]1CCC[C@H]2CC[C@@H]1N2c1nnn[nH]1]

37. The compound of any one of the preceding claims, wherein the compound is selected from the compounds described in Table 1 or Table 2, or a pharmaceutically acceptable salt or tautomer thereof.

38. A pharmaceutical composition comprising the compound of any one of claims 1-37, or a pharmaceutically acceptable salt or tautomer thereof, and one or more pharmaceutically acceptable carriers.

39. A method of modulating NLRP3 activity, the method comprising administering to the subject a compound of any one of claims 1-37, or a pharmaceutically acceptable salt or tautomer thereof, or a pharmaceutical composition of claim 38.

40. A method of treating or preventing a disease or disorder, the method comprising administering to the subject a compound of any one of claims 1-37, or a pharmaceutically acceptable salt or tautomer thereof, or a pharmaceutical composition of claim 38.

41. The compound of any one of claims 1-37, or a pharmaceutically acceptable salt or tautomer thereof, or a pharmaceutical composition of claim 38, for use in treating or preventing a disease or disorder.

42. Use of the compound of any one of claims 1-37, or a pharmaceutically acceptable salt or tautomer thereof, in the manufacture of a medicament, for the treatment or prevention of a disease or disorder.

43. Use of the compound of any one of claims 1-37, or a pharmaceutically acceptable salt or tautomer thereof, for the treatment or prevention of a disease or disorder.

44. The method, compound, or use of any one of claims 39-43, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, obesity, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

45. A process for preparing a compound of Formula (I-B) of any one of the preceding claims, or a salt or tautomer thereof, wherein the compound is synthesized according to General Schemes A or B.
